(19) United States
(12) Patent Application Publication
(10) Pub. No.: US 2024/0033377 A1
(43) Pub. Date: Feb. 1, 2024

(54) AAV VECTORS FOR GENE EDITING

(71) Applicant: Scribe Therapeutics Inc., Alameda, CA (US)

(72) Inventors: Manuel MOHR, Berkeley, CA (US); Katherine BANEY, Berkeley, CA (US); Angus SIDORE, Oakland, CA (US); Cécile FORTUNY, Oakland, CA (US); Maroof ADIL, Davis, CA (US); Addison WRIGHT, El Cerrito, CA (US); Brett T. STAAHL, Tiburon, CA (US); Sean HIGGINS, Alameda, CA (US); Benjamin OAKES, El Cerrito, CA (US); Suraj MAKHIJA, San Francisco, CA (US); Sarah DENNY, San Francisco, CA (US)

(21) Appl. No.: 18/266,076

(22) PCT Filed: Dec. 9, 2021
(86) PCT No.: PCT/US2021/062714

§ 371 Date: Jun. 8, 2023

Related U.S. Application Data
(60) Provisional application No. 63/235,638, filed on Aug. 20, 2021.
(60) Provisional application No. 63/123,112, filed on Dec. 9, 2020.

(51) Int. Cl. A61K 48/00; C12N 9/22; C12N 15/86; C12N 15/11

(52) U.S. Cl. CPC A61K 48/005; C12N 9/22; C12N 15/86; C12N 15/11; C12N 2310/20; C12N 2750/14143; C12N 2750/14151

(57) ABSTRACT

Provided herein polynucleotides configured for incorporation into recombinant adeno-associated virus (AAV) vectors. The polynucleotides encode for CRISPR proteins, gRNA, and ancillary components of AAV vectors useful in the modification of target nucleic acids. The systems are also useful for introduction into cells, for example eukaryotic cells having mutations in the target nucleic acid of a gene. Also provided are methods of using such AAV vectors to modify cells having such mutations.

### CROSS-REFERENCE TO RELATED APPLICATIONS

This application claims priority to U.S. provisional patent application Nos. 63/123,112, filed on Dec. 9, 2020, and 63/235,638, filed on Aug. 20, 2021, the contents of which are incorporated by reference in their entirety herein.

### INCORPORATION BY REFERENCE OF SEQUENCE LISTING

The sequence listing paragraph application contains a Sequence Listing which has been submitted in ASCII format via EFS-WEB and is hereby incorporated by reference in its entirety. Said ASCII copy, created on Dec. 9, 2021 is named SCRB-028_02WO_SeqList_ST25.txt and is 13 MB in size.

### BACKGROUND

Gene editing holds great promise for treating or preventing many genetic diseases. However, safe and targeted delivery of CRISPR gene editing machinery into the desired cells is necessary to achieve therapeutic benefit. There remains a need in the art for compositions and methods for delivering CRISPR gene editing machinery to cells in vitro and/or in vivo.

### SUMMARY

The present disclosure relates to AAV vectors for the delivery of CRISPR nucleases to cells for the modification of target nucleic acids.

In some embodiments, the present disclosure provides polynucleotides useful for production of AAV transgenes (transgene plasmids for example), as well as for the production of recombinant adeno-associated virus (AAV) vectors. In some embodiments, the disclosure provides polynucleotides encoding a first adeno-associated virus (AAV) 5′ inverted terminal repeat (ITR) sequence, a second AAV 3′ ITR sequence, a CRISPR nuclease, a first guide RNA (gRNA), one or more promoters and, optionally, accessory elements; all encompassed in a single expression cassette capable of being incorporated into a single AAV particle. In other embodiments, the polynucleotides comprise sequences encoding a first 5′ AAV ITR sequence, a second 3′ AAV ITR sequence, a CRISPR nuclease, a first gRNA, a first promoter, a second promoter, and, optionally, one or more accessory elements. In still other embodiments, the polynucleotides comprise sequences encoding a first 5′ AAV ITR sequence, a second 3′ AAV ITR sequence, a CRISPR nuclease, a first gRNA, a second gRNA, a first promoter, a second promoter, a third promoter, and, optionally, one or more accessory elements.

In some embodiments, the sequence encoding the CRISPR protein and the gRNA sequence is less than about 3100, less than about 3090, less than about 3080, less than about 3070, less than about 3060, less than about 3050, or less than about 3040 nucleotides in combined length. In other embodiments, the polynucleotide encoding the CRISPR protein sequence and the gRNA sequence are less than about 3040 to about 3100 nucleotides in combined length.

In some embodiments, the polynucleotide sequences of the first promoter and the at least one accessory element are greater than at least about 1300, at least about 1350, at least about 1360, at least about 1370, at least about 1380, at least about 1390, at least about 1400, at least about 1500, at least about 1600 nucleotides, at least 1650, at least about 1700, at least about 1750, at least about 1800, at least about 1850, or at least about 1900 nucleotides in combined length. In other embodiments, the polynucleotide sequences of the first promoter, the second promoter, and two or more accessory elements are greater than at least about 1300 to at least about 1900 nucleotides in combined length. In some embodiments, the polynucleotide sequences of the first promoter, the second promoter, and the two or more accessory elements are greater than 1314 nucleotides in combined length. In other embodiments, the polynucleotide sequences of the first promoter, the second promoter, and the two or more accessory elements are greater than 1381 nucleotides in combined length. In one embodiment, the polynucleotide sequences of the first promoter, the second promoter, and the two or more accessory elements comprise at least 25%, 26%, 27%, 28%, 29%, 30%, 31%, 32%, 33%, 34%, or at least 35% or more of the total polynucleotide sequence length.

In some embodiments, the accessory element of the polynucleotide is selected from the group consisting of a poly(A) signal, a gene enhancer element, an intron, a posttranscriptional regulatory element, a nuclear localization signal (NLS), a deaminase, a DNA glycosylase inhibitor, a stimulator of CRISPR-mediated homology-directed repair, and an activator or repressor of transcription. In some embodiments, the accessory elements enhance the expression, binding, activity, or performance of the CRISPR protein as compared to the CRISPR protein in the absence of said accessory element. In particular embodiments, the enhanced performance is an increase in editing of a target nucleic acid upon expression of the CRISPR components in an in vitro assay of at least about 10%, at least about 20%, at least about 30%, at least about 40%, at least about 50%, at least about 60%, at least about 70%, at least about 80%, at least about 90%, at least about 100%, at least about 1500%, at least about 200%, or at least about 300%.

In some embodiments, the present disclosure provides a polynucleotide encoding a CRISPR protein that is a Class 2, Type V CRISPR protein. In some embodiments, the Class 2, Type V CRISPR protein is a CasX. In some embodiments, the CasX comprises a sequence selected from the group consisting of SEQ ID NOS: 1-3 and the sequences of SEQ ID NOS: 49-160, 40208-40369 and 40828-40912, or a sequence having at least 85%, at least 90%, at least 95%, at least 95%, at least 96%, at least 97%, at least 98%, or at least 99% identity thereto. In some embodiments, the present disclosure provides a polynucleotide encoding a Class 2, Type V CRISPR protein wherein the encoded CRISPR protein comprises the sequence of SEQ ID NO: 145 comprising at least one modification in one or more domains, wherein the one or more modifications are selected from the group consisting of the modifications set forth in Tables 30-33, wherein the one or more modifications results in an improved characteristic relative to the CRISPR protein of SEQ ID NO: 145.

In some embodiments, the polynucleotide encodes a first and a second gRNA wherein the encoded gRNA each comprise a sequence selected from the group of sequences of SEQ ID NOS: 2101-2285, 39981-40026, 40913-40958, and 41817 as set forth in Table 2, or a sequence having at least 85%, at least 90%, at least 95%, at least 95%, at least 96%, at least 97%, at least 98% identity thereto. In some embodiments, the encoded first and second gRNA comprise a scaffold sequence having one or more modifications relative to SEQ ID NO: 2238, wherein the one or more modifications result in an improved characteristic in the expressed first and second gRNA, wherein the one or more modifications comprise one or more nucleotide substitutions, insertions, and/or deletions as set forth in Table 28, wherein the one or more modifications result in an improved characteristic in the expressed first and second gRNA. In another embodiment, the encoded first and second gRNA comprise a scaffold sequence having one or more modifications relative to SEQ ID NO: 2239, wherein the one or more modifications result in an improved characteristic in the expressed first and second gRNA, wherein the one or more modifications comprise one or more nucleotide substitutions, insertions, and/or deletions as set forth in Table 28, wherein the one or more modifications result in an improved characteristic in the expressed first and second gRNA.

In some embodiments, the polynucleotide comprises 5′ and 3′ ITRs, wherein the ITRs are derived from serotype AAV1, AAV2, AAV3, AAV4, AAV5, AAV6, AAV7, AAV8, AAV9, AAV10, AAV11, AAV12, AAV 44.9, AAV-Rh74, or AAVRh10.

In some embodiments, the polynucleotide comprises one or more sequences selected from the group consisting of the sequences of Tables 8-10, 12, 13, and 17-22 and 24-27, or a sequence having at least 85%, at least 90%, at least 95%, at least 95%, at least 96%, at least 97%, at least 98%, or at least 99% identity thereto.

In other embodiments, the present disclosure provides a recombinant adeno-associated virus (rAAV) comprising an AAV capsid protein, and the polynucleotide of any one of the embodiments disclosed herein. In some embodiments, the AAV capsid protein is derived from serotype AAV1, AAV2, AAV3, AAV4, AAV5, AAV6, AAV7, AAV8, AAV9, AAV10, AAV11, AAV12, AAV 44.9, AAV-Rh74, or AAVRh10.

In some embodiments, the present disclosure provides a method of making a recombinant AAV vector, comprising providing a population of cells, and transfecting the population of cells with a vector comprising the polynucleotide of any of the embodiments disclosed herein. In some embodiments, the population of cells expresses the AAV rep and cap proteins.

In some embodiments, the present disclosure provides AAV vectors wherein one or more component sequences are selected from the group consisting of 5′ ITR, 3′ ITR, pol III promoter, pol II promoter, encoding sequence for CRISPR nuclease, encoding sequence for gRNA, accessory element, and poly(A) are substantially depleted of CpG dinucleotides, wherein the component sequences retain their functional characteristics (e.g., the ability to drive expression or the ability to retain editing potential for a target nucleic acid). In some embodiments, the AAV vectors that are substantially depleted of CpG dinucleotides exhibit reduced immunogenic properties (e.g., reduced ability to elicit inflammatory cytokines or antibodies to components of the AAV), e.g. when administered.

In some embodiments, the present disclosure provides a method for modifying a target nucleic acid in a population of mammalian cells, comprising contacting a plurality of the cells with an effective amount of the rAAV of any of the embodiments disclosed herein, wherein the target nucleic acid of a gene of the cells targeted by the expressed gRNA is modified by the expressed CRISPR protein.

In some embodiments, the present disclosure provides a method for treating a disease in a subject (e.g. a human) caused by one or more mutations in a gene of the subject, comprising administering a therapeutically effective dose of the rAAV of any of the embodiments disclosed herein.

In some embodiments, the present disclosure provides a method of reducing the immunogenicity of an rAAV, comprising deleting all or a portion of the CpG dinucleotides of the sequences of the AAV components selected from the group consisting of 5′ ITR, 3′ ITR, pol III promoter, pol II promoter, encoding sequence for CRISPR nuclease, encoding sequence for gRNA, accessory element, and poly(A).

### INCORPORATION BY REFERENCE

All publications, patents, and patent applications mentioned in this specification are herein incorporated by reference to the same extent as if each individual publication, patent, or patent application was specifically and individually indicated to be incorporated by reference.

The contents of WO/2020/247882, published Dec. 10, 2020 and PCT/US2021/061673, filed Dec. 2, 2021, are incorporated by reference in their entireties herein.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 shows a schematic of the AAV construct described in Example 1.

FIG. 2 shows results of an editing assay using AAV transgene plasmids nucleofected into mNPCs, as described in Example 1, demonstrating that the CasX and targeting guide in three different vectors (constructs 1, 2, and 3) edits on target (tdTomato) with high efficiency compared to non-targeting control (NT). Editing was assessed by FACS 5 days post-transfection. Data are presented as mean±SEM for n=3 replicates.

FIG. 3 shows results of an editing assay using AAV transgene plasmids nucleofected into mNPCs at four different dose levels, as described in Example 1. CasX delivered as an AAV transgene plasmid to mNPCs edits on target with high efficiency in a dose-dependent manner, compared to non-targeting control (NT). CasX variant 491 with scaffold variant 174 and spacer targeting tdTomato in three different vectors (constructs 1, 2, and 3) were nucleofected in mNPCs, and editing was assessed by FACS 5 days post-transfection. Data are presented as mean±SEM for n=3 replicates.

FIG. 4 shows results of an editing assay using AAV vector construct 3 transduced into mNPCs at 3-fold dilutions, assessed by FACS five days post-transduction, as described in Example 1. Data are presented as mean±SEM for n=3 replicates. MOI: multiplicity of infection.

FIG. 5 is a scanning transmission micrograph showing AAV particles with packaged CasX variant 438, gRNA scaffold 174 and spacer 12.7, as described in Example 2. AAV were negatively stained with 1% uranyl acetate. Empty particles are identified by a dark electron dense circle at the center of the capsid.

FIG. 6 shows results of an immunohistochemistry staining of mouse coronal brain sections, as described in Example 3. Mice received an ICV injection of 1×10¹¹ AAV packaged with CasX 491, gRNA scaffold 174 with spacer 12.7 (top panel), which were able to edit the tdTom locus in the Ai9 mice (edited cells appear white). The bottom panel shows that CasX 491 and scaffold 174 with a non-targeting spacer administered as an AAV ICV injection did not edit at the tdTom locus. Tissues were processed for immunohistochemical analysis 1 month post-injection.

FIG. 7 shows the results of an editing assay of the tdTom locus in mNPCs using AAV transgene plasmids of constructs having variations in the CasX promoters, as described in Example 4. Editing was assessed by FACS 5 days post-transfection. Data are presented as mean±SEM for n=3 replicates.

FIG. 8 shows the results of an editing assay of the tdTom locus in mNPCs using AAV transgene plasmids of constructs having variations in the CasX promoters, as described in Example 4. Editing was assessed by FACS 5 days post-transfection. Data are presented as mean±SEM for n=3 replicates.

FIG. 9 shows the results of an editing assay of the tdTom locus in mNPCs using AAV transgene plasmids of constructs having variations in the CasX promoters and transgene size (see table insert), as described in Example 4. Editing was assessed by FACS 5 days post-transfection. Data are presented as mean±SEM for n=3 replicates.

FIG. 10 shows the results of an editing assay of the tdTom locus in mNPCs using AAV vectors incorporating the same promoters as shown in FIG. 9, as described in Example 4. The graph on the left are results testing 3-fold dilutions of the constructs, while the graph on the right are results of editing using an MOI of 2×10⁵ vg/cell. Editing was assessed by FACS 5 days post-transfection. Data are presented as mean±SEM for n=3 replicates.

FIG. 11 shows the results of an editing assay of the tdTom locus in mNPCs using AAV vectors with protein promoter variants designed to reduce transgene size, compared to AAV with the top 4 protein promoter variants identified previously (AAV.3, AAV.4, AAV.5 and AAV.6), as described in Example 4. Editing was assessed by FACS 5 days post-transfection. Data are presented as mean±SEM for n=3 replicates. The dashed line shows editing levels of AAV.4, the AAV construct that in this experiment was used as a baseline for comparison across the variants.

FIG. 12 is a graph of percent editing versus transgene size for all constructs having varying promoters tested in this study. Constructs circled with dashes were identified as having above average editing while minimizing transgene size. The dashed line shows editing levels of AAV.4, the AAV construct that in this experiment was used as a baseline for comparison across variants.

FIG. 13 shows the results of an editing assay of mNPCs using AAV transgene plasmids having variations in gRNA promoter strength, as described in Example 5. Editing was assessed by FACS 5 days post-transfection. Data are presented as mean±SEM for n=3 replicates.

FIG. 14 shows the results of an editing assay of mNPCs using three different AAV vectors having variations in gRNA promoter strength, as described in Example 5. The graph on the left are results testing 3-fold dilutions of the constructs ranging from 1×10⁴ to 5×10⁵ vg/cell, while the graph on the right are results of editing using an MOI of 3×10⁵ vg/cell. Editing was assessed by FACS 5 days post-transfection. Data are presented as mean±SEM for n=3 replicates.

FIG. 15 is a bar graph that shows percent editing of the tdTom locus in mNPCs in an experiment to assess use of truncated U6 RNA promoters in constructs when delivered in AAV transgene plasmids designed to minimize the footprint of the Pol III promoter in the delivered transgene, as described in Example 5. Editing was assessed by FACS 5 days post-transfection. Data are presented as mean±SEM for n=3 replicates.

FIG. 16 is a bar graph that shows percent editing of the tdTom locus in mNPCs comparing base construct 53 to construct 85, when delivered as AAV vector designed to minimize the footprint of the Pol III promoter in the delivered transgene, as described in Example 5.

FIG. 17 is a bar graph that shows editing results of the tdTom locus in an experiment to assess the effects of constructs having engineered U6 RNA promoters when delivered to mNPCs in an AAV vector designed to minimize the footprint of the Pol III promoter in the AAV transgene, as described in Example 5. Editing was assessed by FACS 5 days post-transfection. Data are presented as mean±SEM for n=3 replicates.

FIG. 18 is a scatter plot depicting transgene size of all AAV variants tested having engineered U6 RNA promoters on the X-axis vs. percent of mNPCs edited on the Y-axis, as described in Example 5. The dashed line indicates construct 53, having the largest promoter tested, while the dotted line indicates construct 89, having the smallest promoter tested.

FIG. 19 shows the results of an editing assay of the tdTom locus in mNPCs in an experiment to assess the effects of constructs having engineered Pol III RNA promoters when delivered in an AAV vector designed to minimize the footprint of the Pol III promoter in the AAV transgene, as described in Example 5. Editing was assessed by FACS 5 days post-transfection. Data are presented as mean±SEM for n=3 replicates.

FIG. 20 is a bar graph showing AAV-mediated editing level in mNPCs at an MOI of 3.0E+5 vg/cell using the indicated constructs, as described in Example 5.

FIG. 21 is a scatter plot depicting the transgene size of all variants tested on the X-axis vs. the percent of mNPCs edited on the Y-axis, as described in Example 5.

FIG. 22 shows the results of an editing assay of the tdTom locus in mNPCs using AAV transgene plasmids having variations in poly(A) signals, as described in Example 6. Data are presented as mean±SEM for n=3 replicates.

FIG. 23 shows the results of an editing assay of the tdTom locus in mNPCs using two AAV vectors having the top poly(A) signals, as described in Example 6. Editing was assessed by FACS 5 days post-transfection. Data are presented as mean±SEM for n=3 replicates.

FIG. 24 are schematics of AAV plasmid constructs containing guide RNA transcriptional units (gRNA scaffold-spacer stack driven by a U6 promoter) in different orientations in regards to the protein promoter transcriptional unit, as described in Example 7. The tapered points depicts the orientation of the transcriptional unit for protein or guide RNA.

FIG. 25 shows the results of an editing assay of the tdTom locus in mNPCs using AAV transgene plasmids having differences in regulatory element orientation, as described in Example 7. Editing was assessed by FACS 5 days post-transfection. Data are presented as mean±SEM for n=3 replicates.

FIG. 26 shows the results of an editing assay of NPCs using AAV vectors containing guide RNA transcriptional units (gRNA scaffold-spacer stack driven by a U6 promoter) in different orientations in relation to the protein promoter transcriptional unit, as described in Example 7. The graph on the left shows results testing 3-fold dilutions of the constructs ranging from 1×10⁴ to 2×10⁶ vg/cell. The bar graph on the right shows AAV-mediated percent editing in mNPCs at an MOI of 3.0E+5 vg/cell. Editing was assessed by FACS 5 days post-transfection. Data are presented as mean±SEM for n=3 replicates.

FIG. 27 is a bar graph of results of an editing assay of the tdTom locus in mNPCs using AAV transgene plasmid constructs having different post-transcriptional regulatory elements compared to constructs not having post-transcriptional regulatory elements, as described in Example 8. Editing was assessed by FACS 5 days post-transfection. Data are presented as mean±SEM for n=3 replicates.

FIG. 28 is bar graph showing AAV-mediated editing levels (grey bars) of mNPCs at a viral MOI of 3.0E+5 compared to nucleofection editing using 150 ng of AAV-cis plasmids (dark bars) expressing the CasX protein 491 under the control of top promoters without (constructs 4, 5, 6) or in combination with different post-transcriptional regulatory element sequences (constructs 35-37 for base plasmid 4, constructs 38-39 for base plasmid 5, and constructs 42-43 for base plasmid 6), as described in Example 8. Editing was assessed by FACS 5 days post-transfection. Data are presented as mean±SEM for n=3 replicates.

FIG. 29 is a bar graph showing AAV-mediated editing levels of mNPCs at a viral MOI of 3.0E+5 for constructs under promoters without (constructs 58, 59, 53) or in combination of different post-transcriptional regulatory element sequences (respectively constructs 72-74 for base plasmid 58 containing Jet promoter, constructs 75-77 for base plasmid 59 containing Jet+USP promoter, and constructs 80-81 for base plasmid 53 containing UbC promoter), as described in Example 8. Editing was assessed by FACS 5 days post-transfection. Data (n=3) are presented as mean±SEM.

FIG. 30 is a scatterplot comparing the transgene size of each construct evaluated (from ITR to ITR, in bp) to AAV-mediated editing levels in mNPCs at a MOI of 3.0e+5 vg/cell, as described in Example 8. The circled data points represent the top identified constructs in terms of editing levels of select transgene size. The horizontal grey line shows the editing level of the benchmark vector AAV.53 for comparative purposes. The vertical grey line delimits vectors that are over or under a 4.9 kb transgene size.

FIG. 31 is a violin plot displaying AAV-mediated fold-improvement from the inclusion of the indicated PTRE element in the transgene plasmid, relative to its base (transgene with same promoter but no PTRE, indicated by gray dashed line), as described in Example 8.

FIG. 32 is a bar chart showing editing results of constructs with different neuronal enhancers delivered as AAV transgene plasmids to mNPCs, as described in Example 8. The gray lines show editing levels of reference plasmid 64, harboring CMV enhancer+core promoter. Editing was assessed by FACS 5 days post-transfection. Data are presented as mean±SEM for n=3 replicates.

FIG. 33 shows schematics of AAV constructs with alternative gRNA configurations for constructs having multiple gRNA, as described in Example 9. The top schematic is architecture 1, while the bottom is architecture 2. The tapered points depict the orientation of the transcriptional unit for protein or guide RNA.

FIG. 34 shows schematics of AAV constructs with alternative gRNA configurations for constructs having multiple gRNA, as described in Example 9. The tapered points depicts the orientation of the transcriptional unit for protein or guide RNA.

FIG. 35 shows schematics of guide RNA stack (Pol III promoter, scaffold, spacer) architectures tested with nucleofection and AAV transduction, as described in Example 9. Transgene harbors dual stacks in different orientations, with spacer 12.7, 12.2 and non-target spacer NT. The tapered points depict the orientation of the transcriptional unit for protein or guide RNA.

FIG. 36 shows the results of an editing assay for constructs having guide RNA stacks delivered via plasmid transfection to mNPCs, showing constructs with RNA stacks edit with enhanced potency compared to non-targeting control (NT), as described in Example 9. Editing was assessed by FACS 5 days post-transfection. Data are presented as mean±SEM for n=3 replicates.

FIG. 37 shows the results of an editing assay of mNPCs using AAV transgene plasmid constructs having multiple gRNA in different architectures and with different combinations of spacers (see FIG. 35) compared to construct 3 having a single gRNA and to a non-targeting construct, as described in Example 9. Editing was assessed by FACS 5 days post-transfection. Data are presented as mean±SEM for n=3 replicates.

FIG. 38 shows the results of an editing assay of mNPCs using AAV vector constructs 45-48 having multiple gRNA in different architectures and with different combinations of spacers (see FIG. 35) compared to construct 3, as described in Example 9. The left panel shows editing results using 3-fold MOI dilutions ranging from 1×10⁴ to 3×10⁵ vg/cell, while the right panel shows editing results at an MOI of 3×10⁵ vg/cell. Editing was assessed by FACS 5 days post-transfection. Data are presented as mean±SEM for n=3 replicates.

FIG. 39 is a bar graph of percent editing in mNPCs using AAV transgene plasmid constructs with varying 5′ NLS combinations (2, 7, and 9 in Table 15) with 3′ NLS 1, 8 and 9 in mNPCs, as described in Example 10.

FIG. 40 is a bar graph of percent editing in mNPCs using AAV vectors with varying 5′ NLS combinations with 3′ NLS 1, 8 and 9 in mNPCs, as described in Example 10.

FIG. 41 is a bar graph of percent editing in mNPCs using AAV vectors with varying NLS combinations when delivered in a vector designed to minimize the footprint of Pol III promoter in the transgene.

FIG. 42 is a schematic showing the organization of the components of an exemplary AAV transgene between the 5′ and 3′ ITRs, as described in Example 12.

FIG. 43A show results of editing assays in mNPCs nucleofected with 1000 of AAV-cis plasmids expressing CasX protein 491 expression of CMV and guide variants 174, 229-237 with spacer 11.30 targeting the mouse RHO exon 1 locus demonstrating improved activity at mouse RHO exon 1 in a dose-dependent manner, as described in Example 12. Triplicate wells were pooled together for gDNA extraction and therefore treated as n=1.

FIG. 43B is a bar graph showing fold-change in editing levels for each engineered scaffolds (229-237) relative to guide 174 with spacer 11.30 (set to a value of 1.0) across two plasmid nucleofection doses 1000 and 500 ng of AAV-cis plasmids, as described in Example 12. Triplicate wells were pooled together for gDNA extraction and therefore treated as n=1.

FIG. 44A show editing results of engineered guide 235 compared to 174 with spacer 11.1 targeting RHO at the exogenous RHO-GFP locus (with GFP as the reporter), under the expression of Pol III hU6 promote in ARPE-19 cells, demonstrating improved activity by the 235 variant at the human RHO locus, with increased on-target activity at WT exogenous RHO without off-target cleavage at the mutant RHO reporter gene, as described in Example 12. Data (n=3) are presented as mean±SD.

FIG. 44B is a bar graph displaying fold-change in editing levels of engineered guide 235 compared to 174 at the human RHO locus, with p590.4910.2350.11.1 normalized to benchmark p590.4910.1740.11.1 levels (set to value 1.0) in cells nucleofected with 1000 ng of each plasmid, as described in Example 12. Data (n=3) are presented as mean±SD.

FIG. 45A shows editing levels in mNPCs by AAV-mediated expression of CasX molecule and engineered guide variant 235 compared to guide scaffold 174 with spacer 11.30 at 3 different MOI levels, confirming increased editing levels at the endogenous mouse Rho exon 1 locus with no off-target locus, as described in Example 12.

FIG. 45B is a bar graph displaying fold-change in editing levels in mNPCs by AAV-mediated expression of CasX molecule and engineered guide variant 235 compared to guide scaffold 174 with spacer 11.30 in cells infected at a 5.0e+5 MOI, as described in Example 12. Data are presented as the mean of n=3.

FIG. 46A shows editing results at the human RHO locus in mNPCs nucleofected with 1000 and 500 ng of AAV-cis plasmids expressing CasX protein 491 and sgRNA-scaffold 174 with on-target spacers of varying length, demonstrating improved on-target editing at the mouse RHO locus, as described in Example 12. Spacers variants are: 11.30 (20 nt WT RHO), 11.38 (18 nt WT RHO), and 11.39 (19 nt WT RHO), respectively. A control spacer, no-target (NT), designed to not recognize any sequence across the mouse and human genomes, was also tested as a negative control to ensure no unspecific targeting resulting from the expression of the CasX protein alone. Triplicate wells were pooled together for gDNA extraction and therefore treated as n=1.

FIG. 46B is a bar graph showing editing levels at the human RHO locus in nucleofected mNPCs with 1000 ng of AAV-cis plasmids expressing CasX protein 491 and sgRNA-scaffold 174 with the indicated off-target spacers, as described in Example 12.

FIG. 46C is a bar graph displaying fold-change in editing levels at the human RHO locus in nucleofected mNPCs for each sgRNA-scaffold 174 with spacer variants 11.38 and 11.39 normalized to levels of parental sgRNA-scaffold-spacer 174.11.30, as described in Example 12. Data shows means+SD across 3 different biological replicates.

FIG. 47A is a Whisker box graph showing editing results of RHO in a mouse model comparing AAV-mediated delivery of sgRNA scaffold variants and optimized spacers compared to benchmark construct, as described in Example 13. Each dot represents one retina (n=8-16). One-way ANOVA statistical test was performed, ***=p<0.001.

FIG. 47B is a Whisker box graph showing the relative fold-change in editing of RHO in a mouse model comparing AAV-mediated delivery of sgRNA scaffold variants 174 and 235 and optimized spacers compared to benchmark construct, as described in Example 13. Values are relative to the benchmark vector AAV.RHO.174.11.30 (set to a value of 1). Each dot represents one retina (n=8-16).

FIG. 48A is a bar graph showing CTC-PAM editing levels (indel rates) at the mouse RHO locus in mNPCs nucleofected with 1000 and 500 ng of AAV-cis plasmids expressing the CasX protein variant 491, 515,527, 528, 535, 536 or 537, respectively, and sgRNA-scaffold 235.11.37 (on target), as described in Example 14. A control spacer, no-target (NT), designed to not recognize any sequence across the mouse and human genomes, was also tested as a negative control to ensure no unspecific targeting resulting from the expression of the CasX protein alone. Triplicate wells were pooled together for gDNA extraction and therefore treated as n=1.

FIG. 48B is a bar graph showing CTC-PAM editing levels (indel rates) at the mouse RHO locus in mNPCs nucleofected with AAV-cis plasmids expressing the CasX protein variant 491, 515, 527, 528, 535, 536 or 537, respectively, and sgRNA-scaffold 235.11.39 (off-target), as described in Example 14.

FIG. 48C shows a bar graph displaying fold-change in editing levels for each indicated CasX protein variant with guide 235 and spacer 11.39, with results normalized to levels of the parental CasX protein 491, as described in Example 14.

FIG. 49A shows a bar graph showing editing levels in ARPE-19 mNPC nucleofected with 1000 ng of AAV-cis plasmids expressing CasX protein variant 491, 515, 527, 528, 535, 536 or 537 and guide variant 235 with spacer 11.41 or 11.43, as described in Example 14. Data (n=3) are presented as mean±SD.

FIG. 49B shows a bar graph displaying fold-change in editing levels in ARPE-19 mNPC nucleofected with 1000 ng of AAV-cis plasmids expressing CasX protein variant 515, 527, 528, 535, 536 or 537 and guide variant 235 with spacer 11.41 or 11.43 relative to benchmark p59.491.235.11.41 levels (set to a value of 1.0), as described in Example 14. Data (n=3) are presented as mean±SD.

FIG. 50A shows a bar graph of AAV-mediated editing levels in mNPCs at the endogenous mouse Rho exon 1 locus, as described in Example 14. mNPCs were infected using a 3.0e+5 and 1.0e+5 vg/cell MOI with AAV vectors expressing the indicated CasX protein 491, 515, 527, 528, 535, or 537 and sgRNA-scaffold variant 235.11.39, as described in Example 14. Data (n=3) are presented as the mean.

FIG. 50B is a bar graph displaying fold-change in editing levels for the indicated CasX variant with guide scaffold 235 relative to guide 174 with spacer 11.39 in cells infected with the indicated MOI, as described in Example 14.

FIG. 51 is an illustration of reference mRHO exon 1 locus and target amino acid residue P23 (CCC) sequence (highlighted in bold), showing spacer 11.30 target sequence and expected CasX-mediated cleavage, as described in Example 15. The most common predicted edits quantified in CRISPResso edits (substitution/deletions) are displayed under the reference genome).

FIG. 52A shows results of in vivo AAV CasX-mediated editing of the mRHO P23 locus in retinae in C57BL6J mice (n=6-8; quantification in percent of total indels detected by NGS), as described in Example 15.

FIG. 52B shows the fraction (%) of AAV CasX-mediated frame-shift edits of the mRHO P23 locus in the retinae in C57BL6J (n=6-8) mice (n=6-8; quantification in percent of total indels detected by NGS), as described in Example 15.

FIGS. 53A-53F show representative fluorescence imaging of retinas from AAV-CasX treated mice or negative controls and stained, as described in Example 15. Cell nuclei were counterstained with DAPI (top row; FIGS. 53A-C) to visualized retinal layers and stained with HA-tag (bottom row, FIGS. 53D-F) antibody to detect CasX expression in photoreceptors (ONL) and other retinal layers (INL; GCL). Legends: ONL=Outer nuclear layer; INL=Inner nuclear layer, GCL=Ganglion cell layer.

FIG. 54A is a box plot showing median, minimal and highest editing values using AAV-mediated expression of CasX 491 detected by NGS 3 weeks post-injection in wild-type retinae injected with 5.0e+9 vg/eye of AAV.X.491.174.11.30 vectors, in which the 491 protein is driven by promoter variants designed to selectively express in rod photoreceptors (X=RP1-RP5) or a ubiquitous promoter (X=CMV), as described in Example 16. The grey line is placed at the editing levels achieved by AAV.RP1.491.174.11.30 to compare to other viral vectors tested.

FIG. 54B is a plot displaying levels of editing achieved by AAV vectors in wild-type retinae injected with 5.0e+9 vg/eye of AAV.X.491.174.11.30 vectors, compared to total transgene size (bp), as described in Example 16. The grey line delimitates transgenes below or above 4.9 kb size.

FIG. 55 shows in vivo editing results that AAV-mediated expression of CasX 491 and sgRNA spacer 174.4.76 in rod photoreceptors led to detectable levels of editing levels at integrated Nrl-GFP locus in a dose-dependent manner, as described in Example 16. The bar graph shows editing levels detected by NGS at the integrated GFP locus 4-weeks and 12-weeks post-injection in heterozygous Nrl-GFP mice injected with the indicated doses of AAV.RP1.491.174.4.76 vectors in one eye, and the vehicle control in the contralateral eye).

FIG. 56A shows a western blot of retinal lysates from positive (C1, uninjected homozygous Nrl-GFP retinae) and negative (N, uninjected C57BL/6J retinae) controls, vehicle groups (V, AAV formulation buffer injected retinae) and AAV-CasX 491, sgRNA 174 and spacer 4.76 treated retinae with the medium dose 1.9e+9 (M) or high dose 1.0e+10 vg (H arm. Blots display the respective bands for the HA protein (CasX protein, top), GFP protein (middle) and GAPDH (bottom panels) used as a loading control, as described in Example 16. Levels of percent editing in the retinae detected by NGS are displayed under the blot for each sample.

FIG. 56B is a scatter boxplot representing levels of GFP protein detected in the western blots of FIG. 56A (ratios of densitometric values of the GFP band for total amount of proteins, normalized to the vehicle group levels), as described in Example 16. One-way ANOVA statistical analysis was performed (*=p<0.5).

FIG. 56C is a plot correlating GFP protein fraction to levels of editing achieved in mouse retinae of the AAV-treated mice, for both the 1.0e+9 and 1.0e+10 dose groups, as described in Example 16.

FIG. 57A is a bar graph representing the ratio of GFP fluorescence levels (superior to inferior retina mean grey values) detected by fundus imaging at 4-weeks compared to 12-weeks post-injection in mice injected with two dose levels of AAV constructs, as described in Example 16.

FIG. 57B displays representative images of fluorescence fundus imaging of GFP in retina from mice injected with 1.0e+9 vg (#13) or 1.0e+10 vg (#34) with the AAV constructs at 4-weeks and (left panel) or 12-weeks (right panel), as described in Example 16.

FIGS. 58A-58L present histology images or retinae of mice stained with various immunochemistry reagents, as described in Example 16, confirming efficient knock-down of GFP in photoreceptor cells in an AAV-dose dependent manner. The images are representative confocal images of cross-sectioned retinae injected with vehicle (FIGS. 58A, 58B, 58C, 58D), AAV-CasX at a 1.0e+9 vg dose (FIGS. 58E, 58F, 58G, and 58H) and 1.0E+10 vg dose (FIGS. 58I, 58J, 58K, and 58L). Structural imaging shows GFP expression by rod photoreceptors in the outer segment (images in FIGS. 58A, 58E, 58I and images FIGS. 58C, 58G, and 58K for 20× and 40× magnifications, respectively). Cell nuclei were counterstained with Hoechst (FIGS. 58B, 58F, and 58J) and cells stained with anti-HA to correlate levels of HA (CasX transgene levels; FIGS. 58D, 58H, and 58L; 40× magnification) and GFP expressed in photoreceptors. White box outlines in B and F indicate retinal regions analyzed at 40× magnification in FIGS. 58C and 58G. Legend: RPE=retinal pigment epithelium, OS=outer segment, ONL=outer nuclear layer, INL=inner nuclear layer, GCL=ganglion.

FIG. 59A shows results of an immunohistochemistry staining of a mouse liver section showing that CasX 491 and scaffold 174 with spacer 12.7 administered as an AAV IV injection was able to edit the tdTom locus in vivo in Ai9 mice, as described in Example 3. The images are representative of n=3 animals.

FIG. 59B shows results of an immunohistochemistry staining of a mouse heart section showing that CasX 491 and scaffold 174 with spacer 12.7 administered as an AAV IV injection was able to edit the tdTom locus in vivo in Ai9 mice, as described in Example 3. The images are representative of n=3 animals.

FIG. 60 is a graph of the quantification of percent editing at the B2M locus 5 days post-transduction of AAVs into human NPCs in a series of three-fold dilution of MOI, as described in Example 17. Editing levels were determined by NGS as indel rate and by flow cytometry as population of cells that do not express the HLA protein due to successful editing at the B2M locus.

FIG. 61 shows the results of an editing assay measured as indel rate detected by NGS at the human AAVS1 locus in human induced neurons (iNs) using the three indicated AAVs, each containing CasX 491 and gRNA with a specific spacer targeting AAVS1, as described in Example 17.

FIG. 62 is a bar graph exhibiting percent editing at the B2M locus in human iNs 14 days post-transduction of AAVs expressing CasX 491 driven by various protein promoters at an MOI of 2E4 or 6.67E3, as described in Example 17.

FIG. 63 shows the results of an editing assay using AAV transgene plasmids nucleofected into hNPCs, as described in Example 18, demonstrating that CpG reduction or depletion within the U1a promoter (construct ID 178 and 179), U6 promoter (construct ID 180 and 181), or bGH poly(A) (construct ID 182) did not significantly reduce CasX-mediated editing at the B2M locus compared to the editing achieved with the original CpG⁺ AAV vector (construct ID 177). The controls used in this experiment were the non-targeting (NT) spacer and no treatment (NTx).

FIG. 64 is a bar graph showing editing results of the tdTomato locus in an experiment to assess the effects of AAV constructs having engineered Pol III promoter hybrid variants when delivered to mNPCs in an AAV vector, as described in Example 5. Editing was assessed by FACS five days post-nucleofection.

FIG. 65 is a schematic of the regions and domains of a guide RNA used to design a scaffold library, as described in Example 20.

FIG. 66 is a pie chart of the relative distribution and design of the scaffold library with both unbiased (double and single mutations) and targeted mutations (towards the triplex, scaffold stem bubble, pseudoknot, and extended stem and loop) indicated, as described in Example 20.

FIG. 67 is a schematic of the triplex mutagenesis designed to specifically incorporate alternate triplex-forming base pairs into the triplex, as described in Example 20. Solid lines indicate the Watson-Crick pair in the triplex; the third strand nucleotide is indicated as a dotted line representing the non-canonical interaction with the purine of the duplex. In the library, each of the 5 locations indicated was replaced with all possible triplex motifs (G:GC, T:AT, G:GC)=243 sequences. Sequence of ACUGGCGCUUUUAUCUGAUUACUUUGAGAGCCAUCANNNAUCAAAG (SEQ ID NO: 41829).

FIG. 68 is a bar chart with results of the enrichment values of reference guide scaffolds 174 and 175 in each screen, as described in Example 20.

FIG. 69 are scatterplots showing the log₂ enrichment value for each measured single nucleotide substitution, deletion, or insertion, as measured in each of two independent screens of the mutant libraries for guide scaffolds 174 and 175, as described in Example 20.

FIG. 70 are heat maps for single mutants in guide scaffolds 174 and 175 showing specific mutable regions in the scaffold across the sequences, as described in Example 20. Yellow shades reflect values with similar enrichment to the reference scaffolds; red shades indicate an increase in enrichment, and thus activity, relative to the reference scaffold; blue shades indicate a loss of activity relative to the wildtype scaffold; white indicates missing data (or a substitution that would result in wildtype sequence.

FIG. 71 is a scatterplot that compares the log₂ enrichment of single nucleotide mutations on reference guide scaffolds 174 and 175, as described in Example 20. Only those mutations to positions that were analogous between 174 and 175 are shown. Results suggest that, overall, guide scaffold 174 is more tolerant to changes than 175.

FIG. 72 is a bar chart showing the average (and 95% confidence interval) log 2 enrichment values for a set of scaffolds in which the pseudoknot pairs have been shuffled, such that each new pseudoknot has the same composition of base pairs, but in a different order within the stem, as described in Example 20. Each bar represents a set of scaffolds with the G:A (or A:G) pair location indicated (see diagram at right). 291 pseudoknot stems were tested; numbers above bars indicate the number of stems with the G:A (or A:G) pair at each position.

FIG. 73 is a schematic of the pseudoknot sequence of FIGS. 55 and 56, given 5′ to 3′, with the two strand sequences separated by an underscore.

FIG. 74 is a bar chart showing the average (and 95% confidence interval) log₂ enrichment values for scaffolds, divided by the predicted secondary structure stability of the pseudoknot stem region, as described in Example 20. Scaffolds with very stable stems (e.g., ΔG<−7 kcal/mol) had high enrichment values on average, whereas scaffolds with destabilized stems (ΔG≥−5 kcal/mol) had low enrichment values on average.

FIG. 75 is a heat map of all double mutants of positions 7 and 29 in scaffold 175, as described in Example 20. The pseudoknot sequence is given 5′ to 3′, on the right.

FIG. 76 is a graph of a survival assay to determine the selective stringency of the CcdB selection to different spacers when targeted by CasX protein 515 and Scaffold 174, as described in Example 21.

### DETAILED DESCRIPTION

While exemplary embodiments have been shown and described herein, it will be obvious to those skilled in the art that such embodiments are provided by way of example only. Numerous variations, changes, and substitutions will now occur to those skilled in the art without departing from the inventions claimed herein. It should be understood that various alternatives to the embodiments described herein may be employed in practicing the embodiments of the disclosure. It is intended that the claims define the scope of the invention and that methods and structures within the scope of these claims and their equivalents be covered thereby.

Unless otherwise defined, all technical and scientific terms used herein have the same meaning as commonly understood by one of ordinary skill in the art to which this invention belongs. Although methods and materials similar or equivalent to those described herein can be used in the practice or testing of the present embodiments, suitable methods and materials are described below. In case of conflict, the patent specification, including definitions, will control. In addition, the materials, methods, and examples are illustrative only and not intended to be limiting. Numerous variations, changes, and substitutions will now occur to those skilled in the art without departing from the invention.

### Definitions

The terms “polynucleotide” and “nucleic acid,” used interchangeably herein, refer to a polymeric form of nucleotides of any length, either ribonucleotides or deoxyribonucleotides. Thus, terms “polynucleotide” and “nucleic acid” encompass single-stranded DNA; double-stranded DNA; multi-stranded DNA; single-stranded RNA; double-stranded RNA; multi-stranded RNA; genomic DNA; cDNA; DNA-RNA hybrids; and a polymer comprising purine and pyrimidine bases or other natural, chemically or biochemically modified, non-natural, or derivatized nucleotide bases.

“Hybridizable” or “complementary” are used interchangeably to mean that a nucleic acid (e.g., RNA, DNA) comprises a sequence of nucleotides that enables it to non-covalently bind, i.e., form Watson-Crick base pairs and/or G/U base pairs, “anneal”, or “hybridize,” to another nucleic acid in a sequence-specific, antiparallel, manner (i.e., a nucleic acid specifically binds to a complementary nucleic acid) under the appropriate in vitro and/or in vivo conditions of temperature and solution ionic strength. It is understood that the sequence of a polynucleotide need not be 100% complementary to that of its target nucleic acid sequence to be specifically hybridizable; it can have at least about 70%, at least about 80%, or at least about 90%, or at least about 95% sequence identity and still hybridize to the target nucleic acid sequence. Moreover, a polynucleotide may hybridize over one or more segments such that intervening or adjacent segments are not involved in the hybridization event (e.g., a loop structure or hairpin structure, a ‘bulge’, ‘bubble’ and the like).

A “gene,” for the purposes of the present disclosure, includes a DNA region encoding a gene product (e.g., a protein, RNA), as well as all DNA regions which regulate the production of the gene product, whether or not such regulatory sequences are adjacent to coding and/or transcribed sequences. Accordingly, a gene may include accessory element sequences including, but not necessarily limited to, promoter sequences, terminators, translational regulatory sequences such as ribosome binding sites and internal ribosome entry sites, enhancers, silencers, insulators, boundary elements, replication origins, matrix attachment sites and locus control regions. Coding sequences encode a gene product upon transcription or transcription and translation; the coding sequences of the disclosure may comprise fragments and need not contain a full-length open reading frame. A gene can include both the strand that is transcribed as well as the complementary strand containing the anticodons.

The term “downstream” refers to a nucleotide sequence that is located 3′ to a reference nucleotide sequence. In certain embodiments, downstream nucleotide sequences relate to sequences that follow the starting point of transcription. For example, the translation initiation codon of a gene is located downstream of the start site of transcription.

The term “upstream” refers to a nucleotide sequence that is located 5′ to a reference nucleotide sequence. In certain embodiments, upstream nucleotide sequences relate to sequences that are located on the 5′ side of a coding region or starting point of transcription. For example, most promoters are located upstream of the start site of transcription.

The term “adjacent to” with respect to polynucleotide or amino acid sequences refers to sequences that are next to, or adjoining each other in a polynucleotide or polypeptide. The skilled artisan will appreciate that two sequences can be considered to be adjacent to each other and still encompass a limited amount of intervening sequence, e.g., 1, 2, 3, 4, 5, 6, 7, 8, 9 or 10 nucleotides or amino acids.

The term “accessory element” is used interchangeably herein with the term “accessory sequence,” and is intended to include, inter alia, polyadenylation signals (poly(A) signal), enhancer elements, introns, posttranscriptional regulatory elements (PTREs), nuclear localization signals (NLS), deaminases, DNA glycosylase inhibitors, additional promoters, factors that stimulate CRISPR-mediated homology-directed repair (e.g. in cis or in trans), activators or repressors of transcription, self-cleaving sequences, and fusion domains, for example a fusion domain fused to a CRISPR protein. It will be understood that the choice of the appropriate accessory element or elements will depend on the encoded component to be expressed (e.g., protein or RNA) or whether the nucleic acid comprises multiple components that require different polymerases or are not intended to be expressed as a fusion protein.

The term “promoter” refers to a DNA sequence that contains a transcription start site and additional sequences to facilitate polymerase binding and transcription. Exemplary eukaryotic promoters include elements such as a TATA box, and/or B recognition element (BRE) and assists or promotes the transcription and expression of an associated transcribable polynucleotide sequence and/or gene (or transgene). A promoter can be synthetically produced or can be derived from a known or naturally occurring promoter sequence or another promoter sequence. A promoter can be proximal or distal to the gene to be transcribed. A promoter can also include a chimeric promoter comprising a combination of two or more heterologous sequences to confer certain properties. A promoter of the present disclosure can include variants of promoter sequences that are similar in composition, but not identical to, other promoter sequence(s) known or provided herein. A promoter can be classified according to criteria relating to the pattern of expression of an associated coding or transcribable sequence or gene operably linked to the promoter, such as constitutive, developmental, tissue-specific, inducible, etc. A promoter can also be classified according to its strength. As used in the context of a promoter, “strength” refers to the rate of transcription of the gene controlled by the promoter. A “strong” promoter means the rate of transcription is high, while a “weak” promoter means the rate of transcription is relatively low.

A promoter of the disclosure can be a Polymerase II (Pol II) promoter. Polymerase II transcribes all protein coding and many non-coding genes. A representative Pol II promoter includes a core promoter, which is a sequence of about 100 base pairs surrounding the transcription start site, and serves as a binding platform for the Pol II polymerase and associated general transcription factors. The promoter may contain one or more core promoter elements such as the TATA box, BRE, Initiator (INR), motif ten element (MTE), downstream core promoter element (DPE), downstream core element (DCE), although core promoters lacking these elements are known in the art.

A promoter of the disclosure can be a Polymerase III (Pol III) promoter. Pol III transcribes DNA to synthesize small ribosomal RNAs such as the 5S rRNA, tRNAs, and other small RNAs. Representative Pol III promoters use internal control sequences (sequences within the transcribed section of the gene) to support transcription, although upstream elements such as the TATA box are also sometimes used. All Pol III promoters are envisaged as within the scope of the instant disclosure.

The term “enhancer” refers to regulatory DNA sequences that, when bound by specific proteins called transcription factors, regulate the expression of an associated gene. Enhancers may be located in the intron of the gene, or 5′ or 3′ of the coding sequence of the gene. Enhancers may be proximal to the gene (i.e., within a few tens or hundreds of base pairs (bp) of the promoter), or may be located distal to the gene (i.e., thousands of bp, hundreds of thousands of bp, or even millions of bp away from the promoter). A single gene may be regulated by more than one enhancer, all of which are envisaged as within the scope of the instant disclosure.

As used herein, a “post-transcriptional regulatory element (PRE),” such as a hepatitis PRE, refers to a DNA sequence that, when transcribed creates a tertiary structure capable of exhibiting post-transcriptional activity to enhance or promote expression of an associated gene operably linked thereto.

As used herein, a “post-transcriptional regulatory element (PTRE),” such as a hepatitis PTRE, refers to a DNA sequence that, when transcribed creates a tertiary structure capable of exhibiting post-transcriptional activity to enhance or promote expression of an associated gene operably linked thereto.

“Recombinant,” as used herein, means that a particular nucleic acid (DNA or RNA) is the product of various combinations of cloning, restriction, and/or ligation steps resulting in a construct having a structural coding or non-coding sequence distinguishable from endogenous nucleic acids found in natural systems. Generally, DNA sequences encoding the structural coding sequence can be assembled from cDNA fragments and short oligonucleotide linkers, or from a series of synthetic oligonucleotides, to provide a synthetic nucleic acid which is capable of being expressed from a recombinant transcriptional unit contained in a cell or in a cell-free transcription and translation system. Such sequences can be provided in the form of an open reading frame uninterrupted by internal non-translated sequences, or introns, which are typically present in eukaryotic genes. Genomic DNA comprising the relevant sequences can also be used in the formation of a recombinant gene or transcriptional unit. Sequences of non-translated DNA may be present 5′ or 3′ from the open reading frame, where such sequences do not interfere with manipulation or expression of the coding regions, and may indeed act to modulate production of a desired product by various mechanisms (see “enhancers” and “promoters”, above).

The term “recombinant polynucleotide” or “recombinant nucleic acid” refers to one which is not naturally occurring, e.g., is made by the artificial combination of two otherwise separated segments of sequence through human intervention. This artificial combination is often accomplished by either chemical synthesis means, or by the artificial manipulation of isolated segments of nucleic acids, e.g., by genetic engineering techniques. Such is usually done to replace a codon with a redundant codon encoding the same or a conservative amino acid, while typically introducing or removing a sequence recognition site. Alternatively, it is performed to join together nucleic acid segments of desired functions to generate a desired combination of functions. This artificial combination is often accomplished by either chemical synthesis means, or by the artificial manipulation of isolated segments of nucleic acids, e.g., by genetic engineering techniques.

Similarly, the term “recombinant polypeptide” or “recombinant protein” refers to a polypeptide or protein which is not naturally occurring, e.g., is made by the artificial combination of two otherwise separated segments of amino sequence through human intervention. Thus, e.g., a protein that comprises a heterologous amino acid sequence is recombinant.

As used herein, the term “contacting” means establishing a physical connection between two or more entities. For example, contacting a target nucleic acid with a guide nucleic acid means that the target nucleic acid and the guide nucleic acid are made to share a physical connection; e.g., can hybridize if the sequences share sequence similarity.

“Dissociation constant”, or “K_{d}”, are used interchangeably and mean the affinity between a ligand “L” and a protein “P”; i.e., how tightly a ligand binds to a particular protein. It can be calculated using the formula K_{d}=[L] [P]/[LP], where [P], [L] and [LP] represent molar concentrations of the protein, ligand and complex, respectively.

The disclosure provides systems and methods useful for editing a target nucleic acid sequence. As used herein “editing” is used interchangeably with “modifying” and includes but is not limited to cleaving, nicking, deleting, knocking in, knocking out, and the like.

By “cleavage” it is meant the breakage of the covalent backbone of a target nucleic acid molecule (e.g., RNA, DNA). Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events.

The term “knock-out” refers to the elimination of a gene or the expression of a gene. For example, a gene can be knocked out by either a deletion or an addition of a nucleotide sequence that leads to a disruption of the reading frame. As another example, a gene may be knocked out by replacing a part of the gene with an irrelevant sequence. The term “knock-down” as used herein refers to reduction in the expression of a gene or its gene product(s). As a result of a gene knock-down, the protein activity or function may be attenuated or the protein levels may be reduced or eliminated.

As used herein, “homology-directed repair” (HDR) refers to the form of DNA repair that takes place during repair of double-strand breaks in cells. This process requires nucleotide sequence homology, and uses a donor template to repair or knock-out a target DNA, and leads to the transfer of genetic information from the donor to the target. Homology-directed repair can result in an alteration of the sequence of the target sequence by insertion, deletion, or mutation if the donor template differs from the target DNA sequence and part or all of the sequence of the donor template is incorporated into the target DNA.

As used herein, “non-homologous end joining” (NHEJ) refers to the repair of double-strand breaks in DNA by direct ligation of the break ends to one another without the need for a homologous template (in contrast to homology-directed repair, which requires a homologous sequence to guide repair). NHEJ often results in the loss (deletion) of nucleotide sequence near the site of the double-strand break.

As used herein “micro-homology mediated end joining” (MMEJ) refers to a mutagenic DSB repair mechanism, which always associates with deletions flanking the break sites without the need for a homologous template (in contrast to homology-directed repair, which requires a homologous sequence to guide repair). MMEJ often results in the loss (deletion) of nucleotide sequence near the site of the double-strand break. A polynucleotide or polypeptide has a certain percent “sequence similarity” or “sequence identity” to another polynucleotide or polypeptide, meaning that, when aligned, that percentage of bases or amino acids are the same, and in the same relative position, when comparing the two sequences. Sequence similarity (sometimes referred to as percent similarity, percent identity, or homology) can be determined in a number of different manners. To determine sequence similarity, sequences can be aligned using the methods and computer programs that are known in the art, including BLAST, available over the world wide web at ncbi.nlm.nih.gov/BLAST. Percent complementarity between particular stretches of nucleic acid sequences within nucleic acids can be determined using any convenient method. Example methods include BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), e.g., using default settings, which uses the algorithm of Smith and Waterman (Adv. Appl. Math., 1981, 2, 482-489).

The terms “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones. The term includes fusion proteins, including, but not limited to, fusion proteins with a heterologous amino acid sequence.

A “vector” or “expression vector” is a replicon, such as plasmid, phage, virus, virus-like particle or cosmid, to which another DNA segment, i.e., an “insert”, may be attached so as to bring about the replication or expression of the attached segment in a cell.

The term “naturally-occurring” or “unmodified” or “wild type” as used herein as applied to a nucleic acid, a polypeptide, a cell, or an organism, refers to a nucleic acid, polypeptide, cell, or organism that is found in nature.

As used herein, a “mutation” refers to an insertion, deletion, substitution, duplication, or inversion of one or more amino acids or nucleotides as compared to a wild-type or reference amino acid sequence or to a wild-type or reference nucleotide sequence.

As used herein the term “isolated” is meant to describe a polynucleotide, a polypeptide, or a cell that is in an environment different from that in which the polynucleotide, the polypeptide, or the cell naturally occurs. An isolated genetically modified host cell may be present in a mixed population of genetically modified host cells.

A “host cell,” as used herein, denotes a eukaryotic cell, a prokaryotic cell, or a cell from a multicellular organism (e.g., in a cell line), which eukaryotic or prokaryotic cells are used as recipients for a nucleic acid (e.g., an expression vector), and include the progeny of the original cell which has been genetically modified by the nucleic acid. It is understood that the progeny of a single cell may not necessarily be completely identical in morphology or in genomic or total DNA complement as the original parent, due to natural, accidental, or deliberate mutation. A “recombinant host cell” (also referred to as a “genetically modified host cell”) is a host cell into which has been introduced a heterologous nucleic acid, e.g., an expression vector.

A “target cell marker” refers to a molecule expressed by a target cell including but not limited to cell-surface receptors, cytokine receptors, antigens, tumor-associated antigens, glycoproteins, oligonucleotides, enzymatic substrates, antigenic determinants, or binding sites that may be present in the on the surface of a target tissue or cell that may serve as ligands for an antibody fragment or glycoprotein tropism factor.

The term “conservative amino acid substitution” refers to the interchangeability in proteins of amino acid residues having similar side chains. For example, a group of amino acids having aliphatic side chains consists of glycine, alanine, valine, leucine, and isoleucine; a group of amino acids having aliphatic-hydroxyl side chains consists of serine and threonine; a group of amino acids having amide-containing side chains consists of asparagine and glutamine; a group of amino acids having aromatic side chains consists of phenylalanine, tyrosine, and tryptophan; a group of amino acids having basic side chains consists of lysine, arginine, and histidine; and a group of amino acids having sulfur-containing side chains consists of cysteine and methionine. Exemplary conservative amino acid substitution groups are: valine-leucine-isoleucine, phenylalanine-tyrosine, lysine-arginine, alanine-valine, and asparagine-glutamine.

The term “antibody,” as used herein, encompasses various antibody structures, including but not limited to monoclonal antibodies, polyclonal antibodies, multispecific antibodies (e.g., bispecific antibodies), nanobodies, single domain antibodies such as VHH antibodies, and antibody fragments so long as they exhibit the desired antigen-binding activity or immunological activity. Antibodies represent a large family of molecules that include several types of molecules, such as IgD, IgG, IgA, IgM and IgE.

An “antibody fragment” refers to a molecule other than an intact antibody that comprises a portion of an intact antibody and that binds the antigen to which the intact antibody binds. Examples of antibody fragments include but are not limited to Fv, Fab, Fab′, Fab′-SH, F(ab′)2, diabodies, single chain diabodies, linear antibodies, a single domain antibody, a single domain camelid antibody, single-chain variable fragment (scFv) antibody molecules, and multispecific antibodies formed from antibody fragments.

As used herein, “treatment” or “treating,” are used interchangeably herein and refer to an approach for obtaining beneficial or desired results, including but not limited to a therapeutic benefit and/or a prophylactic benefit. By therapeutic benefit is meant eradication or amelioration of the underlying disorder or disease being treated. A therapeutic benefit can also be achieved with the eradication or amelioration of one or more of the symptoms or an improvement in one or more clinical parameters associated with the underlying disease such that an improvement is observed in the subject, notwithstanding that the subject may still be afflicted with the underlying disorder.

The terms “therapeutically effective amount” and “therapeutically effective dose”, as used herein, refer to an amount of a drug or a biologic, alone or as a part of a composition, that is capable of having any detectable, beneficial effect on any symptom, aspect, measured parameter or characteristics of a disease state or condition when administered in one or repeated doses to a subject such as a human or an experimental animal. Such effect need not be absolute to be beneficial.

As used herein, “administering” means a method of giving a dosage of a compound (e.g., a composition of the disclosure) or a composition (e.g., a pharmaceutical composition) to a subject.

A “subject” is a mammal. Mammals include, but are not limited to, domesticated animals, non-human primates, humans, dogs, rabbits, mice, rats and other rodents.

All publications, patents, and patent applications mentioned in this specification are herein incorporated by reference to the same extent as if each individual publication, patent, or patent application was specifically and individually indicated to be incorporated by reference.

### I. General Methods

The practice of the present invention employs, unless otherwise indicated, conventional techniques of immunology, biochemistry, chemistry, molecular biology, microbiology, cell biology, genomics and recombinant DNA, which can be found in such standard textbooks as Molecular Cloning: A Laboratory Manual, 3rd Ed. (Sambrook et al., Harbor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference.

Where a range of values is provided, it is understood that endpoints are included and that each intervening value, to the tenth of the unit of the lower limit unless the context clearly dictates otherwise, between the upper and lower limit of that range and any other stated or intervening value in that stated range, is encompassed. The upper and lower limits of these smaller ranges may independently be included in the smaller ranges, and are also encompassed, subject to any specifically excluded limit in the stated range. Where the stated range includes one or both of the limits, ranges excluding either or both of those included limits are also included.

Unless defined otherwise, all technical and scientific terms used herein have the same meaning as commonly understood by one of ordinary skill in the art to which this invention belongs. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise.

It will be appreciated that certain features of the disclosure, which are, for clarity, described in the context of separate embodiments, may also be provided in combination in a single embodiment. In other cases, various features of the disclosure, which are, for brevity, described in the context of a single embodiment, may also be provided separately or in any suitable sub-combination. It is intended that all combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

### II. AAV Vectors

In a first aspect, the present disclosure relates to AAV vectors optimized for the expression and delivery of CRISPR nucleases to target cells and/or tissues for genetic editing.

Wild-type AAV is a small, single-stranded DNA virus belonging to the parvovirus family. The wild-type AAV genome is made up of two genes that encode four replication proteins and three capsid proteins, respectively, and is flanked on either side by inverted terminal repeats (ITRs) having 130-145 nucleotides that fold into a hairpin shape important for replication. The virion is composed of three capsid proteins, Vp1, Vp2, and Vp3, produced in a 1:1:10 ratio from the same open reading frame but from differential splicing (Vp1) and alternative translational start sites (Vp2 and Vp3, respectively). The cap gene produces an additional, non-structural protein called the Assembly-Activating Protein (AAP). This protein is produced from ORF2 and is essential for the capsid-assembly process. The capsid forms a supramolecular assembly of approximately 60 individual capsid protein subunits into a non-enveloped, T-1 icosahedral lattice capable of protecting the AAV genome.

Being naturally replication-defective and capable of transducing nearly every cell type in the human body, AAV represents a suitable vector for therapeutic use in gene therapy or vaccine delivery. Typically, when producing a recombinant AAV vector, the sequence between the two ITRs is replaced with one or more sequences of interest (e.g., a transgene), and the Rep and Cap sequences are provided in trans, making the ITRs the only viral DNA that remains in the vector. The resulting recombinant AAV vector genome construct comprises two cis-acting 130 to 145-nucleotide ITRs flanking an expression cassette encoding the transgene sequences of interest, providing at least 4.7 kb or more for packaging of foreign DNA that can include a transgene, one or more promoters and accessory elements, such that the total size of the vector is below 5 to 5.2 kb, which is compatible with packaging within the AAV capsid (it being understood that as the size of the construct exceeds this threshold, the packaging efficiency of the vector decreases). The transgene may be used to correct or ameliorate gene deficiencies in the cells of a subject. In the context of CRISPR-mediated gene editing, however, the size limitation of the expression cassette is a challenge for most CRISPR systems, given the large size of the nucleases.

The present disclosure provides polynucleotides for production of AAV transgene plasmids as well as for the production of AAV viral vectors. In some embodiments, the polynucleotides comprise sequences encoding a first adeno-associated virus (AAV) 5′ inverted terminal repeat (ITR) sequence, a second AAV 3′ ITR sequence, a CRISPR nuclease, a first guide RNA (gRNA), one or more promoters and, optionally, accessory elements; all encompassed in a single expression cassette encoded by a single polynucleotide capable of being incorporated into a single AAV viral particle. In other embodiments, the polynucleotides comprise sequences encoding a first 5′ AAV ITR sequence, a second 3′ AAV ITR sequence, a CRISPR nuclease, a first gRNA, a first promoter, a second promoter, and, optionally, one or more accessory elements.

The promoter and accessory elements can be operably linked to a transgene, e.g. the CRISPR protein and/or gRNA, in a manner which permits its transcription, translation and/or expression in a cell transfected with the AAV vector of the embodiments. As used herein, “operably linked” sequences include both accessory element sequences that are contiguous with the gene of interest and accessory element sequences that are at a distance to control the gene of interest. In some embodiments, the CRISPR protein and the first gRNA are under the control of, and operably linked to, a first promoter. In other embodiments, the CRISPR protein is under the control of and operably linked to a first promoter and the first gRNA is under the control of and operably linked to a second promoter.

In some embodiments, the disclosure provides accessory elements for inclusion in the AAV vector that include, but are not limited to sequences that control transcription initiation, termination, promoters, enhancer elements, RNA processing signal sequences, enhancer elements, sequences that stabilize cytoplasmic mRNA, sequences that enhance translation efficiency (i.e., Kozak consensus sequence), an intron, a post-transcriptional regulatory element (PTRE), a nuclear localization signal (NLS), a deaminase, a DNA glycosylase inhibitor, a second guide RNA, a stimulator of CRISPR-mediated homology-directed repair, and an activator or repressor of transcription.

By “adeno-associated virus inverted terminal repeats” or “AAV ITRs” is meant the art recognized regions found at each end of the AAV genome which function together in cis as origins of DNA replication and as packaging signals for the virus. AAV ITRs, together with the AAV rep coding region, provide for the efficient excision and rescue from, and integration of a nucleotide sequence interposed between two flanking ITRs into a mammalian cell genome.

The nucleotide sequences of AAV ITR regions are known. See, for example Kotin, R. M. (1994) Human Gene Therapy 5:793-801; Berns, K. I. “Parvoviridae and their Replication” in Fundamental Virology, 2nd Edition, (B. N. Fields and D. M. Knipe, eds.). As used herein, an AAV ITR need not have the wild-type nucleotide sequence depicted, but may be altered, e.g., by the insertion, deletion or substitution of nucleotides. Additionally, the AAV ITR may be derived from any of several AAV serotypes, including without limitation, AAV1, AAV2, AAV3, AAV4, AAV5, AAV6, AAV7, AAV8, AAV9, AAV10, AAV11, AAV12, AAV 44.9, AAV-Rh74, and AAVRh10, and modified capsids of these serotypes. Furthermore, 5′ and 3′ ITRs which flank a selected nucleotide sequence in an AAV vector need not necessarily be identical or derived from the same AAV serotype or isolate, so long as they function as intended, i.e., to allow for excision and rescue of the sequence of interest from a host cell genome or vector, and to allow integration of the heterologous sequence into the recipient cell genome when AAV Rep gene products are present in the cell. Use of AAV serotypes for integration of heterologous sequences into a host cell is known in the art (see, e.g., WO2018195555A1 and US20180258424A1, incorporated by reference herein). In one particular embodiment, the ITRs are derived from serotype AAV1. In another particular embodiment, the ITRs are derived from serotype AAV2, including the 5′ ITR having sequence CCTGCAGGCAGCTGCGCGCTCGCTCGCTCACTGAGGCCGCCCGGGCGTCGGGCGAC CTTTGGTCGCCCGGCCTCAGTGAGCGAGCGAGCGCGCAGAGAGGGAGTGGCCAACT CCATCACTAGGGGTTCCT (SEQ ID NO: 40557) and the 3′ ITR having sequence AGGAACCCCTAGTGATGGAGTTGGCCACTCCCTCTCTGCGCGCTCGCTCGCTCACTG AGGCCGGGCGACCAAAGGTCGCCCGACGCCCGGGCTTTGCCCGGGCGGCCTCAGTG AGCGAGCGAGCGCGCAGCTGCCTGCAGG (SEQ ID NO: 40576).

By “AAV rep coding region” is meant the region of the AAV genome which encodes the replication proteins Rep 78, Rep 68, Rep 52 and Rep 40. These Rep expression products have been shown to possess many functions, including recognition, binding and nicking of the AAV origin of DNA replication, DNA helicase activity and modulation of transcription from AAV (or other heterologous) promoters. The Rep expression products are collectively required for replicating the AAV genome.

By “AAV cap coding region” is meant the region of the AAV genome which encodes the capsid proteins VP1, VP2, and VP3, or functional homologues thereof. These Cap expression products supply the packaging functions which are collectively required for packaging the viral genome.

In some embodiments, the AAV vector is of serotype 9 or of serotype 6, which have been demonstrated to effectively deliver polynucleotides to motor neurons and glia throughout the spinal cord in preclinical models of Amyotrophic lateral sclerosis (ALS) (Foust, K D. et al. Therapeutic AAV9-mediated suppression of mutant RHO slows disease progression and extends survival in models of inherited ALS. Mol Ther. 21(12):2148 (2013)). In some embodiments, the methods provide use of AAV9 or AAV6 for targeting of neurons via intraparenchymal brain injection. In some embodiments, the methods provide use of AAV9 for intravenous administering of the vector wherein the AAV9 has the ability to penetrate the blood-brain barrier and drive gene expression in the nervous system via both neuronal and glial tropism of the vector. In other embodiments, the AAV vector is of serotype 8, which have been demonstrated to effectively deliver polynucleotides to retinal cells.

In some embodiments, the one or more accessory elements are selected from the group consisting of a poly(A) signal, a gene enhancer element, an intron, a posttranscriptional regulatory element (PTRE), a nuclear localization signal (NLS), a deaminase, a DNA glycosylase inhibitor, a third promoter, a second guide RNA (targeting a different or overlapping segment of the target nucleic acid), a stimulator of CRISPR-mediated homology-directed repair, and an activator or repressor of transcription. In some cases, the PTRE is selected from the group consisting of cytomegalovirus immediate/early intronA, hepatitis B virus PRE (HPRE), Woodchuck Hepatitis virus PRE (WPRE), and 5′ untranslated region (UTR) of human heat shock protein 70 mRNA (Hsp70).

In the foregoing, the one or more accessory elements are operably linked to the CRISPR protein. It has been discovered that the inclusion of the accessory element(s) in the polynucleotide of the AAV construct can enhance the expression, binding, activity, or performance of the CRISPR protein as compared to the CRISPR protein in the absence of said accessory element in an AAV construct. In one embodiment, the inclusion of the one or more accessory elements results in an increase in editing of a target nucleic acid by the CRISPR protein in an in vitro assay of at least about 10%, at least about 20%, at least about 30%, at least about 40%, at least about 50%, at least about 60%, at least about 70%, at least about 80%, at least about 90%, at least about 100%, at least about 1500%, at least about 200%, or at least about 300% as compared to the CRISPR protein in the absence of said accessory element in an AAV construct.

In a feature of the AAV vectors of the present disclosure, it has been discovered that utilization of certain Class 2 CRISPR systems of smaller size permit the inclusion of additional sequence space in the polynucleotides used in the making of the AAV vectors that can be utilized for the remaining components of the transgene, as described herein. In some embodiments, the Class 2 CRISPR system comprises a Type V protein selected from the group consisting of Cas12a, Cas12b, Cas12c, Cas12d (CasY), Cas12j and CasX, and the associated guide RNA of the respective system. In a particular embodiment, the CRISPR protein is a CasX, wherein the CasX comprises a sequence selected from the group consisting of SEQ ID NOS: 1-3 and SEQ ID NOS: 49-160, 40208-40369 and 40828-40912 as listed in Table 3, or a sequence having at least about 85%, at least about 90%, at least about 91%, at least about 92%, at least about 93%, at least about 94%, at least about 95%, at least about 96%, at least about 97%, at least about 98%, or at least about 99% sequence identity thereto. In a particular embodiment, the CRISPR protein is a CasX, wherein the CasX comprises a sequence selected from the group consisting of the sequences of SEQ ID NOS: 1-3 and SEQ ID NOS: 49-160 and 40208-40369 and 40828-40912 as listed in Table 3. In some embodiments, the gRNA comprises a scaffold sequence selected from the group consisting of SEQ ID NOS: 2101-2285, 39981-40026, 40913-40958, and 41817 as set forth in Table 2, or a sequence having at least 85%, at least 90%, at least 95%, at least 95%, at least 96%, at least 97%, at least 98% identity thereto. In a particular embodiment, the gRNA comprises a sequence selected from the group of sequences of SEQ ID NOS: 2101-2285, 39981-40026, 40913-40958, and 41817 as set forth in Table 2. In the foregoing embodiments, the gRNA further comprises a targeting sequence complementary to a target nucleic acid to be modified, wherein the targeting sequence has at least 15 to 20 nucleotides. The CasX protein and gRNA component embodiments contemplated for incorporation into the AAV vectors of the disclosure are described more fully, below.

As described, supra, the smaller size of the Class 2, Type V proteins and gRNA contemplated for inclusion in the AAV constructs permit inclusion of additional or larger components that can be packaged into a single AAV particle. In some embodiments, the polynucleotide encoding the CRISPR protein sequence and the gRNA sequence are less than about 3100, about 3090, about 3080, about 3070, about 3060, about 3050, or less than about 3040 nucleotides in length. In other embodiments, the polynucleotide encoding the CRISPR protein sequence and the gRNA sequence are less than about 3040 to about 3100 nucleotides in combined length. Thus, in light of the total length of the expression cassette that can be packaged into an AAV particle, in some embodiments, the polynucleotide sequences of the first promoter and the at least one accessory element have greater than at least about 1300, at least about 1350, at least about 1360, at least about 1370, at least about 1380, at least about 1390, at least about 1400, at least about 1500, at least about 1600 nucleotides, at least 1650, at least about 1700, at least about 1750, at least about 1800, at least about 1850, or at least about 1900 nucleotides in combined length. In other embodiments, the polynucleotide sequences of the first promoter and the at least one accessory element have greater than at least about 1300 to at least about 1900 nucleotides in combined length. In one embodiment, the polynucleotide sequences of the first promoter and the at least one accessory element have greater than 1314 nucleotides in combined length. In another embodiment, the polynucleotide sequences of the first promoter and the at least one accessory element have greater than 1381 nucleotides in combined length. In other embodiments, the polynucleotide sequences of the first promoter, the second promoter and the at least one accessory element have greater than at least about 1300, at least about 1350, at least about 1360, at least about 1370, at least about 1380, at least about 1390, at least about 1400, at least about 1500, at least about 1600 nucleotides, at least 1650, at least about 1700, at least about 1750, at least about 1800, at least about 1850, or at least about 1900 nucleotides in combined length. In other embodiments, the polynucleotide sequences of the first promoter, the second promoter and the at least one accessory element have greater than at least about 1300 to at least about 1900 nucleotides in combined length. In one embodiment, the polynucleotide sequences of the first promoter, the second promoter, and the at least one accessory element have greater than 1314 nucleotides in combined length. In other embodiments, the polynucleotide sequences of the first promoter, the second promoter, and the at least one accessory element have greater than 1381 nucleotides in combined length. In still other embodiments, the polynucleotide sequences of the first promoter, the second promoter, and the two or more accessory elements have greater than at least about 1300, at least about 1350, at least about 1360, at least about 1370, at least about 1380, at least about 1390, at least about 1400, at least about 1500, at least about 1600 nucleotides, at least 1650, at least about 1700, at least about 1750, at least about 1800, at least about 1850, or at least about 1900 nucleotides in combined length. In other embodiments, the polynucleotide sequences of the first promoter, the second promoter, and the two or more accessory elements have greater than at least about 1300 to at least about 1900 nucleotides in combined length. In one embodiment, the polynucleotide sequences of the first promoter, the second promoter, and the two or more accessory elements have greater than 1314 nucleotides in combined length. In another embodiment, the polynucleotide sequences of the first promoter, the second promoter, and the two or more accessory elements have greater than 1381 nucleotides in combined length.

In some embodiments, the present disclosure provides a polynucleotide comprising a first adeno-associated virus (AAV) inverted terminal repeat (ITR) sequence, a second AAV ITR sequence, a first promoter sequence, a sequence encoding a CRISPR protein, a second promoter sequence, a sequence encoding at least a first guide RNA (gRNA), and one or more accessory element sequences, wherein at least 25%, 26%, 27%, 28%, 29%, 30%, 31%, 32%, 33%, 34%, or 35% or more of the nucleotides of the polynucleotide sequence comprise the first and second promoters and the one or more accessory element sequences in combined length. In other embodiments, the present disclosure provides a polynucleotide comprising a first adeno-associated virus (AAV) inverted terminal repeat (ITR) sequence, a second AAV ITR sequence, a first promoter sequence, a sequence encoding a CRISPR protein, a second promoter sequence, a sequence encoding a first guide RNA (gRNA), a third promoter sequence, a sequence encoding a second gRNA, and one or more accessory element sequences, wherein at least 25%, 26%, 27%, 28%, 29%, 30%, 31%, 32%, 33%, 34%, or 35% or more of the nucleotides of the polynucleotide sequence comprise the first, second, and third promoters and the one or more accessory element sequences in combined length. As detailed in the Examples, it has been discovered that the ability to devote more of the total polynucleotide of the expression cassette of an AAV transgene to the promoters, a second gRNA, and/or the accessory elements results in enhanced expression of and/or performance of the CRISPR protein and gRNA, when expressed in the target host cell; either in an in vitro assay or in vivo in a subject. In some embodiments, the use of alternative or longer promoters and/or accessory elements (e.g., poly(A) signal, a gene enhancer element, an intron, a posttranscriptional regulatory element (PTRE), a nuclear localization signal (NLS), a deaminase, a DNA glycosylase inhibitor, a stimulator of CRISPR-mediated homology-directed repair, and an activator or repressor of transcription) in the AAV polynucleotides and resulting AAV vectors results in an increase in editing of a target nucleic acid of at least about 10%, at least about 20%, at least about 30%, at least about 40%, at least about 50%, at least about 60%, at least about 70%, at least about 80%, at least about 90%, at least about 100%, at least about 1500%, at least about 200%, or at least about 300% when the AAV is assessed in an in vitro assay compared to a construct not having the alternative or longer promoters and/or accessory elements. In one embodiment, the first promoter sequence of the polynucleotide has at least about 200, at least about 300, at least about 400, at least about 500, at least about 600, at least about 700, or at least about 800 nucleotides. In another embodiment, the second promoter sequence of the polynucleotide has at least about 200, at least about 300, at least about 400, at least about 500, at least about 600, at least about 700, or at least about 800 nucleotides. Embodiments of the promoters are described more fully, below.

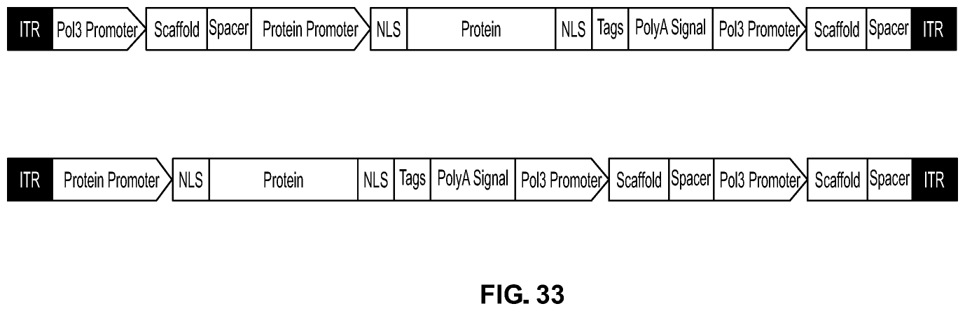

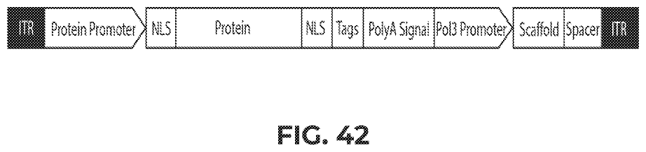

In some embodiments, the present disclosure provides a polynucleotide, wherein the polynucleotide comprises one or more sequences selected from the group of sequences set forth in Tables 8-10, 12, 13, 17-22 and 24-27, or a sequence having at least 85%, at least 90%, at least 95%, at least 95%, at least 96%, at least 97%, at least 98%, or at least 99% identity thereto. In another embodiment, the present disclosure provides a polynucleotide, wherein the polynucleotide comprises a sequence selected from the group of sequences set forth in Tables 8-10, 12, 13, and 17-23 and 24-27. In some embodiments, the polynucleotide sequence differs from those set forth in Tables 8-10, 12, 13, and 17-22 and 24-26 only in the selection of the targeting sequences of the gRNA or gRNAs encoded by the polynucleotide, wherein the targeting sequence is a sequence having 15 to 30 nucleotides capable of hybridizing with the sequence of a target nucleic acid. In a particular embodiment of the foregoing, the targeting sequence is selected from the group of sequences set forth in Table 27. In some embodiments, the present disclosure provides a polynucleotide of any of the embodiments described herein, wherein the polynucleotide has the configuration of a construct of FIG. 24, FIGS. 33-35, or FIG. 42.

In some embodiments, the present disclosure provides a polynucleotide for use in the making of an AAV vector, wherein the polynucleotide comprises one or more sequences selected from the group of sequences set forth in Tables 8-10, 12, 13, and 17-22 and 24-27, or a sequence having at least 85%, at least 90%, at least 95%, at least 95%, at least 96%, at least 97%, at least 98%, or at least 99% identity thereto. In another embodiment, the present disclosure provides a polynucleotide for use in the making of an AAV vector, wherein the polynucleotide comprises a sequence selected from the group of sequences set forth in Tables 8-10, 12, 13, 17-22 and 24-27. In some embodiments, the polynucleotide sequence differs from those set forth in Tables 8-10, 12, 13, 17-22 and 24-26 only in the selection of the targeting sequences of the gRNA or gRNAs encoded by the polynucleotide, wherein the targeting sequence is a sequence having 15 to 30 nucleotides and is capable of hybridizing with the sequence of a target nucleic acid to be modified. In a particular embodiment of the foregoing, the targeting sequence is selected from the group of sequences set forth in Table 27. In some embodiments, the present disclosure provides a polynucleotide of any of the embodiments described herein for use in the making of an AAV vector, wherein the polynucleotide has the configuration of a construct of FIG. 24, FIGS. 33-35, or FIG. 42.

### III. Guide Nucleic Acids of the AAV Systems

In some embodiments, the disclosure relates to specifically-designed guide ribonucleic acids (gRNA) utilized in the AAV systems that have utility in genome editing of a target nucleic acid in a cell. The present disclosure provides specifically-designed gRNAs with targeting sequences that are complementary to (and are therefore able to hybridize with) the target nucleic acid as a component of the gene editing AAV systems. It is envisioned that in some embodiments, multiple gRNAs (e.g., multiple gRNAs) are delivered in the AAV system for the modification of a target nucleic acid. For example, a pair of gRNAs with targeting sequences to different or overlapping regions of the target nucleic acid sequence can be used, when each is complexed with a CRISPR nuclease, in order to bind and cleave at two different or overlapping sites within the gene, which is then edited by non-homologous end joining (NHEJ), homology-directed repair (HDR), homology-independent targeted integration (HITI), micro-homology mediated end joining (MMEJ), single strand annealing (SSA) or base excision repair (BER).

In some embodiments, the disclosure provides gRNAs utilized in the systems that have utility in genome editing a gene in a eukaryotic cell. In a particular embodiment, the gRNA of the systems are capable of forming a complex with a CRISPR nuclease; a ribonucleoprotein (RNP) complex, described more fully, below.

a. Reference gRNA and gRNA Variants

In some embodiments, a gRNA of the present disclosure comprises a sequence of a naturally-occurring guide RNA (a “reference gRNA”). In some embodiments, a reference gRNA of the disclosure may be subjected to one or more mutagenesis methods, such as the mutagenesis methods described herein, which may include Deep Mutational Evolution (DME), deep mutational scanning (DMS), error prone PCR, cassette mutagenesis, random mutagenesis, staggered extension PCR, gene shuffling, or domain swapping (as described herein, as well as in WO2020247883A2, incorporated by reference herein), in order to generate one or more variants (referred to herein as “gRNA variant”) with enhanced or varied properties relative to the reference gRNA. gRNA variants also include variants comprising one or more exogenous sequences, for example fused to either the 5′ or 3′ end, or inserted internally. The activity of reference gRNAs or the variant from which it was derived may be used as a benchmark against which the activity of gRNA variants are compared, thereby measuring improvements in function or other characteristics of the gRNA variants. In other embodiments, a reference gRNA or a gRNA variant may be subjected to one or more deliberate, specifically-targeted mutations in order to produce a gRNA variant; for example a rationally designed variant.

In some embodiments, the guide is a ribonucleic acid molecule (“gRNA”), and in other embodiments, the guide is a chimera, and comprises both DNA and RNA.

The gRNAs of the disclosure comprise two segments; a targeting sequence and a protein-binding segment. The targeting segment of a gRNA includes a nucleotide sequence (referred to interchangeably as a guide sequence, a spacer, a targeting sequence, or a targeting region) that is complementary to, and therefore can hybridize with, a specific sequence (a target site) within the target nucleic acid (e.g., a target ssRNA, a target ssDNA, the complementary strand of a double stranded target DNA, etc.), described more fully below. The targeting sequence of a gRNA is capable of binding to a target nucleic acid sequence, including a coding sequence, a complement of a coding sequence, a non-coding sequence, and to accessory elements. The protein-binding segment (or “protein-binding sequence”) interacts with (e.g., binds to) a CasX protein as a complex, forming an RNP (described more fully, below). The protein-binding segment is alternatively referred to herein as a “scaffold”, which is comprised of several regions, described more fully, below.

In the case of a dual guide RNA (dgRNA), the targeter and the activator portions each have a duplex-forming segment, where the duplex forming segment of the targeter and the duplex-forming segment of the activator have complementarity with one another and hybridize to one another to form a double stranded duplex (dsRNA duplex for a gRNA). When the gRNA is a gRNA, the term “targeter” or “targeter RNA” is used herein to refer to a crRNA-like molecule (crRNA: “CRISPR RNA”) of a CasX dual guide RNA (and therefore of a CasX single guide RNA when the “activator” and the “targeter” are linked together, e.g., by intervening nucleotides). The crRNA has a 5′ region that anneals with the tracrRNA followed by the nucleotides of the targeting sequence. Thus, for example, a guide RNA (dgRNA or sgRNA) comprises a guide sequence and a duplex-forming segment of a crRNA, which can also be referred to as a crRNA repeat. A corresponding tracrRNA-like molecule (activator) also comprises a duplex-forming stretch of nucleotides that forms the other half of the dsRNA duplex of the protein-binding segment of the guide RNA. Thus, a targeter and an activator, as a corresponding pair, hybridize to form a dual guide RNA, referred to herein as a “dual-molecule gRNA”, a “dgRNA”, a “double-molecule guide RNA”, or a “two-molecule guide RNA”. Site-specific binding and/or cleavage of a target nucleic acid sequence (e.g., genomic DNA) by the CasX protein can occur at one or more locations (e.g., a sequence of a target nucleic acid) determined by base-pairing complementarity between the targeting sequence of the gRNA and the target nucleic acid sequence. Thus, for example, the gRNA of the disclosure have sequences complementarity to and therefore can hybridize with the target nucleic acid that is adjacent to a sequence complementary to a TC PAM motif or a PAM sequence, such as ATC, CTC, GTC, or TTC. Because the targeting sequence of a guide sequence hybridizes with a sequence of a target nucleic acid sequence, a targeter can be modified by a user to hybridize with a specific target nucleic acid sequence, so long as the location of the PAM sequence is considered. Thus, in some cases, the sequence of a targeter may be the complement to a non-naturally occurring sequence. In other cases, the sequence of a targeter may be a naturally-occurring sequence, derived from the complement to the gene sequence to be edited. In other embodiments, the activator and targeter of the gRNA are covalently linked to one another (rather than hybridizing to one another) and comprise a single molecule, referred to herein as a “single-molecule gRNA,” “single guide RNA”, a “single-molecule guide RNA,” a “one-molecule guide RNA”, or a “sgRNA”. In some embodiments, the sgRNA includes an “activator” or a “targeter” and thus can be an “activator-RNA” and a “targeter-RNA,” respectively. In some embodiments, the gRNA is a ribonucleic acid molecule (“gRNA”), and in other embodiments, the gRNA is a chimera, and comprises both DNA and RNA. As used herein, the term gRNA cover naturally-occurring molecules, as well as sequence variants (e.g. non-naturally occurring modified nucleotides).

Collectively, the assembled gRNAs of the disclosure comprise four distinct regions, or domains: the RNA triplex, the scaffold stem, the extended stem, and the targeting sequence that, in the embodiments of the disclosure, is specific for a target nucleic acid and is located on the 3′end of the gRNA. The RNA triplex, the scaffold stem, and the extended stem, together, are referred to as the “scaffold” of the gRNA (gRNA scaffold). The gRNA scaffolds of the disclosure can comprise RNA, or RNA and DNA.

b. RNA Triplex

In some embodiments of the guide NAs provided herein (including reference sgRNAs), there is a RNA triplex, and the RNA triplex comprises the sequence of a UUU-nX(˜4-15)-UUU (SEQ ID NO: 19) stem loop that ends with an AAAG (SEQ ID NO: 40786) after 2 intervening stem loops (the scaffold stem loop and the extended stem loop), forming a pseudoknot that may also extend past the triplex into a duplex pseudoknot. The UU-UUU-AAA (SEQ ID NO: 40787) sequence of the triplex forms as a nexus between the targeting sequence, scaffold stem, and extended stem. In exemplary CasX sgRNAs, the UUU-loop-UUU region is coded for first, then the scaffold stem loop, and then the extended stem loop, which is linked by the tetraloop, and then an AAAG (SEQ ID NO: 40786) closes off the triplex before becoming the targeting sequence.

c. Scaffold Stem Loop

In some embodiments of CasX sgRNAs of the disclosure, the triplex region is followed by the scaffold stem loop. The scaffold stem loop is a region of the gRNA that is bound by CasX protein (such as a CasX variant protein). In some embodiments, the scaffold stem loop is a fairly short and stable stem loop. In some cases, the scaffold stem loop does not tolerate many changes, and requires some form of an RNA bubble. In some embodiments, the scaffold stem is necessary for CasX sgRNA function. While it is perhaps analogous to the nexus stem of Cas9 as being a critical stem loop, the scaffold stem of a CasX sgRNA, in some embodiments, has a necessary bulge (RNA bubble) that is different from many other stem loops found in CRISPR/Cas systems. In some embodiments, the presence of this bulge is conserved across sgRNA that interact with different CasX proteins. An exemplary sequence of a scaffold stem loop sequence of a gRNA comprises the sequence CCAGCGACUAUGUCGUAUGG (SEQ ID NO: 14).

d. Extended Stem Loop

In some embodiments of the CasX sgRNAs of the disclosure, the scaffold stem loop is followed by the extended stem loop. In some embodiments, the extended stem comprises a synthetic tracr and crRNA fusion that is largely unbound by the CasX protein. In some embodiments, the extended stem loop can be highly malleable. In some embodiments, a single guide gRNA is made with a GAAA (SEQ ID NO: 40788) tetraloop linker or a GAGAAA (SEQ ID NO: 40789) linker between the tracr and crRNA in the extended stem loop. In some cases, the targeter and activator of a CasX sgRNA are linked to one another by intervening nucleotides and the linker can have a length of from 3 to 20 nucleotides. In some embodiments of the CasX sgRNAs of the disclosure, the extended stem is a large 32-bp loop that sits outside of the CasX protein in the ribonucleoprotein complex. An exemplary sequence of an extended stem loop sequence of a sgRNA comprises the sequence GCGCUUAUUUAUCGGAGAGAAAUCCGAUAAAUAAGAAGC (SEQ ID NO: 15).

e. Targeting Sequence

In some embodiments of the gRNAs of the disclosure, the extended stem loop is followed by a region that forms part of the triplex, and then the targeting sequence (or “spacer”) at the 3′ end of the gRNA. The targeting sequence targets the CasX ribonucleoprotein holo complex to a specific region of the target nucleic acid sequence of the gene to be modified. Thus, for example, gRNA targeting sequences of the disclosure have sequences complementarity to, and therefore can hybridize to, a portion of a gene in a target nucleic acid in a eukaryotic cell (e.g., a eukaryotic chromosome, chromosomal sequence, etc.) as a component of the RNP when the TC PAM motif or any one of the PAM sequences TTC, ATC, GTC, or CTC is located 1 nucleotide 5′ to the non-target strand sequence complementary to the target sequence. The targeting sequence of a gRNA can be modified so that the gRNA can target a desired sequence of any desired target nucleic acid sequence, so long as the PAM sequence location is taken into consideration. In some embodiments, the gRNA scaffold is 5′ of the targeting sequence, with the targeting sequence on the 3′ end of the gRNA. In some embodiments, the PAM motif sequence recognized by the nuclease of the RNP is TC. In other embodiments, the PAM sequence recognized by the nuclease of the RNP is NTC; i.e., ATC, CTC, GTC, or TTC.

In some embodiments, the disclosure provides a gRNA wherein the targeting sequence of the gRNA is complementary to a target nucleic acid sequence of a gene to be modified. In some embodiments, the targeting sequence of the gRNA is complementary to a target nucleic acid sequence of a gene comprising one or more mutations compared to a wild-type gene sequence for purposes of editing the sequence comprising the mutations with the CasX:gRNA systems of the disclosure. In such cases, the modification effected by the CasX:gRNA system can either correct or compensate for the mutation or can knock down or knock out expression of the mutant gene product. In other embodiments, the targeting sequence of the gRNA is complementary to a target nucleic acid sequence of a wild-type gene for purposes of editing the sequence to introduce a mutation with the CasX:gRNA systems of the disclosure in order to knock-down or knock-out the gene. In some embodiments, the targeting sequence of a gRNA is designed to be specific for an exon of the gene of the target nucleic acid. In other embodiments, the targeting sequence of a gRNA is designed to be specific for an intron of the gene of the target nucleic acid. In other embodiments, the targeting sequence of the gRNA is designed to be specific for an intron-exon junction of the gene of the target nucleic acid. In other embodiments, the targeting sequence of the gRNA is designed to be specific for a regulatory element of the gene of the target nucleic acid. In some embodiments, the targeting sequence of the gRNA is designed to be complementary to a sequence comprising one or more single nucleotide polymorphisms (SNPs) in a gene of the target nucleic acid. SNPs that are within the coding sequence or within non-coding sequences are both within the scope of the instant disclosure. In other embodiments, the targeting sequence of the gRNA is designed to be complementary to a sequence of an intergenic region of the gene of the target nucleic acid.

In some embodiments, the targeting sequence is specific for a regulatory element that regulates expression of the gene product. Such regulatory elements include, but are not limited to promoter regions, enhancer regions, intergenic regions, 5′ untranslated regions (5′ UTR), 3′ untranslated regions (3′ UTR), conserved elements, and regions comprising cis-regulatory elements. The promoter region is intended to encompass nucleotides within 5 kb of the initiation point of the encoding sequence or, in the case of gene enhancer elements or conserved elements, can be thousands of bp, hundreds of thousands of bp, or even millions of bp away from the encoding sequence of the gene of the target nucleic acid. In the foregoing, the targets are those in which the encoding gene of the target is intended to be knocked out or knocked down such that the gene product is not expressed or is expressed at a lower level in a cell.

In some embodiments, the targeting sequence of a gRNA incorporated into the AAV of any of the embodiments described herein has between 14 and 35 consecutive nucleotides. In some embodiments, the targeting sequence of a gRNA has between 10 and 30 consecutive nucleotides. In some embodiments, the targeting sequence has 10, 11, 12, 13, 14, 15, 16, 17, 18, 19, 20, 21, 22, 23, 24, 25, 26, 27, 28, 29, or 30 consecutive nucleotides. In some embodiments, the targeting sequence of the gRNA consists of 20 consecutive nucleotides. In some embodiments, the targeting sequence consists of 19 consecutive nucleotides. In some embodiments, the targeting sequence consists of 18 consecutive nucleotides. In some embodiments, the targeting sequence consists of 17 consecutive nucleotides. In some embodiments, the targeting sequence consists of 16 consecutive nucleotides. In some embodiments, the targeting sequence consists of 15 consecutive nucleotides. In some embodiments, the targeting sequence has 10, 11, 12, 13, 14, 15, 16, 17, 18, 19, 20, 21, 22, 23, 24, 25, 26, 27, 28, 29, or 30 consecutive nucleotides and the targeting sequence can comprise 0 to 5, 0 to 4, 0 to 3, or 0 to 2 mismatches relative to the target nucleic acid sequence and retain sufficient binding specificity such that the RNP comprising the gRNA comprising the targeting sequence can form a complementary bond with respect to the target nucleic acid to be modified. In some embodiments, the targeting sequence of a gRNA incorporated into the AAV of any of the embodiments described herein comprises a sequence selected from the group consisting of the sequences of SEQ ID NO: 41056-41776, as set forth in Table 27, or a sequence having at least about 80%, or at least 90%, or at least 95% thereto. In some embodiments, the targeting sequence of a gRNA incorporated into the AAV of any of the embodiments described herein consists of a sequence selected from the group consisting of the sequences of SEQ ID NO: 41056-41776, as set forth in Table 27.

In some embodiments, the CasX:gRNA system comprises a first gRNA and further comprises a second (and optionally a third, fourth, fifth, or more) gRNA, wherein the second gRNA or additional gRNA has a targeting sequence complementary to a different or overlapping portion of the target nucleic acid sequence compared to the targeting sequence of the first gRNA such that multiple points in the target nucleic acid are targeted, and for example, multiple breaks are introduced in the target nucleic acid by the CasX. It will be understood that in such cases, the second or additional gRNA is complexed with an additional copy of the CasX protein. By selection of the targeting sequences of the gRNA, defined regions of the target nucleic acid sequence bracketing a mutation can be modified or edited using the CasX:gRNA systems described herein, including facilitating the insertion of a donor template or the excision of the DNA between the cleavage sites in cases, for example, where mutant repeats occur or where removal of an exon comprising mutations nevertheless results in expression of a functional gene product.

f. gRNA Scaffolds

With the exception of the targeting sequence domain, the remaining components of the gRNA are referred to herein as the scaffold. In some embodiments, the gRNA scaffolds are derived from naturally occurring sequences, described below as reference gRNA. In other embodiments, the gRNA scaffolds are variants of reference gRNA wherein mutations, insertions, deletions or domain substitutions are introduced to confer desirable properties on the gRNA.

In some embodiments, a CasX reference gRNA comprises a sequence isolated or derived from Deltaproteobacter. In some embodiments, the sequence is a CasX tracrRNA sequence. Exemplary CasX reference tracrRNA sequences isolated or derived from Deltaproteobacter may include: ACAUCUGGCGCGUUUAUUCCAUUACUUUGGAGCCAGUCCCAGCGACUAUGUCGU AUGGACGAAGCGCUUAUUUAUCGGAGA (SEQ ID NO: 22) and ACAUCUGGCGCGUUUAUUCCAUUACUUUGGAGCCAGUCCCAGCGACUAUGUCGU AUGGACGAAGCGCUUAUUUAUCGG (SEQ ID NO: 23). Exemplary crRNA sequences isolated or derived from Deltaproteobacter may comprise a sequence of CCGAUAAGUAAAACGCAUCAAAG (SEQ ID NO: 24). In some embodiments, a CasX reference gRNA comprises a sequence identical to a sequence isolated or derived from Deltaproteobacter.

In some embodiments, a CasX reference guide RNA comprises a sequence isolated or derived from Planctomycetes. In some embodiments, the sequence is a CasX tracrRNA sequence. Exemplary CasX reference tracrRNA sequences isolated or derived from Planctomycetes may include: UACUGGCGCUUUUAUCUCAUUACUUUGAGAGCCAUCACCAGCGACUAUGUCGUA UGGGUAAAGCGCUUAUUUAUCGGAGA (SEQ ID NO: 25) and UACUGGCGCUUUUAUCUCAUUACUUUGAGAGCCAUCACCAGCGACUAUGUCGUA UGGGUAAAGCGCUUAUUUAUCGG (SEQ ID NO: 26). Exemplary crRNA sequences isolated or derived from Planctomycetes may comprise a sequence of UCUCCGAUAAAUAAGAAGCAUCAAAG (SEQ ID NO: 27). In some embodiments, a CasX reference gRNA comprises a sequence identical to a sequence isolated or derived from Planctomycetes.

In some embodiments, a CasX reference gRNA comprises a sequence isolated or derived from Candidatus Sungbacteria. In some embodiments, the sequence is a CasX tracrRNA sequence. Exemplary CasX reference tracrRNA sequences isolated or derived from Candidatus Sungbacteria may comprise sequences of: GUUUACACACUCCCUCUCAUAGGGU (SEQ ID NO: 28), GUUUACACACUCCCUCUCAUGAGGU (SEQ ID NO: 11), UUUUACAUACCCCCUCUCAUGGGAU (SEQ ID NO: 12) and GUUUACACACUCCCUCUCAUGGGGG (SEQ ID NO: 13). In some embodiments, a CasX reference guide RNA comprises a sequence identical to a sequence isolated or derived from Candidatus Sungbacteria.

Table 1 provides the sequences of reference gRNA tracr, cr and scaffold sequences. In some embodiments, the disclosure provides gRNA variant sequences wherein the gRNA has a scaffold comprising a sequence having at least one nucleotide modification relative to a reference gRNA sequence having a sequence of any one of SEQ ID NOS: 4-16 of Table 1. It will be understood that in those embodiments wherein a vector comprises a DNA encoding sequence for a gRNA, or where a gRNA is a chimera of RNA and DNA, that thymine (T) bases can be substituted for the uracil (U) bases of any of the gRNA sequence embodiments described herein.

TABLE 1
Reference gRNA tracr and scaffold sequences

| SEQ ID NO. | Nucleotide Sequence |
|---|---|
| 4 | ACAUCUGGCGCGUUUAUUCCAUUACUUUGGAGCCAGUCCCAGCGACUAUGUCGUAUGGACGAAG |
| | CGCUUAUUUAUCGGAGAGAAACCGAUAAGUAAAACGCAUCAAAG |
| 5 | UACUGGCGCUUUUAUCUCAUUACUUUGAGAGCCAUCACCAGCGACUAUGUCGUAUGGGUAAAGC |
| | GCUUAUUUAUCGGAGAGAAAUCCGAUAAAUAAGAAGCAUCAAAG |
| 6 | ACAUCUGGCGCGUUUAUUCCAUUACUUUGGAGCCAGUCCCAGCGACUAUGUCGUAUGGACGAAG |
| | CGCUUAUUUAUCGGAGA |
| 7 | ACAUCUGGCGCGUUUAUUCCAUUACUUUGGAGCCAGUCCCAGCGACUAUGUCGUAUGGACGAAG |
| | CGCUUAUUUAUCGG |
| 8 | UACUGGCGCUUUUAUCUCAUUACUUUGAGAGCCAUCACCAGCGACUAUGUCGUAUGGGUAAAGC |
| | GCUUAUUUAUCGGAGA |
| 9 | UACUGGCGCUUUUAUCUCAUUACUUUGAGAGCCAUCACCAGCGACUAUGUCGUAUGGGUAAAGC |
| | GCUUAUUUAUCGG |
| 10 | GUUUACACACUCCCUCUCAUAGGGU |
| 11 | GUUUACACACUCCCUCUCAUGAGGU |
| 12 | UUUUACAUACCCCCUCUCAUGGGAU |
| 13 | GUUUACACACUCCCUCUCAUGGGGG |
| 14 | CCAGCGACUAUGUCGUAUGG |
| 15 | GCGCUUAUUUAUCGGAGAGAAAUCCGAUAAAUAAGAAGC |
| 16 | GGCGCUUUUAUCUCAUUACUUUGAGAGCCAUCACCAGCGACUAUGUCGUAUGGGUAAAGCGCUU |
| | AUUUAUCGGA |

g. gRNA Variants

In another aspect, the disclosure relates to gRNA variants, which comprise one or more modifications relative to a reference gRNA scaffold or are derived from another gRNA variant. As used herein, “scaffold” refers to all parts to the gRNA necessary for gRNA function with the exception of the spacer sequence.

In some embodiments, a gRNA variant comprises one or more nucleotide substitutions, insertions, deletions, or swapped or replaced regions relative to a reference gRNA sequence of the disclosure. In some embodiments, a mutation can occur in any region of a reference gRNA scaffold to produce a gRNA variant. In some embodiments, the scaffold of the gRNA variant sequence has at least 20%, at least 30%, at least 40%, at least 50%, at least 60%, or at least 70%, at least 80%, at least 85%, at least about 90%, at least about 95%, at least about 96%, at least about 97%, at least about 98%, or at least about 99% identity to the sequence of SEQ ID NO: 4 or SEQ ID NO: 5. In other embodiments, a gRNA variant comprises one or more nucleotide substitutions, insertions, deletions, or swapped or replaced regions relative to a gRNA variant sequence of the disclosure. In some embodiments, the scaffold of the gRNA variant sequence has at least 50%, at least 60%, or at least 70%, at least 80%, at least 85%, at least about 90%, at least about 95%, at least about 96%, at least about 97%, at least about 98%, or at least about 99% identity to the sequence of SEQ ID NO: 2238 or SEQ ID NO: 2239.

In some embodiments, a gRNA variant comprises one or more nucleotide changes within one or more regions of the reference gRNA scaffold that improve a characteristic of the reference gRNA. Exemplary regions include the RNA triplex, the pseudoknot, the scaffold stem loop, and the extended stem loop. In some cases, the variant scaffold stem further comprises a bubble. In other cases, the variant scaffold further comprises a triplex loop region. In still other cases, the variant scaffold further comprises a 5′ unstructured region. In some embodiments, the gRNA variant scaffold comprises a scaffold stem loop having at least 60% sequence identity, at least 70% sequence identity, at least 80% sequence identity, at least 90% sequence identity, at least 95% sequence identity, or at least 99% sequence identity to SEQ ID NO: 14. In some embodiments, the gRNA variant scaffold comprises a scaffold stem loop having at least 60% sequence identity to SEQ ID NO: 14. In other embodiments, the gRNA variant comprises a scaffold stem loop having the sequence of CCAGCGACUAUGUCGUAGUGG (SEQ ID NO: 32). In other embodiments, the disclosure provides a gRNA scaffold comprising, relative to SEQ ID NO: 5, a C18G substitution, a G55 insertion, a U1 deletion, and a modified extended stem loop in which the original 6 nt loop and 13 most-loop-proximal base pairs (32 nucleotides total) are replaced by a Uvsx hairpin (4 nt loop and 5 loop-proximal base pairs; 14 nucleotides total) and the loop-distal base of the extended stem was converted to a fully base-paired stem contiguous with the new Uvsx hairpin by deletion of the A99 and substitution of G65U. In the foregoing embodiment, the gRNA scaffold 174 comprises the sequence ACUGGCGCUUUUAUCUGAUUACUUUGAGAGCCAUCACCAGCGACUAUGUCGUAG UGGGUAAAGCUCCCUCUUCGGAGGGAGCAUCAAAG (SEQ ID NO: 2238).

All gRNA variants that have one or more improved characteristics, or add one or more new functions when the variant gRNA is compared to a reference gRNA described herein, are envisaged as within the scope of the disclosure. A representative example of such a gRNA variant is guide 174 (SEQ ID NO: 2238), the design of which is described in the Examples, and guide 235 (SEQ ID NO: 39987). In some embodiments, the gRNA variant adds a new function to the RNP comprising the gRNA variant. In some embodiments, the gRNA variant has an improved characteristic selected from: increased stability; increased transcription of the gRNA; increased resistance to nuclease activity; increased folding rate of the gRNA; decreased side product formation during folding; increased productive folding; increased binding affinity to a CasX protein; increased binding affinity to a target nucleic acid when complexed with a CasX protein; increased gene editing when complexed with a CasX protein; increased specificity of editing of the target nucleic acid when complexed with a CasX protein; decreased off-target editing when complexed with a CasX protein; and increased ability to utilize a greater spectrum of one or more PAM sequences, including ATC, CTC, GTC, or TTC, in the editing of target nucleic acid when complexed with a CasX protein, and any combination thereof. In some cases, the one or more of the improved characteristics of the gRNA variant is at least about 1.1 to about 100,000-fold increased relative to the reference gRNA of SEQ ID NO: 4 or SEQ ID NO: 5, or to gRNA variant 174 or 175. In other cases, the one or more improved characteristics of the gRNA variant is at least about 1.1, at least about 10, at least about 100, at least about 1000, at least about 10,000, at least about 100,000-fold or more increased relative to the reference gRNA of SEQ ID NO: 4 or SEQ ID NO: 5, or to gRNA variant 174 or 175. In other cases, the one or more of the improved characteristics of the gRNA variant is about 1.1 to 100,00-fold, about 1.1 to 10,00-fold, about 1.1 to 1,000-fold, about 1.1 to 500-fold, about 1.1 to 100-fold, about 1.1 to 50-fold, about 1.1 to 20-fold, about 10 to 100,00-fold, about 10 to 10,00-fold, about 10 to 1,000-fold, about 10 to 500-fold, about 10 to 100-fold, about 10 to 50-fold, about 10 to 20-fold, about 2 to 70-fold, about 2 to 50-fold, about 2 to 30-fold, about 2 to 20-fold, about 2 to 10-fold, about 5 to 50-fold, about 5 to 30-fold, about 5 to 10-fold, about 100 to 100,00-fold, about 100 to 10,00-fold, about 100 to 1,000-fold, about 100 to 500-fold, about 500 to 100,00-fold, about 500 to 10,00-fold, about 500 to 1,000-fold, about 500 to 750-fold, about 1,000 to 100,00-fold, about 10,000 to 100,00-fold, about 20 to 500-fold, about 20 to 250-fold, about 20 to 200-fold, about 20 to 100-fold, about 20 to 50-fold, about 50 to 10,000-fold, about 50 to 1,000-fold, about 50 to 500-fold, about 50 to 200-fold, or about 50 to 100-fold, increased relative to the reference gRNA of SEQ ID NO: 4 or SEQ ID NO: 5, or to gRNA variant 174 or 175. In other cases, the one or more improved characteristics of the gRNA variant is about 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 25-fold, 30-fold, 40-fold, 45-fold, 50-fold, 55-fold, 60-fold, 70-fold, 80-fold, 90-fold, 100-fold, 110-fold, 120-fold, 130-fold, 140-fold, 150-fold, 160-fold, 170-fold, 180-fold, 190-fold, 200-fold, 210-fold, 220-fold, 230-fold, 240-fold, 250-fold, 260-fold, 270-fold, 280-fold, 290-fold, 300-fold, 310-fold, 320-fold, 330-fold, 340-fold, 350-fold, 360-fold, 370-fold, 380-fold, 390-fold, 400-fold, 425-fold, 450-fold, 475-fold, or 500-fold increased relative to the reference gRNA of SEQ ID NO: 4 or SEQ ID NO: 5, or to gRNA variant 174 or 175.

In some embodiments, a gRNA variant can be created by subjecting a reference gRNA or a gRNA variant to a one or more mutagenesis methods, such as the mutagenesis methods described herein, below, which may include Deep Mutational Evolution (DME), deep mutational scanning (DMS), error prone PCR, cassette mutagenesis, random mutagenesis, staggered extension PCR, gene shuffling, or domain swapping, in order to generate the gRNA variants of the disclosure. The activity of reference gRNA or gRNA variant may be used as a benchmark against which the activity of gRNA variants are compared, thereby measuring improvements in function of gRNA variants. In other embodiments, a reference gRNA or gRNA variant may be subjected to one or more deliberate, targeted mutations, substitutions, or domain swaps in order to produce a gRNA variant, for example a rationally designed variant. Exemplary gRNA variants produced by such methods are described in the Examples and representative sequences of gRNA scaffolds are presented in Table 2.

In some embodiments, the gRNA variant comprises one or more modifications compared to a reference guide nucleic acid scaffold sequence or a gRNA variant scaffold sequence, wherein the one or more modification is selected from: at least one nucleotide substitution in a region of the gRNA, at least one nucleotide deletion in a region of the gRNA; at least one nucleotide insertion in a region of the gRNA; a substitution of all or a portion of a region of the gRNA; a deletion of all or a portion of a region of the gRNA; or any combination of the foregoing. In some cases, the modification is a substitution of 1 to 15 consecutive or non-consecutive nucleotides in the gRNA in one or more regions. In other cases, the modification is a deletion of 1 to 10 consecutive or non-consecutive nucleotides in the gRNA in one or more regions. In other cases, the modification is an insertion of 1 to 10 consecutive or non-consecutive nucleotides in the gRNA in one or more regions. In other cases, the modification is a substitution of the scaffold stem loop or the extended stem loop with an RNA stem loop sequence from a heterologous RNA source with proximal 5′ and 3′ ends. In some cases, a gRNA variant of the disclosure comprises two or more modifications in one region relative to a gRNA. In other cases, a gRNA variant of the disclosure comprises modifications in two or more regions. In other cases, a gRNA variant comprises any combination of the foregoing modifications described in this paragraph. In some embodiments, exemplary modifications of gRNA of the disclosure include the modifications of Table 2.

In some embodiments, a 5′ G is added to a gRNA variant sequence, relative to a reference gRNA, for expression in vivo, as transcription from a U6 promoter is more efficient and more consistent with regard to the start site when the +1 nucleotide is a G. In other embodiments, two 5′ Gs are added to generate a gRNA variant sequence for in vitro transcription to increase production efficiency, as T7 polymerase strongly prefers a G in the +1 position and a purine in the +2 position. In some cases, the 5′ G bases are added to the reference scaffolds of Table 1. In other cases, the 5′ G bases are added to the variant scaffolds of Table 2.

Table 2 provides exemplary gRNA variant scaffold sequences. In some embodiments, the gRNA variant scaffold comprises any one of the sequences SEQ ID NOS: 2101-2285, 39981-40026, 40913-40958, or 41817 as listed in Table 2, or a sequence having at least about 50%, at least about 60%, at least about 70%, at least about 80%, at least about 90%, at least about 95%, at least about 95%, at least about 96%, at least about 97%, at least about 98%, at least about 99% sequence identity thereto. In some embodiments, the gRNA variant scaffold comprises any one of the sequences SEQ ID NOS: 2238-2285, 39981-40026, 40913-40958, or 41817, or a sequence having at least about 50%, at least about 60, at least about 70%, at least about 80%, at least about 90%, at least about 95%, at least about 95%, at least about 96%, at least about 97%, at least about 98%, at least about 99% sequence identity thereto. In some embodiments, the gRNA variant scaffold comprises any one of the sequences SEQ ID NOS: 2281-2285, 39981-40026, 40913-40958, or 41817, or a sequence having at least about 50%, at least about 60%, at least about 70%, at least about 80%, at least about 90%, at least about 95%, at least about 95%, at least about 96%, at least about 97%, at least about 98%, at least about 99% sequence identity thereto. It will be understood that in those embodiments wherein a vector comprises a DNA encoding sequence for a gRNA, or where a gRNA is a chimera of RNA and DNA, that thymine (T) bases can be substituted for the uracil (U) bases of any of the gRNA sequence embodiments described herein.

TABLE 2
Exemplary gRNA Scaffold Sequences

| SEQ ID | | |
|---|---|---|
| NO: | Name | NUCLEOTIDE SEQUENCE OR DESCRIPTION OF MODIFICATION |
| 2101 | ND | phage replication stable |
| 2102 | ND | Kissing loop_b1 |
| 2103 | ND | Kissing loop_a |
| 2104 | ND | 32: uvsX hairpin |
| 2105 | ND | PP7 |
| 2106 | ND | 64: trip mut, extended stem truncation |
| 2107 | ND | hyperstable tetraloop |
| 2108 | ND | C18G |
| 2109 | ND | U17G |
| 2110 | ND | CUUCGG loop |
| 2111 | ND | MS2 |
| 2112 | ND | −1, A2G, −78, G77U |
| 2113 | ND | QB |
| 2114 | ND | 45,44 hairpin |
| 2115 | ND | U1A |
| 2116 | ND | A14C, U17G |
| 2117 | ND | CUUCGG loop modified |
| 2118 | ND | Kissing loop_b2 |
| 2119 | ND | −76:78, −83:87 |
| 2120 | ND | −4 |
| 2121 | ND | extended stem truncation |
| 2122 | ND | C55 |
| 2123 | ND | trip mut |
| 2124 | ND | −76:78 |
| 2125 | ND | −1:5 |
| 2126 | ND | 83:87 |
| 2127 | ND | = +G28, A82U, −84, |
| 2128 | ND | = +51U |
| 2129 | ND | −1:4, +G5A, +G86, |
| 2130 | ND | = +A94 |
| 2131 | ND | = +G72 |
| 2132 | ND | shorten front, CUUCGG loop modified. extend extended |
| 2133 | ND | A14C |
| 2134 | ND | −1:3, +G3 |
| 2135 | ND | = +C45, +U46 |
| 2136 | ND | CUUCGG loop modified, fun start |
| 2137 | ND | −93:94 |
| 2138 | ND | = +U45 |
| 2139 | ND | −69, −94 |
| 2140 | ND | −94 |
| 2141 | ND | modified CUUCGG, minus U in 1st triplex |
| 2142 | ND | −1:4, +C4, A14C, U17G, +G72, −76:78, −83:87 |
| 2143 | ND | U1C, −73 |
| 2144 | ND | Scaffold uuCG, stem uuCG. Stem swap, t shorten |
| 2145 | ND | Scaffold uuCG, stem uuCG. Stem swap |
| 2146 | ND | = +G60 |
| 2147 | ND | no stem Scaffold uuCG |
| 2148 | ND | no stem Scaffold uuCG, fun start |
| 2149 | ND | Scaffold uuCG, stem uuCG, fun start |
| 2150 | ND | Pseudoknots |
| 2151 | ND | Scaffold uuCG, stem uuCG |
| 2152 | ND | Scaffold uuCG, stem uuCG, no start |
| 2153 | ND | Scaffold uuCG |
| 2154 | ND | = +GCUC36 |
| 2155 | ND | G quadriplex telomere basket+ ends |
| 2156 | ND | G quadriplex M3q |
| 2157 | ND | G quadriplex telomere basket no ends |
| 2158 | ND | 45, 44 hairpin (old version) |
| 2159 | ND | Sarcin-ricin loop |
| 2160 | ND | uvsX, C18G |
| 2161 | ND | truncated stem loop, C18G, trip mut (U10C) |
| 2162 | ND | short phage rep, C18G |
| 2163 | ND | phage rep loop, C18G |
| 2164 | ND | = +G18, stacked onto 64 |
| 2165 | ND | truncated stem loop, C18G, −1 A2G |
| 2166 | ND | phage rep loop, C18G, trip mut (U10C) |
| 2167 | ND | short phage rep, C18G, trip mut (U10C) |
| 2168 | ND | uvsX, trip mut (U10C) |
| 2169 | ND | truncated stem loop |
| 2170 | ND | = +A17, stacked onto 64 |
| 2171 | ND | 3′ HDV genomic ribozyme |
| 2172 | ND | phage rep loop, trip mut (U10C) |
| 2173 | ND | −79:80 |
| 2174 | ND | short phage rep, trip mut (U10C) |
| 2175 | ND | extra truncated stem loop |
| 2176 | ND | U17G, C18G |
| 2177 | ND | short phage rep |
| 2178 | ND | uvsX, C18G, −1 A2G |
| 2179 | ND | uvsX, C18G, trip mut (U10C), −1 A2G, HDV −99 G65U |
| 2180 | ND | 3′ HDV antigenomic ribozyme |
| 2181 | ND | uvsX, C18G, trip mut (U10C), −1 A2G, HDV AA(98:99)C |
| 2182 | ND | 3′ HDV ribozyme (Lior Nissim, Timothy Lu) |
| 2183 | ND | TAC(1:3)GA, stacked onto 64 |
| 2184 | ND | uvsX, −1 A2G |
| 2185 | ND | truncated stem loop, C18G, trip mut (U10C), −1 A2G, HDV −99 G65U |
| 2186 | ND | short phage rep, C18G, trip mut (U10C), −1 A2G, HDV −99 G65U |
| 2187 | ND | 3′ sTRSV WT viral Hammerhead ribozyme |
| 2188 | ND | short phage rep, C18G, −1 A2G |
| 2189 | ND | short phage rep, C18G, trip mut (U10C), −1 A2G, 3′ genomic HDV |
| 2190 | ND | phage rep loop, C18G, trip mut (U10C), −1 A2G, HDV −99 G65U |
| 2191 | ND | 3′ HDV ribozyme (Owen Ryan, Jamie Cate) |
| 2192 | ND | phage rep loop, C18G, −1 A2G |
| 2193 | ND | 0.14 |
| 2194 | ND | −78, G77U |
| 2195 | ND | ND |
| 2196 | ND | short phage rep, −1 A2G |
| 2197 | ND | truncated stem loop, C18G, trip mut (U10C), −1 A2G |
| 2198 | ND | −1, A2G |
| 2199 | ND | truncated stem loop, trip mut (U10C), −1 A2G |
| 2200 | ND | uvsX, C18G, trip mut (U10C), −1 A2G |
| 2201 | ND | phage rep loop, −1 A2G |
| 2202 | ND | phage rep loop, trip mut (U10C), −1 A2G |
| 2203 | ND | phage rep loop, C18G, trip mut (U10C), −1 A2G |
| 2204 | ND | truncated stem loop, C18G |
| 2205 | ND | uvsX, trip mut (U10C), −1 A2G |
| 2206 | ND | truncated stem loop, −1 A2G |
| 2207 | ND | short phage rep, trip mut (U10C), −1 A2G |
| 2208 | ND | 5′HDV ribozyme (Owen Ryan, Jamie Cate) |
| 2209 | ND | 5′HDV genomic ribozyme |
| 2210 | ND | truncated stem loop, C18G, trip mut (U10C), −1 A2G, HDV AA(98:99)C |
| 2211 | ND | 5′env25 pistol ribozyme (with an added CUUCGG loop) |
| 2212 | ND | 5′HDV antigenomic ribozyme |
| 2213 | ND | 3′ Hammerhead ribozyme (Lior Nissim, Timothy Lu) guide scaffold scar |
| 2214 | ND | = +A27, stacked onto 64 |
| 2215 | ND | 5′Hammerhead ribozyme (Lior Nissim, Timothy Lu) smaller scar |
| 2216 | ND | phage rep loop, C18G, trip mut (U10C), −1 A2G, HDV AA(98:99)C |
| 2217 | ND | −27, stacked onto 64 |
| 2218 | ND | 3′ Hatchet |
| 2219 | ND | 3′ Hammerhead ribozyme (Lior Nissim, Timothy Lu) |
| 2220 | ND | 5′ Hatchet |
| 2221 | ND | 5′ HDV ribozyme (Lior Nissim, Timothy Lu) |
| 2222 | ND | 5′ Hammerhead ribozyme (Lior Nissim, Timothy Lu) |
| 2223 | ND | 3′ HH15 Minimal Hammerhead ribozyme |
| 2224 | ND | 5′ RBMX recruiting motif |
| 2225 | ND | 3′ Hammerhead ribozyme (Lior Nissim, Timothy Lu) smaller scar |
| 2226 | ND | 3′ env25 pistol ribozyme (with an added CUUCGG loop) |
| 2227 | ND | 3′ Env-9 Twister |
| 2228 | ND | = +AUUAUCUCAUUACU25 |
| 2229 | ND | 5′ Env-9 Twister |
| 2230 | ND | 3′ Twisted Sister 1 |
| 2231 | ND | no stem |
| 2232 | ND | 5′ HH15 Minimal Hammerhead ribozyme |
| 2233 | ND | 5′ Hammerhead ribozyme (Lior Nissim, Timothy Lu) guide scaffold scar |
| 2234 | ND | 5′ Twisted Sister 1 |
| 2235 | ND | 5′ sTRSV WT viral Hammerhead ribozyme |
| 2236 | ND | 148: = +G55, stacked onto 64 |
| 2237 | ND | 158: 103+148(+G55) −99, G65U |
| 2238 | 174 | ACUGGCGCUUUUAUCUGAUUACUUUGAGAGCCAUCACCAGCGACUAUGUCGUAG |
| | | UGGGUAAAGCUCCCUCUUCGGAGGGAGCAUCAAAG |
| 2239 | 175 | ACUGGCGCCUUUAUCUCAUUACUUUGAGAGCCAUCACCAGCGACUAUGUCGUAU |
| | | GGGUAAAGCGCUUACGGACUUCGGUCCGUAAGAAGCAUCAAAG |
| 2240 | 176 | GCUGGCGCUUUUAUCUGAUUACUUUGAGAGCCAUCACCAGCGACUAUGUCGUAG |
| | | UGGGUAAAGCUCCCUCUUCGGAGGGAGCAUCAAAG |
| 2241 | 177 | ACUGGCGCUUUUAUCUGAUUACUUUGAGAGCCAUCACCAGCGACUAUGUCGUAU |
| | | GGGUAAAGCUCCCUCUUCGGAGGGAGCAUCAAAG |
| 2242 | 181 | ACUGGCGCCUUUAUCUGAUUACUUUGAGAGCCAUCACCAGCGACUAUGUCGUAU |
| | | GGGUAAAGCGCUUACGGACUUCGGUCCGUAAGAAGCAUCAAAG |
| 2243 | 182 | ACUGGCGCUUUUAUCUGAUUACUUUGAGAGCCAUCACCAGCGACUAUGUCGUAU |
| | | GGGUAAAGCGCUUACGGACUUCGGUCCGUAAGAAGCAUCAAAG |
| 2244 | 183 | ACUGGCGCUUUUAUCUGAUUACUUUGAGAGCCAUCACCAGCGACUAUGUCGUAG |
| | | UGGGUAAAGCGCUUACGGACUUCGGUCCGUAAGAAGCAUCAAAG |
| 2245 | 184 | ACUGGCGCUUUUAUCUGAUUACUUUGAGAGCCAUCACCAGCGACUAUGUCGUAU |
| | | UGGGUAAAGCUCCCUCUUCGGAGGGAGCAUCAAAG |
| 2246 | 185 | ACUGGCGCUUUUAUCUGAUUACUUUGAGAGCCAUCACCAGCGACUAUGUCGUAU |
| | | UGGGUAAAGCGCUUACGGACUUCGGUCCGUAAGAAGCAUCAAAG |
| 2247 | 186 | ACUGGCGCCUUUAUCAUCAUUACUUUGAGAGCCAUCACCAGCGACUAUGUCGUA |
| | | UGGGUAAAGCGCUUACGGACUUCGGUCCGUAAGAAGCAUCAAAG |
| 2248 | 187 | ACUGGCGCUUUUAUCUGAUUACUUUGAGAGCCAUCACCAGCGACUAUGUCGUAG |
| | | UGGGUAAAGCGCCCUCUUCGGAGGGAAGCAUCAAAG |
| 2249 | 188 | ACUGGCGCUUUUAUCUGAUUACUUUGAGAGCCAUCACCAGCGACUAUGUCGUAG |
| | | UGGGUAAAGCUCACAUGAGGAUCACCCAUGUGAGCAUCAAAG |
| 2250 | 189 | ACUGGCACUUUUACCUGAUUACUUUGAGAGCCAACACCAGCGACUAUGUCGUAG |
| | | UGGGUAAAGCUCCCUCUUCGGAGGGAGCAUCAAAG |
| 2251 | 190 | ACUGGCACUUUUAUCUGAUUACUUUGAGAGCCAUCACCAGCGACUAUGUCGUAG |
| | | UGGGUAAAGCUCCCUCUUCGGAGGGAGCAUCAAAG |
| 2252 | 191 | ACUGGCCCUUUUAUCUGAUUACUUUGAGAGCCAUCACCAGCGACUAUGUCGUAG |
| | | UGGGUAAAGCUCCCUCUUCGGAGGGAGCAUCAAAG |
| 2253 | 192 | ACUGGCGCUUUUACCUGAUUACUUUGAGAGCCAUCACCAGCGACUAUGUCGUAG |
| | | UGGGUAAAGCUCCCUCUUCGGAGGGAGCAUCAAAG |
| 2254 | 193 | ACUGGCGCUUUUAUCUGAUUACUUUGAGAGCCAACACCAGCGACUAUGUCGUAG |
| | | UGGGUAAAGCUCCCUCUUCGGAGGGAGCAUCAAAG |
| 2255 | 195 | ACUGGCACCUUUACCUGAUUACUUUGAGAGCCAACACCAGCGACUAUGUCGUAU |
| | | GGGUAAAGCGCUUACGGACUUCGGUCCGUAAGAAGCAUCAAAG |
| 2256 | 196 | ACUGGCACCUUUAUCUGAUUACUUUGAGAGCCAUCACCAGCGACUAUGUCGUAU |
| | | GGGUAAAGCGCUUACGGACUUCGGUCCGUAAGAAGCAUCAAAG |
| 2257 | 197 | ACUGGCCCCUUUAUCUGAUUACUUUGAGAGCCAUCACCAGCGACUAUGUCGUAU |
| | | GGGUAAAGCGCUUACGGACUUCGGUCCGUAAGAAGCAUCAAAG |
| 2258 | 198 | ACUGGCGCCUUUAUCUGAUUACUUUGAGAGCCAACACCAGCGACUAUGUCGUAU |
| | | GGGUAAAGCGCUUACGGACUUCGGUCCGUAAGAAGCAUCAAAG |
| 2259 | 199 | GCUGGCGCUUUUAUCUGAUUACUUUGAGAGCCAUCACCAGCGACUAUGUCGUAG |
| | | UGGGUAAAGCUCCCUCUUCGGAGGGAGCAUCAAAG |
| 2260 | 200 | GACUGGCGCUUUUAUCUGAUUACUUUGAGAGCCAUCACCAGCGACUAUGUCGUA |
| | | GUGGGUAAAGCUCCCUCUUCGGAGGGAGCAUCAAAG |
| 2261 | 201 | ACUGGCGCCUUUAUCUGAUUACUUUGGAGAGCCAUCACCAGCGACUAUGUCGUA |
| | | GUGGGUAAAGCUCCCUCUUCGGAGGGAGCAUCAAAG |
| 2262 | 202 | ACUGGCGCAUUUAUCUGAUUACUUUGUGAGCCAUCACCAGCGACUAUGUCGUAG |
| | | UGGGUAAAGCUCCCUCUUCGGAGGGAGCAUCAAAG |
| 2263 | 203 | ACUGGCGCCUUUAUCUGAUUACUUUGAGAGCCAUCACCAGCGACUAUGUCGUAG |
| | | UGGGUAAAGCUCCCUCUUCGGAGGGAGCAUCAAAG |
| 2264 | 204 | ACUGGCGCUUUUAUCUGAUUACUUUGGAGAGCCAUCACCAGCGACUAUGUCGUA |
| | | GUGGGUAAAGCUCCCUCUUCGGAGGGAGCAUCAAAG |
| 2265 | 205 | ACUGGCGCAUUUAUCUGAUUACUUUGAGAGCCAUCACCAGCGACUAUGUCGUAG |
| | | UGGGUAAAGCUCCCUCUUCGGAGGGAGCAUCAAAG |
| 2266 | 206 | ACUGGCGCUUUUAUCUGAUUACUUUGUGAGCCAUCACCAGCGACUAUGUCGUAG |
| | | UGGGUAAAGCUCCCUCUUCGGAGGGAGCAUCAAAG |
| 2267 | 207 | ACUGGCGCUUUUAUUCUGAUUACUUUGAGAGCCAUCACCAGCGACUAUGUCGUA |
| | | GUGGGUAAAGCUCCCUCUUCGGAGGGAGCAUCAAAG |
| 2268 | 208 | ACGGCGCUUUUAUCUGAUUACUUUGAGAGCCAUCACCAGCGACUAUGUCGUAGU |
| | | GGGUAAAGCUCCCUCUUCGGAGGGAGCAUCAAAG |
| 2269 | 209 | ACUGGCGCUUUUAUAUGAUUACUUUGAGAGCCAUCACCAGCGACUAUGUCGUAG |
| | | UGGGUAAAGCUCCCUCUUCGGAGGGAGCAUCAAAG |
| 2270 | 210 | ACUGGCGCUUUUAUCUUGAUUACUUUGAGAGCCAUCACCAGCGACUAUGUCGUA |
| | | GUGGGUAAAGCUCCCUCUUCGGAGGGAGCAUCAAAG |
| 2271 | 211 | ACUGGCGCUUUUAUCUGAUUACUUUGAGAGCCAGCACCAGCGACUAUGUCGUAG |
| | | UGGGUAAAGCUCCCUCUUCGGAGGGAGCAUCAAAG |
| 2272 | 212 | ACUGGCGCUGUUAUCUGAUUACUUCGAGAGCCAUCACCAGCGACUAUGUCGUAG |
| | | UGGGUAAAGCUCCCUCUUCGGAGGGAGCAUCGAAG |
| 2273 | 213 | ACUGGCGCUCUUAUCUGAUUACUUCGAGAGCCAUCACCAGCGACUAUGUCGUAG |
| | | UGGGUAAAGCUCCCUCUUCGGAGGGAGCAUCGAAG |
| 2274 | 214 | ACUGGCGCUUGUAUCUGAUUACUCUGAGAGCCAUCACCAGCGACUAUGUCGUAG |
| | | UGGGUAAAGCUCCCUCUUCGGAGGGAGCAUCAGAG |
| 2275 | 215 | ACUGGCGCUUCUAUCUGAUUACUCUGAGAGCCAUCACCAGCGACUAUGUCGUAG |
| | | UGGGUAAAGCUCCCUCUUCGGAGGGAGCAUCAGAG |
| 2276 | 216 | ACUGGCGCUUUGAUCUGAUUACCUUGAGAGCCAUCACCAGCGACUAUGUCGUAG |
| | | UGGGUAAAGCUCCCUCUUCGGAGGGAGCAUCAAGG |
| 2277 | 217 | ACUGGCGCUUUCAUCUGAUUACCUUGAGAGCCAUCACCAGCGACUAUGUCGUAG |
| | | UGGGUAAAGCUCCCUCUUCGGAGGGAGCAUCAAGG |
| 2278 | 218 | ACUGGCGCUGUUAUCUGAUUACUUUGAGAGCCAUCACCAGCGACUAUGUCGUAG |
| | | UGGGUAAAGCUCCCUCUUCGGAGGGAGCAUCAAAG |
| 2279 | 219 | ACUGGCGCUUUUAUCUGAUUACUUUGAGAGCCAUCACCAGCGACUAUGUCGUAG |
| | | UGGGUAAAGCUCCCUCUUCGGAGGGAGCAUCGAAG |
| 2280 | 220 | ACUGGCGCUUUUAUCUGAUUACUUCGAGAGCCAUCACCAGCGACUAUGUCGUAG |
| | | UGGGUAAAGCUCCCUCUUCGGAGGGAGCAUCAAAG |
| 2281 | 221 | ACUGGCACUUCUAUCUGAUUACUCUGAGAGCCAUCACCAGCGACUAUGUCGUAU |
| | | GGGUAAAGCCGCUUACGGACUUCGGUCCGUAAGAGGCAUCAGAG |
| 2282 | 222 | ACUGGCACUUCUAUCUGAUUACUCUGAGAGCCAUCACCAGCGACUAUGUCGUAG |
| | | UGGGUAAAGCUCCCUCUUCGGAGGGAGCAUCAGAG |
| 2283 | 223 | ACUGGCACCUUUAUCUGAUUACUUUGAGAGCCAUCACCAGCGACUAUGUCGUAU |
| | | GGGUAAAGCCGCUUACGGACUUCGGUCCGUAAGAGGCAUCAAAG |
| 2284 | 224 | ACUGGCACUUGUAUCUGAUUACUCUGAGAGCCAUCACCAGCGACUAUGUCGUAU |
| | | GGGUAAAGCCGCUUACGGACUUCGGUCCGUAAGAGGCAUCAGAG |
| 2285 | 225 | ACUGGCACUUGUAUCUGAUUACUCUGAGAGCCAUCACCAGCGACUAUGUCGUAG |
| | | UGGGUAAAGCUCCCUCUUCGGAGGGAGCAUCAGAG |
| 41817 | 226 | ACUGGCGCUUUUAUCUGAUUACUUUGAGAGCCAUCACCAGCGACUAUGUCGUAG |
| | | UGGGUAAAGCUGCACUAUGGGCGCAGCGUCAAUGACGCUGACGGUACAGGCCAG |
| | | ACAAUUAUUGUCUGGUAUAGUGCAGCAUCAAAG |
| 39981 | 229 | ACUGGCGCUUUUAUCUGAUUACUUUGAGAGCCAUCACCAGCGACUAUGUCGUAG |
| | | UGGGUAAAGCUCCCCGUACACCAUUAGGGUACGGGGAGCAUCAAAGCGAGACGU |
| | | AAUUACGUCUCGUUUUUUUU |
| 39982 | 230 | ACUGGCACUUCUAUCUGAUUACUCUGAGAGCCAUCACCAGCGACUAUGUCGUAU |
| | | GGGUAAAGCGCUUACGGACUUCGGUCCGUAAGAAGCAUCAGAG |
| 39983 | 231 | ACUGGCGCUUCUAUCUGAUUACUCUGAGAGCCAUCACCAGCGACUAUGUCGUAU |
| | | GGGUAAAGCCGCUUACGGACUUCGGUCCGUAAGAGGCAUCAGAG |
| 39984 | 232 | ACUGGCACUUCUAUCUGAUUACUCUGAGCGCCAUCACCAGCGACUAUGUCGUAU |
| | | GGGUAAAGCCGCUUACGGACUUCGGUCCGUAAGAGGCAUCAGAG |
| 39985 | 233 | ACUGGCGCUUCUAUCUGAUUACUCUGAGCGCCAUCACCAGCGACUAUGUCGUAU |
| | | GGGUAAAGCCGCUUACGGACUUCGGUCCGUAAGAGGCAUCAGAG |
| 39986 | 234 | ACUGGCGCUUCUAUCUGAUUACUCUGAGCGCCAUCACCAGCGACUAUGUCGUAU |
| | | GGGUAAAGCGCCUUACGGACUUCGGUCCGUAAGGAGCAUCAGAG |
| 39987 | 235 | ACUGGCGCUUCUAUCUGAUUACUCUGAGCGCCAUCACCAGCGACUAUGUCGUAG |
| | | UGGGUAAAGCCGCUUACGGACUUCGGUCCGUAAGAGGCAUCAGAG |
| 39988 | 236 | ACGGGACUUUCUAUCUGAUUACUCUGAAGUCCCUCACCAGCGACUAUGUCGUAU |
| | | GGGUAAAGCCGCUUACGGACUUCGGUCCGUAAGAGGCAUCAGAG |
| 39989 | 237 | ACCUGUAGUUCUAUCUGAUUACUCUGACUACAGUCACCAGCGACUAUGUCGUAU |
| | | GGGUAAAGCCGCUUACGGACUUCGGUCCGUAAGAGGCAUCAGAG |
| 39990 | 238 | ACUGGCGCUUUUAUCUGAUUACUUUGAGAGCCAUCACCAGCGACUAUGUCGUAG |
| | | UGGGUAAAGCUGCACGGUGGGCGCAGCUUCGGCUGACGGUACACCGUGCAGCAU |
| | | CAAAG |
| 39991 | 239 | ACUGGCGCUUUUAUCUGAUUACUUUGAGAGCCAUCACCAGCGACUAUGUCGUAG |
| | | UGGGUAAAGCUGCACGGUGGGCGCAGCUUCGGCUGACGGUACACCGGUGGGCGC |
| | | AGCUUCGGCUGACGGUACACCGUGCAGCAUCAAAG |
| 39992 | 240 | ACUGGCGCUUUUAUCUGAUUACUUUGAGAGCCAUCACCAGCGACUAUGUCGUAG |
| | | UGGGUAAAGCUGCACGGUGGGCGCAGCUUCGGCUGACGGUACACCGGUGGGCGC |
| | | AGCUUCGGCUGACGGUACACCGGUGGGCGCAGCUUCGGCUGACGGUACACCGUG |
| | | CAGCAUCAAAG |
| 39993 | 241 | ACUGGCGCUUUUAUCUGAUUACUUUGAGAGCCAUCACCAGCGACUAUGUCGUAG |
| | | UGGGUAAAGCUGCACGGUGGGCGCAGCUUCGGCUGACGGUACACCGGUGGGCGC |
| | | AGCUUCGGCUGACGGUACACCGGUGGGCGCAGCUUCGGCUGACGGUACACCGGU |
| | | GGGCGCAGCUUCGGCUGACGGUACACCGUGCAGCAUCAAAG |
| 39994 | 242 | ACUGGCGCUUUUAUCUGAUUACUUUGAGAGCCAUCACCAGCGACUAUGUCGUAG |
| | | UGGGUAAAGCUGCACGGUGGGCGCAGCUUCGGCUGACGGUACACCGGUGGGCGC |
| | | AGCUUCGGCUGACGGUACACCGGUGGGCGCAGCUUCGGCUGACGGUACACCGGU |
| | | GGGCGCAGCUUCGGCUGACGGUACACCGGUGGGCGCAGCUUCGGCUGACGGUAC |
| | | ACCGUGCAGCAUCAAAG |
| 39995 | 243 | ACUGGCGCUUUUAUCUGAUUACUUUGAGAGCCAUCACCAGCGACUAUGUCGUAG |
| | | UGGGUAAAGCUGCACCUAGCGGAGGCUAGGUGCAGCAUCAAAG |
| 39996 | 244 | ACUGGCGCUUUUAUCUGAUUACUUUGAGAGCCAUCACCAGCGACUAUGUCGUAG |
| | | UGGGUAAAGCUGCACCUCGGCUUGCUGAAGCGCGCACGGCAAGAGGCGAGGUGC |
| | | AGCAUCAAAG |
| 39997 | 245 | ACUGGCGCUUUUAUCUGAUUACUUUGAGAGCCAUCACCAGCGACUAUGUCGUAG |
| | | UGGGUAAAGCUGCACCUCUCUCGACGCAGGACUCGGCUUGCUGAAGCGCGCACG |
| | | GCAAGAGGCGAGGGGCGGCGACUGGUGAGUACGCCAAAAAUUUUGACUAGCGGA |
| | | GGCUAGAAGGAGAGAGGUGCAGCAUCAAAG |
| 39998 | 246 | ACUGGCGCUUUUAUCUGAUUACUUUGAGAGCCAUCACCAGCGACUAUGUCGUAG |
| | | UGGGUAAAGCUGCACGGUGCCCGUCUGUUGUGUCGAGAGACGCCAAAAAUUUUG |
| | | ACUAGCGGAGGCUAGAAGGAGAGAGAUGGGUGCCGUGCAGCAUCAAAG |
| 39999 | 247 | ACUGGCGCUUUUAUCUGAUUACUUUGAGAGCCAUCACCAGCGACUAUGUCGUAG |
| | | UGGGUAAAGCUGCACAUGGAGAGGAGAUGUGCAGCAUCAAAG |
| 40000 | 248 | ACUGGCGCUUUUAUCUGAUUACUUUGAGAGCCAUCACCAGCGACUAUGUCGUAG |
| | | UGGGUAAAGCUGCACAUGGAGAUGUGCAGCAUCAAAG |
| 40001 | 249 | ACUGGCGCUUUUAUCUGAUUACUUUGAGAGCCAUCACCAGCGACUAUGUCGUAG |
| | | UGGGUAAAGCUUGGGCGCAGCGUCAAUGACGCUGACGGUACAAGCAUCAAAG |
| 40002 | 250 | ACUGGCGCUUUUAUCUGAUUACUUUGAGAGCCAUCACCAGCGACUAUGUCGUAG |
| | | UGGGUAAAGCUGCACUAUGGGCGCAGCGUCAAUGACGCUGACGGUACAGGCCAC |
| | | AUGAGGAUCACCCAUGUGGUAUAGUGCAGCAUCAAAG |
| 40003 | 251 | ACUGGCGCUUUUAUCUGAUUACUUUGAGAGCCAUCACCAGCGACUAUGUCGUAG |
| | | UGGGUAAAGCUGCACUAUGGGCGCAGCUCAUGAGGAUCACCCAUGAGCUGACGG |
| | | UACAGGCCACAUGAGGAUCACCCAUGUGGUAUAGUGCAGCAUCAAAG |
| 40004 | 252 | ACUGGCGCUUUUAUCUGAUUACUUUGAGAGCCAUCACCAGCGACUAUGUCGUAG |
| | | UGGGUAAAGCUGCACUAUGGGCGCAGCGUCAAUGACGCUGACGGUACAGGCCAC |
| | | AUGGCAGUCGUAACGACGCGGGUGGUAUAGUGCAGCAUCAAAG |
| 40005 | 253 | ACUGGCGCUUUUAUCUGAUUACUUUGAGAGCCAUCACCAGCGACUAUGUCGUAG |
| | | UGGGUAAAGCUGCACUAUGGGCGCAGCAAACAUGGCAGUCCUAAGGACGCGGGU |
| | | UUUGCUGACGGUACAGGCCACAUGGCAGUCGUAACGACGCGGGUGGUAUAGUGC |
| | | AGCAUCAAAG |
| 40006 | 254 | ACUGGCGCUUUUAUCUGAUUACUUUGAGAGCCAUCACCAGCGACUAUGUCGUAG |
| | | UGGGUAAAGCUGCACUAUGGGCGCAGACAUGGCAGUCGUAACGACGCGGGUCUG |
| | | ACGGUACAGGCCACAUGAGGAUCACCCAUGUGGUAUAGUGCAGCAUCAAAG |
| 40007 | 255 | ACUGGCGCUUUUAUCUGAUUACUUUGAGAGCCAUCACCAGCGACUAUGUCGUAG |
| | | UGGGUAAAGCUGCACUAAGGAGUUUAUAUGGAAACCCUUAGUGCAGCAUCAAAG |
| 40008 | 256 | ACUGGCGCUUUUAUCUGAUUACUUUGAGAGCCAUCACCAGCGACUAUGUCGUAG |
| | | UGGGUAAAGCUCAGGAAGCACUAUGGGCGCAGCGUCAAUGACGCUGACGGUACA |
| | | GGCCAGACAAUUAUUGUCUGGUAUAGUGCAGCAGCAGAACAAUUUGCUGAGGGC |
| | | UAUUGAGGCGCAACAGCAUCUGUUGCAACUCACAGUCUGGGGCAUCAAGCAGCU |
| | | CCAGGCAAGAAUCCUGAGCAUCAAAG |
| 40009 | 257 | ACUGGCGCUUUUAUCUGAUUACUUUGAGAGCCAUCACCAGCGACUAUGUCGUAG |
| | | UGGGUAAAGCUGCACGCCCUGAAGAAGGGCGUGCAGCAUCAAAG |
| 40010 | 258 | ACUGGCGCUUUUAUCUGAUUACUUUGAGAGCCAUCACCAGCGACUAUGUCGUAG |
| | | UGGGUAAAGCUGCACGGCUCGUGUAGCUCAUUAGCUCCGAGCCGUGCAGCAUCA |
| | | AAG |
| 40011 | 259 | ACUGGCGCUUUUAUCUGAUUACUUUGAGAGCCAUCACCAGCGACUAUGUCGUAG |
| | | UGGGUAAAGCUGCACCCGUGUGCAUCCGCAGUGUCGGAUCCACGGGUGCAGCAU |
| | | CAAAG |
| 40012 | 260 | ACUGGCGCUUUUAUCUGAUUACUUUGAGAGCCAUCACCAGCGACUAUGUCGUAG |
| | | UGGGUAAAGCUGCACGGAAUCCAUUGCACUCCGGAUUUCACUAGGUGCAGCAUC |
| | | AAAG |
| 40013 | 261 | ACUGGCGCUUUUAUCUGAUUACUUUGAGAGCCAUCACCAGCGACUAUGUCGUAG |
| | | UGGGUAAAGCUGCACAUGCAUGUCUAAGACAGCAUGUGCAGCAUCAAAG |
| 40014 | 262 | ACUGGCGCUUUUAUCUGAUUACUUUGAGAGCCAUCACCAGCGACUAUGUCGUAG |
| | | UGGGUAAAGCUGCACAAAACAUAAGGAAAACCUAUGUUGUGCAGCAUCAAAG |
| 40015 | 263 | ACUGGCGCUUCUAUCUGAUUACUCUGAGCGCCAUCACCAGCGACUAUGUCGUAG |
| | | UGGGUAAAGCCGCUUACGGACUAUGGGCGCAGCGUCAAUGACGCUGACGGUACA |
| | | GGCCAGACAAUUAUUGUCUGGUAUAGUCCGUAAGAGGCAUCAGAG |
| 40016 | 264 | ACUGGCGCUUCUAUCUGAUUACUCUGAGCGCCAUCACCAGCGACUAUGUCGUAG |
| | | UGGGUAAAGCCGCUUACGGGUGGGCGCAGCGUCAAUGACGCUGACGGUACAGGC |
| | | CAGACAAUUAUUGUCUGGUACCCGUAAGAGGCAUCAGAG |
| 40017 | 265 | ACUGGCGCUUCUAUCUGAUUACUCUGAGCGCCAUCACCAGCGACUAUGUCGUAG |
| | | UGGGUAAAGCCGCUUACGGUAUGGGCGCAGCGUCAAUGACGCUGACGGUACAGG |
| | | CCACAUGAGGAUCACCCAUGUGGUAUACCGUAAGAGGCAUCAGAG |
| 40018 | 266 | ACUGGCGCUUUUAUCUGAUUACUUUGAGAGCCAUCACCAGCGACUAUGUCGUAG |
| | | UGGGUAAAGCUCCCUAUGGGCGCAGCGUCAAUGACGCUGACGGUACAGGCCACA |
| | | UGAGGAUCACCCAUGUGGUAUAGGGAGCAUCAAAG |
| 40019 | 267 | ACUGGCGCUUCUAUCUGAUUACUCUGAGCGCCAUCACCAGCGACUAUGUCGUAG |
| | | UGGGUAAAGCCGCUUACGGUAUGGGCGCAGCUCAUGAGGAUCACCCAUGAGCUG |
| | | ACGGUACAGGCCACAUGAGGAUCACCCAUGUGGUAUACCGUAAGAGGCAUCAGA |
| | | G |
| 40020 | 268 | ACUGGCGCUUUUAUCUGAUUACUUUGAGAGCCAUCACCAGCGACUAUGUCGUAG |
| | | UGGGUAAAGCUCCCUAUGGGCGCAGCUCAUGAGGAUCACCCAUGAGCUGACGGU |
| | | ACAGGCCACAUGAGGAUCACCCAUGUGGUAUAGGGAGCAUCAAAG |
| 40021 | 269 | ACUGGCGCUUCUAUCUGAUUACUCUGAGCGCCAUCACCAGCGACUAUGUCGUAG |
| | | UGGGUAAAGCCGCUUACGGUAUGGGCGCAGCGUCAAUGACGCUGACGGUACAGG |
| | | CCACAUGGCAGUCGUAACGACGCGGGUGGUAUACCGUAAGAGGCAUCAGAG |
| 40022 | 270 | ACUGGCGCUUUUAUCUGAUUACUUUGAGAGCCAUCACCAGCGACUAUGUCGUAG |
| | | UGGGUAAAGCUCCCUAUGGGCGCAGCGUCAAUGACGCUGACGGUACAGGCCACA |
| | | UGGCAGUCGUAACGACGCGGGUGGUAUAGGGAGCAUCAAAG |
| 40023 | 271 | ACUGGCGCUUCUAUCUGAUUACUCUGAGCGCCAUCACCAGCGACUAUGUCGUAG |
| | | UGGGUAAAGCCGCUUACGGUAUGGGCGCAGCAAACAUGGCAGUCCUAAGGACGC |
| | | GGGUUUUGCUGACGGUACAGGCCACAUGGCAGUCGUAACGACGCGGGUGGUAUA |
| | | CCGUAAGAGGCAUCAGAG |
| 40024 | 272 | ACUGGCGCUUUUAUCUGAUUACUUUGAGAGCCAUCACCAGCGACUAUGUCGUAG |
| | | UGGGUAAAGCUCCCUAUGGGCGCAGCAAACAUGGCAGUCCUAAGGACGCGGGUU |
| | | UUGCUGACGGUACAGGCCACAUGGCAGUCGUAACGACGCGGGUGGUAUAGGGAG |
| | | CAUCAAAG |
| 40025 | 273 | ACUGGCGCUUCUAUCUGAUUACUCUGAGCGCCAUCACCAGCGACUAUGUCGUAG |
| | | UGGGUAAAGCCGCUUACGGUAUGGGCGCAGACAUGGCAGUCGUAACGACGCGGG |
| | | UCUGACGGUACAGGCCACAUGAGGAUCACCCAUGUGGUAUACCGUAAGAGGCAU |
| | | CAGAG |
| 40026 | 274 | ACUGGCGCUUUUAUCUGAUUACUUUGAGAGCCAUCACCAGCGACUAUGUCGUAG |
| | | UGGGUAAAGCUCCCUAUGGGCGCAGACAUGGCAGUCGUAACGACGCGGGUCUGA |
| | | CGGUACAGGCCACAUGAGGAUCACCCAUGUGGUAUAGGGAGCAUCAAAG |
| 40913 | 275 | ACUGGCGCUUUUAUCUGAUUACUUUGAGAGCCAUCACCAGCGACUAUGU |
| | | CGUAGUGGGUAAAGCUGCACUAUGGGCGCAGCACCUGAGGAUCACCCAG |
| | | GUGCUGACGGUACAGGCCACCUGAGGAUCACCCAGGUGGUAUAGUGCAG |
| | | CAUCAAAG |
| 40914 | 276 | ACUGGCGCUUUUAUCUGAUUACUUUGAGAGCCAUCACCAGCGACUAUGU |
| | | CGUAGUGGGUAAAGCUGCACUAUGGGCGCAGCGCAUGAGGAUCACCCAU |
| | | GCGCUGACGGUACAGGCCGCAUGAGGAUCACCCAUGCGGUAUAGUGCAG |
| | | CAUCAAAG |
| 40915 | 277 | ACUGGCGCUUUUAUCUGAUUACUUUGAGAGCCAUCACCAGCGACUAUGU |
| | | CGUAGUGGGUAAAGCUGCACUAUGGGCGCAGCGCCUGAGGAUCACCCAG |
| | | GCGCUGACGGUACAGGCCGCCUGAGGAUCACCCAGGCGGUAUAGUGCAG |
| | | CAUCAAAG |
| 40916 | 278 | ACUGGCGCUUUUAUCUGAUUACUUUGAGAGCCAUCACCAGCGACUAUGU |
| | | CGUAGUGGGUAAAGCUGCACUAUGGGCGCAGCGCCUGAGCAUCAGCCAG |
| | | GCGCUGACGGUACAGGCCGCCUGAGCAUCAGCCAGGCGGUAUAGUGCAG |
| | | CAUCAAAG |
| 40917 | 279 | ACUGGCGCUUUUAUCUGAUUACUUUGAGAGCCAUCACCAGCGACUAUGU |
| | | CGUAGUGGGUAAAGCUGCACUAUGGGCGCAGCACAUGAGCAUCAGCCAU |
| | | GUGCUGACGGUACAGGCCACAUGAGCAUCAGCCAUGUGGUAUAGUGCAG |
| | | CAUCAAAG |
| 40918 | 280 | ACUGGCGCUUUUAUCUGAUUACUUUGAGAGCCAUCACCAGCGACUAUGU |
| | | CGUAGUGGGUAAAGCUGCACUAUGGGCGCAGCACAUGAGUAUCAACCAU |
| | | GUGCUGACGGUACAGGCCACAUGAGUAUCAACCAUGUGGUAUAGUGCAG |
| | | CAUCAAAG |
| 40919 | 281 | ACUGGCGCUUUUAUCUGAUUACUUUGAGAGCCAUCACCAGCGACUAUGU |
| | | CGUAGUGGGUAAAGCUGCACUAUGGGCGCAGCACAUGAGAAUCAGCCAU |
| | | GUGCUGACGGUACAGGCCACAUGAGAAUCAGCCAUGUGGUAUAGUGCAG |
| | | CAUCAAAG |
| 40920 | 282 | ACUGGCGCUUUUAUCUGAUUACUUUGAGAGCCAUCACCAGCGACUAUGU |
| | | CGUAGUGGGUAAAGCUGCACUAUGGGCGCAGCCCUUGAGGAUCACCCAU |
| | | GUGCUGACGGUACAGGCCCCUUGAGGAUCACCCAUGUGGUAUAGUGCAG |
| | | CAUCAAAG |
| 40921 | 283 | ACUGGCGCUUUUAUCUGAUUACUUUGAGAGCCAUCACCAGCGACUAUGU |
| | | CGUAGUGGGUAAAGCUGCACUAUGGGCGCAGCACUUGAGGAUCACCCAU |
| | | GUGCUGACGGUACAGGCCACUUGAGGAUCACCCAUGUGGUAUAGUGCAG |
| | | CAUCAAAG |
| 40922 | 284 | ACUGGCGCUUUUAUCUGAUUACUUUGAGAGCCAUCACCAGCGACUAUGU |
| | | CGUAGUGGGUAAAGCUGCACUAUGGGCGCAGCACCUGAGGAUCACCCAU |
| | | GUGCUGACGGUACAGGCCACCUGAGGAUCACCCAUGUGGUAUAGUGCAG |
| | | CAUCAAAG |
| 40923 | 285 | ACUGGCGCUUUUAUCUGAUUACUUUGAGAGCCAUCACCAGCGACUAUGUCGUAG |
| | | UGGGUAAAGCUGCACUAUGGGCGCAGCACAUGAGGAUCACCUAUGUGCUGACGG |
| | | UACAGGCCACAUGAGGAUCACCUAUGUGGUAUAGUGCAGCAUCAAAG |
| 40924 | 286 | ACUGGCGCUUUUAUCUGAUUACUUUGAGAGCCAUCACCAGCGACUAUGUCGUAG |
| | | UGGGUAAAGCUGCACUAUGGGCGCAGCACAUUAGGAUCACCAAUGUGCUGACGG |
| | | UACAGGCCACAUUAGGAUCACCAAUGUGGUAUAGUGCAGCAUCAAAG |
| 40925 | 287 | ACUGGCGCUUUUAUCUGAUUACUUUGAGAGCCAUCACCAGCGACUAUGUCGUAG |
| | | UGGGUAAAGCUGCACUAUGGGCGCAGCACAUUAGGAUCACCGAUGUGCUGACGG |
| | | UACAGGCCACAUUAGGAUCACCGAUGUGGUAUAGUGCAGCAUCAAAG |
| 40926 | 288 | ACUGGCGCUUUUAUCUGAUUACUUUGAGAGCCAUCACCAGCGACUAUGUCGUAG |
| | | UGGGUAAAGCUGCACUAUGGGCGCAGCACAUUAGGAUCACCUAUGUGCUGACGG |
| | | UACAGGCCACAUUAGGAUCACCUAUGUGGUAUAGUGCAGCAUCAAAG |
| 40927 | 289 | ACUGGCGCUUUUAUCUGAUUACUUUGAGAGCCAUCACCAGCGACUAUGUCGUAG |
| | | UGGGUAAAGCUGCACUAUGGGCGCAGCACAUGAGGAUUACCCAUGUGCUGACGG |
| | | UACAGGCCACAUGAGGAUUACCCAUGUGGUAUAGUGCAGCAUCAAAG |
| 40928 | 290 | ACUGGCGCUUUUAUCUGAUUACUUUGAGAGCCAUCACCAGCGACUAUGUCGUAG |
| | | UGGGUAAAGCUGCACUAUGGGCGCAGCACAUGAGGAUAACCCAUGUGCUGACGG |
| | | UACAGGCCACAUGAGGAUAACCCAUGUGGUAUAGUGCAGCAUCAAAG |
| 40929 | 291 | ACUGGCGCUUUUAUCUGAUUACUUUGAGAGCCAUCACCAGCGACUAUGUCGUAG |
| | | UGGGUAAAGCUGCACUAUGGGCGCAGCACAUGAGGAUGACCCAUGUGCUGACGG |
| | | UACAGGCCACAUGAGGAUGACCCAUGUGGUAUAGUGCAGCAUCAAAG |
| 40930 | 292 | ACUGGCGCUUUUAUCUGAUUACUUUGAGAGCCAUCACCAGCGACUAUGUCGUAG |
| | | UGGGUAAAGCUGCACUAUGGGCGCAGCACAUGAGGACCACCCAUGUGCUGACGG |
| | | UACAGGCCACAUGAGGACCACCCAUGUGGUAUAGUGCAGCAUCAAAG |
| 40931 | 293 | ACUGGCGCUUUUAUCUGAUUACUUUGAGAGCCAUCACCAGCGACUAUGUCGUAG |
| | | UGGGUAAAGCUGCACUAUGGGCGCAGCAGAUGAGGAUCACCCAUGGGCUGACGG |
| | | UACAGGCCAGAUGAGGAUCACCCAUGGGGUAUAGUGCAGCAUCAAAG |
| 40932 | 294 | ACUGGCGCUUUUAUCUGAUUACUUUGAGAGCCAUCACCAGCGACUAUGUCGUAG |
| | | UGGGUAAAGCUGCACUAUGGGCGCAGCACAUGGGGAUCACCCAUGUGCUGACGG |
| | | UACAGGCCACAUGGGGAUCACCCAUGUGGUAUAGUGCAGCAUCAAAG |
| 40933 | 295 | ACUGGCGCUUUUAUCUGAUUACUUUGAGAGCCAUCACCAGCGACUAUGUCGUAG |
| | | UGGGUAAAGCUGCACUAUGGGCGCAGCACAUGAGGAUCACCCAUGUGCUGACGG |
| | | UACAGGCCACAUGAGGAUCACCCAUGUGGUAUAGUGCAGCAUCAAAG |
| 40934 | 296 | ACUGGCGCUUUUAUCUGAUUACUUUGAGAGCCAUCACCAGCGACUAUGUCGUAG |
| | | UGGGUAAAGCUCACCUGAGGAUCACCCAGGUGAGCAUCAAAG |
| 40935 | 297 | ACUGGCGCUUUUAUCUGAUUACUUUGAGAGCCAUCACCAGCGACUAUGUCGUAG |
| | | UGGGUAAAGCUCGCAUGAGGAUCACCCAUGCGAGCAUCAAAG |
| 40936 | 298 | ACUGGCGCUUUUAUCUGAUUACUUUGAGAGCCAUCACCAGCGACUAUGUCGUAG |
| | | UGGGUAAAGCUCGCCUGAGGAUCACCCAGGCGAGCAUCAAAG |
| 40937 | 299 | ACUGGCGCUUUUAUCUGAUUACUUUGAGAGCCAUCACCAGCGACUAUGUCGUAG |
| | | UGGGUAAAGCUCGCCUGAGCAUCAGCCAGGCGAGCAUCAAAG |
| 40938 | 300 | ACUGGCGCUUUUAUCUGAUUACUUUGAGAGCCAUCACCAGCGACUAUGUCGUAG |
| | | UGGGUAAAGCUCACAUGAGCAUCAGCCAUGUGAGCAUCAAAG |
| 40939 | 301 | ACUGGCGCUUUUAUCUGAUUACUUUGAGAGCCAUCACCAGCGACUAUGUCGUAG |
| | | UGGGUAAAGCUCACAUGAGUAUCAACCAUGUGAGCAUCAAAG |
| 40940 | 302 | ACUGGCGCUUUUAUCUGAUUACUUUGAGAGCCAUCACCAGCGACUAUGUCGUAG |
| | | UGGGUAAAGCUCACAUGAGAAUCAGCCAUGUGAGCAUCAAAG |
| 40941 | 303 | ACUGGCGCUUUUAUCUGAUUACUUUGAGAGCCAUCACCAGCGACUAUGUCGUAG |
| | | UGGGUAAAGCUCCCUUGAGGAUCACCCAUGUGAGCAUCAAAG |
| 40942 | 304 | ACUGGCGCUUUUAUCUGAUUACUUUGAGAGCCAUCACCAGCGACUAUGUCGUAG |
| | | UGGGUAAAGCUCACUUGAGGAUCACCCAUGUGAGCAUCAAAG |
| 40943 | 305 | ACUGGCGCUUUUAUCUGAUUACUUUGAGAGCCAUCACCAGCGACUAUGUCGUAG |
| | | UGGGUAAAGCUCACCUGAGGAUCACCCAUGUGAGCAUCAAAG |
| 40944 | 306 | ACUGGCGCUUUUAUCUGAUUACUUUGAGAGCCAUCACCAGCGACUAUGUCGUAG |
| | | UGGGUAAAGCUCACAUGAGGAUCACCUAUGUGAGCAUCAAAG |
| 40945 | 307 | ACUGGCGCUUUUAUCUGAUUACUUUGAGAGCCAUCACCAGCGACUAUGUCGUAG |
| | | UGGGUAAAGCUCACAUUAGGAUCACCAAUGUGAGCAUCAAAG |
| 40946 | 308 | ACUGGCGCUUUUAUCUGAUUACUUUGAGAGCCAUCACCAGCGACUAUGUCGUAG |
| | | UGGGUAAAGCUCACAUUAGGAUCACCGAUGUGAGCAUCAAAG |
| 40947 | 309 | ACUGGCGCUUUUAUCUGAUUACUUUGAGAGCCAUCACCAGCGACUAUGUCGUAG |
| | | UGGGUAAAGCUCACAUUAGGAUCACCUAUGUGAGCAUCAAAG |
| 40948 | 310 | ACUGGCGCUUUUAUCUGAUUACUUUGAGAGCCAUCACCAGCGACUAUGUCGUAG |
| | | UGGGUAAAGCUCACAUGAGGAUUACCCAUGUGAGCAUCAAAG |
| 40949 | 311 | ACUGGCGCUUUUAUCUGAUUACUUUGAGAGCCAUCACCAGCGACUAUGUCGUAG |
| | | UGGGUAAAGCUCACAUGAGGAUAACCCAUGUGAGCAUCAAAG |
| 40950 | 312 | ACUGGCGCUUUUAUCUGAUUACUUUGAGAGCCAUCACCAGCGACUAUGUCGUAG |
| | | UGGGUAAAGCUCACAUGAGGAUGACCCAUGUGAGCAUCAAAG |
| 40951 | 313 | ACUGGCGCUUUUAUCUGAUUACUUUGAGAGCCAUCACCAGCGACUAUGUCGUAG |
| | | UGGGUAAAGCUCACAUGAGGACCACCCAUGUGAGCAUCAAAG |
| 40952 | 314 | ACUGGCGCUUUUAUCUGAUUACUUUGAGAGCCAUCACCAGCGACUAUGUCGUAG |
| | | UGGGUAAAGCUCAGAUGAGGAUCACCCAUGGGAGCAUCAAAG |
| 40953 | 315 | ACUGGCGCUUUUAUCUGAUUACUUUGAGAGCCAUCACCAGCGACUAUGUCGUAG |
| | | UGGGUAAAGCUCACAUGGGGAUCACCCAUGUGAGCAUCAAAG |
| 40954 | 317 | ACUGGCGCUUCUAUCUGAUUACUCUGAGCGCCAUCACCAGCGACUAUGUCGUAG |
| | | UGGGUAAAGCUCACAUGAGGAUCACCCAUGUGAGCAUCAGAG |
| 40955 | 318 | ACUGGCGCUUCUAUCUGAUUACUCUGAGCGCCAUCACCAGCGACUAUGUCGUAG |
| | | UGGGUAAAGCUGCACUAUGGGCGCAGCGUCAAUGACGCUGACGGUACAGGCCAC |
| | | AUGAGGAUCACCCAUGUGGUAUAGUGCAGCAUCAGAG |
| 40956 | 319 | ACUGGCGCUUCUAUCUGAUUACUCUGAGCGCCAUCACCAGCGACUAUGUCGUAG |
| | | UGGGUAAAGCUGCACUAUGGGCGCAGCUCAUGAGGAUCACCCAUGAGCUGACGG |
| | | UACAGGCCACAUGAGGAUCACCCAUGUGGUAUAGUGCAGCAUCAGAG |
| 40957 | 320 | ACUGGCGCUUCUAUCUGAUUACUCUGAGCGCCAUCACCAGCGACUAUGUCGUAG |
| | | UGGGUAAAGCUGCACUAUGGGCGCAGACAUGGCAGUCGUAACGACGCGGGUCUG |
| | | ACGGUACAGGCCACAUGAGGAUCACCCAUGUGGUAUAGUGCAGCAUCAGAG |
| 40958 | 321 | ACUGGCGCUUUUAUCUGAUUACUUUGAGAGCCAUCACCAGCGACUAUGUCGUAG |
| | | UGGGUAAAGCUGCACUAUGGGGCCACAUGAGGAUCACCCAUGUGGUGUACAGCG |
| | | CAGCGUCAAUGACGCUGACGAUAGUGCAGCAUCAAAG |

In some embodiments, a sgRNA variant comprises one or more additional modifications to a sequence of SEQ ID NO:2238, SEQ ID NO:2239, SEQ ID NO:2240, SEQ ID NO:2241, SEQ ID NO:2243, SEQ ID NO:2256, SEQ ID NO:2274, SEQ ID NO:2275, SEQ ID NO:2279, SEQ ID NO:2281, SEQ ID NO: 2285, SEQ ID NO: 39984, SEQ ID NO: 39987, or SEQ ID NO: 40003 of Table 2.

In some embodiments of the gRNA variants of the disclosure, the gRNA variant comprises at least one modification compared to the reference guide scaffold of SEQ ID NO:5, wherein the at least one modification is selected from one or more of: (a) a C18G substitution in the triplex loop; (b) a G55 insertion in the stem bubble; (c) a U1 deletion; (d) a modification of the extended stem loop wherein (i) a 6 nt loop and 13 loop-proximal base pairs are replaced by a Uvsx hairpin; and (ii) a deletion of A99 and a substitution of G65U that results in a loop-distal base that is fully base-paired.

In some embodiments, a gRNA variant comprises an exogenous stem loop having a long non-coding RNA (lncRNA). As used herein, a lncRNA refers to a non-coding RNA that is longer than approximately 200 bp in length. In some embodiments, the 5′ and 3′ ends of the exogenous stem loop are base paired; i.e., interact to form a region of duplex RNA. In some embodiments, the 5′ and 3′ ends of the exogenous stem loop are base paired, and one or more regions between the 5′ and 3′ ends of the exogenous stem loop are not base paired, forming the loop.

In some embodiments, the disclosure provide gRNA variants with nucleotide modifications relative to reference gRNA having: (a) substitution of 1 to 15 consecutive or non-consecutive nucleotides in the gRNA variant in one or more regions; (b) a deletion of 1 to 10 consecutive or non-consecutive nucleotides in the gRNA variant in one or more regions; (c) an insertion of 1 to 10 consecutive or non-consecutive nucleotides in the gRNA variant in one or more regions; (d) a substitution of the scaffold stem loop or the extended stem loop with an RNA stem loop sequence from a heterologous RNA source with proximal 5′ and 3′ ends; or any combination of (a)-(d). Any of the substitutions, insertions and deletions described herein can be combined to generate a gRNA variant of the disclosure. For example, a gRNA variant can comprise at least one substitution and at least one deletion relative to a reference gRNA, at least one substitution and at least one insertion relative to a reference gRNA, at least one insertion and at least one deletion relative to a reference gRNA, or at least one substitution, one insertion and one deletion relative to a reference gRNA.

In some embodiments, a sgRNA variant of the disclosure comprises one or more modifications to the sequence of a previously generated variant, the previously generated variant itself serving as the sequence to be modified. In some cases, one or modifications are introduced to the pseudoknot region of the scaffold. In other cases, one or modifications are introduced to the triplex region of the scaffold. In other cases, one or modifications are introduced to the scaffold bubble. In other cases, one or modifications are introduced to the extended stem region of the scaffold. In still other cases, one of modifications are introduced into two or more of the foregoing regions. Such modifications can comprise an insertion, deletion, or substitution of one or more nucleotides in the foregoing regions, or any combination thereof. Exemplary methods to generate and assess the modifications are described in Example 20.

In some embodiments, a sgRNA variant comprises one or more modifications to a sequence of SEQ ID NO: 2238, SEQ ID NO: 2239, SEQ ID NO: 2240, SEQ ID NO: 2241, SEQ ID NO:2241, SEQ ID NO:2274, SEQ ID NO:2275, SEQ ID NO: 2279, or SEQ ID NO: 2285, SEQ ID NO: 39984, SEQ ID NO: 39987, or SEQ ID NO: 40003.

In exemplary embodiments, a gRNA variant comprises one or more modifications relative to gRNA scaffold variant 174 (SEQ ID NO:2238), wherein the resulting gRNA variant exhibits a improved functional characteristic compared to the parent 174, when assessed in an in vitro or in vivo assay under comparable conditions. In other exemplary embodiments, a gRNA variant comprises one or more modifications relative to gRNA scaffold variant 175 (SEQ ID NO:2239), wherein the resulting gRNA variant exhibits a improved functional characteristic compared to the parent 175, when assessed in an in vitro or in vivo assay under comparable conditions. For example, variants with modifications to the triplex loop of gRNA variant 175 show high enrichment relative to the 175 scaffold, particularly mutations to C15 or C17. Additionally, changes to either member of the predicted pair in the pseudoknot stem between G7 and A29 are both highly enriched relative to the 175 scaffold, with converting A29 to a C or a T to form a canonical Watson-Crick pairing (G7:C29), and the second of which would form a GU wobble pair (G7:U29), both of which may be expected to increase stability of the helix relative to the G:A pair. In addition, the insertion of a C at position 54 in guide scaffold 175 results in an enriched modification.

In some embodiments, the disclosure provides gRNA variants comprising one or more modifications to the gRNA scaffold variant 174 (SEQ ID NO: 2238) selected from the group consisting of the modifications of Table 28, wherein the resulting gRNA variant exhibits an improved functional characteristic compared to the parent 174, when assessed in an in vitro or in vivo assay under comparable conditions. In some embodiments, the improved functional characteristic is one or more functional properties selected from the group consisting of increased editing activity, increased pseudoknot stem stability, increased triplex region stability, increased scaffold stem stability, extended stem stability, reduced off-target folding intermediates, and increased binding affinity to a Class 2, Type V CRISPR protein. In the foregoing embodiments, the gRNA comprising one or more modifications to the gRNA scaffold variant 174 selected from the group consisting of the modifications of Table 28 (with a linked targeting sequence and complexed with a Class 2, Type V CRISPR protein) exhibits an improved enrichment score (log₂) of at least about 2.0, at least about 2.5, at least about 3, or at least about 3.5 greater compared to the score of the gRNA scaffold of SEQ ID NO: 2238 in an in vitro assay.

In some embodiments, the disclosure provides gRNA variants comprising one or more modifications to the gRNA scaffold variant 175 (SEQ ID NO: 2239) selected from the group consisting of the modifications of Table 29, wherein the resulting gRNA variant exhibits an improved functional characteristic compared to the parent 175, when assessed in an in vitro or in vivo assay under comparable conditions. In some embodiments, the improved functional characteristic is one or more functional properties selected from the group consisting of increased editing activity, increased pseudoknot stem stability, increased triplex region stability, increased scaffold stem stability, extended stem stability, reduced off-target folding intermediates, and increased binding affinity to a Class 2, Type V CRISPR protein. In the foregoing embodiments, the gRNA comprising one or more modifications to the gRNA scaffold variant 175 selected from the group consisting of the modifications of Table 29 (with a linked targeting sequence and complexed with a Class 2, Type V CRISPR protein) exhibits an improved enrichment score (log₂) of at least about 1.2, at least about 1.5, at least about 2.0, at least about 2.5, at least about 3, or at least about 3.5 greater compared to the score of the gRNA scaffold of SEQ ID NO: 2239 in an in vitro assay.

In a particular embodiment, the one or more modifications of gRNA scaffold variant 174 are selected from the group consisting of nucleotide positions U11, U24, A29, U65, C66, C68, A69, U76, G77, A79, and A87. In a particular embodiment, the modifications of gRNA scaffold variant 174 are U11C, U24C, A29C, U65C, C66G, C68U, an insertion of ACGGA at position 69, an insertion of UCCGU at position 76, G77A, an insertion of GA at position 79, A87G. In another particular embodiment, the modifications of gRNA scaffold variant 175 are selected from the group consisting of nucleotide positions C9, U11, C17, U24, A29, G54, C65, A89, and A96. In a particular embodiment, the modifications of gRNA scaffold variant 174 are C9U, U11C, C17G, U24C, A29C, an insertion of G at position 54, an insertion of C at position 65, A89G, and A96G.

In exemplary embodiments, a gRNA variant comprises one or more modifications relative to gRNA scaffold variant 215 (SEQ ID NO:2275), wherein the resulting gRNA variant exhibits an improved functional characteristic compared to the parent 215, when assessed in an in vitro or in vivo assay under comparable conditions.

In exemplary embodiments, a gRNA variant comprises one or more modifications relative to gRNA scaffold variant 221 (SEQ ID NO: 2281), wherein the resulting gRNA variant exhibits an improved functional characteristic compared to the parent 221, when assessed in an in vitro or in vivo assay under comparable conditions.

In exemplary embodiments, a gRNA variant comprises one or more modifications relative to gRNA scaffold variant 225 (SEQ ID NO: 2285), wherein the resulting gRNA variant exhibits an improved functional characteristic compared to the parent 225, when assessed in an in vitro or in vivo assay under comparable conditions.

In exemplary embodiments, a gRNA variant comprises one or more modifications relative to gRNA scaffold variant 235 (SEQ ID NO: 39987), wherein the resulting gRNA variant exhibits an improved functional characteristic compared to the parent 225, when assessed in an in vitro or in vivo assay under comparable conditions.

In exemplary embodiments, a gRNA variant comprises one or more modifications relative to gRNA scaffold variant 251 (SEQ ID NO: 40003), wherein the resulting gRNA variant exhibits an improved functional characteristic compared to the parent 251, when assessed in an in vitro or in vivo assay under comparable conditions.

In the foregoing embodiments, the improved functional characteristic includes, but is not limited to one or more of increased stability, increased transcription of the gRNA, increased resistance to nuclease activity, increased folding rate of the gRNA, decreased side product formation during folding, increased productive folding, increased binding affinity to a CasX protein, increased binding affinity to a target nucleic acid when complexed with the CasX protein, increased gene editing when complexed with the CasX protein, increased specificity of editing when complexed with the CasX protein, decreased off-target editing when complexed with the CasX protein, and increased ability to utilize a greater spectrum of one or more PAM sequences, including ATC, CTC, GTC, or TTC, in the modifying of target nucleic acid when complexed with the CasX protein. In some cases, the one or more of the improved characteristics of the gRNA variant is at least about 1.1 to about 100,000-fold improved relative to the gRNA from which it was derived. In other cases, the one or more improved characteristics of the gRNA variant is at least about 1.1, at least about 10, at least about 100, at least about 1000, at least about 10,000, at least about 100,000-fold or more improved relative to the gRNA from which it was derived. In other cases, the one or more of the improved characteristics of the gRNA variant is about 1.1 to 100,00-fold, about 1.1 to 10,00-fold, about 1.1 to 1,000-fold, about 1.1 to 500-fold, about 1.1 to 100-fold, about 1.1 to 50-fold, about 1.1 to 20-fold, about 10 to 100,00-fold, about 10 to 10,00-fold, about 10 to 1,000-fold, about 10 to 500-fold, about 10 to 100-fold, about 10 to 50-fold, about 10 to 20-fold, about 2 to 70-fold, about 2 to 50-fold, about 2 to 30-fold, about 2 to 20-fold, about 2 to 10-fold, about 5 to 50-fold, about 5 to 30-fold, about 5 to 10-fold, about 100 to 100,00-fold, about 100 to 10,00-fold, about 100 to 1,000-fold, about 100 to 500-fold, about 500 to 100,00-fold, about 500 to 10,00-fold, about 500 to 1,000-fold, about 500 to 750-fold, about 1,000 to 100,00-fold, about 10,000 to 100,00-fold, about 20 to 500-fold, about 20 to 250-fold, about 20 to 200-fold, about 20 to 100-fold, about 20 to 50-fold, about 50 to 10,000-fold, about 50 to 1,000-fold, about 50 to 500-fold, about 50 to 200-fold, or about 50 to 100-fold, improved relative to the gRNA from which it was derived. In other cases, the one or more improved characteristics of the gRNA variant is about 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 25-fold, 30-fold, 40-fold, 45-fold, 50-fold, 55-fold, 60-fold, 70-fold, 80-fold, 90-fold, 100-fold, 110-fold, 120-fold, 130-fold, 140-fold, 150-fold, 160-fold, 170-fold, 180-fold, 190-fold, 200-fold, 210-fold, 220-fold, 230-fold, 240-fold, 250-fold, 260-fold, 270-fold, 280-fold, 290-fold, 300-fold, 310-fold, 320-fold, 330-fold, 340-fold, 350-fold, 360-fold, 370-fold, 380-fold, 390-fold, 400-fold, 425-fold, 450-fold, 475-fold, or 500-fold improved relative to the gRNA from which it was derived.

In some embodiments, the gRNA variant comprises an exogenous extended stem loop, with such differences from a reference gRNA described as follows. In some embodiments, an exogenous extended stem loop has little or no identity to the reference stem loop regions disclosed herein (e.g., SEQ ID NO:15). In some embodiments, an exogenous stem loop is at least 10 bp, at least 20 bp, at least 30 bp, at least 40 bp, at least 50 bp, at least 60 bp, at least 70 bp, at least 80 bp, at least 90 bp, at least 100 bp, at least 200 bp, at least 300 bp, at least 400 bp, at least 500 bp, at least 600 bp, at least 700 bp, at least 800 bp, at least 900 bp, at least 1,000 bp, at least 2,000 bp, at least 3,000 bp, at least 4,000 bp, at least 5,000 bp, at least 6,000 bp, at least 7,000 bp, at least 8,000 bp, at least 9,000 bp, at least 10,000 bp, at least 12,000 bp, at least 15,000 bp or at least 20,000 bp. In some embodiments, the gRNA variant comprises an extended stem loop region comprising at least 10, at least 100, at least 500, at least 1000, or at least 10,000 nucleotides. In some embodiments, the heterologous stem loop increases the stability of the gRNA. In some embodiments, the heterologous RNA stem loop is capable of binding a protein, an RNA structure, a DNA sequence, or a small molecule. In some embodiments, an exogenous stem loop region replacing the stem loop comprises an RNA stem loop or hairpin in which the resulting gRNA has increased stability and, depending on the choice of loop, can interact with certain cellular proteins or RNA. Such exogenous extended stem loops can comprise, for example a thermostable RNA such as MS2 hairpin (ACAUGAGGAUCACCCAUGU (SEQ ID NO: 35)), QP hairpin (UGCAUGUCUAAGACAGCA (SEQ ID NO: 36)), U1 hairpin II (AAUCCAUUGCACUCCGGAUU (SEQ ID NO: 37)), Uvsx (CCUCUUCGGAGG (SEQ ID NO: 38)), PP7 hairpin (AGGAGUUUCUAUGGAAACCCU (SEQ ID NO: 39)), Phage replication loop (AGGUGGGACGACCUCUCGGUCGUCCUAUCU (SEQ ID NO: 40)), Kissing loop_a (UGCUCGCUCCGUUCGAGCA (SEQ ID NO: 41)), Kissing loop_b1 (UGCUCGACGCGUCCUCGAGCA (SEQ ID NO: 42)), Kissing loop_b2 (UGCUCGUUUGCGGCUACGAGCA (SEQ ID NO: 43)), G quadriplex M3q (AGGGAGGGAGGGAGAGG (SEQ ID NO: 44)), G quadriplex telomere basket (GGUUAGGGUUAGGGUUAGG (SEQ ID NO: 45)), Sarcin-ricin loop (CUGCUCAGUACGAGAGGAACCGCAG (SEQ ID NO: 46)) or Pseudoknots (UACACUGGGAUCGCUGAAUUAGAGAUCGGCGUCCUUUCAUUCUAUAUACUUUGG AGUUUUAAAAUGUCUCUAAGUACA (SEQ ID NO: 47)). In some embodiments, the extended stem loop comprises UGGGCGCAGCGUCAAUGACGCUGACGGUACA (Stem IIB; SEQ ID NO: 41843), GCACUAUGGGCGCAGCGUCAAUGACGCUGACGGUACAGGCCAGACAAUUAUUGU CUGGUAUAGUGC (Stem II; SEQ ID NO: 41844), CAGGAAGCACUAUGGGCGCAGCGUCAAUGACGCUGACGGUACAGGCCAGACAAU UAUUGUCUGGUAUAGUGCAGCAGCAGAACAAUUUGCUGAGGGCUAUUGAGGCGC AACAGCAUCUGUUGCAACUCACAGUCUGGGGCAUCAAGCAGCUCCAGGCAAGAA UCCUG (Stem II-V SEQ ID NO: 41845), GCUGACGGUACAGGC (RBE; SEQ ID NO: 41846), and AGGAGCUUUGUUCCUUGGGUUCUUGGGAGCAGCAGGAAGCACUAUGGGCGCAGC GUCAAUGACGCUGACGGUACAGGCCAGACAAUUAUUGUCUGGUAUAGUGCAGCA GCAGAACAAUUUGCUGAGGGCUAUUGAGGCGCAACAGCAUCUGUUGCAACUCAC AGUCUGGGGCAUCAAGCAGCUCCAGGCAAGAAUCCUGGCUGUGGAAAGAUACCU AAAGGAUCAACAGCUCCU (full-length RRE; SEQ ID NO: 41847).

In some embodiments, a gRNA variant comprises a terminal fusion partner. The term gRNA variant is inclusive of variants that include exogenous sequences such as terminal fusions, or internal insertions. Exemplary terminal fusions may include fusion of the gRNA to a self-cleaving ribozyme or protein binding motif. As used herein, a “ribozyme” refers to an RNA or segment thereof with one or more catalytic activities similar to a protein enzyme. Exemplary ribozyme catalytic activities may include, for example, cleavage and/or ligation of RNA, cleavage and/or ligation of DNA, or peptide bond formation. In some embodiments, such fusions could either improve scaffold folding or recruit DNA repair machinery. For example, a gRNA may in some embodiments be fused to a hepatitis delta virus (HDV) antigenomic ribozyme, HDV genomic ribozyme, hatchet ribozyme (from metagenomic data), env25 pistol ribozyme (representative from Aliistipes putredinis), HH15 Minimal Hammerhead ribozyme, tobacco ringspot virus (TRSV) ribozyme, WT viral Hammerhead ribozyme (and rational variants), or Twisted Sister 1 or RBMX recruiting motif. Hammerhead ribozymes are RNA motifs that catalyze reversible cleavage and ligation reactions at a specific site within an RNA molecule. Hammerhead ribozymes include type I, type II and type III hammerhead ribozymes. The HDV, pistol, and hatchet ribozymes have self-cleaving activities. gRNA variants comprising one or more ribozymes may allow for expanded gRNA function as compared to a gRNA reference. For example, gRNAs comprising self-cleaving ribozymes can, in some embodiments, be transcribed and processed into mature gRNAs as part of polycistronic transcripts. Such fusions may occur at either the 5′ or the 3′ end of the gRNA. In some embodiments, a gRNA variant comprises a fusion at both the 5′ and the 3′ end, wherein each fusion is independently as described herein.

In the embodiments of the gRNA variants, the gRNA variant further comprises a spacer (or targeting sequence) region located at the 3′ end of the gRNA, capable of hybridizing with a target nucleic acid which comprises at least 14 to about 35 nucleotides wherein the spacer is designed with a sequence that is complementary to a target nucleic acid. In some embodiments, the encoded gRNA variant comprises a targeting sequence of at least 10 to 20 nucleotides complementary to a target nucleic acid. In some embodiments, the targeting sequence has 14, 15, 16, 17, 18, 19, 20, 21, 22, 23, 24, 25, 26, 27, 28, 29, or 30 nucleotides. In some embodiments, the encoded gRNA variant comprises a targeting sequence having 20 nucleotides. In some embodiments, the targeting sequence has 25 nucleotides. In some embodiments, the targeting sequence has 24 nucleotides. In some embodiments, the targeting sequence has 23 nucleotides. In some embodiments, the targeting sequence has 22 nucleotides. In some embodiments, the targeting sequence has 21 nucleotides. In some embodiments, the targeting sequence has 20 nucleotides. In some embodiments, the targeting sequence has 19 nucleotides. In some embodiments, the targeting sequence has 18 nucleotides. In some embodiments, the targeting sequence has 17 nucleotides. In some embodiments, the targeting sequence has 16 nucleotides. In some embodiments, the targeting sequence has 15 nucleotides. In some embodiments, the targeting sequence has 14 nucleotides.

h. Complex Formation with CasX Protein

In some embodiments, a gRNA variant has an improved ability to form an RNP complex with a Class 2, Type V protein, including CasX variant proteins comprising any one of the sequences SEQ ID NOS: 49-160, 40208-40369, or 40828-40912 of Table 3, or a sequence having at least about 50%, at least about 60%, at least about 70%, at least about 80%, at least about 85%, at least about 90%, at least about 91%, at least about 92%, at least about 93%, at least about 94%, at least about 95%, at least about 96%, at least about 97%, at least about 98%, or at least about 99% identity thereto. In some embodiments, upon expression, the gRNA variant is complexed as an RNP with a CasX variant protein comprising any one of the sequences SEQ ID NOS: 49-160, 40208-40369, or 40828-40912 of Table 3, or a sequence having at least about 50%, at least about 60%, at least about 70%, at least about 80%, at least about 85%, at least about 90%, at least about 91%, at least about 92%, at least about 93%, at least about 94%, at least about 95%, at least about 96%, at least about 97%, at least about 98%, or at least about 99% identity thereto. In some embodiments, upon expression, the gRNA variant is complexed as an RNP with a CasX variant protein comprising any one of the sequences SEQ ID NOS: 85-160, 40208-40369, or 40828-40912, or a sequence having at least about 50%, at least about 60%, at least about 70%, at least about 80%, at least about 85%, at least about 90%, at least about 91%, at least about 92%, at least about 93%, at least about 94%, at least about 95%, at least about 96%, at least about 97%, at least about 98%, or at least about 99% identity thereto.

In some embodiments, a gRNA variant has an improved ability to form a complex with a CasX variant protein when compared to a reference gRNA, thereby improving its ability to form a cleavage-competent ribonucleoprotein (RNP) complex with the CasX protein, as described in the Examples. Improving ribonucleoprotein complex formation may, in some embodiments, improve the efficiency with which functional RNPs are assembled. In some embodiments, greater than 90%, greater than 93%, greater than 95%, greater than 96%, greater than 97%, greater than 98% or greater than 99% of RNPs comprising a gRNA variant and its targeting sequence are competent for gene editing of a target nucleic acid.

Exemplary nucleotide changes that can improve the ability of gRNA variants to form a complex with CasX protein may, in some embodiments, include replacing the scaffold stem with a thermostable stem loop. Without wishing to be bound by any theory, replacing the scaffold stem with a thermostable stem loop could increase the overall binding stability of the gRNA variant with the CasX protein. Alternatively, or in addition, removing a large section of the stem loop could change the gRNA variant folding kinetics and make a functional folded gRNA easier and quicker to structurally-assemble, for example by lessening the degree to which the gRNA variant can get “tangled” in itself. In some embodiments, choice of scaffold stem loop sequence could change with different spacers that are utilized for the gRNA. In some embodiments, scaffold sequence can be tailored to the spacer and therefore the target sequence. Biochemical assays can be used to evaluate the binding affinity of CasX protein for the gRNA variant to form the RNP, including the assays of the Examples. For example, a person of ordinary skill can measure changes in the amount of a fluorescently tagged gRNA that is bound to an immobilized CasX protein, as a response to increasing concentrations of an additional unlabeled “cold competitor” gRNA. Alternatively, or in addition, fluorescence signal can be monitored to or seeing how it changes as different amounts of fluorescently labeled gRNA are flowed over immobilized CasX protein. Alternatively, the ability to form an RNP can be assessed using in vitro cleavage assays against a defined target nucleic acid sequence.

### IV. CRISPR Proteins of the AAV Systems

The present disclosure provides AAV systems encoding a CRISPR nuclease that have utility in genome editing of eukaryotic cells, as well as being an integral component of the self-inactivating feature of the construct. In some embodiments, the CRISPR nuclease employed in the genome editing systems is a Class 2, Type V nuclease. Although members of Class 2, Type V CRISPR-Cas systems have differences, they share some common characteristics that distinguish them from the Cas9 systems. Firstly, the Class 2, Type V nucleases possess a single RNA-guided RuvC domain-containing effector but no HNH domain, and they recognize T-rich PAM 5′ upstream to the target region on the non-targeted strand, which is different from Cas9 systems which rely on G-rich PAM at 3′ side of target sequences. Type V nucleases generate staggered double-stranded breaks distal to the PAM sequence, unlike Cas9, which generates a blunt end in the proximal site close to the PAM. In addition, Type V nucleases degrade ssDNA in trans when activated by target dsDNA or ssDNA binding in cis. In some embodiments, the Type V nucleases of the embodiments recognize a 5′-TC PAM motif and produce staggered ends cleaved solely by the RuvC domain. In some embodiments, the Type V nuclease is selected from the group consisting of Cas12a, Cas12b, Cas12c, Cas12d (CasY), Cas12j, Cas12k, CasPhi, C2c4, C2c8, C2c5, C2c10, C2c9, CasZ and CasX. In some embodiments, the present disclosure provides AAV systems encoding a CasX variant protein and one or more gRNA acids that upon expression in a transfected cell are able to form an RNP complex and are specifically designed to modify a target nucleic acid sequence in eukaryotic cells, as well as cleave the self-inactivating segments incorporated into the polynucleotide comprising the transgene of the AAV construct.

The term “CasX protein”, as used herein, refers to a family of proteins, and encompasses all naturally occurring CasX proteins, proteins that share at least 50% identity to naturally occurring CasX proteins, as well as CasX variants possessing one or more improved characteristics relative to a naturally-occurring reference CasX protein, described more fully, below.

CasX proteins of the disclosure comprise at least one of the following domains: a non-target strand binding (NTSB) domain, a target strand loading (TSL) domain, a helical I domain (which is further divided into helical I-I and I-II subdomains), a helical II domain, an oligonucleotide binding domain (OBD, which is further divided into OBD-I and OBD-II subdomains), and a RuvC DNA cleavage domain (which is further divided into RuvC-I and II subdomains). The RuvC domain may be modified or deleted in a catalytically-dead CasX variant, described more fully, below.

In some embodiments, a CasX variant protein can bind and/or modify (e.g., nick, catalyze a double-strand break, methylate, demethylate, etc.) a target nucleic acid at a specific sequence targeted by an associated gRNA, which hybridizes to a sequence within the target nucleic acid sequence.

a. Reference CasX Proteins

The disclosure provides naturally-occurring CasX proteins (referred to herein as a “reference CasX protein”), which were subsequently modified to create the CasX variants of the disclosure. For example, reference CasX proteins can be isolated from naturally occurring prokaryotes, such as Deltaproteobacteria, Planctomycetes, or Candidatus Sungbacteria species. A reference CasX protein is a type II CRISPR/Cas endonuclease belonging to the CasX (interchangeably referred to as Cas12e) family of proteins that interacts with a guide RNA to form a ribonucleoprotein (RNP) complex.

In some cases, a reference CasX protein is isolated or derived from Deltaproteobacter. In some embodiments, a reference CasX protein comprises a sequence identical to a sequence of:

| (SEQ ID NO: 1) | |
|---|---|
| 1 | MEKRINKIRK KLSADNATKP VSRSGPMKTL LVRVMTDDLK KRLEKRRKKP EVMPQVISNN |
| 61 | AANNLRMLLD DYTKMKEAIL QVYWQEFKDD HVGLMCKFAQ PASKKIDQNK LKPEMDEKGN |
| 121 | LTTAGFACSQ CGQPLFVYKL EQVSEKGKAY TNYFGRCNVA EHEKLILLAQ LKPEKDSDEA |
| 181 | VTYSLGKFGQ RALDFYSIHV TKESTHPVKP LAQIAGNRYA SGPVGKALSD ACMGTIASFL |
| 241 | SKYQDIIIEH QKVVKGNQKR LESLRELAGK ENLEYPSVTL PPQPHTKEGV DAYNEVIARV |
| 301 | RMWVNLNLWQ KLKLSRDDAK PLLRLKGFPS FPVVERRENE VDWWNTINEV KKLIDAKRDM |
| 361 | GRVFWSGVTA EKRNTILEGY NYLPNENDHK KREGSLENPK KPAKRQFGDL LLYLEKKYAG |
| 421 | DWGKVFDEAW ERIDKKIAGL TSHIEREEAR NAEDAQSKAV LTDWLRAKAS FVLERLKEMD |
| 481 | EKEFYACEIQ LQKWYGDLRG NPFAVEAENR VVDISGFSIG SDGHSIQYRN LLAWKYLENG |
| 541 | KREFYLLMNY GKKGRIRFTD GTDIKKSGKW QGLLYGGGKA KVIDLTFDPD DEQLIILPLA |
| 601 | FGTRQGREFI WNDLLSLETG LIKLANGRVI EKTIYNKKIG RDEPALFVAL TFERREVVDP |
| 661 | SNIKPVNLIG VDRGENIPAV IALTDPEGCP LPEFKDSSGG PTDILRIGEG YKEKQRAIQA |
| 721 | AKEVEQRRAG GYSRKFASKS RNLADDMVRN SARDLFYHAV THDAVLVFEN LSRGFGRQGK |
| 781 | RTFMTERQYT KMEDWLTAKL AYEGLTSKTY LSKTLAQYTS KTCSNCGFTI TTADYDGMLV |
| 841 | RLKKTSDGWA TTLNNKELKA EGQITYYNRY KRQTVEKELS AELDRLSEES GNNDISKWTK |
| 901 | GRRDEALFLL KKRFSHRPVQ EQFVCLDCGH EVHADEQAAL NIARSWLFLN SNSTEFKSYK |
| 961 | SGKQPFVGAW QAFYKRRLKE VWKPNA. |

In some cases, a reference CasX protein is isolated or derived from Planctomycetes. In some embodiments, a reference CasX protein comprises a sequence identical to a sequence of:

| (SEQ ID NO: 2) | |
|---|---|
| 1 | MQEIKRINKI RRRLVKDSNT KKAGKTGPMK TLLVRVMTPD LRERLENLRK KPENIPQPIS |
| 61 | NTSRANLNKL LTDYTEMKKA ILHVYWEEFQ KDPVGLMSRV AQPAPKNIDQ RKLIPVKDGN |
| 121 | ERLTSSGFAC SQCCQPLYVY KLEQVNDKGK PHTNYFGRCN VSEHERLILL SPHKPEANDE |
| 181 | LVTYSLGKFG QRALDFYSIH VTRESNHPVK PLEQIGGNSC ASGPVGKALS DACMGAVASF |
| 241 | LTKYQDIILE HQKVIKKNEK RLANLKDIAS ANGLAFPKIT LPPQPHTKEG IEAYNNVVAQ |
| 301 | IVIWVNLNLW QKLKIGRDEA KPLQRLKGFP SFPLVERQAN EVDWWDMVCN VKKLINEKKE |
| 361 | DGKVFWQNLA GYKRQEALLP YLSSEEDRKK GKKFARYQFG DLLLHLEKKH GEDWGKVYDE |
| 421 | AWERIDKKVE GLSKHIKLEE ERRSEDAQSK AALTDWLRAK ASFVIEGLKE ADKDEFCRCE |
| 481 | LKLQKWYGDL RGKPFAIEAE NSILDISGFS KQYNCAFIWQ KDGVKKLNLY LIINYFKGGK |
| 541 | LRFKKIKPEA FEANRFYTVI NKKSGEIVPM EVNENFDDPN LIILPLAFGK RQGREFIWND |
| 601 | LLSLETGSLK LANGRVIEKT LYNRRTRQDE PALFVALTFE RREVLDSSNI KPMNLIGIDR |
| 661 | GENIPAVIAL TDPEGCPLSR FKDSLGNPTH ILRIGESYKE KQRTIQAAKE VEQRRAGGYS |
| 721 | RKYASKAKNL ADDMVRNTAR DLLYYAVTQD AMLIFENLSR GFGRQGKRTF MAERQYTRME |
| 781 | DWLTAKLAYE GLPSKTYLSK TLAQYTSKTC SNCGFTITSA DYDRVLEKLK KTATGWMTTI |
| 841 | NGKELKVEGQ ITYYNRYKRQ NVVKDLSVEL DRLSEESVNN DISSWTKGRS GEALSLLKKR |
| 901 | FSHRPVQEKF VCLNCGFETH ADEQAALNIA RSWLFLRSQE YKKYQTNKTT GNTDKRAFVE |
| 961 | TWQSFYRKKL KEVWKPAV. |

In some cases, a reference CasX protein is isolated or derived from Candidatus Sungbacteria. In some embodiments, a reference CasX protein comprises a sequence identical to a sequence of

| (SEQ ID NO: 3) | |
|---|---|
| 1 | MDNANKPSTK SLVNTTRISD HFGVTPGQVT RVFSFGIIPT KRQYAIIERW FAAVEAARER |
| 61 | LYGMLYAHFQ ENPPAYLKEK FSYETFFKGR PVLNGLRDID PTIMTSAVFT ALRHKAEGAM |
| 121 | AAFHTNHRRL FEEARKKMRE YAECLKANEA LLRGAADIDW DKIVNALRTR LNTCLAPEYD |
| 181 | AVIADFGALC AFRALIAETN ALKGAYNHAL NQMLPALVKV DEPEEAEESP RLRFENGRIN |
| 241 | DLPKFPVAER ETPPDTETII RQLEDMARVI PDTAEILGYI HRIRHKAARR KPGSAVPLPQ |
| 301 | RVALYCAIRM ERNPEEDPST VAGHELGEID RVCEKRRQGL VRTPEDSQIR ARYMDIISER |
| 361 | ATLAHPDRWT EIQFLRSNAA SRRVRAETIS APFEGFSWTS NRTNPAPQYG MALAKDANAP |
| 421 | ADAPELCICL SPSSAAFSVR EKGGDLIYMR PTGGRRGKDN PGKEITWVPG SFDEYPASGV |
| 481 | ALKLRLYFGR SQARRMLINK TWGLLSDNPR VFAANAELVG KKRNPQDRWK LFFHMVISGP |
| 541 | PPVEYLDESS DVRSRARTVI GINRGEVNPL AYAVVSVEDG QVLEEGLLGK KEYIDQLIET |
| 601 | RRRISEYQSR EQTPPRDLRQ RVRHLQDTVL GSARAKIHSL IAFWKGILAI ERLDDQFHGR |
| 661 | EQKIIPKKTY LANKTGFMNA LSFSGAVRVD KKGNPWGGMI EIYPGGISRT CTQCGTVWLA |
| 721 | RRPKNPGHRD AMVVIPDIVD DAAATGFDNV DCDAGTVDYG ELFTLSREWV RLTPRYSRVM |
| 781 | RGTLGDLERA IRQGDDRKSR QMLELALEPQ PQWGQFFCHR CGENGQSDVL AATNLARRAI |
| 841 | SLIRRLPDTD TPPTP. |

b. Class 2, Type V CasX Variant Proteins

The present disclosure provides Class 2, Type V, CasX variants of a reference CasX protein or variants derived from other CasX variants (interchangeably referred to herein as “Class 2, Type V CasX variant”, “CasX variant” or “CasX variant protein”), wherein the Class 2, Type V CasX variants comprise at least one modification in at least one domain relative to the reference CasX protein, including but not limited to the sequences of SEQ ID NOS:1-3, or at least one modification relative to another CasX variant. Any change in amino acid sequence of a reference CasX protein or to another CasX variant protein that leads to an improved characteristic of the CasX protein is considered a CasX variant protein of the disclosure. For example, CasX variants can comprise one or more amino acid substitutions, insertions, deletions, or swapped domains, or any combinations thereof, relative to a reference CasX protein sequence.

The CasX variants of the disclosure have one or more improved characteristics compared to a reference CasX protein of SEQ ID NO:1, SEQ ID NO:2 or SEQ ID NO:3, or the variant from which it was derived; e.g. CasX 491 (SEQ ID NO: 138) or CasX 515 (SEQ ID NO: 145). Exemplary improved characteristics of the CasX variant embodiments include, but are not limited to improved folding of the variant, increased binding affinity to the gRNA, increased binding affinity to the target nucleic acid, improved ability to utilize a greater spectrum of PAM sequences in the editing and/or binding of target nucleic acid, improved unwinding of the target DNA, increased editing activity, improved editing efficiency, improved editing specificity for the target nucleic acid, decreased off-target editing or cleavage, increased percentage of a eukaryotic genome that can be efficiently edited, increased activity of the nuclease, increased target strand loading for double strand cleavage, decreased target strand loading for single strand nicking, increased binding of the non-target strand of DNA, improved protein stability, improved protein:gRNA (RNP) complex stability, and improved fusion characteristics. In the foregoing embodiments, the one or more of the improved characteristics of the CasX variant is at least about 1.1 to about 100,000-fold improved relative to the reference CasX protein of SEQ ID NO: 1, SEQ ID NO: 2, or SEQ ID NO: 3, or CasX 491 (SEQ ID NO: 138) or CasX 515 (SEQ ID NO: 145), when assayed in a comparable fashion. In other embodiments, the improvement is at least about 1.1-fold, at least about 2-fold, at least about 5-fold, at least about 10-fold, at least about 50-fold, at least about 100-fold, at least about 500-fold, at least about 1000-fold, at least about 5000-fold, at least about 10,000-fold, or at least about 100,000-fold compared to the reference CasX protein of SEQ ID NO: 1, SEQ ID NO: 2, or SEQ ID NO: 3, or CasX 491 (SEQ ID NO: 138) or CasX 515 (SEQ ID NO: 145). when assayed in a comparable fashion. In other cases, the one or more improved characteristics of an RNP of the CasX variant and the gRNA variant are at least about 1.1, at least about 10, at least about 100, at least about 1000, at least about 10,000, at least about 100,000-fold or more improved relative to an RNP of the reference CasX protein of SEQ ID NO:1, SEQ ID NO:2, or SEQ ID NO:3 and the gRNA of Table 1 or CasX 491 or CasX 515 with gRNA 174. In other cases, the one or more of the improved characteristics of an RNP of the CasX variant and the gRNA variant are about 1.1 to 100,00-fold, about 1.1 to 10,00-fold, about 1.1 to 1,000-fold, about 1.1 to 500-fold, about 1.1 to 100-fold, about 1.1 to 50-fold, about 1.1 to 20-fold, about 10 to 100,00-fold, about 10 to 10,00-fold, about 10 to 1,000-fold, about 10 to 500-fold, about 10 to 100-fold, about 10 to 50-fold, about 10 to 20-fold, about 2 to 70-fold, about 2 to 50-fold, about 2 to 30-fold, about 2 to 20-fold, about 2 to 10-fold, about 5 to 50-fold, about 5 to 30-fold, about 5 to 10-fold, about 100 to 100,00-fold, about 100 to 10,00-fold, about 100 to 1,000-fold, about 100 to 500-fold, about 500 to 100,00-fold, about 500 to 10,00-fold, about 500 to 1,000-fold, about 500 to 750-fold, about 1,000 to 100,00-fold, about 10,000 to 100,00-fold, about 20 to 500-fold, about 20 to 250-fold, about 20 to 200-fold, about 20 to 100-fold, about 20 to 50-fold, about 50 to 10,000-fold, about 50 to 1,000-fold, about 50 to 500-fold, about 50 to 200-fold, or about 50 to 100-fold, improved relative to an RNP of the reference CasX protein of SEQ ID NO:1, SEQ ID NO:2, or SEQ ID NO:3 and the gRNA of Table 1, or CasX 491 or CasX 515 with gRNA 174, when assayed in a comparable fashion. In other cases, the one or more improved characteristics of an RNP of the CasX variant and the gRNA variant are about 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 25-fold, 30-fold, 40-fold, 45-fold, 50-fold, 55-fold, 60-fold, 70-fold, 80-fold, 90-fold, 100-fold, 110-fold, 120-fold, 130-fold, 140-fold, 150-fold, 160-fold, 170-fold, 180-fold, 190-fold, 200-fold, 210-fold, 220-fold, 230-fold, 240-fold, 250-fold, 260-fold, 270-fold, 280-fold, 290-fold, 300-fold, 310-fold, 320-fold, 330-fold, 340-fold, 350-fold, 360-fold, 370-fold, 380-fold, 390-fold, 400-fold, 425-fold, 450-fold, 475-fold, or 500-fold improved relative to an RNP of the reference CasX protein of SEQ ID NO:1, SEQ ID NO:2, or SEQ ID NO:3 and the gRNA of Table 1, or CasX 491 or CasX 515 with gRNA 174, when assayed in a comparable fashion.

In some embodiments, the modification of the CasX variant is a mutation in one or more amino acids of the reference CasX. In other embodiments, the modification is an insertion or substitution of a part or all of a domain from a different CasX protein. In a particular embodiment, the CasX variants of SEQ ID NOS: 144-160, 40208-40369, 40828-40912 have a NTSB and helical 1B domain of SEQ ID NO: 1, while the other domains are derived from SEQ ID NO: 2, in addition to individual modifications in select domains, described herein. Mutations can be introduced in any one or more domains of the reference CasX protein or in a CasX variant to result in a CasX variant, and may include, for example, deletion of part or all of one or more domains, or one or more amino acid substitutions, deletions, or insertions in any domain of the reference CasX protein or the CasX variant from which it was derived. The domains of CasX proteins include the non-target strand binding (NTSB) domain, the target strand loading (TSL) domain, the Helical I domain, the Helical II domain, the oligonucleotide binding domain (OBD), and the RuvC DNA cleavage domain. Without being bound to theory or mechanism, a NTSB domain in a CasX allows for binding to the non-target nucleic acid strand and may aid in unwinding of the non-target and target strands. The NTSB domain is presumed to be responsible for the unwinding, or the capture, of a non-target nucleic acid strand in the unwound state. An exemplary NTSB domain comprises amino acids 100-190 of SEQ ID NO: 1 or amino acids 102-191 of SEQ ID NO: 2. In some embodiments, the NTSB domain of a reference CasX protein comprises a four-stranded beta sheet. In some embodiments, the TSL acts to place or capture the target-strand in a folded state that places the scissile phosphate of the target strand DNA backbone in the RuvC active site. An exemplary TSL comprises amino acids 824-933 of SEQ ID NO: 1 or amino acids 811-920 of SEQ ID NO: 2. Without wishing to be bound by theory, it is thought that in some cases the Helical I domain may contribute to binding of the protospacer adjacent motif (PAM). In some embodiments, the Helical I domain of a reference CasX protein comprises one or more alpha helices. Exemplary Helical I-I and I-II domains comprise amino acids 56-99 and 191-331 of SEQ ID NO: 1, respectively, or amino acids 58-101 and 192-332 of SEQ ID NO: 2, respectively. The Helical II domain is responsible for binding to the guide RNA scaffold stem loop as well as the bound DNA. An exemplary Helical II domain comprises amino acids 332-508 of SEQ ID NO: 1, or amino acids 333-500 of SEQ ID NO: 2. The OBD largely binds the RNA triplex of the guide RNA scaffold. The OBD may also be responsible for binding to the protospacer adjacent motif (PAM). Exemplary OBD I and II domains comprise amino acids 1-55 and 509-659 of SEQ ID NO: 1, respectively, or amino acids 1-57 and 501-646 of SEQ ID NO: 2, respectively. The RuvC has a DED motif active site that is responsible for cleaving both strands of DNA (one by one, most likely the non-target strand first at 11-14 nucleotides (nt) into the targeted sequence and then the target strand next at 2-4 nucleotides after the target sequence, resulting in a staggered cut). Specifically in CasX, the RuvC domain is unique in that it is also responsible for binding the guide RNA scaffold stem loop that is critical for CasX function. Exemplary RuvC I and II domains comprise amino acids 660-823 and 934-986 of SEQ ID NO: 1, respectively, or amino acids 647-810 and 921-978 of SEQ ID NO: 2, respectively, while CasX variants may comprise mutations at positions 1658 and A708 relative to SEQ ID NO: 2, or the mutations of CasX 515, described below.

In some embodiments, the CasX variant protein comprises at least one modification in at least 1 domain, in at least each of 2 domains, in at least each of 3 domains, in at least each of 4 domains or in at least each of 5 domains of the reference CasX protein, including the sequences of SEQ ID NOS: 1-3. In some embodiments, the CasX variant protein comprises two or more modifications in at least one domain of the reference CasX protein. In some embodiments, the CasX variant protein comprises at least two modifications in at least one domain of the reference CasX protein, at least three modifications in at least one domain of the reference CasX protein or at least four or more modifications in at least one domain of the reference CasX protein. In some embodiments, wherein the CasX variant comprises two or more modifications compared to a reference CasX protein, and each modification is made in a domain independently selected from the group consisting of a NTSB, TSL, Helical I domain, Helical II domain, OBD, and RuvC DNA cleavage domain. In some embodiments, wherein the CasX variant comprises two or more modifications compared to a reference CasX protein, a modification is made in two or more domains. In some embodiments, the at least one modification of the CasX variant protein comprises a deletion of at least a portion of one domain of the reference CasX protein of SEQ ID NOS: 1-3. In some embodiments, the deletion is in the NTSB domain, TSL domain, Helical I domain, Helical II domain, OBD, or RuvC DNA cleavage domain.

In some cases, the CasX variants of the disclosure comprise modifications in structural regions that may encompass one or more domains. In some embodiments, a CasX variant comprises at least one modification of a region of non-contiguous amino acid residues of the CasX variant that form a channel in which gRNA:target nucleic acid complexing with the CasX variant occurs. In other embodiments, a CasX variant comprises at least one modification of a region of non-contiguous amino acid residues of the CasX variant that form an interface which binds with the gRNA. In other embodiments, a CasX variant comprises at least one modification of a region of non-contiguous amino acid residues of the CasX variant that form a channel which binds with the non-target strand DNA. In other embodiments, a CasX variant comprises at least one modification of a region of non-contiguous amino acid residues of the CasX variant that form an interface which binds with the protospacer adjacent motif (PAM) of the target nucleic acid. In other embodiments, a CasX variant comprises at least one modification of a region of non-contiguous surface-exposed amino acid residues of the CasX variant. In other embodiments, a CasX variant comprises at least one modification of a region of non-contiguous amino acid residues that form a core through hydrophobic packing in a domain of the CasX variant. In the foregoing embodiments of the paragraph, the modifications of the region can comprise one or more of a deletion, an insertion, or a substitution of one or more amino acids of the region; or between 2 to 15 amino acid residues of the region of the CasX variant are substituted with charged amino acids; or between 2 to 15 amino acid residues of a region of the CasX variant are substituted with polar amino acids; or between 2 to 15 amino acid residues of a region of the CasX variant are substituted with amino acids that stack, or have affinity with DNA or RNA bases.

In other embodiments, the disclosure provides CasX variants wherein the CasX variants comprise at least one modification relative to another CasX variant; e.g., CasX variant 515 and 527 is a variant of CasX variant 491 and CasX variants 668 and 672 are variants of CasX 535. In some embodiments, the at least one modification is selected from the group consisting of an amino acid insertion, deletion, or substitution. All variants that improve one or more functions or characteristics of the CasX variant protein when compared to a reference CasX protein or the variant from which it was derived described herein are envisaged as being within the scope of the disclosure. As described in the Examples, a CasX variant can be mutagenized to create another CasX variant. In a particular embodiment, the disclosure provides, in Example 21, Table 30, variants of CasX 515 (SEQ ID NO: 145) created by introducing modifications to the encoding sequence resulting in amino acid substitutions, deletions, or insertions at one or more positions in one or more domains.

Suitable mutagenesis methods for generating CasX variant proteins of the disclosure may include, for example, Deep Mutational Evolution (DME), deep mutational scanning (DMS), error prone PCR, cassette mutagenesis, random mutagenesis, staggered extension PCR, gene shuffling, or domain swapping (described in PCT/US20/36506 and WO2020247883A2, incorporated by reference herein). In some embodiments, the CasX variants are designed, for example by selecting multiple desired mutations in a CasX variant identified, for example, using the assays described in the Examples. In certain embodiments, the activity of a reference CasX or the CasX variant protein prior to mutagenesis is used as a benchmark against which the activity of one or more resulting CasX variants are compared, thereby measuring improvements in function of the new CasX variants.

In some embodiments of the CasX variants described herein, the at least one modification comprises: (a) a substitution of 1 to 100 consecutive or non-consecutive amino acids in the CasX variant compared to a reference CasX of SEQ ID NO:1, SEQ ID NO:2, SEQ ID NO:3, CasX variant 491 (SEQ ID NO: 138) or CasX variant 515 (SEQ ID NO: 145); (b) a deletion of 1 to 100 consecutive or non-consecutive amino acids in the CasX variant compared to a reference CasX or the variant from which it was derived; (c) an insertion of 1 to 100 consecutive or non-consecutive amino acids in the CasX compared to a reference CasX or the variant from which it was derived; or (d) any combination of (a)-(c). In some embodiments, the at least one modification comprises: (a) a substitution of 1-10 consecutive or non-consecutive amino acids in the CasX variant compared to a reference CasX of SEQ ID NO:1, SEQ ID NO:2, SEQ ID NO:3, or the variant from which it was derived; (b) a deletion of 1-5 consecutive or non-consecutive amino acids in the CasX variant compared to a reference CasX or the variant from which it was derived; (c) an insertion of 1-5 consecutive or non-consecutive amino acids in the CasX compared to a reference CasX or the variant from which it was derived; or (d) any combination of (a)-(c).

In some embodiments, the CasX variant protein comprises or consists of a sequence that has at least 1, at least 2, at least 3, at least 4, at least 5, at least 6, at least 7, at least 8, at least 9, at least 10, at least 20, at least 30, at least 40, at least 50, at least 60, at least 70, at lease 80, at least 90, or at least 100 alterations relative to the sequence of SEQ ID NO:1, SEQ ID NO:2, SEQ ID NO:3, CasX 491 or CasX 515. In some embodiments, the CasX variant protein comprises one more substitutions relative to CasX 491, or SEQ ID NO: 138. In some embodiments, the CasX variant protein comprises one more substitutions relative to CasX 515, or SEQ ID NO: 145. These alterations can be amino acid insertions, deletions, substitutions, or any combinations thereof. The alterations can be in one domain or in any domain or any combination of domains of the CasX variant. Any amino acid can be substituted for any other amino acid in the substitutions described herein. The substitution can be a conservative substitution (e.g., a basic amino acid is substituted for another basic amino acid). The substitution can be a non-conservative substitution (e.g., a basic amino acid is substituted for an acidic amino acid or vice versa). For example, a proline in a reference CasX protein can be substituted for any of arginine, histidine, lysine, aspartic acid, glutamic acid, serine, threonine, asparagine, glutamine, cysteine, glycine, alanine, isoleucine, leucine, methionine, phenylalanine, tryptophan, tyrosine or valine to generate a CasX variant protein of the disclosure.

Any permutation of the substitution, insertion and deletion embodiments described herein can be combined to generate a CasX variant protein of the disclosure. For example, a CasX variant protein can comprise at least one substitution and at least one deletion relative to a reference CasX protein sequence or a sequence of CasX 491 or CasX 515, at least one substitution and at least one insertion relative to a reference CasX protein sequence or a sequence of CasX 491 or CasX 515, at least one insertion and at least one deletion relative to a reference CasX protein sequence or a sequence of CasX 491 or CasX 515, or at least one substitution, one insertion and one deletion relative to a reference CasX protein sequence or a sequence of CasX 491 or CasX 515.

In some embodiments, the CasX variant protein comprises between 400 and 2000 amino acids, between 500 and 1500 amino acids, between 700 and 1200 amino acids, between 800 and 1100 amino acids, or between 900 and 1000 amino acids.

In some embodiments, a CasX variant protein comprises a sequence of SEQ ID NOS: 49-160, 40208-40369, or 40828-40912 as set forth in Table 3. In some embodiments, a CasX variant protein consists of a sequence of SEQ ID NOS: 49-160, 40208-40369, or 40828-40912 as set forth in Table 3. In other embodiments, a CasX variant protein comprises a sequence at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 86% identical, at least 87% identical, at least 88% identical, at least 89% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical to a sequence of SEQ ID NOS: 49-160, 40208-40369, or 40828-40912 as set forth in Table 3. In some embodiments, a CasX variant protein comprises or consists of a sequence of SEQ ID NOS: 49-160, 40208-40369, or 40828-40912 as set forth in Table 3. In other embodiments, a CasX variant protein comprises a sequence at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 86% identical, at least 87% identical, at least 88% identical, at least 89% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical to a sequence of SEQ ID NOS: 85-160, 40208-40369, or 40828-40912. In some embodiments, a CasX variant protein comprises or consists of a sequence of SEQ ID NOS: 85-160, 40208-40369, or 40828-40912.

c. CasX Variant Proteins with Domains from Multiple Source Proteins

In certain embodiments, the disclosure provides a chimeric CasX protein for use in the AAV systems comprising protein domains from two or more different CasX proteins, such as two or more reference CasX proteins, or two or more CasX variant protein sequences as described herein. As used herein, a “chimeric CasX protein” refers to a CasX containing at least two domains isolated or derived from different sources, such as two naturally occurring proteins, which may, in some embodiments, be isolated from different species. For example, in some embodiments, a chimeric CasX protein comprises a first domain from a first CasX protein and a second domain from a second, different CasX protein. In some embodiments, the first domain can be selected from the group consisting of the NTSB, TSL, Helical I, Helical IL, OBD and RuvC domains. In some embodiments, the second domain is selected from the group consisting of the NTSB, TSL, Helical I, Helical II, OBD and RuvC domains with the second domain being different from the foregoing first domain. For example, a chimeric CasX protein may comprise an NTSB, TSL, Helical I, Helical II, OBD domains from a CasX protein of SEQ ID NO: 2, and a RuvC domain from a CasX protein of SEQ ID NO: 1, or vice versa. As a further example, a chimeric CasX protein may comprise an NTSB, TSL, Helical II, OBD and RuvC domain from CasX protein of SEQ ID NO: 2, and a Helical I domain from a CasX protein of SEQ ID NO: 1, or vice versa. Thus, in certain embodiments, a chimeric CasX protein may comprise an NTSB, TSL, Helical II, OBD and RuvC domain from a first CasX protein, and a Helical I domain from a second CasX protein. In some embodiments of the chimeric CasX proteins, the domains of the first CasX protein are derived from the sequences of SEQ ID NO: 1, SEQ ID NO: 2 or SEQ ID NO: 3, and the domains of the second CasX protein are derived from the sequences of SEQ ID NO: 1, SEQ ID NO: 2 or SEQ ID NO: 3, and the first and second CasX proteins are not the same. In some embodiments, domains of the first CasX protein comprise sequences derived from SEQ ID NO: 1 and domains of the second CasX protein comprise sequences derived from SEQ ID NO: 2 In some embodiments, domains of the first CasX protein comprise sequences derived from SEQ ID NO: 1 and domains of the second CasX protein comprise sequences derived from SEQ ID NO: 3. In some embodiments, domains of the first CasX protein comprise sequences derived from SEQ ID NO: 2 and domains of the second CasX protein comprise sequences derived from SEQ ID NO: 3.

In some embodiments, a CasX variant protein comprises at least one chimeric domain comprising a first part from a first CasX protein and a second part from a second, different CasX protein. As used herein, a “chimeric domain” refers to a single domain containing at least two parts isolated or derived from different sources, such as two naturally occurring proteins or portions of domains from two reference CasX proteins. The at least one chimeric domain can be any of the NTSB, TSL, Helical I, Helical IL, OBD or RuvC domains as described herein. In some embodiments, the first portion of a CasX domain comprises a sequence of SEQ ID NO: 1 and the second portion of a CasX domain comprises a sequence of SEQ ID NO: 2. In some embodiments, the first portion of the CasX domain comprises a sequence of SEQ ID NO: 1 and the second portion of the CasX domain comprises a sequence of SEQ ID NO: 3. In some embodiments, the first portion of the CasX domain comprises a sequence of SEQ ID NO: 2 and the second portion of the CasX domain comprises a sequence of SEQ ID NO: 3. In some embodiments, the at least one chimeric domain comprises a chimeric RuvC domain. As an example of the foregoing, the chimeric RuvC domain comprises amino acids 661 to 824 of SEQ ID NO: 1 and amino acids 922 to 978 of SEQ ID NO: 2. As an alternative example of the foregoing, a chimeric RuvC domain comprises amino acids 648 to 812 of SEQ ID NO: 2 and amino acids 935 to 986 of SEQ ID NO: 1. In some embodiments, a CasX protein comprises a first domain from a first CasX protein and a second domain from a second CasX protein, and at least one chimeric domain comprising at least two parts isolated from different CasX proteins using the approach of the embodiments described in this paragraph.

In some embodiments, a CasX variant protein for use in the AAV systems comprises a sequence set forth in Table 3. In other embodiments, a CasX variant protein comprises a sequence at least 60% identical, at least 65% identical, at least 70a identical, at least 75% identical, at least 80% identical, at least 81% identical, at least 82% identical, at least 83% identical, at least 84% identical, at least 85% identical, at least 86% identical, at least 86% identical, at least 87% identical, at least 88% identical, at least 89% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical to a sequence selected from the group consisting of the sequences as set forth in Table 3.

TABLE 3
CasX Variant Sequences

| SEQ | | |
|---|---|---|
| ID NO | Variant | Description of Variant |
| 49 | ND | TSL, Helical I, Helical II, OBD and RuvC domains from SEQ ID NO: 2 and an NTSB domain |
| | | from SEQ ID NO: 1 |
| 50 | ND | NTSB, Helical I, Helical II, OBD and RuvC domains from SEQ ID NO: 2 and a TSL domain |
| | | from SEQ ID NO: 1. |
| 51 | ND | TSL, Helical I, Helical II, OBD and RuvC domains from SEQ ID NO: 1 and an NTSB domain |
| | | from SEQ ID NO: 2 |
| 52 | ND | NTSB, Helical I, Helical II, OBD and RuvC domains from SEQ ID NO: 1 and an TSL domain |
| | | from SEQ ID NO: 2. |
| 53 | ND | NTSB, TSL, Helical I, Helical II and OBD domains SEQ ID NO: 2 and an exogenous RuvC |
| | | domain or a portion thereof from a second CasX protein. |
| 54 | ND | No description |
| 55 | ND | NTSB, TSL, Helical II, OBD and RuvC domains from SEQ ID NO: 2 and a Helical I domain |
| | | from SEQ ID NO: 1 |
| 56 | ND | NTSB, TSL, Helical I, OBD and RuvC domains from SEQ ID NO: 2 and a Helical II domain |
| | | from SEQ ID NO: 1 |
| 57 | ND | NTSB, TSL, Helical I, Helical II and RuvC domains from a first CasX protein and an |
| | | exogenous OBD or a part thereof from a second CasX protein |
| 58 | ND | No description |
| 59 | ND | No description |
| 60 | ND | substitution of L379R, a substitution of C477K, a substitution of A708K, a deletion of P at |
| | | position 793 and a substitution of T620P of SEQ ID NO: 2 |
| 61 | ND | substitution of M771A of SEQ ID NO: 2. |
| 62 | ND | substitution of L379R, a substitution of A708K, a deletion of P at position 793 and a |
| | | substitution of D732N of SEQ ID NO: 2. |
| 63 | ND | substitution of W782Q of SEQ ID NO: 2. |
| 64 | ND | substitution of M771Q of SEQ ID NO: 2 |
| 65 | ND | substitution of R458I and a substitution of A739V of SEQ ID NO: 2. |
| 66 | ND | L379R, a substitution of A708K, a deletion of P at position 793 and a substitution of M771N |
| | | of SEQ ID NO: 2 |
| 67 | ND | substitution of L379R, a substitution of A708K, a deletion of P at position 793 and a |
| | | substitution of A739T of SEQ ID NO: 2 |
| 68 | ND | substitution of L379R, a substitution of C477K, a substitution of A708K, a deletion of P at |
| | | position 793 and a substitution of D489S of SEQ ID NO: 2. |
| 69 | ND | substitution of L379R, a substitution of C477K, a substitution of A708K, a deletion of P at |
| | | position 793 and a substitution of D732N of SEQ ID NO: 2. |
| 70 | ND | substitution of V711K of SEQ ID NO: 2. |
| 71 | ND | substitution of L379R, a substitution of C477K, a substitution of A708K, a deletion of P at |
| | | position 793 and a substitution of Y797L of SEQ ID NO: 2. |
| 72 | 119 | ND |
| 73 | ND | substitution of L379R, a substitution of C477K, a substitution of A708K, a deletion of P at |
| | | position 793 and a substitution of M771N of SEQ ID NO: 2. |
| 74 | ND | substitution of A708K, a deletion of P at position 793 and a substitution of E386S of SEQ ID |
| | | NO: 2. |
| 75 | ND | substitution of L379R, a substitution of C477K, a substitution of A708K and a deletion of P at |
| | | position 793 of SEQ ID NO: 2. |
| 76 | ND | substitution of L792D of SEQ ID NO: 2. |
| 77 | ND | substitution of G791F of SEQ ID NO: 2. |
| 78 | ND | substitution of A708K, a deletion of P at position 793 and a substitution of A739V of SEQ ID |
| | | NO: 2. |
| 79 | ND | substitution of L379R, a substitution of A708K, a deletion of P at position 793 and a |
| | | substitution of A739V of SEQ ID NO: 2. |
| 80 | ND | substitution of C477K, a substitution of A708K and a deletion of P at position 793 of SEQ ID |
| | | NO: 2. |
| 81 | ND | substitution of L249I and a substitution of M771N of SEQ ID NO: 2. |
| 82 | ND | substitution of V747K of SEQ ID NO: 2. |
| 83 | ND | substitution of L379R, a substitution of C477K, a substitution of A708K, a deletion of P at |
| | | position 793 and a substitution of M779N of SEQ ID NO: 2. |
| 84 | ND | L379R, F755M |
| 85 | 429 | ND |
| 86 | 430 | ND |
| 87 | 431 | ND |
| 88 | 432 | ND |
| 89 | 433 | ND |
| 90 | 434 | ND |
| 91 | 435 | ND |
| 92 | 436 | ND |
| 93 | 437 | ND |
| 94 | 438 | ND |
| 95 | 439 | ND |
| 96 | 440 | ND |
| 97 | 441 | ND |
| 98 | 442 | ND |
| 99 | 443 | ND |
| 100 | 444 | ND |
| 101 | 445 | ND |
| 102 | 446 | ND |
| 103 | 447 | ND |
| 104 | 448 | ND |
| 105 | 449 | ND |
| 106 | 450 | ND |
| 107 | 451 | ND |
| 108 | 452 | ND |
| 109 | 453 | ND |
| 110 | 454 | ND |
| 111 | 455 | ND |
| 112 | 456 | ND |
| 113 | 457 | ND |
| 114 | 458 | ND |
| 115 | 459 | ND |
| 116 | 460 | ND |
| 117 | 278 | ND |
| 118 | 279 | ND |
| 119 | 280 | ND |
| 120 | 285 | ND |
| 121 | 286 | ND |
| 122 | 287 | ND |
| 123 | 288 | ND |
| 124 | 290 | ND |
| 125 | 291 | ND |
| 126 | 293 | ND |
| 127 | 300 | ND |
| 128 | 492 | ND |
| 129 | 493 | ND |
| 130 | 387 | ND |
| 131 | 395 | ND |
| 132 | 485 | ND |
| 133 | 486 | ND |
| 134 | 487 | ND |
| 135 | 488 | ND |
| 136 | 489 | ND |
| 137 | 490 | ND |
| 138 | 491 | ND |
| 139 | 494 | ND |
| 140 | 328 | ND |
| 141 | 388 | ND |
| 142 | 389 | ND |
| 143 | 390 | ND |
| 144 | 514 | ND |
| 145 | 515 | ND |
| 146 | 516 | ND |
| 147 | 517 | ND |
| 148 | 518 | ND |
| 149 | 519 | ND |
| 150 | 520 | ND |
| 151 | 522 | ND |
| 152 | 523 | ND |
| 153 | 524 | ND |
| 154 | 525 | ND |
| 155 | 526 | ND |
| 156 | 527 | ND |
| 157 | 528 | ND |
| 158 | 529 | ND |
| 159 | 530 | ND |
| 160 | 531 | ND |
| 40208 | 532 | ND |
| 40209 | 533 | ND |
| 40210 | 534 | ND |
| 40211 | 535 | ND |
| 40212 | 536 | ND |
| 40213 | 537 | ND |
| 40214 | 538 | ND |
| 40215 | 539 | ND |
| 40216 | 540 | ND |
| 40217 | 541 | ND |
| 40218 | 542 | ND |
| 40219 | 543 | ND |
| 40220 | 544 | ND |
| 40221 | 545 | ND |
| 40222 | 546 | ND |
| 40223 | 547 | ND |
| 40224 | 548 | ND |
| 40225 | 550 | ND |
| 40226 | 551 | ND |
| 40227 | 552 | ND |
| 40228 | 553 | ND |
| 40229 | 554 | ND |
| 40230 | 555 | ND |
| 40231 | 556 | ND |
| 40232 | 557 | ND |
| 40233 | 558 | ND |
| 40234 | 559 | ND |
| 40235 | 560 | ND |
| 40236 | 561 | ND |
| 40237 | 562 | ND |
| 40238 | 563 | ND |
| 40239 | 564 | ND |
| 40240 | 565 | ND |
| 40241 | 566 | ND |
| 40242 | 567 | ND |
| 40243 | 568 | ND |
| 40244 | 569 | ND |
| 40246 | 570 | ND |
| 40247 | 571 | ND |
| 40248 | 572 | ND |
| 40249 | 573 | ND |
| 40250 | 574 | ND |
| 40251 | 575 | ND |
| 40252 | 576 | ND |
| 40253 | 577 | ND |
| 40254 | 578 | ND |
| 40255 | 579 | ND |
| 40256 | 580 | ND |
| 40257 | 581 | ND |
| 40258 | 582 | ND |
| 40259 | 583 | ND |
| 40260 | 584 | ND |
| 40261 | 585 | ND |
| 40262 | 586 | ND |
| 40263 | 587 | ND |
| 40264 | 588 | ND |
| 40265 | 589 | ND |
| 40266 | 590 | ND |
| 40267 | 591 | ND |
| 40268 | 592 | ND |
| 40269 | 593 | ND |
| 40270 | 594 | ND |
| 40271 | 595 | ND |
| 40272 | 596 | ND |
| 40273 | 597 | ND |
| 40274 | 598 | ND |
| 40275 | 599 | ND |
| 40276 | 600 | ND |
| 40277 | 601 | ND |
| 40278 | 602 | ND |
| 40279 | 603 | ND |
| 40280 | 604 | ND |
| 40281 | 605 | ND |
| 40282 | 606 | ND |
| 40283 | 607 | ND |
| 40284 | 608 | ND |
| 40285 | 609 | ND |
| 40286 | 610 | ND |
| 40287 | 611 | ND |
| 40288 | 612 | ND |
| 40289 | 613 | ND |
| 40290 | 614 | ND |
| 40291 | 615 | ND |
| 40292 | 616 | ND |
| 40293 | 617 | ND |
| 40294 | 618 | ND |
| 40295 | 619 | ND |
| 40296 | 620 | ND |
| 40297 | 621 | ND |
| 40298 | 622 | ND |
| 40299 | 623 | ND |
| 40300 | 624 | ND |
| 40301 | 625 | ND |
| 40302 | 626 | ND |
| 40303 | 627 | ND |
| 40304 | 628 | ND |
| 40305 | 629 | ND |
| 40306 | 630 | ND |
| 40307 | 631 | ND |
| 40308 | 632 | ND |
| 40309 | 633 | ND |
| 40310 | 634 | ND |
| 40311 | 635 | ND |
| 40312 | 636 | ND |
| 40313 | 637 | ND |
| 40314 | 638 | ND |
| 40315 | 639 | ND |
| 40316 | 640 | ND |
| 40317 | 641 | ND |
| 40318 | 642 | ND |
| 40319 | 643 | ND |
| 40320 | 644 | ND |
| 40321 | 645 | ND |
| 40322 | 646 | ND |
| 40323 | 647 | ND |
| 40324 | 648 | ND |
| 40325 | 649 | ND |
| 40326 | 650 | ND |
| 40327 | 651 | ND |
| 40328 | 652 | ND |
| 40329 | 653 | ND |
| 40330 | 654 | ND |
| 40331 | 655 | ND |
| 40332 | 656 | ND |
| 40333 | 657 | ND |
| 40334 | 658 | ND |
| 40335 | 659 | ND |
| 40336 | 660 | ND |
| 40337 | 661 | ND |
| 40338 | 662 | ND |
| 40339 | 663 | ND |
| 40340 | 664 | ND |
| 40341 | 665 | ND |
| 40342 | 666 | ND |
| 40343 | 667 | ND |
| 40344 | 668 | ND |
| 40345 | 669 | ND |
| 40346 | 671 | ND |
| 40347 | 672 | ND |
| 40348 | 673 | ND |
| 40349 | 674 | ND |
| 40350 | 675 | ND |
| 40351 | 676 | ND |
| 40352 | 677 | ND |
| 40353 | 678 | ND |
| 40354 | 679 | ND |
| 40355 | 680 | ND |
| 40356 | 681 | ND |
| 40357 | 682 | ND |
| 40358 | 683 | ND |
| 40359 | 684 | ND |
| 40360 | 685 | ND |
| 40361 | 686 | ND |
| 40362 | 687 | ND |
| 40363 | 688 | ND |
| 40364 | 689 | ND |
| 40365 | 690 | ND |
| 40366 | 691 | ND |
| 40367 | 692 | ND |
| 40368 | 693 | ND |
| 40369 | 694 | ND |
| 40828 | 701 | ND |
| 40829 | 702 | ND |
| 40830 | 703 | ND |
| 40831 | 704 | ND |
| 40832 | 705 | ND |
| 40833 | 706 | ND |
| 40834 | 707 | ND |
| 40835 | 708 | ND |
| 40836 | 709 | ND |
| 40837 | 710 | ND |
| 40838 | 711 | ND |
| 40839 | 712 | ND |
| 40840 | 713 | ND |
| 40841 | 714 | ND |
| 40842 | 715 | ND |
| 40843 | 716 | ND |
| 40844 | 717 | ND |
| 40845 | 718 | ND |
| 40846 | 719 | ND |
| 40847 | 720 | ND |
| 40848 | 721 | ND |
| 40849 | 722 | ND |
| 40850 | 723 | ND |
| 40851 | 724 | ND |
| 40852 | 725 | ND |
| 40853 | 726 | ND |
| 40854 | 727 | ND |
| 40855 | 728 | ND |
| 40856 | 729 | ND |
| 40857 | 730 | ND |
| 40858 | 731 | ND |
| 40859 | 732 | ND |
| 40860 | 733 | ND |
| 40861 | 734 | ND |
| 40862 | 735 | ND |
| 40863 | 736 | ND |
| 40864 | 737 | ND |
| 40865 | 738 | ND |
| 40866 | 739 | ND |
| 40867 | 740 | ND |
| 40868 | 741 | ND |
| 40869 | 742 | ND |
| 40870 | 743 | ND |
| 40871 | 744 | ND |
| 40872 | 745 | ND |
| 40873 | 746 | ND |
| 40874 | 747 | ND |
| 40875 | 748 | ND |
| 40876 | 749 | ND |
| 40877 | 750 | ND |
| 40878 | 751 | ND |
| 40879 | 752 | ND |
| 40880 | 753 | ND |
| 40881 | 754 | ND |
| 40882 | 755 | ND |
| 40883 | 756 | ND |
| 40884 | 757 | ND |
| 40885 | 758 | ND |
| 40886 | 759 | ND |
| 40887 | 760 | ND |
| 40888 | 761 | ND |
| 40889 | 762 | ND |
| 40890 | 763 | ND |
| 40891 | 764 | ND |
| 40892 | 765 | ND |
| 40893 | 766 | ND |
| 40894 | 767 | ND |
| 40895 | 768 | ND |
| 40896 | 769 | ND |
| 40897 | 770 | ND |
| 40898 | 777 | ND |
| 40899 | 778 | ND |
| 40900 | 779 | ND |
| 40901 | 780 | ND |
| 40902 | 781 | ND |
| 40903 | 782 | ND |
| 40904 | 783 | ND |
| 40905 | 784 | ND |
| 40906 | 785 | ND |
| 40907 | 786 | ND |
| 40908 | 787 | ND |
| 40909 | 788 | ND |
| 40910 | 789 | ND |
| 40911 | 790 | ND |
| 40912 | 791 | ND |

d. Protein Affinity for the gRNA

In some embodiments, a CasX variant protein for use in the AAV systems of the disclosure has improved affinity for the gRNA relative to a reference CasX protein, leading to the formation of the ribonucleoprotein complex. Increased affinity of the CasX variant protein for the gRNA may, for example, result in a lower K_{d} for the generation of a RNP complex, which can, in some cases, result in a more stable ribonucleoprotein complex formation. In some embodiments, increased affinity of the CasX variant protein for the gRNA results in increased stability of the ribonucleoprotein complex when delivered to human cells. This increased stability can affect the function and utility of the complex in the cells of a subject, as well as result in improved pharmacokinetic properties in blood, when delivered to a subject. In some embodiments, increased affinity of the CasX variant protein, and the resulting increased stability of the ribonucleoprotein complex, allows for a lower dose of the CasX variant protein to be delivered to the subject or cells while still having the desired activity, for example in vivo or in vitro gene editing. In some embodiments, a higher affinity (tighter binding) of a CasX variant protein to a gRNA allows for a greater amount of editing events when both the CasX variant protein and the gRNA remain in an RNP complex. Increased editing events can be assessed using editing assays such as the EGFP disruption assay described herein.

In some embodiments, the K_{d} of a CasX variant protein for a gRNA is increased relative to a reference CasX protein by a factor of at least about 1.1, at least about 1.2, at least about 1.3, at least about 1.4, at least about 1.5, at least about 1.6, at least about 1.7, at least about 1.8, at least about 1.9, at least about 2, at least about 3, at least about 4, at least about 5, at least about 6, at least about 7, at least about 8, at least about 9, at least about 10, at least about 15, at least about 20, at least about 25, at least about 30, at least about 35, at least about 40, at least about 45, at least about 50, at least about 60, at least about 70, at least about 80, at least about 90, or at least about 100. In some embodiments, the CasX variant has about 1.1 to about 10-fold increased binding affinity to the gRNA compared to the reference CasX protein of SEQ ID NO: 2.

In some embodiments, increased affinity of the CasX variant protein for the gRNA results in increased stability of the ribonucleoprotein complex when delivered to mammalian cells, including in vivo delivery to a subject. This increased stability can affect the function and utility of the complex in the cells of a subject, as well as result in improved pharmacokinetic properties in blood, when delivered to a subject. In some embodiments, increased affinity of the CasX variant protein, and the resulting increased stability of the ribonucleoprotein complex, allows for a lower dose of the CasX variant protein to be delivered to the subject or cells while still having the desired activity; for example in vivo or in vitro gene editing. The increased ability to form RNP and keep them in stable form can be assessed using assays such as the in vitro cleavage assays described in the Examples herein. In some embodiments, RNP comprising the CasX variants of the disclosure are able to achieve a k_{cleave} rate when complexed as an RNP that is at last 2-fold, at least 5-fold, or at least 10-fold higher compared to RNP comprising a reference CasX of SEQ ID NOS: 1-3.

In some embodiments, a higher affinity (tighter binding) of a CasX variant protein to a gRNA allows for a greater amount of editing events when both the CasX variant protein and the gRNA remain in an RNP complex. Increased editing events can be assessed using editing assays such as the assays described herein.

Without wishing to be bound by theory, in some embodiments amino acid changes in the Helical I domain can increase the binding affinity of the CasX variant protein with the gRNA targeting sequence, while changes in the Helical II domain can increase the binding affinity of the CasX variant protein with the gRNA scaffold stem loop, and changes in the oligonucleotide binding domain (OBD) increase the binding affinity of the CasX variant protein with the gRNA triplex.

Methods of measuring CasX protein binding affinity for a gRNA include in vitro methods using purified CasX protein and gRNA. The binding affinity for reference CasX and variant proteins can be measured by fluorescence polarization if the gRNA or CasX protein is tagged with a fluorophore. Alternatively, or in addition, binding affinity can be measured by biolayer interferometry, electrophoretic mobility shift assays (EMSAs), or filter binding. Additional standard techniques to quantify absolute affinities of RNA binding proteins such as the reference CasX and variant proteins of the disclosure for specific gRNAs such as reference gRNAs and variants thereof include, but are not limited to, isothermal calorimetry (ITC), and surface plasmon resonance (SPR), as well as the methods of the Examples.

e. Affinity for Target Nucleic Acid

In some embodiments, a CasX variant protein for use in the AAV systems of the disclosure has improved binding affinity for a target nucleic acid sequence relative to the affinity of a reference CasX protein for a target nucleic acid sequence. CasX variants with higher affinity for their target nucleic acid may, in some embodiments, cleave the target nucleic acid sequence more rapidly than a reference CasX protein that does not have increased affinity for the target nucleic acid. In some embodiments, the improved affinity for the target nucleic acid sequence comprises improved affinity for the target nucleic acid sequence, improved binding affinity to a wider spectrum of PAM sequences, an improved ability to search DNA for the target nucleic acid sequence, or any combinations thereof. Without wishing to be bound by theory, it is thought that CRISPR/Cas system proteins such as CasX may find their target nucleic acid sequences by one-dimension diffusion along a DNA molecule. The process is thought to include (1) binding of the ribonucleoprotein to the DNA molecule followed by (2) stalling at the target nucleic acid sequence, either of which may be, in some embodiments, affected by improved affinity of CasX proteins for a target nucleic acid sequence, thereby improving function of the CasX variant protein compared to a reference CasX protein.

In some embodiments, a CasX variant protein for use in the AAV systems has improved binding affinity for the non-target strand of the target nucleic acid. As used herein, the term “non-target strand” refers to the strand of the DNA target nucleic acid sequence that does not form Watson and Crick base pairs with the targeting sequence in the gRNA, and is complementary to the target strand. In some embodiments, the CasX variant protein has about 1.1 to about 100-fold increased binding affinity to the non-target stand of the target nucleic acid compared to the reference protein of SEQ ID NO: 1, SEQ ID NO: 2, or SEQ ID NO: 3, or to the CasX variants 119 (SEQ ID NO: 72) and CasX 491 (SEQ ID NO:138).

Methods of measuring CasX protein (such as reference or variant) affinity for a target nucleic acid molecule may include electrophoretic mobility shift assays (EMSAs), filter binding, isothermal calorimetry (ITC), and surface plasmon resonance (SPR), fluorescence polarization and biolayer interferometry (BLI). Further methods of measuring CasX protein affinity for a target include in vitro biochemical assays that measure DNA cleavage events over time.

In some embodiments, the CasX variant protein for use in the AAV systems is catalytically dead (dCasX). In some embodiments, the disclosure provides RNP comprising a catalytically-dead CasX protein that retains the ability to bind target DNA. An exemplary catalytically-dead CasX variant protein comprises one or more mutations in the active site of the RuvC domain of the CasX protein. In some embodiments, a catalytically-dead CasX variant protein comprises substitutions at residues 672, 769 and/or 935 of SEQ ID NO: 1. In some embodiments, a catalytically-dead CasX variant protein comprises substitutions of D672A, E769A and/or D935A in the reference CasX protein of SEQ ID NO: 1. In some embodiments, a catalytically-dead CasX protein comprises substitutions at amino acids 659, 765 and/or 922 of SEQ ID NO: 2. In some embodiments, a catalytically-dead CasX protein comprises D659A, E756A and/or D922A substitutions in a reference CasX protein of SEQ ID NO: 2. In further embodiments, a catalytically-dead reference CasX protein comprises deletions of all or part of the RuvC domain of the reference CasX protein. Exemplary dCasX sequences are provided as SEQ ID NOS: 40808-40827, 41006-41009 in Table 7.

In some embodiments, improved affinity for DNA of a CasX variant protein also improves the function of catalytically inactive versions of the CasX variant protein. In some embodiments, the catalytically inactive version of the CasX variant protein comprises one or mutations in the DED motif in the RuvC. Catalytically dead CasX variant proteins can, in some embodiments, be used for base editing or epigenetic modifications. With a higher affinity for DNA, in some embodiments, catalytically-dead CasX variant proteins can, relative to catalytically active CasX, find their target DNA faster, remain bound to target DNA for longer periods of time, bind target DNA in a more stable fashion, or a combination thereof, thereby improving the function of the catalytically-dead CasX variant protein.

f. Improved Specificity for a Target Site

In some embodiments, a CasX variant protein for use in the AAV systems has improved specificity for a target nucleic acid sequence relative to a reference CasX protein. As used herein, “specificity,” interchangeably referred to as “target specificity,” refers to the degree to which a CRISPR/Cas system ribonucleoprotein complex cleaves off-target sequences that are similar, but not identical to the target nucleic acid sequence; e.g., a CasX variant RNP with a higher degree of specificity would exhibit reduced off-target cleavage of sequences relative to a reference CasX protein. The specificity, and the reduction of potentially deleterious off-target effects, of CRISPR/Cas system proteins can be vitally important in order to achieve an acceptable therapeutic index for use in mammalian subjects.

Without wishing to be bound by theory, it is possible that amino acid changes in the Helical I and II domains that increase the specificity of the CasX variant protein for the target nucleic acid strand can increase the specificity of the CasX variant protein for the target nucleic acid sequence overall. In some embodiments, amino acid changes that increase specificity of CasX variant proteins for target nucleic acid sequence may also result in decreased affinity of CasX variant proteins for DNA.

Methods of testing CasX protein (such as variant or reference) target specificity may include guide and Circularization for In vitro Reporting of Cleavage Effects by Sequencing (CIRCLE-seq), or similar methods. In brief, in CIRCLE-seq techniques, genomic DNA is sheared and circularized by ligation of stem-loop adapters, which are nicked in the stem-loop regions to expose 4 nucleotide palindromic overhangs. This is followed by intramolecular ligation and degradation of remaining linear DNA. Circular DNA molecules containing a CasX cleavage site are subsequently linearized with CasX, and adapter adapters are ligated to the exposed ends followed by high-throughput sequencing to generate paired end reads that contain information about the off-target site. Additional assays that can be used to detect off-target events, and therefore CasX protein specificity include assays used to detect and quantify indels (insertions and deletions) formed at those selected off-target sites such as mismatch-detection nuclease assays and next generation sequencing (NGS). Exemplary mismatch-detection assays include nuclease assays, in which genomic DNA from cells treated with CasX and sgRNA is PCR amplified, denatured and rehybridized to form hetero-duplex DNA, containing one wild type strand and one strand with an indel. Mismatches are recognized and cleaved by mismatch detection nucleases, such as Surveyor nuclease or T7 endonuclease I.

g. Protospacer and PAM Sequences

Herein, the protospacer is defined as the DNA sequence complementary to the targeting sequence of the guide RNA and the DNA complementary to that sequence, referred to as the target strand and non-target strand, respectively. As used herein, the PAM is a nucleotide sequence proximal to the protospacer that, in conjunction with the targeting sequence of the gRNA, helps the orientation and positioning of the CasX for the potential cleavage of the protospacer strand(s).

PAM sequences may be degenerate, and specific RNP constructs may have different preferred and tolerated PAM sequences that support different efficiencies of cleavage. Following convention, unless stated otherwise, the disclosure refers to both the PAM and the protospacer sequence and their directionality according to the orientation of the non-target strand. This does not imply that the PAM sequence of the non-target strand, rather than the target strand, is determinative of cleavage or mechanistically involved in target recognition. For example, when reference is to a TTC PAM, it may in fact be the complementary GAA sequence that is required for target cleavage, or it may be some combination of nucleotides from both strands. In the case of the CasX proteins disclosed herein, the PAM is located 5′ of the protospacer with a single nucleotide separating the PAM from the first nucleotide of the protospacer. Thus, in the case of reference CasX, in which the canonical PAM is TTC, the PAM should be understood to mean a sequence following the formula 5′- . . . NNTTCN(protospacer)NNNNNN . . . 3′ where ‘N’ is any DNA nucleotide and ‘(protospacer)’ is a DNA sequence having identity with the targeting sequence of the guide RNA. In the case of a CasX variant with expanded PAM recognition, a TTC, CTC, GTC, or ATC PAM should be understood to mean a sequence following the formulae:

### 5′- . . . NNTTCN(protospacer)NNNNNN . . . 3′;

### 5′- . . . NNCTCN(protospacer)NNNNNN . . . 3′;

### 5′- . . . NNGTCN(protospacer)NNNNNN . . . 3′; or

5′- . . . NNATCN(protospacer)NNNNNN . . . 3′. Alternatively, a TC PAM should be understood to mean a sequence following the formula 5′- . . . NNNTCN(protospacer)NNNNNN . . . 3′.

Additionally, the CasX variant proteins of the disclosure have an enhanced ability to efficiently edit and/or bind target nucleic acid, when complexed with a gRNA as an RNP, utilizing a PAM TC motif, including PAM sequences selected from TTC, ATC, GTC, or CTC, (in a 5′ to 3′ orientation), compared to an RNP of a reference CasX protein and reference gRNA, or to an RNP of another CasX variant from which it was derived, such as CasX 491, and gRNA 174. In the foregoing, the PAM sequence is located at least 1 nucleotide 5′ to the non-target strand of the protospacer having identity with the targeting sequence of the gRNA in an assay system compared to the editing efficiency and/or binding of an RNP comprising a reference CasX protein and reference gRNA in a comparable assay system. In one embodiment, an RNP of a CasX variant and gRNA variant exhibits greater editing efficiency and/or binding of a target sequence in the target nucleic acid compared to an RNP comprising a reference CasX protein and a reference gRNA (or an RNP of another CasX variant from which it was derived, such as CasX 491, and gRNA 174) in a comparable assay system, wherein the PAM sequence of the target DNA is TTC. In another embodiment, an RNP of a CasX variant and gRNA variant exhibits greater editing efficiency and/or binding of a target sequence in the target nucleic acid compared to an RNP comprising a reference CasX protein and a reference gRNA (or an RNP of another CasX variant from which it was derived, such as CasX 491 and gRNA 174) in a comparable assay system, wherein the PAM sequence of the target DNA is ATC. In a particular embodiment of the foregoing, wherein the CasX variant exhibits enhanced editing with an ATC PAM, the CasX variant is 528 (SEQ ID NO: 157). In another embodiment, an RNP of a CasX variant and gRNA variant exhibits greater editing efficiency and/or binding of a target sequence in the target nucleic acid compared to an RNP comprising a reference CasX protein and a reference gRNA (or an RNP of another CasX variant from which it was derived, such as CasX 491, and gRNA 174) in a comparable assay system, wherein the PAM sequence of the target DNA is CTC. In another embodiment, an RNP of a CasX variant and gRNA variant exhibits greater editing efficiency and/or binding of a target sequence in the target nucleic acid compared to an RNP comprising a reference CasX protein and a reference gRNA (or an RNP of another CasX variant from which it was derived and gRNA 174) in a comparable assay system, wherein the PAM sequence of the target DNA is GTC. In the foregoing embodiments, the increased editing efficiency and/or binding affinity for the one or more PAM sequences is at least 1.5-fold, at least 2-fold, at least 4-fold, at least 10-fold, at least 20-fold, at least 30-fold, or at least 40-fold greater or more compared to the editing efficiency and/or binding affinity of an RNP of any one of the CasX proteins of SEQ ID NOS: 1-3 and the gRNA comprising a sequence of Table 1 for the PAM sequences. Exemplary assays demonstrating the improved editing are described herein, in the Examples. In some embodiments, a CasX protein can bind and/or modify (e.g., cleave, nick, methylate, demethylate, etc.) a target nucleic acid and/or a polypeptide associated with target nucleic acid (e.g., methylation or acetylation of a histone tail). In some embodiments, the CasX protein is catalytically-dead (dCasX) but retains the ability to bind a target nucleic acid.

h. Affinity for Target RNA

In some embodiments, variants of a reference CasX protein for use in the AAV systems of the disclosure have increased specificity for a target RNA, and increased the activity with respect to a target RNA when compared to the reference CasX protein. For example, CasX variant proteins can display increased binding affinity for target RNAs, or increased cleavage of target RNAs, when compared to reference CasX proteins. In some embodiments, a ribonucleoprotein complex comprising a CasX variant protein binds to a target RNA and/or cleaves the target RNA. In some embodiments, a CasX variant has at least about two-fold to about 10-fold increased binding affinity to the target RNA compared to the reference protein of SEQ ID NO: 1, SEQ ID NO: 2, or SEQ ID NO: 3.

i. CasX Variant Proteins with Domains from Multiple Source Proteins

In some embodiments, the disclosure provides AAV encoding a chimeric CasX variant protein comprising protein domains from two or more different CasX proteins, such as two or more naturally occurring CasX proteins, or two or more CasX variant protein sequences as described herein. As used herein, a “chimeric CasX protein” refers to a CasX containing at least two domains isolated or derived from different sources, such as two naturally occurring proteins, which may, in some embodiments, be isolated from different species. For example, in some embodiments, a chimeric CasX protein comprises a first domain from a first CasX protein and a second domain from a second, different CasX protein. In some embodiments, the first domain can be selected from the group consisting of the NTSB, TSL, helical I, helical II, OBD and RuvC domains. In some embodiments, the second domain is selected from the group consisting of the NTSB, TSL, helical I, helical II, OBD and RuvC domains with the second domain being different from the foregoing first domain. For example, a chimeric CasX protein may comprise an NTSB, TSL, helical I, helical IL, OBD domains from a CasX protein of SEQ ID NO: 2, and a RuvC domain from a CasX protein of SEQ ID NO: 1, or vice versa. As a further example, a chimeric CasX protein may comprise an NTSB, TSL, helical II, OBD and RuvC domain from CasX protein of SEQ ID NO: 2, and a helical I domain from a CasX protein of SEQ ID NO: 1, or vice versa. Thus, in certain embodiments, a chimeric CasX protein may comprise an NTSB, TSL, helical II, OBD and RuvC domain from a first CasX protein, and a helical I domain from a second CasX protein. In some embodiments of the chimeric CasX proteins, the domains of the first CasX protein are derived from the sequences of SEQ ID NO: 1, SEQ ID NO: 2 or SEQ ID NO: 3, and the domains of the second CasX protein are derived from the sequences of SEQ ID NO: 1, SEQ ID NO: 2 or SEQ ID NO: 3, and the first and second CasX proteins are not the same. In some embodiments, domains of the first CasX protein comprise sequences derived from SEQ ID NO: 1 and domains of the second CasX protein comprise sequences derived from SEQ ID NO: 2. In some embodiments, domains of the first CasX protein comprise sequences derived from SEQ ID NO: 1 and domains of the second CasX protein comprise sequences derived from SEQ ID NO: 3. In some embodiments, domains of the first CasX protein comprise sequences derived from SEQ ID NO: 2 and domains of the second CasX protein comprise sequences derived from SEQ ID NO: 3. As an example of the foregoing, the chimeric RuvC domain comprises amino acids 660 to 823 of SEQ ID NO: 1 and amino acids 921 to 978 of SEQ ID NO: 2. As an alternative example of the foregoing, a chimeric RuvC domain comprises amino acids 647 to 810 of SEQ ID NO: 2 and amino acids 934 to 986 of SEQ ID NO: 1. In some embodiments, the at least one chimeric domain comprises a chimeric helical I domain wherein the chimeric helical I domain comprises amino acids 56-99 of SEQ ID NO: 1 and amino acids 192-332 of SEQ ID NO: 2. In some embodiments, the chimeric CasX variant is further modified, including the CasX variants selected from the group consisting of the sequences of SEQ ID NO: 40959, SEQ ID NO: 40960, SEQ ID NO: 40968, SEQ ID NO: 40977, SEQ ID NO: 40969, SEQ ID NO: 40970, SEQ ID NO: 40971, SEQ ID NO: 40972, SEQ ID NO: 40973, SEQ ID NO: 40961, SEQ ID NO: 40978, SEQ ID NO: 40962, SEQ ID NO: 40979, SEQ ID NO: 40963, SEQ ID NO: 40980, SEQ ID NO: 40964, SEQ ID NO: 40981, SEQ ID NO: 40965, SEQ ID NO: 40982, SEQ ID NO: 40966, SEQ ID NO: 40983, SEQ ID NO: 40967, SEQ ID NO: 40974, SEQ ID NO: 40975, SEQ ID NO: 40976, SEQ ID NO: 40984, and SEQ ID NO: 40985. In some embodiments, the one or more additional modifications comprises an insertion, substitution or deletion as described herein.

In the case of split or non-contiguous domains such as helical I, RuvC and OBD, a portion of the non-contiguous domain can be replaced with the corresponding portion from any other source. For example, the helical I-I domain (sometimes referred to as helical I-a) in SEQ ID NO: 2 can be replaced with the corresponding helical I-I sequence from SEQ ID NO: 1, and the like. Domain sequences from reference CasX proteins, and their coordinates, are shown in Table 4. Representative examples of chimeric CasX proteins include the variants of CasX 472-483, 485-491 and 515, the sequences of which are set forth in Table 3.

TABLE 4
Domain coordinates in Reference CasX proteins

| | Coordinates in | Coordinates in |
|---|---|---|
| Domain Name | SEQ ID NO: 1 | SEQ ID NO: 2 |
| OBD-I | 1-55 | 1-57 |
| helical I-I | 56-99 | 58-101 |
| NTSB | 100-190 | 102-191 |
| helical I-II | 191-331 | 192-332 |
| helical II | 332-508 | 333-500 |
| OBD-II | 509-659 | 501-646 |
| RuvC-I | 660-823 | 647-810 |
| TSL | 824-933 | 811-920 |
| RuvC-II | 934-986 | 921-978 |

*OBD I and II, helical I-I and I-II, and RuvC I and II are also referred to herein as OBD a and b, helical I a and b, and RuvC a and b.

Exemplary domain sequences are provided in Table 5 below.

TABLE 5
Exemplary Domain Sequences
Deltaproteobacter sp. (reference CasX of SEQ ID NO: 1)

| SEQ | | |
|---|---|---|
| ID | Domain | Sequence |
| 40986 | OBD-I | EKRINKIRKKLSADNATKPVSRSGPMKTLLVRVMTDDLKKRLEKRRKKPEVMPQ |
| 40987 | helical I-I | VISNNAANNLRMLLDDYTKMKEAILQVYWQEFKDDHVGLMCKFA |
| 40988 | NTSB | QPASKKIDQNKLKPEMDEKGNLTTAGFACSQCGQPLFVYKLEQVSEKGKAYTNY |
| | | FGRCNVAEHEKLILLAQLKPEKDSDEAVTYSLGKFGQ |
| 40989 | helical I-II | RALDFYSIHVTKESTHPVKPLAQIAGNRYASGPVGKALSDACMGTIASFLSKYQD |
| | | IIIEHQKVVKGNQKRLESLRELAGKENLEYPSVTLPPQPHTKEGVDAYNEVIARVR |
| | | MWVNLNLWQKLKLSRDDAKPLLRLKGFPSF |
| 40990 | helical II | PVVERRENEVDWWNTINEVKKLIDAKRDMGRVFWSGVTAEKRNTILEGYNYLP |
| | | NENDHKKREGSLENPKKPAKRQFGDLLLYLEKKYAGDWGKVFDEAWERIDKKI |
| | | AGLTSHIEREEARNAEDAQSKAVLTDWLRAKASFVLERLKEMDEKEFYACEIQL |
| | | QKWYGDLRG NPFAVEAE |
| 40991 | OBD-II | NRVVDISGFSIGSDGHSIQYRNLLAWKYLENGKREFYLLMNYGKKGRIRFTDGTD |
| | | IKKSGKWQGLLYGGGKAKVIDLTFDPDDEQLIILPLAFGTRQGREFIWNDLLSLET |
| | | GLIKLANGRVIEKTIYNKKIGRDEPALFVALTFERREVVD |
| 40992 | RuvC-I | PSNIKPVNLIGVDRGENIPAVIALTDPEGCPLPEFKDSSGGPTDILRIGEGYKEKQR |
| | | AIQAAKEVEQRRAGGYSRKFASKSRNLADDMVRNSARDLFYHAVTHDAVLVFE |
| | | NLSRGFGRQGKRTFMTERQYTKMEDWLTAKLAYEGLTSKTYLSKTLAQYTSKT |
| | | C |
| 40993 | TSL | SNCGFTITTADYDGMLVRLKKTSDGWATTLNNKELKAEGQITYYNRYKRQTVE |
| | | KELSAELDRLSEESGNNDISKWTKGRRDEALFLLKKRFSHRPVQEQFVCLDCGHE |
| | | VH |
| 40994 | RuvC-II | ADEQAALNIARSWLFLN SNSTEFKSYKSGKQPFVGAWQAFYKRRLKEVWKPNA |

Planctomycetes sp. (Reference CasX of SEQ ID NO: 2)

| SEQ | | |
|---|---|---|
| ID | Domain | Sequence |
| 40995 | OBD-I | QEIKRINKIRRRLVKDSNTKKAGKTGPMKTLLVRVMTPDLRERLENLRKKPENIP |
| | | Q |
| 40996 | helical I-II | PISNTSRANLNKLLTDYTEMKKAILHVYWEEFQKDPVGLMSRVA |
| 40997 | NTSB | QPAPKNIDQRKLIPVKDGNERLTSSGFACSQCCQPLYVYKLEQVNDKGKPHTNYF |
| | | GRCNVSEHERLILLSPHKPEANDELVTYSLGKFGQ |
| 40998 | helical I-II | RALDFYSIHVTRESNHPVKPLEQIGGNSCASGPVGKALSDACMGAVASFLTKYQ |
| | | DIILEHQKVIKKNEKRLANLKDIASANGLAFPKITLPPQPHTKEGIEAYNNVVAQI |
| | | VIWVNLNLWQKLKIGRDEAKPLQRLKGFPSF |
| 40999 | helical II | PLVERQANEVDWWDMVCNVKKLINEKKEDGKVFWQNLAGYKRQEALLPYLSS |
| | | EEDRKKGKKFARYQFGDLLLHLEKKHGEDWGKVYDEAWERIDKKVEGLSKHIK |
| | | LEEERRSEDAQSKAALTDWLRAKASFVIEGLKEADKDEFCRCELKLQKWYGDLR |
| | | GKPFAIEAE |
| 41000 | OBD-II | NSILDISGFSKQYNCAFIWQKDGVKKLNLYLIINYFKGGKLRFKKIKPEAFEANRF |
| | | YTVINKKSGEIVPMEVNFNFDDPNLIILPLAFGKRQGREFIWNDLLSLETGSLK |
| | | LANGRVIEKTLYNRRTRQDEPALFVALTFERREVLD |
| 41001 | RuvC-I | SSNIKPMNLIGIDRGENIPAVIALTDPEGCPLSRFKDSLGNPTHILRIGESYKEKQRT |
| | | IQAAKEVEQRRAGGYSRKYASKAKNLADDMVRNTARDLLYYAVTQDAMLIFEN |
| | | LSRGFGRQGKRTFMAERQYTRMEDWLTAKLAYEGLPSKTYLSKTLAQYTSKTC |
| 41002 | TSL | SNCGFTITSADYDRVLEKLKKTATGWMTTINGKELKVEGQITYYNRYKRQNVVK |
| | | DLSVELDRLSEESVNNDISSWTKGRSGEALSLLKKRFSHRPVQEKFVCLNCGFET |
| | | H |
| 41003 | RuvC-II | ADEQAALNIARSWLFLRSQEYKKYQTNKTTGNTDKRAFVETWQSFYRKKLKEV |
| | | WKPAV |

A further exemplary helical II domain sequence is provided as SEQ ID NO: 41004, and a further exemplary RuvC a domain sequence is provided as SEQ ID NO: 41005.

In other embodiments, a CasX variant protein comprises a sequence of SEQ ID NOS: 49-160, 40208-40286, or 40828-40912 as set forth in Table 3, and further comprises one or more NLS disclosed herein at or near either the N-terminus, the C-terminus, or both. In other embodiments, a CasX variant protein comprises a sequence of SEQ ID NOS: 72-160, 40208-40286, or 40828-40912, and further comprises one or more NLS disclosed herein at or near either the N-terminus, the C-terminus, or both. In other embodiments, a CasX variant protein comprises a sequence of SEQ ID NOS: 144-160, 40208-40286, or 40828-40912, and further comprises one or more NLS disclosed herein at or near either the N-terminus, the C-terminus, or both. It will be understood that in some cases, the N-terminal methionine of the CasX variants of the Tables is removed from the expressed CasX variant during post-translational modification. The person of ordinary skill in the art will understand that an NLS near the N or C terminus of a protein can be within 1, 2, 3, 4, 5, 6, 7, 8, 9, 10, 11, 12, 13, 14, 15, 16, 17, 18, 20 or 20 amino acids of the N or C terminus.

j. CasX Variants Derived from Other CasX Variants

In further iterations of the generation of variant proteins, a variant protein can be utilized to generate additional CasX variants of the disclosure. For example, CasX 119 (SEQ ID NO: 72), CasX 491 (SEQ ID NO: 138), and CasX 515 (SEQ ID NO: 145) are exemplary variant proteins that are modified to generate additional CasX variants of the disclosure having improvements or additional properties relative to a reference CasX or CasX variants from which they were derived. CasX 119 contains a substitution of L379R, a substitution of A708K and a deletion of P at position 793 of SEQ ID NO: 2. CasX 491 contains NTSB and Helical 1B swap from SEQ ID NO: 1. CasX 515 was derived from CasX 491 by insertion of P at position 793 (relative to SEQ ID NO:2) and was used to create the CasX variants described in Example 21. For example, CasX 668 has an insertion of R at position 26 and a substitution of G223S relative to CasX 515. CasX 672 has substitutions of L169K and G223S relative to CasX 515. CasX 676 has substitutions of L169K and G223S and an insertion of R at position 26 relative to CasX 515.

Exemplary methods used to generate and evaluate CasX variants derived from other CasX variants are described in the Examples, which were created by introducing modifications to the encoding sequence resulting in amino acid substitutions, deletions, or insertions at one or more positions in one or more domains of the CasX variant. In particular, Example 21 describes the methods used to create variants of CasX 515 (SEQ ID NO: 145) that were then assayed to determine those positions in the sequence that, when modified by an amino acid insertion, deletion or substitution, resulted in an enrichment or improvement in the assays. For purposes of the disclosure, the sequences of the domains of CasX 515 are provided in Table 6 and include an OBD-I domain having the sequence of SEQ ID NO: 40995, an OBD-II domain having the sequence of SEQ ID NO: 41000, NTSB domain having the sequence of SEQ ID NO: 40988, a helical I-I domain having the sequence of SEQ ID NO: 40996, a helical I-II domain having the sequence of SEQ ID NO: 40989, a helical II domain having the sequence of SEQ ID NO: 41004, a RuvC-I domain having the sequence of SEQ ID NO: 41005, a RuvC-II domain having the sequence of SEQ ID NO: 41003, and a TSL domain having the sequence of SEQ ID NO: 41002. By the methods of the disclosure, individual positions in the domains of CasX 515 were modified, assayed, and the resulting positions and exemplary modifications leading to an enrichment or improvement that follow are provided, relative to their position in each domain or subdomain. In some cases, such positions are disclosed in Tables 30-33 of the Examples. In some embodiments, the disclosure provides CasX variants derived from CasX 515 comprising one or more modifications (i.e., an insertion, a deletion, or a substitution) at one or more amino acid positions in the NTSB domain relative to the NTSB domain sequence (SEQ ID NO: 40988) selected from the group consisting of P2, S4, Q9, E15, G20, G33, L41, Y51, F55, L68, A70, E75, K88, and G90, wherein the modification results in an improved characteristic relative to CasX 515. In a particular embodiment, the one or more modifications at one or more amino acid positions in the NTSB domain relative to the NTSB domain sequence (SEQ ID NO: 40988) are selected from the group consisting of \hat{ }G2, \hat{ }I4, \hat{ }L4, Q9P, E15S, G20D, [S30], G33T, L41A, Y51T, F55V, L68D, L68E, L68K, A70Y, A70S, E75A, E75D, E75P, K88Q, and G90Q (where “\hat{ }” represents and insertion and “[ ]” represents a deletion at that position). In some embodiments, the disclosure provides CasX variants derived from CasX 515 comprising one or more modifications at one or more amino acid positions in the helical I-II domain relative to the helical I-II domain sequence (SEQ ID NO: 40989) selected from the group consisting of 124, A25, Y29 G32, G44, S48, S51, Q54, 156, V63, S73, L74, K97, V100, M112, L116, G137, F138, and S140, wherein the modification results in an improved characteristic relative to CasX 515. In a particular embodiment, the one or more modifications at one or more amino acid positions in the helical I-II domain are selected from the group consisting of \hat{ }T24, \hat{ }C25, Y29F, G32Y, G32N, G32H, G32S, G32T, G32A, G32V, [G32], G32S, G32T, G44L, G44H, S48H, S48T, S51T, Q54H, I56T, V63T, S73H, L74Y, K97G, K97S, K97D, K97E, V100L, M112T, M112W, M112R, M112K, L116K, G137R, G137K, G137N, \hat{ }Q138, and S140Q. In some embodiments, the disclosure provides CasX variants derived from CasX 515 comprising one or more modifications at one or more amino acid positions in the helical II domain relative to the helical II domain sequence (SEQ ID NO: 41004) selected from the group consisting of L2, V3, E4, R5, Q6, A7, E9, V10, D11, W12, W13, D14, M15, V16, C17, N18, V19, K20, L22, I23, E25, K26, K31, Q35, L37, A38, K41, R42, Q43, E44, L46, K57, Y65, G68, L70, L71, L72, E75, G79, D81, W82, K84, V85, Y86, D87, I93, K95, K96, E98, L100, K102, I104, K105, E109, R110, D114, K118, A120, L121, W124, L125, R126, A127, A129, I133, E134, G135, L136, E138, D140, K141, D142, E143, F144, C145, C147, E148, L149, K150, L151, Q152, K153, L158, E166, and A167, wherein the modification results in an improved characteristic relative to CasX 515. In a particular embodiment, the one or more modifications at one or more amino acid positions in the helical II domain are selected from the group consisting of \hat{ }A2, \hat{ }H2, [L2]+[V3], V3E, V3Q, V3F, [V3], \hat{ }D3, V3P, E4P, [E4], E4D, E4L, E4R, R5N, Q6V, \hat{ }Q6, \hat{ }G7, \hat{ }H9, \hat{ }A9, VD10, \hat{ }T10, [V10], \hat{ }F10, \hat{ }D11, [D11], D11S, [W12], W12T, W12H, \hat{ }P12, \hat{ }Q13, \hat{ }G12, \hat{ }R13, W13P, W13D, \hat{ }D13, W13L, \hat{ }P14, \hat{ }D14, [D14]+[M15], [M15], \hat{ }T16, \hat{ }P17, N18I, V19N, V19H, K20D, L22D, I23S, E25C, E25P, \hat{ }G25, K26T, K27E, K31L, K31Y, Q35D, Q35P, \hat{ }S37, [L37]+[A38], K41L, \hat{ }R42, [Q43]+[E44], L46N, K57Q, Y65T, G68M, L70V, L71C, L72D, L72N, L72W, L72Y, E75F, E75L, E75Y, G79P, \hat{ }E79, \hat{ }T81, \hat{ }R81, \hat{ }W81, \hat{ }Y81, \hat{ }W82, \hat{ }Y82, W82G, W82R, K84D, K84H, K84P, K84T, V85L, V85A, \hat{ }L85, Y86C, D87G, D87M, D87P, I93C, K95T, K96R, E98G, L100A, K102H, I104T, I104S, I104Q, K105D, \hat{ }K109, E109L, R 110D, [R110], D114E, \hat{ }D114, K118P, A120R, L121T, W124L, L125C, R126D, A127E, A127L, A129T, A129K, I133E, \hat{ }C133, \hat{ }S134, \hat{ }G134, \hat{ }R135, G135P, L136K, L136D, L136S, L136H, [E138], D140R, \hat{ }D140, \hat{ }P141, \hat{ }D142, [E143]+[F144], \hat{ }Q143, F144K, [F144], [F144]+[C145], C145R, \hat{ }G145, C145K, C147D, \hat{ }V148, E148D, \hat{ }H149, L149R, K150R, L151H, Q152C, K153P, L158S, E166L, and \hat{ }F167. In some embodiments, the disclosure provides CasX variants derived from CasX 515 comprising one or more modifications at one or more amino acid positions in the RuvC-I domain relative to the RuvC-I domain sequence (SEQ ID NO: 41005) selected from the group consisting of 14, K5, P6, M7, N8, L9, V12, G49, K63, K80, N83, R90, M125, and L146, wherein the modification results in an improved characteristic relative to CasX 515. In a particular embodiment, the one or more modifications at one or more amino acid positions in the RuvC-I domain are selected from the group consisting of \hat{ }I4, \hat{ }S5, \hat{ }T6, \hat{ }N6, \hat{ }R7, \hat{ }K7, \hat{ }H8, \hat{ }S8, V12L, G49W, G49R, S51R, S51K, K62S, K62T, K62E, V65A, K80E, N83G, R90H, R90G, M125S, M125A, L137Y, \hat{ }P137, [L141], L141R, L141D, \hat{ }Q142, \hat{ }R143, \hat{ }N143, E144N, \hat{ }P146, L146F, P147A, K149Q, T150V, \hat{ }R152, \hat{ }H153, T155Q, \hat{ }H155, \hat{ }R155, \hat{ }L156, [L156], \hat{ }W156, \hat{ }A157, \hat{ }F157, A157S, Q158K, [Y159], T160Y, T160F, \hat{ }I161, S161P, T163P, \hat{ }N163, C164K, and C164M. In some embodiments, the disclosure provides CasX variants derived from CasX 515 comprising one or more modifications at one or more amino acid positions in the OBD-I domain relative to the OBD-I domain sequence (SEQ ID NO: 40995) selected from the group consisting of 14, K5, P6, M7, N8, L9, V12, G49, K63, K80, N83, R90, M125, and L146, wherein the modification results in an improved characteristic relative to CasX 515. In a particular embodiment, the one or more modifications at one or more amino acid positions in the OBD-I domain are selected from the group consisting of \hat{ }G3, I3G, I3E, \hat{ }G4, K4G, K4P, K4S, K4W, K4W, R5P, \hat{ }P5, \hat{ }G5, R5S, \hat{ }S5, R5A, R5P, R5G, R5L, I6A, I6L, \hat{ }G6, N7Q, N7L, N7S, K8G, K15F, D16W, \hat{ }F16, \hat{ }F18, \hat{ }P27, M28P, M28H, V33T, R34P, M36Y, R41P, L47P, \hat{ }P48, E52P, \hat{ }P55, [P55]+[Q56], Q56S, Q56P, \hat{ }D56, \hat{ }T56, and Q56P. In some embodiments, the disclosure provides CasX variants derived from CasX 515 comprising one or more modifications at one or more amino acid positions in the OBD-II domain relative to the OBD-II domain sequence (SEQ ID NO: 41000) selected from the group consisting of 14, K5, P6, M7, N8, L9, V12, G49, K63, K80, N83, R90, M125, and L146, wherein the modification results in an improved characteristic relative to CasX 515. In a particular embodiment, the one or more modifications at one or more amino acid positions in the OBD-I domain are selected from the group consisting of [S2], I3R, I3K, [I3]+[L4], [L4], K11T, \hat{ }P24, K37G, R42E, \hat{ }S53, \hat{ }R58, [K63], M70T, I82T, Q92L, Q92F, Q92V, Q92A, \hat{ }A93, K110Q, R115Q, L121T, \hat{ }A124, \hat{ }R141, \hat{ }D143, \hat{ }A143, \hat{ }W144, and \hat{ }A145. In some embodiments, the disclosure provides CasX variants derived from CasX 515 comprising one or more modifications at one or more amino acid positions in the TSL domain relative to the TSL domain sequence (SEQ ID NO: 41002) selected from the group consisting of S1, N2, C3, G4, F5, 17, K18, V58, S67, T76, G78, S80, G81, E82, S85, V96, and E98, wherein the modification results in an improved characteristic relative to CasX 515. In a particular embodiment, the one or more modifications at one or more amino acid positions in the OBD-I domain are selected from the group consisting of \hat{ }M1, [N2], \hat{ }V2, C3S, \hat{ }G4, \hat{ }W4, F5P, \hat{ }W7, K18G, V58D, \hat{ }A67, T76E, T76D, T76N, G78D, [S80], [G81], \hat{ }E82, \hat{ }N82, S85I, V96C, V96T, and E98D. It will be understood that combinations of any of the same foregoing modifications of the paragraph can similarly be introduced into the CasX variants of the disclosure, resulting in a CasX variant with improved characteristics. For example, in one embodiment, the disclosure provides CasX variant 535 (SEQ ID NO: 40211), which has a single mutation of G223S relative to CasX 515. In another embodiment, the disclosure provides CasX variant 668 (SEQ ID NO: 40344), which has an insertion of R at position 26 and a substitution of G223S relative to CasX 515. In another embodiment, the disclosure provides CasX 672 (SEQ ID NO: 40347), which has substitutions of L169K and G223S relative to CasX 515. In another embodiment, the disclosure provides CasX 676 (SEQ ID NO: 40351), which has substitutions of L169K and G223S and an insertion of R at position 26 relative to CasX 515. CasX variants with improved characteristics relative to CasX 515 include variants of Table 3.

Exemplary characteristics that can be improved in CasX variant proteins relative to the same characteristics in reference CasX proteins or relative to the CasX variant from which they were derived include, but are not limited to improved folding of the variant, increased binding affinity to the gRNA, increased binding affinity to the target nucleic acid, improved ability to utilize a greater spectrum of PAM sequences in the editing and/or binding of target nucleic acid, improved unwinding of the target DNA, increased editing activity, improved editing efficiency, improved editing specificity for the target nucleic acid, decreased off-target editing or cleavage, increased percentage of a eukaryotic genome that can be efficiently edited, increased activity of the nuclease, increased target strand loading for double strand cleavage, decreased target strand loading for single strand nicking, increased binding of the non-target strand of DNA, improved protein stability, improved protein:gRNA (RNP) complex stability, and improved fusion characteristics. In a particular embodiment, as described in the Examples, such improved characteristics can include, but are not limited to, improved cleavage activity in target nucleic acids having TTC, ATC, and CTC PAM sequences, increased specificity for cleavage of a target nucleic acid sequence, and decreased off-target cleavage of a target nucleic acid.

TABLE 6
CasX 515 domain sequences

| Domain | SEQ ID NO | Amino Acid Sequence |
|---|---|---|
| OBD-I | 40995 | QEIKRINKIRRRLVKDSNTKKAGKTGPMKTLLVRVMTPDLRERLE |
| | 48122 | NLRKKPENIPQ |
| Helical I-I | 40996 | PISNTSRANLNKLLTDYTEMKKAILHVYWEEFQKDPVGLMSRVA |
| | 41824 | |
| NTSB | 40988 | QPASKKIDQNKLKPEMDEKGNLTTAGFACSQCGQPLFVYKLEQV |
| | 41818 | SEKGKAYTNYFGRCNVAEHEKLILLAQLKPEKDSDEAVTYSLGKF |
| | | GQ |
| Helical I-II | 40989 | RALDFYSIHVTKESTHPVKPLAQIAGNRYASGPVGKALSDACMGT |
| | 40819 | IASFLSKYQDIIIEHQKVVKGNQKRLESLRELAGKENLEYPSVTLPP |
| | | QPHTKEGVDAYNEVIARVRMWVNLNLWQKLKLSRDDAKPLLRL |
| | | KGFPSF |
| Helical II | 41004 | PLVERQANEVDWWDMVCNVKKLINEKKEDGKVFWQNLAGYKR |
| | 41820 | QEALRPYLSSEEDRKKGKKFARYQLGDLLLHLEKKHGEDWGKV |
| | | YDEAWERIDKKVEGLSKHIKLEEERRSEDAQSKAALTDWLRAKA |
| | | SFVIEGLKEADKDEFCRCELKLQKWYGDLRGKPFAIEAE |
| OBD-II | 41000 | NSILDISGFSKQYNCAFIWQKDGVKKLNLYLIINYFKGGKLRFKKI |
| | 41823 | KPEAFEANRFYTVINKKSGEIVPMEVNFNFDDPNLIILPLAFGKRQ |
| | | GREFIWNDLLSLETGSLKLANGRVIEKTLYNRRTRQDEPALFVAL |
| | | TFERREVLD |
| RuvC-I | 41005 | SSNIKPMNLIGVDRGENIPAVIALTDPEGCPLSRFKDSLGNPTHILRI |
| | 41812 | GESYKEKQRTIQAKKEVEQRRAGGYSRKYASKAKNLADDMVRN |
| | | TARDLLYYAVTQDAMLIFENLSRGFGRQGKRTFMAERQYTRMED |
| | | WLTAKLAYEGLPSKTYLSKTLAQYTSKTC |
| TSL | 41002 | SNCGFTITSADYDRVLEKLKKTATGWMTTINGKELKVEGQITYYN |
| | 41825 | RYKRQNVVKDLSVELDRLSEESVNNDISSWTKGRSGEALSLLKKR |
| | | FSHRPVQEKFVCLNCGFETH |
| RuvC-II | 41003 | ADEQAALNIARSWLFLRSQEYKKYQTNKTTGNTDKRAFVETWQS |
| | 41826 | FYRKKLKEVWKPAV |

The CasX variants of the embodiments described herein have the ability to form an RNP complex with the gRNA disclosed herein. In some embodiments, an RNP comprising the CasX variant protein and a gRNA of the disclosure, at a concentration of 20 pM or less, is capable of cleaving a double stranded DNA target with an efficiency of at least 80%. In some embodiments, the RNP at a concentration of 20 pM or less is capable of cleaving a double stranded DNA target with an efficiency of at least 40%, at least 50%, at least 60%, at least 70%, at least 80%, at least 85%, at least 90% or at least 95%. In some embodiments, the RNP at a concentration of 50 pM or less, 40 pM or less, 30 pM or less, 20 pM or less, 10 pM or less, or 5 pM or less, is capable of cleaving a double stranded DNA target with an efficiency of at least 40%, at least 50%, at least 60%, at least 70%, at least 80%, at least 85%, at least 90% or at least 95%. These improved characteristics are described in more detail, below.

k. Catalytically-Dead CasX Variants

In some embodiments, for example those embodiments encompassing applications where cleavage of the target nucleic acid sequence is not a desired outcome, improving the catalytic activity of a CasX variant protein comprises altering, reducing, or abolishing the catalytic activity of the CasX variant protein. In some embodiments, the disclosure provides catalytically-dead CasX variant proteins that, while able to bind a target nucleic acid when complexed with a gRNA having a targeting sequence complementary to the target nucleic acid, are not able to cleave the target nucleic acid. Exemplary catalytically-dead CasX proteins comprise one or more mutations in the active site of the RuvC domain of the CasX protein. In some embodiments, a catalytically-dead CasX variant protein comprises substitutions at residues 672, 769 and/or 935 relative to SEQ ID NO: 1. In one embodiment, a catalytically-dead CasX variant protein comprises substitutions of D672A, E769A and/or D935A relative to a reference CasX protein of SEQ ID NO: 1. In other embodiments, a catalytically-dead CasX variant protein comprises substitutions at amino acids 659, 756 and/or 922 relative to a reference CasX protein of SEQ ID NO: 2. In some embodiments, a catalytically-dead CasX variant protein comprises D659A, E756A and/or D922A substitutions relative to a reference CasX protein of SEQ ID NO: 2. In some embodiments, a catalytically-dead CasX variant 527, 668 and 676 proteins comprise D660A, E757A, and D922A modifications to abolish the endonuclease activity. In further embodiments, a catalytically-dead CasX protein comprises deletions of all or part of the RuvC domain of the CasX protein. It will be understood that the same foregoing substitutions can similarly be introduced into the CasX variants of the disclosure, resulting in a catalytically-dead CasX (dCasX) variant. In one embodiment, all or a portion of the RuvC domain is deleted from the CasX variant, resulting in a dCasX variant. Catalytically inactive dCasX variant proteins can, in some embodiments, be used for base editing or epigenetic modifications. With a higher affinity for DNA, in some embodiments, catalytically inactive dCasX variant proteins can, relative to catalytically active CasX, find their target nucleic acid faster, remain bound to target nucleic acid for longer periods of time, bind target nucleic acid in a more stable fashion, or a combination thereof, thereby improving these functions of the catalytically-dead CasX variant protein compared to a CasX variant that retains its cleavage capability. Exemplary dCasX variant sequences are disclosed as SEQ ID NOS: 40808-40827 and 41006-41009 as set forth in Table 7. In some embodiments, a dCasX variant is at least 80% identical, at least 85% identical, at least 90% identical, at least 95% identical, at least 96% identical, at least 97% identical, at least 98% identical, or at least 99% identical to a sequence of SEQ ID NOS: 40808-40827, 41006-41009 and retains the functional properties of a dCasX variant protein. In some embodiments, a dCasX variant comprises a sequence of SEQ ID NOS: 40808-40827, 41006-41009.

TABLE 7
Catalytically-dead CasX Variant Proteins

| SEQ ID NO: | dCasX | Amino Acid Sequence |
|---|---|---|
| 40808 | CAS100 | — |
| 40809 | CAS099 | — |
| | [d491] | |
| 40810 | CAS098 | — |
| 40811 | CAS085 | — |
| 40812 | CAS087 | — |
| 40813 | CAS086 | — |
| 40814 | CAS083 | — |
| 40815 | CAS082 | — |
| 40816 | CAS069 | — |
| 40817 | CAS068 | — |
| 40818 | CAS070 | — |
| 40819 | CAS071 | — |
| 40820 | CAS072 | — |
| 40821 | CAS073 | — |
| 40822 | CAS074 | — |
| 40823 | CAS075 | — |
| 40824 | CAS076 | — |
| 40825 | CAS077 | — |
| 40826 | CAS078 | — |
| 40827 | CAS081 | — |
| 41006 | CAS096 | — |
| 41007 | CAS401 | — |
| 41008 | CAS142 | — |
| 41009 | CAS402 | — |

l. CasX Fusion Proteins

In some embodiments, the disclosure provides AAV encoding CasX proteins comprising a heterologous protein fused to the CasX. In some cases, the CasX is a CasX variant of any of the embodiments described herein. In some embodiments, a CasX variant comprises any one of the sequences as set forth in Table 3 fused to one or more proteins or domains thereof with an activity of interest.

In some embodiments, the CasX fusion protein comprises any one of the variants SEQ ID NOS: 49-160, 40208-40369, or 40828-40912 as set forth in Table 3, fused to one or more proteins or domains thereof that have a different activity of interest, resulting in a fusion protein. For example, in some embodiments, the CasX variant protein is fused to a protein (or domain thereof) that inhibits transcription, modifies a target nucleic acid, or modifies a polypeptide associated with a nucleic acid (e.g., histone modification).

In some embodiments, a heterologous polypeptide (or heterologous amino acid such as a cysteine residue or a non-natural amino acid) can be inserted at one or more positions within a CasX protein to generate a CasX fusion protein. In other embodiments, a cysteine residue can be inserted at one or more positions within a CasX protein followed by conjugation of a heterologous polypeptide described below. In some alternative embodiments, a heterologous polypeptide or heterologous amino acid can be added at the N- or C-terminus of the CasX variant protein. In other embodiments, a heterologous polypeptide or heterologous amino acid can be inserted internally within the sequence of the CasX protein.

In some embodiments, the CasX variant fusion protein retains RNA-guided sequence specific target nucleic acid binding and cleavage activity. In some cases, the CasX variant fusion protein has (retains) 50% or more of the activity (e.g., cleavage and/or binding activity) of the corresponding CasX variant protein that does not have the insertion of the heterologous protein. In some cases, the CasX variant fusion protein retains at least about 60%, or at least about 70% or more, at least about 80%, or at least about 90%, or at least about 92%, or at least about 95%, or at least about 98%, or at least about 100% of the activity (e.g., cleavage and/or binding activity) of the corresponding CasX protein that does not have the insertion of the heterologous protein.

In some cases, the CasX variant fusion protein retains (has) target nucleic acid binding activity relative to the activity of the CasX protein without the inserted heterologous amino acid or heterologous polypeptide. In some cases, the CasX variant fusion protein retains at least about 60%, or at least about 70% or more, at least about 80%, or at least about 90%, or at least about 92%, or at least about 95%, or at least about 98%, or at least about 100% of the binding activity of the corresponding CasX protein that does not have the insertion of the heterologous protein.

In some cases, the CasX variant fusion protein retains (has) target nucleic acid binding and/or cleavage activity relative to the activity of the parent CasX protein without the inserted heterologous amino acid or heterologous polypeptide. For example, in some cases, the CasX variant fusion protein has (retains) 50% or more of the binding and/or cleavage activity of the corresponding parent CasX protein (the CasX protein that does not have the insertion). For example, in some cases, the CasX variant fusion protein has (retains) 60% or more (70% or more, 80% or more, 90% or more, 92% or more, 95% or more, 98% or more, or 100%) of the binding and/or cleavage activity of the corresponding CasX parent protein (the CasX protein that does not have the insertion). Methods of measuring cleaving and/or binding activity of a CasX protein and/or a CasX fusion protein will be known to one of ordinary skill in the art and any convenient method can be used.

A variety of heterologous polypeptides are suitable for inclusion in a reference CasX or CasX variant fusion protein of the disclosure. In some cases, the fusion partner can modulate transcription (e.g., inhibit transcription, increase transcription) of a target DNA. For example, in some cases the fusion partner is a protein (or a domain from a protein) that inhibits transcription (e.g., a transcriptional repressor, a protein that functions via recruitment of transcription inhibitor proteins, modification of target DNA such as methylation, recruitment of a DNA modifier, modulation of histones associated with target DNA, recruitment of a histone modifier such as those that modify acetylation and/or methylation of histones, and the like). In some cases the fusion partner is a protein (or a domain from a protein) that increases transcription (e.g., a transcription activator, a protein that acts via recruitment of transcription activator proteins, modification of target DNA such as demethylation, recruitment of a DNA modifier, modulation of histones associated with target DNA, recruitment of a histone modifier such as those that modify acetylation and/or methylation of histones, and the like).

In some cases, a fusion partner has enzymatic activity that modifies a target nucleic acid sequence; e.g., nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity or glycosylase activity. In some embodiments, a CasX variant comprises any one of SEQ ID NOS: 49-160, 40208-40369, or 40828-40912 as set forth in Table 3 and a polypeptide with methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity or demyristoylation activity.

Examples of proteins (or fragments thereof) that can be used as a fusion partner to increase transcription include but are not limited to: transcriptional activators such as VP16, VP64, VP48, VP160, p65 subdomain (e.g., from NFkB), and activation domain of EDLL and/or TAL activation domain (e.g., for activity in plants); histone lysine methyltransferases such as SET1A, SET1B, MLL1 to 5, ASH1, SYMD2, NSD1, and the like; histone lysine demethylases such as JHDM2a/b, UTX, JMJD3, and the like; histone acetyltransferases such as GCN5, PCAF, CBP, p300, TAF1, TIP60/PLIP, MOZ/MYST3, MORF/MYST4, SRC1, ACTR, P160, CLOCK, and the like; and DNA demethylases such as Ten-Eleven Translocation (TET) dioxygenase 1 (TETICD), TET1, DME, DML1, DML2, ROS1, and the like.

Examples of proteins (or fragments thereof) that can be used as a fusion partner to decrease transcription include but are not limited to: transcriptional repressors such as the Kruppel associated box (KRAB or SKD); KOX1 repression domain; the Mad mSIN3 interaction domain (SID); the ERF repressor domain (ERD), the SRDX repression domain (e.g., for repression in plants), and the like; histone lysine methyltransferases such as Pr-SET7/8, SUV4-20H1, RIZ1, and the like; histone lysine demethylases such as JMJD2A/JHDM3A, JMJD2B, JMJD2C/GASC1, JMJD2D, JARID1A/RBP2, JARID1B/PLU-1, JARID 1C/SMCX, JARID1D/SMCY, and the like; histone lysine deacetylases such as HDAC1, HDAC2, HDAC3, HDAC8, HDAC4, HDAC5, HDAC7, HDAC9, SIRT1, SIRT2, HDAC11, and the like; DNA methylases such as Hhal DNA m5c-methyltransferase (M.Hhal), DNA methyltransferase 1 (DNMT1), DNA methyltransferase 3a (DNMT3a), DNA methyltransferase 3b (DNMT3b), METI, DRM3 (plants), ZMET2, CMT1, CMT2 (plants), and the like; and periphery recruitment elements such as Lamin A, Lamin B, and the like.

In some cases, the fusion partner has enzymatic activity that modifies the target nucleic acid sequence (e.g., ssRNA, dsRNA, ssDNA, dsDNA). Examples of enzymatic activity that can be provided by the fusion partner include but are not limited to: nuclease activity such as that provided by a restriction enzyme (e.g., FokI nuclease), methyltransferase activity such as that provided by a methyltransferase (e.g., Hhal DNA m5c-methyltransferase (M.Hhal), DNA methyltransferase 1 (DNMT1), DNA methyltransferase 3a (DNMT3a), DNA methyltransferase 3b (DNMT3b), METI, DRM3 (plants), ZMET2, CMT1, CMT2 (plants), and the like); demethylase activity such as that provided by a demethylase (e.g., Ten-Eleven Translocation (TET) dioxygenase 1 (TET 1 CD), TET1, DME, DML1, DML2, ROS1, and the like), DNA repair activity, DNA damage activity, deamination activity such as that provided by a deaminase (e.g., a cytosine deaminase enzyme, e.g., an APOBEC protein such as rat APOBECl), dismutase activity, alkylation activity, depurination activity, oxidation activity, pyrimidine dimer forming activity, integrase activity such as that provided by an integrase and/or resolvase (e.g., Gin invertase such as the hyperactive mutant of the Gin invertase, GinH106Y; human immunodeficiency virus type 1 integrase (IN); Tn3 resolvase; and the like), transposase activity, recombinase activity such as that provided by a recombinase (e.g., catalytic domain of Gin recombinase), polymerase activity, ligase activity, helicase activity, photolyase activity, and glycosylase activity).

In some cases, a CasX variant protein for use in the AAV systems of the present disclosure is fused to a polypeptide selected from a domain for increasing transcription (e.g., a VP16 domain, a VP64 domain), a domain for decreasing transcription (e.g., a KRAB domain, e.g., from the Kox1 protein), a core catalytic domain of a histone acetyltransferase (e.g., histone acetyltransferase p300), a protein/domain that provides a detectable signal (e.g., a fluorescent protein such as GFP), a nuclease domain (e.g., a Fokl nuclease), or a base editor (e.g., cytidine deaminase such as APOBEC1).

In some cases, the fusion partner has enzymatic activity that modifies a protein associated with the target nucleic acid (e.g., ssRNA, dsRNA, ssDNA, dsDNA) (e.g., a histone, an RNA binding protein, a DNA binding protein, and the like). Examples of enzymatic activity (that modifies a protein associated with a target nucleic acid) that can be provided by the fusion partner include but are not limited to: methyltransferase activity such as that provided by a histone methyltransferase (HMT) (e.g., suppressor of variegation 3-9 homolog 1 (SUV39H1, also known as KMT1A), euchromatic histone lysine methyltransferase 2 (G9A, also known as KMT1C and EHMT2), SUV39H2, ESET/SETDB 1, and the like, SET1A, SET1B, MLL1 to 5, ASH1, SYMD2, NSD1, DOT1L, Pr-SET7/8, SUV4-20H1, EZH2, RIZ1), demethylase activity such as that provided by a histone demethylase (e.g., Lysine Demethylase 1A (KDM1A also known as LSD1), JHDM2a/b, JMJD2A/JHDM3A, JMJD2B, JMJD2C/GASC1, JMJD2D, JARID1A/RBP2, JARID1B/PLU-1, JARID1C/SMCX, JARID1D/SMCY, UTX, JMJD3, and the like), acetyltransferase activity such as that provided by a histone acetylase transferase (e.g., catalytic core/fragment of the human acetyltransferase p300, GCN5, PCAF, CBP, TAF1, TIP60/PLIP, MOZ/MYST3, MORF/MYST4, HBO1/MYST2, HMOF/MYST1, SRC1, ACTR, P160, CLOCK, and the like), deacetylase activity such as that provided by a histone deacetylase (e.g., HDAC1, HDAC2, HDAC3, HDAC8, HDAC4, HDAC5, HDAC7, HDAC9, SIRT1, SIRT2, HDAC11, and the like), kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, and demyristoylation activity.

Additional examples of suitable fusion partners of the CasX variants are (i) a dihydrofolate reductase (DHFR) destabilization domain (e.g., to generate a chemically controllable subject RNA-guided polypeptide or a conditionally active RNA-guided polypeptide), and (ii) a chloroplast transit peptide.

In some embodiments, a CasX variant comprises any one of SEQ ID NOS: 49-160, 40208-40369, or 40828-40912 as set forth in Table 3, and a chloroplast transit peptide including, but are not limited to:

(SEQ ID NO: 40790)
MASMISSSAVTTVSRASRGQSAAMAPFGGLKSMTGFPVRKVNTDITSIT
SNGGRVKCMQVWPPIGKKKFETLSYLPPLTRDSRA;
(SEQ ID NO: 39980)
MASMISSSAVTTVSRASRGQSAAMAPFGGLKSMTGFPVRKVNTDITSIT
SNGGRVKS;
(SEQ ID NO: 39968)
MASSMLSSATMVASPAQATMVAPFNGLKSSAAFPATRKANNDITSITSN
GGRVNCMQVWPPIEKKKFETLSYLPDLTDSGGRVNC;
(SEQ ID NO: 39969)
MAQVSRICNGVQNPSLISNLSKSSQRKSPLSVSLKTQQHPRAYPISSSW
GLKKSGMTLIGSELRPLKVMSSVSTAC;
(SEQ ID NO: 39970)
MAQVSRICNGVWNPSLISNLSKSSQRKSPLSVSLKTQQHPRAYPISSSW
GLKKSGMTLIGSELRPLKVMSSVSTAC;
(SEQ ID NO: 39971)
MAQINNMAQGIQTLNPNSNFHKPQVPKSSSFLVFGSKKLKNSANSMLVL
KKDSIFMQLFCSFRISASVATAC;
(SEQ ID NO: 39972)
MAALVTSQLATSGTVLSVTDRFRRPGFQGLRPRNPADAALGMRTVGASA
APKQSRKPHRFDRRCLSMVV;
(SEQ ID NO: 39973)
MAALTTSQLATSATGFGIADRSAPSSLLRHGFQGLKPRSPAGGDATSLS
VTTSARATPKQQRSVQRGSRRFPSVVVC;
(SEQ ID NO: 39974)
MASSVLSSAAVATRSNVAQANMVAPFTGLKSAASFPVSRKQNLDITSIA
SNGGRVQC;
(SEQ ID NO: 39975)
MESLAATSVFAPSRVAVPAARALVRAGTVVPTRRTSSTSGTSGVKCSAA
VTPQASPVISRSAAAA;
and
(SEQ ID NO: 39976)
MGAAATSMQSLKFSNRLVPPSRRLSPVPNNVTCNNLPKSAAPVRTVKCC
ASSWNSTINGAAATTNGASAASS.

In some cases, a CasX variant protein of the present disclosure for use in the AAV systems can include an endosomal escape peptide. In some cases, an endosomal escape polypeptide comprises the amino acid sequence GLFXALLXLLXSLWXLLLXA (SEQ ID NO: 39977), wherein each X is independently selected from lysine, histidine, and arginine. In some cases, an endosomal escape polypeptide comprises the amino acid sequence GLFHALLHLLHSLWHLLLHA (SEQ ID NO: 39978), or HHHHHHHHH (SEQ ID NO: 39979).

Non-limiting examples of fusion partners for use with a CasX variant when targeting ssRNA target nucleic acid sequences include (but are not limited to): splicing factors (e.g., RS domains); protein translation components (e.g., translation initiation, elongation, and/or release factors; e.g., eIF4G); RNA methylases; RNA editing enzymes (e.g., RNA deaminases, e.g., adenosine deaminase acting on RNA (ADAR), including A to I and/or C to U editing enzymes); helicases; RNA-binding proteins; and the like. It is understood that a heterologous polypeptide can include the entire protein or in some cases can include a fragment of the protein (e.g., a functional domain).

In some embodiments, a CasX variant of any one of SEQ ID NOS: 49-160, 40208-40369, or 40828-40912 as set forth in Table 3, comprises a fusion partner of any domain capable of interacting with ssRNA (which, for the purposes of this disclosure, includes intramolecular and/or intermolecular secondary structures, e.g., double-stranded RNA duplexes such as hairpins, stem-loops, etc.), whether transiently or irreversibly, directly or indirectly, including but not limited to an effector domain selected from the group comprising; endonucleases (for example RNase III, the CRR22 DYW domain, Dicer, and PIN (PilT N-terminus) domains from proteins such as SMG5 and SMG6); proteins and protein domains responsible for stimulating RNA cleavage (for example CPSF, CstF, CFIm and CFIIm); exonucleases (for example XRN-1 or Exonuclease T); deadenylases (for example HNT3); proteins and protein domains responsible for nonsense mediated RNA decay (for example UPF1, UPF2, UPF3, UPF3b, RNP SI, Y14, DEK, REF2, and SRm160); proteins and protein domains responsible for stabilizing RNA (for example PABP); proteins and protein domains responsible for repressing translation (for example Ago2 and Ago4); proteins and protein domains responsible for stimulating translation (for example Staufen); proteins and protein domains responsible for (e.g., capable of) modulating translation (e.g., translation factors such as initiation factors, elongation factors, release factors, etc., e.g., eIF4G); proteins and protein domains responsible for polyadenylation of RNA (for example PAP1, GLD-2, and Star-PAP); proteins and protein domains responsible for polyuridinylation of RNA (for example CI Dl and terminal uridylate transferase); proteins and protein domains responsible for RNA localization (for example from IMPI, ZBP1, She2p, She3p, and Bicaudal-D); proteins and protein domains responsible for nuclear retention of RNA (for example Rrp6); proteins and protein domains responsible for nuclear export of RNA (for example TAP, NXF1, THO, TREX, REF, and Aly); proteins and protein domains responsible for repression of RNA splicing (for example PTB, Sam68, and hnRNP A1); proteins and protein domains responsible for stimulation of RNA splicing (for example serine/arginine-rich (SR) domains); proteins and protein domains responsible for reducing the efficiency of transcription (for example FUS (TLS)); and proteins and protein domains responsible for stimulating transcription (for example CDK7 and HIV Tat). Alternatively, the effector domain may be selected from the group comprising endonucleases; proteins and protein domains capable of stimulating RNA cleavage; exonucleases; deadenylases; proteins and protein domains having nonsense mediated RNA decay activity; proteins and protein domains capable of stabilizing RNA; proteins and protein domains capable of repressing translation; proteins and protein domains capable of stimulating translation; proteins and protein domains capable of modulating translation (e.g., translation factors such as initiation factors, elongation factors, release factors, etc., e.g., eIF4G); proteins and protein domains capable of polyadenylation of RNA; proteins and protein domains capable of polyuridinylation of RNA; proteins and protein domains having RNA localization activity; proteins and protein domains capable of nuclear retention of RNA; proteins and protein domains having RNA nuclear export activity; proteins and protein domains capable of repression of RNA splicing; proteins and protein domains capable of stimulation of RNA splicing; proteins and protein domains capable of reducing the efficiency of transcription; and proteins and protein domains capable of stimulating transcription. Another suitable heterologous polypeptide is a PUF RNA-binding domain, which is described in more detail in WO2012068627, which is hereby incorporated by reference in its entirety.

Some RNA splicing factors that can be used (in whole or as fragments thereof) as a fusion partner for a CasX variant have modular organization, with separate sequence-specific RNA binding modules and splicing effector domains. For example, members of the serine/arginine-rich (SR) protein family contain N-terminal RNA recognition motifs (RRMs) that bind to exonic splicing enhancers (ESEs) in pre-mRNAs and C-terminal RS domains that promote exon inclusion. As another example, the hnRNP protein hnRNP A1 binds to exonic splicing silencers (ESSs) through its RRM domains and inhibits exon inclusion through a C-terminal glycine-rich domain. Some splicing factors can regulate alternative use of splice site (ss) by binding to regulatory sequences between the two alternative sites. For example, ASF/SF2 can recognize ESEs and promote the use of intron proximal sites, whereas hnRNP A1 can bind to ESSs and shift splicing towards the use of intron distal sites. One application for such factors is to generate ESFs that modulate alternative splicing of endogenous genes, particularly disease associated genes. For example, Bcl-x pre-mRNA produces two splicing isoforms with two alternative 5′ splice sites to encode proteins of opposite functions. The long splicing isoform Bcl-xL is a potent apoptosis inhibitor expressed in long-lived post mitotic cells and is up-regulated in many cancer cells, protecting cells against apoptotic signals. The short isoform Bcl-xS is a pro-apoptotic isoform and expressed at high levels in cells with a high turnover rate (e.g., developing lymphocytes). The ratio of the two Bcl-x splicing isoforms is regulated by multiple cis-elements that are located in either the core exon region or the exon extension region (i.e., between the two alternative 5′ splice sites). For more examples, see WO2010075303, which is hereby incorporated by reference in its entirety.

Further suitable fusion partners for use with a CasX variant include, but are not limited to, proteins (or fragments thereof) that are boundary elements (e.g., CTCF), proteins and fragments thereof that provide periphery recruitment (e.g., Lamin A, Lamin B, etc.), and protein docking elements (e.g., FKBP/FRB, Pill/Abyl, etc.).

In some cases, a heterologous polypeptide (a fusion partner) for use with a CasX variant provides for subcellular localization, i.e., the heterologous polypeptide contains a subcellular localization sequence (e.g., a nuclear localization signal (NLS) for targeting to the nucleus, a sequence to keep the fusion protein out of the nucleus, e.g., a nuclear export sequence (NES), a sequence to keep the fusion protein retained in the cytoplasm, a mitochondrial localization signal for targeting to the mitochondria, a chloroplast localization signal for targeting to a chloroplast, an ER retention signal, and the like). In some embodiments, a subject RNA-guided polypeptide or a conditionally active RNA-guided polypeptide and/or subject CasX fusion protein does not include a NLS so that the protein is not targeted to the nucleus (which can be advantageous, e.g., when the target nucleic acid sequence is an RNA that is present in the cytosol). In some embodiments, a fusion partner can provide a tag (i.e., the heterologous polypeptide is a detectable label) for ease of tracking and/or purification (e.g., a fluorescent protein, e.g., green fluorescent protein (GFP), yellow fluorescent protein (YFP), red fluorescent protein (RFP), cyan fluorescent protein (CFP), mCherry, tdTomato, and the like; a histidine tag, e.g., a 6×His tag; a hemagglutinin (HA) tag; a FLAG tag; a Myc tag; and the like).

In some cases, a CasX variant protein for use in the AAV systems includes (is fused to) a nuclear localization signal (NLS) for targeting the CasX/gRNA to the nucleus of the cell. In some cases, a CasX variant protein is fused to 2 or more, 3 or more, 4 or more, or 5 or more 6 or more, 7 or more, 8 or more NLSs. In some cases, one or more NLSs (2 or more, 3 or more, 4 or more, or 5 or more NLSs) are positioned at or near (e.g., within 50 amino acids of) the N-terminus and/or the C-terminus. In some cases, one or more NLSs (2 or more, 3 or more, 4 or more, or 5 or more NLSs) are positioned at or near (e.g., within 50 amino acids of) the N-terminus. In some cases, one or more NLSs (2 or more, 3 or more, 4 or more, or 5 or more NLSs) are positioned at or near (e.g., within 50 amino acids of) the C-terminus. In some cases, an NLS is positioned at the N-terminus and an NLS is positioned at the C-terminus. In some cases, one or more NLSs (2 or more, 3 or more, 4 or more, or 5 or more NLSs) are positioned at or near (e.g., within 50 amino acids of) both the N-terminus and the C-terminus. In some cases, a CasX variant protein includes (is fused to) between 1 and 10 NLSs (e.g., 1-9, 1-8, 1-7, 1-6, 1-5, 2-10, 2-9, 2-8, 2-7, 2-6, or 2-5 NLSs). In some cases, a CasX variant protein includes (is fused to) between 2 and 5 NLSs (e.g., 2-4, or 2-3 NLSs). Non-limiting examples of NLSs suitable for use with a CasX variant include sequences having at least about 80%, at least about 90%, or at least about 95% identity or are identical to sequences derived from: the NLS of the SV40 virus large T-antigen, having the amino acid sequence PKKKRKV (SEQ ID NO: 196); the NLS from nucleoplasmin (e.g., the nucleoplasmin bipartite NLS with the sequence KRPAATKKAGQAKKKK (SEQ ID NO: 197); the c-myc NLS having the amino acid sequence PAAKRVKLD (SEQ ID NO: 248) or RQRRNELKRSP (SEQ ID NO: 161); the hRNPA1 M9 NLS having the sequence NQSSNFGPMKGGNFGGRSSGPYGGGGQYFAKPRNQGGY (SEQ ID NO: 162); the sequence RMRIZFKNKGKDTAELRRRRVEVSVELRKAKKDEQILKRRNV (SEQ ID NO: 163) of the IBB domain from importin-alpha; the sequences VSRKRPRP (SEQ ID NO: 164) and PPKKARED (SEQ ID NO: 165) of the myoma T protein; the sequence PQPKKKPL (SEQ ID NO: 166) of human p53; the sequence SALIKKKKKMAP (SEQ ID NO: 167) of mouse c-abl IV; the sequences DRLRR (SEQ ID NO: 168) and PKQKKRK (SEQ ID NO: 169) of the influenza virus NS1; the sequence RKLKKKIKKL (SEQ ID NO: 170) of the Hepatitis virus delta antigen; the sequence REKKKFLKRR (SEQ ID NO: 171) of the mouse Mxl protein; the sequence KRKGDEVDGVDEVAKKKSKK (SEQ ID NO: 172) of the human poly(ADP-ribose) polymerase; the sequence RKCLQAGMNLEARKTKK (SEQ ID NO: 173) of the steroid hormone receptors (human) glucocorticoid; the sequence PRPRKIPR (SEQ ID NO: 174) of Borna disease virus P protein (BDV-P1); the sequence PPRKKRTVV (SEQ ID NO: 175) of hepatitis C virus nonstructural protein (HCV-NS5A); the sequence NLSKKKKRKREK (SEQ ID NO: 176) of LEF1; the sequence RRPSRPFRKP (SEQ ID NO: 177) of ORF57 simirae; the sequence KRPRSPSS (SEQ ID NO: 178) of EBV LANA; the sequence KRGINDRNFWRGENERKTR (SEQ ID NO: 179) of Influenza A protein; the sequence PRPPKMARYDN (SEQ ID NO: 180) of human RNA helicase A (RHA); the sequence KRSFSKAF (SEQ ID NO: 181) of nucleolar RNA helicase II; the sequence KLKIKRPVK (SEQ ID NO: 182) of TUS-protein; the sequence PKKKRKVPPPPAAKRVKLD (SEQ ID NO: 183) associated with importin-alpha; the sequence PKTRRRPRRSQRKRPPT (SEQ ID NO: 184) from the Rex protein in HTLV-1; the sequence MSRRRKANPTKLSENAKKLAKEVEN (SEQ ID NO: 185) from the EGL-13 protein of Caenorhabditis elegans; and the sequences KTRRRPRRSQRKRPPT (SEQ ID NO: 186), RRKKRRPRRKKRR (SEQ ID NO: 187), PKKKSRKPKKKSRK (SEQ ID NO: 188), HKKKHPDASVNFSEFSK (SEQ ID NO: 189), QRPGPYDRPQRPGPYDRP (SEQ ID NO: 190), LSPSLSPLLSPSLSPL (SEQ ID NO: 191), RGKGGKGLGKGGAKRHRK (SEQ ID NO: 192), PKRGRGRPKRGRGR (SEQ ID NO: 193), PKKKRKVPPPPAAKRVKLD (SEQ ID NO: 194) and PKKKRKVPPPPKKKRKV (SEQ ID NO: 195), PAKRARRGYKC (SEQ ID NO: 40188), KLGPRKATGRW (SEQ ID NO: 40189), PRRKREE (SEQ ID NO: 40190), PYRGRKE (SEQ ID NO: 40191), PLRKRPRR (SEQ ID NO: 40192), PLRKRPRRGSPLRKRPRR (SEQ ID NO: 40193), PAAKRVKLDGGKRTADGSEFESPKKKRKV (SEQ ID NO: 40194), PAAKRVKLDGGKRTADGSEFESPKKKRKVGIHGVPAA (SEQ ID NO: 40195), PAAKRVKLDGGKRTADGSEFESPKKKRKVAEAAAKEAAAKEAAAKA (SEQ ID NO: 40196), PAAKRVKLDGGKRTADGSEFESPKKKRKVPG (SEQ ID NO: 40197), KRKGSPERGERKRHW (SEQ ID NO: 40198), KRTADSQHSTPPKTKRKVEFEPKKKRKV (SEQ ID NO: 40199), and PKKKRKVGGSKRTADSQHSTPPKTKRKVEFEPKKKRKV (SEQ ID NO: 40200). Additional NLS for incorporation in the AAV systems of the disclosure are provided in Tables 15 and 16, indicating NLS for linking to the N- or C-terminus of the CasX. In some embodiments, the one or more NLS are linked to the CasX or to an adjacent NLS by a linker peptide wherein the linker peptide is selected from the group consisting of RS, (G)n (SEQ ID NO: 40201), (GS)n (SEQ ID NO: 40202), (GSGGS)n (SEQ ID NO: 208), (GGSGGS)n (SEQ ID NO: 209), (GGGS)n (SEQ ID NO: 210), GGSG (SEQ ID NO: 211), GGSGG (SEQ ID NO: 212), GSGSG (SEQ ID NO: 213), GSGGG (SEQ ID NO: 214), GGGSG (SEQ ID NO: 215), GSSSG (SEQ ID NO: 216), GPGP (SEQ ID NO: 217), GGP, PPP, PPAPPA (SEQ ID NO: 218), PPPG (SEQ ID NO: 40207), PPPGPPP (SEQ ID NO: 219), PPP(GGGS)n (SEQ ID NO: 40203), (GGGS)nPPP (SEQ ID NO: 40204), AEAAAKEAAAKEAAAKA (SEQ ID NO: 40205), and TPPKTKRKVEFE (SEQ ID NO: 40206), wherein n is 1 to 5. In some embodiments, the AAV constructs of the disclosure comprise polynucleic acids encoding the NLS and linker peptides of any of the foregoing embodiments of the paragraph, as well as the NLS of Tables 15 and 16, and can be, in some cases, configured in relation to the other components of the constructs as depicted in any one of FIG. 24, 33-35 or 42.

In general, NLS (or multiple NLSs) are of sufficient strength to drive accumulation of a CasX variant fusion protein in the nucleus of a eukaryotic cell. Detection of accumulation in the nucleus may be performed by any suitable technique. For example, a detectable marker may be fused to a CasX variant fusion protein such that location within a cell may be visualized. Cell nuclei may also be isolated from cells, the contents of which may then be analyzed by any suitable process for detecting protein, such as immunohistochemistry, Western blot, or enzyme activity assay. Accumulation in the nucleus may also be determined indirectly.

In some cases, a CasX variant fusion protein for use in the AAV systems includes a “protein transduction domain” or PTD (also known as a CPP—cell penetrating peptide), which refers to a protein, polynucleotide, carbohydrate, or organic or inorganic compound that facilitates traversing a lipid bilayer, micelle, cell membrane, organelle membrane, or vesicle membrane. A PTD attached to another molecule, which can range from a small polar molecule to a large macromolecule and/or a nanoparticle, facilitates the molecule traversing a membrane, for example going from an extracellular space to an intracellular space, or from the cytosol to within an organelle. In some embodiments, a PTD is covalently linked to the amino terminus of a CasX variant fusion protein. In some embodiments, a PTD is covalently linked to the carboxyl terminus of a CasX variant fusion protein. In some cases, the PTD is inserted internally in the sequence of a CasX variant fusion protein at a suitable insertion site. In some cases, a CasX variant fusion protein includes (is conjugated to, is fused to) one or more PTDs (e.g., two or more, three or more, four or more PTDs). In some cases, a PTD includes one or more nuclear localization signals (NLS). Examples of PTDs include, but are not limited to, peptide transduction domain of HIV TAT comprising YGRKKRRQRRR (SEQ ID NO: 198), RKKRRQRR (SEQ ID NO: 199); YARAAARQARA (SEQ ID NO: 200); THRLPRRRRRR (SEQ ID NO: 201); and GGRRARRRRRR (SEQ ID NO: 202); a polyarginine sequence comprising a number of arginines sufficient to direct entry into a cell (e.g., 3, 4, 5, 6, 7, 8, 9, 10, or 10-50 arginines) (SEQ ID NO: 203); a VP22 domain (Zender et al. (2002) Cancer Gene Ther. 9(6):489-96); an Drosophila Antennapedia protein transduction domain (Noguchi et al. (2003) Diabetes 52(7): 1732-1737); a truncated human calcitonin peptide (Trehin et al. (2004) Pharm. Research 21: 1248-1256); polylysine (Wender et al. (2000) Proc. Natl. Acad. Sci. USA 97: 13003-13008); RRQRRTSKLMKR (SEQ ID NO: 204); Transportan GWTLNSAGYLLGKINLKALAALAKKIL (SEQ ID NO: 205); KALAWEAKLAKALAKALAKHLAKALAKALKCEA (SEQ ID NO: 206); and RQIKIWFQNRRMKWKK (SEQ ID NO: 207). In some embodiments, the PTD is an activatable CPP (ACPP) (Aguilera et al. (2009) IntegrBiol (Camb) June; 1(5-6): 371-381). ACPPs comprise a polycationic CPP (e.g., Arg9 or “R9”) connected via a cleavable linker to a matching polyanion (e.g., Glu9 or “E9”), which reduces the net charge to nearly zero and thereby inhibits adhesion and uptake into cells. Upon cleavage of the linker, the polyanion is released, locally unmasking the polyarginine and its inherent adhesiveness, thus “activating” the ACPP to traverse the membrane.

In some embodiments, a CasX variant fusion protein can include a CasX protein that is linked to an internally inserted heterologous amino acid or heterologous polypeptide (a heterologous amino acid sequence) via a linker polypeptide (e.g., one or more linker polypeptides). In some embodiments, a CasX variant fusion protein can be linked at the C-terminal and/or N-terminal end to a heterologous polypeptide (fusion partner) via a linker polypeptide (e.g., one or more linker polypeptides). The linker polypeptide may have any of a variety of amino acid sequences. Proteins can be joined by a spacer peptide, generally of a flexible nature, although other chemical linkages are not excluded. Suitable linkers include polypeptides of between 4 amino acids and 40 amino acids in length, or between 4 amino acids and 25 amino acids in length. These linkers are generally produced by using synthetic, linker-encoding oligonucleotides to couple the proteins. Peptide linkers with a degree of flexibility can be used. The linking peptides may have virtually any amino acid sequence, bearing in mind that the preferred linkers will have a sequence that results in a generally flexible peptide. The use of small amino acids, such as glycine and alanine, are of use in creating a flexible peptide. The creation of such sequences is routine to those of skill in the art. A variety of different linkers are commercially available and are considered suitable for use. Example linker polypeptides include glycine polymers (G)n, glycine-serine polymers, glycine-alanine polymers, alanine-serine polymers, glycine-proline polymers, proline polymers and proline-alanine polymers. Example linkers can comprise amino acid sequences including, but not limited to (G)n (SEQ ID NO: 40201), (GS)n (SEQ ID NO: 40202), (GSGGS)n (SEQ ID NO: 208), (GGSGGS)n (SEQ ID NO: 209), (GGGS)n (SEQ ID NO: 210), GGSG (SEQ ID NO: 211), GGSGG (SEQ ID NO: 212), GSGSG (SEQ ID NO: 213), GSGGG (SEQ ID NO: 214), GGGSG (SEQ ID NO: 215), GSSSG (SEQ ID NO: 216), GPGP (SEQ ID NO: 217), GGP, PPP, PPAPPA (SEQ ID NO: 218), PPPG (SEQ ID NO: 40207), PPPGPPP (SEQ ID NO: 219), PPP(GGGS)n (SEQ ID NO: 40203), (GGGS)nPPP (SEQ ID NO: 40204), AEAAAKEAAAKEAAAKA (SEQ ID NO: 40205), and TPPKTKRKVEFE (SEQ ID NO: 40206), where n is 1 to 5, where n is 1 to 5. The ordinarily skilled artisan will recognize that design of a peptide conjugated to any elements described above can include linkers that are all or partially flexible, such that the linker can include a flexible linker as well as one or more portions that confer less flexible structure.

### V. AAV Systems and Methods for Modification of Target Nucleic Acids

The AAV provided herein are useful for various applications, including as therapeutics, diagnostics, and for research. To effect the methods of the disclosure for gene editing, provided herein are programmable AAV systems. The programmable nature of the CasX and gRNA components of the AAV systems provided herein allows for the precise targeting to achieve the desired effect (nicking, cleaving, etc.) at one or more regions of predetermined interest in the target nucleic acid sequence. In some embodiments, the AAV systems provided herein comprise sequences encoding a CasX protein and a gRNA wherein the targeting sequence of the gRNA is complementary to, and therefore is capable of hybridizing with, a target nucleic acid sequence. In some cases, the AAV system further comprises a donor template nucleic acid.

In some embodiments of the disclosure, provided herein are methods of modifying a target nucleic acid sequence. In some embodiments, the methods comprise contacting a cell comprising the target nucleic acid sequence with an AAV encoding a CasX protein of the disclosure and a gRNA of the disclosure comprising a targeting sequence, wherein the targeting sequence of the gRNA has a sequence complementary to and that can hybridize with the sequence of the target nucleic acid. Upon hybridization with the target nucleic acid by the CasX and the gRNA, the CasX introduces one or more single-strand breaks or double-strand breaks within or near the target nucleic acid, which may include sequences that contain regulatory elements or non-coding regions of the gene, that results in a permanent indel (deletion or insertion) or mutation in the target nucleic acid, as described herein, with a corresponding modulation of expression or alteration in the function of the gene product, thereby creating an edited cell. In other embodiments, the method comprises contacting a cell comprising the target nucleic acid sequence with an AAV encoding a plurality of gRNAs targeted to different or overlapping portions of the target nucleic acid wherein the CasX protein introduces multiple breaks in the target nucleic acid that result in a permanent indel or mutation in the target nucleic acid, as described herein, with a corresponding modulation of expression or alteration in the function of the gene product, thereby creating an edited cell.

In some embodiments, the modification of the target nucleic acid results in reduced expression of a gene product of a gene comprising the target nucleic acid, wherein expression is reduced by at least about 10%, at least about 20%, at least about 30%, at least about 40%, at least about 50%, at least about 60%, at least about 70%, at least about 80%, or at least about 90% in comparison to a cell that has not been modified.

In some embodiments of the method of modifying a target nucleic acid sequence, the gRNA of the AAV vector is a guide DNA (gDNA). In other embodiments, the gRNA is a guide RNA (gRNA). In some embodiments, the gRNA is a single-molecule gRNA (sgRNA). In other embodiments of the method, the gRNA is a dual-molecule gRNA (dgRNA) wherein the activator and the targeter components are linked together by intervening nucleotides. In some embodiments, the gRNA is a chimeric gRNA-gDNA. In some embodiments, the method comprises contacting the target nucleic acid sequence with and AAV encoding a plurality of gRNAs targeted to different or overlapping regions of the target nucleic acid. In some embodiments, the gRNA scaffold comprises any one of the sequences of SEQ ID NOS: 2101-2285, 39981-40026, 40913-40958, and 41817 as set forth in Table 2.

In some embodiments of the method of modifying a target nucleic acid sequence, the CasX protein incorporated into the AAV vector is a reference CasX selected from SEQ ID NOS: 1-3, or a CasX variant having at least 50%, at least 60%, at least 70%, at least 80%, or at least 90%, or at least 95%, or at least 99% sequence identity to the reference CasX proteins of SEQ ID NOS:1-3. In some embodiments, the CasX variant protein comprises at least one modification relative to a reference CasX protein having a sequence selected from SEQ ID NOS: 1-3. In some embodiments, the at least one modification comprises at least one amino acid substitution, deletion, or insertion in a domain relative to the reference CasX protein. In some embodiments, the at least one modification comprises at least one amino acid deletion in a domain relative to the reference CasX protein. In other embodiments, the at least one modification comprises at least one amino acid insertion in a domain relative to the reference CasX protein. In some embodiments, the at least one modification comprises at least one amino acid substitution in a domain relative to the reference CasX protein. In some embodiments of the methods, the AAV encodes a CasX variant having a sequence of SEQ ID NOS: 49-160, 40208-40369 and 40828-40912 as set forth in Table 3, or a sequence having at least about 50%, at least about 60%, at least about 70%, at least about 80%, at least about 90%, or at least about 95%, or at least about 96%, or at least about 97%, or at least about 98%, or at least about 99% sequence identity thereto. In the embodiments, the CasX variant protein exhibits at least one or more improved characteristics as compared to a reference CasX protein. In some embodiments of the method, the one or more improved characteristics of the CasX variant protein are selected from the group consisting of improved folding of the CasX protein, improved binding affinity to the guide RNA, improved binding affinity to the target nucleic acid sequence, altered binding affinity to one or more PAM sequences, ability to effectively bind a greater spectrum of canonical PAM sequences compared to reference CasX proteins, including TTC, ATC, GTC, and CTC, improved unwinding of the target nucleic acid sequence, increased activity, improved editing efficiency, improved editing specificity, increased activity of the nuclease, increased target strand loading for double strand cleavage, decreased target strand loading for single strand nicking, decreased off-target cleavage, improved binding of the non-target strand of DNA, improved protein stability, improved protein:guide RNA complex stability, improved protein solubility, improved protein:guide RNA complex solubility, improved protein yield, improved protein expression, and improved fusion characteristics. In some embodiments of the methods, the improved characteristic of the CasX variant protein is at least about 1.1 to about 100,000-fold improved relative to the reference protein of SEQ ID NO: 1, SEQ ID NO: 2, or SEQ ID NO: 3. In some embodiments, the improved characteristic of the CasX variant protein is at least about 10 to about 10,000-fold improved relative to the reference protein of SEQ ID NO: 1, SEQ ID NO: 2, or SEQ ID NO:3. In some embodiments, the improved characteristic of the CasX variant protein is at least about 1.1 to about 1000-fold increased binding affinity of the CasX protein to the gRNA compared to the protein of SEQ ID NO: 1, SEQ ID NO: 2, or SEQ ID NO: 3. In some embodiments, the improved characteristic of the CasX variant protein is at least about 1.1, at least 1.5, at least 10, at least 50, at least 100, at least 500, at least 1,000, at least 5,000, or at least a 10,000-fold improved, as compared to a reference CasX protein of SEQ ID NO: 1, SEQ ID NO: 2, or SEQ ID NO: 3. In some embodiments, the CasX variant protein has at least about 1.1 to about 10-fold increased binding affinity to the target nucleic acid sequence compared to the protein of SEQ ID NO: 1, SEQ ID NO: 2, or SEQ ID NO: 3. In some embodiments, the increased binding affinity to the target nucleic acid sequence by the CasX variant protein is to one or more PAM sequences, including TTC, ATC, GTC, and CTC,

In some embodiments, the modifying of the target nucleic acid sequence is carried out ex vivo. In some embodiments, the modifying of the target nucleic acid sequence is carried out in vitro inside a cell. In some embodiments of the modification of the target nucleic acid sequence in a cell, the cell is a eukaryotic cell selected from the group consisting of a rodent cell, a mouse cell, a rat cell, a primate cell, a non-human primate cell, and a human cell. In particular embodiments, the eukaryotic cell is a human cell. In some embodiments, the modifying of the target nucleic acid sequence is carried out in vivo in a subject. In some embodiments, the subject is selected from the group consisting of mouse, rat, pig, non-human primate, and human.

In some embodiments, the method of modifying a target nucleic acid sequence comprises contacting a target nucleic acid with an AAV vector encoding a CasX protein and gRNA pair and further comprising a donor template. The donor template may be inserted into the target nucleic acid such that all, some or none of the gene product is expressed. Depending on whether the system is used to knock-down/knock-out or to knock-in a protein-coding sequence, the donor template can be a short single-stranded or double-stranded oligonucleotide, or can be a long single-stranded or double-stranded oligonucleotide. For knock-down/knock-outs, the donor template sequence need not be identical to the genomic sequence that it replaces and may contain one or more single base changes, insertions, deletions, inversions or rearrangements with respect to the genomic sequence. Provided that there are arms with sufficient numbers of nucleotides having sufficient homology flanking the cleavage site(s) of the target nucleic acid sequence targeted by the CasX:gRNA (i.e., 5′ and 3′ to the cleavage site) to support homology-directed repair (“homologous arms”), use of such donor templates can result in a frame-shift or other mutation such that the gene product is not expressed or is expressed at a lower level. In some embodiments, the homologous arms comprise between 10 and 100 nucleotides. The upstream and downstream homology arm sequences share at least about 80%, 85%, 90%, 95%, or 100% homology with the nucleotide sequences within 1-50 bases flanking either side of the cleavage site where the CasX cleaves the target nucleic acid sequence, facilitating insertion of the donor template sequence by HDR. In some embodiments, the donor template sequence comprises a non-homologous or a heterologous sequence flanked by two homologous arms, such that homology-directed repair between the target DNA region and the two flanking arm sequences results in insertion of the non-homologous or heterologous sequence at the target region, resulting in the knock-down or knock-out of the target gene, with a resulting reduction or elimination of expression of the gene product. In such knock-down cases, expression of the gene product is reduced by at least about 10%, at least about 20%, at least about 30%, at least about 40%, at least about 50%, at least about 60%, at least about 70%, at least about 80%, or at least about 90% in comparison to target nucleic acid that has not been modified. In other cases, an exogenous donor template may comprise a corrective sequence to be integrated, and is flanked by an upstream homologous arm and a downstream homologous arm, each having homology to the target nucleic acid sequence that is introduced into a cell. Use of such donor templates can result in expression of functional protein or expression of physiologically normal levels of functional protein after gene editing. In other cases, an exogenous donor template, which may comprise a mutation, a heterologous sequence, or a corrective sequence, is inserted between the ends generated by CasX cleavage by homology-independent targeted integration (HITI) mechanisms. The exogenous sequence inserted by HITI can be any length, for example, a relatively short sequence of between 1 and 50 nucleotides in length, or a longer sequence of about 50-1000 nucleotides in length. The lack of homology can be, for example, having no more than 20-50% sequence identity and/or lacking in specific hybridization at low stringency. In other cases, the lack of homology can further include a criterion of having no more than 5, 6, 7, 8, or 9 bp identity.

In some embodiments, the AAV vector comprises a donor template sequence wherein the sequence may comprise certain sequence differences as compared to the target nucleic acid sequence, e.g., restriction sites, nucleotide polymorphisms, selectable markers (e.g., drug resistance genes, fluorescent proteins, enzymes etc.), etc., which may be used to assess for successful insertion of the donor nucleic acid at the cleavage site or in some cases may be used for other purposes (e.g., to signify expression at the targeted genomic locus). Alternatively, these sequence differences may include flanking recombination sequences such as FLPs, loxP sequences, or the like, that can be activated at a later time for removal of the marker sequence. In some embodiments of the method, the donor polynucleotide comprises at least about 10, at least about 50, at least about 100, at least about 200, at least about 300, at least about 400, at least about 500, at least about 600, at least about 700 nucleotides. In other embodiments, the donor polynucleotide comprises at least about 10 to about 700 nucleotides, at least about 20 to about 600 nucleotides, at least about 40 to about 400 nucleotides. In some embodiments, the donor template is a single stranded DNA template or a single stranded RNA template.

In some cases, the methods do not comprise contacting a target nucleic acid sequence with a donor template, and the target nucleic acid sequence is modified such that nucleotides within the target nucleic acid sequence are deleted or inserted according to the cell's own repair pathways; for example, the cellular repair pathway can be NHEJ.

In other embodiments, the method provides an AAV encoding a CasX comprising one or more nuclear localization signal (NLS) of any or multiples of the embodiments described herein for targeting the CasX/gRNA to the nucleus of the cell. The NLS can be fused at or near the N-terminus, the C-terminus, or both of the CasX protein.

Introducing recombinant AAV vectors comprising sequences encoding the transgene components (e.g., the CasX, gRNA, promoters and accessory components and, optionally, the donor template sequences) of the disclosure into cells under in vitro conditions can occur in any suitable culture media and under any suitable culture conditions that promote the survival of the cells and production of the CasX:gRNA. Introducing recombinant AAV vectors into a target cell can be carried out in vivo, in vitro or ex vivo. In some embodiments of the method, vectors may be provided directly to a target host cell. For example, cells may be contacted with vectors having nucleic acids encoding the CasX and gRNA of any of the embodiments described herein and, optionally, having a donor template sequence such that the vectors are taken up by the cells. Methods for contacting cells with nucleic acid vectors that are plasmids include electroporation, calcium chloride transfection, microinjection, transduction and lipofection are well known in the art. In some embodiments, the AAV is selected from AAV1, AAV2, AAV3, AAV4, AAV5, AAV6, AAV7, AAV8, AAV9, AAV10, AAV11, AAV12, AAV 44.9, AAV-Rh74, or AAVRh10.

In some embodiments, the vector is administered to a subject at a therapeutically effective dose. In the foregoing, the subject is selected from the group consisting of mouse, rat, pig, non-human primate, and human. In particular embodiments, the subject is a human. In some embodiments of the methods, the vector is administered to a subject at a dose of at least about 1×10⁵ vector genomes/kg (vg), at least about 1×10⁶ vg/kg, at least about 1×10⁷ vg/kg, at least about 1×10⁸ vg/kg, at least about 1×10⁹ vg/kg, at least about 1×10¹⁰ vg/kg, at least about 1×10¹¹ vg/kg, at least about 1×10¹² vg/kg, at least about 1×10¹³ vg/kg, at least about 1×10¹⁴ vg/kg, at least about 1×10¹⁵ vg/kg, at least about 1×101⁶ vg/kg. The vector can be administered by a route of administration selected from the group consisting of subcutaneous, intradermal, intraneural, intranodal, intramedullary, intramuscular, intralumbar, intrathecal, subarachnoid, intraventricular, intracapsular, intravenous, intralymphatical, or intraperitoneal routes, wherein the administering method is injection, transfusion, or implantation.

AAV vectors used for providing the nucleic acids encoding gRNAs and the CasX proteins to a target host cell can include suitable promoters or other accessory elements for driving the expression, that is, transcriptional activation of the nucleic acid of interest. In some cases, the encoding nucleic acid of interest will be operably linked to a promoter. This may include ubiquitously acting promoters, for example, the CMV-beta-actin promoter, or inducible promoters, such as promoters that are active in particular cell populations or that respond to the presence of drugs such as tetracycline or kanamycin. By transcriptional activation, it is intended that transcription will be increased above basal levels in the target host cell comprising the vector by at least about 10-fold, by at least about 100-fold, more usually by at least about 1000-fold. In addition, vectors used for providing a nucleic acid encoding a gRNA and/or a CasX protein to a cell may include nucleic acid sequences that encode for selectable markers in the target cells, so as to identify cells that have taken up the CasX protein and/or the gRNA.

### VI. AAV Vectors

In other embodiments, the present disclosure provides recombinant AAV vectors comprising polynucleotides encoding the CasX proteins, the gRNAs, and the regulatory and accessory elements described herein.

In some embodiments, the disclosure provides a recombinant adeno-associated virus (rAAV) comprising: a) an AAV capsid protein, and b) the polynucleotide of any one of the embodiments described herein. In the foregoing embodiment, the polynucleotide can comprise sequences of components selected from: a first adeno-associated virus (AAV) inverted terminal repeat (ITR) sequence; a second AAV ITR sequence; a first promoter sequence of any of the embodiments described herein; a second promoter sequence of any of the embodiments described herein; a sequence encoding a CRISPR protein of any of the embodiments described herein; a sequence encoding at least a first guide RNA (gRNA) of any of the embodiments described herein; and one or more accessory element sequences of any of the embodiments described herein. In some embodiments, the polynucleotide comprises one or more sequences selected from the group of sequences set forth in Tables 8-10, 12, 13, and 17-22 and 24-27, or a sequence having at least 85%, at least 90%, at least 95%, at least 95%, at least 96%, at least 97%, at least 98%, or at least 99% identity thereto. In another embodiment, the polynucleotide comprises a sequence selected from the group of sequences set forth in Tables 8-10, 12, 13, and 17-22 and 24-27. In some embodiments, the polynucleotide sequence differs from those set forth in Tables 8-10, 12, 13, and 17-22 and 24-26 only in the selection of the targeting sequences of the gRNA or gRNAs encoded by the polynucleotide, wherein the targeting sequence is a sequence having 15 to 30 nucleotides capable of hybridizing with the sequence of a target nucleic acid. In a particular embodiment, the targeting sequence of the polynucleotide is selected from the group consisting of the sequences set forth in Table 27. In some embodiments, the present disclosure provides a polynucleotide of any of the embodiments described herein, wherein the polynucleotide has the configuration of a construct of any one of FIG. 24, 33-35, or 42.

In some embodiments, the AAV capsid protein is derived from serotype AAV1, AAV2, AAV3, AAV4, AAV5, AAV6, AAV7, AAV8, AAV9, AAV10, AAV11, AAV12, AAV 44.9, AAV-Rh74, or AAVRh10. In some embodiments, the AAV capsid protein and the 5′ and 3′ ITR are derived from the same serotype of AAV. In other embodiments, the AAV capsid protein and the 5′ and 3′ ITR are derived from different serotypes of AAV. In a particular embodiment, the 5′ and 3′ ITR are derived from AAV1. In another particular embodiment, the 5′ and 3′ ITR are derived from AAV2. In some embodiments, the polynucleotides comprise sequences encoding the reference CasX of SEQ ID NOS: 1-3. In other embodiments, the polynucleotides comprise sequences encoding the CasX variants of any of the embodiments described herein, including the CasX protein variants of SEQ ID NOS: 49-160, 40208-40369 and 40828-40912 as set forth in Table 3, or sequences having at least about 50%, at least about 60%, at least about 70%, at least about 80%, at least about 90%, at least about 95%, at least about 96%, at least about 97%, at least about 98%, or at least about 99% sequence identity thereto. In some embodiments, the polynucleotides encode gRNA scaffold sequences selected from the group consisting of SEQ ID NOS: 2101-2285, 39981-40026, 40913-40958, and 41817 as set forth in Table 2, or sequences having at least about 50%, at least about 60%, at least about 70%, at least about 80%, at least about 90%, at least about 95%, at least about 95%, at least about 96%, at least about 97%, at least about 98%, at least about 99% sequence identity thereto. In some embodiments, the gRNA comprises a targeting sequence having 15 to 30 nucleotides that is complementary to, and therefore hybridizes with, the target nucleic acid in a cell, and is linked to the 3′ end of the gRNA scaffold sequence.

In other embodiments, the disclosure provides AAV systems comprising a donor template nucleic acid, wherein the donor template comprises a nucleotide sequence having homology to a target nucleic acid sequence. In some embodiments, the donor template is intended for gene editing and comprises all or at least a portion of a target gene wherein upon insertion of the donor template, the gene is either knocked down, knocked out, or the mutation is corrected. In some embodiments, the donor template comprises a sequence that encodes at least a portion of a target nucleic acid exon. In other embodiments, the donor template has a sequence that encodes at least a portion of a target nucleic acid intron. In other embodiments, the donor template has a sequence that encodes at least a portion of a target nucleic acid intron-exon junction. In still other cases, the donor template sequence of the AAV systems comprises one or more mutations relative to a target nucleic acid. In the foregoing embodiments, the donor template can range in size from 10-700 nucleotides. In some embodiments, the donor template is a single-stranded DNA template.

In other aspects, the disclosure relates to methods to produce polynucleotide sequences encoding the AAV vector of any of the embodiments described herein, as well as methods to express and recover the AAV. In general, the methods include producing a polynucleotide sequence coding for the components of the expression cassette plus the flanking ITRs of any of the embodiments described herein and incorporating the encoding gene into an expression vector appropriate for a host cell. For production of the AAV vector of any of the embodiments described herein, the methods include transforming an appropriate host cell with an expression vector comprising the encoding polynucleotide, together with and the Rep and Cap sequences provided in trans, and culturing the host cell under conditions causing or permitting the resulting AAV to be produced, which are recovered by methods described herein or by standard purification methods known in the art. Rep and Cap can be provided to the packaging host cell as plasmids. Alternatively, the host cell genome may comprise stably integrated Rep and Cap genes. Suitable packaging cell lines are known to one of ordinary skill in the art. See for example, www.cellbiolabs.com/aav-expression-and-packaging. Methods of purifying AAV produced by host cell lines will be known to one of ordinary skill in the art, and include, without limitation, affinity chromatography, gradient centrifugation, and ion exchange chromatography. Standard recombinant techniques in molecular biology are used, along with the methods of the Examples, to make the polynucleotides and AAV vectors of the present disclosure.

In accordance with the disclosure, nucleic acid sequences that encode the reference CasX, the CasX variants, or the gRNA of any of the embodiments described herein (or their complement) are used to generate recombinant DNA molecules that direct the expression in appropriate host cells. Several cloning strategies are suitable for performing the present disclosure, many of which are used to generate a construct that comprises a gene coding for a composition of the present disclosure, or its complement. In some embodiments, the cloning strategy is used to create a gene that encodes a construct that comprises nucleotides encoding the reference CasX, the CasX variants, or the gRNA that is used to transform a host cell for expression of the composition.

In some approaches, a construct is first prepared containing the DNA sequences encoding the components of the AAV vector and transgene. Exemplary methods for the preparation of such constructs are described in the Examples. The construct is then used to create an expression vector suitable for transforming a host packaging cell, such as a eukaryotic host cell for the expression and recovery of the AAV vector comprising the transgene. The eukaryotic host packaging cell can be selected from BHK cells, HEK293 cells, HEK293T cells, NS0 cells, SP2/0 cells, YO myeloma cells, P3X63 mouse myeloma cells, PER cells, PER.C6 cells, hybridoma cells, NIH3T3 cells, COS cells, HeLa cells, CHO cells, or other eukaryotic cells known in the art suitable for the production of recombinant AAV. A number of transfection techniques are generally known in the art; see, e.g., Sambrook et al. (1989) Molecular Cloning, a laboratory manual, Cold Spring Harbor Laboratories, New York. Particularly suitable transfection methods include calcium phosphate co-precipitation, direct microinjection into cultured cells, electroporation, liposome mediated gene transfer, lipid-mediated transduction, and nucleic acid delivery using high-velocity microprojectiles. Exemplary methods for the creation of expression vectors, the transformation of host cells and the expression and recovery of the nucleic acids and the AAV vectors are described in the Examples.

The gene encoding the AAV vector can be made in one or more steps, either fully synthetically or by synthesis combined with enzymatic processes, such as restriction enzyme-mediated cloning, PCR and overlap extension, including methods more fully described in the Examples. The methods disclosed herein can be used, for example, to ligate sequences of polynucleotides encoding the various components (e.g., ITRs, CasX and gRNA, promoters and accessory elements) of a desired sequence to create the expression vector.

In some embodiments, host cells transfected with the above-described AAV expression vectors are rendered capable of providing AAV helper functions in order to replicate and encapsidate the nucleotide sequences flanked by the AAV ITRs to produce rAAV viral particles. AAV helper functions are generally AAV-derived coding sequences which can be expressed to provide AAV gene products that, in turn, function in trans for productive AAV replication. AAV helper functions are used herein to complement necessary AAV functions that are missing from the AAV expression vectors. Thus, AAV helper functions include one, or both of the major AAV ORFs (open reading frames), encoding the rep and cap coding regions, or functional homologues thereof. Accessory functions can be introduced into and then expressed in host cells using methods known to those of skill in the art. Commonly, accessory functions are provided by infection of the host cells with an unrelated helper virus. In some embodiments, accessory functions are provided using an accessory function vector. Depending on the host/vector system utilized, any of a number of suitable transcription and translation control elements, including constitutive and inducible promoters, transcription enhancer elements, transcription terminators, etc., may be used in the expression vector.

In some embodiments, the nucleotide sequence encoding the components of the AAV vector is codon optimized. This type of optimization can entail a mutation of an encoding nucleotide sequence to mimic the codon preferences of the intended host organism or cell while encoding the same CasX protein or other protein component. Thus, the codons can be changed, but the encoded protein remains unchanged. For example, if the intended host cell was a human cell, a human codon-optimized CasX-encoding nucleotide sequence could be used. The gene design can be performed using algorithms that optimize codon usage and amino acid composition appropriate for the host cell utilized in the production of the AAV vector. In one method of the disclosure, a library of polynucleotides encoding the components of the constructs is created and then assembled, as described above. The resulting genes are then assembled and the resulting genes used to transform a host cell and produce and recover the AAV vector compositions for evaluation of its properties, as described herein. In some embodiments, as described more fully below, the nucleotide sequence encoding the components of the AAV vector are engineered to remove CpG dinucleotides in order to reduce the immunogenicity of the components, while retaining their functional characteristics.

In some embodiments, a nucleotide sequence encoding a gRNA is operably linked to a regulatory element. In some embodiments, a nucleotide sequence encoding a CasX protein is operably linked to a regulatory element. In other cases, the nucleotide encoding the CasX and gRNA are linked and are operably linked to a single regulatory element. Exemplary accessory elements include a transcription promoter, a transcription enhancer element, a transcription termination signal, internal ribosome entry site (IRES) or P2A peptide to permit translation of multiple genes from a single transcript, polyadenylation sequences to promote downstream transcriptional termination, sequences for optimization of initiation of translation, and translation termination sequences. In some cases, the promoter is a constitutively active promoter. In some cases, the promoter is a regulatable promoter. In some cases, the promoter is an inducible promoter. In some cases, the promoter is a tissue-specific promoter. In some cases, the promoter is a cell type-specific promoter. In some cases, the transcriptional accessory element (e.g., the promoter) is functional in a targeted cell type or targeted cell population. For example, in some cases, the transcriptional accessory element can be functional in eukaryotic cells, e.g., packaging host cells for the production of the AAV vector. In some cases, the accessory element is a transcription activator that works in concert with a promoter to initiate transcription. By transcriptional activation, it is intended that transcription will be increased above basal levels in the target cell by 10-fold, by 100-fold, more usually by 1000-old.

Non-limiting examples of eukaryotic promoters (promoters functional in a eukaryotic cell) include EF-1alpha, EF-1alpha core promoter, those from cytomegalovirus (CMV) immediate early, herpes simplex virus (HSV) thymidine kinase, early and late SV40, long terminal repeats (LTRs) from retrovirus, and mouse metallothionein-I. Further non-limiting examples of eukaryotic promoters include the CMV promoter full-length promoter, the minimal CMV promoter, the chicken β-actin promoter, the RSV promoter, the HIV-Ltr promoter, the hPGK promoter, the HSV TK promoter, the Mini-TK promoter, the human synapsin I promoter which confers neuron-specific expression, the Mecp2 promoter for selective expression in neurons, the minimal IL-2 promoter, the Rous sarcoma virus enhancer/promoter (RSV), the spleen focus-forming virus long terminal repeat (LTR) promoter, the SV40 enhancer, the TBG promoter from the human thyroxine-binding globulin gene (Liver specific), the PGK promoter, the human ubiquitin C promoter, the UCOE promoter (Promoter of HNRPA2B1-CBX3), the Histone H2 promoter, the Histone H3 promoter, the Ula1 small nuclear RNA promoter (226 nt), the U1b2 small nuclear RNA promoter (246 nt) 26, the TTR minimal enhancer/promoter, the b-kinesin promoter, the ROSA26 promoter and the glyceraldehyde 3-phosphate dehydrogenase (GAPDH) promoter. In some embodiments, the promoter operably linked to the sequence encoding the first and/or the second gRNA is U6 (Kunkel, G R et al. U6 small nuclear RNA is transcribed by RNA polymerase III. Proc Natl Acad Sci USA. 83(22):8575 (1986)).

Non-limiting examples of pol II promoters suitable for use in the AAV constructs of the disclosure include, but are not limited to polyubiquitin C (UBC), cytomegalovirus (CMV), simian virus 40 (SV40), chicken beta-Actin promoter and rabbit beta-Globin splice acceptor site fusion (CAG), chicken β-actin promoter with cytomegalovirus enhancer (CB7), PGK, Jens Tornoe (JeT), GUSB, CBA hybrid (CBh), elongation factor-1 alpha (EF-1alpha), beta-actin, Rous sarcoma virus (RSV), silencing-prone spleen focus forming virus (SFFV), CMVd1 promoter, truncated human CMV (tCMVd2), minimal CMV promoter, chicken β-actin promoter, chicken β-actin promoter with cytomegalovirus enhancer (CB7), HSV TK promoter, Mini-TK promoter, minimal IL-2 promoter, GRP94 promoter, Super Core Promoter 1, Super Core Promoter 2, MLC, MCK, GRK1 protein promoter, Rho promoter, CAR protein promoter, hSyn Promoter, U1A promoter, Ribsomal Rpl and Rps promoters (e.g., hRpl30 and hRps18), CMV53 promoter, minimal SV40 promoter, CMV53 promoter, SFCp promoter, pJB42CAT5 promoter, MLP promoter, EFS promoter, MeP426 promoter, MecP2 promoter, MHCK7 promoter, beta-glucuronidase (GUSB), CK7 promoter, and CK8e promoter. In some embodiments, an AAV construct of the disclosure comprises a pol II promoter comprising a sequence as set forth in Table 8, or a sequence having at least 85%, at least 90%, at least 95%, at least 95%, at least 96%, at least 97%, at least 98%, or at least 99% identity thereto. In a particular embodiment, the pol II promoter is EF-1alpha, wherein the promoter enhances transfection efficiency, the transgene transcription or expression of the CRISPR nuclease, the proportion of expression-positive clones and the copy number of the episomal vector in long-term culture. In another particular embodiment, the pol II promoter is JeT, wherein the promoter enhances transfection efficiency, the transgene transcription or expression of the CRISPR nuclease, the proportion of expression-positive clones and the copy number of the episomal vector in long-term culture. In some embodiments, the pol II promoter is a truncated version of the foregoing promoters. In some embodiments the pol II promoter in an AAV construct of the disclosure has less than about 400 nucleotides, less than about 350 nucleotides, less than about 300 nucleotides, less than about 200 nucleotides, less than about 150 nucleotides, less than about 100 nucleotides, less than about 80 nucleotides, or less than about 40 nucleotides. In some embodiments the pol II promoter in an AAV construct of the disclosure has between about 40 to about 585 nucleotides, between about 100 to about 400 nucleotides, or between about 150 to about 300 nucleotides. In some embodiments, the AAV constructs of the disclosure comprise polynucleic acids encoding the pol II promoters of any of the foregoing embodiments of the paragraph, as well as the promoters of Table 8, and can be, in some cases, configured in relation to the other components of the constructs as depicted in any one of FIG. 24, 33-35 or 42.

In some embodiments, an AAV construct of the disclosure comprises a pol II promoter with a linked intron, wherein the intron enhances the ability of the promoter to increase transfection efficiency, the transgene transcription or expression of the CRISPR nuclease, the proportion of expression-positive clones and the copy number of the episomal vector in long-term culture. Exemplary embodiments of such promoter-intron combinations are described in the Examples.

Non-limiting examples of pol III promoters suitable for use in the AAV constructs of the disclosure include, but are notlimited to U6, mini U6, 7SK, and H1 variants, BiH1 (Bidrectional H1 promoter), BiU6, Bi7SK, BiH1 (Bidirectional U6, 7SK, and H1 promoters), gorilla U6, rhesus U6, human 7SK, and human H1 promoters. In the foregoing embodiment, the pol III promoter enhances the transcription of the gRNA encoded by the AAV. In some embodiments, an AAV construct of the disclosure comprises a pol III promoter comprising a sequence as set forth in Table 9, or a sequence having at least 85%, at least 90%, at least 95%, at least 95%, at least 96%, at least 97%, at least 98%, or at least 99% identity thereto. In some embodiments, the pol III promoter is a truncated version of the foregoing promoters. In some embodiments the pol III promoter in an AAV construct of the disclosure has less than about 250 nucleotides, less than about 220 nucleotides, less than about 200 nucleotides, less than about 160 nucleotides, less than about 140 nucleotides, less than about 130 nucleotides, less than about 120 nucleotides, less than about 100 nucleotides, less than about 80 nucleotides, or less than about 70 nucleotides. In some embodiments the pol III promoter in an AAV construct of the disclosure has between about 70 to about 245 nucleotides, between about 100 to about 220 nucleotides, or between about 120 to about 160 nucleotides. In some embodiments, the AAV constructs of the disclosure comprise polynucleic acids encoding the pol III promoters of any of the foregoing embodiments of the paragraph, as well as the promoters of Table 9, and can be, in some cases, configured in relation to the other components of the constructs as depicted in any one of FIG. 24, 33-35 or 42.

Selection of the appropriate promoter is well within the level of ordinary skill in the art, as it relates to controlling expression, e.g., for modifying a gene or other target nucleic acid. The expression vector may also contain a ribosome binding site for translation initiation and a transcription terminator. The expression vector may also include appropriate sequences for amplifying expression. The expression vector may also include nucleotide sequences encoding protein tags (e.g., 6×His tag, hemagglutinin tag, fluorescent protein, etc.) that can be fused to the CasX protein, thus resulting in a chimeric CasX protein that are used for purification or detection.

In some embodiments, the present disclosure provides a polynucleotide sequence encoding a gRNA and/or a CasX protein that is operably linked to an inducible promoter, a constitutively active promoter, a spatially restricted promoter (i.e., transcriptional control element, enhancer, tissue specific promoter, cell type specific promoter, etc.), or a temporally restricted promoter.

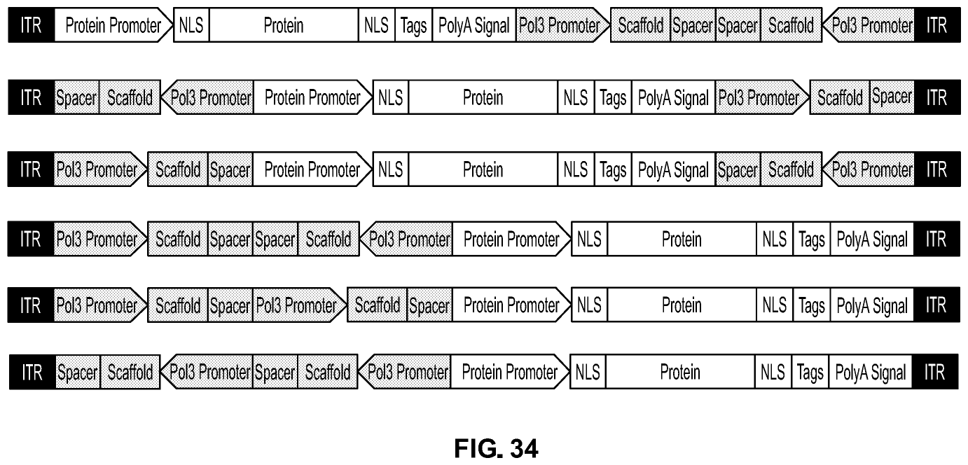

In certain embodiments, suitable promoters can be derived from viruses and can therefore be referred to as viral promoters, or they can be derived from any organism, including prokaryotic or eukaryotic organisms. Suitable promoters can be used to drive expression by any RNA polymerase (e.g., pol I, pol IL, pol III). Exemplary promoters include, but are not limited to the SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6), an enhanced U6 promoter, a human HI promoter (HI), a Pol II promoter, a 7SK promoter, tRNA promoters and the like. In some embodiments, the present disclosure provides a polynucleotide sequence wherein two gRNA of the transgene are operably linked to a single bidirectional promoter (e.g., bidrectional H1 promoter or bidirectional U6 promoter) placed between the two encoded gRNA sequences, wherein the promoter is capable of initiating transcription of both gRNA sequences. In other embodiments, the disclosure provides AAV constructs comprising promoters oriented in the reverse direction (i.e., 3′ to 5′). Exemplary reverse and bidirectional promoters are described in the Examples and Table 8 and are portrayed schematically in FIGS. 24 and 34.

In some embodiments, the present disclosure provides a polynucleotide sequence wherein one or more components of the transgene are operably linked to (under the control of) an inducible promoter operable in a eukaryotic cell. Examples of inducible promoters may include, but are not limited to, T7 RNA polymerase promoter, T3 RNA polymerase promoter, isopropyl-beta-D-thiogalactopyranoside (IPTG)-regulated promoter, lactose induced promoter, heat shock promoter, tetracycline-regulated promoter, kanamycin-regulated promoter, steroid-regulated promoter, metal-regulated promoter, estrogen receptor-regulated promoter, etc. Inducible promoters can therefore, in some embodiments, be regulated by molecules including, but not limited to, doxycycline, estrogen and/or an estrogen analog, IPTG, etc. Additional examples of inducible promoters include, without limitation, chemically/biochemically-regulated and physically-regulated promoters such as alcohol-regulated promoters, kanamycin-regulated promoters, tetracycline-regulated promoters (e.g., anhydrotetracycline (aTc)-responsive promoters and other tetracycline-responsive promoter systems, which include a tetracycline repressor protein (tetR), a tetracycline operator sequence (tetO) and a tetracycline transactivator fusion protein (tTA), steroid-regulated promoters (e.g., promoters based on the rat glucocorticoid receptor, human estrogen receptor, moth ecdysone receptors, and promoters from the steroid/retinoid/thyroid receptor superfamily), metal-regulated promoters (e.g., promoters derived from metallothionein (proteins that bind and sequester metal ions) genes from yeast, mouse and human), pathogenesis-regulated promoters (e.g., induced by salicylic acid, ethylene or benzothiadiazole (BTH)), temperature/heat-inducible promoters (e.g., heat shock promoters), and light-regulated promoters (e.g., light responsive promoters from plant cells).

In some cases, the promoter is a spatially restricted promoter (i.e., cell type specific promoter, tissue specific promoter, etc.) such that in a multi-cellular organism, the promoter is active (i.e., “ON”) in a subset of specific cells. Spatially restricted promoters may also be referred to as enhancers, transcriptional accessory elements, control sequences, etc. Any convenient spatially restricted promoter may be used as long as the promoter is functional in the targeted host cell (e.g., eukaryotic cell; prokaryotic cell).

In some cases, the promoter is a reversible promoter. Suitable reversible promoters, including reversible inducible promoters are known in the art. Such reversible promoters may be isolated and derived from many organisms, e.g., eukaryotes and prokaryotes. Modification of reversible promoters derived from a first organism for use in a second organism, e.g., a first prokaryote and a second a eukaryote, a first eukaryote and a second a prokaryote, etc., is well known in the art. Such reversible promoters, and systems based on such reversible promoters but also comprising additional control proteins, include, but are not limited to, alcohol regulated promoters (e.g., alcohol dehydrogenase I (alcA) gene promoter, promoters responsive to alcohol transactivator proteins (AlcR, etc.), tetracycline regulated promoters, (e.g., promoter systems including Tet Activators, TetON, TetOFF, etc.), steroid regulated promoters (e.g., rat glucocorticoid receptor promoter systems, human estrogen receptor promoter systems, retinoid promoter systems, thyroid promoter systems, ecdysone promoter systems, mifepristone promoter systems, etc.), metal regulated promoters (e.g., metallothionein promoter systems, etc.), pathogenesis-related regulated promoters (e.g., salicylic acid regulated promoters, ethylene regulated promoters, benzothiadiazole regulated promoters, etc.), temperature regulated promoters (e.g., heat shock inducible promoters (e.g., HSP-70, HSP-90, soybean heat shock promoter, etc.), light regulated promoters, synthetic inducible promoters, and the like.

Recombinant expression vectors of the disclosure can also comprise elements that facilitate robust expression components of the disclosure (e.g., the CasX or the gRNA). For example, recombinant expression vectors utilized in the AAV constructs of the disclosure can include one or more of a polyadenylation signal (poly(A)), an intronic sequence or a post-transcriptional accessory element (PTRE) such as a woodchuck hepatitis post-transcriptional accessory element (WPRE). Non-limiting examples of PTRE suitable for the AAV constructs of the disclosure include the sequences of Table 12, or a sequence having at least 85%, at least 90%, at least 95%, at least 95%, at least 96%, at least 97%, at least 98%, or at least 99% identity thereto. Exemplary poly(A) sequences suitable for inclusion in the expression vectors of the disclosure include hGH poly(A) signal (short), HSV TK poly(A) signal, synthetic polyadenylation signals, SV40 poly(A) signal, SV40 Late PolyA signal, β-globin poly(A) signal, β-globin poly(A) short, and the like. Non-limiting examples of poly(A) signals suitable for the AAV constructs of the disclosure include the sequences of Table 10, or a sequence having at least 85%, at least 90%, at least 95%, at least 95%, at least 96%, at least 97%, at least 98%, or at least 99% identity thereto. Non-limiting examples of introns suitable for the AAV constructs of the disclosure include the sequences of Table 17, or a sequence having at least 85%, at least 90%, at least 95%, at least 95%, at least 96%, at least 97%, at least 98%, or at least 99% identity thereto. A person of ordinary skill in the art will be able to select suitable elements to include in the recombinant expression vectors described herein.

The polynucleotides encoding the transgene components can be individually cloned into the AAV expression vector. In some embodiments, the polynucleotide is a recombinant expression vector that comprises a nucleotide sequence encoding a CasX protein. In other embodiments, the disclosure provides a recombinant expression vector comprising a polynucleotide sequence encoding a CasX protein and a nucleotide sequence encoding a first gRNA and, optionally, a second gRNA. In some cases, the nucleotide sequence encoding the CasX protein variant and/or the nucleotide sequence encoding the gRNA are each operably linked to a promoter that is operable in a cell type of choice. In other embodiments, the nucleotide sequence encoding the CasX protein variant and the nucleotide sequence encoding the gRNA are provided in separate vectors.

The nucleic acid sequences encoding the transgene components are inserted into the vector by a variety of procedures. In general, DNA is inserted into an appropriate restriction endonuclease site(s) using techniques known in the art. Vector components generally include, but are not limited to, one or more of a signal sequence, an origin of replication, one or more marker genes, an enhancer element, a promoter, and a transcription termination sequence. Construction of suitable vectors containing one or more of these components employs standard ligation techniques which are known to the skilled artisan. Such techniques are well known in the art and well described in the scientific and patent literature. Various vectors are publicly available.

The recombinant expression vectors can be delivered to the target host cells by a variety of methods, as described more fully, below, and in the Examples. Such methods include, e.g., viral infection, transfection, lipofection, electroporation, calcium phosphate precipitation, polyethyleneimine (PEI)-mediated transfection, DEAE-dextran mediated transfection, liposome-mediated transfection, particle gun technology, nucleofection, electroporation, cell squeezing, calcium phosphate precipitation, direct microinjection, nanoparticle-mediated nucleic acid delivery, and the like. A number of transfection techniques are generally known in the art; see, e.g., Sambrook et al. (1989) Molecular Cloning, a laboratory manual, Cold Spring Harbor Laboratories, New York. Packaging cells are typically used to form virus particles; such cells include HEK293 cells or HEK293T cells (and other cells known in the art), which package adenovirus.

In some embodiments, host cells transfected with the above-described AAV expression vectors are rendered capable of providing AAV helper functions in order to replicate and encapsidate the nucleotide sequences flanked by the AAV ITRs to produce rAAV viral particles. AAV helper functions are generally AAV-derived coding sequences which can be expressed to provide AAV gene products that, in turn, function in trans for productive AAV replication. In some embodiments, packaging cells are transfected with plasmids comprising AAV helper functions to complement necessary AAV functions that are missing from the AAV expression vectors. Thus, AAV helper function plasmids include one, or both of the major AAV ORFs (open reading frames), encoding the rep and cap coding regions, or functional homologues thereof, and the adenoviral helper genes comprising E2A, E4, and VA genes, operably linked to a promoter. Accessory functions can be introduced into and then expressed in host cells using methods known to those of skill in the art. Commonly, accessory functions are provided by infection of the host cells with an unrelated helper virus. In some embodiments, accessory functions are provided using an accessory function vector. Depending on the host/vector system utilized, any of a number of suitable transcription and translation accessory elements, including constitutive and inducible promoters, transcription enhancer elements, transcription terminators, etc., may be used in the expression vector.

### VII. Applications

The AAV systems provided herein are useful in methods for modifying the target nucleic acid sequence in various applications, including therapeutics, diagnostics, and research.

In the methods of modifying a target nucleic acid sequence in a cell described herein, the methods utilize any of the embodiments of the AAV systems described herein. In some cases, the methods knock-down the expression of the mutant gene product. In other cases, the methods knock-out the expression of the

mutant gene product. In still other cases, the methods result in the expression of functional protein of the gene product.

In some embodiments, the methods comprise contacting the target nucleic acid sequence with an AAV encoding a CasX protein and a guide nucleic acid comprising a targeting sequence, wherein said contacting results in modification of the target nucleic acid sequence by the CasX protein of the RNP. In some embodiments, the methods comprise introducing into a cell the AAV encoding the CasX protein and the gRNA, wherein the targeting sequence of the gRNA comprises a sequence complementary to a portion of the target nucleic acid, wherein the contacting results in the modification of the target nucleic acid of the RNP. In some embodiments, the encoded scaffold of the gRNA comprises a sequence selected from the group consisting of SEQ ID NOS: 2101-2285, 39981-40026, 40913-40958, and 41817 as set forth in Table 2, or a sequence having at least about 50%, at least about 60%, at least about 70%, at least about 80%, at least about 90%, at least about 95%, at least about 95%, at least about 96%, at least about 97%, at least about 98%, or at least about 99% sequence identity thereto, and the encoded CasX protein is a reference CasX protein SEQ ID NO: 1, SEQ ID NO: 2, or SEQ ID NO: 3 or a CasX variant comprising a sequence selected from the group consisting of SEQ ID NOS: 49-160, 40208-40369 and 40828-40912 as set forth in Table 3, or a sequence having at least about 50%, at least about 60%, at least about 70%, at least about 80%, at least about 90%, at least about 95%, at least about 95%, at least about 96%, at least about 97%, at least about 98%, at least about 99% sequence identity thereto.

In some embodiments, the modified target nucleic acid comprises a single-stranded break, resulting in a mutation, an insertion, or a deletion by the repair mechanisms of the cell. In other embodiments, the modified target nucleic acid comprises a double-stranded break, resulting in a mutation, an insertion, or a deletion by the repair mechanisms of the cell. For example, the CasX:gRNA system encoded by the AAV can introduce into the cell an indel, e.g., a frameshift mutation, at or near the initiation point of the gene. In other embodiments, the modified target nucleic acid of the cell has been modified by the insertion of the donor template wherein the gene comprising the target nucleic acid has been knocked down or knocked out.

In other embodiments, the method comprises contacting the target nucleic acid sequence with an AAV encoding a plurality (e.g., two or more) of gRNAs targeted to different or overlapping regions of the target nucleic acid with one or more mutations or duplications. In the foregoing, the resulting modification can be an insertion, deletion, substitution, duplication, or inversion of one or more nucleotides as compared to the target nucleic acid sequence.

### VIII. Therapeutic Methods

The present disclosure provides methods of treating a disease in a subject in need thereof. In some embodiments, the methods of the disclosure can prevent, treat and/or ameliorate a disease of a subject by the administering to the subject of an AAV composition of the disclosure. In some embodiments, the composition administered to the subject further comprises pharmaceutically acceptable carrier, diluent or excipient.

In some embodiments, the disclosure provides methods of treating a disease in a subject in need thereof comprising modifying a target nucleic acid in a cell of the subject, the modifying comprising administering to the subject a therapeutically effective dose of an AAV vector of any of the embodiments described herein wherein the targeting sequence of the encoded gRNA has a sequence that hybridizes with the target nucleic acid, resulting in the modification of the target nucleic acid by the CasX protein.

In other embodiments, the methods of treating a disease in a subject in need thereof comprise administering to the subject a therapeutically effective dose of an AAV vector of any of the embodiments described herein wherein the targeting sequence of the encoded gRNA has a sequence that hybridizes with the target nucleic acid and wherein the AAV further comprises a donor template comprises one or more mutations or a heterologous sequence that is inserted into or replaces the target nucleic acid sequence to knock-down or knock-out the gene comprising the target nucleic acid. In the foregoing, the insertion of the donor template serves to disrupt expression of the gene and the resulting gene product. In some embodiments of the foregoing methods, the donor DNA template ranges in size from 10-15,000 nucleotides. In other embodiments of the foregoing methods, the donor template ranges in size from 100-1,000 nucleotides. In some cases, the donor template is a single-stranded RNA or DNA template.

The modified cell of the treated subject can be a eukaryotic cell selected from the group consisting of a rodent cell, a mouse cell, a rat cell, a primate cell, a non-human primate cell, and a human cell. In some embodiments, the eukaryotic cell of the treated subject is a human cell.

In some embodiments, the method comprises administering to the subject the AAV vector of the embodiments described herein via an administration route selected from the group consisting of subcutaneous, intradermal, intraneural, intranodal, intramedullary, intramuscular, intralumbar, intrathecal, subarachnoid, intraventricular, intracapsular, intravenous, intralymphatical, intraocular or intraperitoneal routes, wherein the administering method is injection, transfusion, or implantation. In some embodiments of the methods of treating a disease in a subject, the subject is selected from the group consisting of mouse, rat, pig, non-human primate, and human. In a particular embodiment, the subject is a human.

In some embodiments of the method of treating a disease in a subject in need thereof, the AAV vector is administered at a dose of at least about 1×10⁵ vector genomes/kg (vg), at least about 1×10⁶ vg/kg, at least about 1×10⁷ vg/kg, at least about 1×10⁸ vg/kg, at least about 1×10⁹ vg/kg, at least about 1×10¹⁰ vg/kg, at least about 1×10¹¹ vg/kg, at least about 1×10¹² vg/kg, at least about 1×10¹³ vg/kg, at least about 1×10¹⁴ vg/kg, at least about 1×10¹⁵ vg/kg, at least about 1×101⁶ vg/kg. In organ systems like the eye, the AAV vector is administered at a dose of at least about 1×10⁵ vector genomes (vg), at least about 1×10⁶ vg, at least about 1×10⁷ vg, at least about 1×10⁸ vg, at least about 1×10⁹ vg, at least about 1×10¹⁰ vg, at least about 1×10¹¹ vg, at least about 1×10¹² vg, at least about 1×10¹³ vg, at least about 1×10¹⁴ vg, at least about 1×10¹⁵ vg, at least about 1×101⁶ vg.

A number of therapeutic strategies have been used to design the compositions for use in the methods of treatment of a subject with a disease. In some embodiments, the invention provides a method of treatment of a subject having a disease, the method comprising administering to the subject an AAV vector of any of the embodiments disclosed herein according to a treatment regimen comprising one or more consecutive doses using a therapeutically effective dose. In some embodiments of the treatment regimen, the therapeutically effective dose of the AAV vector is administered as a single dose. In other embodiments of the treatment regimen, the therapeutically effective dose is administered to the subject as two or more doses over a period of at least two weeks, or at least one month, or at least two months, or at least three months, or at least four months, or at least five months, or at least six months. In some embodiments of the treatment regiment, the effective doses are administered by a route selected from the group consisting of subcutaneous, intradermal, intraneural, intranodal, intramedullary, intramuscular, intralumbar, intrathecal, subarachnoid, intraventricular, intracapsular, intravenous, intralymphatical, intraocular, subretinal, intravitreal, or intraperitoneal routes, wherein the administering method is injection, transfusion, or implantation.

In some embodiments, the administering of the therapeutically effective amount of an AAV vector to knock down or knock out expression of a gene having one or more mutations leads to the prevention or amelioration of the underlying disease such that an improvement is observed in the subject, notwithstanding that the subject may still be afflicted with the underlying disease. In some embodiments, the administration of the therapeutically effective amount of the AAV vector leads to an improvement in at least one clinically-relevant parameter for the disease. In some embodiments of the method of treatment, the subject is selected from mouse, rat, pig, dog, non-human primate, and human.

In some embodiments, the disclosure provides compositions of any of the AAV embodiments described herein for use as a medicament for the treatment of a human in need thereof. In some embodiments, the medicament is administered to the subject according to a treatment regimen comprising one or more consecutive doses using a therapeutically effective dose.

### IX. AAV Engineered to Reduce Immunogenicity Retain Editing Properties

AAV-associated pathogen associated molecular patterns (PAMPs) that contribute to immune responses in mammalians hosts include: i) ligands present on rAAV viral capsids that bind toll-like receptor 2 (TLR2), a cell-surface PRR on non-parenchymal cells in the liver; and ii) unmethylated CpG dinucleotides in viral DNA that bind TLR9, an endosomal PRR in plasmacytoid dendritic cells (pDCs) and B cells (Faust, S M, et al. CpG-depleted adeno-associated virus vectors evade immune detection. J. Clinical Invest. 123:2294 (2013)). In particular, CpG dinucleotide motifs (CpG PAMPs) in AAV vectors are immunostimulatory because of their high degree of hypomethylation, relative to mammalian CpG motifs, which have a high degree of methylation. Accordingly, reducing the frequency of unmethylated CpGs in AAV vector genomes to a level below the threshold that activates human TLR9 is expected to reduce the immune response to exogenously administered AAV-based biologics. Similarly, methylation of CpG PAMPs in AAV constructs is similarly expected to reduce the immune response to AAV-based biologics.

In some embodiments, the present disclosure provides AAV vectors wherein one or more components of the transgene are codon-optimized for depletion of CpG dinucleotides by the substitution of homologous nucleotide sequences from mammalian species, wherein the one or more components substantially retain their functional properties upon expression in a transduced cell; e.g., ability to drive expression of the CRISPR nuclease, ability to drive expression of the gRNA, enhance the expression of the CRISPR nuclease and/or the gRNA, and enhanced ability to edit a target nucleic acid sequence. In some embodiments, the present disclosure provides AAV vectors wherein one or more AAV transgene component sequences selected from the group consisting of 5′ ITR, 3′ ITR, pol III promoter, pol II promoter, encoding sequence for CRISPR nuclease, encoding sequence for gRNA, accessory element, and poly(A) are codon-optimized for depletion of all or a portion of the CpG dinucleotides, wherein the resulting AAV vector transgene is substantially devoid of CpG dinucleotides. In some embodiments, the present disclosure provides AAV vectors wherein one or more AAV transgene component sequences selected from the group consisting of 5′ ITR, 3′ ITR, pol III promoter, pol II promoter, encoding sequence for a CRISPR nuclease, encoding sequence for gRNA, poly(A), and accessory element comprise less than about 10%, less than about 5%, or less than about 1% CpG dinucleotides. In some embodiments, the present disclosure provides AAV vectors wherein one or more AAV transgene component sequences selected from the group consisting of 5′ ITR, 3 ITR, pol III promoter, pol II promoter, encoding sequence for the CRISPR nuclease, encoding sequence for the gRNA, and poly(A) are devoid of CpG dinucleotides. In some embodiments, the present disclosure provides AAV vectors wherein the transgene comprises less than about 10%, less than about 5%, or less than about 1% CpG dinucleotides. In some embodiments, the present disclosure provides AAV vectors wherein the one or more AAV component sequences codon-optimized for depletion of CpG dinucleotides are selected from the group of sequences consisting of SEQ ID NOS: 41045-41055, as set forth in Table 25, or a sequence having at least about 80%, at least about 90%, at least about 95%, at least about 96%, at least about 97%, at least about 98%, or at least about 99% sequence identity thereto. In some embodiments, the disclosure provides AAV vectors having one or more components of the transgene codon-optimized for depletion of CpG dinucleotides, wherein the expressed CRISPR nuclease and gRNA retain at least about 60%, at least about 70%, at least about 80%, or at least about 90% of the editing potential for a target nucleic acid compared to an AAV vector wherein the transgene has not been codon-optimized for depletion of CpG dinucleotides, when assayed in an in vitro assay under comparable conditions. In a particular embodiment, the present disclosure provides AAV vectors wherein the one or more AAV component sequences codon-optimized for depletion of CpG dinucleotides that retain editing potential are selected from the group of sequences consisting of SEQ ID NOS: 41045-41055, as set forth in Table 25, or a sequence having at least about 80%, at least about 90%, at least about 95%, at least about 96%, at least about 97%, at least about 98%, or at least about 99% sequence identity thereto.

The embodiments of the AAV vector comprising the one or more components of the transgene codon-optimized for depletion of CpG dinucleotides have, as an improved characteristic, a lower potential for inducing an immune response, either in vivo (when administered to a subject) or in in vitro mammalian cell assays designed to detect markers of an inflammatory response. In some embodiments, the administration of a therapeutically effective dose of the AAV vector comprising the one or more components of the transgene codon-optimized for depletion of CpG dinucleotides to a subject results in a reduced immune response compared to the immune response of a comparable AAV vector wherein the transgene has not been codon-optimized for depletion of CpG dinucleotides, wherein the reduced response is determined by the measurement of one or more parameters such as production of antibodies or a delayed-type hypersensitivity to an AAV component, or the production of inflammatory cytokines and markers, such as, but not limited to TLR9, interleukin-1 (IL-1), IL-6, IL-12, IL-18, tumor necrosis factor alpha (TNF-α), interferon gamma (IFNγ), and granulocyte-macrophage colony stimulating factor (GM-CSF). In some embodiments, the AAV vector comprising the one or more components of the transgene that are substantially devoid of CpG dinucleotides elicits reduced production of one or more inflammatory markers selected from the group consisting of TLR9, interleukin-1 (IL-1), IL-6, IL-12, IL-18, tumor necrosis factor alpha (TNF-α), interferon gamma (IFNγ), and granulocyte-macrophage colony stimulating factor (GM-CSF) of at least about 10%, at least about 20%, at least about 30%, at least about 40%, at least about 50%, at least about 60%, at least about 80%, or at least about 90% compared to the comparable AAV that is not CpG depleted, when assayed in a cell-based vitro assay using cells known in the art appropriate for such assays; e.g., monocytes, macrophages, T-cells, B-cells, etc. In a particular embodiment, the AAV vector comprising the one or more components of the transgene codon-optimized for depletion of CpG dinucleotides exhibits a reduced activation of TLR9 in hNPCs in an in vitro assay of at least about 10%, at least about 20%, at least about 30%, at least about 40%, at least about 50%, at least about 60%, at least about 80%, or at least about 90% compared to the comparable AAV that is not CpG depleted.

### X. Kits and Articles of Manufacture

In other embodiments, provided herein are kits comprising an AAV vector of any of the embodiments of the disclosure, and a suitable container (for example a tube, vial or plate).

In some embodiments, the kit further comprises a buffer, a nuclease inhibitor, a protease inhibitor, a liposome, a therapeutic agent, a label, a label visualization reagent, or any combination of the foregoing. In some embodiments, the kit further comprises a pharmaceutically acceptable carrier, diluent or excipient.

In some embodiments, the kit comprises appropriate control compositions for gene modifying applications, and instructions for use.

### XI. Enumerated Embodiments

The following sets of enumerated embodiments are included for illustrative purposes and are not intend to limit the scope of the invention.

### Set I:

The embodiments of Set I refer to tables provided in U.S. provisional application 63/123,112 and to sequence listing submitted with U.S. provisional application 63/123,112 on Dec. 9, 2020.

Embodiment I-1. A polynucleotide, comprising
a. a first adeno-associated virus (AAV) inverted terminal repeat (ITR) sequence;
b. a second AAV ITR sequence;
c. a first promoter sequence;
d. a sequence encoding a CRISPR protein;
e. a sequence encoding at least a first guide RNA (gRNA); and, optionally,
f. at least one accessory element sequence.

Embodiment I-2. The polynucleotide of embodiment I-1, wherein the CRISPR protein sequence and the sequence encoding the at least first gRNA are less than about 3100, less than about 3090, less than about 3080, less than about 3070, less than about 3060, less than about 3050, or less than about 3040 nucleotides in length.

Embodiment I-3. The polynucleotide of embodiment I-1 or I-2, wherein the sequences of the first promoter and the at least one accessory element have greater than at least about 1300, at least about 1350, at least about 1360, at least about 1370, at least about 1380, at least about 1390, at least about 1400, at least about 1500, at least about 1600 nucleotides, at least 1650, at least about 1700, at least about 1750, at least about 1800, at least about 1850, or at least about 1900 nucleotides in combined length.

Embodiment I-4. The polynucleotide of embodiment I-1 or I-2, wherein the sequences of the first promoter and the at least one accessory element have greater than 1314 nucleotides in combined length.

Embodiment I-5. The polynucleotide of embodiment I-1 or I-2, wherein the sequences of the first promoter and the at least one accessory element have greater than 1381 nucleotides in combined length.

Embodiment I-6. The polynucleotide of any one of the preceding embodiments, wherein the first promoter sequence and the sequence encoding the CRISPR protein are operably linked.

Embodiment I-7. The polynucleotide of any one of the preceding embodiments, wherein the sequences encoding the CRISPR protein and the at least first guide RNA are operably linked to the first promoter.

Embodiment I-8. The polynucleotide of any one of the preceding embodiments, wherein the at least one accessory element is operably linked to the CRISPR protein.

Embodiment I-9. The polynucleotide of any one of embodiments I-1 to I-6, further comprising a second promoter.

Embodiment I-10. The polynucleotide of embodiment I-9, wherein the second promoter sequence and the sequence encoding the gRNA are operably linked.

Embodiment I-11. The polynucleotide of embodiment I-9 or I-10, wherein the sequences of the first promoter, the second promoter and the at least one accessory element are greater than at least about 1300, at least about 1350, at least about 1360, at least about 1370, at least about 1380, at least about 1390, at least about 1400, at least about 1500, at least about 1600 nucleotides, at least 1650, at least about 1700, at least about 1750, at least about 1800, at least about 1850, or at least about 1900 nucleotides in combined length.

Embodiment I-12. The polynucleotide of embodiment I-9 or I-10, wherein the sequences of the first promoter, the second promoter, and the at least one accessory element are greater than 1314 nucleotides in combined length.

Embodiment I-13. The polynucleotide of embodiment I-9 or I-10, wherein the sequences of the first promoter, the second promoter, and the at least one accessory element are greater than 1381 nucleotides in combined length.

Embodiment I-14. The polynucleotide of any one of embodiments I-1 to I-13, comprising two or more accessory elements.

Embodiment I-15. The polynucleotide of embodiment I-14, wherein the sequences of the first promoter, the second promoter, and the two or more accessory elements are greater than at least about 1300, at least about 1350, at least about 1360, at least about 1370, at least about 1380, at least about 1390, at least about 1400, at least about 1500, at least about 1600 nucleotides, at least 1650, at least about 1700, at least about 1750, at least about 1800, at least about 1850, or at least about 1900 nucleotides in combined length.

Embodiment I-16. The polynucleotide of embodiment I-14, wherein the sequences of the first promoter, the second promoter, and the two or more accessory elements are greater than 1314 nucleotides in combined length.

Embodiment I-17. The polynucleotide of embodiment I-14, wherein the sequences of the first promoter, the second promoter, and the two or more accessory elements are greater than 1381 nucleotides in combined length.

Embodiment I-18. The polynucleotide of any one of embodiments I-1 to I-17, wherein the polynucleotide comprises a second promoter, wherein at least 25%, 26%, 27%, 28%, 29%, 30%, 31%, 32%, 33%, 34%, or at least 35% or more of the length of the polynucleotide sequence comprises the sequences of the first and second promoters and the at least one accessory element in combined length.

Embodiment I-19. The polynucleotide of any one of the preceding embodiments, wherein the at least one accessory element is selected from the group consisting of a poly(A) signal, a gene enhancer element, an intron, a posttranscriptional regulatory element, a nuclear localization signal (NLS), a deaminase, a DNA glycosylase inhibitor, a third promoter, a second guide RNA, a stimulator of CRISPR-mediated homology-directed repair, an activator or repressor of transcription, and a self-cleaving sequence.

Embodiment I-20. The polynucleotide of any one of the preceding embodiments, wherein the accessory element(s) enhance the expression, binding, activity, or performance of the CRISPR protein as compared to the CRISPR protein in the absence of said accessory element.

Embodiment I-21. The polynucleotide of embodiment I-20, wherein the enhanced performance is an increase in editing of a target nucleic acid in an in vitro assay of at least about 10%, at least about 20%, at least about 30%, at least about 40%, at least about 50%, at least about 60%, at least about 70%, at least about 80%, at least about 90%, at least about 100%, at least about 1500%, at least about 200%, or at least about 300%.

Embodiment I-22. The polynucleotide of any one of the preceding embodiments, wherein the CRISPR protein is a Class 2 CRISPR protein.

Embodiment I-23. The polynucleotide of embodiment I-22, wherein the CRISPR protein is a Class 2, Type V CRISPR protein.

Embodiment I-24. The polynucleotide of embodiment I-23, wherein the Class 2, Type V CRISPR protein is a CasX.

Embodiment I-25. The polynucleotide of embodiment I-24, wherein the CasX comprises a sequence selected from the group consisting of SEQ ID NOS: 1-3 and 49-160 as set forth in Table 3, or a sequence having at least 85%, at least 90%, at least 95%, at least 95%, at least 96%, at least 97%, at least 98%, or at least 99% identity thereto.

Embodiment I-26. The polynucleotide of embodiment I-24, wherein the CasX comprises a sequence selected from the group consisting of the sequences of SEQ ID NOS: 1-3 and 49-160 as set forth in Table 3.

Embodiment I-27. The polynucleotide of any one of the preceding embodiments, wherein the first gRNA comprises a sequence selected from the group of sequences of SEQ ID NOS: 2101-2285 as set forth in Table 2, or a sequence having at least 85%, at least 90%, at least 95%, at least 95%, at least 96%, at least 97%, at least 98% identity thereto.

Embodiment I-28. The polynucleotide of any one of the preceding embodiments, wherein the first gRNA comprises a sequence selected from the group of sequences of SEQ ID NOS: 2101-2285 as set forth in Table 2.

Embodiment I-29. The polynucleotide of embodiment I-28, wherein the first gRNA comprises a targeting sequence complementary to a target nucleic acid sequence, wherein the targeting sequence has at least 15 to 20 nucleotides.

Embodiment I-30. The polynucleotide of any one of embodiments I-19 to I-29, wherein the second gRNA comprises a sequence selected from the sequences of SEQ ID NOS: 2101-2285 as set forth in Table 2.

Embodiment I-31. The polynucleotide of embodiment I-30, wherein the second gRNA comprises a targeting sequence complementary to a target nucleic acid sequence different than the target nucleic acid of embodiment I-28, wherein the targeting sequence has at least 15 to 20 nucleotides.

Embodiment I-32. The polynucleotide of any one of the preceding embodiments, comprising a sequence of Tables 4, 5, 6, 7, 9, 10, and 12, or a sequence having at least 85%, at least 90%, at least 95%, at least 95%, at least 96%, at least 97%, at least 98%, or at least 99% identity thereto.

Embodiment I-33. The polynucleotide of any one of embodiments I-1 to I-31, comprising a sequence of Tables 4, 5, 6, 7, 9, 10, and 12.

Embodiment I-34. The polynucleotide of any one of the preceding embodiments, wherein the accessory element is a post-transcriptional regulatory element (PTRE) selected from the group consisting of cytomegalovirus immediate/early intronA, hepatitis B virus PRE (HPRE), Woodchuck Hepatitis virus PRE (WPRE), and 5′ untranslated region (UTR) of human heat shock protein 70 mRNA (Hsp70).

Embodiment I-35. The polynucleotide of any one of the preceding embodiments, wherein the first promoter sequence has at least about 200, at least about 300, at least about 400, at least about 500, at least about 600, at least about 700, or at least about 800 nucleotides.

Embodiment I-36. The polynucleotide of any one of embodiments I-9 to I-35, wherein the second promoter sequence has at least about 200, at least about 300, at least about 400, at least about 500, at least about 600, at least about 700, or at least about 800 nucleotides.

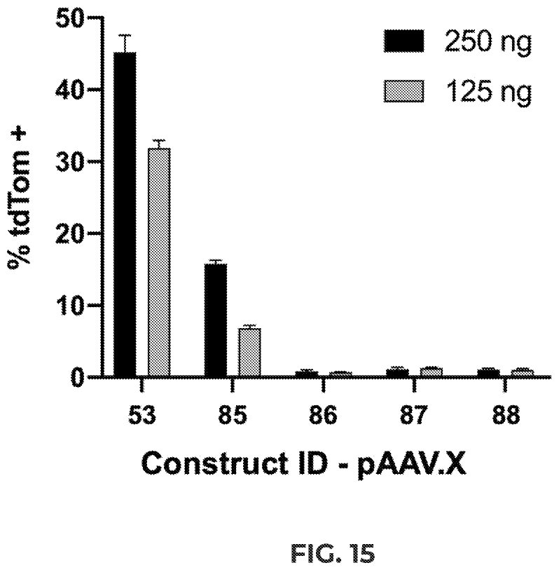

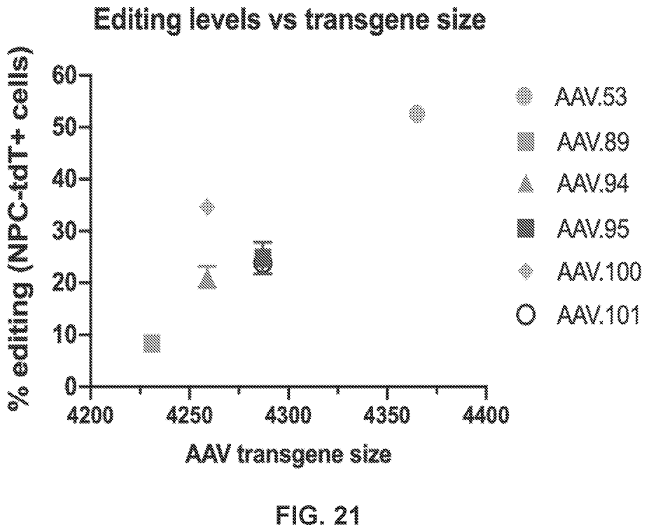

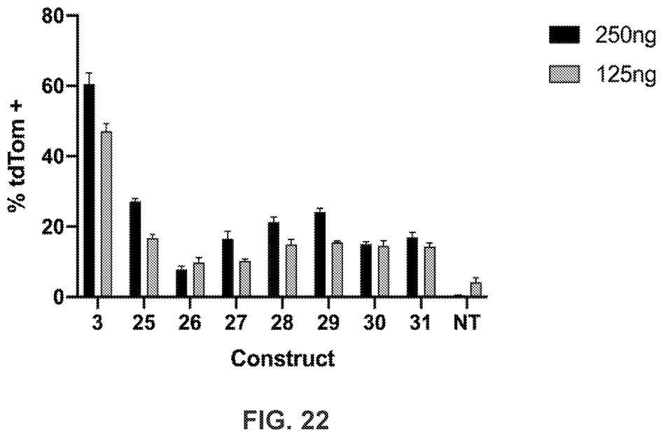

Embodiment I-37. The polynucleotide of any one of the preceding embodiments, wherein the polynucleotide has the configuration of a construct of FIG. 15, FIG. 21, or FIG. 22.

Embodiment I-38. The polynucleotide of any one of the preceding embodiments, wherein the 5′ and 3′ ITRs are derived from serotype AAV1, AAV2, AAV3, AAV4, AAV5, AAV6, AAV7, AAV8, AAV9, AAV10, AAV11, AAV12, AAV 44.9, AAV-Rh74, or AAVRh10.

Embodiment I-39. A recombinant adeno-associated virus (rAAV) comprising: a) an AAV capsid protein, and b) the polynucleotide of any one of embodiments I-1 to I-38.

Embodiment I-40. The rAAV of embodiment I-39, wherein the AAV capsid protein is derived from serotype AAV1, AAV2, AAV3, AAV4, AAV5, AAV6, AAV7, AAV8, AAV9, AAV10, AAV11, AAV12, AAV 44.9, AAV-Rh74, or AAVRh10.

Embodiment I-41. The rAAV of embodiment I-40, wherein the AAV capsid protein and the 5′ and 3′ ITR are derived from the same serotype of AAV.

Embodiment I-42. The rAAV of embodiment I-40, wherein the AAV capsid protein and the 5′ and 3′ ITR are derived from different serotypes of AAV.

Embodiment I-43. A pharmaceutical composition, comprising the rAAV of any one of embodiments I-39 to I-42 and a pharmaceutically acceptable carrier, diluent or excipient.

Embodiment I-44. A method for modifying a target nucleic acid in a population of mammalian cells, comprising contacting a plurality of the cells with an effective amount of the rAAV of any one of embodiments I-39 to I-42 or the pharmaceutical composition of embodiment I-43, wherein the target nucleic acid of the cells targeted by the gRNA is modified by the CRISPR protein.

Embodiment I-45. The method according to embodiment I-44, wherein the modifying comprises introducing an insertion, deletion, substitution, duplication, or inversion of one or more nucleotides in the target nucleic acid of the cells of the population.

Embodiment I-46. A method of making an rAAV vector, comprising:
i) providing a population of cells; and
ii) transfecting the population of cells with a vector comprising the polynucleotide of any one of embodiments I-1 to I-38.

Embodiment I-47. The method of embodiment I-46, wherein the population of cells express an AAV rep gene and AAV cap gene.

Embodiment I-48. The method of embodiment I-46, the method further comprising transfecting the cells with one or more vectors encoding an AAV rep gene and an AAV cap gene.

Embodiment I-49. The method of any one of embodiments I-46 to I-48, the method further comprising recovering the rAAV vector.

### Set II:

The embodiments of Set II refer to tables provided in U.S. provisional application 63/235,638 and to sequence listing submitted with U.S. provisional application 63/235,638 on Aug. 20, 2021.

Embodiment II-1. A polynucleotide, comprising
a. a first adeno-associated virus (AAV) inverted terminal repeat (ITR) sequence;
b. a second AAV ITR sequence;
c. a first promoter sequence;
d. a sequence encoding a CRISPR protein;
e. a sequence encoding at least a first guide RNA (gRNA); and,
f. optionally, at least one accessory element sequence.

Embodiment II-2. The polynucleotide of embodiment II-1, wherein the sequence encoding the CRISPR protein and the sequence encoding the at least first gRNA are less than about 3100, less than about 3090, less than about 3080, less than about 3070, less than about 3060, less than about 3050, or less than about 3040 nucleotides in length.

Embodiment II-3. The polynucleotide of embodiment II-1 or II-2, wherein the sequences of the first promoter and the at least one accessory element have greater than at least about 1300, at least about 1350, at least about 1360, at least about 1370, at least about 1380, at least about 1390, at least about 1400, at least about 1500, at least about 1600 nucleotides, at least 1650, at least about 1700, at least about 1750, at least about 1800, at least about 1850, or at least about 1900 nucleotides in combined length.

Embodiment II-4. The polynucleotide of embodiment II-1 or II-2, wherein the sequences of the first promoter and the at least one accessory element have greater than 1314 nucleotides in combined length.

Embodiment II-5. The polynucleotide of embodiment II-1 or II-2, wherein the sequences of the first promoter and the at least one accessory element have greater than 1381 nucleotides in combined length.

Embodiment II-6. The polynucleotide of any one of the preceding embodiments, wherein the first promoter sequence and the sequence encoding the CRISPR protein are operably linked.

Embodiment II-7. The polynucleotide of embodiment II-6, wherein the first promoter is a pol II promoter.

Embodiment II-8. The polynucleotide of embodiment II-6 or II-7, wherein the promoter is selected from the group consisting of polyubiquitin C (UBC), cytomegalovirus (CMV), simian virus 40 (SV40), chicken beta-Actin promoter and rabbit beta-Globin splice acceptor site fusion (CAG), chicken β-actin promoter with cytomegalovirus enhancer (CB7), PGK, Jens Tornoe (JeT), GUSB, CBA hybrid (CBh), elongation factor-1 alpha (EF-1alpha), beta-actin, Rous sarcoma virus (RSV), silencing-prone spleen focus forming virus (SFFV), CMVd1 promoter, truncated human CMV (tCMVd2), minimal CMV promoter, chicken β-actin promoter, HSV TK promoter, Mini-TK promoter, minimal IL-2 promoter, GRP94 promoter, Super Core Promoter 1, Super Core Promoter 2, MLC, MCK, GRK1 protein promoter, Rho promoter, CAR protein promoter, hSyn Promoter, U1A promoter, Ribsomal Rpl and Rps promoters (e.g., hRpl30 and hRps18), CMV53 promoter, minimal SV40 promoter, CMV53 promoter, SFCp promoter, pJB42CAT5 promoter, MLP promoter, EFS promoter, MeP426 promoter, MecP2 promoter, MHCK7 promoter, beta-glucuronidase (GUSB), CK7 promoter, and CK8e promoter.

Embodiment II-9. The polynucleotide of embodiment II-8, wherein the promoter is a truncated variant of the UBC, CMV, SV40, CAG, CB7, PGK, JeT, GUSB, CB, EF-1alpha, beta-actin, RSV, SFFV, CMVd1, tCMVd2, minimal CMV, chicken β-actin, HSV TK, Mini-TK, minimal IL-2, GRP94, Super Core Promoter 1, Super Core Promoter 2, MLC, MCK, GRK1 protein Rho, CAR protein, hSyn, U1A r, Ribsomal Rpl, and Rps (e.g., hRpl30 and hRps18), CMV53, SV40 promoter, CMV53, SFCp, pJB42CAT5, MLP, EFS, MeP426, MecP2, MHCK7, (GUSB, CK7, or CK8e promoter.

Embodiment II-10. The polynucleotide of embodiment II-8 or II-9, wherein the promoter has less than about 400 nucleotides, less than about 350 nucleotides, less than about 300 nucleotides, less than about 200 nucleotides, less than about 150 nucleotides, less than about 100 nucleotides, less than about 80 nucleotides, or less than about 40 nucleotides.

Embodiment II-11. The polynucleotide of embodiment II-8 or II-9, wherein the promoter has between about 40 to about 585 nucleotides, between about 100 to about 400 nucleotides, or between about 150 to about 300 nucleotides.

Embodiment II-12. The polynucleotide of any one of the preceding embodiments, wherein the promoter is selected from the group consisting of SEQ ID NOS: 40370-40400 as set forth in Table 4, or a sequence having at least 85%, at least 90%, at least 95%, at least 95%, at least 96%, at least 97%, at least 98%, or at least 99% identity thereto.

Embodiment II-13. The polynucleotide of any one of the preceding embodiments, wherein the at least one accessory element is operably linked to the CRISPR protein.

Embodiment II-14. The polynucleotide of any one of embodiments II-1 to II-6, further comprising a second promoter.

Embodiment II-15. The polynucleotide of embodiment II-14, wherein the second promoter sequence and the sequence encoding the gRNA are operably linked.

Embodiment II-16. The polynucleotide of embodiment II-14 or II-15, wherein the second promoter is a pol III promoter.

Embodiment II-17. The polynucleotide of any one of embodiments II-10 to II-12, wherein the second promoter is selected from the group consisting of U6, mini U61, mini U62, mini U63, BiH1 (Bidrectional H1 promoter), BiU6 (Bidirectional U6 promoter), gorilla U6, rhesus U6, human 7sk, and human H1 promoters.

Embodiment II-18. The polynucleotide of embodiment II-17, wherein the promoter is a truncated variant of the U6, mini U61, mini U62, mini U63, BiH1, BiU6, gorilla U6, rhesus U6, human 7sk, or human H1 promoter.

Embodiment II-19. The polynucleotide of embodiment II-17 or II-18, wherein the promoter has less than about 250 nucleotides, less than about 220 nucleotides, less than about 200 nucleotides, less than about 160 nucleotides, less than about 140 nucleotides, less than about 130 nucleotides, less than about 120 nucleotides, less than about 100 nucleotides, less than about 80 nucleotides, or less than about 70 nucleotides.

Embodiment II-20. The polynucleotide of embodiment II-17 or II-18, wherein the promoter has between about 70 to about 245 nucleotides, between about 100 to about 220 nucleotides, or between about 120 to about 160 nucleotides.

Embodiment II-21. The polynucleotide of any one of embodiments II-14 to II-20, wherein the promoter is selected from the group consisting SEQ ID NOS: 40401-40400 as set forth in Table 5, or a sequence having at least 85%, at least 90%, at least 95%, at least 95%, at least 96%, at least 97%, at least 98%, or at least 99% identity thereto.

Embodiment II-22. The polynucleotide of any one of embodiments II-14 to II-21, wherein the second promoter enhances transcription of the gRNA.

Embodiment II-23. The polynucleotide of any one of embodiments II-14 to II-22, wherein the sequences of the first promoter and the second promoter are greater than at least about 1300, at least about 1350, at least about 1360, at least about 1370, at least about 1380, at least about 1390, at least about 1400, at least about 1500, at least about 1600 nucleotides, at least 1650, at least about 1700, at least about 1750, at least about 1800, at least about 1850, or at least about 1900 nucleotides in combined length.

Embodiment II-24. The polynucleotide of any one of embodiments II-14 to II-23, wherein the sequences of the first promoter, the second promoter and the at least one accessory element are greater than at least about 1300, at least about 1350, at least about 1360, at least about 1370, at least about 1380, at least about 1390, at least about 1400, at least about 1500, at least about 1600 nucleotides, at least 1650, at least about 1700, at least about 1750, at least about 1800, at least about 1850, or at least about 1900 nucleotides in combined length.

Embodiment II-25. The polynucleotide of any one of embodiments II-14 to II-24, wherein the sequences of the first promoter, the second promoter, and the at least one accessory element are greater than 1314 nucleotides in combined length.

Embodiment II-26. The polynucleotide of any one of embodiments II-14 to II-24, wherein the sequences of the first promoter, the second promoter, and the at least one accessory element are greater than 1381 nucleotides in combined length.

Embodiment II-27. The polynucleotide of any one of the preceding embodiments, comprising two or more accessory elements.

Embodiment II-28. The polynucleotide of embodiment II-27, wherein the sequences of the first promoter, the second promoter, and the two or more accessory elements are greater than at least about 1300, at least about 1350, at least about 1360, at least about 1370, at least about 1380, at least about 1390, at least about 1400, at least about 1500, at least about 1600, at least 1650, at least about 1700, at least about 1750, at least about 1800, at least about 1850, or greater than at least about 1900 nucleotides in combined length.

Embodiment II-29. The polynucleotide of embodiment II-27, wherein the sequences of the first promoter, the second promoter, and the two or more accessory elements are greater than 1314 nucleotides in combined length.

Embodiment II-30. The polynucleotide of embodiment II-27, wherein the sequences of the first promoter, the second promoter, and the two or more accessory elements are greater than 1381 nucleotides in combined length.

Embodiment II-31. The polynucleotide of any one of embodiment II-14 to II-30, wherein at least 25%, 26%, 27%, 28%, 29%, 30%, 31%, 32%, 33%, 34%, or at least 35% or more of the length of the polynucleotide sequence comprises the sequences of the first and second promoters and the at least one accessory element in combined length.

Embodiment II-32. The polynucleotide of any one of the preceding embodiments, wherein the accessory elements are selected from the group consisting of a poly(A) signal, a gene enhancer element, an intron, a posttranscriptional regulatory element (PTRE), a nuclear localization signal (NLS), a deaminase, a DNA glycosylase inhibitor, a third promoter, a second guide RNA, a stimulator of CRISPR-mediated homology-directed repair, and an activator or repressor of transcription.

Embodiment II-33. The polynucleotide of any one of the preceding embodiments, wherein the accessory elements enhance the transcription, transcription termination, expression, binding, activity, or performance of the CRISPR protein as compared to an otherwise identical polynucleotide lacking said accessory elements.

Embodiment II-34. The polynucleotide of embodiment II-33, wherein the enhanced performance is an increase in editing of a target nucleic acid by the CRISPR protein in an in vitro assay of at least about 10%, at least about 20%, at least about 30%, at least about 40%, at least about 50%, at least about 60%, at least about 70%, at least about 80%, at least about 90%, at least about 100%, at least about 150%, at least about 200%, or at least about 300%.

Embodiment II-35. The polynucleotide of any one of the preceding embodiments, wherein the CRISPR protein is a Class 2 CRISPR protein.

Embodiment II-36. The polynucleotide of embodiment II-35, wherein the CRISPR protein is a Class 2, Type V CRISPR protein.

Embodiment II-37. The polynucleotide of embodiment II-36, wherein the Class 2, Type V CRISPR protein is a CasX.

Embodiment II-38. The polynucleotide of embodiment II-37, wherein the encoded CasX comprises a sequence selected from the group consisting of SEQ ID NOS: 1-3, 49-160, and 40208-40369 as set forth in Table 3, and SEQ ID NOS: 40808-40827, as set forth in Table 21, or a sequence having at least 85%, at least 90%, at least 95%, at least 95%, at least 96%, at least 97%, at least 98%, or at least 99% identity thereto.

Embodiment II-39. The polynucleotide of embodiment II-37, wherein the encoded CasX comprises a sequence selected from the group consisting of the sequences of SEQ ID NOS: 1-3, 49-160 and 40208-40369, as set forth in Table 3 and SEQ ID NOS: 40808-40827, as set forth in Table 21.

Embodiment II-40. The polynucleotide of any one of embodiments II-35 to II-39, wherein the polynucleotide encodes one or more NLS linked to the sequence encoding the CasX.

Embodiment II-41. The polynucleotide of embodiment II-40, wherein the sequences encoding the one or more NLS are positioned at or near the 5′ end of the sequence encoding the CasX protein.

Embodiment II-42. The polynucleotide of embodiment II-40 or II-41, wherein the sequences encoding the one or more NLS are positioned at or near at the 3′ end of the sequence encoding the CasX protein.

Embodiment II-43. The polynucleotide of embodiment II-41 or II-42, wherein the polynucleotide encodes at least two NLS, wherein the sequences encoding the at least two NLS are positioned at or near the 5′ and 3′ ends of the sequence encoding the CasX protein.

Embodiment II-44. The polynucleotide of any one of embodiments II-40 to II-43, wherein the one or more encoded NLS are selected from the group of sequences consisting of PKKKRKV (SEQ ID NO: 196), KRPAATKKAGQAKKKK (SEQ ID NO: 197), PAAKRVKLD (SEQ ID NO: 248), RQRRNELKRSP (SEQ ID NO: 161), NQSSNFGPMKGGNFGGRSSGPYGGGGQYFAKPRNQGGY (SEQ ID NO: 162), RMRIZFKNKGKDTAELRRRRVEVSVELRKAKKDEQILKRRNV (SEQ ID NO: 163), VSRKRPRP (SEQ ID NO: 164), PPKKARED (SEQ ID NO: 165), PQPKKKPL (SEQ ID NO: 166), SALIKKKKKMAP (SEQ ID NO: 167), DRLRR (SEQ ID NO: 168), PKQKKRK (SEQ ID NO: 169), RKLKKKIKKL (SEQ ID NO: 170), REKKKFLKRR (SEQ ID NO: 171), KRKGDEVDGVDEVAKKKSKK (SEQ ID NO: 172), RKCLQAGMNLEARKTKK (SEQ ID NO: 173), PRPRKIPR (SEQ ID NO: 174), PPRKKRTVV (SEQ ID NO: 175), NLSKKKKRKREK (SEQ ID NO: 176), RRPSRPFRKP (SEQ ID NO: 177), KRPRSPSS (SEQ ID NO: 178), KRGINDRNFWRGENERKTR (SEQ ID NO: 179), PRPPKMARYDN (SEQ ID NO: 180), KRSFSKAF (SEQ ID NO: 181), KLKIKRPVK (SEQ ID NO: 182), PKKKRKVPPPPAAKRVKLD (SEQ ID NO: 183), PKTRRRPRRSQRKRPPT (SEQ ID NO: 184), SRRRKANPTKLSENAKKLAKEVEN (SEQ ID NO: 185), KTRRRPRRSQRKRPPT (SEQ ID NO: 186), RRKKRRPRRKKRR (SEQ ID NO: 187), PKKKSRKPKKKSRK (SEQ ID NO: 188), HKKKHPDASVNFSEFSK (SEQ ID NO: 189), QRPGPYDRPQRPGPYDRP (SEQ ID NO: 190), LSPSLSPLLSPSLSPL (SEQ ID NO: 191), RGKGGKGLGKGGAKRHRK (SEQ ID NO: 192), PKRGRGRPKRGRGR (SEQ ID NO: 193), PKKKRKVPPPPKKKRKV (SEQ ID NO: 195), PAKRARRGYKC (SEQ ID NO: 40408), KLGPRKATGRW (SEQ ID NO: 40809), PRRKREE (SEQ ID NO: 40810), PYRGRKE (SEQ ID NO: 40811), PLRKRPRR (SEQ ID NO: 40812), PLRKRPRRGSPLRKRPRR (SEQ ID NO: 40813), PAAKRVKLDGGKRTADGSEFESPKKKRKV (SEQ ID NO: 40814), PAAKRVKLDGGKRTADGSEFESPKKKRKVGIHGVPAA (SEQ ID NO: 40815), PAAKRVKLDGGKRTADGSEFESPKKKRKVAEAAAKEAAAKEAAAKA (SEQ ID NO: 40816), PAAKRVKLDGGKRTADGSEFESPKKKRKVPG (SEQ ID NO: 40452), KRKGSPERGERKRHW, KRTADSQHSTPPKTKRKVEFEPKKKRKV (SEQ ID NO: 40817), and PKKKRKVGGSKRTADSQHSTPPKTKRKVEFEPKKKRKV (SEQ ID NO: 40818) wherein the one or more NLS are linked to the CasX variant or to adjacent NLS with a linker peptide wherein the linker peptide is selected from the group consisting of (G)n (SEQ ID NO: 40201), (GS)n (SEQ ID NO: 40202), (GSGGS)n (SEQ ID NO: 208), (GGSGGS)n (SEQ ID NO: 209), (GGGS)n (SEQ ID NO: 210), GGSG (SEQ ID NO: 211), GGSGG (SEQ ID NO: 212), GSGSG (SEQ ID NO: 213), GSGGG (SEQ ID NO: 214), GGGSG (SEQ ID NO: 215), GSSSG (SEQ ID NO: 216), GPGP (SEQ ID NO: 217), GGP, PPP, PPAPPA (SEQ ID NO: 218), PPPG (SEQ ID NO: 40207), PPPGPPP (SEQ ID NO: 219), PPP(GGGS)n (SEQ ID NO: 40203), (GGGS)nPPP (SEQ ID NO: 40204), AEAAAKEAAAKEAAAKA (SEQ ID NO: 40205), and TPPKTKRKVEFE (SEQ ID NO: 40206), where n is 1 to 5.

Embodiment II-45. The polynucleotide of any one of embodiments II-40 to II-44, wherein the one or more encoded NLS are selected from the group consisting of SEQ ID NOS: 40443-40501 as set forth in Table 11 and Table 12, or a sequence having at least 85%, at least 90%, at least 95%, at least 95%, at least 96%, at least 97%, at least 98% identity thereto.

Embodiment II-46. The polynucleotide of any one of embodiments II-40 to II-43, wherein the one or more encoded NLS are selected from the group of sequences consisting of SEQ ID NOS: 40443-40501 as set forth in Table 11 and Table 12.

Embodiment II-47. The polynucleotide of any one of the preceding embodiments, wherein the first gRNA comprises a sequence selected from the group consisting of SEQ ID NOS: 2101-2285, and 39981-40026, as set forth in Table 2, or a sequence having at least 85%, at least 90%, at least 95%, at least 95%, at least 96%, at least 97%, at least 98% identity thereto.

Embodiment II-48. The polynucleotide of any one of the preceding embodiments, wherein the first gRNA comprises a sequence selected from the group consisting of SEQ ID NOS: 2101-2285, and 39981-40026, as set forth in Table 2.

Embodiment II-49. The polynucleotide of embodiment II-48, wherein the first gRNA comprises a targeting sequence complementary to a target nucleic acid sequence, wherein the targeting sequence has at least 15 to 30 nucleotides.

Embodiment II-50. The polynucleotide of embodiment II-49, wherein the targeting sequence has 18, 19, or 20 nucleotides.

Embodiment II-51. The polynucleotide of any one of embodiments II-32 to II-50, wherein the second gRNA comprises a sequence selected from the group consisting of SEQ ID NOS: 2101-2285, and 39981-40026, as set forth in Table 2, or a sequence having at least 85%, at least 90%, at least 95%, at least 95%, at least 96%, at least 97%, at least 98% identity thereto.

Embodiment II-52. The polynucleotide of any one of embodiments II-32 to II-51, wherein the second gRNA comprises a sequence selected from the group consisting of SEQ ID NOS: 2101-2285, and 39981-40026, as set forth in Table 2.

Embodiment II-53. The polynucleotide of embodiment II-51 or II-52, wherein the second gRNA comprises a targeting sequence complementary to a target nucleic acid sequence different than the target nucleic acid of embodiment II-49 or II-50, wherein the targeting sequence has at least 15 to 30 nucleotides.

Embodiment II-54. The polynucleotide of embodiment II-53, wherein the targeting sequence has 18, 19, or 20 nucleotides.

Embodiment II-55. The polynucleotide of any one of the preceding embodiments, wherein the accessory element is a post-transcriptional regulatory element (PTRE) selected from the group consisting of cytomegalovirus immediate/early intronA, hepatitis B virus PRE (HPRE), Woodchuck Hepatitis virus PRE (WPRE), and 5′ untranslated region (UTR) of human heat shock protein 70 mRNA (Hsp70).

Embodiment II-56. The polynucleotide of any one of embodiments II-1 to II-55, wherein the accessory element is a PTRE selected from the group consisting SEQ ID NOS: 40431-40442 as set forth in Table 8, or a sequence having at least 85%, at least 90%, at least 95%, at least 95%, at least 96%, at least 97%, at least 98% identity thereto.

Embodiment II-57. The polynucleotide of any one of the preceding embodiments, wherein the 5′ and 3′ ITRs are derived from serotype AAV1, AAV2, AAV3, AAV4, AAV5, AAV6, AAV7, AAV8, AAV9, AAV10, AAV11, AAV12, AAV 44.9, AAV-Rh74, or AAVRh10.

Embodiment II-58. The polynucleotide of any one of the preceding embodiments, wherein the 5′ and 3′ ITRs are derived from serotype AAV2.

Embodiment II-59. The polynucleotide of any one of the preceding embodiments, comprising one or more sequences selected from the group consisting of the sequences of Tables 4, 5, 6, 8, 9, 13-16 and 20, or a sequence having at least 85%, at least 90%, at least 95%, at least 95%, at least 96%, at least 97%, at least 98%, or at least 99% identity thereto.

Embodiment II-60. The polynucleotide of any one of the preceding embodiments, comprising one or more sequences selected from the group consisting of the sequences of Tables 4, 5, 6, 8, 9, 13-16 and 20.

Embodiment II-61. The polynucleotide of any one of the preceding embodiments, wherein the polynucleotide has the configuration of a construct depicted in any one of FIG. 24, 33-35, or 42.

Embodiment II-62. A recombinant adeno-associated virus (rAAV) comprising: a) an AAV capsid protein, and b) the polynucleotide of any one of embodiments II-1 to II-58.

Embodiment II-63. The rAAV of embodiment II-62, wherein the AAV capsid protein is derived from serotype AAV1, AAV2, AAV3, AAV4, AAV5, AAV6, AAV7, AAV8, AAV9, AAV10, AAV11, AAV12, AAV 44.9, AAV-Rh74, or AAVRh10.

Embodiment II-64. The rAAV of embodiment II-63, wherein the AAV capsid protein and the 5′ and 3′ ITR are derived from the same serotype of AAV.

Embodiment II-65. The rAAV of embodiment II-63, wherein the AAV capsid protein and the 5′ and 3′ ITR are derived from different serotypes of AAV.

Embodiment II-66. The rAAV of embodiment II-65, wherein the 5′ and 3′ ITR are derived from AAV serotype 2.

Embodiment II-67. A pharmaceutical composition, comprising the rAAV of any one of embodiment II-62 and a pharmaceutically acceptable carrier, diluent or excipient.

Embodiment II-68. A method for modifying a target nucleic acid in a population of mammalian cells, comprising contacting a plurality of the cells with an effective amount of the rAAV of any one of embodiments II-62-66 or the pharmaceutical composition of embodiment II-67, wherein the target nucleic acid of the cells targeted by the gRNA is modified by the CRISPR protein.

Embodiment II-69. The method according to embodiment II-68, wherein the modifying comprises introducing an insertion, deletion, substitution, duplication, or inversion of one or more nucleotides in the target nucleic acid of the cells of the population.

Embodiment II-70. The method of embodiment II-68 or II-69, wherein the rAAV is administered to a subject at a dose of at least about 1×10⁸ vector genomes (vg), at least about 1×10⁵ vector genomes/kg (vg/kg), at least about 1×10⁶ vg/kg, at least about 1×10⁷ vg/kg, at least about 1×10⁸ vg/kg, at least about 1×10⁹ vg/kg, at least about 1×10¹⁰ vg/kg, at least about 1×10¹¹ vg/kg, at least about 1×10¹² vg/kg, at least about 1×10¹³ vg/kg, at least about 1×10¹⁴ vg/kg, at least about 1×10¹⁵ vg/kg, or at least about 1×10¹⁶ vg/kg.

Embodiment II-71. The method of embodiment II-68 or II-69, wherein the rAAV is administered to a subject at a dose of at least about 1×10⁵ vg/kg to about 1×10¹⁶ vg/kg, at least about 1×10⁶ vg/kg to about 1×10¹⁵ vg/kg, or at least about 1×10⁷ vg/kg to about 1×10¹⁴ vg/kg.

Embodiment II-72. The method of any one of embodiments 11-68 to II-71, wherein the rAAV is administered to the subject by a route of administration selected from subcutaneous, intradermal, intraneural, intranodal, intramedullary, intramuscular, intralumbar, intrathecal, subarachnoid, intraventricular, intracapsular, intravenous, intralymphatical, intraocular or intraperitoneal routes, and wherein the administering method is injection, transfusion, or implantation.

Embodiment II-73. The method of any one of embodiments 11-68 to II-72, wherein the subject is selected from the group consisting of mouse, rat, pig, and non-human primate.

Embodiment II-74. The method of any one of embodiments II-68 to II-72, wherein the subject is a human.

Embodiment II-75. A method of making an rAAV vector, comprising:
a. providing a population of packaging cells; and
b. transfecting the population of cells with:
   i) a vector comprising the polynucleotide of any one of embodiments II-1 to II-57;
   ii) a vector comprising an aap (assembly) gene; and
   iii) a vector comprising the rep and cap genomes.

Embodiment II-76. The method of embodiment II-70, the method further comprising recovering the rAAV vector.

### Set III:

The embodiments of Set III refer to tables provided in the present specification and to sequence listing submitted herewith.

Embodiment III-1. A polynucleotide comprising the following component sequences:
a. a first AAV inverted terminal repeat (ITR) sequence;
b. a second AAV ITR sequence;
c. a first promoter sequence;
d. a sequence encoding a CRISPR protein;
e. a sequence encoding a first guide RNA (gRNA); and,
f. optionally, at least one accessory element sequence,
   wherein the polynucleotide is configured for incorporation into a recombinant adeno-associated virus (AAV).

Embodiment III-2. The polynucleotide of embodiment III-1, wherein the sequences encoding the CRISPR protein and the first gRNA are less than about 3100, less than about 3090, less than about 3080, less than about 3070, less than about 3060, less than about 3050, or less than about 3040 nucleotides in combined length.

Embodiment III-3. The polynucleotide of embodiment III-1 or III-2, wherein the sequences of the first promoter and the at least one accessory element have greater than at least about 1300, at least about 1350, at least about 1360, at least about 1370, at least about 1380, at least about 1390, at least about 1400, at least about 1500, at least about 1600 nucleotides, at least 1650, at least about 1700, at least about 1750, at least about 1800, at least about 1850, or at least about 1900 nucleotides in combined length.

Embodiment III-4. The polynucleotide of embodiment III-1 or III-2, wherein the sequences of the first promoter and the at least one accessory element have greater than 1314 nucleotides in combined length.

Embodiment III-5. The polynucleotide of embodiment III-1 or III-2, wherein the sequences of the first promoter and the at least one accessory element have greater than 1381 nucleotides in combined length.

Embodiment III-6. The polynucleotide of any one of embodiments III-1 to III-5, wherein the first promoter sequence and the sequence encoding the CRISPR protein are operably linked.

Embodiment III-7. The polynucleotide of embodiment III-6, wherein the first promoter is a pol II promoter.

Embodiment III-8. The polynucleotide of embodiment III-6 or III-7, wherein the first promoter is selected from the group consisting of polyubiquitin C (UBC) promoter, cytomegalovirus (CMV) promoter, simian virus 40 (SV40) promoter, chicken beta-Actin promoter and rabbit beta-Globin splice acceptor site fusion (CAG), chicken β-actin promoter with cytomegalovirus enhancer (CB7), PGK promoter, Jens Tornoe (JeT) promoter, GUSB promoter, CBA hybrid (CBh) promoter, elongation factor-1 alpha (EF-1alpha) promoter, beta-actin promoter, Rous sarcoma virus (RSV) promoter, silencing-prone spleen focus forming virus (SFFV) promoter, CMVd1 promoter, truncated human CMV (tCMVd2), minimal CMV promoter, hepB promoter, chicken β-actin promoter, HSV TK promoter, Mini-TK promoter, minimal IL-2 promoter, GRP94 promoter, Super Core Promoter 1, Super Core Promoter 2, Super Core Promoter 3, adenovirus major late (AdML) promoter, MLC promoter, MCK promoter, GRK1 protein promoter, Rho promoter, CAR protein promoter, hSyn Promoter, U1a promoter, Ribosomal Protein Large subunit 30 (Rpl30) promoter, Ribosomal Protein Small subunit 18 (Rps18) promoter, CMV53 promoter, minimal SV40 promoter, CMV53 promoter, SFCp promoter, Mecp2 promoter, pJB42CAT5 promoter, MLP promoter, EFS promoter, MeP426 promoter, MecP2 promoter, MHCK7 promoter, beta-glucuronidase (GUSB) promoter, CK7 promoter, and CK8e promoter.

Embodiment III-9. The polynucleotide of embodiment III-8, wherein the first promoter is a truncated variant of the UBC, CMV, SV40, CAG, CB7, PGK, JeT, GUSB, CB, EF-1alpha, beta-actin, RSV, SFFV, CMVd1, tCMVd2, minimal CMV, chicken β-actin, HSV TK, Mini-TK, minimal IL-2, GRP94, Super Core Promoter 1, Super Core Promoter 2, MLC, MCK, GRK1 protein Rho, CAR protein, hSyn, U1a, Ribosomal Protein Large subunit 30 (Rpl30), Ribosomal Protein Small subunit 18 (Rps18), CMV53, minimal SV40, CMV53, SFCp, pJB42CAT5, MLP, EFS, MeP426, MecP2, MHCK7, CK7, or CK8e promoter.

Embodiment III-10. The polynucleotide of embodiment III-7 or III-8, wherein the first promoter sequence has less than about 400 nucleotides, less than about 350 nucleotides, less than about 300 nucleotides, less than about 200 nucleotides, less than about 150 nucleotides, less than about 100 nucleotides, less than about 80 nucleotides, or less than about 40 nucleotides.

Embodiment III-11. The polynucleotide of embodiment III-7 or III-8, wherein the first promoter sequence has between about 40 to about 585 nucleotides, between about 100 to about 400 nucleotides, or between about 150 to about 300 nucleotides.

Embodiment III-12. The polynucleotide of any one of embodiments III-1 to III-11, wherein the first promoter is selected from the group consisting of SEQ ID NOS: 40370-40400 as set forth in Table 8, or a sequence having at least 85%, at least 90%, at least 95%, at least 95%, at least 96%, at least 97%, at least 98%, or at least 99% identity thereto.

Embodiment III-13. The polynucleotide of any one of embodiments III-1 to III-12, wherein the first promoter is selected from the group consisting of SEQ ID NOS: 41030-41044 as set forth in Table 24, or a sequence having at least 85%, at least 90%, at least 95%, at least 95%, at least 96%, at least 97%, at least 98%, or at least 99% identity thereto.

Embodiment III-14. The polynucleotide of any one of embodiments III-1 to III-13, wherein the at least one accessory element is operably linked to the sequence encoding the CRISPR protein.

Embodiment III-15. The polynucleotide of any one of embodiments III-1 to III-14, further comprising a second promoter.

Embodiment III-16. The polynucleotide of embodiment III-15, wherein the second promoter sequence and the sequence encoding the first gRNA are operably linked.

Embodiment III-17. The polynucleotide of embodiment III-15 or III-16, wherein the second promoter is a pol III promoter.

Embodiment III-18. The polynucleotide of any one of embodiments III-15 to III-17, wherein the second promoter is selected from the group consisting of U6, mini U61, mini U62, mini U63, BiH1 (Bidrectional H1 promoter), BiU6 (Bidirectional U6 promoter), gorilla U6, rhesus U6, human 7sk, and human H1 promoters.

Embodiment III-19. The polynucleotide of embodiment III-18, wherein the second promoter is a truncated variant of the U6, mini U61, mini U62, mini U63, BiH1, BiU6, gorilla U6, rhesus U6, human 7sk, or human H1 promoters.

Embodiment III-20. The polynucleotide of embodiment III-18 or III-19, wherein the second promoter sequence has less than about 250 nucleotides, less than about 220 nucleotides, less than about 200 nucleotides, less than about 160 nucleotides, less than about 140 nucleotides, less than about 130 nucleotides, less than about 120 nucleotides, less than about 100 nucleotides, less than about 80 nucleotides, or less than about 70 nucleotides.

Embodiment III-21. The polynucleotide of embodiment III-18 or III-19, wherein the second promoter sequence has between about 70 to about 245 nucleotides, between about 100 to about 220 nucleotides, or between about 120 to about 160 nucleotides.

Embodiment III-22. The polynucleotide of any one of embodiments III-15 to III-21, wherein the second promoter sequence is selected from the group consisting SEQ ID NOS: 40401-40420 and 41010-41029 as set forth in Table 9, or a sequence having at least 85%, at least 90%, at least 95%, at least 95%, at least 96%, at least 97%, at least 98%, or at least 99% identity thereto.

Embodiment III-23. The polynucleotide of any one of embodiments III-15 to III-22, wherein the second promoter enhances transcription of the first gRNA.

Embodiment III-24. The polynucleotide of any one of embodiments III-15 to III-23, wherein the sequences of the first promoter and the second promoter are greater than at least about 1300, at least about 1350, at least about 1360, at least about 1370, at least about 1380, at least about 1390, at least about 1400, at least about 1500, at least about 1600 nucleotides, at least 1650, at least about 1700, at least about 1750, at least about 1800, at least about 1850, or at least about 1900 nucleotides in combined length.

Embodiment III-25. The polynucleotide of any one of embodiments III-15 to III-24, wherein the sequences of the first promoter, the second promoter and the at least one accessory element are greater than at least about 1300, at least about 1350, at least about 1360, at least about 1370, at least about 1380, at least about 1390, at least about 1400, at least about 1500, at least about 1600 nucleotides, at least 1650, at least about 1700, at least about 1750, at least about 1800, at least about 1850, or at least about 1900 nucleotides in combined length.

Embodiment III-26. The polynucleotide of any one of embodiments 15 to III-25, wherein the sequences of the first promoter, the second promoter, and the at least one accessory element are greater than 1314 nucleotides in combined length.

Embodiment III-27. The polynucleotide of any one of embodiments III-15 to III-26, wherein the sequences of the first promoter, the second promoter, and the at least one accessory element are greater than 1381 nucleotides in combined length.

Embodiment III-28. The polynucleotide of any one of embodiments III-1 to III-27, comprising two or more accessory element sequences.

Embodiment III-29. The polynucleotide of embodiment III-28, wherein the sequences of the first promoter, the second promoter, and the two or more accessory elements are greater than at least about 1300, at least about 1350, at least about 1360, at least about 1370, at least about 1380, at least about 1390, at least about 1400, at least about 1500, at least about 1600, at least 1650, at least about 1700, at least about 1750, at least about 1800, at least about 1850, or greater than at least about 1900 nucleotides in combined length.

Embodiment III-30. The polynucleotide of embodiment III-28, wherein the sequences of the first promoter, the second promoter, and the two or more accessory elements are greater than 1314 nucleotides in combined length.

Embodiment III-31. The polynucleotide of embodiment III-28, wherein the sequences of the first promoter, the second promoter, and the two or more accessory elements are greater than 1381 nucleotides in combined length.

Embodiment III-32. The polynucleotide of any one of embodiment III-15 to III-31, wherein at least 25%, 26%, 27%, 28%, 29%, 30%, 31%, 32%, 33%, 34%, or at least 35% or more of the length of the polynucleotide sequence comprises the sequences of the first and second promoters and the at least one accessory element.

Embodiment III-33. The polynucleotide of any one of embodiments III-1 to III-32, wherein the accessory elements are selected from the group consisting of a poly(A) signal, a gene enhancer element, an intron, a posttranscriptional regulatory element (PTRE), a nuclear localization signal (NLS), a deaminase, a DNA glycosylase inhibitor, a stimulator of CRISPR-mediated homology-directed repair, and an activator of transcription, and a repressor of transcription.

Embodiment III-34. The polynucleotide of any one of embodiments III-1 to III-32, wherein the accessory elements enhance the transcription, transcription termination, expression, binding of a target nucleic acid, editing of a target nucleic acid, or performance of the CRISPR protein as compared to an otherwise identical polynucleotide lacking said accessory elements.

Embodiment III-35. The polynucleotide of embodiment III-34, wherein the enhanced performance is an increase in editing of a target nucleic acid by the expressed CRISPR protein and the first gRNA in an in vitro assay of at least about 10%, at least about 20%, at least about 30%, at least about 40%, at least about 50%, at least about 60%, at least about 70%, at least about 80%, at least about 90%, at least about 100%, at least about 150%, at least about 200%, or at least about 300%.

Embodiment III-36. The polynucleotide of any one of embodiments III-1 to III-35, wherein the encoded CRISPR protein is a Class 2 CRISPR protein.

Embodiment III-37. The polynucleotide of embodiment III-36, wherein the encoded CRISPR protein is a Class 2, Type V CRISPR protein.

Embodiment III-38. The polynucleotide of embodiment III-37, wherein the encoded Class 2, Type V CRISPR protein comprises:
a. a NTSB domain comprising a sequence of QPASKKIDQNKLKPEMDEKGNLTTAGFACSQCGQPLFVYKLEQVSEKGKAYTN YFGRCNVAEHEKLILLAQLKPEKDSDEAVTYSLGKFGQ (SEQ ID NO: 41818), or a sequence having at least 80% at least 90%, at least 95%, at least 96%, at least 97%, at least 98% or at least 99% identity thereto;
b. a helical I-II domain comprising a sequence of RALDFYSIHVTKESTHPVKPLAQIAGNRYASGPVGKALSDACMGTIASFLSKYQD IIIEHQKVVKGNQKRLESLRELAGKENLEYPSVTLPPQPHTKEGVDAYNEVIARV RMWVNLNLWQKLKLSRDDAKPLLRLKGFPSF (SEQ ID NO: 41819), or a sequence having at least 80%, at least 90%, at least 95%, at least 96%, at least 97%, at least 98% or at least 99% identity thereto;
c. a helical II domain comprising a sequence of PLVERQANEVDWWDMVCNVKKLINEKKEDGKVFWQNLAGYKRQEALRPYLSS EEDRKKGKKFARYQLGDLLLHLEKKHGEDWGKVYDEAWERIDKKVEGLSKHI KLEEERRSEDAQSKAALTDWLRAKASFVIEGLKEADKDEFCRCELKLQKWYGD LRGKPFAIEAE (SEQ ID NO: 41820), or a sequence having at least 80%, at least 90%, at least 95%, at least 96%, at least 97%, at least 98% or at least 99% identity thereto; and
d. a RuvC-I domain comprising a sequence of SSNIKPMNLIGVDRGENIPAVIALTDPEGCPLSRFKDSLGNPTHILRIGESYKEKQR TIQAKKEVEQRRAGGYSRKYASKAKNLADDMVRNTARDLLYYAVTQDAMLIF ENLSRGFGRQGKRTFMAERQYTRMEDWLTAKLAYEGLPSKTYLSKTLAQYTSK TC (SEQ ID NO: 41821), or a sequence having at least 80%, at least 90%, at least 95%, at least 96%, at least 97%, at least 98% or at least 99% identity thereto.

Embodiment III-39. The polynucleotide of embodiment III-38, wherein the encoded Class 2, Type V CRISPR protein comprises an OBD-I domain comprising a sequence of QEIKRINKIRRRLVKDSNTKKAGKTGPMKTLLVRVMTPDLRERLENLRKKPENIPQ (SEQ ID NO: 41822), or a sequence having at least 80%, at least 90%, at least 95%, at least 96%, at least 97%, at least 98%, or at least 99% identity thereto.

Embodiment III-40. The polynucleotide of embodiment III-38 or III-39, wherein the encoded Class 2, Type V CRISPR protein comprises an OBD-II domain comprising a sequence of NSILDISGFSKQYNCAFIWQKDGVKKLNLYLIINYFKGGKLRFKKIKPEAFEANRFYTVIN KKSGEIVPMEVNFNFDDPNLIILPLAFGKRQGREFIWNDLLSLETGSLKLANGRVIEKTL YNRRTRQDEPALFVALTFERREVLD (SEQ ID NO: 41823), or a sequence having at least 80%, at least 90%, at least 95%, at least 96%, at least 97%, at least 98%, or at least 99% identity thereto.

Embodiment III-41. The polynucleotide of any one of embodiments III-38 to III-40, wherein the encoded Class 2, Type V CRISPR protein comprises a helical I-I domain comprising a sequence of PISNTSRANLNKLLTDYTEMKKAILHVYWEEFQKDPVGLMSRVA (SEQ ID NO: 41824), or a sequence having at least 80%, at least 90%, at least 95%, at least 96%, at least 97%, at least 98%, or at least 99% identity thereto.

Embodiment III-42. The polynucleotide of any one of embodiments III-38 to III-41, wherein the encoded Class 2, Type V CRISPR protein comprises a TSL domain comprising a sequence of SNCGFTITSADYDRVLEKLKKTATGWMTTINGKELKVEGQITYYNRYKRQNVVKDLSV ELDRLSEESVNNDISSWTKGRSGEALSLLKKRFSHRPVQEKFVCLNCGFETH (SEQ ID NO: 41825), or a sequence having at least 80%, at least 90%, at least 95%, at least 96%, at least 97%, at least 98%, or at least 99% identity thereto.

Embodiment III-43. The polynucleotide of any one of embodiments III-38 to III-42, wherein the encoded Class 2, Type V CRISPR protein comprises a RuvC-II domain comprising a sequence of ADEQAALNIARSWLFLRSQEYKKYQTNKTTGNTDKRAFVETWQSFYRKKLKEVWKPA V (SEQ ID NO: 41826), or a sequence having at least 80%, at least 90%, at least 95%, at least 96%, at least 97%, at least 98%, or at least 99% identity thereto.

Embodiment III-44. The polynucleotide of any one of embodiments III-38 to III-43, wherein the encoded Class 2, Type V CRISPR protein comprises the sequence of SEQ ID NO: 145, or a sequence having at least 80%, at least 90%, at least 95%, at least 96%, at least 97%, at least 98%, or at least 99% identity thereto.

Embodiment III-45. The polynucleotide of any one of embodiments III-38 to III-44, wherein the encoded Class 2, Type V CRISPR protein comprises at least one modification in one or more domains.

Embodiment III-46. The polynucleotide of embodiment III-45, wherein the at least one modification comprises:
a. at least one amino acid substitution in a domain;
b. at least one amino acid deletion in a domain;
c. at least one amino acid insertion in a domain; or
d. any combination of (a)-(c).

Embodiment III-47. The polynucleotide of embodiment III-45 or III-46, comprising a modification at one or more amino acid positions in the NTSB domain relative to SEQ ID NO: 41818 selected from the group consisting of P2, S4, Q9, E15, G20, G33, L41, Y51, F55, L68, A70, E75, K88, and G90.

Embodiment III-48. The polynucleotide of embodiment III-47, wherein the one or more modifications at one or more amino acid positions in the NTSB domain are selected from the group consisting of an insertion of G at position 2, an insertion of I at position 4, an insertion of L at position 4, Q9P, E15S, G20D, a deletion of S at position 30, G33T, L41A, Y51T, F55V, L68D, L68E, L68K, A70Y, A70S, E75A, E75D, E75P, K88Q, and G90Q relative to SEQ ID NO: 41818.

Embodiment III-49. The polynucleotide of any one of embodiments III-45 to III-48, comprising a modification at one or more amino acid positions in the helical I-II domain relative to SEQ ID NO: 41819 selected from the group consisting of 124, A25, Y29 G32, G44, S48, S51, Q54, 156, V63, S73, L74, K97, V100, M112, L116, G137, F138, and S140.

Embodiment III-50. The polynucleotide of embodiment III-49, wherein the one or more modifications at one or more amino acid positions in the helical I-II domain are selected from the group consisting of an insertion of T at position 24, an insertion of C at position 25, Y29F, G32Y, G32N, G32H, G32S, G32T, G32A, G32V, a deletion of G at position 32, G32S, G32T, G44L, G44H, S48H, S48T, S51T, Q54H, I56T, V63T, S73H, L74Y, K97G, K97S, K97D, K97E, V100L, M112T, M112W, M112R, M112K, L116K, G137R, G137K, G137N, an insertion of Q at position 138, and S140Q relative to SEQ ID NO: 41819.

Embodiment III-51. The polynucleotide of any one of embodiments III-45 to III-50, comprising a modification at one or more amino acid positions in the helical II domain relative to SEQ ID NO: 41820 selected from the group consisting of L2, V3, E4, R5, Q6, A7, E9, V10, D11, W12, W13, D14, M15, V16, C17, N18, V19, K20, L22, I23, E25, K26, K31, Q35, L37, A38, K41, R 42, Q43, E44, L46, K57, Y65, G68, L70, L71, L72, E75, G79, D81, W82, K84, V85, Y86, D87, 193, K95, K96, E98, L100, K102, 1104, K105, E109, R 110, D 114, K 118, A120, L121, W124, L125, R126, A127, A129, 1133, E134, G135, L136, E138, D140, K141, D142, E143, F144, C145, C147, E148, L149, K150, L151, Q152, K153, L158, E166, and A167.

Embodiment III-52. The polynucleotide of embodiment III-51, wherein the one or more modifications at one or more amino acid positions in the helical II domain are selected from the group consisting of an insertion of A at position 2, an insertion of H at position 2, a deletion of L at position 2 and a deletion of V at position 3, V3E, V3Q, V3F, a deletion of V at position 3, an insertion of D at position 3, V3P, E4P, a deletion of E at position 4, E4D, E4L, E4R, R5N, Q6V, an insertion of Q at position 6, an insertion of G at position 7, an insertion of H at position 9, an insertion of A at position 9, VD10, an insertion of T1 at position 0, a deletion of V at position 10, an insertion of F at position 10, an insertion of D at position 11, a deletion of D at position 11, D11S, a deletion of W at position 12, W12T, W12H, an insertion of P at position 12, an insertion of Q at position 13, an insertion of G at position 12, an insertion of R at position 13, W13P, W13D, an insertion of D at position 13, W13L, an insertion of P at position 14, an insertion of D at position 14, a deletion of D at position 14 and a deletion of M at position 15, a deletion of M at position 15, an insertion of T at position 16, an insertion of P at position 17, N18I, V19N, V19H, K20D, L22D, 123S, E25C, E25P, an insertion of G at position 25, K26T, K27E, K31L, K31Y, Q35D, Q35P, an insertion of S at position 37, a deletion of L at position 37 and a deletion of A at position 38, K41L, an insertion of R at position 42, a deletion of Q at position 43 and a deletion of E at position 44, L46N, K57Q, Y65T, G68M, L70V, L71C, L72D, L72N, L72W, L72Y, E75F, E75L, E75Y, G79P, an insertion of E at position 79, an insertion of T at position 81, an insertion of R at position 81, an insertion of W at position 81, an insertion of Y at position 81, an insertion of W at position 82, an insertion of Y at position 82, W82G, W82R, K84D, K84H, K84P, K84T, V85L, V85A, an insertion of L at position 85, Y86C, D87G, D87M, D87P, I93C, K95T, K96R, E98G, L100A, K102H, I104T, I104S, I104Q, K105D, an insertion of K at position 109, E109L, R110D, a deletion of R at position 110, D114E, an insertion of D at position 114, K118P, A120R, L121T, W124L, L125C, R126D, A127E, A127L, A129T, A129K, I133E, an insertion of C at position 133, an insertion of S at position 134, an insertion of G at position 134, an insertion of R at position 135, G135P, L136K, L136D, L136S, L136H, a deletion of E at position 138, D140R, an insertion of D at position 140, an insertion of P at position 141, an insertion of D at position 142, a deletion of E at position 143+a deletion of F at position 144, an insertion of Q at position 143, F144K, a deletion of F at position 144, a deletion of F at position 144 and a deletion of C at position 145, C145R, an insertion of G at position 145, C145K, C147D, an insertion of V at position 148, E148D, an insertion of H at position 149, L149R, K150R, L151H, Q152C, K153P, L158S, E166L, and an insertion of F at position 167 relative to SEQ ID NO: 41820.

Embodiment III-53. The polynucleotide of any one of embodiments III-45 to III-52, comprising a modification at one or more amino acid positions in the RuvC-I domain relative to SEQ ID NO: 41821 selected from the group consisting of 14, K5, P6, M7, N8, L9, V12, G49, K63, K80, N83, R90, M125, and L146.

Embodiment III-54. The polynucleotide of embodiment III-53, wherein the one or more modifications at one or more amino acid positions in the RuvC-I domain are selected from the group consisting of an insertion of I at position 4, an insertion of S at position 5, an insertion of T at position 6, an insertion of N at position 6, an insertion of R at position 7, an insertion of K at position 7, an insertion of H at position 8, an insertion of S at position 8, V12L, G49W, G49R, S51R, S51K, K62S, K62T, K62E, V65A, K80E, N83G, R90H, R90G, M125S, M125A, L137Y, an insertion of P at position 137, a deletion of L at position 141, L141R, L141D, an insertion of Q at position 142, an insertion of R at position 143, an insertion of N at position 143, E144N, an insertion of P at position 146, L146F, P147A, K149Q, T150V, an insertion of R at position 152, an insertion of H153, T155Q, an insertion of H at position 155, an insertion of R at position 155, an insertion of L at position 156, a deletion of L at position 156, an insertion of W at position 156, an insertion of A at position 157, an insertion of F at position 157, A157S, Q158K, a deletion of Y at position 159, T160Y, T160F, an insertion of I at position 161, S161P, T163P, an insertion of N at position 163, C164K, and C164M relative to SEQ ID NO: 41821.

Embodiment III-55. The polynucleotide of any one of embodiments III-45 to III-54, comprising a modification at one or more amino acid positions in the OBD-I domain relative to SEQ ID NO: 41822 selected from the group consisting of I3, K4, R5, 16, N7, K8, K15, D16, N18, P27, M28, V33, R34, M36, R41, L47, R48, E52, P55, and Q56.

Embodiment III-56. The polynucleotide of embodiment III-55, wherein the one or more modifications at one or more amino acid positions in the OBD-I domain are selected from the group consisting of an insertion of G at position 3, I3G, I3E, an insertion of G at position 4, K4G, K4P, K4S, K4W, K4W, R5P, an insertion of P at position 5, an insertion of G at position 5, R5S, an insertion of S at position 5, R5A, R5P, R5G, R5L, I6A, I6L, an insertion of G at position 6, N7Q, N7L, N7S, K8G, K15F, D16W, an insertion of F at position 16, an insertion of F18, an insertion of P at position 27, M28P, M28H, V33T, R34P, M36Y, R41P, L47P, an insertion of P at position 48, E52P, an insertion of P at position 55, a deletion of P at position 55 and a deletion of Q at position 56, Q56S, Q56P, an insertion of D at position 56, an insertion of T at position 56, and Q56P relative to SEQ ID NO: 41822.

Embodiment III-57. The polynucleotide of any one of embodiments III-45 to III-56, comprising a modification at one or more amino acid positions in the OBD-II domain relative to SEQ ID NO: 41823 selected from the group consisting of S2, I3, L4, K11, V24, K37, R42, A53, T58, K63, M70, 182, Q92, G93, K110, L121, R124, R141, E143, V144, and L145.

Embodiment III-58. The polynucleotide of embodiment III-57, wherein the one or more modifications at one or more amino acid positions in the OBD-II domain are selected from the group consisting of a deletion of S at position 2, I3R, I3K, a deletion of I at position 3 and a deletion of L4, a deletion of L at position 4, K11T, an insertion of P at position 24, K37G, R42E, an insertion of S at position 53, an insertion of R at position 58, a deletion of K at position 63, M70T, I82T, Q92I, Q92F, Q92V, Q92A, an insertion of A at position 93, K110Q, R115Q, L121T, an insertion of A at position 124, an insertion of R at position 141, an insertion of D at position 143, an insertion of A at position 143, an insertion of W at position 144, and an insertion of A at position 145 relative to SEQ ID NO: 41823. 105511 Embodiment III-59. The polynucleotide of any one of embodiments III-45 to III-58, comprising a modification at one or more amino acid positions in the TSL domain relative to SEQ ID NO: 41825 selected from the group consisting of S1, N2, C3, G4, F5, 17, K18, V58, S67, T76, G78, S80, G81, E82, S85, V96, and E98.

Embodiment III-60. The polynucleotide of embodiment III-59, wherein the one or more modifications at one or more amino acid positions in the OBD-II domain are selected from the group consisting of an insertion of M at position 1, a deletion of N at position 2, an insertion of V at position 2, C3S, an insertion of G at position 4, an insertion of W at position 4, F5P, an insertion of W at position 7, K18G, V58D, an insertion of A at position 67, T76E, T76D, T76N, G78D, a deletion of S at position 80, a deletion of G at position 81, an insertion of E at position 82, an insertion of N at position 82, S85I, V96C, V96T, and E98D relative to SEQ ID NO: 41825.

Embodiment III-61. The polynucleotide of any one of embodiments III-45 to III-60, wherein the expressed Class 2, Type V CRISPR protein exhibits an improved characteristic relative to SEQ ID NO: 2 or SEQ ID NO: 145, wherein the improved characteristic comprises increased binding affinity to a gRNA, increased binding affinity to the target nucleic acid, improved ability to utilize a greater spectrum of PAM sequences in the editing of the target nucleic acid, improved unwinding of the target nucleic acid, increased editing activity, improved editing efficiency, improved editing specificity for cleavage of the target nucleic acid, decreased off-target editing or cleavage of the target nucleic acid, increased percentage of a eukaryotic genome that can be edited, increased activity of the nuclease, increased target strand loading for double strand cleavage, decreased target strand loading for single strand nicking, increased binding of the non-target strand of DNA, improved protein stability, increased protein:gRNA (RNP) complex stability, and improved fusion characteristics.

Embodiment III-62. The polynucleotide of embodiment III-61, wherein the improved characteristic comprises increased cleavage activity at a target nucleic sequence comprising an TTC, ATC, GTC, or CTC PAM sequence.

Embodiment III-63. The polynucleotide of embodiment III-62, wherein the improved characteristic comprises increased cleavage activity at a target nucleic acid sequence comprising an ATC or CTC PAM sequence relative to cleavage activity of the sequence of SEQ ID NO: 145. 105561 Embodiment III-64. The polynucleotide of embodiment III-63, wherein the improved cleavage activity is an enrichment score (log₂) of at least about 1.5, at least about 2.0, at least about 2.5, at least about 3, at least about 3.5, at least about 4, at least about 4.5, at least about 5, at least about 6, at least about 7, at least about 8 or more greater compared to score of the sequence of SEQ ID NO: 145 in an in vitro assay.

Embodiment III-65. The polynucleotide of embodiment III-63, wherein the improved characteristic comprises increased cleavage activity at a target nucleic acid sequence comprising an CTC PAM sequence relative to the sequence of SEQ ID NO: 145.

Embodiment III-66. The polynucleotide of embodiment III-65, wherein the improved cleavage activity is an enrichment score (log₂) of at least about 2, at least about 2.5, at least about 3, at least about 3.5, at least about 4, at least about 4.5, at least about 5, or at least about 6 or more greater compared to the score of the sequence of SEQ ID NO: 145 in an in vitro assay.

Embodiment III-67. The polynucleotide of embodiment III-62, wherein the improved characteristic comprises increased cleavage activity at a target nucleic acid sequence comprising an TTC PAM sequence relative to the sequence of SEQ ID NO: 145.

Embodiment III-68. The polynucleotide of embodiment III-67, wherein the improved cleavage activity is an enrichment score of at least about 1.5, at least about 2.0, at least about 2.5, at least about 3, at least about 3.5, at least about 4, at least about 4.5, at least about 5, or at least about 6 log 2 or more greater compared to the sequence of SEQ ID NO: 145 in an in vitro assay.

Embodiment III-69. The polynucleotide of embodiment III-61, wherein the improved characteristic comprises increased specificity for cleavage of the target nucleic acid sequence relative to the sequence of SEQ ID NO: 145.

Embodiment III-70. The polynucleotide of embodiment III-69, wherein the increased specificity is an enrichment score of at least about 2.0, at least about 2.5, at least about 3, at least about 3.5, at least about 4, at least about 4.5, at least about 5, or at least about 6 log 2 or more greater compared to the sequence of SEQ ID NO: 145 in an in vitro assay.

Embodiment III-71. The polynucleotide of embodiment III-61, wherein the improved characteristic comprises decreased off-target cleavage of the target nucleic acid sequence.

Embodiment III-72. The polynucleotide of embodiment III-37, wherein the encoded Class 2, Type V CRISPR protein is selected from the group consisting of Cas12f, Cas12j (CasPhi), and CasX.

Embodiment III-73. The polynucleotide of embodiment III-72, wherein the encoded CasX comprises a sequence selected from the group consisting of SEQ ID NOS: 1-3, 49-160, and 40208-40369, or a sequence having at least 85%, at least 90%, at least 95%, at least 95%, at least 96%, at least 97%, at least 98%, or at least 99% identity thereto.

Embodiment III-74. The polynucleotide of embodiment III-72, wherein the encoded CasX comprises a sequence selected from the group consisting of the sequences of SEQ ID NOS: 1-3, 49-160, 40208-40369 and 40828-40912.

Embodiment III-75. The polynucleotide of embodiment III-72, wherein the CasX sequence of the polynucleotide comprises a sequence selected from the group consisting of SEQ ID NOS: 40577-40588, as set forth in Table 21, or a sequence having at least 85%, at least 90%, at least 95%, at least 95%, at least 96%, at least 97%, at least 98%, or at least 99% identity thereto.

Embodiment III-76. The polynucleotide of embodiment III-72, wherein the CasX sequence of the polynucleotide comprises a sequence selected from the group consisting of SEQ ID NOS: 40577-40588, as set forth in Table 21.

Embodiment III-77. The polynucleotide of any one of embodiments III-1 to III-76, wherein the polynucleotide encodes one or more NLS linked to the sequence encoding the CRISPR protein.

Embodiment III-78. The polynucleotide of embodiment III-77, wherein the sequences encoding the one or more NLS are positioned at or near the 5′ end of the sequence encoding the CRISPR protein.

Embodiment III-79. The polynucleotide of embodiment III-78 or III-79, wherein the sequences encoding the one or more NLS are positioned at or near at the 3′ end of the sequence encoding the CRISPR protein.

Embodiment III-80. The polynucleotide of embodiment III-78 or III-79, wherein the polynucleotide encodes at least two NLS, wherein the sequences encoding the at least two NLS are positioned at or near the 5′ and 3′ ends of the sequence encoding the CRISPR protein.

Embodiment III-81. The polynucleotide of any one of embodiments III-77 to III-80, wherein the one or more encoded NLS are selected from the group of sequences consisting of PKKKRKV (SEQ ID NO: 196), KRPAATKKAGQAKKKK (SEQ ID NO: 197), PAAKRVKLD (SEQ ID NO: 248), RQRRNELKRSP (SEQ ID NO: 161), NQSSNFGPMKGGNFGGRSSGPYGGGGQYFAKPRNQGGY (SEQ ID NO: 162), RMRIZFKNKGKDTAELRRRRVEVSVELRKAKKDEQILKRRNV (SEQ ID NO: 163), VSRKRPRP (SEQ ID NO: 164), PPKKARED (SEQ ID NO: 165), PQPKKKPL (SEQ ID NO: 166), SALIKKKKKMAP (SEQ ID NO: 167), DRLRR (SEQ ID NO: 168), PKQKKRK (SEQ ID NO: 169), RKLKKKIKKL (SEQ ID NO: 170), REKKKFLKRR (SEQ ID NO: 171), KRKGDEVDGVDEVAKKKSKK (SEQ ID NO: 172), RKCLQAGMNLEARKTKK (SEQ ID NO: 173), PRPRKIPR (SEQ ID NO: 174), PPRKKRTVV (SEQ ID NO: 175), NLSKKKKRKREK (SEQ ID NO: 176), RRPSRPFRKP (SEQ ID NO: 177), KRPRSPSS (SEQ ID NO: 178), KRGINDRNFWRGENERKTR (SEQ ID NO: 179), PRPPKMARYDN (SEQ ID NO: 180), KRSFSKAF (SEQ ID NO: 181), KLKIKRPVK (SEQ ID NO: 182), PKKKRKVPPPPAAKRVKLD (SEQ ID NO: 183), PKTRRRPRRSQRKRPPT (SEQ ID NO: 184), SRRRKANPTKLSENAKKLAKEVEN (SEQ ID NO: 41827), KTRRRPRRSQRKRPPT (SEQ ID NO: 186), RRKKRRPRRKKRR (SEQ ID NO: 187), PKKKSRKPKKKSRK (SEQ ID NO: 188), HKKKHPDASVNFSEFSK (SEQ ID NO: 189), QRPGPYDRPQRPGPYDRP (SEQ ID NO: 190), LSPSLSPLLSPSLSPL (SEQ ID NO: 191), RGKGGKGLGKGGAKRHRK (SEQ ID NO: 192), PKRGRGRPKRGRGR (SEQ ID NO: 193), PKKKRKVPPPPKKKRKV (SEQ ID NO: 195), PAKRARRGYKC (SEQ ID NO: 40188), KLGPRKATGRW (SEQ ID NO: 40189), PRRKREE (SEQ ID NO: 40190), PYRGRKE (SEQ ID NO: 40191), PLRKRPRR (SEQ ID NO: 40192), PLRKRPRRGSPLRKRPRR (SEQ ID NO: 40193), PAAKRVKLDGGKRTADGSEFESPKKKRKV (SEQ ID NO: 40194), PAAKRVKLDGGKRTADGSEFESPKKKRKVGIHGVPAA (SEQ ID NO: 40195), PAAKRVKLDGGKRTADGSEFESPKKKRKVAEAAAKEAAAKEAAAKA (SEQ ID NO: 40196), PAAKRVKLDGGKRTADGSEFESPKKKRKVPG (SEQ ID NO: 40710), KRKGSPERGERKRHW (SEQ ID NO: 40198), KRTADSQHSTPPKTKRKVEFEPKKKRKV (SEQ ID NO: 41828), and PKKKRKVGGSKRTADSQHSTPPKTKRKVEFEPKKKRKV (SEQ ID NO: 40200) wherein the one or more NLS are linked to the CRISPR variant or to adjacent NLS with a linker peptide wherein the linker peptide is selected from the group consisting of RS, (G)n (SEQ ID NO: 40201), (GS)n (SEQ ID NO: 40202), (GSGGS)n (SEQ ID NO: 208), (GGSGGS)n (SEQ ID NO: 209), (GGGS)n (SEQ ID NO: 210), GGSG (SEQ ID NO: 211), GGSGG (SEQ ID NO: 212), GSGSG (SEQ ID NO: 213), GSGGG (SEQ ID NO: 214), GGGSG (SEQ ID NO: 215), GSSSG (SEQ ID NO: 216), GPGP (SEQ ID NO: 217), GGP, PPP, PPAPPA (SEQ ID NO: 218), PPPG (SEQ ID NO: 40207), PPPGPPP (SEQ ID NO: 219), PPP(GGGS)n (SEQ ID NO: 40203), (GGGS)nPPP (SEQ ID NO: 40204), AEAAAKEAAAKEAAAKA (SEQ ID NO: 40205), and TPPKTKRKVEFE (SEQ ID NO: 40206), wherein n is 1 to 5.

Embodiment III-82. The polynucleotide of any one of embodiments III-77 to III-80, wherein the one or more encoded NLS are selected from the group consisting of SEQ ID NOS: 40443-40501 as set forth in Table 15 and Table 16, or a sequence having at least 85%, at least 90%, at least 95%, at least 95%, at least 96%, at least 97%, at least 98% identity thereto.

Embodiment III-83. The polynucleotide of any one of embodiments III-77 to III-80, wherein the one or more encoded NLS are selected from the group of sequences consisting of SEQ ID NOS: 40443-40501 as set forth in Table 15 and Table 16.

Embodiment III-84. The polynucleotide of any one of embodiments III-1 to III-83, wherein the encoded first gRNA comprises a sequence selected from the group consisting of SEQ ID NOS: 2101-2285, 39981-40026, 40913-40958, and 41817 as set forth in Table 2, or a sequence having at least 85%, at least 90%, at least 95%, at least 95%, at least 96%, at least 97%, at least 98% identity thereto.

Embodiment III-85. The polynucleotide of any one of embodiments III-1 to III-84, wherein the encoded first gRNA comprises a sequence selected from the group consisting of SEQ ID NOS: 2101-2285, 39981-40026, 40913-40958, and 41817 as set forth in Table 2.

Embodiment III-86. The polynucleotide of embodiment III-85, wherein the encoded first gRNA comprises a targeting sequence complementary to a target nucleic acid sequence, wherein the targeting sequence has at least 15 to 30 nucleotides.

Embodiment III-87. The polynucleotide of embodiment III-86, wherein the targeting sequence has 18, 19, or 20 nucleotides.

Embodiment III-88. The polynucleotide of any one of embodiments III-1 to III-87, comprising a sequence encoding a second gRNA and a third promoter operably linked to the second gRNA.

Embodiment III-89. The polynucleotide of embodiment III-88, wherein the third promoter is a pol III promoter.

Embodiment III-90. The polynucleotide of embodiment III-88 or III-89, wherein the third promoter is selected from the group consisting of U6, mini U61, mini U62, mini U63, BiH1 (Bidrectional H1 promoter), BiU6 (Bidirectional U6 promoter), gorilla U6, rhesus U6, human 7sk, and human H1 promoters.

Embodiment III-91. The polynucleotide of embodiment III-90, wherein the third promoter is a truncated variant of the U6, mini U61, mini U62, mini U63, BiH1, BiU6, gorilla U6, rhesus U6, human 7sk, or human H1 promoters.

Embodiment III-92. The polynucleotide of any one of embodiments III-88 to III-91, wherein the third promoter has less than about 250 nucleotides, less than about 220 nucleotides, less than about 200 nucleotides, less than about 160 nucleotides, less than about 140 nucleotides, less than about 130 nucleotides, less than about 120 nucleotides, less than about 100 nucleotides, less than about 80 nucleotides, or less than about 70 nucleotides.

Embodiment III-93. The polynucleotide of any one of embodiments III-88 to III-91, wherein the third promoter has between about 70 to about 245 nucleotides, between about 100 to about 220 nucleotides, or between about 120 to about 160 nucleotides.

Embodiment III-94. The polynucleotide of any one of embodiments III-88 to III-93, wherein the third promoter is selected from the group consisting SEQ ID NOS: 40401-40420 and 41010-41029 as set forth in Table 9, or a sequence having at least 85%, at least 90%, at least 95%, at least 95%, at least 96%, at least 97%, at least 98%, or at least 99% identity thereto.

Embodiment III-95. The polynucleotide of any one of embodiments III-88 to III-94, wherein the third promoter enhances transcription of the second gRNA.

Embodiment III-96. The polynucleotide of any one of embodiments III-88 to III-95, wherein the encoded second gRNA comprises a sequence selected from the group consisting of SEQ ID NOS: 2101-2285, and 39981-40026, 40913-40958, and 41817 as set forth in Table 2, or a sequence having at least 85%, at least 90%, at least 95%, at least 95%, at least 96%, at least 97%, at least 98% identity thereto.

Embodiment III-97. The polynucleotide of any one of embodiments III-88 to III-95, wherein the encoded second gRNA comprises a sequence selected from the group consisting of SEQ ID NOS: 2101-2285, 39981-40026, 40913-40958, and 41817 as set forth in Table 2.

Embodiment III-98. The polynucleotide of any one of embodiments III-89 to III-97, wherein the encoded second gRNA comprises a targeting sequence complementary to a target nucleic acid sequence different than the target nucleic acid of embodiment III-86 or embodiment III-87, wherein the targeting sequence has at least 15 to 30 nucleotides.

Embodiment III-99. The polynucleotide of embodiment III-98, wherein the targeting sequence has 18, 19, or 20 nucleotides.

Embodiment III-100. The polynucleotide of any one of embodiments III-86 to III-99, wherein the targeting sequence is selected from the group consisting of SEQ ID NOS: 41056-41776 as set forth in Table 27, or a sequence having at least 80%, at least 90%, or at least 95% sequence identity thereto.

Embodiment III-101. The polynucleotide of any one of embodiments III-86 to III-99, wherein the targeting sequence is selected from the group consisting of SEQ ID NOS: 41056-41776 as set forth in Table 27.

Embodiment III-102. The polynucleotide of any one of embodiments III-86 to III-101, wherein the encoded first and second gRNA comprise a scaffold sequence having one or more modifications relative to SEQ ID NO: 2238, wherein the one or more modifications result in an improved characteristic in the expressed first and second gRNA.

Embodiment III-103. The polynucleotide of embodiment III-102, wherein the one or more modifications comprise one or more nucleotide substitutions, insertions, and/or deletions as set forth in Table 28.

Embodiment III-104. The polynucleotide of embodiment III-102 or III-103, wherein the improved characteristic is one or more functional properties selected from the group consisting of increased editing activity, increased pseudoknot stem stability, increased triplex region stability, increased scaffold stem stability, extended stem stability, reduced off-target folding intermediates, and increased binding affinity to a Class 2, Type V CRISPR protein, optionally in an in vitro assay.

Embodiment III-105. The polynucleotide of any one of embodiments III-102 to III-104, wherein the expressed gRNA scaffold exhibits an improved enrichment score (log₂) of at least about 2.0, at least about 2.5, at least about 3, or at least about 3.5 greater compared to the score of the gRNA scaffold of SEQ ID NO: 2238 in an in vitro assay.

Embodiment III-106. The polynucleotide of embodiments III-84 to III-101, wherein the encoded first and second gRNA comprise a scaffold sequence having one or more modifications relative to SEQ ID NO: 2239, wherein the one or more modifications result in an improved characteristic in the expressed first and second gRNA.

Embodiment III-107. The polynucleotide of embodiment III-106, wherein the one or more modifications comprise one or more nucleotide substitutions, insertions, and/or deletions as set forth in Table 29. 106001 Embodiment III-108. The polynucleotide of embodiment III-106 or III-107, wherein the improved characteristic is one or more functional properties selected from the group consisting of increased editing activity, increased pseudoknot stem stability, increased triplex region stability, increased scaffold stem stability, extended stem stability, reduced off-target folding intermediates, and increased binding affinity to a Class 2, Type V CRISPR protein, optionally in an in vitro assay.

Embodiment III-109. The polynucleotide of any one of embodiments III-106 to III-108, wherein the expressed gRNA scaffold exhibits an improved enrichment score (log₂) of at least about 1.2, at least about 1.5, at least about 2.0, at least about 2.5, at least about 3, or at least about 3.5 greater compared to the score of the gRNA scaffold of SEQ ID NO: 2239 in an in vitro assay.

Embodiment III-110. The polynucleotide of any one of embodiments III-106 to III-109, comprising one or more modifications at positions relative to the sequence of SEQ ID NO: 2239 selected from the group consisting of C9, U11, C17, U24, A29, U54, G64, A88, and A95.

Embodiment III-111. The polynucleotide of embodiment III-110, comprising one or more modifications relative to the sequence of SEQ ID NO: 2239 selected from the group consisting of C9U, U11C, C17G, U24C, A29C, an insertion of G at position 54, an insertion of C at position 64, A88G, and A95G.

Embodiment III-112. The polynucleotide of embodiment III-111, comprising modifications relative to the sequence of SEQ ID NO: 2239 consisting of C9U, U11C, C17G, U24C, A29C, an insertion of G at position 54, an insertion of C at position 64, A88G, and A95G.

Embodiment III-113. The polynucleotide of any one of embodiments III-106 to III-112, wherein the improved characteristic is selected from the group consisting of pseudoknot stem stability, triplex region stability, scaffold bubble stability, extended stem stability, and binding affinity to a Class 2, Type V CRISPR protein.

Embodiment III-114. The polynucleotide of embodiment III-112, wherein the insertion of C at position 64 and the A88G substitution relative to the sequence of SEQ ID NO: 2239 resolves an asymmetrical bulge element of the extended stem, enhancing the stability of the extended stem of the gRNA scaffold.

Embodiment III-115. The polynucleotide of embodiment III-112, wherein the substitutions of U11C, U24C, and A95G increase the stability of the triplex region of the gRNA scaffold.

Embodiment III-116. The polynucleotide of embodiment III-112, wherein the substitution of A29C increases the stability of the pseudoknot stem.

Embodiment III-117. The polynucleotide of any one of embodiments III-1 to III-116, wherein the accessory element is a post-transcriptional regulatory element (PTRE) selected from the group consisting of cytomegalovirus immediate/early intronA, hepatitis B virus PRE (HPRE), Woodchuck Hepatitis virus PRE (WPRE), and 5′ untranslated region (UTR) of human heat shock protein 70 mRNA (Hsp70).

Embodiment III-118. The polynucleotide of embodiment III-117, wherein the accessory element is a PTRE selected from the group consisting SEQ ID NOS: 40431-40442 as set forth in Table 12, or a sequence having at least 85%, at least 90%, at least 95%, at least 95%, at least 96%, at least 97%, at least 98% identity thereto.

Embodiment III-119. The polynucleotide of any one of embodiments III-1 to III-118, wherein the 5′ and 3′ ITRs are derived from serotype AAV1, AAV2, AAV3, AAV4, AAV5, AAV6, AAV7, AAV8, AAV9, AAV10, AAV11, AAV12, AAV 44.9, AAV-Rh74, or AAVRh10.

Embodiment III-120. The polynucleotide of embodiment III-119, wherein the 5′ and 3′ ITRs are derived from serotype AAV2.

Embodiment III-121. The polynucleotide of any one of embodiments III-1 to III-120, comprising one or more sequences selected from the group consisting of the sequences of Tables 8-10, 12, 13, 17-22 and 24-27, or a sequence having at least 85%, at least 90%, at least 95%, at least 95%, at least 96%, at least 97%, at least 98%, or at least 99% identity thereto.

Embodiment III-122. The polynucleotide of any one of embodiments III-1 to III-121, comprising one or more sequences selected from the group consisting of the sequences of Tables 8-10, 12, 13, 17-22 and 24-27.

Embodiment III-123. The polynucleotide of any one of embodiments III-1 to III-122, comprising one or more sequences selected from the group consisting of the sequences of Table 26, or a sequence having at least 85%, at least 90%, at least 95%, at least 95%, at least 96%, at least 97%, at least 98%, or at least 99% identity thereto.

Embodiment III-124. The polynucleotide of any one of embodiments III-1 to III-123, comprising one or more sequences selected from the group consisting of the sequences of Table 26.

Embodiment III-125. The polynucleotide of embodiment III-124, comprising a sequence of a construct selected from the group of constructs of 1-174, 177-186, and 188-198 as set forth in Table 26.

Embodiment III-126. The polynucleotide of any one of embodiments III-123 to III-125, wherein the sequence further comprises a targeting sequence selected from the group of sequences of SEQ ID NOS: 41056-41776 as set forth in Table 27, wherein the targeting sequence is linked to the 3′ end of the polynucleotide sequence encoding the gRNA.

Embodiment III-127. The polynucleotide of any one of embodiments III-1 to III-126, wherein one or more AAV component sequences selected from the group consisting of 5′ ITR, 3′ ITR, pol III promoter, pol II promoter, encoding sequence for CRISPR nuclease, encoding sequence for gRNA, accessory element, and poly(A) are modified for depletion of all or a portion of the CpG dinucleotides of the sequences

Embodiment III-128. The polynucleotide of embodiment III-127, wherein one or more AAV component sequences selected from the group consisting of 5′ ITR, 3′ ITR, pol III promoter, pol II promoter, encoding sequence for a CRISPR nuclease, encoding sequence for gRNA, and poly(A), and accessory element comprise less than about 10%, less than about 5%, or less than about 1% CpG dinucleotides.

Embodiment III-129. The polynucleotide of embodiment III-127, wherein one or more AAV component sequences selected from the group consisting of 5′ ITR, 3′ ITR, pol III promoter, pol II promoter, encoding sequence for a CRISPR nuclease, encoding sequence for gRNA, and poly(A), and accessory element are devoid of CpG dinucleotides.

Embodiment III-130. The polynucleotide of any one of embodiment III-127 to III-129, wherein the one or more AAV component sequences codon-optimized for depletion of all or a portion of the CpG dinucleotides are selected from the group consisting of SEQ ID NOS: 41045-41055 as set forth in Table 25.

Embodiment III-131. The polynucleotide of any one of embodiments III-1 to III-130, wherein the polynucleotide has the configuration of a construct depicted in any one of FIG. 24, 33-35, or 42.

Embodiment III-132. A recombinant adeno-associated virus vector (rAAV) comprising: a) an AAV capsid protein, and b) the polynucleotide of any one of embodiments III-1 to III-131.

Embodiment III-133. The rAAV of embodiment III-132, wherein the AAV capsid protein is derived from serotype AAV1, AAV2, AAV3, AAV4, AAV5, AAV6, AAV7, AAV8, AAV9, AAV10, AAV 11, AAV12, AAV 44.9, AAV-Rh74, or AAVRh10.

Embodiment III-134. The rAAV of embodiment III-133, wherein the AAV capsid protein and the 5′ and 3′ ITR are derived from the same serotype of AAV.

Embodiment III-135. The rAAV of embodiment III-133, wherein the AAV capsid protein and the 5′ and 3′ ITR are derived from different serotypes of AAV.

Embodiment III-136. The rAAV of embodiment III-135, wherein the 5′ and 3′ ITR are derived from AAV serotype 2.

Embodiment III-137. The rAAV of any of embodiments III-132 to III-136, wherein upon transduction of a cell with the rAAV, the CRISPR protein and gRNA are capable of being expressed.

Embodiment III-138. The rAAV of embodiment III-137, wherein upon expression, the gRNA is capable of forming a ribonucleoprotein (RNP) complex with the CRISPR protein.

Embodiment III-139. The rAAV of embodiment III-137 or III-138, wherein the AAV polynucleotide component sequences modified for depletion of all or a portion of the CpG dinucleotides substantially retain their functional properties upon expression.

Embodiment III-140. The rAAV of embodiment III-137 or III-138, wherein the AAV polynucleotide component sequences modified for depletion of all or a portion of the CpG dinucleotides exhibit a lower potential for inducing an immune response compared to an rAAV wherein the AAV polynucleotide is not modified for depletion of the CpG dinucleotides.

Embodiment III-141. The rAAV of embodiment III-140, wherein the lower potential for inducing an immune response is exhibited in an in vitro mammalian cell assay designed to detect production of one or more markers of an inflammatory response selected from the group consisting of TLR9, interleukin-1 (IL-1), IL-6, IL-12, IL-18, tumor necrosis factor alpha (TNF-α), interferon gamma (IFNγ), and granulocyte-macrophage colony stimulating factor (GM-CSF).

Embodiment III-142. The rAAV of embodiment III-141, wherein the rAAV comprising the AAV polynucleotide component sequences modified for depletion of all or a portion of the CpG dinucleotides elicits reduced production of the one or more inflammatory markers of at least about 10%, at least about 20%, at least about 30%, at least about 40%, at least about 50%, at least about 60%, at least about 80%, or at least about 90% less compared to the comparable rAAV that is not CpG depleted.

Embodiment III-143. The rAAV of embodiment III-140, wherein administration of a dose of the rAAV comprising the AAV polynucleotide component sequences modified for depletion of all or a portion of the CpG dinucleotides to a subject elicits a reduced immune response compared to an administered dose of the comparable rAAV that is not CpG depleted.

Embodiment III-144. The rAAV of embodiment III-143, wherein the reduced immune response is a reduction of the production of anti-rAAV antibodies or a delayed-type hypersensitivity reaction to an rAAV component in the subject.

Embodiment III-145. The rAAV of embodiment III-143, wherein the reduced immune response is determined by the measurement of one or more inflammatory markers in the blood of the subject selected from the group consisting of TLR9, interleukin-1 (IL-1), IL-6, IL-12, IL-18, tumor necrosis factor alpha (TNF-α), interferon gamma (IFNγ), and granulocyte-macrophage colony stimulating factor (GM-CSF), wherein the one or more markers are reduced by at least about 10%, at least about 20%, at least about 30%, at least about 40%, at least about 50%, at least about 60%, at least about 80%, or at least about 90% compared to the comparable rAAV that is not CpG depleted.

Embodiment III-146. The rAAV of any one of embodiments III-143 to III-145, wherein the subject is selected from mouse, rat, pig, dog, and non-human primate.

Embodiment III-147. The rAAV of any one of embodiments III-143 to III-145, wherein the subject is human.

Embodiment III-148. A pharmaceutical composition, comprising the rAAV of any one of embodiment III-132 and a pharmaceutically acceptable carrier, diluent or excipient.

Embodiment III-149. A method for modifying a target nucleic acid in a population of mammalian cells, comprising contacting a plurality of the cells with an effective amount of the rAAV of any one of embodiments III-132 to III-147, wherein the target nucleic acid of a gene of the cells targeted by the expressed gRNA is modified by the expressed CRISPR protein.

Embodiment III-150. The method of embodiment III-149, wherein the gene of the cells comprises one or more mutations.

Embodiment III-151. The method of embodiment III-149 or III-150, wherein the modifying comprises introducing an insertion, deletion, substitution, duplication, or inversion of one or more nucleotides in the target nucleic acid of the cells of the population.

Embodiment III-152. The method of any one of embodiments III-149 to III-151, wherein the gene is knocked down or knocked out.

Embodiment III-153. The method of any one of embodiments III-149 to III-151, wherein the gene is modified such that a functional gene product can be expressed.

Embodiment III-154. A method of treating a disease in a subject caused by one or more mutations in a gene of the subject, comprising administering a therapeutically effective dose of the rAAV of any one of embodiments III-132 to III-145 to the subject.

Embodiment III-155. The method of embodiment III-149, wherein the rAAV is administered to a subject at a dose of at least about 1×10⁸ vector genomes (vg), at least about 1×10⁵ vector genomes/kg (vg/kg), at least about 1×10⁶ vg/kg, at least about 1×10⁷ vg/kg, at least about 1×10⁸ vg/kg, at least about 1×10⁹ vg/kg, at least about 1×10¹⁰ vg/kg, at least about 1×10¹¹ vg/kg, at least about 1×10¹² vg/kg, at least about 1×10¹³ vg/kg, at least about 1×10¹⁴ vg/kg, at least about 1×10¹⁵ vg/kg, or at least about 1×10¹⁶ vg/kg.

Embodiment III-156. The method of embodiment III-154, wherein the rAAV is administered to a subject at a dose of at least about 1×10⁵ vg/kg to about 1×10¹⁶ vg/kg, at least about 1×10⁶ vg/kg to about 1×10¹⁵ vg/kg, or at least about 1×10⁷ vg/kg to about 1×10¹⁴ vg/kg.

Embodiment III-157. The method of any one of embodiments III-154 to III-156, wherein the rAAV is administered to the subject by a route of administration selected from subcutaneous, intradermal, intraneural, intranodal, intramedullary, intramuscular, intralumbar, intrathecal, subarachnoid, intraventricular, intracapsular, intravenous, intralymphatical, intraocular or intraperitoneal routes, and wherein the administering method is injection, transfusion, or implantation.

Embodiment III-158. The method of any one of embodiments III-149 to III-157, wherein the subject is selected from the group consisting of mouse, rat, pig, and non-human primate.

Embodiment III-159. The method of any one of embodiments III-149 to III-157, wherein the subject is a human.

Embodiment III-160. A method of making an rAAV vector, comprising:
a. providing a population of packaging cells; and
b. transfecting the population of cells with:
   i) a vector comprising the polynucleotide of any one of embodiments III-1 to III-131;
   ii) a vector comprising an aap (assembly) gene; and
   iii) a vector comprising rep and cap genomes.

Embodiment III-161. The method of embodiment III-160, wherein the packaging cell is selected from the group consisting of BHK cells, HEK293 cells, HEK293T cells, NS0 cells, SP2/0 cells, YO myeloma cells, P3X63 mouse myeloma cells, PER cells, PER.C6 cells, hybridoma cells, NIH3T3 cells, COS cells, HeLa cells, and CHO cells.

Embodiment III-162. The method of embodiment III-160 or III-161, the method further comprising recovering the rAAV vector.

Embodiment III-163. The method of any one of embodiments III-160 to III-162, wherein the component sequences of the AAV polynucleotide are encompassed in a single rAAV particle.

Embodiment III-164. A method of reducing the immunogenicity of an rAAV, comprising deleting all or a portion of the CpG dinucleotides of the sequences of the AAV component sequences selected from the group consisting of 5′ ITR, 3′ ITR, pol III promoter, pol II promoter, encoding sequence for CRISPR nuclease, encoding sequence for gRNA, accessory element, and poly(A).

Embodiment III-165. The method of embodiment III-164, wherein the one or more AAV polynucleotide component sequences comprise less than about 10%, less than about 5%, or less than about 1% CpG dinucleotides.

Embodiment III-166. The method of embodiment III-165, wherein one or more AAV polynucleotide component sequences are devoid of CpG dinucleotides.

Embodiment III-167. The method of any one of embodiment III-164 to III-166, wherein the one or more AAV polynucleotide component sequences are selected from the group consisting of SEQ ID NOS: 41045-41055 as set forth in Table 25.

Embodiment III-168. The method of any one of embodiments III-164 to III-167, wherein the rAAV exhibits a lower potential for inducing production of one or more markers of an inflammatory response in an in vitro mammalian cell assay compared to a comparable rAAV wherein the CpG dinucleotides have not been deleted, wherein the one or more inflammatory markers are selected from the group consisting of TLR9, interleukin-1 (IL-1), IL-6, IL-12, IL-18, tumor necrosis factor alpha (TNF-α), interferon gamma (IFNγ), and granulocyte-macrophage colony stimulating factor (GM-CSF).

Embodiment III-169. The method of embodiment III-168, wherein the rAAV elicits reduced production of the one or more inflammatory markers of at least about 10%, at least about 20%, at least about 30%, at least about 40%, at least about 50%, at least about 60%, at least about 80%, or at least about 90% less compared to the comparable rAAV that is not CpG depleted.

Embodiment III-170. The method of any one of embodiments III-164 to III-167, wherein administration of a dose of the rAAV comprising the AAV polynucleotide component sequences modified for depletion of all or a portion of the CpG dinucleotides to a subject elicits a reduced immune response compared to an administered dose of the comparable rAAV that is not CpG depleted.

Embodiment III-171. The method of embodiment III-170, wherein the reduced immune response is a reduction of the production of anti-rAAV antibodies or a delayed-type hypersensitivity reaction to an rAAV component in the subject.

Embodiment III-172. The method of embodiment III-170, wherein the reduced immune response is determined by the measurement of one or more inflammatory markers in the blood of the subject selected from the group consisting of TLR9, interleukin-1 (IL-1), IL-6, IL-12, IL-18, tumor necrosis factor alpha (TNF-α), interferon gamma (IFNγ), and granulocyte-macrophage colony stimulating factor (GM-CSF), wherein the one or more markers are reduced by at least about 10%, at least about 20%, at least about 30%, at least about 40%, at least about 50%, at least about 60%, at least about 80%, or at least about 90% compared to the comparable rAAV that is not CpG depleted.

Embodiment III-173. The method of any one of embodiments III-164 to III-172, wherein the subject is selected from mouse, rat, pig, dog, and non-human primate.

Embodiment III-174. The method of any one of embodiments III-164 to III-172, wherein the subject is human.

Embodiment III-175. A composition of an rAAV of any one of embodiments III-132 to III-147, for use as a medicament for the treatment of a human in need thereof.

The present description sets forth numerous exemplary configurations, methods, parameters, and the like. It should be recognized, however, that such description is not intended as a limitation on the scope of the present disclosure, but is instead provided as a description of exemplary embodiments. The following examples are included for illustrative purposes and are not intended to limit the scope of the invention.

### EXAMPLES

### Example 1: Small Class 2, Type V CRISPR Proteins can Edit the Genome when Expressed from an AAV Episome In Vitro

Experiments were conducted to demonstrate that small Class 2, Type V CRISPR proteins can edit a genome when expressed from an AAV plasmid or an AAV vector in vitro.

### Materials and Methods:

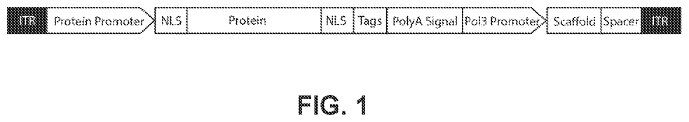

The AAV transgene was conceptually broken up between ITRs into different parts, which consisted of the therapeutic cargo and accessory elements relevant to expression of the therapeutic cargo in mammalian cells. AAV vectorology consisted of identifying a parts list and subsequently designing, building, and testing vectors in both plasmid and AAV form in mammalian cells. A schematic and one configuration of its components is shown in FIG. 1.

In this first example, three plasmids were constructed (construct 1, construct 2, and construct 3; see Table 26 for component sequences), where the only difference in the plasmid sequence between the ITRs was in the affinity tag region.

### Cloning and QC:

AAV vectors were cloned using a 4-part Golden Gate Assembly consisting of a pre-digested AAV backbone, small CRISPR protein-encoding DNA, and flanking 5′ and 3′ DNA sequences. 5′ sequences contained enhancer, protein promoter and N-terminal NLS, while 3′ sequences contained C-terminal NLS, WPRE, poly(A) signal, RNA promoter and guide RNA containing spacer 12.7, targeting tDTomato (DNA sequence: CTGCATTCTAGTTGTGGTTT (SEQ ID NO: 40800)). 5′ and 3′ parts were ordered as gene fragments from Twist, PCR-amplified, and assembled into AAV vectors through cyclical Golden Gate reactions using T4 Ligase and BbsI.

Assembled AAV vectors were then transformed into chemically-competent E. coli (Stbl3s). Transformed cells were recovered for 1 hour in a 37° C. shaking incubator, plated on Kanamycin LB-Agar plates and allowed to grow at 37° C. for 12-16 hours. Colony PCR was performed to determine clones that contained full transgenes. Correct clones were inoculated in 50 mL of LB media with kanamycin and grown overnight. Plasmids were then midiprepped the following day and sequence-verified. To assess the quality of midipreps, constructs were processed in restriction digests with XmaI (which cuts in each of the ITRs) and XhoI (which cuts once in the AAV genome). Digests and uncut constructs were then run on a 1% agarose gel and imaged on a ChemiDoc. If the plasmid was >90% supercoiled, the correct size, and the ITRs were intact, the construct was tested via nucleofection and/or transduction

### Method for Plasmid Nucleofection:

Plasmids containing the AAV genome were transfected in a mouse immortalized neural progenitor cell (NPC) line isolated from the Ai9-tdTomato mouse (tdTomato mNPCs) using the Lonza P3 Primary Cell 96-well Nucleofector Kit. Briefly, Ai9 is a Cre reporter tool strain designed to have a loxP flanked STOP cassette preventing the transcription of a CAG promoter-driven tdTomato marker. Ai9 mice, or Ai9 mNPCs, express tdTomato following Cre-mediated recombination to remove the STOP cassette. Sequence-validated plasmids were diluted to concentrations of 200 ng/μl, 100 ng/μl, 50 ng/μL and 25 ng/μL, and 5 μL of each (1000 ng, 500 ng, 250 ng and 125 ng) were added to P3 solution containing 200,000 tdTomato mNPCs. The combined solution was nucleofected using a Lonza 4D Nucleofector System following program EH-100. Following nucleofection, the solution was quenched with pre-equilibrated mNPC medium (DMEM/F12 with GlutaMax, 10 mM HEPES, 1×MEM Non-Essential Amino Acids, 1× penicillin/streptomycin, 1:1000 2-mercaptoethanol, 1× B-27 supplement, minus vitamin A, 1× N2 with supplemented growth factors bFGF and EGF (20 ng/mL final concentration). The solution was then aliquoted in triplicate (approx. 67,000 cells per well) in a 96-well plate coated with PLF (1× Poly-DL-ornithine hydrobromide, 10 mg/mL in sterile diH20, 1× laminin, and 1× fibronectin). 48 hours after transfection, treated cells were replenished with fresh mNPC media containing growth factors. 5 days after transfection, tdTomato mNPCs were lifted and activity was assessed by FACS.

### Aav Production:

Suspension HEK293T cells were adapted from parental HEK293T and grown in FreeStyle 293 media. For screening purposes, small scale cultures (20-30 mL cultured in 125 mL Erlenmeyer flasks and agitated at 110 rpm) were diluted to a density of 1.5e+6 cells/mL on the day of transfection. Endotoxin-free pAAV plasmids with the transgene flanked by ITR repeats were co-transfected with plasmids supplying the adenoviral helper genes for replication and AAV rep/cap genome using PEIMax (Polysciences) in serum-free OPTIMEM media. Cultures were supplemented with 10% CDM4HEK293 (HyClone) 3 hours post-transfection. Three days later, cultures were centrifuged at 1000 rpm for 10 minutes to separate the supernatant from the cell pellet. The supernatant was mixed with 40% PEG 2.5M NaCl (8% final concentration) and incubated on ice for at least 2 hours to precipitate AAV viral particles. The cell pellet, containing the majority of the AAV vectors, was resuspended in lysis media (0.15 M NaCl, 50 mM Tris HCl, 0.05% Tween, pH 8.5), sonicated on ice (15 seconds, 30% amplitude) and treated with Benzonase (250 U/μL, Novagen) for 30 minutes at 37° C. Crude lysate and PEG-treated supernatant were then centrifuged at 4000 rpm for 20 minutes at 4° C. to resuspend the PEG precipitated AAV (pellet) with cell debris-free crude lysate (supernatant), and then clarified further using a 0.45 μM filter.

To determine the viral genome titer, 1 μL from crude lysate viruses was digested with DNase and ProtK, followed by quantitative PCR. 5 μL of digested virus was used in a 25 μL qPCR reaction composed of IDT primetime master mix and a set of primer and 6′FAM/Zen/IBFQ probe (IDT) designed to amplify the CMV promoter region (Fwd 5′-CATCTACGTATTAGTCATCGCTATTACCA-3′ (SEQ ID NO: 40801)); Rev 5′-GAAATCCCCGTGAGTCAAACC-3′ (SEQ ID NO: 40802)), Probe 5′-TCAATGGGCGTGGATAG-3′ (SEQ ID NO: 40803)) or a 62 nucleotide-fragment located in the AAV2-ITR (Fwd 5′-GGAACCCCTAGTGATGGAGTT-3′ (SEQ ID NO: 40804); Rev 5′-CGGCCTCAGTGAGCGA-3′ (SEQ ID NO: 40805), Probe 5′-CACTCCCTCTCTGCGCGCTCG-3′). Ten-fold serial dilutions (5 μl each of 2e+9 to 2e+4 DNA copies/mL) of an AAV ITR plasmid was used as reference standards to calculate the titer (viral genome (vg)/mL) of viral samples. QPCR program was set up as: initial denaturation step at 95° ° C. for 5 minutes, followed by 40 cycles of denaturation at 95° C. for 1 min and annealing/extension at 60° C. for 1 min.

### Aav Transduction:

10,000 cells/well of mNPCs were seeded on PLF-coated wells in 96-well plates 48-hours before AAV transduction. All viral infection conditions were performed in triplicate, with normalized number of vg among experimental vectors, in a series of 3-fold dilution of multiplicity of infection (MOI) ranging from ˜1.0e+6 to 1.0e+4 vg/cell. Final volumes of 50 μL of AAV vectors diluted in pre-equilibrated mNPC medium supplemented with bFGF/EGF growth factors (20 ng/ml final concentration) were applied to each well. 48 hours post-transfection, complete media change was performed with fresh media supplemented with growth factors. Editing activity (tdT+ cell quantification) was assessed by FACS 5 days post-transfection.

### Method for Assessing Activity by FACS:

Five days after transfection, treated tdTomato mNPCs in 96-well plates were washed with dPBS and treated with 50 μL TrypLE for 15 minutes. Following cell dissociation, treated wells were quenched with media containing DMEM, 10% FBS and 1× penicillin/streptomycin. Resuspended cells were transferred to round-bottom 96-well plates and centrifuged for 5 min at 1000× g. Cell pellets were then resuspended with dPBS containing 1×DAPI, and plates were loaded into an Attune NxT Flow Cytometer Autosampler. The Attune NxT flow cytometer was run using the following gating parameters: FSC-A×SSC-A to select cells, FSC-H×FSC-A to select single cells, FSC-A× VL1-A to select DAPI-negative alive cells, and FSC-A× YL1-A to select tdTomato positive cells.

### Results:

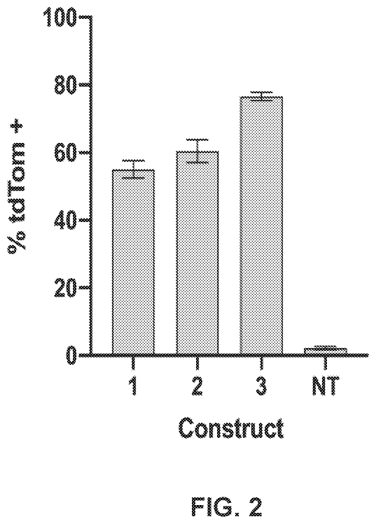

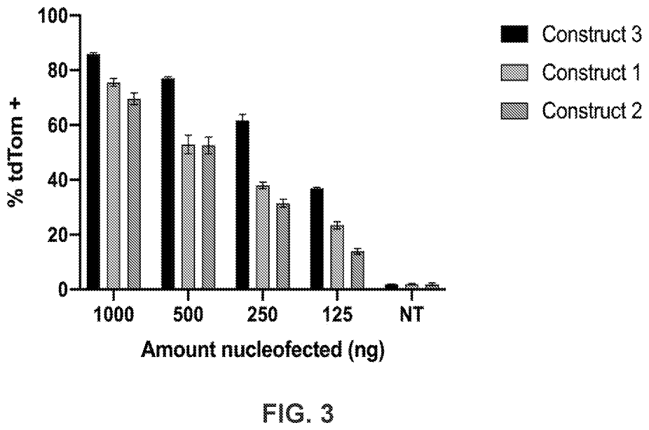

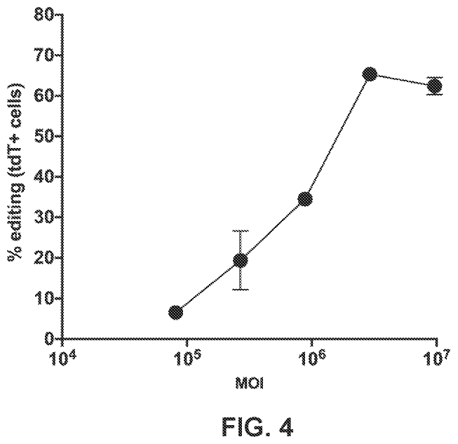

The graph in FIG. 2 shows that CasX variant 491 and guide variant 174 with spacer 12.7 targeting the tdTomato stop cassette, when delivered by nucleofection of an AAV transgene plasmid, was able to edit the target stop cassette in mNPCs (measured by percentage of cells that are tdTom+ by FACS). Among the vectors tested, CasX 491.174 delivered in construct 3 (with 80% tdTomato+ cells) outperformed the others. FIG. 3 shows that all three vectors tested achieved editing at the tdTomato locus in a dose-dependent manner. FIG. 4 shows results of editing using construct 3 in an AAV vector, which demonstrated a dose-dependent response, achieving a high degree of editing.

The experiments demonstrate that small Class 2, Type V CRISPR proteins (such as CasX) and targeted guides can edit the genome when expressed from an AAV transgene plasmid or episome in vitro.

### Example 2: Packaging of Small Class 2, Type V CRISPR Systems within an AAV Vector

Experiments were conducted to demonstrate that systems of small Class 2, Type V CRISPR proteins such as CasX and gRNA can be encoded and efficiently packaged within a single AAV vector.

### Materials and Methods:

For this experiment, AAV vectors were generated with transgenes packaging CasX variant 438, gRNA scaffold 174 and spacer 12.7 using the methods for AAV production, purification and characterization, as described in Example 1. For characterization, AAV viral genomes were titered by qPCR, and the empty-full ratio was quantified using scanning transmission electron microscopy (STEM). The AAV were negatively stained with 1% uranyl acetate and visualized. Empty particles were identified by presence of a dark electron dense circle at the center of the capsid.

### Results:

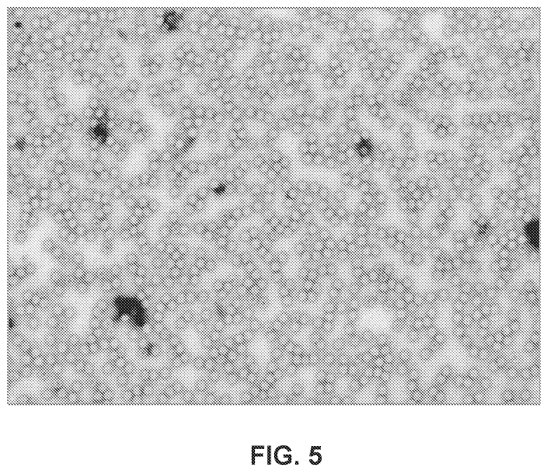

The genomic DNA titers (by qPCR) for the AAV preparation was measured to be 6e12 vg/mL, generated from 1 L of HEK293T cell culture. FIG. 5 is an image from a scanning transmission electron microscopy (STEM) micrograph showing that an estimated 90% of the particles in this AAV formulation contained viral genomes; e.g., were full. Under the conditions of the experiment, the results demonstrate that CasX variant proteins and gRNA can be efficiently packaged in single AAV vector particles, resulting in high titers and high packaging efficiency.

### Example 3: In Vivo Editing of a Genome with Small Class 2, Type V CRISPR Proteins Expressed from an AAV Episome

Experiments were conducted to demonstrate that small Class 2, Type V CRISPR proteins, such as CasX, are capable of editing the genome when expressed from an AAV episome in vivo.

### Materials and Methods:

For this experiment, AAV vectors were generated using the methods for AAV production, purification and characterization, as described in Example 1.

In vivo AAV administration and tissue processing:

P0-P1 pups from Ai9 mice were injected with AAV with a transgene encoding CasX variant 491 and guide variant 174 with spacer 12.7. Briefly, mice were cryo-anesthetized and 1-2 μL of AAV vector (˜1e11 viral genomes (vg)) was unilaterally injected into the intracerebroventricular (ICV) space using a Hamilton syringe (10 μL, Model 1701 RN SYR Cat No: 7653-01) fitted with a 33-gauge needle (small hub RN NDL—custom length 0.5 inches, point 4 (45 degrees)). Post-injection, pups were recovered on a warm heating pad before being returned to their cages. 1 month after ICV injections, animals were terminally anesthetized with an intraperitoneal injection of ketamine/xylazine, and perfused transcardially with saline and fixative (4% paraformaldehyde). Brains were dissected and further post-fixed in 4% PFA, followed by infiltration with 30% sucrose solution, and embedding in OCT compound. OCT-embedded brains were coronally sectioned using a cryostat. Sections were then mounted on slides, counter-stained with DAPI to label cell nuclei, coverslipped and imaged on a fluorescence microscope. Images were processed using ImageJ software and editing levels were quantified by counting the number of tdTom+ cells as a percentage of DAPI-labeled nuclei.

In a subsequent experiment to assess editing in peripheral tissues, particularly in the liver and in the heart, P0-P1 pups from Ai9 mice were cryo-anesthetized and were intravenously injected with ˜1e12 viral genomes (vg) of the same AAV construct in a 40 μL volume. Post-injection, pups were recovered on a warm heating pad before being returned to their cages. 1 month post-administration, animals were terminally anesthetized and heart and liver tissues were necropsied and processed as described above.

### Results:

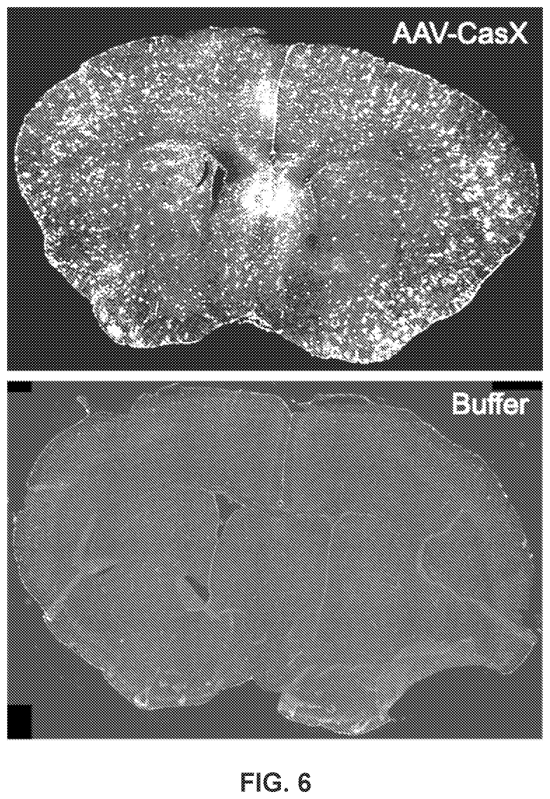

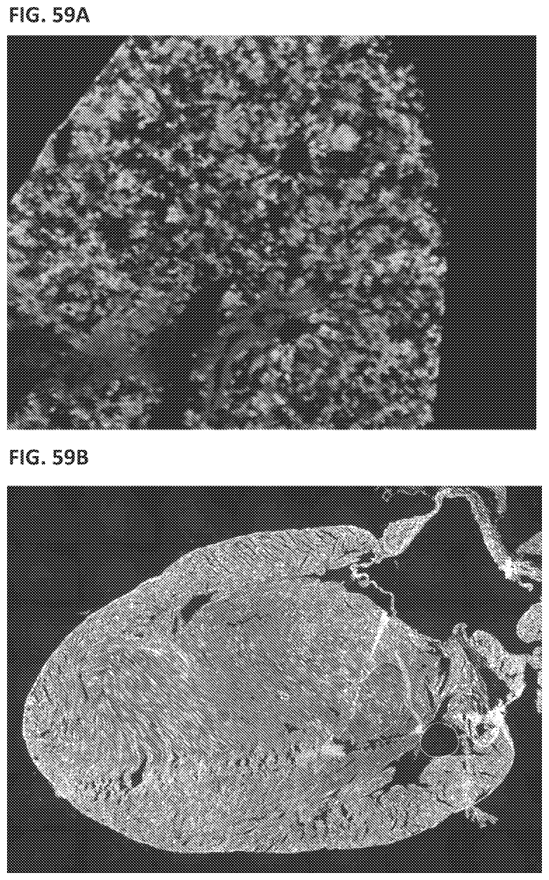

FIG. 6 provides comparative immunohistochemistry (IHC) images of brain tissue processed from an Ai9 mouse that received an ICV injection of AAV packaging CasX variant 491 and guide scaffold 174 with spacer 12.7 (top) against an ICV injection of AAV packaging CasX variant 491 and guide 174 with spacer 12.7 and stained with 4′,6-diamidino-2-phenylindole. The signal from cells in the tdTom channel indicates that the tdTom locus within these cells was successfully edited. The tdTom+ cells (in white) are distributed evenly across all regions of the brain, indicating that ICV-administered AAV packaging the encoded CasX, guide and spacer are able to reach and edit these cells (top panel) as compared to a non-targeting control (bottom panel). The FIG. 6 images are representative of those obtained from 3 mice for each group. Additionally, the results presented in FIG. 59A (liver) and 59B (heart) demonstrate that the AAV were able to distribute within the liver and the heart (edited cells in white) and edit the genome when expressed from single AAV episomes in vivo.

The results demonstrate that that AAV encoding small CRISPR proteins (such as CasX) and a targeting guide can distribute within the tissues, when delivered either locally (brain) or systemically, and edit the genome when expressed from single AAV episomes in vivo.

### Example 4: Small CRISPR Protein Potency is Enhanced by AAV Vector Protein Promoter Choice

Experiments were conducted to demonstrate that small CRISPR protein expression, such as CasX, can be enhanced by utilizing different promoters in an AAV construct for the encoded protein. Cargo space in the AAV transgene can be maximized with the use of short promoters in combination with CasX. Additionally, experiments were conducted to demonstrate that expression can be enhanced with the use of promoters that would otherwise be too long to be efficiently packaged in AAV vector, if they were combined with larger CRISPR proteins, such as Cas9. The use of long, cell-type-specific promoters to enhance small CRISPR proteins is an advantage to the AAV system described herein, and not possible in traditional CRISPR systems due to the size of other CRISPR proteins.

### Materials and Methods:

Cloning and QC were conducted as described in Example 1. Promoter variants were cloned upstream of CasX protein in an AAV-cis plasmid. The sequences of the additional components of the AAV constructs, with the exception of sequences encoding the CasX (Table 21) and the one or more gRNA (Tables 18 and 19), are listed in Table 26.

TABLE 8
Promoter variant sequences

| SEQ ID | Construct | Promoter | | Size |
|---|---|---|---|---|
| NO: | ID | based on | Sequence | (bp) |
| 40370 | 1, 2, 3, | CMV | GACATTGATTATTGACTAGTTATTAATAGTAATCAATTAC | 584 |
| | 7, 44 | | GGGGTCATTAGTTCATAGCCCATATATGGAGTTCCGCGT | |
| | | | TACATAACTTACGGTAAATGGCCCGCCTGGCTGACCGCC | |
| | | | CAACGACCCCCGCCCATTGACGTCAATAATGACGTATGT | |
| | | | TCCCATAGTAACGCCAATAGGGACTTTCCATTGACGTCA | |
| | | | ATGGGTGGAGTATTTACGGTAAACTGCCCACTTGGCAGT | |
| | | | ACATCAAGTGTATCATATGCCAAGTACGCCCCCTATTGA | |
| | | | CGTCAATGACGGTAAATGGCCCGCCTGGCATTATGCCCA | |
| | | | GTACATGACCTTATGGGACTTTCCTACTTGGCAGTACATC | |
| | | | TACGTATTAGTCATCGCTATTACCATGGTGATGCGGTTTT | |
| | | | GGCAGTACATCAATGGGCGTGGATAGCGGTTTGACTCAC | |
| | | | GGGGATTTCCAAGTCTCCACCCCATTGACGTCAATGGGA | |
| | | | GTTTGTTTTGGCACCAAAATCAACGGGACTTTCCAAAAT | |
| | | | GTCGTAACAACTCCGCCCCATTGACGCAAATGGGCGGTA | |
| | | | GGCGTGTACGGTGGGAGGTCTATATAAGCAGAGCT | |
| 40371 | 4 | UbC | GGCCTCCGCGCCGGGTTTTGGCGCCTCCCGCGGGCGCCCC | 400 |
| | | | CCTCCTCACGGCGAGCGCTGCCACGTCAGACGAAGGGCG | |
| | | | CAGCGAGCGTCCTGATCCTTCCGCCCGGACGCTCAGGAC | |
| | | | AGCGGCCCGCTGCTCATAAGACTCGGCCTTAGAACCCCA | |
| | | | GTATCAGCAGAAGGACATTTTAGGACGGGACTTGGGTGA | |
| | | | CTCTAGGGCACTGGTTTTCTTTCCAGAGAGCGGAACAGGC | |
| | | | GAGGAAAAGTAGTCCCTTCTCGGCGATTCTGCGGAGGGA | |
| | | | TCTCCGTGGGGCGGTGAACGCCGATGATTATATAAGGAC | |
| | | | GCGCCGGGTGTGGCACAGCTAGTTCCGTCGCAGCCGGGA | |
| | | | TTTGGGTCGCGGTTCTTGTTTGTGGATCGCTGTGATCGTC | |
| | | | ACTTGGT | |
| 40372 | 5 | EFS | TGGCTCCGGTGCCCGTCAGTGGGCAGAGCGCACATCGCC | 234 |
| | | | CACAGTCCCCGAGAAGTTGGGGGGAGGGGTCGGCAATTG | |
| | | | AACCGGTGCCTAGAGAAGGTGGCGCGGGGTAAACTGGGA | |
| | | | AAGTGATGTCGTGTACTGGCTCCGCCTTTTTCCCGAGGGT | |
| | | | GGGGGAGAACCGTATATAAGTGCAGTAGTCGCCGTGAAC | |
| | | | GTTCTTTTTCGCAACGGGTTTGCCGCCAGAACACAGGT | |
| 40373 | 6 | CMV-s | CGTTACATAACTTACGGTAAATGGCCCGCCTGGCTGACCG | 335 |
| | | | CCCAACGACCCCCGCCCATTGACGTCAATAATGACGTAT | |
| | | | GTTCCCATAGTAACGCCAATAGGGACTTTCCATTGACGTC | |
| | | | AATGGGTGGAGTATTTACGGTAAACTGCCCACTTGGCAG | |
| | | | TACATCAAGTGTATCATATGCCAAGTACGCCCCCTATTGA | |
| | | | CGTCAATGACGGTAAATGGCCCGCCTGGCATTATGCCCA | |
| | | | GTACATGACCTTATGGGACTTTCCTACTTGGCAGTACATC | |
| | | | TACGTATTAGTCATCGCTATTACCATGAGAGGGTATATAA | |
| | | | TGGAAGCTCGACTTCCAG | |
| 40374 | 8 | CMVd1 | CCAAAATCAACGGGACTTTCCAAAATGTCGTAATAACCC | 100 |
| | | | CGCCCCGTTGACGCAAATGGGCGGTAGGCGTGTACGGTG | |
| | | | GGAGGTCTATATAAGCAGAGCT | |
| 40375 | 9 | CMVd2 | GACGCAAATGGGCGGTAGGCGTGTACGGTGGGAGGTCTA | 52 |
| | | | TATAAGCAGAGCT | |
| 40376 | 10 | miniCMV | GGTAGGCGTGTACGGTGGGAGGTCTATATAAGCAGAGCT | 39 |
| 40377 | 11, 26 | HSVTK | ATGACACAAACCCCGCCCAGCGTCTTGTCATTGGCGAATT | 146 |
| | | | CGAACACGCAGATGCAGTCGGGGCGGCGCGGTCCCAGGT | |
| | | | CCACTTCGCATATTAAGGTGACGCGTGTGGCCTCGAACAC | |
| | | | CGAGCGACCCTGCAGCGACCCGCTTAA | |
| 40378 | 12 | miniTK | TTCGCATATTAAGGTGACGCGTGTGGCCTCGAACACCGA | 63 |
| | | | GCGACCCTGCAGCGACCCGCTTAA | |
| 40379 | 13 | miniIL2 | CATTTTGACACCCCCATAATATTTTTCCAGAATTAACAGT | 114 |
| | | | ATAAATTGCATCTCTTGTTCAAGAGTTCCCTATCACTCTC | |
| | | | TTTAATCACTACTCACAGTAACCTCAACTCCTGC | |
| 40380 | 14 | GRP94 | ACTAGTTTCATCACCACCGCCACCCCCCCGCCCCCCCGC | 710 |
| | | | CATCTGAAAGGGTTCTAGGGGATTTGCAACCTCTCTCGT | |
| | | | GTGTTTCTTCTTTCCGAGAAGCGCCGCCACACGAGAAAG | |
| | | | CTGGCCGCGAAAGTCGTGCTGGAATCACTTCCAACGAAA | |
| | | | CCCCAGGCATAGATGGGAAAGGGTGAAGAACACGTTGC | |
| | | | CATGGCTACCGTTTCCCCGGTCACGGAATAAACGCTCTC | |
| | | | TAGGATCCGGAAGTAGTTCCGCCGCGACCTCTCTAAAAG | |
| | | | GATGGATGTGTTCTCTGCTTACATTCATTGGACGTTTTCC | |
| | | | CTTAGAGGCCAAGGCCGCCCAGGCAAAGGGGCGGTCCC | |
| | | | ACGCGTGAGGGGCCCGCGGAGCCATTTGATTGGAGAAA | |
| | | | AGCTGCAAACCCTGACCAATCGGAAGGAGCCACGCTTCG | |
| | | | GGCATCGGTCACCGCACCTGGACAGCTCCGATTGGTGGA | |
| | | | CTTCCGCCCCCCCTCACGAATCCTCATTGGGTGCCGTGG | |
| | | | GTGCGTGGTGCGGCGCGATTGGTGGGTTCATGTTTCCCG | |
| | | | TCCCCCGCCCGCGAGAAGTGGGGGTGAAAAGCGGCCCG | |
| | | | ACCTGCTTGGGGTGTAGTGGGCGGACCGCGCGGCTGGAG | |
| | | | GTGTGAGGATCCGAACCCAGGGGTGGGGGGTGGAGGCG | |
| | | | GCTCCTGCGATCGAAGGGGACTTGAGACT | |
| | | | CACCGGCCGCACGTC | |
| 40381 | 15 | Supercore 1 | GTACTTATATAAGGGGGTGGGGGCGCGTTCGTCCTCAGT | 81 |
| | | | CGCGATCGAACACTCGAGCCGAGCAGACGTGCCTACGG | |
| | | | ACCG | |
| 40382 | 16 | Supercore 2 | AGGTCTATATAAGCAGAGCTCGTTTAGTGAACCGTCAGA | 81 |
| | | | TCGCCTGGAGACGTCGAGCCGAGTGGTTGTGCCTCCATA | |
| | | | GAA | |
| 40383 | 17 | Supercore 3 | AGGTCTATATAAGCAGAGCTCGTTTAGTGAACCGTCAGT | 81 |
| | | | CCGCCTGGAGACCTGCAGCCGAGTGGTCGTGCCTCCATA | |
| | | | GAA | |
| 40384 | 18 | Mecp2 | AGCTGAATGGGGTCCGCCTCTTTTCCCTGCCTAAACAGA | 229 |
| | | | CAGGAACTCCTGCCAATTGAGGGCGTCACCGCTAAGGCT | |
| | | | CCGCCCCAGCCTGGGCTCCACAACCAATGAAGGGTAATC | |
| | | | TCGACAAAGAGCAAGGGGTGGGGCGCGGGCGCGCAGGT | |
| | | | GCAGCAGCACACAGGCTGGTCGGGAGGGCGGGGCGCGA | |
| | | | CGTCTGCCGTGCGGGGTCCCGGCATCGGTTGCGCGC | |
| 40385 | 19 | CMVmini | GGTAGGCGTGTACGGTGGGAGGCCTATATAAGCAGAGC | 68 |
| | | | TCGTTTAGTGAACCGTCAGATCGCCTGGAG | |
| 40386 | 20 | CMVmini2 | AGGTCTATATAAGCAGAGCTCTCTGGCTAACTAGAGAAC | 65 |
| | | | CCACTGCTTAACTGGCTTATCGAAAT | |
| 40387 | 21 | miniCMVIE | GGTAGGCGTGTACGGTGGGAGGTCTATATAAGCAGAGCT | 39 |
| 40388 | 22 | adML | GGGGCTATAAAAGGGGGTGGGGGCGCGTTCGTCCTCACT | 81 |
| | | | CTCTTCCGCATCGCTGTCTGCGAGGGCCAGCTGTTGGGG | |
| | | | TGA | |
| 40389 | 23 | hepB | GGGGGAGGAGATTAGGTTAAAGGTCTTTGTATTAGGAGG | 107 |
| | | | CTGTAGGCATAAATTGGTCTGTTCACCAGCACCATGCAA | |
| | | | CTTTTTCACCTCTGCCTAATCATCTCATG | |
| 40390 | 54 | RSV | TGTAGTCTTATGCAATACTCTTGTAGTCTTGCAACATGGT | 227 |
| | | | AACGATGAGTTAGCAACATGCCTTACAAGGAGAGAAAAA | |
| | | | GCACCGTGCATGCCGATTGGTGGAAGTAAGGTGGTACGA | |
| | | | TCGTGCCTTATTAGGAAGGCAACAGACGGGTCTGACATG | |
| | | | GATTGGACGAACCACTGAATTGCCGCATTGCAGAGATAT | |
| | | | TGTATTTAAGTGCCTAGCTCGATACATAAAC | |
| 40391 | 55 | hSyn | AGTGCAAGTGGGTTTTAGGACCAGGATGAGGCGGGGTGG | 448 |
| | | | GGGTGCCTACCTGACGACCGACCCCGACCCACTGGACAA | |
| | | | GCACCCAACCCCCATTCCCCAAATTGCGCATCCCCTATCA | |
| | | | GAGAGGGGGAGGGGAAACAGGATGCGGCGAGGCGCGTG | |
| | | | CGCACTGCCAGCTTCAGCACCGCGGACAGTGCCTTCGCCC | |
| | | | CCGCCTGGCGGCGCGCGCCACCGCCGCCTCAGCACTGAA | |
| | | | GGCGCGCTGACGTCACTCGCCGGTCCCCCGCAAACTCCCC | |
| | | | TTCCCGGCCACCTTGGTCGCGTCCGCGCCGCCGCCGGCCC | |
| | | | AGCCGGACCGCACCACGCGAGGCGCGAGATAGGGGGGC | |
| | | | ACGGGCGCGACCATCTGCGCTGCGGCGCCGGCGACTCAG | |
| | | | CGCTGCCTCAGTCTGCGGTGGGCAGCGGAGGAGTCGTGT | |
| | | | CGTGCCTGAGAGCGCAG | |
| 40392 | 56 | SV40 | GTGTGTCAGTTAGGGTGTGGAAAGTCCCCAGGCTCCCCA | 330 |
| | | | GCAGGCAGAAGTATGCAAAGCATGCATCTCAATTAGTCA | |
| | | | GCAACCAGGTGTGGAAAGTCCCCAGGCTCCCCAGCAGGC | |
| | | | AGAAGTATGCAAAGCATGCATCTCAATTAGTCAGCAACC | |
| | | | ATAGTCCCGCCCCTAACTCCGCCCATCCCGCCCCTAACTC | |
| | | | CGCCCAGTTCCGCCCATTCTCCGCCCCATGGCTGACTAAT | |
| | | | TTTTTTTATTTATGCAGAGGCCGAGGCCGCCTCGGCCTCT | |
| | | | GAGCTATTCCAGAAGTAGTGAGGAGGCTTTTTTGGAGGC | |
| | | | CTAGGCTTTTGCAAA | |
| 40393 | 57 | hPGK | GGGGTTGGGGTTGCGCCTTTTCCAAGGCAGCCCTGGGTTT | 551 |
| | | | GCGCAGGGACGCGGCTGCTCTGGGCGTGGTTCCGGGAAA | |
| | | | CGCAGCGGCGCCGACCCTGGGTCTCGCACATTCTTCACGT | |
| | | | CCGTTCGCAGCGTCACCCGGATCTTCGCCGCTACCCTTGT | |
| | | | GGGCCCCCCGGCGACGCTTCCTGCTCCGCCCCTAAGTCGG | |
| | | | GAAGGTTCCTTGCGGTTCGCGGCGTGCCGGACGTGACAA | |
| | | | ACGGAAGCCGCACGTCTCACTAGTACCCTCGCAGACGGA | |
| | | | CAGCGCCAGGGAGCAATGGCAGCGCGCCGACCGCGATGG | |
| | | | GCTGTGGCCAATAGCGGCTGCTCAGCAGGGCGCGCCGAG | |
| | | | AGCAGCGGCCGGGAAGGGGCGGTGCGGGAGGCGGGGTG | |
| | | | TGGGGCGGTAGTGTGGGCCCTGTTCCTGCCCGCGCGGTGT | |
| | | | TCCGCATTCTGCAAGCCTCCGGAGCGCACGTCGGCAGTC | |
| | | | GGCTCCCTCGTTGACCGAATCACCGACCTCTCTCCCCAG | |
| 40394 | 58 | Jet | GGGCGGAGTTAGGGCGGAGCCAATCAGCGTGCGCCGTTC | 164 |
| | | | CGAAAGTTGCCTTTTATGGCTGGGCGGAGAATGGGCGGT | |
| | | | GAACGCCGATGATTATATAAGGACGCGCCGGGTGTGGCA | |
| | | | CAGCTAGTTCCGTCGCAGCCGGGATTTGGGTCGCGGTTCT | |
| | | | TGTTTGT | |
| 40395 | 59 | Jet + UsP | GGGCGGAGTTAGGGCGGAGCCAATCAGCGTGCGCCGTTC | 326 |
| | | intron | CGAAAGTTGCCTTTTATGGCTGGGCGGAGAATGGGCGGT | |
| | | | GAACGCCGATGATTATATAAGGACGCGCCGGGTGTGGCA | |
| | | | CAGCTAGTTCCGTCGCAGCCGGGATTTGGGTCGCGGTTCT | |
| | | | TGTTTGTGGATCCCTGTGATCGTCACTTGGTAAGTCACTG | |
| | | | ACTGTCTATGCCTGGGAAAGGGTGGGCAGGAGATGGGGC | |
| | | | AGTGCAGGAAAAGTGGCACTATGAACCCTGCAGCCCTAG | |
| | | | GAATGCATCTAGACAATTGTACTAACCTTCTTCTCTTTCCT | |
| | | | CTCCTGACAG | |
| 40396 | 60 | hRLP30 | CCCCGCAGCCATTCTAGCTAGCGGTACCAATAGCAACCG | 325 |
| | | | GCAGCTGCCCTCCGCTTTTGCTCCGCCCCTTCTGCTTGCG | |
| | | | ATCTGTTTCCGCTTCCGGTCCCGCAGTTCCGGCTCTGCCG | |
| | | | TGAAGAGCTTTGCATTGTGGGAAGTCTTTCCTTTCTCGTT | |
| | | | CCCCGGCCATCTTAGCGGCTGCTGTTGGTGAGTGGGCTCC | |
| | | | TACCGACCGAGGTTTAGGCAGCGCGGGGAGCTTTGCGGG | |
| | | | TTGCCATTTGTAACTCCGGATCCTAAAATTCCTGTCCTGTT | |
| | | | CTCTGTCTCTTCTAGGTTGGGGGCCGTCCCGCTCCTAAGG | |
| | | | CAGGAA | |
| 40397 | 61 | hRPS18 | AGCCCCGGAACCTTCGCTGTTCTCTTACCTATGAACCTTA | 243 |
| | | | CGAACTGTAAAGAAAGGCGCACCGGAAGTTGTGGTACCC | |
| | | | AAGCCATACTCTCATAAATCCAGCCAGGTCGCGCTGAAA | |
| | | | CAGTTTCCGGAAGCACTTCTCCTAGATCGCACCGCCTCTT | |
| | | | CCTCCTGGAAGCTATATAATGATATCGCGTCACTTCCGCT | |
| | | | CTCTCTTCCACAGGAGGCCTACACGCCGCCGCTTGTGCTG | |
| | | | CAGCC | |
| 40398 | 62 | CBA | CCACGTTCTGCTTCACTCTCCCCATCTCCCCCCCCTCCCC | 493 |
| | | | ACCCCCAATTTTGTATTTATTTATTTTTTAATTATTTTGTG | |
| | | | CAGCGATGGGGGCGGGGGGGGGGGGGGGGCGCGCGCCA | |
| | | | GGCGGGGCGGGGCGGGGCGAGGGGCGGGGCGGGGCGA | |
| | | | GGCGGAGAGGTGCGGCGGCAGCCAATCAGAGCGGCGCG | |
| | | | CTCCGAAAGTTTCCTTTTATGGCGAGGCGGCGGCGGCGG | |
| | | | CGGCCCTATAAAAAGCGAAGCGCGCGGCGGGCGGGAGC | |
| | | | GGGATCAGCCACCGCGGTGGCGGCCTAGAGTCGACGAG | |
| | | | GAACTGAAAAACCAGAAAGTTAACTGGTAAGTTTAGTCT | |
| | | | TTTTGTCTTTTATTTCAGGTCCCGGATCCGGTGGTGGTGC | |
| | | | AAATCAAAGAACTGCTCCTCAGTGGATGTTGCCTTTACT | |
| | | | TCTAGGCCTGTACGGAAGTGTTACTTCTGCTCTAAAAGC | |
| | | | TGCGGAATTGTACCCGCGGCCGATCCA | |
| 40399 | 63 | CBH | CGTTACATAACTTACGGTAAATGGCCCGCCTGGCTGACC | 565 |
| | | | GCCCAACGACCCCCGCCCATTGACGTCAATAGTAACGCC | |
| | | | AATAGGGACTTTCCATTGACGTCAATGGGTGGAGTATTT | |
| | | | ACGGTAAACTGCCCACTTGGCAGTACATCAAGTGTATCA | |
| | | | TATGCCAAGTACGCCCCCTATTGACGTCAATGACGGTAA | |
| | | | ATGGCCCGCCTGGCATTGTGCCCAGTACATGACCTTATG | |
| | | | GGACTTTCCTACTTGGCAGTACATCTACGTATTAGTCATC | |
| | | | GCTATTACCATGGTCGAGGTGAGCCCCACGTTCTGCTTC | |
| | | | ACTCTCCCCATCTCCCCCCCCTCCCCACCCCCAATTTTGT | |
| | | | ATTTATTTATTTTTTAATTATTTTGTGCAGCGATGGGGGC | |
| | | | GGGGGGGGGGGGGGGGCGCGCGCCAGGCGGGGCGGGG | |
| | | | CGGGGCGAGGGGGGGGCGGGGCGAGGCGGAGAGGTG | |
| | | | CGGCGGCAGCCAATCAGAGCGGCGCGCTCCGAAAGTTTC | |
| | | | CTTTTATGGCGAGGCGGCGGCGGCGGCGGCCCTATAAAA | |
| | | | AGCGAAGCGCGCGGCGGGCG | |
| 40400 | 64 | CMV core | GTGATGCGGTTTTGGCAGTACATCAATGGGCGTGGATAG | 204 |
| | | | CGGTTTGACTCACGGGGATTTCCAAGTCTCCACCCCATTG | |
| | | | ACGTCAATGGGAGTTTGTTTTGGCACCAAAATCAACGGG | |
| | | | ACTTTCCAAAATGTCGTAACAACTCCGCCCCATTGACGCA | |
| | | | AATGGGCGGTAGGCGTGTACGGTGGGAGGTCTATATAAG | |
| | | | CAGAGCT | |

### Method for Plasmid Nucleofection:

Immortalized neural progenitor cells were nucleofected as described in Example 1. Sequence-validated plasmids were diluted to concentrations of 200 ng/ul, 100 ng/ul, 50 ng/μL and 25 ng/μL, and 5 μL of each (1000 ng, 500 ng, 250 ng and 125 ng) were added to P3 solution containing 200,000 tdTomato mNPCs.

AAV viral production and QC, and AAV transduction and editing level assessment in mNPTC-tdT cells by FACS were conducted as described in Example 1.

### Results:

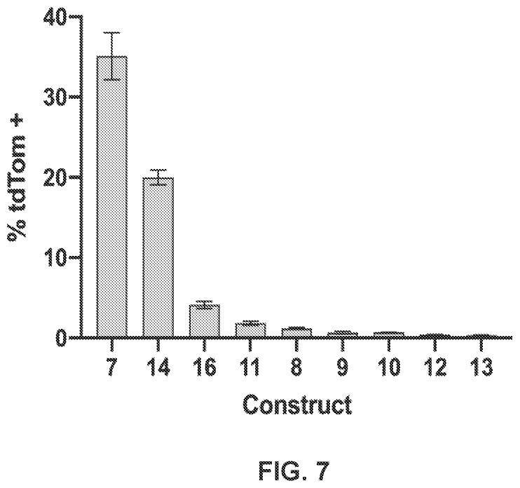

The results of FIG. 7 demonstrate that several different promoters with CasX protein 438, scaffold variant 174 and spacer targeting the tdTomato stop cassette (spacer 12.7, with sequence CTGCATTCTAGTTGTGGTTT (SEQ ID NO: 40800)), when delivered by nucleofection of AAV transgene plasmid, edit the target stop cassette in mNPCs at a dose of 1000 ng. These promoters ranged in length from over 700 nucleotides to as short as 81 nucleotides (Table 8). Among the promoters tested, construct 7 and 14 showed considerable editing potency.

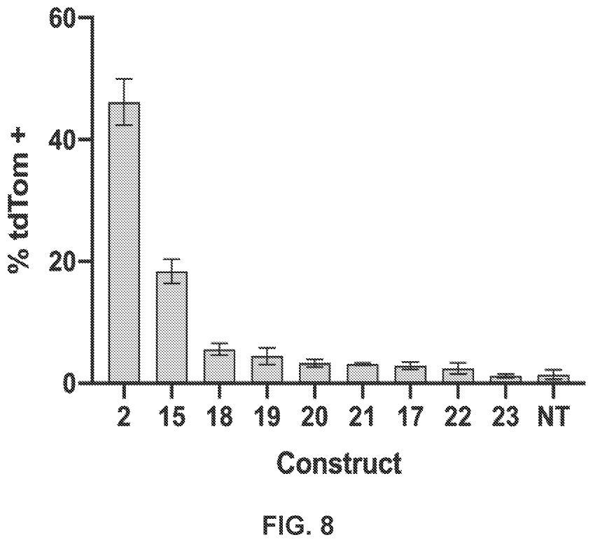

The results of FIG. 8 demonstrate that several short promoters combined with CasX variant 491, scaffold variant 174 and spacer 12.7, when delivered by nucleofection of AAV transgene plasmid, edit the target stop cassette in mNPCs at a dose of 500 ng. Other than construct 2, which had a promoter of 584 nucleotides, all of the constructs had promoters less than 250 nucleotides in length. Among the protein promoters tested, construct 15 showed considerable editing potency, especially given its short length (81 nucleotides).

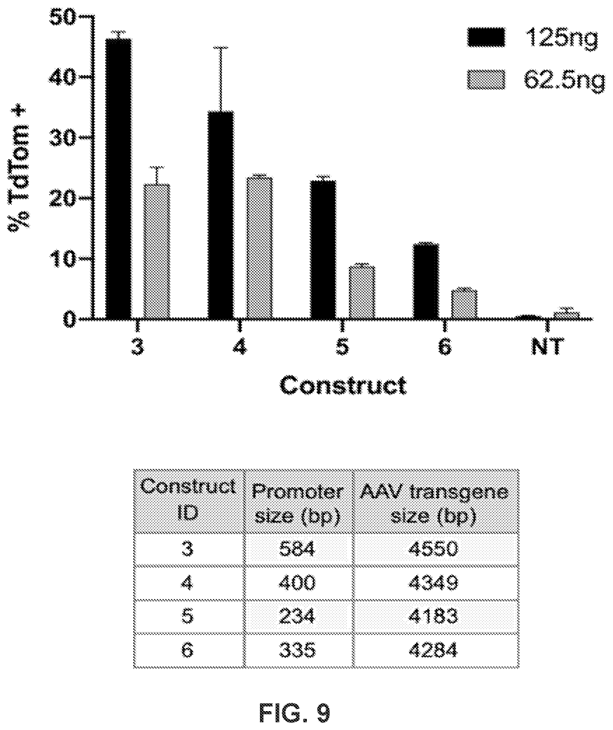

The results of FIG. 9 demonstrate that four lead promoters with CasX variant 491 and scaffold variant 174 with spacer 12.7, when delivered by nucleofection of AAV transgene plasmid, edit the target stop cassette in mNPCs at doses of 125 ng and 62.5 ng. Constructs 4, 5 and 6 have promoter lengths less than or equal to 400 nucleotides, and thus may maximize editing potency while minimizing AAV cargo capacity.

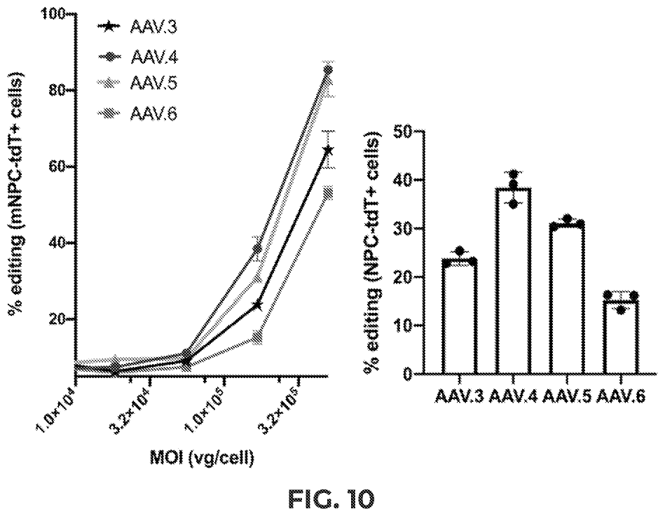

The results of FIG. 10 demonstrate that use of four promoter variants in the AAV also result in robust editing. Briefly, AAVs (AAV.3, AAV.4, AAV.5 and AAV.6) were generated with transgene constructs 3-6, respectively. Each construct showed dose-dependent editing at the target locus (FIG. 10, left panel). At an MOI of 2e5, AAV.4 showed editing at 38%±3% at the target locus, outperforming the other constructs (FIG. 10, right panel).

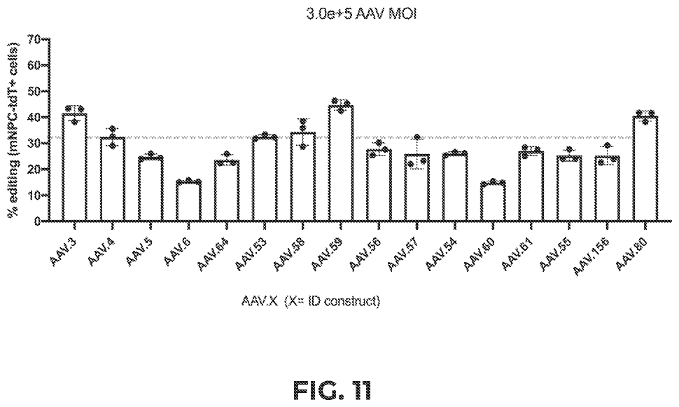

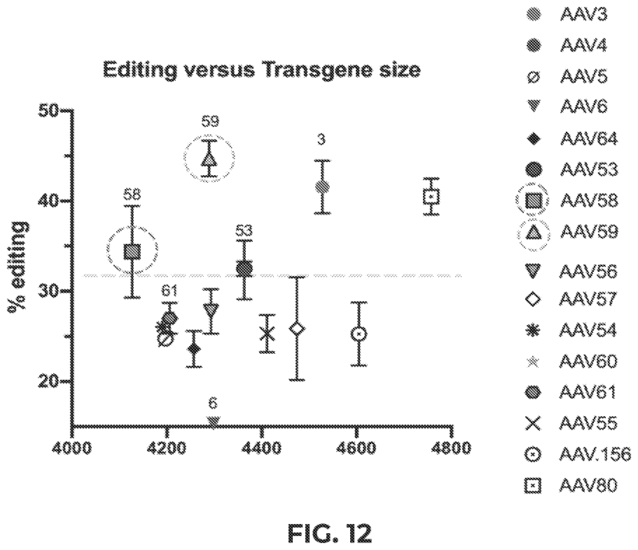

In the experiments portrayed in FIG. 11, several new protein promoters were compared against the top 4 protein promoter variants identified previously (AAV.3, AAV.4, AAV.5 and AAV.6). Briefly, AAVs were generated with corresponding transgene constructs and transduced in tdTomato mNPCs. At an MOI of 3e5 5 days after transduction, multiple promoters displayed improved editing (FIG. 11). In particular, constructs 58 and 59 had editing activity above 30% while minimizing transgene size (FIG. 12). Construct 58 and 59 contained promoters that are 420 and 258 bp smaller, respectively, than construct 3, yet resulted in similar or improved editing of the target locus. In particular, inclusion of an intron in the promoter of construct 59 led to increased editing compared to construct 58, lacking the intron, suggesting that the inclusion of introns in the AAV construct promoters is beneficial.

The results demonstrate that expression of small CRISPR proteins (such as CasX) can be enhanced by utilizing long promoters that would otherwise be unusable with traditional CRISPR proteins due to the size constraints of the AAV genome. Furthermore, combining short promoters with small CRISPR proteins (such as CasX) allows for significant reductions in AAV transgene cargo capacity without compromising expression efficiency. This conservation of space allows for the inclusion of additional accessory elements, such as enhancers and regulatory elements in the transgene, which would enable increased editing potential.

### Example 5: Small CRISPR Systems Potency is Enhanced by AAV Vector RNA Promoter Choice

Experiments were conducted to demonstrate that the editing potency of small CRISPR systems (such as CasX) can be enhanced if certain promoters are chosen for expression of the guide RNA, which recognizes target DNA for editing, in an AAV vector. By using RNA promoters with different strengths, guide RNA expression can be modulated, which affects editing potency. The AAV platform based on the CasX system provides enough cargo space in the AAV to include at least 2 independent promoters for the expression of two guide RNAs. By combining promoters with different levels of expression, expression of multiple guide RNAs can be tuned within a single AAV transgene. Engineering shorter versions of RNA promoters that result in retained editing potency also enables increased engineering space for the addition of other accessory elements in the AAV transgene.

### Materials and Methods:

The methods of Example 1 were used for cloning and quality control of the constructs, as well as for plasmid nucleofection and AAV production, transduction, and FACS analyses. The sequences of the Pol III promoters are presented in Table 9. The sequences of the additional components of the AAV constructs, with the exception of sequences encoding the CasX (Table 21) and the one or more gRNA (Tables 18 and 19), are listed in Table 26.

TABLE 9
Construct RNA promoter sequences

| SEQ | | | | |
|---|---|---|---|---|
| ID | Construct | Pol III | | Promoter |
| NO: | ID | promoter | Sequences of engineered promoters | size (bp) |
| 40401 | 3, 53, | human U6 | GAGGGCCTATTTCCCATGATTCCTTCATATTTGCATA | 241 |
| | 157 | | TACGATACAAGGCTGTTAGAGAGATAATTGGAATTA | |
| | | | ATTTGACTGTAAACACAAAGATATTAGTACAAAATA | |
| | | | CGTGACGTAGAAAGTAATAATTTCTTGGGTAGTTTGC | |
| | | | AGTTTTAAAATTATGTTTTAAAATGGACTATCATATG | |
| | | | CTTACCGTAACTTGAAAGTATTTCGATTTCTTGGCTTT | |
| | | | ATATATCTTGTGGAAAGGAC | |
| 40402 | 32, 158 | H1 | GAACGCTGACGTCATCAACCCGCTCCAAGGAATCGC | 215 |
| | | | GGGCCCAGTGTCACTAGGCGGGAACACCCAGCGCGC | |
| | | | GTGCGCCCTGGCAGGAAGATGGCTGTGAGGGACAGG | |
| | | | GGAGTGGCGCCCTGCAATATTTGCATGTCGCTATGTG | |
| | | | TTCTGGGAAATCACCATAAACGTGAAATGTCTTTGGA | |
| | | | TTTGGGAATCTTATAAGTTCTGTATGAGACCAC | |
| 40403 | 33 | 7SK | CTGCAGTATTTAGCATGCCCCACCCATCTGCAAGGCA | 244 |
| | | | TTCTGGATAGTGTCAAAACAGCCGGAAATCAAGTCC | |
| | | | GTTTATCTCAAACTTTAGCATTTTGGGAATAAATGAT | |
| | | | ATTTGCTATGCTGGTTAAATTAGATTTTAGTTAAATTT | |
| | | | CCTGCTGAAGCTCTAGTACGATAAGTAACTTGACCTA | |
| | | | AGTGTAAAGTTGAGATTTCCTTCAGGTTTATATAGCT | |
| | | | TGTGCGCCGCCTGGGTACCTCG | |
| 40404 | 85/89 | hU6 | GAGGGCCTATTTCCCATGATTCCTTCATATTTGCATA | 103 |
| | | variant 1 | TACGATAGCTTACCGTAACTTGAAAGTATTTCGATTT | |
| | | | CTTGGCTTTATATATCTTGTGGAAAGGAC | |
| 40405 | 86 | hU6 | TTTCGATTTCTTGGCTTTATATATCTTGTGGAAAGGA | 38 |
| | | variant 2 | C | |
| 40406 | 87 | hU6 | ATACGATAGCTTACCGTAACTTGAAAGTATTTCGATT | 67 |
| | | variant 3 | TCTTGGCTTTATATATCTTGTGGAAAGGAC | |
| 40407 | 88 | hU6 | GAGGGCCTATTTCCCATGATTCCTTCATATTTGCATTT | 79 |
| | | variant 4 | TCGATTTCTTGGCTTTATATATCTTGTGGAAAGGACG | |
| | | | AAAC | |
| 40408 | 90 | hU6 | GAGGGCCTATTTCCCATGATTCCTTCATATTTGCATA | 111 |
| | | variant 5 | TTTGCATATACGATAGCTTACCGTAACTTGAAAGTAT | |
| | | | TTCGATTTCTTGGCTTTATATATCTTGTGGAAAGGAC | |
| 40409 | 91 | hU6 | GAGGGCCTATTTCCCATGATTCCTTCATATTTGCATA | 127 |
| | | variant 6 | TTTGCATATTTGCATATTTGCATATACGATAGCTTAC | |
| | | | CGTAACTTGAAAGTATTTCGATTTCTTGGCTTTATAT | |
| | | | ATCTTGTGGAAAGGAC | |
| 40410 | 92 | hU6 | GAGGGCCTATTTCCCATGATTCCTTCATATTTCCCAT | 123 |
| | | variant 7 | GATTCCTTCATATTTGCATATACGATAGCTTACCGTA | |
| | | | ACTTGAAAGTATTTCGATTTCTTGGCTTTATATATCTT | |
| | | | GTGGAAAGGAC | |
| 40411 | 93 | hU6 | GAGGGCCTATTTCCCATGATTCCTTCATATTTCCCAT | 143 |
| | | variant 8 | GATTCCTTCATATTTCCCATGATTCCTTCATATTTGCA | |
| | | | TATACGATAGCTTACCGTAACTTGAAAGTATTTCGAT | |
| | | | TTCTTGGCTTTATATATCTTGTGGAAAGGAC | |
| 40412 | 94 | hU6 | GAGGGCCTATTTCCCATGATTCCTTCATATTTGCATA | 131 |
| | | variant 9 | TTTCCCATGATTCCTTCATATTTGCATATACGATAGCT | |
| | | | TACCGTAACTTGAAAGTATTTCGATTTCTTGGCTTTAT | |
| | | | ATATCTTGTGGAAAGGAC | |
| 40413 | 95 | hU6 | GAGGGCCTATTTCCCATGATTCCTTCATATTTGCATA | 159 |
| | | variant 10 | TTTCCCATGATTCCTTCATATTTGCATATTTCCCATGA | |
| | | | TTCCTTCATATTTGCATATACGATAGCTTACCGTAAC | |
| | | | TTGAAAGTATTTCGATTTCTTGGCTTTATATATCTTGT | |
| | | | GGAAAGGAC | |
| 40414 | 96 | hU6 | GAGGGCCTATTTCCCATGATTCCTTCATATGCAAATA | 103 |
| | | variant 11 | TACGATAGCTTACCGTAACTTGAAAGTATTTCGATTT | |
| | | | CTTGGCTTTATATATCTTGTGGAAAGGAC | |
| 40415 | 97 | hU6 | GAGGGCCTATTTCCCATGATTCCTTCATATGCAAATA | 111 |
| | | variant 12 | TGCAAATATACGATAGCTTACCGTAACTTGAAAGTAT | |
| | | | TTCGATTTCTTGGCTTTATATATCTTGTGGAAAGGAC | |
| 40416 | 98 | hU6 | GAGGGCCTATTTCCCATGATTCCTTCATATGCAAATA | 127 |
| | | variant 13 | TGCAAATATGCAAATATGCAAATATACGATAGCTTA | |
| | | | CCGTAACTTGAAAGTATTTCGATTTCTTGGCTTTATAT | |
| | | | ATCTTGTGGAAAGGAC | |
| 40417 | 99 | hU6 | GAGGGCCTATGCAAATATGAAGGAATCATGGGAAAT | 103 |
| | | variant 14 | ATACGATAGCTTACCGTAACTTGAAAGTATTTCGATT | |
| | | | TCTTGGCTTTATATATCTTGTGGAAAGGAC | |
| 40418 | 100 | hU6 | GAGGGCCTATGCAAATATGAAGGAATCATGGGAAAT | 131 |
| | | variant 15 | ATGCAAATATGAAGGAATCATGGGAAATATACGATA | |
| | | | GCTTACCGTAACTTGAAAGTATTTCGATTTCTTGGCT | |
| | | | TTATATATCTTGTGGAAAGGAC | |
| 40419 | 101 | hU6 | GAGGGCCTATGCAAATATGAAGGAATCATGGGAAAT | 159 |
| | | variant 16 | ATGCAAATATGAAGGAATCATGGGAAATATGCAAAT | |
| | | | ATGAAGGAATCATGGGAAATATACGATAGCTTACCG | |
| | | | TAACTTGAAAGTATTTCGATTTCTTGGCTTTATATATC | |
| | | | TTGTGGAAAGGAC | |
| 40420 | 102 | hU6 | GAGGGCCTATTTCCCATGATTCCTTCATATTTGCATA | 128 |
| | | variant 17 | TACGTTTGACTGTAAATACGTGACGTAGAATAGCTTA | |
| | | | CCGTAACTTGAAAGTATTTCGATTTCTTGGCTTTATAT | |
| | | | ATCTTGTGGAAAGGAC | |
| 41010 | 159 | H1 core | ATATTTGCATGTCGCTATGTGTTCTGGGAAATCACCATAAAC | 91 |
| | | | GTGAAATGTCTTTGGATTTGGGAATCTTATAAGTTCTGTATG | |
| | | | AGACCAC | |
| 41011 | 160 | H1 core + | ATATTTAGCATGTCGCTATGTGTTCTGGGAAATCACCATAAA | 92 |
| | | 7SK | CGTGAAATGTCTTTGGATTTGGGAATCTTATAAGTTCTGTAT | |
| | | hybrid 1 | GAGACCAC | |
| 41012 | 161 | H1 core + | ATATTTGCATGTCTGCAAGGCATTCTGGATAGTCACCATAAA | 92 |
| | | 7SK | CGTGAAATGTCTTTGGATTTGGGAATCTTATAAGTTCTGTAT | |
| | | hybrid 2 | GAGACCAC | |
| 41013 | 162 | H1 core + | ATATTTGCATGTCGCTATGTGTTCTGGGAAATTGACCTAAGT | 91 |
| | | 7SK | GTAAAGTGTCTTTGGATTTGGGAATCTTATAAGTTCTGTATG | |
| | | hybrid 3 | AGACCAC | |
| 41014 | 163 | H1 core + | ATATTTAGCATTCTGCAAGGCATTCTGGATAGTGACCTAAGT | 91 |
| | | 7SK | GTAAAGTGTCTTTGGATTTGGGAATCTTATATATTCTGTATG | |
| | | hybrid 4 | AGACCAC | |
| 41015 | 164 | H1 core + | ATATTTAGCATTCTGCAAGGCATTCTGGATAGTCACCATAAA | 92 |
| | | 7SK | CGTGAAATGTCTTTGGATTTGGGAATCTTATAAGTTCTGTAT | |
| | | hybrid 5 | GAGACCAC | |
| 41016 | 165 | H1 core + | ATATTTGCATGTCGCTATGTGTTCTGGGAAACTTGACCTAAG | 91 |
| | | 7SK | TGTAAAGTTGAGATTTCCTTCAGGTTTTATAAGTTCTGTATG | |
| | | hybrid 6 | AGACCAC | |
| 41017 | 166 | H1 core + | ATATTTGCATGTCGCTATGTGTTCTGGGAAATCACCATAAAC | 91 |
| | | 7SK | GTGAAATTTGAGATTTCCTTCAGGTTTTATAAGTTCTGTATG | |
| | | hybrid 7 | AGACCAC | |
| 41018 | 167 | H1 core + | ATATTTGCATGTCGCTATGTGTTCTGGGAAACTTGACCTAAG | 91 |
| | | 7SK | TGTAAAGTTGAGATTTCCTTCAGGTTTATATAGTTCTGTATG | |
| | | hybrid 8 | AGACCAC | |
| 41019 | 168 | H1 core + | ATATTTAGCATGTCGCTATGTGTTCTGGGAAACTTGACCTAA | 92 |
| | | 7SK | GTGTAAAGTTGAGATTTCCTTCAGGTTTATATAGTTCTGTAT | |
| | | hybrid 9 | GAGACCAC | |
| 41020 | 169 | H1 core + | ATATTTGCATGATTTCCCATGATTCCTTCATTCACCATAAAC | 91 |
| | | U6 | GTGAAATGTCTTTGGATTTGGGAATCTTATAAGTTCTGTATG | |
| | | hybrid 1 | AGACCAC | |
| 41021 | 170 | H1 core + | ATATTTGCATGTCGCTATGTGTTCTGGGAAATCTTACCGTAA | 94 |
| | | U6 | CTTGAAAGTAGTCTTTGGATTTGGGAATCTTATAAGTTCTGT | |
| | | hybrid 2 | ATGAGACCAC | |
| 41022 | 171 | H1 core + | ATATTTGCATATTTCCCATGATTCCTTCATCTTACCGTAACT | 92 |
| | | 7SK + U6 | TGAAAGTAGTCTTTGGATTTGGGAATCTTATATATTCTGTAT | |
| | | hybrid 1 | GAGACCAC | |
| 41023 | 172 | H1 core + | ATATTTGCATATTTCCCATGATTCCTTCATTCACCATAAACG | 90 |
| | | U6 | TGAAATGTCTTTGGATTTGGGAATCTTATAAGTTCTGTATGA | |
| | | hybrid 3 | GACCAC | |
| 41024 | 173 | H1 core + | ATATTTGCATGTCGCTATGTGTTCTGGGAAACTCTTACCGTA | 94 |
| | | 7SK + U6 | ACTTGAAAGTATGAGATTTCCTTCAGGTTTTATAAGTTCTGT | |
| | | hybrid 2 | ATGAGACCAC | |
| 41025 | 174 | H1 core + | ATATTTGCATGTCGCTATGTGTTCTGGGAAACTCTTACCGTA | 94 |
| | | 7SK + U6 | ACTTGAAAGTATGAGATTTCCTTCAGGTTTATATAGTTCTGT | |
| | | hybrid 3 | ATGAGACCAC | |
| 41026 | | hU6 | GCCACCTCTTTTGCATATTGGCACCCACAATCCACCGCGGCT | 247 |
| | | isoform 2 | ATGAGGCCAGTATAAGGCGGTAAAATTACGATAAGATATGGG | |
| | | | ATTTTACGTGATCGAAGACATCAAAGTAAGCGTAAGCACGAA | |
| | | | AGTTGTTCTGCAACATACCACTGTAGGAAATTATGCTAAATA | |
| | | | TGAAACCGACCATAAGTTATCCTAACCAAAAGATGATTTGAT | |
| | | | TGAAGGGCTTAAAATAGGTGTGACAGTAACCCTTGAGTC | |
| 41027 | | hU6 | TCCCTTACCCAGGGTGCCCCGGGCGCTCATTTGCATGTCCCA | 249 |
| | | isoform 3 | CCCAACAGGTAAACCTGACAGATCGGTCGCGGCCAGGTACGG | |
| | | | CCTGGCGGTCAGAGCACCAAACTTACGAGCCTTGTGATGAGT | |
| | | | TCCGTTACATGAAATTCTCCTAAAGGCTCCAAGATGGACAGG | |
| | | | AAAGCGCTCGATTCGGTTACCGTAAGGAAAACAAATGAGAAA | |
| | | | CTCCCGTGCCTTATAAGACCTGGGGACGGACTTATTTGC | |
| 41028 | | hU6 | CCCTTACCCAGGGTGCCCCGGGCGCTCATTTGCATGTCCCAC | 249 |
| | | isoform 4 | CCAACAGGTAAACCTGACAGGTCATCGCGGCCAGGTACGACC | |
| | | | TGGCGGTCAGAGCACCAAACATACGAGCCTTGTGATGAGTTC | |
| | | | CGTTGCATGAAATTCTCCCAAAGGCTCCAAGATGGACAGGAA | |
| | | | AGGGCGCGGTTCGGTCACCGTAAGTAGAATAGGTGAAAGACT | |
| | | | CCCGTGCCTTATAAGGCCTGTGGGTGACTTCTTCTCAAC | |
| 41029 | | hU6 | CAGGCTCTGCCCCGCCTCCGGGGCTATTTGCATACGACCATT | 249 |
| | | isoform 5 | TCCAGTAATTCCCAGCAGCCACCGTAGCTATATTTGGTAGAA | |
| | | | CAACGAGCACTTTCTCAACTCCAGTCAATAACTACGTTAGTT | |
| | | | GCATTACACATTGGGCTAATATAAATAGAGGTTAAATCTCTA | |
| | | | GGTCATTTAAGAGAAGTCGGCCTATGTGTACAGACATTTGTT | |
| | | | CCAGGGGCTTTAAATAGCTGGTGGTGGAACTCAATATTC | |

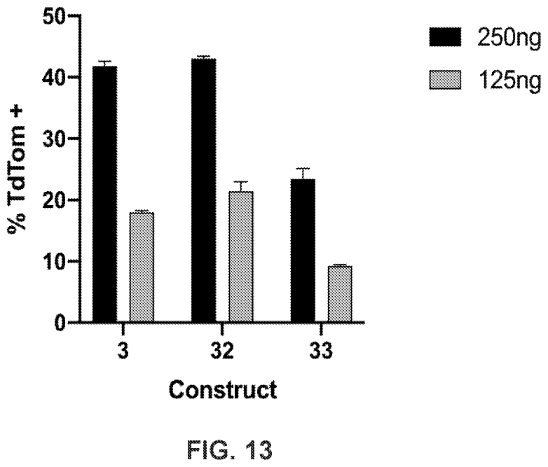

The results portrayed in FIG. 13 demonstrate that three distinct RNA promoters with protein 491, scaffold variant 174 and spacer 12.7, when delivered by nucleofection of AAV transgene plasmid, edit the target stop cassette in mNPCs at doses of 250 ng and 125 ng. Constructs 3 and 32 have similar activity, editing at the target locus with 42% efficiency. Construct 33 shows ˜56% of the activity of constructs 3 and 32.

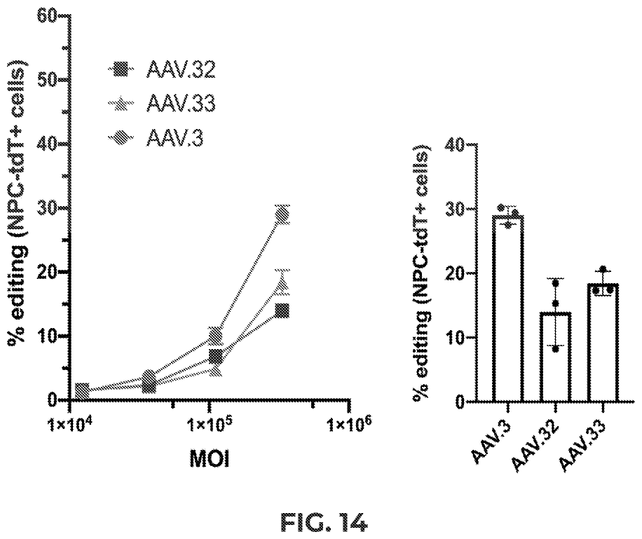

The results portrayed in FIG. 14 demonstrate that the same three distinct promoters with protein 491, scaffold variant 174 and spacer 12.7 when delivered as AAV edit the target stop cassette in mNPCs. AAV.3, AAV.32, AAV.33 were generated with transgene constructs 3, 32 and 33 respectively. Each vector displayed dose-dependent editing at the target locus (FIG. 14, left panel). At an MOI of 3e5, AAV.32 and AAV.33 had 50-60% of the potency of AAV.3 (FIG. 14, right panel).

The results of FIG. 15 demonstrate that constructs having one of four different truncations of the U6 promoter with protein 491, scaffold variant 174 and spacer 12.7, when delivered by nucleofection of AAV transgene plasmid, were each able to edit the target stop cassette at differential levels in mNPCs at doses of 250 ng and 125 ng. Construct 85 had 33% of the potency of the base construct 53 while constructs 86, 87 and 88 didn't show any editing with, and were comparable to, a non-targeting control.

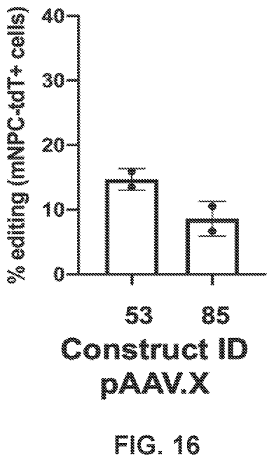

FIG. 16 presents results of an experiment comparing editing in mNPCs between base construct 53 to construct 85, when delivered as AAV. AAV.85 was able to edit at 7% compared to 15% for AAV.53 at an MOI of 3e5, consistent with the results from FIG. 15.

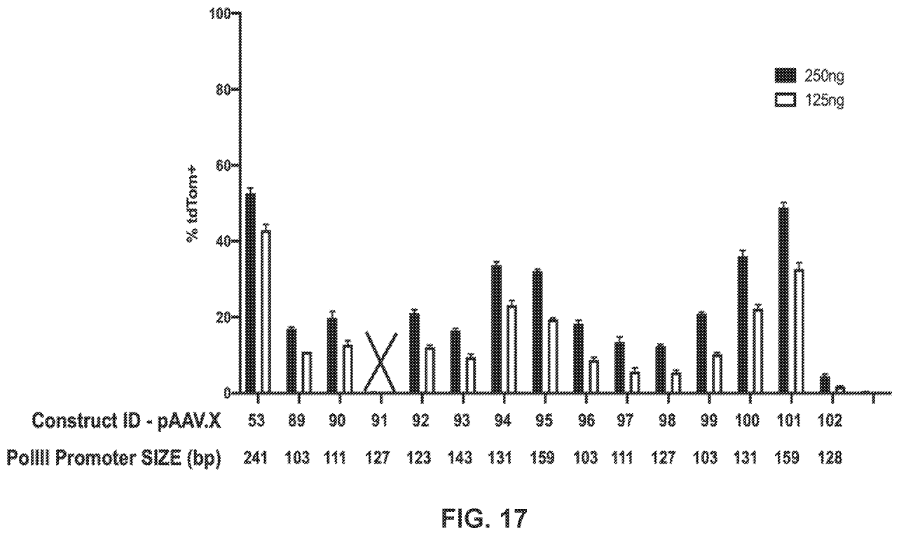

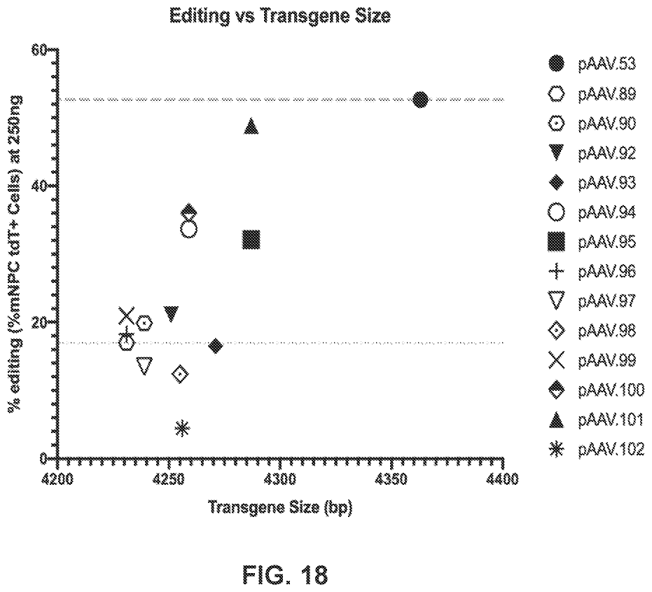

The results of FIG. 17 demonstrate that that constructs with engineered U6 promoters designed to minimize the size of the promoter relative to the base U6 of construct 53, with encoded CasX protein 491, scaffold variant 174 and spacer 12.7, when delivered by nucleofection of AAV transgene plasmid, were able to edit the target stop cassette at differential levels in mNPCs at doses of 250 ng and 125 ng. One cluster of constructs (89, 90, 92, 93, 96, 97, 98, and 99) all edited in the range of 15-20%, compared to 55% for construct 53. Other Pol II variants (construct 94, 95 and 100) all exhibited higher levels of editing at around 32% editing while construct 101 resulted in 48% editing. These promoters are all smaller than the Pol III promoter in the base construct 53, as shown in the scatterplot of FIG. 18, depicting transgene size of all AAV variants tested having engineered U6 RNA promoters on the X-axis vs. percent of mNPCs edited on the Y-axis.

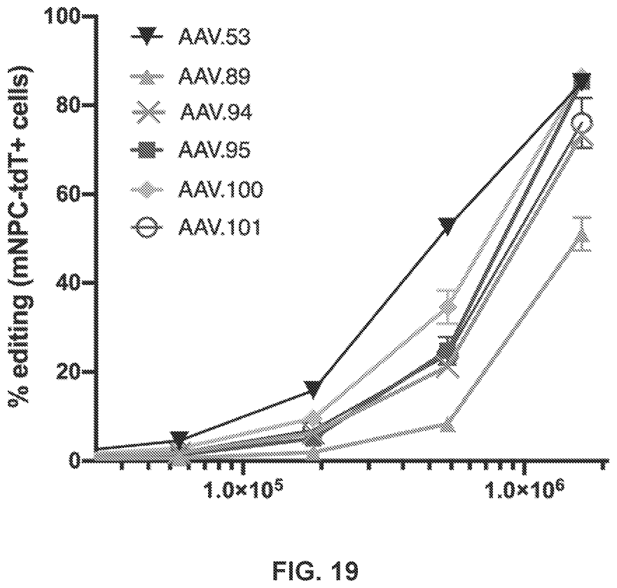

The results of FIG. 19 show that constructs with engineered U6 promoters with protein 491, scaffold variant 174 and spacer 12.7, when delivered as AAV, were able to edit the target stop cassette in mNPCs in a dose-dependent fashion. Variable rates of editing with AAV with constructs AAV.94, AAV.95, AAV.100, and AAV.101 were seen, all editing at rates between the base construct AAV.53 and AAV.89, which has the same Pol III promoter as AAV.85 from FIGS. 15 and 16.

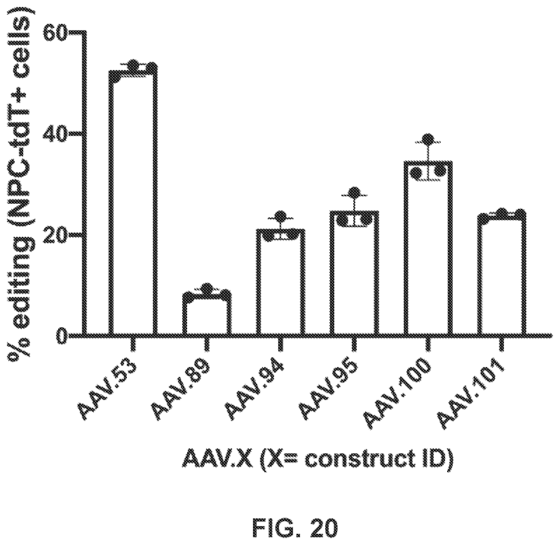

The results of FIG. 20 show that constructs with engineered U6 promoters with CasX protein 491, scaffold variant 174 and spacer 12.7, when delivered as AAV, were able to edit the target stop cassette in mNPCs. Variable rates of editing with AAV with constructs AAV.94, AAV.95, AAV.100, and AAV.101 were seen, all editing at rates between the base construct AAV.53 and AAV.89, which has the same Pol III promoter as AAV.85 from FIGS. 15 and 16. FIG. 21 shows the results as a scatterplot of editing versus transgene size.

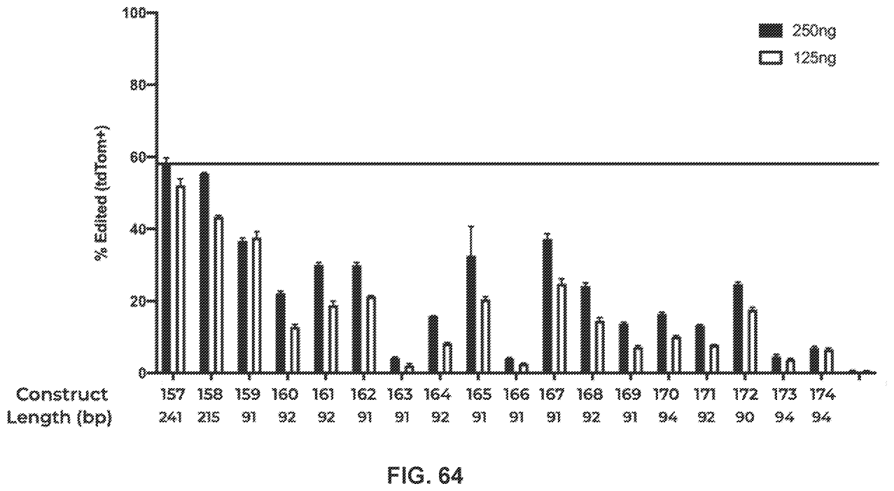

The results depicted in FIG. 64 demonstrate that constructs of rationally engineered Pol III promoters, with sequences encoding for CasX protein 491, scaffold variant 174, and spacer 12.7, were able to edit the target tdTomato stop cassette at varying efficiencies when nucleofected as AAV transgene plasmids into mouse NPCs at doses 250 ng and 125 ng. Constructs 159 to 174 were designed to minimize the size of the promoter relative to the base U6 (construct ID 157) or H1 (construct ID 158) promoter, and constructs 160 to 174 were engineered as short, hybrid variants based on a core region of the H1 promoter (construct 159) with variations of domain swaps from 7SK and/or U6 promoters. FIG. 64 shows that most of these promoter variants, which are substantially shorter than the base U6 and H1 promoters, were able to function as Pol III promoters to drive sufficient gRNA transcription and editing at the tdTomato locus. Specifically, constructs 159, 161, 162, 165, and 167 were able to achieve at least 30% editing at the higher dose of 250 ng. These variants serve as promoter alternatives in AAV construct design that would permit significant reductions in AAV cargo capacity while driving adequate gRNA expression for targeted editing.

The results of the experiments demonstrate that expression of small CRISPR system (such as CasX and guides) can be modulated in a selective way by utilizing alternative RNA promoters. While most other CRISPR systems do not have sufficient space to include a separate promoter to express the guide RNA, the CRISPR system described herein enables the use of several possible gRNA promoters of varying lengths in the transgene to differentially control expression and editing. The data also support that shorter versions of Pol III promoters can be engineered that retain the ability to facilitate transcription of functional guides. This quality is an important feature of the AAV system described herein in order to save transgene space for additional engineering or inclusion of additional promoters and/or accessory elements. Furthermore, adjusting other elements in our system allows for the combination of multiple gRNA promoters, including ones with varying potencies.

### Example 6: Small CRISPR Protein Potency is Enhanced by the Choice of Poly(A) in AAV Vectors

Experiments were conducted to demonstrate that small CRISPR proteins (such as CasX) can be expressed in an AAV genome utilizing a variety of polyadenylation (poly(A)) signals. Specifically, smaller CRISPR systems enable the inclusion of larger poly(A) signals. In addition, experiments were conducted to demonstrate that the inclusion of shorter synthetic poly(A) signals in the constructs allows for further reductions in AAV transgene cargo capacity.

### Materials and Methods:

### Cloning and QC:

Poly(A) signals within the AAV genome were separated by restriction enzyme sites to allow for modular cloning. Parts were ordered as gene fragments from Twist, PCR amplified, digested with corresponding restriction enzymes, cleaned, then ligated into a vector also digested with the same enzymes.

The methods of Example 1 were used for cloning and quality control of the constructs, as well as for plasmid nucleofection and FACS analyses. The sequences of the poly(A) sequences are presented in Table 10. The sequences of the additional components of the AAV constructs, with the exception of sequences encoding the CasX (Table 21) and the one or more gRNA (Tables 18 and 19), are listed in Table 26.

TABLE 10
Poly(A) sequences

| SEQ | | | | |
|---|---|---|---|---|
| ID | Construct | | | Size |
| NO: | ID | Poly(A) | Sequence | (bp) |
| 40421 | 1, 3, 37 | bGH | CTGTGCCTTCTAGTTGCCAGCCATCTGTTGTTTGCCCCTCCCC | 208 |
| | | | CGTGCCTTCCTTGACCCTGGAAGGTGCCACTCCCACTGTCCTT | |
| | | | TCCTAATAAAATGAGGAAATTGCATCGCATTGTCTGAGTAGG | |
| | | | TGTCATTCTATTCTGGGGGGTGGGGTGGGGCAGGACAGCAAG | |
| | | | GGGGAGGATTGGGAAGAGAATAGCAGGCATGCTGGGGA | |
| 40422 | 24 | hGH | GGGTGGCATCCCTGTGACCCCTCCCCAGTGCCTCTCCTGGCCC | 623 |
| | | | TGGAAGTTGCCACTCCAGTGCCCACCAGCCTTGTCCTAATAA | |
| | | | AATTAAGTTGCATCATTTTGTCTGACTAGGTGTCCTTCTATAA | |
| | | | TATTATGGGGTGGAGGGGGGTGGTATGGAGCAAGGGGCAAG | |
| | | | TTGGGAAGACAACCTGTAGGGCCTGCGGGGTCTATTGGGAAC | |
| | | | CAAGCTGGAGTGCAGTGGCACAATCTTGGCTCACTGCAATCT | |
| | | | CCGCCTCCTGGGTTCAAGCGATTCTCCTGCCTCAGCCTCCCGA | |
| | | | GTTGTTGGGATTCCAGGCATGCATGACCAGGCTCAGCTAATT | |
| | | | TTTGTTTTTTTGGTAGAGACGGGGTTTCACCATATTGGCCAGG | |
| | | | CTGGTCTCCAACTCCTAATCTCAGGTGATCTACCCACCTTGGC | |
| | | | CTCCCAAATTGCTGGGATTACAGGCGTGAACCACTGCTCCCTT | |
| | | | CCCTGTCCTTCTGATTTTAAAATAACTATACCAGCAGGAGGA | |
| | | | CGTCCAGACACAGCATAGGCTACCTGGCCATGCCCAACCGGT | |
| | | | GGGACATTTGAGTTGCTTGCTTGGCACTGTCCTCTCATGCGTT | |
| | | | GGGTCCACTCAGTAGATGCCTGTTGAATT | |
| 40423 | 25 | hGH | GGGTGGCATCCCTGTGACCCCTCCCCAGTGCCTCTCCTGGCCC | 477 |
| | | short | TGGAAGTTGCCACTCCAGTGCCCACCAGCCTTGTCCTAATAA | |
| | | | AATTAAGTTGCATCATTTTGTCTGACTAGGTGTCCTTCTATAA | |
| | | | TATTATGGGGTGGAGGGGGGTGGTATGGAGCAAGGGGCAAG | |
| | | | TTGGGAAGACAACCTGTAGGGCCTGCGGGGTCTATTGGGAAC | |
| | | | CAAGCTGGAGTGCAGTGGCACAATCTTGGCTCACTGCAATCT | |
| | | | CCGCCTCCTGGGTTCAAGCGATTCTCCTGCCTCAGCCTCCCGA | |
| | | | GTTGTTGGGATTCCAGGCATGCATGACCAGGCTCAGCTAATT | |
| | | | TTTGTTTTTTTGGTAGAGACGGGGTTTCACCATATTGGCCAGG | |
| | | | CTGGTCTCCAACTCCTAATCTCAGGTGATCTACCCACCTTGGC | |
| | | | CTCCCAAATTGCTGGGATTACAGGCGTGAACCACTGCTCCCTT | |
| | | | CCCTGTCCTT | |
| 40424 | 26 | HSVTK | CGGCAATAAAAAGACAGAATAAAACGCACGGGTGTTGGGTC | 49 |
| | | | GTTTGTTC | |
| 40425 | 27 | SynPolyA | AATAAAAGATCTTTATTTTCATTAGATCTGTGTGTTGGTTTTTT | 49 |
| | | | GTGTG | |
| 40426 | 28 | SV40 | AACTTGTTTATTGCAGCTTATAATGGTTACAAATAAAGCAAT | 122 |
| | | | AGCATCACAAATTTCACAAATAAAGCATTTTTTTCACTGCATT | |
| | | | CTAGTTGTGGTTTGTCCAAACTCATCAATGTATCTTA | |
| 40427 | 29 | SV40 | AACTTGTTTATTGCAGCTTATAATGGTTACAAATAAAGCAAT | 82 |
| | | short | AGCATCACAAATTTCACAAATAAAGCATTTTTTTCACTGC | |
| 40428 | 30 | bglob | GCTCGCTTTCTTGCTGTCCAATTTCTATTAAAGGTTCCTTTGTT | 395 |
| | | | CCCTAAGTCCAACTACTAAACTGGGGGATATTATGAAGGGCC | |
| | | | TTGAGCATCTGGATTCTGCCTAATAAAAAACATTTATTTTCAT | |
| | | | TGCAATGATGTATTTAAATTATTTCTGAATATTTTACTAAAAA | |
| | | | GGGAATGTGGGAGGTCAGTGCATTTAAAACATAAAGAAATG | |
| | | | AAGAGCTAGTTCAAACCTTGGGAAAATACACTATATCTTAAA | |
| | | | CTCCATGAAAGAAGGTGAGGCTGCAAACAGCTAATGCACATT | |
| | | | GGCAACAGCCCCTGATGCCTATGCCTTATTCATCCCTCAGAA | |
| | | | AAGGATTCAAGTAGAGGCTTGATTTGGAGGTTAAAGTTTTGC | |
| | | | TATGCTGTATTTTA | |
| 40429 | 31 | bglobsh | AATAAAGGAAATTTATTTTCATTGCAATAGTGTGTTGGAATTT | 56 |
| | | ort | TTTGTGTCTCTCA | |
| 40430 | 34 | SV40po | TATTTGTGAAATTTGTGATGCTATTGCTTTATTTGTAACCATCT | 181 |
| | | lyA late | AGCTTTATTTGTGAAATTTGTGATGCTATTGCTTTATTTGTAA | |
| | | | CCATTATAAGCTGCAATAAACAAGTTAACAACAACAATTGCA | |
| | | | TTCATTTTATGTTTCAGGTTCAGGGGGAGATGTGGGAGGTTTT | |
| | | | TTAAAGCGG | |

Methods for plasmid nucleofection and assessing activity by FACS were conducted as described in Example 1.

### Results:

The results portrayed in FIG. 22 demonstrate that constructs with several alternative poly(A) signals with CasX variant 491, scaffold variant 174 and spacer 12.7, when delivered by nucleofection of AAV transgene plasmid, were able to edit the target stop cassette in mNPCs at doses of 250 ng and 125 ng. Construct 3 showed the highest potency out of the three constructs tested in this experiment, editing the target locus at 60% efficiency (250 ng dose). Constructs 28 and 29, which have poly(A) sequences that are 59% and 39% of the size of the poly(A) sequence of construct 3, respectively (see Table 11), edited at 21% and 24% respectively (250 ng dose).

TABLE 11
Poly(A) construct variants

| Construct ID | Poly(A) Signal Size (bp) | AAV Transgene Size (bp) |
|---|---|---|
| 3 | 208 | 4550 |
| 25 | 477 | 4795 |
| 26 | 49 | 4367 |
| 27 | 49 | 4367 |
| 28 | 122 | 4440 |
| 29 | 82 | 4400 |
| 30 | 395 | 4713 |
| 31 | 56 | 4374 |
| 34 | 186 | 4565 |
| 37 | 208 | 4619 |

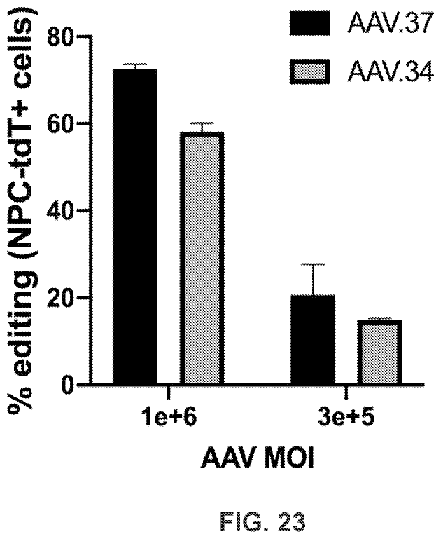

The results portrayed in FIG. 23 demonstrate that the two different poly(A) signals with protein 491, scaffold variant 174 and spacer 12.7, when delivered as an AAV vector, were able to edit the target stop cassette in mNPCs. AAV.34 and AAV.37 were generated with transgene constructs 34 (with a poly(A) of 186 nucleotides and a total transgene length of 4565 nucleotides) and 37 (with a poly(A) of 208 nucleotides and a total transgene length of 4619 nucleotides), respectively. Each vector displayed dose-dependent editing at the target locus, and AAV.34, which contains a shorter poly(A) signal, had approximately 75% of the editing potency of AAV.37 for both doses.

Under the conditions of the experiments, the results demonstrate that the expression of small CRISPR proteins (such as CasX) can be modulated by poly(A) signals of varying lengths. Longer poly(A) sequences can be utilized in the AAV constructs for enhanced CasX activity, while shorter poly(A) sequences can be utilized in the AAV constructs to make more sequence space available for the inclusion of additional accessory elements within the AAV transgene.

### Example 7: Small CRISPR Protein Potency is Modulated by the Position of the Regulatory Elements in the AAV Vector

Orientation (forward or reverse) and position (upstream or downstream of CRISPR gene) of regulatory elements such as the gRNA promoter and guide scaffold complex can modulate underlying expression of small CRISPR protein and overall editing efficiency of CRISPR systems in AAV vectors. The goal of these experiments was to assess the best orientation and position of regulatory elements within the AAV genome to enhance the potency of small CRISPR proteins and guide RNA.

### Materials and Methods:

AAV vector production and QC, nucleofection, AAV viral production and editing level assessment in mNPTC-tdT cells by FACS were conducted as described in Example 1.

### Results:

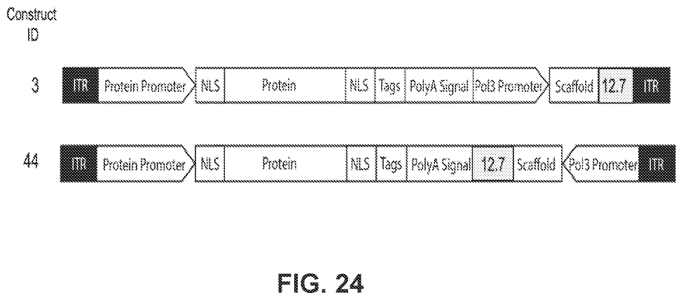

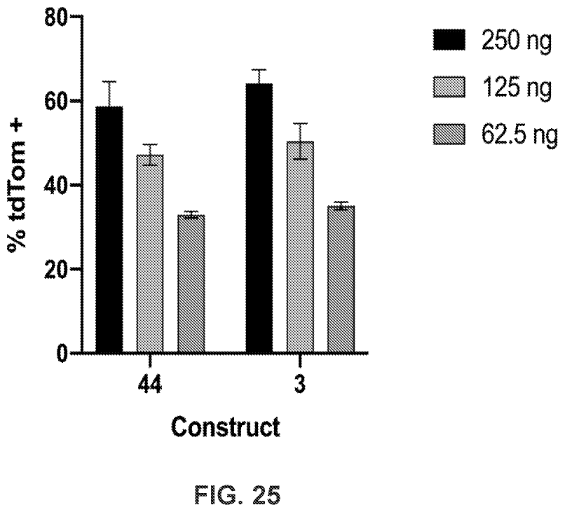

Construct 44 (configuration shown in FIG. 24, second from top) contains a Pol III promoter driving expression of guide scaffold 174 and spacer 12.7 in the reverse orientation of construct 3 (top configuration in FIG. 15). FIG. 25 demonstrates that construct 44, when delivered by nucleofection of an AAV transgene plasmid, modifies the target stop cassette in mNPCs similarly to construct 3 at in a dose-dependent manner.

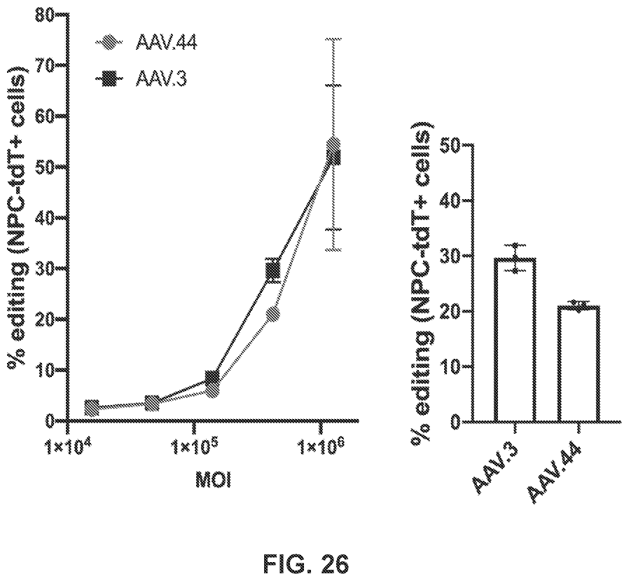

FIG. 26 shows that construct 44, delivered as an AAV vector, edits the target stop cassette in mNPCs, further supporting the utility of this construct. AAV.3 and AAV.44 were generated with transgene constructs 3 and 44, respectively. Each vector displayed dose-dependent editing at the target locus (FIG. 26, left panel, in which the vector was assayed using 3-fold dilutions). FIG. 26, right panel, shows editing results at an MOI of 3×10⁵, in which AAV.44 had 60% of the editing potency of the original configuration of vector AAV.3.

This experiment demonstrates that the orientation of parts within the AAV genome can be varied, yet result in sufficient expression of the CRISPR proteins and the guide RNA. This shows that specific orientations or positions of the regulatory elements relative to the encoded protein or RNA components may allow controlled modulation of expression in CasX-packaging AAV constructs that contain one or multiple guides.

### Example 8: Small CRISPR Protein Potency is Enhanced by Inclusion of Additional Regulatory Elements in the AAV Vector that are not Possible with a Larger Protein

The purpose these experiments was to demonstrate that transcriptional levels mediated by AAV vectors delivering small CRISPR proteins (such as CasX) can be enhanced by inclusion of different regulatory elements (intronic sequences, enhancers, etc.) that conventionally do not fit in AAV vectors expressing large transgene (e.g., spCas9) plasmids.

### Materials and Methods:

Cloning and QC: A 4-part Golden Gate Assembly consisting of a pre-digested AAV backbone, small CRISPR protein-encoding DNA, and flanking 5′ and 3′ DNA sequences was used to generate AAV-cis plasmid as described in Example 1. 5′ sequences contain enhancer, protein promoter and N-terminal NLS, while 3′ sequences contain C-terminal NLS, WPRE, poly(A) signal, RNA promoter and guide RNA containing spacer 12.7. 5′ and 3′ parts were ordered as gene fragments from Twist, PCR-amplified, and assembled and assembled into AAV vectors. Cloning and plasmid QC, nucleofection, and FACS methods were conducted as described in Example 1.

Enhancement in editing by the inclusion of post-translation regulatory element (PTRE) 1, 2, 3 in the AAV cis plasmid 3 was tested in combination of different promoters driving expression of CasX. A first set of promoters were tested; transgene plasmids 4, 35, 36 37, transgene plasmid 5, 38, 39, 40 and transgene plasmids 6, 42, 43 have the CasX protein expression driven by the CMV, UbC, EFS, CMV-s promoters, respectively. A second set of constructs tested included PTREs between the protein and poly(A) sequences and were generated with the promoter Jet, JetUsp compared to UbC promoter (transgene 58, 72, 73, 74; transgene 59, 75, 76, 77 and transgene 53, 80 and 81 respectively) driving expression of CasX. The sequences of the PTRE are listed in Table 12, and enhancer plus promoter sequences are listed in Table 13. The sequences of the additional components of the AAV constructs, with the exception of sequences encoding the CasX (Table 21) and the one or more gRNA (Tables 18 and 19), are listed in Table 26.

TABLE 12
Constructs and sequences of post-transcription elements tested on base
construct ID 4, 5, 6, 53, 58, and 59

| SEQ ID | Construct | | | Size |
|---|---|---|---|---|
| NO: | ID | PTRE | SEQUENCE | (bp) |
| 40431 | 35, 38, | 1 | AATCAACCTCTGGATTACAAAATTTGTGAAA | 598 |
| | 72, 75 | | GATTGACTGGTATTCTTAACTATGTTGCTCCT | |
| | | | TTTACGCTATGTGGATACGCTGCTTTAATGCC | |
| | | | TTTGTATCATGCTATTGCTTCCCGTATGGCTT | |
| | | | TCATTTTCTCCTCCTTGTATAAATCCTGGTTG | |
| | | | CTGTCTCTTTATGAGGAGTTGTGGCCCGTTGT | |
| | | | CAGGCAACGTGGCGTGGTGTGCACTGTGTTT | |
| | | | GCTGACGCAACCCCCACTGGTTGGGGCATTG | |
| | | | CCACCACCTGTCAGCTCCTTTCCGGGACTTTC | |
| | | | GCTTTCCCCCTCCCTATTGCCACGGCGGAACT | |
| | | | CATCGCCGCCTGCCTTGCCCGCTGCTGGACA | |
| | | | GGGGCTCGGCTGTTGGGCACTGACAATTCCG | |
| | | | TGGTGTTGTCGGGGAAGCTGACGTCCTTTCC | |
| | | | ATGGCTGCTCGCCTGTGTTGCCACCTGGATTC | |
| | | | TGCGCGGGACGTCCTTCTGCTACGTCCCTTCG | |
| | | | GCCCTCAATCCAGCGGACCTTCCTTCCCGCG | |
| | | | GCCTGCTGCCGGCTCTGCGGCCTCTTCCGCGT | |
| | | | CTTCGCCTTCGCCCTCAGACGAGTCGGATCTC | |
| | | | CCTTTGGGCCGCCTCCCCGCCTG | |
| 40432 | 36, 39, | 2 | ATCGATAATCAACCTCTGGATTACAAAATTT | 593 |
| | 42, 73, 76, | | GTGAAAGATTGACTGGTATTCTTAACTATGTT | |
| | 80 | | GCTCCTTTTACGCTATGTGGATACGCTGCTTT | |
| | | | AATGCCTTTGTATCATGCTATTGCTTCCCGTA | |
| | | | TGGCTTTCATTTTCTCCTCCTTGTATAAATCCT | |
| | | | GGTTGCTGTCTCTTTATGAGGAGTTGTGGCCC | |
| | | | GTTGTCAGGCAACGTGGCGTGGTGTGCACTG | |
| | | | TGTTTGCTGACGCAACCCCCACTGGTTGGGG | |
| | | | CATTGCCACCACCTGTCAGCTCCTTTCCGGGA | |
| | | | CTTTCGCTTTCCCCCTCCCTATTGCCACGGCG | |
| | | | GAACTCATCGCCGCCTGCCTTGCCCGCTGCTG | |
| | | | GACAGGGGCTCGGCTGTTGGGCACTGACAAT | |
| | | | TCCGTGGTGTTGTCGGGGAAATCATCGTCCTT | |
| | | | TCCTTGGCTGCTCGCCTGTGTTGCCACCTGGA | |
| | | | TTCTGCGCGGGACGTCCTTCTGCTACGTCCCT | |
| | | | TCGGCCCTCAATCCAGCGGACCTTCCTTCCCG | |
| | | | CGGCCTGCTGCCGGCTCTGCGGCCTCTTCCGC | |
| | | | GTCTTCGCCTTCGCCCTCAGACGAGTCGGATC | |
| | | | TCCCTTTGGGCCGCCTCCCC | |
| 40433 | 37, 40, | 3 | GATAATCAACCTCTGGATTACAAAATTTGTG | 247 |
| | 43, 74, | | AAAGATTGACTGGTATTCTTAACTATGTTGCT | |
| | 77, 81 | | CCTTTTACGCTATGTGGATACGCTGCTTTAAT | |
| | | | GCCTTTGTATCATGCTATTGCTTCCCGTATGG | |
| | | | CTTTCATTTTCTCCTCCTTGTATAAATCCTGGT | |
| | | | TAGTTCTTGCCACGGCGGAACTCATCGCCGC | |
| | | | CTGCCTTGCCCGCTGCTGGACAGGGGCTCGG | |
| | | | CTGTTGGGCACTGACAATTCCGTGG | |

TABLE 13
Enhancer elements and sequences tested in combination with the CMV core promoter

| SEQ ID | | | Core | | Size |
|---|---|---|---|---|---|
| NO: | ID | Enhancer | promoter | Sequence | (bp) |
| 40434 | 3 | CMV | CMV | GACATTGATTATTGACTAGTTATTAATAGTA | 584 |
| | | | | ATCAATTACGGGGTCATTAGTTCATAGCCCA | |
| | | | | TATATGGAGTTCCGCGTTACATAACTTACGG | |
| | | | | TAAATGGCCCGCCTGGCTGACCGCCCAACG | |
| | | | | ACCCCCGCCCATTGACGTCAATAATGACGTA | |
| | | | | TGTTCCCATAGTAACGCCAATAGGGACTTTC | |
| | | | | CATTGACGTCAATGGGTGGAGTATTTACGGT | |
| | | | | AAACTGCCCACTTGGCAGTACATCAAGTGTA | |
| | | | | TCATATGCCAAGTACGCCCCCTATTGACGTC | |
| | | | | AATGACGGTAAATGGCCCGCCTGGCATTAT | |
| | | | | GCCCAGTACATGACCTTATGGGACTTTCCTA | |
| | | | | CTTGGCAGTACATCTACGTATTAGTCATCGC | |
| | | | | TATTACCATGGTGATGCGGTTTTGGCAGTAC | |
| | | | | ATCAATGGGCGTGGATAGCGGTTTGACTCAC | |
| | | | | GGGGATTTCCAAGTCTCCACCCCATTGACGT | |
| | | | | CAATGGGAGTTTGTTTTGGCACCAAAATCAA | |
| | | | | CGGGACTTTCCAAAATGTCGTAACAACTCCG | |
| | | | | CCCCATTGACGCAAATGGGCGGTAGGCGTG | |
| | | | | TACGGTGGGAGGTCTATATAAGCAGAGCT | |
| 40435 | 64 | N/A | CMV | GTGATGCGGTTTTGGCAGTACATCAATGGGC | 204 |
| | | | | GTGGATAGCGGTTTGACTCACGGGGATTTCC | |
| | | | | AAGTCTCCACCCCATTGACGTCAATGGGAGT | |
| | | | | TTGTTTTGGCACCAAAATCAACGGGACTTTC | |
| | | | | CAAAATGTCGTAACAACTCCGCCCCATTGAC | |
| | | | | GCAAATGGGCGGTAGGCGTGTACGGTGGGA | |
| | | | | GGTCTATATAAGCAGAGCT | |
| 40436 | 65 | Syn 1 | CMV | AAGATGCGTCAATTAATTTGCGTCAATTTGC | 414 |
| | | | | GCTCAATTTGCGTCAATCTTGCTGTCATTTG | |
| | | | | CGTCAATTTGCGTCAATATGCGTCAATATAT | |
| | | | | GCGTCAATTCGAATTCGCACTAATGATGACT | |
| | | | | AATGGTGGCTAATGGTGACTAATGGTGACA | |
| | | | | ATGCGTGACTAATGGTGATAATGAGTGCAT | |
| | | | | ATGGTGACTAATGGTGACTAATGGTGGTGAT | |
| | | | | GCGGTTTTGGCAGTACATCAATGGGCGTGG | |
| | | | | ATAGCGGTTTGACTCACGGGGATTTCCAAGT | |
| | | | | CTCCACCCCATTGACGTCAATGGGAGTTTGT | |
| | | | | TTTGGCACCAAAATCAACGGGACTTTCCAAA | |
| | | | | ATGTCGTAACAACTCCGCCCCATTGACGCAA | |
| | | | | ATGGGCGGTAGGCGTGTACGGTGGGAGGTC | |
| | | | | TATATAAGCAGAGCT | |
| 40437 | 66 | NPC5 | CMV | TGGGACAGAAAACGAGAAACCAGGGTTGTC | 314 |
| | | | | AGCGGGGCCCGCGCCGGCCGCCCCTTGGCC | |
| | | | | CGCGGGATACCCCGGGCGCCCAGTGCCCAG | |
| | | | | GCCGGGCAGGCGGCACTCACGTGATGCGGT | |
| | | | | TTTGGCAGTACATCAATGGGCGTGGATAGC | |
| | | | | GGTTTGACTCACGGGGATTTCCAAGTCTCCA | |
| | | | | CCCCATTGACGTCAATGGGAGTTTGTTTTGG | |
| | | | | CACCAAAATCAACGGGACTTTCCAAAATGT | |
| | | | | CGTAACAACTCCGCCCCATTGACGCAAATG | |
| | | | | GGCGGTAGGCGTGTACGGTGGGAGGTCTAT | |
| | | | | ATAAGCAGAGCT | |
| 40438 | 67 | NPC7 | CMV | CGGAAGCGAGGGTGTCGCTCGCCCCCGGGC | 324 |
| | | | | CCGGGTCCGCCCCGCTCCGAGGCCTGCTCGG | |
| | | | | AAGAAAGACCTCGGTGCGCAGTTCTCGTCG | |
| | | | | CGCTCCCACACCTGGTCCGCCCAGTCGGAGT | |
| | | | | GATGCGGTTTTGGCAGTACATCAATGGGCGT | |
| | | | | GGATAGCGGTTTGACTCACGGGGATTTCCAA | |
| | | | | GTCTCCACCCCATTGACGTCAATGGGAGTTT | |
| | | | | GTTTTGGCACCAAAATCAACGGGACTTTCCA | |
| | | | | AAATGTCGTAACAACTCCGCCCCATTGACGC | |
| | | | | AAATGGGCGGTAGGCGTGTACGGTGGGAGG | |
| | | | | TCTATATAAGCAGAGCT | |
| 40439 | 68 | NPC127 | CMV | GTGATGCGGTTTTGGCAGTACATCAATGGGC | 304 |
| | | | | GTGGATAGGCGGGCCGGGAGCGAGGGAGGC | |
| | | | | GGCGCCGGGGGACGCGCCGGGCTCGGCCTG | |
| | | | | GCGACCGTTGCCCGCTCGCGTCCATCCATCC | |
| | | | | ATTCATTCGGGCGGCAGCGGTTTGACTCACG | |
| | | | | GGGATTTCCAAGTCTCCACCCCATTGACGTC | |
| | | | | AATGGGAGTTTGTTTTGGCACCAAAATCAAC | |
| | | | | GGGACTTTCCAAAATGTCGTAACAACTCCGC | |
| | | | | CCCATTGACGCAAATGGGCGGTAGGCGTGT | |
| | | | | ACGGTGGGAGGTCTATATAAGCAGAGCT | |
| 40440 | 69 | NPC190 | CMV | AGGCGGGCCGGGAGCGAGGGAGGCGGCGC | 364 |
| | | | | CGGGGGACGCGCCGGGCTCGGCCTGGCGAC | |
| | | | | CGTTGCCCGCTCGCGTCCATCCATCCATTCA | |
| | | | | TTCGGGCGGCGTGATGCGGTTTTGGCAGTAC | |
| | | | | ATCAATGGGCGTGGATAGCGGTTTGACTCAC | |
| | | | | GGGGATTTCCAAGTCTCCACCCCATTGACGT | |
| | | | | CAATGGGAGTTTGTTTTGGCACCAAAATCAA | |
| | | | | CGGGACTTTCCAAAATGTCGTAACAACTCCG | |
| | | | | CCCCATTGACGCAAATGGGCGGTAGGCGTG | |
| | | | | TACGGTGGGAGGTCTATATAAGCAGAGCT | |
| 40441 | 70 | NPC249 | CMV | CCTTCCCCTCCAGTGCTCCTCGGAGCCCTTC | 274 |
| | | | | CCTATACTTCCTCCAAGCTCCACCCTCGATC | |
| | | | | AGCCCTGCGTGATGCGGTTTTGGCAGTACAT | |
| | | | | CAATGGGCGTGGATAGCGGTTTGACTCACG | |
| | | | | GGGATTTCCAAGTCTCCACCCCATTGACGTC | |
| | | | | AATGGGAGTTTGTTTTGGCACCAAAATCAAC | |
| | | | | GGGACTTTCCAAAATGTCGTAACAACTCCGC | |
| | | | | CCCATTGACGCAAATGGGCGGTAGGCGTGT | |
| | | | | ACGGTGGGAGGTCTATATAAGCAGAGCT | |
| 40442 | 71 | NPC286 | CMV | AGAGGTGGTGGGGCTGAGCCGAGGTGGGGC | 354 |
| | | | | CGTGGCCAGGGGGAGGGGGTGCTAGGCCGG | |
| | | | | AAGGGGCTGCAGCCGAGGGTGGCCCTGATT | |
| | | | | TTGTGGCCGGCCAGGAGCGAAGGGGTCCCT | |
| | | | | TTCTGTCCCCTGAGCACCGTCGCCTCCTTTGT | |
| | | | | GATGCGGTTTTGGCAGTACATCAATGGGCGT | |
| | | | | GGATAGCGGTTTGACTCACGGGGATTTCCAA | |
| | | | | GTCTCCACCCCATTGACGTCAATGGGAGTTT | |
| | | | | GTTTTGGCACCAAAATCAACGGGACTTTCCA | |
| | | | | AAATGTCGTAACAACTCCGCCCCATTGACGC | |
| | | | | AAATGGGCGGTAGGCGTGTACGGTGGGAGG | |
| | | | | TCTATATAAGCAGAGCT | |

### Results:

The effects of PTREs on transgene expression were assessed by cloning 3 enhancer sequences (PTRE1, PTRE2, and PTR3) into an AAV-cis plasmid (construct 3) and construct plasmids containing shorter protein promoters (constructs 4, 5, 6, 53, 57 and 58 contain 400, 234, 335, 400, 164 and 326 bp promoter sequences, respectively).

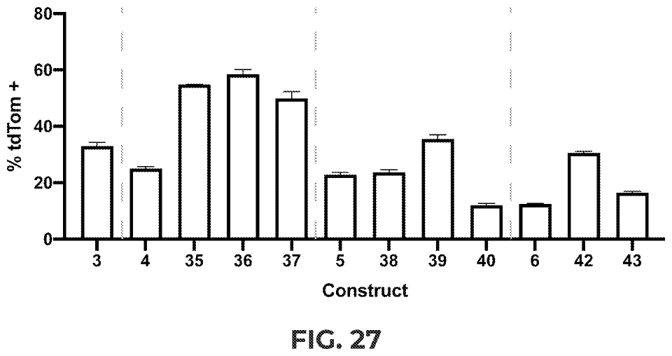

AAV-cis plasmid activity was first confirmed by nucleofection in mNPC-tdt cells. For each vector, addition of PTRE enhanced editing activity at various levels (FIG. 27). Table 14 provides the lengths of promoter and PTREs. The addition of PTRE2 to the transgene cassette showed the highest CasX editing activity enhancement, with a 2-fold increase in editing levels for construct 36 compared to construct 4 (58.5% vs 25%), a 1.5-fold increase for construct 39 (35.4% vs 22.9%) compared to construct 5 and a 3-fold increase for construct 42 compared to construct 6 (30.5% vs 12%). The shortest enhancer sequence, PTRE3, also increased protein activity at various levels among construct 37 and 43 compared to other vectors.

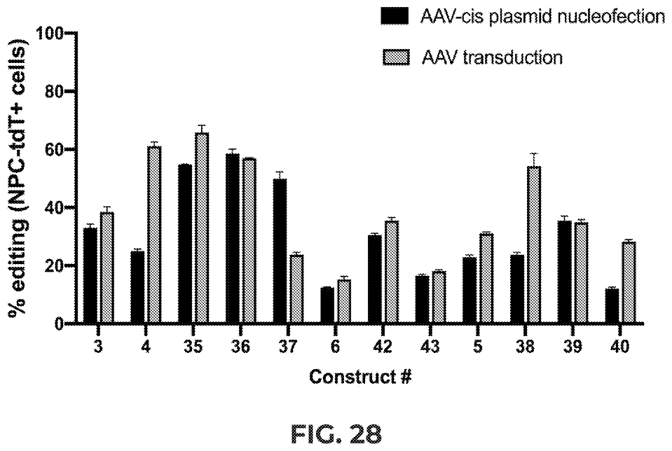

Improvements in editing levels were also observed when constructs were packaged into AAV. Inclusion of PTRE2 in transgene increased editing across the AAV vectors in a similar manner. Trends in on-target editing observed in mNPCs with the AAV infection generally correlated with the AAV plasmid nucleofection data set (FIG. 28).

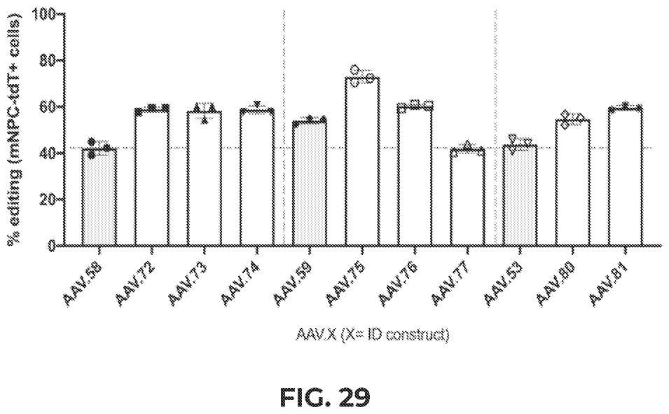

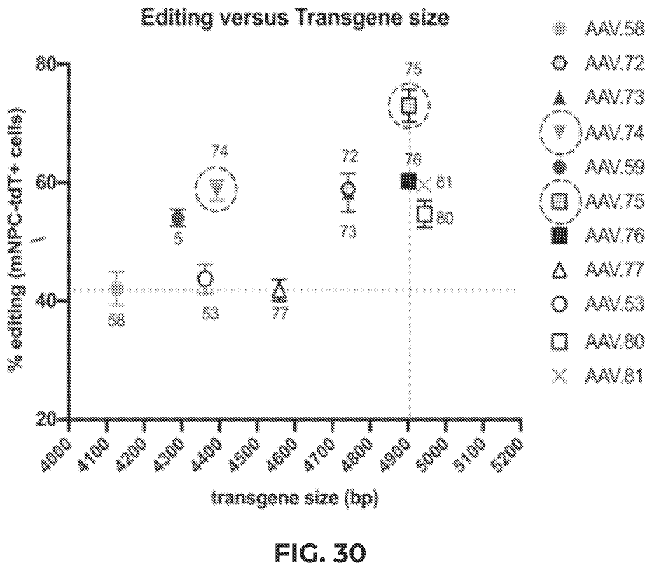

The trend was confirmed by testing another set of promoters with inclusion of these enhancer sequences. Across all AAV vectors tested, constructs including a PTRE1 and PTRE2 in genomes yielded an average 1.5-fold increase compared to base vectors (FIG. 29). Unique combinations of short promoter and these post-transcriptional sequences led to the identification of vectors with increased editing levels with shorter promoter (e.g., AAV.74), which represents an advantage both for AAV manufacturing being under the carrying capacity limit of AAV, and allows for inclusion of more regulatory elements and CRISPR elements (e.g., guides) (FIG. 30).

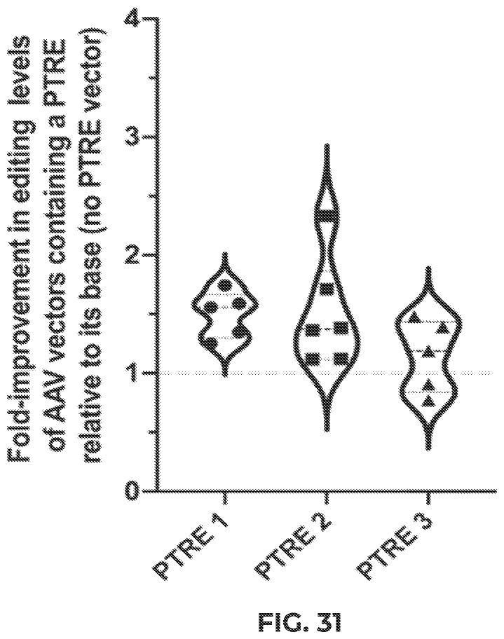

The results also demonstrate that inclusion of PTRE1 in the transgene plasmid improved editing levels across all promoters evaluated (FIG. 31), with less variability, while PTRE2 yielded the highest transgene improvement but with more variability across the promoters tested.

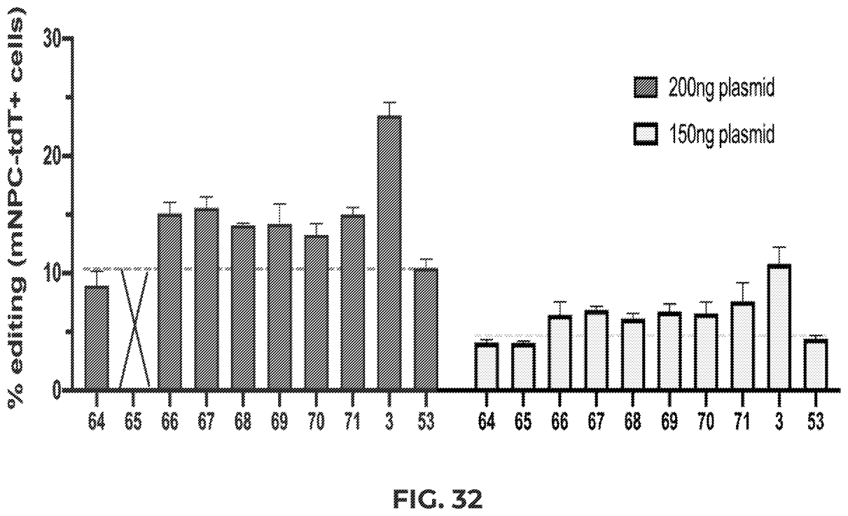

Several constructs with tissue-specific neuronal enhancers upstream of a single constitutive promoter were tested. In this assay, 7 neuronal enhancer sequences (constructs 65-72) were cloned into a single AAV-cis plasmid (64) harboring a core CMV promoter and all demonstrated improved editing via nucleofection over base construct 64 (FIG. 32). These constructs also outperformed construct 53, which contains a UbC promoter but did not outperform construct 3 which harbors the full CMV promoter (CMV enhancer+CMV core promoter).

TABLE 14
Constructs with or without PTREs and indicated sequence lengths
Construct
(Sequence length indicated below)

| | 3 | 4 | 35 | 36 | 37 | 5 | 38 | 39 | 40 | 6 | 42 | 43 |
|---|---|---|---|---|---|---|---|---|---|---|---|---|
| Promoter | 584 | 400 | | | | 234 | | | | 335 | | |
| Length | | | | | | | | | | | | |
| PTRE 1 | — | — | 592 | — | — | — | 592 | — | — | — | — | — |
| PTRE 2 | — | — | — | 593 | — | — | — | 593 | — | — | 593 | — |
| PTRE 3 | — | — | — | — | 247 | — | — | — | 247 | — | — | 247 |
| AAV | 4550 | 4349 | 4964 | 4965 | 4619 | 4183 | 4798 | 4799 | 4453 | 4284 | 4900 | 4554 |
| transgene | | | | | | | | | | | | |

The results demonstrate that use of small promoters in the AAV transgene constructs permits the inclusion of additional accessory elements. These additional accessory elements, such as post-transcriptional regulatory elements to AAV-transgenes expressing CasX under the control of short but strong promoter sequences enable increased CasX expression and on-target editing while reducing cargo size such that all components can be incorporated into a single AAV vector.

### Example 9: Small CRISPR Protein Potency is Enhanced by Inclusion and Combining Additional Regulatory Elements in the AAV Vector

The goal of these experiments was to demonstrate that CRISPR protein and gRNA complex-mediated editing can be enhanced in an all-in-one single AAV vector that can include more than one guide RNA. Furthermore, experiments were conducted to show that the inclusion and combination of many regulatory elements can enhance potency and that larger AAV genomes having more regulatory sequence yield greater editing activity. The length of accessory and regulatory sequence that is possible to include with the CasX system in an AAV transgene is beyond what is possible with traditionally used CRISPR proteins, which are limited by the length of the larger Cas proteins, such as Cas9.

### Materials and Methods:

Plasmid cloning, QC, and nucleofection were conducted as described in Example 1.

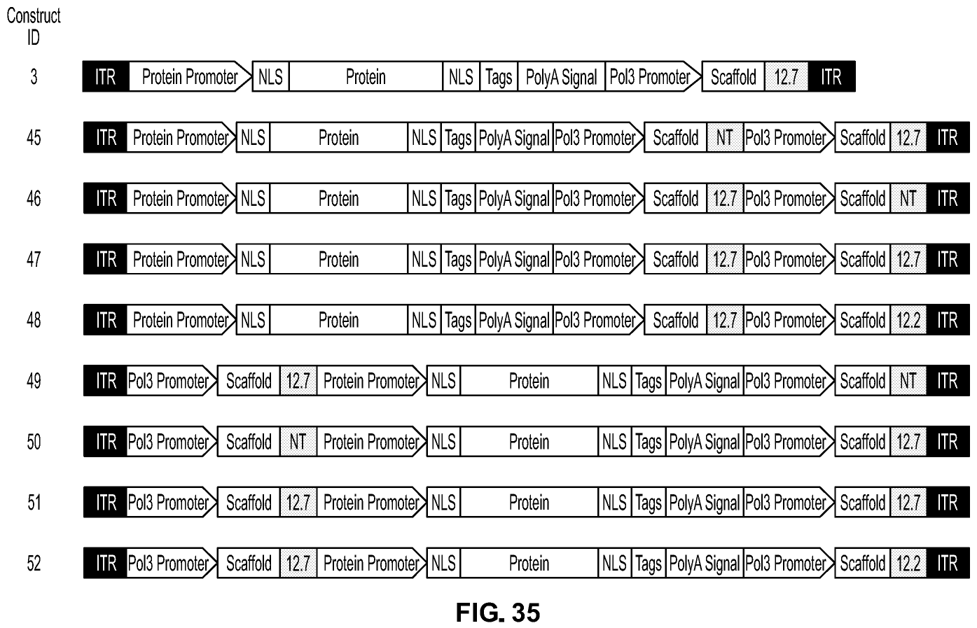

Orientations of multiple RNA transcriptional unit blocks (FIG. 35) referred as “guide RNA stacks” (each stack composed of a sgRNA scaffold-spacer 174.12.7, 1.74.12.2 or 174.NT driven by the U6 promoter) were investigated by cloning two guide RNA stacks in a tail to tail orientation (plasmid ID 45-49) on the 3′ end of the poly(A) or still in the same transcriptional orientation than the CasX protein/promoter, one on each side of the protein (plasmid ID=50-52). Pentagon shaped boxes for protein promoter and Pol III promoter depict orientation of transcription (tapered point; 5′ to 3′ or 3′ to 5′ orientation). Spacer sequences are 12.2 (TATAGCATACATTATACGAA SEQ ID NO: 40807)); 12.7 (CTGCATTCTAGTTGTGGTTT (SEQ ID NO: 40800)); and NT (GGGTCTTCGAGAAGACCC (SEQ ID NO: 40505)). AAV vector production and titering were conducted as described in Example 1. AAV transduction and editing assessment via FACs sorting were conducted as described in Example 1.

### Results:

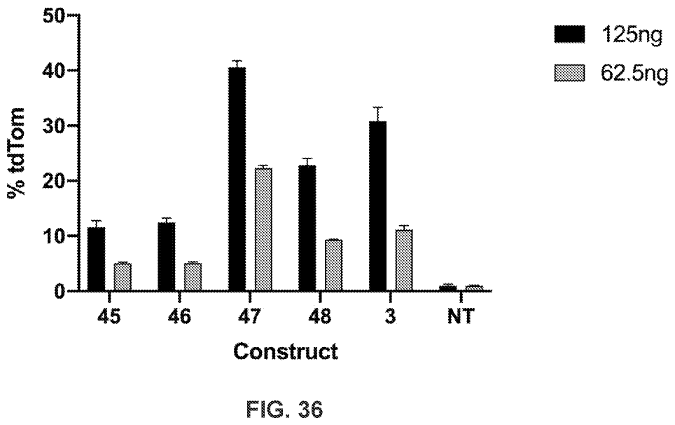

FIG. 33 is a schematic of the architecture of the constructs, showing how the guide RNA components were combined in the various constructs (architecture 1 and architecture 2). FIG. 34 shows additional configurations. The results of the editing assay portrayed in FIG. 36 demonstrate that the constructs delivered as AAV transgene plasmids to mNPCs in architecture 1 edit with enhanced potency. Different combinations of spacers and non-targeting spacers demonstrate that each individual guide RNA is active, although, architectures with one targeting spacer and one non-targeting spacer (constructs 45 and 46) yielded approximately 18% lower editing levels. Certain combinations of targeting spacers yielded increased efficacy. Spacer 12.7 with the sequence of CTGCATTCTAGTTGTGGTTT (SEQ ID NO: 40800), in combination with spacer 12.2 (construct 48), with the sequence of TATAGCATACATTATACGAA (SEQ ID NO: 40807), edited with significant potency in guide RNA architecture 1, while two sets of 12.7 (construct 47) edited with 10% greater potency than the single guide architecture of construct 3. 125 and 62.5 ng of each CasX construct was nucleofected in mNPCs, and editing was assessed by FACS 5 days post-transfection. Data are presented as mean±SEM for n=3 replicates.

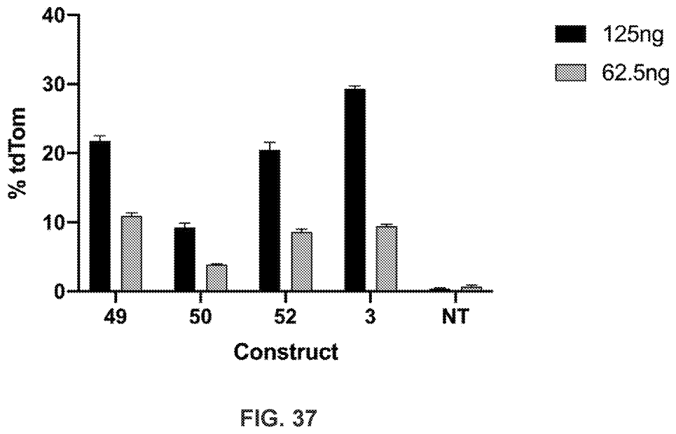

The results of FIG. 37 show that guide RNA stack architecture 2 (see FIG. 33) delivered as AAV transgene plasmid to mNPCs also edit the target nucleic acid. 125 and 62.5 ng of each CasX construct was nucleofected in mNPCs, and editing was assessed by FACS 5 days post-transfection. Data are presented as mean±SEM for n=3 replicates.

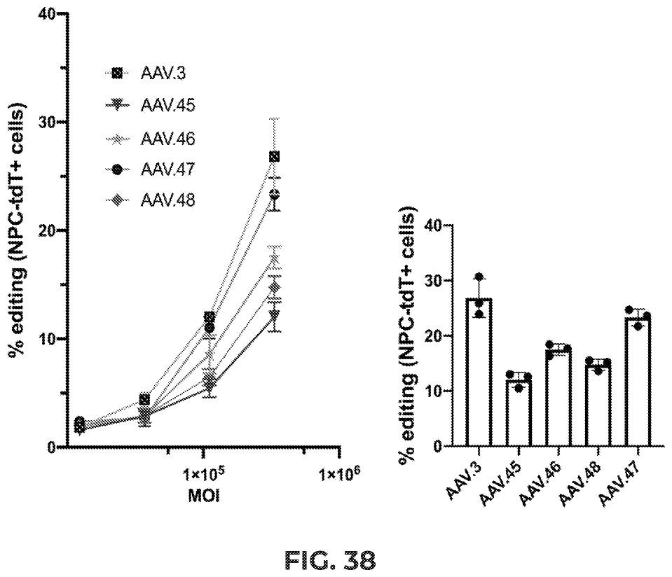

The results of FIG. 38 show that constructs 3, 45, 46, 47, and 48 delivered as AAVs in guide RNA architecture 1 edit the target stop cassette in mNPCs. AAV.3, AAV.45, AAV.46, AAV.47 and AAV.48 were generated with transgene constructs 3 and 45, 46, 47 and 48, respectively. Each vector displayed dose-dependent editing at the target locus (FIG. 38, left panel). At an MOI of 3e5, AAV.47 had <5% less potency than the original orientation vector AAV.3 (FIG. 38, right panel).

These experiments demonstrate the feasibility of the use of multiple guide RNAs in combination with the full Cas protein sequence in one AAV genome, which was previously un-achievable with the use of larger CRISPR proteins, such as Cas9, due to the packaging constraints of the AAV capsid. Furthermore, these experiments also show that multiple guide RNAs in an all-in-one vector also retain the ability to edit the target nucleic acid.

### Example 10: Small CRISPR Protein Potency is Enhanced by Nuclear Localization Sequence (NLS) Choice

Experiments were conducted to determine whether alteration of the nuclear localization sequence (NLS) utilized in constructs can modulate editing outcomes in the AAV setting. In the larger context of optimizing the AAV for editing with CasX proteins, this initial screen served as a first attempt to determine which NLS should be used in constructs moving forward.

### Materials and Methods.

Cloning and QC: AAV vectors were cloned using a 4-part Golden Gate Assembly consisting of a pre-digested AAV backbone, small CRISPR protein-encoding DNA, and flanking 5′ and 3′ DNA sequences. 5′ sequences contain enhancer, protein promoter and N-terminal NLS, while 3′ sequences contain C-terminal NLS, WPRE, poly(A) signal, RNA promoter and guide RNA containing spacer 12.7. 5′ and 3′ parts were ordered as gene fragments from Twist, PCR-amplified, and assembled into AAV vectors through cyclical Golden Gate reactions using T4 Ligase and BbsI. NLS sequences are presented in Tables 15 and 16.

Methods for the assembly and QC of AAV vectors and nucleofection were conducted as described in Example 1. The sequences of the additional components of the AAV constructs, with the exception of sequences encoding the CasX (Table 21) and the one or more gRNA (Tables 18 and 19), are listed in Table 26.

TABLE 15
5′ NLS sequences

| SEQ ID | | 5′ NLS |
|---|---|---|
| NO: | NLS Amino Acid Sequence* | ID |
| 40443 | PKKKRKVSR | 1 |
| 40444 | PKKKRKVGGSPKKKRKVGGSPKKKRKVGGSPKKKRKVSR | 2 |
| 40445 | PKKKRKVGGSPKKKRKVGGSPKKKRKVGGSPKKKRKVGGSPKKKRKV | 3 |
| | GGSPKKKRKVSR | |
| 40446 | PAAKRVKLDSR | 4 |
| 40447 | PAAKRVKLDGGSPAAKRVKLDSR | 5 |
| 40448 | PAAKRVKLDGGSPAAKRVKLDGGSPAAKRVKLDGGSPAAKRVKLDSR | 6 |
| 40449 | PAAKRVKLDGGSPAAKRVKLDGGSPAAKRVKLDGGSPAAKRVKLDGGS | 7 |
| | PAAKRVKLDGGSPAAKRVKLDSR | |
| 40450 | KRPAATKKAGQAKKKKSR | 8 |
| 40451 | KRPAATKKAGQAKKKKGGSKRPAATKKAGQAKKKKSR | 9 |
| 40452 | PAAKRVKLDGGSPKKKRKVSR | 10 |
| 40453 | PAAKKKKLDGGSPKKKRKVSR | 11 |
| 40454 | PAAKKKKLDSR | 12 |
| 40455 | PAAKKKKLDGGSPAAKKKKLDGGSPAAKKKKLDSR | 13 |
| 40456 | PAAKKKKLDGGSPAAKKKKLDGGSPAAKKKKLDGGSPAAKKKKLDSR | 14 |
| 40457 | PAKRARRGYKCSR | 15 |
| 40458 | PAKRARRGYKCGSPAKRARRGYKCSR | 16 |
| 40459 | PRRKREESR | 17 |
| 40443 | PYRGRKESR | 18 |
| 40444 | PLRKRPRRSR | 19 |
| 40445 | PLRKRPRRGSPLRKRPRRSR | 20 |
| 40446 | PAAKRVKLDGGKRTADGSEFESPKKKRKVGGS | 21 |
| 40447 | PAAKRVKLDGGKRTADGSEFESPKKKRKVPPPPG | 22 |
| 40448 | PAAKRVKLDGGKRTADGSEFESPKKKRKVGIHGVPAAPG | 23 |
| 40449 | PAAKRVKLDGGKRTADGSEFESPKKKRKVGGGSGGGSPG | 24 |
| 40450 | PAAKRVKLDGGKRTADGSEFESPKKKRKVPGGGSGGGSPG | 25 |
| 40451 | PAAKRVKLDGGKRTADGSEFESPKKKRKVAEAAAKEAAAKEAAAKAPG | 26 |
| 40452 | PAAKRVKLDGGKRTADGSEFESPKKKRKVPG | 27 |
| 40453 | PAAKRVKLDGGSPKKKRKVGGS | 28 |
| 40454 | PAAKRVKLDPPPPKKKRKVPG | 29 |
| 40455 | PAAKRVKLDPG | 30 |
| 40456 | PAAKRVKLDGGGSGGGSGGGS | 31 |
| 40457 | PAAKRVKLDPPP | 32 |
| 40458 | PAAKRVKLDGGGSGGGSGGGSPPP | 33 |
| 40459 | PKKKRKVPPP | 34 |
| 40460 | PKKKRKVGGS | 35 |

*Sequences in bold are NLS, while unbolded sequences are linkers.

TABLE 16
3′ NLS sequences

| SEQ ID | | 3′ NLS |
|---|---|---|
| NO: | NLS Amino Acid Sequence* | ID |
| 40461 | GSPKKKRKV | 1 |
| 40462 | GSPKKKRKVGGSPKKKRKVGGSPKKKRKVGGSPKKKRKV | 2 |
| 40463 | GSPKKKRKVGGSPKKKRKVGGSPKKKRKVGGSPKKKRKVGGSPKKK | 3 |
| | RKVGGSPKKKRKV | |
| 40464 | GSPAAKRVKLD | 4 |
| 40465 | GSPAAKRVKLDGGSPAAKRVKLD | 5 |
| 40466 | GSPAAKRVKLDGGSPAAKRVKLDGGSPAAKRVKLDGGSPAAKRVKLD | 6 |
| 40467 | GSPAAKRVKLDGGSPAAKRVKLDGGSPAAKRVKLDGGSPAAKRVKLD | 7 |
| | GGSPAAKRVKLDGGSPAAKRVKLD | |
| 40468 | GSKRPAATKKAGQAKKKK | 8 |
| 40469 | KRPAATKKAGQAKKKKGGSKRPAATKKAGQAKKKK | 9 |
| 40470 | GSPAAKRVKLGGSPAAKRVKLGGSPKKKRKVGGSPKKKRKV | 10 |
| 40471 | GSKLGPRKATGRWGS | 77 |
| 40472 | GSKRKGSPERGERKRHWGS | 12 |
| 40473 | GSPKKKRKVGSGSKRPAATKKAGQAKKKKLE | 73 |
| 40474 | GPKRTADSQHSTPPKTKRKVEFEPKKKRKV | 14 |
| 40475 | GGGSGGGSKRTADSQHSTPPKTKRKVEFEPKKKRKV | 15 |
| 40476 | AEAAAKEAAAKEAAAKAKRTADSQHSTPPKTKRKVEFEPKKKRKV | 16 |
| 40477 | GPPKKKRKVGGSKRTADSQHSTPPKTKRKVEFEPKKKRKV | 17 |
| 40478 | GPAEAAAKEAAAKEAAAKAPAAKRVKLD | 18 |
| 40479 | GPGGGSGGGSGGGSPAAKRVKLD | 19 |
| 40480 | GPPKKKRKVPPPPAAKRVKLD | 20 |
| 40481 | GPPAAKRVKLD | 21 |
| 40482 | GSPKKKRKV | 22 |
| 40483 | GSPAAKRVKLD | 23 |
| 40484 | VGSKRPAATKKAGQAKKKK | 24 |
| 40485 | TGGGPGGGAAAGSGSPKKKRKVGSGSKRPAATKKAGQAKKKKLE | 25 |
| 40486 | TGGGPGGGAAAGSGSPKKKRKVGSGSKRPAATKKAGQAKKKKLE | 26 |
| 40487 | TGGGPGGGAAAGSGSPKKKRKVGSGS | 27 |
| 40488 | PPPPKKKRKVPPP | 28 |
| 40489 | GGSPKKKRKVPPP | 29 |
| 40490 | PPPPKKKRKV | 30 |
| 40491 | GGSPKKKRKV | 31 |
| 40492 | GGSPKKKRKVGGSGGSGGS | 32 |
| 40493 | GGSPKKKRKVGGSPKKKRKV | 33 |
| 40494 | GGSGGSGGSPKKKRKVGGSPKKKRKV | 34 |
| 40495 | VGGGSGGGSGGGSPAAKRVKLD | 35 |
| 40496 | VPPPPAAKRVKLD | 36 |
| 40497 | VPPPGGGSGGGSGGGSPAAKRVKLD | 37 |
| 40498 | VGGGSGGGSGGGSPAAKRVKLD | 38 |
| 40499 | VPPPPAAKRVKLD | 39 |
| 40500 | VPPPGGGSGGGSGGGSPAAKRVKLD | 40 |
| 40501 | VGSPAAKRVKLD | 41 |

AAV transduction and editing level assessment in mNPTC-tdT cells by FACS were conducted as described in Example 1.

### Results:

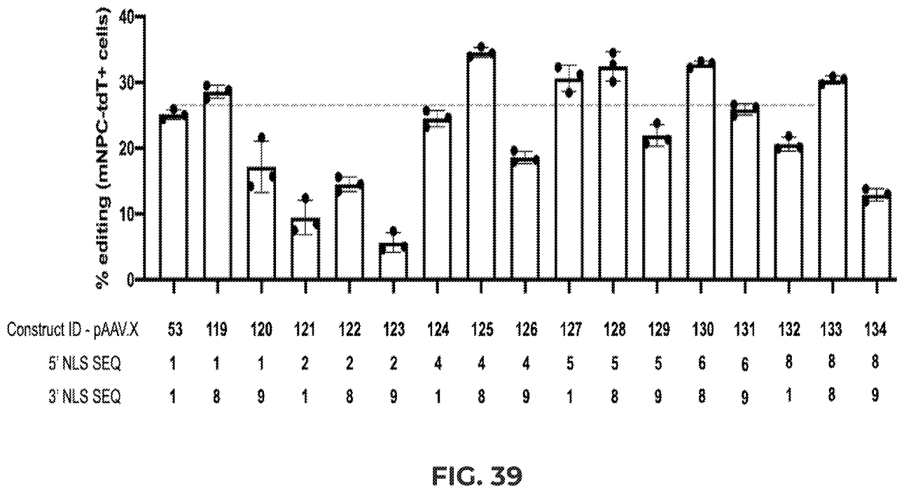

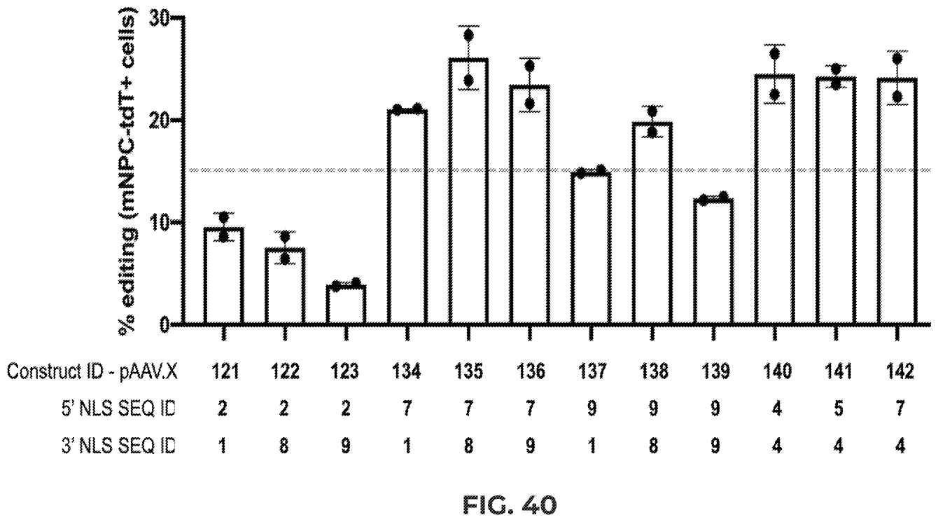

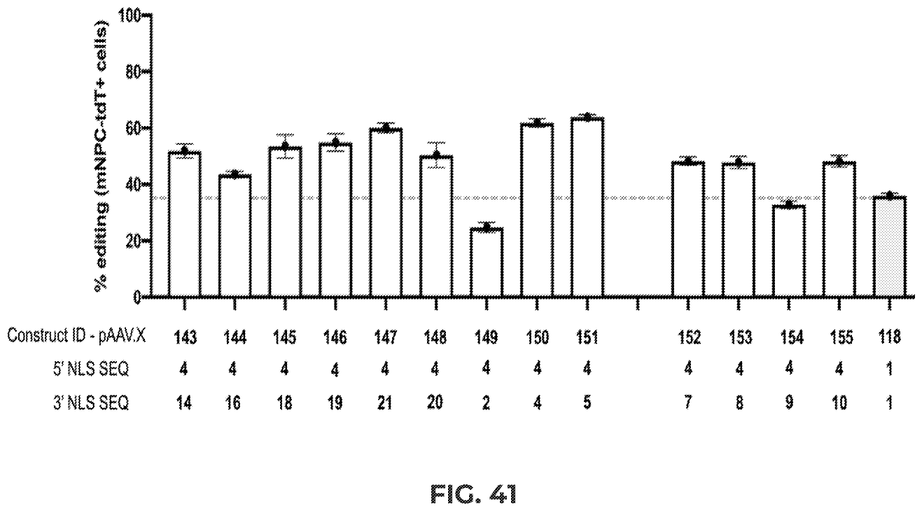

Initial plasmid nucleofection revealed that a number of NLS permutations displayed improved editing when compared to control (1×SV40 NLS on both the N- and C-termini). In particular, N-terminal variants containing Cmyc or Nucleoplasmin NLSs significantly outperformed SV40 NLS combinations (FIG. 39). This trend in N-terminal NLS variation was replicated in AAV transduction, where Cmyc and Nucleoplasmin NLS variants again outperformed SV40 NLS variants (FIG. 40). Finally, variations holding the Cmyc constant (FIG. 41) were tested, and the results demonstrate that the best constructs contained a Cmyc NLS on both the N- and C-terminals.

The data suggests that selecting the amino acid sequence of the NLS can enhance editing outcomes in the AAV setting. Specifically, N-terminal Cmyc-containing NLS variants showed a clear improvement compared to N-terminal SV40 NLS variants. In addition, C-terminal Cmyc and Nuc variants improve editing over SV40 NLS variants. Repetitions of the SV40 NLS seem to be deleterious for editing efficiency on both the N- and C-terminals.

### Example 11: Small CRISPR Protein Expression is Enhanced by Addition of Introns in the 5′ UTR

The goal of this experiment is to demonstrate that transcriptional levels mediated by AAV vectors delivering small CRISPR proteins (such as CasX) can be enhanced by inclusion of different regulatory elements such as intronic sequences taken from viral, mouse, or human genomes that conventionally do not fit in AAV vectors expressing large transgene (e.g., spCas9) plasmids.

### Methods:

A 4-part Golden Gate Assembly consisting of a pre-digested AAV backbone, small CRISPR protein-encoding DNA, and flanking 5′ and 3′ DNA sequences will be used to generate AAV-cis plasmid. 5′ sequences will contain protein promoters including UbC, JeT, CMV, CAG, CBH, hSyn, or other Pol2 promoter, intronic region, and N-terminal NLS, while 3′ sequences will contain C-terminal NLS, poly A signal, RNA promoter and guide RNA containing spacer 12.7. 5′ and 3′ parts will be PCR-amplified and assembled as described in Example 1 into. Cloning and plasmid QC, AAV viral production and editing level assessment in mNPTC-tdT cells by FACS will be conducted as described in Example 1. Non-limiting examples of intron sequences to be incorporated into the constructs are listed in Table 17.

Enhancement in editing by the inclusion of intron 36 (transgene plasmid 59) will be tested against transgene plasmid 58, which was the base construct not containing the intron. The rest of the introns are prophetic intron sequences that can be used in future constructs coding for CasX and have been derived from viral, mouse, and human origin.

TABLE 17
Intron sequences for incorporation into base construct 58

| | SEQ ID | | Size |
|---|---|---|---|
| Intron | NO: | Sequence | (bp) |
| 1 | 40599 | GTGGCCCAGGCAGGCAGACCCACCAGGGGTCCCTGAAGGCCAGCCCT | 54 |
| | | TGAGAAG | |
| 2 | 40600 | GTCATACAACTTTCCTGAAGTTGTATGACCTCTCTGAGCCTTAGTCT | 67 |
| | | CCTCGTTTGTAAAATGAGAG | |
| 3 | 40601 | GTAAGAGCATAGTGCACAGGACTGCTGGTGGCCAGGAGGCCCAGCCC | 62 |
| | | TGGATCTTCCTCCAG | |
| 4 | 40602 | GTATGAGACACCACACCTGCCCATTTTTGTTTGGTTTTTTAATGGGC | 49 |
| | | AG | |
| 5 | 40603 | GTACAAATATATATCAAATTCATAGATATCTATTGGTACCTCATATA | 59 |
| | | AGTACCATAGAG | |
| 6 | 40604 | GTTCCGGAGCCCCGGCGCGGGCGGGTTCTGGGGTGTAGACGCTGCTG | 67 |
| | | GCCAGCCCGCCCCAGCCGAG | |
| 7 | 40605 | GTGTTTGACGGCATCCCACCGCCCTACGACAAGAAAAAGCGGATGGT | 66 |
| | | GGTTCCTGCTGCCCTCAAG | |
| 8 | 40606 | GTCGCCAGGTAGGGCTGGGGGCCGAGGGACCGGCTCGGGGGCGGGGG | 86 |
| | | GGAAGTGTGCCTGACCGGTCTCTGTCCTCAGCGAGGGAG | |
| 9 | 40607 | GTGGGTCCCAGCCCCGCCCGCTGCCCGGCCGCCCCGCAGGTCCCCCG | 67 |
| | | TGACACCGGCTCCTCCTCAG | |
| 10 | 40608 | GTAAGTGCAGAGGCTGGCAGAGGGCAGCCCATGCCCCCACCTGCCAC | 70 |
| | | CTCACAAGCCTCTCCTCCCACAG | |
| 11 | 40609 | GTGAGTCTATGGGACCCTTGATGTTTTCTTTCCCCTTCTTTTCTATG | 476 |
| | | GTTAAGTTCATGTCATAGGAAGGGGAGAAGTAACAGGGTACACATAT | |
| | | TGACCAAATCAGGGTAATTTTGCATTTGTAATTTTAAAAAATGCTTT | |
| | | CTTCTTTTAATATACTTTTTTGTTTATCTTATTTCTAATACTTTCCC | |
| | | TAATCTCTTTCTTTCAGGGCAATAATGATACAATGTATCATGCCTCT | |
| | | TTGCACCATTCTAAAGAATAACAGTGATAATTTCTGGGTTAAGGCAA | |
| | | TAGCAATATTTCTGCATATAAATATTTCTGCATATAAATTGTAACTG | |
| | | ATGTAAGAGGTTTCATATTGCTAATAGCAGCTACAATCCAGCTACCA | |
| | | TTCTGCTTTTATTTTATGGTTGGGATAAGGCTGGATTATTCTGAGTC | |
| | | CAAGCTAGGCCCTTTTGCTAATCATGTTCATACCTCTTATCTTCCTC | |
| | | CCACAG | |
| 12 | 40610 | GTAAGTGGAGACTAGGGGGCTGGGGTTGCACCCTCCCAGTCTGACTC | 69 |
| | | CTCACTGCCGCCGCCTCCTCAG | |
| 13 | 40611 | GTGAGCTGGCGCCCCCAGGGCGGCTCCGGGCCCAGGCCCGTCCAGGG | 69 |
| | | CATAACCCCCTGTCTCCCCTAG | |
| 14 | 40612 | GTAGGCGCCTGGGGGGGGCAGGAGGGTACACGGGCGTAAACTGAGTC | 70 |
| | | TCACCGCTTTCCTCTCCCTGCAG | |
| 15 | 40613 | GTGAGTTGGGACTAGGGGTTGGGTCTGGGTCCAGACCCGGCCCAGCC | 70 |
| | | ATCACACACCTGCCCTCCCTCAG | |
| 16 | 40614 | GTACGATGGCACCTCCGGCAAAGAGAGCCAGGAGAGGTAAGGGTGTG | 108 |
| | | TTAGTAAAGTGGGGGGAGGGGAAAGATTTAATAACTTAACTAAGTAT | |
| | | GTGTTTTTTTATAG | |
| 17 | 40615 | GTGAGCTGCGCGCGCGCGGCGGGGGGCGGGCGCCCGGACCCCGCTGA | 69 |
| | | GGCTGCGCCCCTGTCCCCGCAG | |
| 18 | 40616 | GTTCGAGCTTTTGGAGTACGTCGTCTTTAGGTTGGGGGGAGGGGTTT | 206 |
| | | TATGCGATGGAGTTTCCCCACACTGAGTGGGTGGAGACTGAAGTTAG | |
| | | GCCAGCTTGGCACTTGATGTAATTCTCCTTGGAATTTGCCCTTTTTG | |
| | | AGTTTGGATCTTGGTTCATTCTCAAGCCTCAGACAGTGGTTCAAAGT | |
| | | TTTTTTCTTCCATTTCAG | |
| 19 | 40617 | GTGTGTGCTGGGCAGGGTTGGGGGCTGGGGGCCAGGGCATGCCAGGC | 70 |
| | | TCTGATTGCCACCCCCTTTTTAG | |
| 20 | 40618 | GTAAGGCAGGCTCCCTGGGGCGGCAGGTGGGTTGCATGGAGCCAGGC | 68 |
| | | TGACCCTCCATGTCCCCCCAG | |
| 21 | 40619 | GTTTGTTTCCTTTTTTAAAATACATTGAGTATGCTTGCCTTTTAGAT | 299 |
| | | ATAGAAATATCTGATGCTGTCTACTTCACTAAATTTTGATTACATGA | |
| | | TTTGACAGCAATATTGAAGAGTCTAACAGCCAGCACGCAGGTTGGTA | |
| | | AGTACTGTGGGAACATCACAGATTTTGGCTCCATGCCCTAAAGAGAA | |
| | | ATTGGCTTTCAGATTATTTGGATTAAAAACAAAGACTTTCTTAAGAG | |
| | | ATGTAAAATTTTCATGATGTTTTCTTTTTTGCTAAAACTAAAGAATT | |
| | | ATTCTTTTACATTTCAG | |
| 22 | 40620 | GTCGCTGCGACGCTGCCTTCGCCCCGTGCCCCGCTCCGCCGCCGCCT | 226 |
| | | CGCGCCGCCCGCCCCGGCTCTGACTGACCGCGTTACTCCCACAGGTG | |
| | | AGCGGGCGGGACGGCCCTTCTCCTCCGGGCTGTAATTAGCTGAGCAA | |
| | | GAGGTAAGGGTTTAAGGGATGGTTGGTTGGTGGGGTATTAATGTTTA | |
| | | ATTACCTGGAGCACCTGCCTGAAATCACTTTTTTTCAG | |
| 23 | 40621 | GTAAGAGTCAGAGCTGGCAGGGACGTACAGTGGCCACGACTGGGGTA | 71 |
| | | CTGAGCTGCAGTTCACCTGGCAGA | |
| 24 | 40622 | GTGGGTAGGGTTTGGGGGAGAGCGTGGGCTGGGGTTCAGGGACACCC | 69 |
| | | TCTCACCACTGCCCTCCCACAG | |
| 25 | 40623 | GTAAGTATACAATTGGATGTGCTAAATTGAACAAAATAGGTTCTTGT | 87 |
| | | GCTATTTTACTTAGGTTTCTCTTTTTTTCCCCACACATAG | |
| 26 | 40624 | AGGTAAGGGTTTAAGGGATGGTTGGTTGGTGGGGTATTAATGTTTAA | 84 |
| | | TTACCTGGAGCACCTGCCTGAAATCACTTTTTTTCAG | |
| 27 | 40625 | GTAAGGGTTTAAGGGATGGTTGGTTGGTGGGGTATTAATGTTTAATT | 82 |
| | | ACCTGTTTTACAGGCCTGAAATCACTTGGTTTTAG | |
| 28 | 40626 | GTGAGCCAGGCCGTGGGAGGGCGCCCCCGAGACTGCCACCTGCTCAC | 66 |
| | | CACCCCCCTCTGCTCGTAG | |
| 29 | 40627 | GTGAGTGGGCGCCCCGGCGGGGTGGGCAGTGGGCGGGCCCGAGCTGA | 65 |
| | | CCGCACCCCTCCCCACAG | |
| 30 | 40628 | GTGCGTGAGCGGGGACTGGCGGGGGGTGCCCCCACGGGACCGCGCTG | 66 |
| | | AACCCGGCCCCCCACACAG | |
| 31 | 40629 | GTAGGATGGCGCCTCCTGCAAAAAGAGCAAGAGGTAAGGGTAGTTTT | 106 |
| | | AAGGGGGTGGTGGGCATACATATAAAACTAACTGCAAATAATTTTTT | |
| | | TATATATTACAG | |
| 32 | 40630 | GTAGGCCCTGGCCTGCAGGGACTGTGGGTGCCCCCTGTCCAGTACCC | 69 |
| | | TCACCATGACCCTGTTGCCCAG | |
| 33 | 40631 | GTGAGTCAGGGTGGGGCTGGCCCCCTGCTTCGTGCCCATCCGCGCTC | 68 |
| | | TGACTCTCTGCCCACCTGCAG | |
| 34 | 40632 | GTACTACGGCCTGGGTAGGGAATGGTGGGTGGGGGCGGGGGACCCCT | 68 |
| | | TACCAAGGCCACCCTCTGCAG | |
| 35 | 40633 | GTAAGTTTAGTCTTTTTGTCTTTTATTTCAGGTCCCGGATCCGGTGG | 97 |
| | | TGGTGCAAATCAAAGAACTGCTCCTCAGTGGATGTTGCCTTTACTTC | |
| | | TAG | |
| 36 | 40634 | GTAAGTCACTGACTGTCTATGCCTGGGAAAGGGTGGGCAGGAGATGG | 140 |
| | | GGCAGTGCAGGAAAAGTGGCACTATGAACCCTGCAGCCCTAGGAATG | |
| | | CATCTAGACAATTGTACTAACCTTCTTCTCTTTCCTCTCCTGACAG | |
| 37 | 40635 | GTAAGTATCAAGGTTACAAGACAGGTTTAAGGAGACCAATAGAAACT | 133 |
| | | GGGCTTGTCGAGACAGAGGAGACTCTTGCGTTTCTGATAGGCACCTA | |
| | | TTGGTCTTACTGACATCCACTTTGCCTTTCTCTCCACAG | |
| 38 | 40636 | GTAAATTTCTAGTTTTTCTCCTTCATTTTCTTGGTTAGGACCCTTTT | 190 |
| | | CTCTTTTTATTTTTTTGAGCTTTGATCTTTCTTTAAACTGATCTATT | |
| | | TTTTAATTGATTGGTTATGGTGTAAATATTACATAGCTTTAACTGAT | |
| | | AATCTGATTACTTTATTTCGTGTGTCTATGATGATGATGATAGTTAC | |
| | | AG | |
| 39 | 40637 | GTAAGTACCGCCTATAGAGTCTATAGGCCCACCCCCTTGGCTTCTTA | 271 |
| | | TGCATGCTATACTGTTTTTGGCTTGGGGTCTATACACCCCCGCTTCC | |
| | | TCATGTTATAGGTGATGGTATAGCTTAGCCTATAGGTGTGGGTTATT | |
| | | GACCATTATTGACCACTCCAACGGTGGAGGGCAGTGTAGTCTGAGCA | |
| | | GTACTCGTTGCTGCCGCGCGCGCCACCAGACATAATAGCTGACAGAC | |
| | | TAACAGACTGTTCCTTTCCATGGGTCTTTTCTGCAG | |
| 40 | 40638 | GTAAGTACCGCCTATAGACTCTATAGGCACACCCCTTTGGCTCTTAT | 96 |
| | | GCATGCTGACAGACTAACAGACTGTTCCTTTCCTGGGTCTTTTCTGC | |
| | | AG | |
| 41 | 40639 | GTAAGTACCGCCTATAGACTCTATAGGCACACCCCTTTGGCTCTTAT | 110 |
| | | GCATGAATTAATACGACTCACTATAGGGAGACAGACTGTTCCTTTCC | |
| | | TGGGTCTTTTCTGCAG | |
| 42 | 40640 | GTAAGTACCGCCTATAGACTCTATAGGCACACCCCTTTGGCTCTTAT | 270 |
| | | GCATGCTATACTGTTTTTGGCTTGGGGCCTATACACCCCCGCTTCCT | |
| | | TATGCTATAGGTGATGGTATAGCTTAGCCTATAGGTGTGGGTTATTG | |
| | | ACCATTATTGACCACTCCAACGGTGGAGGGCAGTGTAGTCTGAGCAG | |
| | | TACTCGTTGCTGCCGCGCGCGCCACCAGACATAATAGCTGACAGACT | |
| | | AACAGACTGTTCCTTTCCATGGGTCTTTTCTGCAG | |
| 43 | 40641 | GTAAGTACTTTGCTACATCCATACTCCATCCTTCCCATCCCTTATTC | 116 |
| | | CTTTGAACCTTTCAGTTCGAGCTTTCCCACTTCATCGCAGCTTGACT | |
| | | AACAGCTACCCCGCTTGAGCAG | |
| 44 | 40642 | GTAGGTTCAACCACTGATGCCTAGGCACACCGAAACGACTAACCCTA | 67 |
| | | ATTCTTATCCTTTACTTCAG | |
| 45 | 40643 | GTAAATATAAAATTTTTAAGTGTATAATGTGTTAAACTACTGATTCT | 66 |
| | | AATTGTTTGTGTATTTTAG | |

### Results:

The effects of introns on transgene expression is to be assessed by cloning 50 different introns into AAV-cis plasmid and then assaying for editing in the tdTomato assay.

When compared to the base construct without an intron, the addition of an intronic sequence generally increases the overall editing efficiency of AAV transgenes.

The results are expected to support that the addition of introns to AAV-transgenes expressing CasX under the control of short but strong promoter sequences will enable increased CasX expression and on-target editing while reducing cargo size, further optimizing the AAV system.

### Example 12: Improved Guide Variants Demonstrate Enhanced On-Target Activity In Vitro

Experiments were conducted to identify engineered guide RNA variants with increased activity at different genomic targets, including the therapeutically-relevant mouse and human Rho exon 1. Previous assays identified many different “hotspot” regions (e.g., stem loop) within the scaffold sequences holding the potential to significantly increase editing efficiency as well as specificity. Additionally, screens were conducted to identify scaffold variants that would increase the overall activity of our CRISPR system in an AAV vector across multiple different PAM-spacer combinations, without triggering off-target or non-specific editing. Achieving increased editing efficiency compared to current benchmark vectors would allow reduced viral vector doses to be used in in vivo studies, improving the safety of AAV-mediated CasX-guide systems.

### Methods:

New gRNA scaffold and spacer variants were inserted into an AAV transgene construct for plasmid and viral vector validation (encoding sequences in Tables 18 and 19). CasX 491 variant protein was used for all constructs evaluated in this experiment, however the disclosure contemplates utilizing any of the CasX variants, including those of Table 3 and the encoding sequences of Table 21. We conceptually broke up the AAV transgene between ITRs into different parts, which consisted of our therapeutic cargo and accessory elements relevant to expression in mammalian cells and our nuclease-guide RNA complex (protein nuclease, scaffold, spacer). A schematic and its conceptual parts is shown in FIG. 42. The nucleic acid sequences of the remaining components common to the various constructs are presented in Table 26, the encoding sequences of the guides are presented in Tables 18 and 19, and the encoding sequences of the CasX are presented in Table 21 such that the various permutations of the transgene can be elucidated.

Cloning: Each part in the AAV genome was separated by restriction enzyme sites to allow for modular cloning. Parts were ordered as gene fragments from Twist, PCR amplified, and digested with corresponding restriction enzymes, cleaned, then ligated into a vector also digested with the same enzymes. New AAV constructs were then transformed into chemically competent E. coli (Turbos or Stbl3s). Transformed cells were recovered for 1 hour in a 37° C. shaking incubator then plated on Kanamycin LB-Agar plates and allowed to grow at 37° C. for 12-16 hours. Colonies were picked into 6 mL of 2xyt treated with Kanamycin and allowed to grow for 7-14 hours, then mini-prepped and Sanger sequenced. The transformation and miniprep protocol were then repeated and spacer-cloned vectors were sequence verified again. Validated constructs were maxi-prepped. To assess the quality of maxi-preps, constructs were processed in two separate digests with XmaI (which cuts at several sites in each of the ITRs) and XhoI which cuts once in the AAV genome. These digests and the uncut construct were then run on a 1% Agarose gel and imaged on a ChemiDoc. If the plasmid was >90% supercoiled, the correct size, and the ITRs were intact, the construct moved on to be tested via nucleofection and subsequently used for AAV vector production.

TABLE 18
Guide sequences cloned into p59.491.U6.X.Y. plasmids (X = guide ; Y = spacer)

| | SEQ | | SEQ | | SEQ | |
|---|---|---|---|---|---|---|
| Guide.spacer | ID | Spacer | ID | sgRNA Guide | ID | sgRNA Guide + |
| Construct | NO: | Sequence | NO: | Sequence | NO: | Spacer Sequence |
| 174.11.30 | 40502 | AAGGGGCTC | 40506 | ACTGGCGCTTTTA | 40517 | ACTGGCGCTTTTATC |
| | | CGCACCACG | | TCTGATTACTTTG | | TGATTACTTTGAGAG |
| | | CC | | AGAGCCATCACCA | | CCATCACCAGCGACT |
| | | | | GCGACTATGTCGT | | ATGTCGTAGTGGGTA |
| | | | | AGTGGGTAAAGCT | | AAGCTCCCTCTTCGG |
| | | | | CCCTCTTCGGAGG | | AGGGAGCATCAAAGA |
| | | | | GAGCATCAAAG | | AGGGGCTCCGCACCA |
| | | | | | | CGCC |
| 229.11.30 | 40502 | AAGGGGCTC | 40507 | ACTGGCACTTTTA | 40518 | ACTGGCACTTTTATC |
| | | CGCACCACG | | TCTGATTACTTTG | | TGATTACTTTGAGAG |
| | | CC | | AGAGCCATCACCA | | CCATCACCAGCGACT |
| | | | | GCGACTATGTCGT | | ATGTCGTATGGGTAA |
| | | | | ATGGGTAAAGCGC | | AGCGCTTACGGACTT |
| | | | | TTACGGACTTCGG | | CGGTCCGTAAGAAGC |
| | | | | TCCGTAAGAAGCA | | ATCAAAGAAGGGGCT |
| | | | | TCAAAG | | CCGCACCACGCC |
| 230.11.30 | 40502 | AAGGGGCTC | 40508 | ACTGGCACTTCTA | 40519 | ACTGGCACTTCTATC |
| | | CGCACCACG | | TCTGATTACTCTG | | TGATTACTCTGAGAG |
| | | CC | | AGAGCCATCACCA | | CCATCACCAGCGACT |
| | | | | GCGACTATGTCGT | | ATGTCGTATGGGTAA |
| | | | | ATGGGTAAAGCGC | | AGCGCTTACGGACTT |
| | | | | TTACGGACTTCGG | | CGGTCCGTAAGAAGC |
| | | | | TCCGTAAGAAGCA | | ATCAGAAAGGGGCTC |
| | | | | TCAGA | | CGCACCACGCC |
| 231.11.30 | 40502 | AAGGGGCTC | 40509 | ACTGGCGCTTCTA | 40520 | ACTGGCGCTTCTATC |
| | | CGCACCACG | | TCTGATTACTCTG | | TGATTACTCTGAGAG |
| | | CC | | AGAGCCATCACCA | | CCATCACCAGCGACT |
| | | | | GCGACTATGTCGT | | ATGTCGTATGGGTAA |
| | | | | ATGGGTAAAGCCG | | AGCCGCTTACGGACT |
| | | | | CTTACGGACTTCG | | TCGGTCCGTAAGAGG |
| | | | | GTCCGTAAGAGGC | | CATCAGAGAAGGGGC |
| | | | | ATCAGAG | | TCCGCACCACGCC |
| 232.11.30 | 40502 | AAGGGGCTC | 40510 | ACTGGCACTTCTA | 40521 | ACTGGCACTTCTATC |
| | | CGCACCACG | | TCTGATTACTCTG | | TGATTACTCTGAGCG |
| | | CC | | AGCGCCATCACCA | | CCATCACCAGCGACT |
| | | | | GCGACTATGTCGT | | ATGTCGTATGGGTAA |
| | | | | ATGGGTAAAGCCG | | AGCCGCTTACGGACT |
| | | | | CTTACGGACTTCG | | TCGGTCCGTAAGAGG |
| | | | | GTCCGTAAGAGGC | | CATCAGAGAAGGGGC |
| | | | | ATCAGAG | | TCCGCACCACGCC |
| 233.11.30 | 40502 | AAGGGGCTC | 40511 | ACTGGCGCTTCTA | 40522 | ACTGGCGCTTCTATC |
| | | CGCACCACG | | TCTGATTACTCTG | | TGATTACTCTGAGCG |
| | | CC | | AGCGCCATCACCA | | CCATCACCAGCGACT |
| | | | | GCGACTATGTCGT | | ATGTCGTATGGGTAA |
| | | | | ATGGGTAAAGCCG | | AGCCGCTTACGGACT |
| | | | | CTTACGGACTTCG | | TCGGTCCGTAAGAGG |
| | | | | GTCCGTAAGAGGC | | CATCAGAGAAGGGGC |
| | | | | ATCAGAG | | TCCGCACCACGCC |
| 234.11.30 | 40502 | AAGGGGCTC | 40512 | ACTGGCGCTTCTA | 40523 | ACTGGCGCTTCTATC |
| | | CGCACCACG | | TCTGATTACTCTG | | TGATTACTCTGAGCG |
| | | CC | | AGCGCCATCACCA | | CCATCACCAGCGACT |
| | | | | GCGACTATGTCGT | | ATGTCGTATGGGTAA |
| | | | | ATGGGTAAAGCGC | | AGCGCCTTACGGACT |
| | | | | CTTACGGACTTCG | | TCGGTCCGTAAGGAG |
| | | | | GTCCGTAAGGAGC | | CATCAGAGAAGGGGC |
| | | | | ATCAGAG | | TCCGCACCACGCC |
| 235.11.30 | 40502 | AAGGGGCTC | 40513 | ACTGGCGCTTCTA | 40524 | ACTGGCGCTTCTATC |
| | | CGCACCACG | | TCTGATTACTCTG | | TGATTACTCTGAGCG |
| | | CC | | AGCGCCATCACCA | | CCATCACCAGCGACT |
| | | | | GCGACTATGTCGT | | ATGTCGTAGTGGGTA |
| | | | | AGTGGGTAAAGCC | | AAGCCGCTTACGGAC |
| | | | | GCTTACGGACTTC | | TTCGGTCCGTAAGAG |
| | | | | GGTCCGTAAGAGG | | GCATCAGAGAAGGGG |
| | | | | CATCAGAG | | CTCCGCACCACGCC |
| 236.11.30 | 40502 | AAGGGGCTC | 40514 | ACGGGACTTTCTA | 40525 | ACGGGACTTTCTATC |
| | | CGCACCACG | | TCTGATTACTCTG | | TGATTACTCTGAAGT |
| | | CC | | AAGTCCCTCACCA | | CCCTCACCAGCGACT |
| | | | | GCGACTATGTCGT | | ATGTCGTATGGGTAA |
| | | | | ATGGGTAAAGCCG | | AGCCGCTTACGGACT |
| | | | | CTTACGGACTTCG | | TCGGTCCGTAAGAGG |
| | | | | GTCCGTAAGAGGC | | CATCAGAGAAGGGGC |
| | | | | ATCAGAG | | TCCGCACCACGCC |
| 237.11.30 | 40502 | AAGGGGCTC | 40515 | ACCTGTAGTTCTA | 40526 | ACCTGTAGTTCTATC |
| | | CGCACCACG | | TCTGATTACTCTG | | TGATTACTCTGACTA |
| | | CC | | ACTACAGTCACCA | | CAGTCACCAGCGACT |
| | | | | GCGACTATGTCGT | | ATGTCGTATGGGTAA |
| | | | | ATGGGTAAAGCCG | | AGCCGCTTACGGACT |
| | | | | CTTACGGACTTCG | | TCGGTCCGTAAGAGG |
| | | | | GTCCGTAAGAGGC | | CATCAGAGAAGGGGC |
| | | | | ATCAGAG | | TCCGCACCACGCC |
| 174.11.31 | 40503 | AAGTGGCTC | 40516 | ACTGGCGCTTTTA | 40517 | ACTGGCGCTTTTATC |
| | | CGCACCACG | | TCTGATTACTTTG | | TGATTACTTTGAGAG |
| | | CC | | AGAGCCATCACCA | | CCATCACCAGCGACT |
| | | | | GCGACTATGTCGT | | ATGTCGTAGTGGGTA |
| | | | | AGTGGGTAAAGCT | | AAGCTCCCTCTTCGG |
| | | | | CCCTCTTCGGAGG | | AGGGAGCATCAAAGA |
| | | | | GAGCATCAAAG | | AGGGGCTCCGCACCA |
| | | | | | | CGCC |
| 235.11.31 | 40503 | AAGTGGCTC | 40506 | ACTGGCGCTTCTA | 40527 | ACTGGCGCTTCTATC |
| | | CGCACCACG | | TCTGATTACTCTG | | TGATTACTCTGAGCG |
| | | CC | | AGCGCCATCACCA | | CCATCACCAGCGACT |
| | | | | GCGACTATGTCGT | | ATGTCGTAGTGGGTA |
| | | | | AGTGGGTAAAGCC | | AAGCCGCTTACGGAC |
| | | | | GCTTACGGACTTC | | TTCGGTCCGTAAGAG |
| | | | | GGTCCGTAAGAGG | | GCATCAGAGAAGTGG |
| | | | | CATCAGAG | | CTCCGCACCACGCC |
| 174.11.1 | 40504 | AAGGGGCTG | 40506 | ACTGGCGCTTTTA | 40528 | ACTGGCGCTTTTATC |
| | | CGTACCACA | | TCTGATTACTTTG | | TGATTACTTTGAGAG |
| | | CC | | AGAGCCATCACCA | | CCATCACCAGCGACT |
| | | | | GCGACTATGTCGT | | ATGTCGTAGTGGGTA |
| | | | | AGTGGGTAAAGCT | | AAGCTCCCTCTTCGG |
| | | | | CCCTCTTCGGAGG | | AGGGAGCATCAAAGA |
| | | | | GAGCATCAAAG | | AGGGGCTGCGTACCA |
| | | | | | | CACC |
| 235.11.1 | 40504 | AAGGGGCTG | 40514 | ACTGGCGCTTCTA | 40529 | ACTGGCGCTTCTATC |
| | | CGTACCACA | | TCTGATTACTCTG | | TGATTACTCTGAGCG |
| | | CC | | AGCGCCATCACCA | | CCATCACCAGCGACT |
| | | | | GCGACTATGTCGT | | ATGTCGTAGTGGGTA |
| | | | | AGTGGGTAAAGCC | | AAGCCGCTTACGGAC |
| | | | | GCTTACGGACTTC | | TTCGGTCCGTAAGAG |
| | | | | GGTCCGTAAGAGG | | GCATCAGAGAAGGGG |
| | | | | CATCAGAG | | CTGCGTACCACACC |
| 235.NT | 40505 | GGGTCTTCG | 40506 | ACTGGCGCTTCTA | 40530 | ACTGGCGCTTCTATC |
| | | AGAAGACCC | | TCTGATTACTCTG | | TGATTACTCTGAGCG |
| | | | | AGCGCCATCACCA | | CCATCACCAGCGACT |
| | | | | GCGACTATGTCGT | | ATGTCGTAGTGGGTA |
| | | | | AGTGGGTAAAGCC | | AAGCCGCTTACGGAC |
| | | | | GCTTACGGACTTC | | TTCGGTCCGTAAGAG |
| | | | | GGTCCGTAAGAGG | | GCATCAGAGGGGTCT |
| | | | | CATCAGAG | | TCGAGAAGACCC |

TABLE 19
Guide sequences cloned into p59.491.U6.X.Y. plasmids. (X = guide , Y = spacer) with
spacer length variants

| Guide.spacer | Spacer | SEQ ID | Spacer | SEQ ID | Guide | SEQ ID | |
|---|---|---|---|---|---|---|---|
| Construct | length | NO: | Sequence | NO: | Sequence | NO: | Guide + Spacer Sequence |
| 174.11.30 | 20 nt | 40531 | AAGGGGCT | 40543 | ACTGGCGCT | 40545 | ACTGGCGCTTTTATCTGA |
| | | | CCGCACCA | | TTTATCTGA | | TTACTTTGAGAGCCATCA |
| | | | CGCC | | TTACTTTGA | | CCAGCGACTATGTCGTAG |
| | | | | | GAGCCATCA | | TGGGTAAAGCTCCCTCTT |
| | | | | | CCAGCGACT | | CGGAGGGAGCATCAAAGA |
| | | | | | ATGTCGTAG | | AGGGGCTCCGCACCACGC |
| | | | | | TGGGTAAAG | | C |
| | | | | | CTCCCTCTT | | |
| | | | | | CGGAGGGAG | | |
| | | | | | CATCAAAG | | |
| 174.11.39 | 19 nt | 40532 | AAGGGGCT | 40543 | ACTGGCGCT | 40546 | ACTGGCGCTTTTATCTGA |
| | | | CCGCACCA | | TTTATCTGA | | TTACTTTGAGAGCCATCA |
| | | | CGC | | TTACTTTGA | | CCAGCGACTATGTCGTAG |
| | | | | | GAGCCATCA | | TGGGTAAAGCTCCCTCTT |
| | | | | | CCAGCGACT | | CGGAGGGAGCATCAAAGA |
| | | | | | ATGTCGTAG | | AGGGGCTCCGCACCACGC |
| | | | | | TGGGTAAAG | | |
| | | | | | CTCCCTCTT | | |
| | | | | | CGGAGGGAG | | |
| | | | | | CATCAAAG | | |
| 174.11.38 | 18 nt | 40533 | AAGGGGCT | 40543 | ACTGGCGCT | 40547 | ACTGGCGCTTTTATCTGA |
| | | | CCGCACCA | | TTTATCTGA | | TTACTTTGAGAGCCATCA |
| | | | CG | | TTACTTTGA | | CCAGCGACTATGTCGTAG |
| | | | | | GAGCCATCA | | TGGGTAAAGCTCCCTCTT |
| | | | | | CCAGCGACT | | CGGAGGGAGCATCAAAGA |
| | | | | | ATGTCGTAG | | AGGGGCTCCGCACCACG |
| | | | | | TGGGTAAAG | | |
| | | | | | CTCCCTCTT | | |
| | | | | | CGGAGGGAG | | |
| | | | | | CATCAAAG | | |
| 174.11.31 | 20 nt | 40534 | AAGTGGCT | 40543 | ACTGGCGCT | 40548 | ACTGGCGCTTTTATCTGA |
| | | | CCGCACCA | | TTTATCTGA | | TTACTTTGAGAGCCATCA |
| | | | CGCC | | TTACTTTGA | | CCAGCGACTATGTCGTAG |
| | | | | | GAGCCATCA | | TGGGTAAAGCTCCCTCTT |
| | | | | | CCAGCGACT | | CGGAGGGAGCATCAAAGA |
| | | | | | ATGTCGTAG | | AGTGGCTCCGCACCACGC |
| | | | | | TGGGTAAAG | | C |
| | | | | | CTCCCTCTT | | |
| | | | | | CGGAGGGAG | | |
| | | | | | CATCAAAG | | |
| 174.11.37 | 19 nt | 40535 | AAGTGGCT | 40543 | ACTGGCGCT | 40549 | ACTGGCGCTTTTATCTGA |
| | | | CCGCACCA | | TTTATCTGA | | TTACTTTGAGAGCCATCA |
| | | | CGC | | TTACTTTGA | | CCAGCGACTATGTCGTAG |
| | | | | | GAGCCATCA | | TGGGTAAAGCTCCCTCTT |
| | | | | | CCAGCGACT | | CGGAGGGAGCATCAAAGA |
| | | | | | ATGTCGTAG | | AGTGGCTCCGCACCACGC |
| | | | | | TGGGTAAAG | | |
| | | | | | CTCCCTCTT | | |
| | | | | | CGGAGGGAG | | |
| | | | | | CATCAAAG | | |
| 174.11.36 | 18 nt | 40536 | AAGTGGCT | 40543 | ACTGGCGCT | 40550 | ACTGGCGCTTTTATCTGA |
| | | | CCGCACCA | | TTTATCTGA | | TTACTTTGAGAGCCATCA |
| | | | CG | | TTACTTTGA | | CCAGCGACTATGTCGTAG |
| | | | | | GAGCCATCA | | TGGGTAAAGCTCCCTCTT |
| | | | | | CCAGCGACT | | CGGAGGGAGCATCAAAGA |
| | | | | | ATGTCGTAG | | AGTGGCTCCGCACCACG |
| | | | | | TGGGTAAAG | | |
| | | | | | CTCCCTCTT | | |
| | | | | | CGGAGGGAG | | |
| | | | | | CATCAAAG | | |
| 235.11.1 | 20 nt | 40537 | AAGGGGCT | 40544 | ACTGGCGCT | 40551 | ACTGGCGCTTCTATCTGA |
| | | | GCGTACCA | | TCTATCTGA | | TTACTCTGAGCGCCATCA |
| | | | CACC | | TTACTCTGA | | CCAGCGACTATGTCGTAG |
| | | | | | GCGCCATCA | | TGGGTAAAGCCGCTTACG |
| | | | | | CCAGCGACT | | GACTTCGGTCCGTAAGAG |
| | | | | | ATGTCGTAG | | GCATCAGAGAAGGGGCTG |
| | | | | | TGGGTAAAG | | CGTACCACACC |
| | | | | | CCGCTTACG | | |
| | | | | | GACTTCGGT | | |
| | | | | | CCGTAAGAG | | |
| | | | | | GCATCAGAG | | |
| 235.11.41 | 19 nt | 40538 | AAGGGGCT | 40544 | ACTGGCGCT | 40552 | ACTGGCGCTTCTATCTGA |
| | | | GCGTACCA | | TCTATCTGA | | TTACTCTGAGCGCCATCA |
| | | | CAC | | TTACTCTGA | | CCAGCGACTATGTCGTAG |
| | | | | | GCGCCATCA | | TGGGTAAAGCCGCTTACG |
| | | | | | CCAGCGACT | | GACTTCGGTCCGTAAGAG |
| | | | | | ATGTCGTAG | | GCATCAGAGAAGGGGCTG |
| | | | | | TGGGTAAAG | | CGTACCACAC |
| | | | | | CCGCTTACG | | |
| | | | | | GACTTCGGT | | |
| | | | | | CCGTAAGAG | | |
| | | | | | GCATCAGAG | | |
| 235.11.40 | 18 nt | 40539 | AAGGGGCT | 40544 | ACTGGCGCT | 40553 | ACTGGCGCTTCTATCTGA |
| | | | GCGTACCA | | TCTATCTGA | | TTACTCTGAGCGCCATCA |
| | | | CA | | TTACTCTGA | | CCAGCGACTATGTCGTAG |
| | | | | | GCGCCATCA | | TGGGTAAAGCCGCTTACG |
| | | | | | CCAGCGACT | | GACTTCGGTCCGTAAGAG |
| | | | | | ATGTCGTAG | | GCATCAGAGAAGGGGCTG |
| | | | | | TGGGTAAAG | | CGTACCACA |
| | | | | | CCGCTTACG | | |
| | | | | | GACTTCGGT | | |
| | | | | | CCGTAAGAG | | |
| | | | | | GCATCAGAG | | |
| 235.11.2 | 20 nt | 40540 | AAGTGGCT | 40544 | ACTGGCGCT | 40554 | ACTGGCGCTTCTATCTGA |
| | | | GCGTACCA | | TCTATCTGA | | TTACTCTGAGCGCCATCA |
| | | | CACC | | TTACTCTGA | | CCAGCGACTATGTCGTAG |
| | | | | | GCGCCATCA | | TGGGTAAAGCCGCTTACG |
| | | | | | CCAGCGACT | | GACTTCGGTCCGTAAGAG |
| | | | | | ATGTCGTAG | | GCATCAGAGAAGTGGCTG |
| | | | | | TGGGTAAAG | | CGTACCACACC |
| | | | | | CCGCTTACG | | |
| | | | | | GACTTCGGT | | |
| | | | | | CCGTAAGAG | | |
| | | | | | GCATCAGAG | | |
| 235.11.43 | 19 nt | 40541 | AAGTGGCT | 40544 | ACTGGCGCT | 40555 | ACTGGCGCTTCTATCTGA |
| | | | GCGTACCA | | TCTATCTGA | | TTACTCTGAGCGCCATCA |
| | | | CAC | | TTACTCTGA | | CCAGCGACTATGTCGTAG |
| | | | | | GCGCCATCA | | TGGGTAAAGCCGCTTACG |
| | | | | | CCAGCGACT | | GACTTCGGTCCGTAAGAG |
| | | | | | ATGTCGTAG | | GCATCAGAGAAGTGGCTG |
| | | | | | TGGGTAAAG | | CGTACCACAC |
| | | | | | CCGCTTACG | | |
| | | | | | GACTTCGGT | | |
| | | | | | CCGTAAGAG | | |
| | | | | | GCATCAGAG | | |
| 235.11.42 | 18 nt | 40542 | AAGTGGCT | 40544 | ACTGGCGCT | 40556 | ACTGGCGCTTCTATCTGA |
| | | | GCGTACCA | | TCTATCTGA | | TTACTCTGAGCGCCATCA |
| | | | CA | | TTACTCTGA | | CCAGCGACTATGTCGTAG |
| | | | | | GCGCCATCA | | TGGGTAAAGCCGCTTACG |
| | | | | | CCAGCGACT | | GACTTCGGTCCGTAAGAG |
| | | | | | ATGTCGTAG | | GCATCAGAGAAGTGGCTG |
| | | | | | TGGGTAAAG | | CGTACCACA |
| | | | | | CCGCTTACG | | |
| | | | | | GACTTCGGT | | |
| | | | | | CCGTAAGAG | | |
| | | | | | GCATCAGAG | | |

TABLE 20
Sequences of AAV vector components common to the plasmids

| | Part | SEQ ID | |
|---|---|---|---|
| Component | Name | NO: | Nucleic Acid Sequence |
| 5′ITR | | 40557 | CCTGCAGGCAGCTGCGCGCTCGCTCGCTCACTGAGGCCGCCCGGGCGTCGGGCGAC |
| | | | CTTTGGTCGCCCGGCCTCAGTGAGCGAGCGAGCGCGCAGAGAGGGAGTGGCCAACT |
| | | | CCATCACTAGGGGTTCCT |
| buffer seq | | 40558 | GCGGCCTCTAGACTCGAGGCGTT |
| enhancer | CMV | 140559 | GACATTGATTATTGACTAGTTATTAATAGTAATCAATTACGGGGTCATTAGTTCAT |
| | | | AGCCCATATATGGAGTTCCGCGTTACATAACTTACGGTAAATGGCCCGCCTGGCTG |
| | | | ACCGCCCAACGACCCCCGCCCATTGACGTCAATAATGACGTATGTTCCCATAGTAA |
| | | | CGCCAATAGGGACTTTCCATTGACGTCAATGGGTGGAGTATTTACGGTAAACTGCC |
| | | | CACTTGGCAGTACATCAAGTGTATCATATGCCAAGTACGCCCCCTATTGACGTCAA |
| | | | TGACGGTAAATGGCCCGCCTGGCATTATGCCCAGTACATGACCTTATGGGACTTTC |
| | | | CTACTTGGCAGTACATCTACGTATTAGTCATCGCTATTACCATG |
| Pol II | CMV | 40435 | GTGATGCGGTTTTGGCAGTACATCAATGGGCGTGGATAGCGGTTTGACTCACGGGG |
| promoter | | | ATTTCCAAGTCTCCACCCCATTGACGTCAATGGGAGTTTGTTTTGGCACCAAAATC |
| | | | AACGGGACTTTCCAAAATGTCGTAACAACTCCGCCCCATTGACGCAAATGGGCGGT |
| | | | AGGCGTGTACGGTGGGAGGTCTATATAAGCAGAGCT |
| buffer seq | | 40561 | CTCTGGCTAACTACC |
| kozac | | 40562 | GGTGCCACCATG |
| start codon | MA | 40563 | ATGGCC |
| 5′NLS | SV40 | 40564 | CCAAAGAAGAAGCGGAAGGTC |
| 5′linker | SR | 40565 | TCTAGA |
| 3′NLS | GS | 40566 | GGATCC |
| linker | | | |
| 3′NLS | SV40 | 249 | CCAAAAAAGAAGAGAAAGGTA |
| tag | HA | 40568 | TACCCATATGATGTCCCTGACTACGCT |
| linker | GS | 40569 | GGATCCTAA |
| buffer seq | | 40570 | GAATTCCTAGAGCTCGCTGATCAGCCTCGA |
| Poly(A) | BgH | 40571 | CTGTGCCTTCTAGTTGCCAGCCATCTGTTGTTTGCCCCTCCCCCGTGCCTTCCTTG |
| | polyA | | ACCCTGGAAGGTGCCACTCCCACTGTCCTTTCCTAATAAAATGAGGAAATTGCATC |
| | | | GCATTGTCTGAGTAGGTGTCATTCTATTCTGGGGGGTGGGGTGGGGCAGGACAGCA |
| | | | AGGGGGAGGATTGGGAAGAGAATAGCAGGCATGCTGGGGA |
| buffer seq | | 40572 | GGTACCGT |
| Pol III | U6 | 40573 | GAGGGCCTATTTCCCATGATTCCTTCATATTTGCATATACGATACAAGGCTGTTAG |
| promoter | promoter | | AGAGATAATTGGAATTAATTTGACTGTAAACACAAAGATATTAGTACAAAATACGT |
| | | | GACGTAGAAAGTAATAATTTCTTGGGTAGTTTGCAGTTTTAAAATTATGTTTTAAA |
| | | | ATGGACTATCATATGCTTACCGTAACTTGAAAGTATTTCGATTTCTTGGCTTTATA |
| | | | TATCTTGTGGAAAGGAC |
| buffer | | 40574 | GAAACACC |
| buffer | | 40575 | TTTTTTTTGGCGGCCGC |
| 3′ITR | | 40576 | AGGAACCCCTAGTGATGGAGTTGGCCACTCCCTCTCTGCGCGCTCGCTCGCTCACT |
| | | | GAGGCCGGGCGACCAAAGGTCGCCCGACGCCCGGGCTTTGCCCGGGCGGCCTCAGT |
| | | | GAGCGAGCGAGCGCGCAGCTGCCTGCAGG |

TABLE 21
Sequence of CasX utilized in AAV

| CasX | SEQ ID NO: | Nucleic Acid Sequence |
|---|---|---|
| 438 | 40577 | ND |
| 491 | 40578 | ND |
| 527 | 40579 | ND |
| 535 | 40580 | ND |
| 536 | 40581 | ND |
| 537 | 40582 | ND |
| 583 | 40583 | ND |
| 668 | 40584 | ND |
| 672 | 40585 | ND |
| 669 | 40586 | ND |
| 670 | 40587 | ND |
| 676 | 40588 | ND |

* ND = no description sequence provided in sequence listing.

Reporter cell lines: A neural progenitor cell line isolated from the Ai9-tdTomato was cultured in suspension in pre-equilibrated mNPC medium (DMEM/F12 with GlutaMax, 10 mM HEPES, 1×MEM Non-Essential Amino Acids, 1× penicillin/streptomycin, 1:1000 2-mercaptoethanol, 1× B-27 supplement, minus vitamin A, 1× N2 with supplemented growth factors bFGF and EGF). Prior to testing, cells were dissociated using accutase, with gentle resuspension, monitoring for complete separation of the neurospheres. Cells were then quenched with media, spun down and resuspended in fresh media. Cells were counted and directly used for nucleofection or 10,000 cells were plated in a 96-well plate coated with PLF (1× Poly-DL-ornithine hydrobromide, 10 mg/mL in sterile diH20, 1× Laminin, and 1× Fibronectin), 2 days prior to AAV transduction.

A HEK293T dual reporter cell line was generated by knocking into HEK293T cells two transgene cassettes that constitutively expressed exon 1 of the human RHO gene linked to GFP and exon 1 of the human P23H.RHO gene linked to mscarlet. The modified cells were expanded by serial passage every 3-5 days and maintained in Fibroblast (FR) medium, consisting of Dulbecco's Modified Eagle Medium (DMEM; Corning Cellgro, #10-013-CV) supplemented with 10% fetal bovine serum (FBS; Seradigm, #1500-500), and 100 Units/mL penicillin and 100 mg/mL streptomycin (100×-Pen-Strep; GIBCO #15140-122), and can additionally include sodium pyruvate (100×, Thermofisher #11360070), non-essential amino acids (100× ThermoFisher #11140050), HEPES buffer (100× ThermoFisher #15630080), and 2-mercaptoethanol (1000× ThermoFisher #21985023). The cells were incubated at 37° C. and 5% CO2. After 1-2 weeks, GFP+/mscarlet+ cells were bulk sorted into FB medium. The reporter lines were expanded by serial passage every 3-5 days and maintained in FB medium in an incubator at 37° C. and 5% C02. Reporter clones were generated by a limiting dilution method. The clonal lines were characterized via flow cytometry, genomic sequencing, and functional modification of the RHO locus using a previously validated RHO targeting CasX molecule. The optimal reporter lines were identified as ones that: i) had a single copies of WTRHO.GFP and mutRHO.mscarlet correctly integrated per cell; ii) maintained doubling times equivalent to unmodified cells; and iii) resulted in reduction in GFP and mscarlet fluorescence upon disruption of the RHO gene when assayed using the methods described below.

Plasmid nucleofection: AAV cis-plasmids driving expression of the CasX-scaffold-guide system were nucleofected in mNPCs using the Lonza P3 Primary Cell 96-well Nucleofector Kit. For the ARPE-19 line, the Lonza SF solution and supplement was used. Plasmids were diluted to concentrations of 200 ng/p, 100 ng/μL. 5 μL of DNA per construct was added to the P3 or SF solution containing 200,000 tdTomato mNPCs or ARPE-19 cells respectively. The combined solution was nucleofected using a Lonza 4D Nucleofector System according to manufacturer's guidelines. Following nucleofection, the solution was quenched with appropriate culture media. The solution was then aliquoted in triplicate (approx. 67,000 cells per well) in a 96-well plate. 48 hours after transfection, treated mNPCs were replenished with fresh mNPC media containing growth factors and treated ARPE-19 cells were replenished with fresh FB medium. 5 days after transfection, tdTomato mNPCs and ARPE-19 cells were lifted and activity was assessed by FACS.

AAV vectors production: Suspension HEK293T cells were adapted from parental HEK293T and grown in FreeStyle 293 media. For screening purposes, small scale cultures (20-30 mL cultured in 125 mL Erlenmeyer flasks and agitated at 110 rpm) were diluted to a density of 1.5e+6 cells/mL on the day of transfection. Endotoxin-free pAAV plasmids with the transgene flanked by ITR repeats were co-transfected with plasmids supplying the adenoviral helper genes for replication and AAV rep/cap genome using PEIMax (Polysciences) in serum-free OPTIMEM media. Cultures were supplemented with 10% CDM4HEK293 (HyClone) 3 hours post-transfection. Three days later, cultures were centrifuged at 1000 rpm for 10 minutes to separate the supernatant from the cell pellet. The supernatant was mixed with 40% PEG 2.5M NaCl (8% final concentration) and incubated on ice for at least 2 hours to precipitate AAV viral particles. The cell pellet, containing the majority of the AAV vectors, was resuspended in lysis media (0.15M NaCl, 50 mM Tris HCl, 0.05% Tween, pH 8.5), sonicated on ice (15 seconds, 30% amplitude) and treated with Benzonase (250 U/μL, Novagen) for 30 minutes at 37° C. Crude lysate and PEG-treated supernatant were then spin at 4000 rpm for 20 minutes at 4° C. to resuspend the PEG precipitated AAV (pellet) with cell debris-free crude lysate (supernatant) clarified further using a 0.45 μM filter.

To determine the viral genome titer, 1 μL from crude lysate viruses was digested with DNase and ProtK, followed by quantitative PCR. 5 μL of digested virus was used in a 25 μL qPCR reaction composed of IDT primetime master mix and a set of primer and 6′FAM/Zen/IBFQ probe (IDT) designed to amplify the CMV promoter region (Fwd 5′-CATCTACGTATTAGTCATCGCTATTACCA-3′ (SEQ ID NO: 40801); Rev 5′-GAAATCCCCGTGAGTCAAACC-3′ (SEQ ID NO: 40802), Probe 5′-TCAATGGGCGTGGATAG-3′ (SEQ ID NO: 40803) or a 62 bp-fragment located in the AAV2-ITR (Fwd 5′-GGAACCCCTAGTGATGGAGTT-3′ (SEQ ID NO: 40804); Rev 5′-CGGCCTCAGTGAGCGA-3′ (SEQ ID NO: 40805), Probe 5′-CACTCCCTCTCTGCGCGCTCG-3′ (SEQ ID NO: 40806). Ten-fold serial dilutions (5 μl each of 2e+9 to 2e+4 DNA copies/mL) of an AAV ITR plasmid was used as reference standards to calculate the titer (viral genome (vg)/mL) of viral samples. QPCR program was set up as an initial denaturation step at 95′C for 5 minutes, followed by 40 cycles of denaturation at 95′C for 1 min, and annealing/extension at 60° C. for 1 min.

AAV transduction: 10,000 cells/well of mNPCs were seeded on PLF-coated wells in 96-well plates 48-hours before AAV transduction. All viral infection conditions were performed in triplicate, with normalized number of vg among experimental vectors, in a series of 3-fold dilution of multiplicity of infection (MOI) ranging from ˜1.0e+6 to 1.0e+4 vg/cell. Calculations were based on an estimated number of 20,000 cells per well at the time of transfection. Final volume of 50 μL of AAV vectors diluted in pre-equilibrated mNPC medium supplemented with bFGF/EGF growth factors (20 ng/ml final concentration) were applied to each well. 48 hours post-transfection, complete media change was performed with fresh media supplemented with growth factors. Editing activity (tdT+ cell quantification) was assessed by FACS 5 days post-transfection.

Assessing editing activity by FACS: 5 days after transfection, treated tdTomato mNPCs or ARPE-19 cells in 96-well plates were washed with dPBS and treated with 50 μL TrypLE and Trypsin (0.25%) for 15 and 5 minutes respectively. Following cell dissociation, treated wells were quenched with media containing DMEM, 10% FBS and 1× penicillin/streptomycin. Resuspended cells were transferred to round-bottom 96-well plates and centrifuged for 5 min at 1000× g. Cell pellets were then resuspended with dPBS containing 1×DAPI, and plates were loaded into an Attune NxT Flow Cytometer Autosampler. The Attune NxT flow cytometer was run using the following gating parameters: FSC-A×SSC-A to select cells, FSC-H×FSC-A to select single cells, FSC-A×VL1-A to select DAPI-negative alive cells, and FSC-A×YL1-A to select tdTomato positive cells.

NGS analysis of indels at mRHO exon 1 locus: 5 days after transfection, treated tdTomato mNPCs in 96-well plates were washed with dPBS and treated with 50 μL TrypLE and trypsin (0.25%) for 15 and 5 minutes respectively. Following cell dissociation, treated wells were quenched with media containing DMEM, 10% FBS and 1× penicillin/streptomycin. Cells were then spun down and resulting cell pellets washed with PBS prior to processing them for gDNA extraction using the Zymo mini DNA kit according to the manufacturer's instructions. For assessing editing levels occurring at the mouse RHO exon 1 locus, amplicons were amplified from 200 ng of gDNA with a set of primers (Fwd 5′-ACACTCTTTCCCTACACGACGCTCTTCCGATCTNNNNNNNGCAGCCTTGGTCTCTGT CTACG-3′ (SEQ ID NO: 40595); Rev 5′-GTGACTGGAGTTCAGACGTGTGCTCTTCCGATCTGCCCCAGTCTCTCTGCTCATACC-3′ (SEQ ID NO: 40596), bead-purified (Beckman coulter, Agencourt Ampure XP) and then re-amplified to incorporate illumina adapter sequence. Specifically, these primers contained an additional sequence at the 5′ ends to introduce Illumina read and 2 sequences as well as a 16 nt random sequence that functions as a unique molecular identifier (UMI). Quality and quantification of the amplicon was assessed using a Fragment Analyzer DNA analyzer kit (Agilent, dsDNA 35-1500 bp). Amplicons were sequenced on the Illumina Miseq according to the manufacturer's instructions. Raw fastq files from sequencing were processed as follows: (1) the sequences were trimmed for quality and for adapter sequences using the program cutadapt (v. 2.1); (2) the sequences from read 1 and read 2 were merged into a single insert sequence using the program flash2 (v2.2.00); and (3) the consensus insert sequences were run through the program CRISPResso2 (v 2.0.29), along with the expected amplicon sequence and the spacer sequence. This program quantifies the percent of reads that were modified in a window around the 3′ end of the spacer (30 bp window centered at −3 bp from 3′ end of spacer). The activity of the CasX molecule was quantified as the total percent of reads that contain insertions, substitutions and/or deletions anywhere within this window.

### Results

Different editing experiments were conducted to quantify on-target cleavage mediated by CasX 491 paired with gRNA scaffold variants (guides 174 & 229-237) with different spacers targeting multiple genomic loci of interest. Constructs were cloned into the AAV backbone p59, flanked by ITR2 sequences, driving expression of the protein Cas 491 under the control of a CMV promoter, as well as the scaffold-spacer under the control of the human U6 promoter.

The mNPC-tdT reporter cell line was used to assess single-cut efficiency at the endogenous mouse RHO exon 1 locus (spacer 11.30, CTC PAM). A dual reporter system integrated in a ARPE-19 derived cell line was also used to assess on-target editing at the exogenously expressed human WT Rho locus (spacer 11.1, CTC PAM).

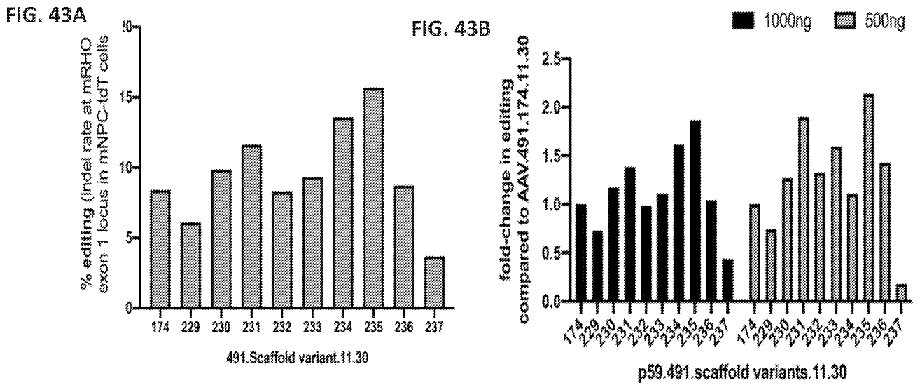

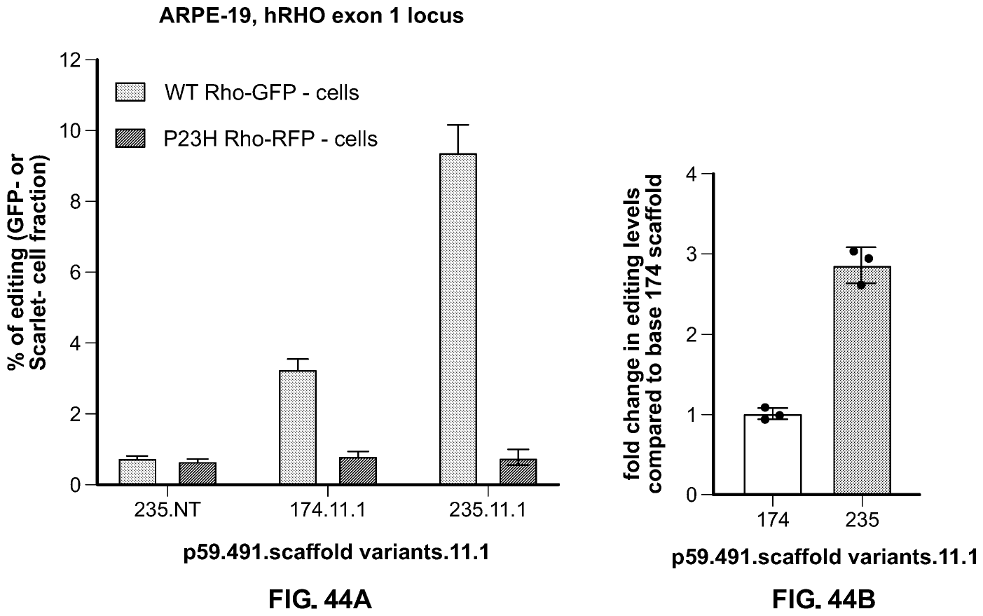

Scaffold variants with spacer 11.30 were tested via nucleofection in the mouse NPC cell line at two different doses, 1000 ng and 500 ng. Constructs were compared to the current benchmark gRNA scaffold 174 activity. Constructs expressing scaffold variants 231, 233, 234, 235 performed at higher levels than ones with scaffold 174.11.30 (FIGS. 43A and 43B). Scaffold 235 displayed a 2-fold increased activity at mRHO exon 1 locus compared to gRNA scaffold 174. We further validated that scaffold 235 consistently improved activity without increased off-target cleavage by nucleofecting the dual reporter ARPE-19 cell line with construct p590.4910.1740.11.1 and p590.4910.2350.11.1, as well as a non-target spacer control. Spacer 11.1 was targeting the exogenously expressed hRHO-GFP gene. Scaffold 235 displayed 3-fold increased activity compared to 174 (9% vs 3% of Rho-GFP− cells respectively, FIGS. 44A and 44B). Allele-specificity was assessed by looking at the frequency of hP23H-RHO-Scarlett-cell population, whose sequence differs from the wild-type by 1 bp.

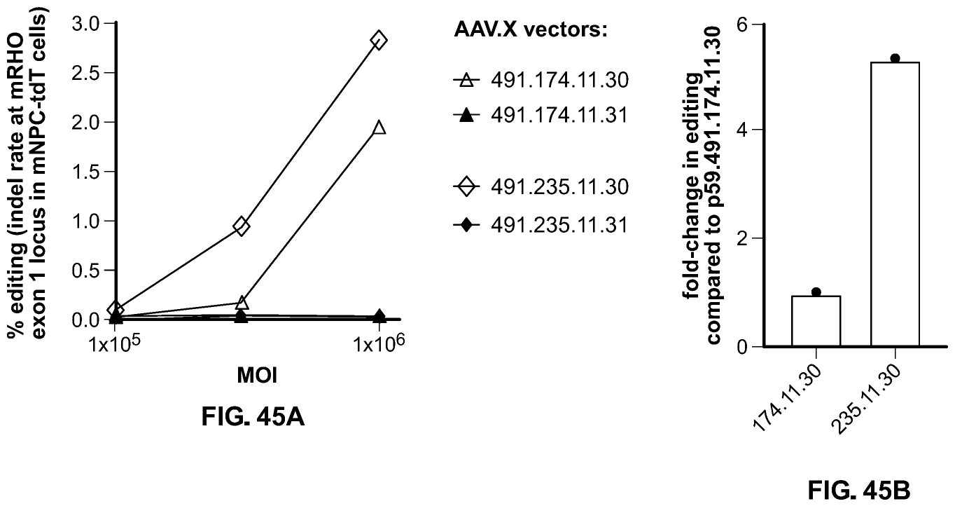

We also sought to demonstrate that these scaffold variants packaged efficiently in AAV and remained potent when delivered virally. mNPC transduced with AAV vectors expressing guide scaffold 235 with spacer 11.30 (on-target, mouse WT RHO) showed increased activity at the on-target locus (>5-fold increase, FIGS. 45A and 45B) compared to ones infected with AAV.491.174.11.30 at 3.0e+5 MOI, with significant no off-target indels detectable with both AAV.491.174.11.31 and AAV.491.235.11.31 vectors targeting the P23H-RHO SNP, respectively.

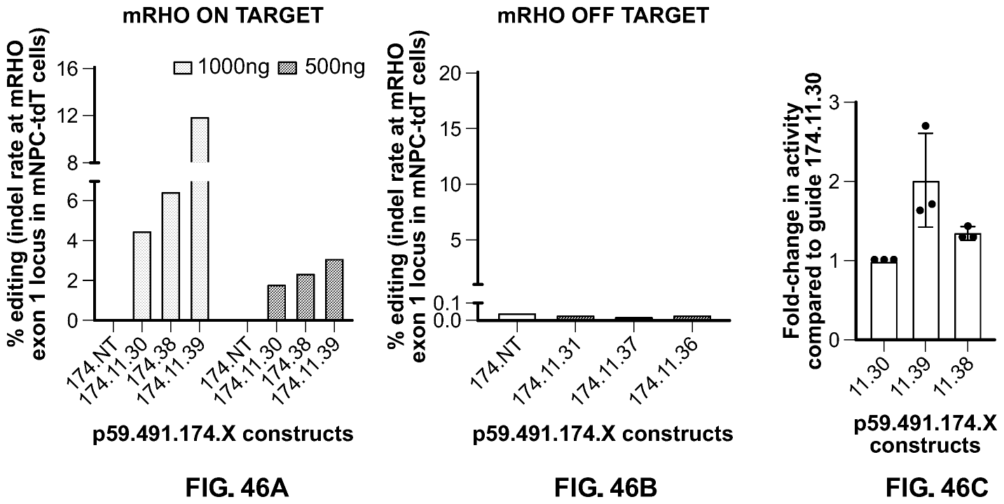

Assessing effects of spacer length: Another set of experiments was conducted to test whether spacer length variants could improve on-target activity. Spacers 11.39, 11.38 and spacer 11.37 (19 nt P23H RHO), 11.36 (18 nt P23H RHO) were designed from parental spacer 11.30 (20 nt WT RHO) and 11.31 (20 nt P23H RHO), respectively, harboring 1 or 2 bp truncations on the 3′ end of the sequence. mfNPC-tdT cells were nucleofected with 1000 ng and 500 ng of constructs p590.4910.1740.11.30 (20 nt WT RHO), p590.4910.1740.11.39 (19 nt WT RHO), p49.491.174.11.38 (18 nt WT RHO), and editing levels were assessed 5 days later. All truncated spacer versions improved editing levels (FIGS. 46A and 46C), with highest improvement observed with p59.491.11.39 constructs (˜2-fold improvement achieved with the 19 bp spacer relative to the 20 bp spacer length construct). No increase in off-target cleavage was observed with truncation spacer variants of the 11.31 spacer targeting the mouse P23H-RHO locus (FIG. 46B).

These results support that scaffold variants with structural mutations can be engineered with increased activity in dual reporter systems investigating therapeutically relevant genomic targets such as the mouse and human RHO exon 1 loci. Furthermore, while the newly characterized scaffold displayed overall >2-fold increase in activity, no off-target cleavage with a 1-bp mismatch spacer region was detected. This is relevant for allele-specific therapeutic strategy such as adRP P23H Rho, which mutated allele differs from WT sequence by 1 nucleotide, targeted by spacer 11.31. This study further validates the use of guide scaffold 235 in AAV vectors designed for P23H RHO rescue and genotoxic studies as well as for other therapeutic targets.

### Example 13: Improved Scaffold and Guide Variants Demonstrate Enhanced On-Target Activity In Vivo

Experiments were conducted to demonstrate that engineered CasX & sgRNA-guide and spacer variants harboring structural mutations that improve selectivity and on-target activity lead to increase edits when delivered in vivo to photoreceptors in the mouse retina, with a spacer targeting the P23 residue at a therapeutically relevant level in the WT. Here, we assessed whether vector expressing CasX variant 491, guide variant 235 and spacer 11.39 improves editing levels compared to parental CasX 491, guide variant 174 and spacer 11.30 in vivo.

### Materials and Methods:

Generation of AAV Plasmids and Viral Vectors: The CasX variant 491 under the control of the RHO promoter, and sgRNA.guide variant 174 with spacer 11.30 and spacer 11.31 (AAGTGGCTCCGCACCACGCC (SEQ ID NO: 40503)) or sgRNA-guide variant 235 with spacer 11.39 (AAGGGGCTCCGCACCACGCC (SEQ ID NO: 40531)) and 11.37 (AAGTGGCTCCGCACCACGC (SEQ ID NO: 40535)) targeting mouse RHO exon 1 at P23 residues) under the U6 promoter were cloned into the p59 plasmid flanked with AAV2 ITR.

Cloning: Each part in the AAV genome was separated by restriction enzyme sites to allow for modular cloning. Parts were ordered as gene fragments from Twist, PCR amplified, and digested with corresponding restriction enzymes, cleaned, then ligated into a vector also digested with the same enzymes. Cas X variant 491 under the RHO promoter and scaffold variants 174 and 235, under the control of the human U6 promoter, were cloned into an AAV backbone, flanked by AAV2 ITRs. Spacers 11.30, 11.31 and variants 11.39, 11.37 were cloned respectively into pAAV.RHO.491.174 and pAAV.RHO.491.235 using Golden Gate cloning. New AAV constructs were then transformed into chemically competent E. coli (Stbl3s). Validated constructs were maxi-prepped. To assess the quality of maxi-preps, constructs were processed in two separate digests with XmaI (which cuts at several sites in each of the ITRs) and XhoI which cuts once in the AAV genome. If the plasmid was >90% supercoiled, the correct size, and the ITRs were intact, the construct was subsequently used for AAV vector production.

AAV vectors production: Suspension HEK293T cells were adapted from parental HEK293T and grown in FreeStyle 293 media. 500 mL cultures (1 L Erlenmeyer flasks, agitated at 110 rpm) were diluted to a density of 2e+6 cells/mL on the day of transfection. Endotoxin-free pAAV plasmids with the transgene flanked by ITR repeats were co-transfected with plasmids supplying the adenoviral helper genes for replication and AAV rep/cap genome using PEIMax (Polysciences) in serum-free OPTIMEM media. Cultures were supplemented with 10% CDM4HEK293 (HyClone) 3 hours post-transfection. Three days later, cultures were centrifuged at 1000 rpm for 10 minutes to separate the supernatant from the cell pellet. The supernatant was mixed with 40% PEG 2.5M NaCl (8% final concentration) and incubated on ice for at least 2 hours to precipitate AAV viral particles. The cell pellet, containing the majority of the AAV vectors, was resuspended in lysis media (0.15 M NaCl, 50 mM Tris HCl, 0.05% Tween, pH 8.5), sonicated on ice (15 seconds, 30% amplitude) and treated with Benzonase (250 U/μL, Novagen) for 30 minutes at 37° C. Crude lysate and PEG-treated supernatant were then spin at 4000 rpm for 20 minutes at 4° C. to resuspend the PEG precipitated AAV (pellet) with cell debris-free crude lysate (supernatant) clarified further using a 0.45 μM filter. AAV lysates were purified using affinity chromatography (POROS CaptureSelect AAVX, ThermoFisher). Eluate was buffer exchanged and concentrated in PBS+200 mM NaCl+0.001% Pluronic.

To determine the viral genome titer, 1 μL from crude lysate viruses was digested with DNase and ProtK, followed by quantitative PCR. 5 μL of digested virus was used in a 25 μL qPCR reaction composed of IDT primetime master mix and a set of primer and 6′FAM/Zen/IBFQ probe (IDT) designed to amplify a 62 bp-fragment located in the AAV2-ITR (Fwd 5′-GGAACCCCTAGTGATGGAGTT-3′ (SEQ ID NO: 40804); Rev 5′-CGGCCTCAGTGAGCGA-3′ (SEQ ID NO: 40805), Probe 5′-CACTCCCTCTCTGCGCGCTCG-3′ (SEQ ID NO: 40806)). An AAV ITR plasmid was used as reference standards to calculate the titer (viral genome (vg)/mL) of viral samples. QPCR program was set up as: initial denaturation step at 95′C for 5 minutes, followed by 40 cycles of denaturation at 95′C for 1 min, and annealing/extension at 60° C. for 1 min.

Subretinal injections C57BL6J mice were obtained from the Jackson Laboratories and were maintained in a normal 12 hour light/dark cycle. Subretinal injections were performed on 3-4 weeks old mice. Mice were anesthetized with isoflurane inhalation. Proparacaine (0.5%) was applied topically on the cornea and the eyes were dilated with drops of tropicamide (1%) and phenylephrine (2.5%). Eyes were kept lubricated with genteal gel during the surgery. Under a surgical microscope, an ultrafine 30 ½-gauge disposable needle was passed through the sclera, at the equator and next to the limbus, to create a small hole into the vitreous cavity. Using a blunt-end needle, 1-1.5 μL of virus was injected directly into the subretinal space, between the RPE and retinal layer. Each mouse from the experimental groups was injected with 1.5.0e+9 viral genome (vg)/eye.

NGS analysis: 3 weeks post-injection, animals were sacrificed and the eyes enucleated in fresh PBS. Whole retinae were isolated from the eye cups and processed for gDNA extraction using the DNeasy Blood & Tissue Kit (Qiagen) according to the manufacturer's instructions. Amplicons were amplified from 200 ng of gDNA with a set of primers (Fwd 5′-ACACTCTTTCCCTACACGACGCTCTTCCGATCTNNNNNNNGCAGCCTTGGTCTCTGT CTACG-3′ (SEQ ID NO: 40595); Rev 5′-GTGACTGGAGTTCAGACGTGTGCTCTTCCGATCTGCCCCAGTCTCTCTGCTCATACC-3′ (SEQ ID NO: 40596)) targeting the mouse RHO, exon 1 locus, bead-purified (Beckman coulter, Agencourt Ampure XP) and then re-amplified to incorporate illumina adapter sequence. Specifically, these primers contained an additional sequence at the 5′ ends to introduce Illumina read and 2 sequences, as well as a 16 nt random sequence that functions as a unique molecular identifier (UMI). Quality and quantification of the amplicon was assessed using a Fragment Analyzer DNA analyzer kit (Agilent, dsDNA 35-1500 bp). Amplicons were sequenced on the Illumina Miseq according to the manufacturer's instructions. Raw fastq files from sequencing were processed as follows: (1) the sequences were trimmed for quality and for adapter sequences using the program cutadapt (v. 2.1); (2) the sequences from read 1 and read 2 were merged into a single insert sequence using the program flash2 (v2.2.00); and (3) the consensus insert sequences were run through the program CRISPResso2 (v 2.0.29), along with the expected amplicon sequence and the spacer sequence. This program quantifies the percent of reads that were modified in a window around the 3′ end of the spacer (30 bp window centered at −3 bp from 3′ end of spacer). The activity of the CasX molecule was quantified as the total percent of reads that contain insertions, substitutions and/or deletions anywhere within this window.

### Results:

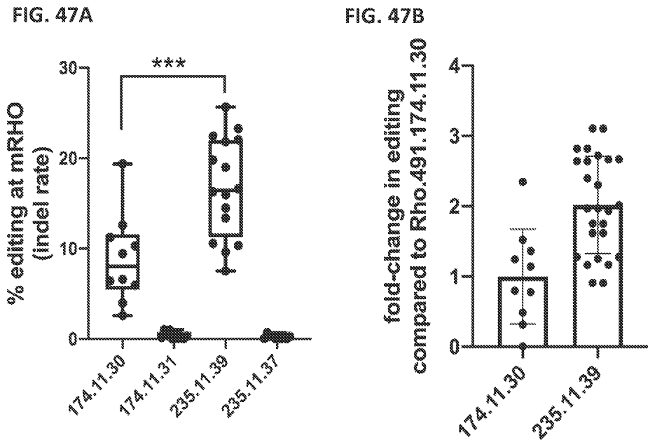

The benchmark vector, AAV.491.174.11.30 (on-target) achieved ˜8% editing across all samples (FIG. 47A; n=8 retinas). A similar vector with spacer 11.31 (off-target, 1 bp mismatch from 11.30 targeting P23H-RHO SNP) showed background level of editing (˜0.4%). An AAV vector expressing scaffold variant 235 and spacer 11.39 achieved over a 2-fold improvement relative to the AAV.491.174.11.30 parental vector (FIG. 47B), with a mean of 16% editing, and as high as 25% in some retinas. This increase in on-target editing remained selective, as no increase in off-target with spacer 11.37 (targeting P23H-RHO SNP, 1 bp-mismatch compared to spacer 11.39) levels compared to AAV.491.174.11.31 parental vector.

These experiments demonstrate proof-of-concept that CasX 491 expression driven by a rod photoreceptor-selective promoter with scaffold 174, and a spacer targeting the mouse P23 RHO locus can achieve therapeutic-relevant levels of edits at the P23 mouse locus when subretinally delivered via AAV in the murine retina. These results also support that editing levels achieved from engineered sgRNA guide (235) and spacer variants (11.39) screened previously in vitro translate as well in vivo, and retain allele-specific selectivity. This study further validates the use of guide scaffold 235 in AAV vectors designed for P23H RHO rescue and genotoxic studies, as well as for other therapeutic targets.

The results of Examples 11 and 12 support that scaffold variants with structural mutation can be engineered with increased activity in dual reporter systems investigating therapeutically relevant genomic targets such as the mouse and human RHO exon 1 loci. Furthermore, while the newly characterized 235 scaffold displayed an overall >2-fold increase in activity, no off-target cleavage with 1-bp mismatch spacer region was detected. This is relevant for allele-specific therapeutic strategy such as adRP P23H Rho, which mutated allele differs from WT sequence by 1 nucleotide, targeted by spacer 11.31. The present study was conducted to further validate the use of guide scaffold 235 in AAV vectors designed for mouse P23H RHO rescue and genotoxic studies, as well as for other therapeutic targets.

### Example 14: Improved CasX Variants Demonstrate Enhanced On-Target Activity In Vitro

The CasX protospacer adjacent motif allows for genomic targeting with precision, which is necessary for various genome editing therapeutic applications, such as autosomal dominant RHO, which requires an allele-specific targeting of the P23H mutation without altering the wild-type sequence.

Experiments were conducted to investigate whether rationally-designed engineered CasX nucleases, with introduced mutations predicted to increase CTC-PAM mediated on-target activity while keeping fidelity high, and with reduced off-target events, improved editing levels at the endogenous mouse RHO locus when delivered in vivo to rod photoreceptors cells,

Additionally, experiments were conducted to further validate the use of guide scaffold 235 in AAV vectors designed for mouse P23H RHO rescue and genotoxic studies, as well as for other therapeutic targets.

### Methods:

CasX protein variants identified in different assays looking at PAM activity were selected for their increased activity at CTC PAM. The CasX proteins were cloned into an AAV transgene construct for plasmid and viral vector validation. We conceptually broke up the AAV transgene between ITRs into different parts, which consisted of our therapeutic cargo and accessory elements relevant to expression in mammalian cells and our nuclease-guide RNA complex (Protein, scaffold, spacer).

Cloning: Each part in the AAV genome was separated by restriction enzyme sites to allow for modular cloning. Parts were ordered as gene fragments from Twist, PCR amplified, and digested with corresponding restriction enzymes, cleaned, then ligated into a vector also digested with the same enzymes. New AAV constructs were then transformed into chemically competent E coli (Stbl3s). Validated constructs were maxi-prepped. To assess the quality of maxi-preps, constructs were processed in two separate digests with XmaI (which cuts at several sites in each of the ITRs) and XhoI which cuts once in the AAV genome. These digests and the uncut construct were then run on a 1% agarose gel. If the plasmid was >90% supercoiled, the correct size, and the ITRs were intact, the construct moved on to be tested via nucleofection and subsequently used for AAV vector production.

Reporter cell lines: An immortalized neural progenitor cell line isolated from the Ai9-tdTomato was cultured in suspension in pre-equilibrated mNPC medium (DMEM/F12 with GlutaMax, 10 mM HEPES, 1×MEM Non-Essential Amino Acids, 1× penicillin/streptomycin, 1:1000 2-mercaptoethanol, 1× B-27 supplement, minus vitamin A, 1× N2 with supplemented growth factors bFGF and EGF. Prior to testing, cells were lifted using accutase, with gentle resuspension, monitoring for complete separation of the neurospheres. Cells were then quenched with media, spun down and resuspended in fresh media. Cells were counted and directly used for nucleofection or 10,000 cells were plated in a 96-well plate coated with PLF (1× Poly-DL-ornithine hydrobromide, 10 mg/mL in sterile diH20, 1× Laminin, and 1× Fibronectin), 2 days prior to AAV transduction.

A HEK293T dual reporter cell line was generated by knocking into HEK293T cells two transgene cassettes that constitutively expressed exon 1 of the human RHO gene linked to GFP and exon 1 of the human P23H.RHO gene linked to mscarlet. The modified cells were expanded by serial passage every 3-5 days and maintained in Fibroblast (FB) medium, consisting of Dulbecco's Modified Eagle Medium (DMEM; Corning Cellgro, #10-013-CV) supplemented with 10% fetal bovine serum (FBS; Seradigm, #1500-500), and 100 Units/mL penicillin and 100 mg/mL streptomycin (100×-Pen-Strep; GIBCO #15140-122), and can additionally include sodium pyruvate (100×, Thermofisher #11360070), non-essential amino acids (100× ThermoFisher #11140050), HEPES buffer (100× ThermoFisher #15630080), and 2-mercaptoethanol (1000× ThermoFisher #21985023). The cells were incubated at 37° C. and 5% CO2. After 1-2 weeks, GFP+/mscarlet+ cells were bulk sorted into FB medium. The reporter lines were expanded by serial passage every 3-5 days and maintained in FB medium in an incubator at 37° C. and 5% C02. Reporter clones were generated by a limiting dilution method. The clonal lines were characterized via flow cytometry, genomic sequencing, and functional modification of the RHO locus using a previously validated RHO targeting CasX molecule. The optimal reporter lines were identified as ones that: i) had a single copies of WT-RHO.GFP and P23H-RHO.mscarlet correctly integrated per cell; ii) maintained doubling times equivalent to unmodified cells; and iii) resulted in reduction in GFP and mscarlet fluorescence upon disruption of the RHO gene when assayed using the methods described below.

Plasmid nucleofection: AAV cis-plasmids driving expression of the CasX-scaffold-guide system were nucleofected in mNPCs using the Lonza P3 Primary Cell 96-well Nucleofector Kit. For the ARPE-19 line, the Lonza SF solution and supplement was used. Plasmids were diluted to concentrations of 200 ng/ul, 100 ng/μL. 5 μL of DNA per construct was added to the P3 or SF solution containing 200,000 tdTomato mNPCs or ARPE-19 cells respectively. The combined solution was nucleofected using a Lonza 4D Nucleofector System according to manufacturer's guidelines. Following nucleofection, the solution was quenched with appropriate culture media. The solution was then aliquoted in triplicate (approx. 67,000 cells per well) in a 96-well plate. 48 hours after transfection, treated cells were replenished with fresh mNPC media containing growth factors. 5 days after transfection, tdTomato mNPCs were lifted and activity was assessed by FACS.

AAV vectors production: Suspension HEK293T cells were adapted from parental HEK293T and grown in FreeStyle 293 media. For screening purposes, small scale cultures (20-30 mL cultured in 125 mL Erlenmeyer flasks and agitated at 110 rpm) were diluted to a density of 1.5e+6 cells/mL on the day of transfection. Endotoxin-free pAAV plasmids with the transgene flanked by ITR repeats were co-transfected with plasmids supplying the adenoviral helper genes for replication and AAV rep/cap genome using PEIMax (Polysciences) in serum-free OPTIMEM media. Cultures were supplemented with 10% CDM4HEK293 (HyClone) 3 hours post-transfection. Three days later, cultures were centrifuged at 1000 rpm for 10 minutes to separate the supernatant from the cell pellet. The supernatant was mixed with 40% PEG 2.5M NaCl (8% final concentration) and incubated on ice for at least 2 hours to precipitate AAV viral particles. The cell pellet, containing the majority of the AAV vectors, was resuspended in lysis media (0.15M NaCl, 50 mM Tris HCl, 0.05% Tween, pH 8.5), sonicated on ice (15 seconds, 30% amplitude) and treated with Benzonase (250 U/μL, Novagen) for 30 minutes at 37° C. Crude lysate and PEG-treated supernatant were then spin at 4000 rpm for 20 minutes at 4° C. to resuspend the PEG precipitated AAV (pellet) with cell debris-free crude lysate (supernatant). clarified further using a 0.45 μM filter.

To determine the viral genome titer, 1 μL from crude lysate viruses was digested with DNase and ProtK, followed by quantitative PCR. 5 μL of digested virus was used in a 25 μL qPCR reaction composed of IDT primetime master mix and a set of primer and 6′FAM/Zen/IBFQ probe (IDT) designed to amplify the CMV promoter region (Fwd 5′-CATCTACGTATTAGTCATCGCTATTACCA-3′ (SEQ ID NO: 40801)); Rev 5′-GAAATCCCCGTGAGTCAAACC-3′ (SEQ ID NO: 40802)), Probe 5′-TCAATGGGCGTGGATAG-3′ (SEQ ID NO: 40803)) or a 62 nucleotide-fragment located in the AAV2-ITR (Fwd 5′-GGAACCCCTAGTGATGGAGTT-3′ (SEQ ID NO: 40804); Rev 5′-CGGCCTCAGTGAGCGA-3′ (SEQ ID NO: 40805), Probe 5′-CACTCCCTCTCTGCGCGCTCG-3′). Ten-fold serial dilutions (5 μl each of 2e+9 to 2e+4 DNA copies/mL) of an AAV ITR plasmid was used as reference standards to calculate the titer (viral genome (vg)/mL) of viral samples. QPCR program was set up as: initial denaturation step at 95′C for 5 minutes, followed by 40 cycles of denaturation at 95′C for 1 min and annealing/extension at 60° C. for 1 min.

AAV transduction: 10,000 cells/well of mNPCs were seeded on PLF-coated wells in 96-well plates 48-hours before AAV transduction. All viral infection conditions were performed in triplicate, with normalized number of vg among experimental vectors, in a series of 3-fold dilution of multiplicity of infection (MOI) ranging from ˜1.0e+6 to 1.0e+4 vg/cell. Calculations were based on an estimated number of 20,000 cells per well at the time of transfection. Final volumes of 50 μL of AAV vectors diluted in pre-equilibrated mNPC medium supplemented with bFGF/EGF growth factors (20 ng/ml final concentration) were applied to each well. 48 hours post-transfection, complete media change was performed with fresh media supplemented with growth factors. Editing activity (tdT+ cell quantification) was assessed by FACS 5 days post-transfection.

Assessing editing activity by FACS: 5 days after transfection, treated tdTomato mNPCs or ARPE-19 cells in 96-well plates were washed with dPBS and treated with 50 μL TrypLE and Trypsin (0.25%) for 15 and 5 minutes, respectively. Following cell dissociation, treated wells were quenched with media containing DMEM, 10% FBS and 1× penicillin/streptomycin. Resuspended cells were transferred to round-bottom 96-well plates and centrifuged for 5 min at 1000× g. Cell pellets were then resuspended with dPBS containing 1×DAPI, and plates were loaded into an Attune NxT Flow Cytometer Autosampler. The Attune NxT flow cytometer was run using the following gating parameters: FSC-A×SSC-A to select cells, FSC-H×FSC-A to select single cells, FSC-A×VL1-A to select DAPI-negative alive cells, and FSC-A×YL1-A to select tdTomato positive cells.

NGS analysis of indels at mRHO exon 1 locus: 5 days after transfection, treated tdTomato mNPCs in 96-well plates were washed with dPBS and treated with 50 μL TrypLE and trypsin (0.25%) for 15 and 5 minutes, respectively. Following cell dissociation, treated wells were quenched with media containing DMEM, 10% FBS and 1× penicillin/streptomycin. Cells were then spun down and resulting cell pellets washed with PBS prior to processing them for gDNA extraction using the Zymo mini DNA kit according to the manufacturer's instructions. For assessing editing levels occurring at the mouse RHO exon 1 locus, amplicons were amplified from 200 ng of gDNA with a set of primers (Fwd 5′-ACACTCTTTCCCTACACGACGCTCTTCCGATCTNNNNNNNGCAGCCTTGGTCTCTGT CTACG-3′ (SEQ ID NO: 40595); Rev 5′-GTGACTGGAGTTCAGACGTGTGCTCTTCCGATCTGCCCCAGTCTCTCTGCTCATACC-3′ (SEQ ID NO: 40596)), bead-purified (Beckman coulter, Agencourt Ampure XP) and then re-amplified to incorporate illumina adapter sequence. Specifically, these primers contained an additional sequence at the 5′ ends to introduce Illumina read and 2 sequences as well as a 16 nt random sequence that functions as a unique molecular identifier (UMI). Quality and quantification of the amplicon was assessed using a Fragment Analyzer DNA analyzer kit (Agilent, dsDNA 35-1500 bp). Amplicons were sequenced on the Illumina Miseq according to the manufacturer's instructions. Raw fastq files from sequencing were processed as follows: (1) the sequences were trimmed for quality and for adapter sequences using the program cutadapt (v. 2.1); (2) the sequences from read 1 and read 2 were merged into a single insert sequence using the program flash2 (v2.2.00); and (3) the consensus insert sequences were run through the program CRISPResso2 (v 2.0.29), along with the expected amplicon sequence and the spacer sequence. This program quantifies the percent of reads that were modified in a window around the 3′ end of the spacer (30 bp window centered at −3 bp from 3′ end of spacer). The activity of the CasX molecule was quantified as the total percent of reads that contain insertions, substitutions and/or deletions anywhere within this window

### Results:

Engineered mutations in prior assays identified CasX variants with the ability to increase both overall activity, specificity of the nuclease, as well as increased activity with spacers targeting CTC-PAM sites. These mutations to the CasX 491 protein gave rise to CasX variant proteins 515, 527, 528, 535, 536 and 537 (see Table 3 for sequences).

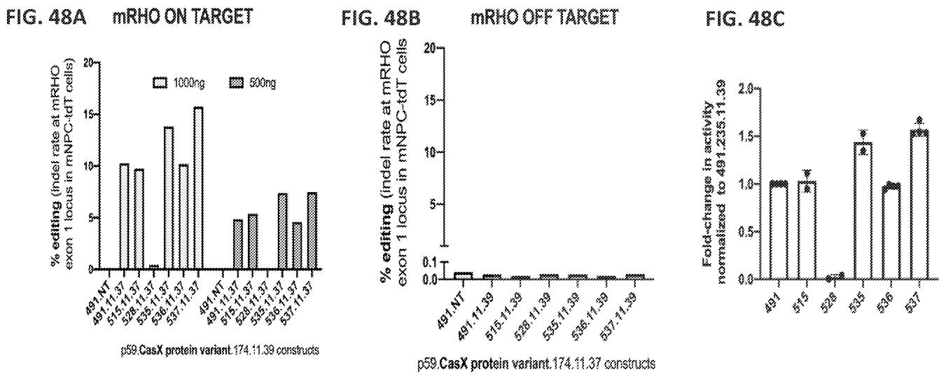

Multiple editing screens were conducted to quantify on-target editing levels mediated by these CasX variant proteins paired with gRNA scaffolds 174 or 235 and different spacers targeting multiple genomic loci of interest (the encoding sequences of the guides and spacers are presented in Tables 18 and 19). Constructs were cloned into the AAV backbone p59, flanked by ITR2 sequences, driving expression of the Cas X under the control of a CMV promoter, as well the scaffold-spacer under the control of the human U6 promoter. The mNPC-tdT reporter cell line was used to assess single-cut efficiency at the endogenous mouse RHO exon 1 locus (spacer 11.39, CTC PAM, FIG. 48A). A dual reporter system integrated in a ARPE-19 derived cell line was also used to assess on-target editing at the exogenously expressed human WT Rho locus (spacer 11.41, CTC PAM) or at the P23H-RHO locus (spacer 11.43, CTC PAM, FIG. 48B).

The CasX protein variants with spacer 11.39 were tested via nucleofection in the mouse NPC cell line at two different doses, 1000 ng and 500 ng. Constructs were compared to the parental CasX 491 activity. AAV constructs expressing CasX 535 and 537 with scaffold 174 and spacer 11.30 demonstrated the greatest editing activity at the mRHO exon 1 locus of any of the CasX variants (by percent editing, FIG. 48A), which was increased 1.5-fold relative to CasX 491 (FIG. 48C, normalized to 1), without increased off-target cleavage, shown by the nucleofection of the protein variants with spacer 11.37 (targeting mutant P23H-Rho allele, FIG. 48B).

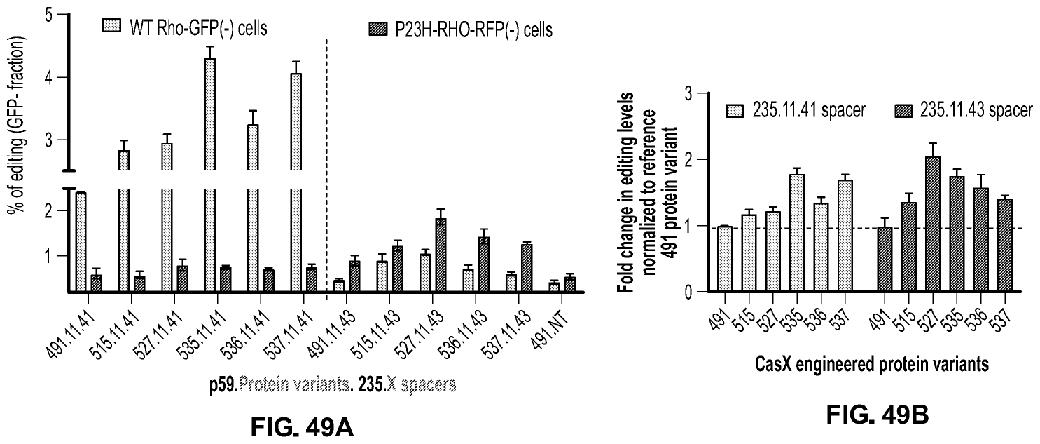

Experiments were then conducted to determine whether the improvements observed at the mouse RHO locus with the mutated variants translated at the human RHO locus, which is more clinically-relevant. The dual reporter ARPE-19 cell line was nucleofected with constructs expressing the CasX variant proteins paired with either sgRNA-scaffold 235 with spacer 11.41 or spacer11.43, targeting human RHO. CasX 535 and 537 also displayed over 1.5-fold increased editing activity compared to CasX 491 (˜4.3% and 4.1% editing compared to 2.4% editing of Rho-GFP− cells respectively, FIGS. 49A and 49B) when targeting the exogenous WT-RHO-GFP locus. Constructs expressing CasX variants 515, 527 and 536 edited at similar levels to CasX 491. Interestingly, when using a spacer targeting the P23H-RHO-mscarlet locus, all the variant proteins demonstrated improved editing compared to CasX 491. The highest activity levels were achieved by constructs expressing CasX 527 (2-fold increase) and CasX 535 (1.8-fold increase).

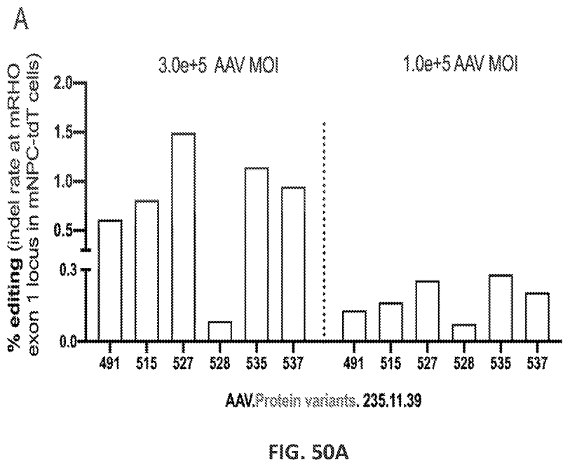

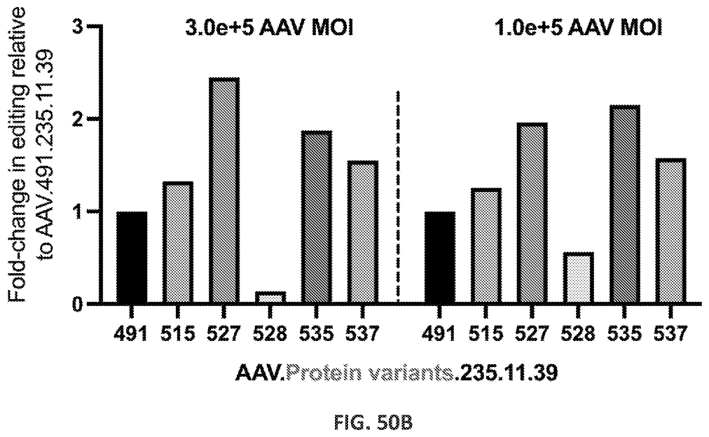

Finally, we sought to demonstrate that these protein variants packaged efficiently in AAV and remained potent when delivered virally. mNPC transduced with AAV vectors expressing CasX 527, 535 and 537 and guide scaffold 235 with spacer 11.39 (on target, mouse WT RHO) showed increased activity at the on-target locus (>2-fold increase, FIGS. 50A and 50B) relative to AAV CasX 491 and guide scaffold 235 with spacer 11.39 with transduction at 3.0e+5 MOI. Fold-improvement in activity were observed in a dose-dependent manner.

These results support that CasX variants with structural mutations can be engineered resulting in increased editing activity in dual reporter systems at therapeutically-relevant genomic targets, such as the mouse and human RHO exon 1 loci. Furthermore, while the newly-characterized variants displayed an overall 1.5-2-fold increase in activity, they retained allele-specific targeting with no off-target cleavage detected with a 1-bp mismatch spacer. This is relevant for allele-specific therapeutic strategy, such as editing at adRP P23H Rho, where the mutated allele differs from WT sequence by 1 nucleotide (targeted by spacer 11.37). This study further validates the use of CasX variants 527, 535, 536 with scaffold 235 in AAV vectors designed for P23H RHO rescue and genotoxic studies, as well as for other therapeutic targets.

### Example 15: AAV Constructs with CasX and Targeted Guides Edit the P23 RHO Locus In Vivo in C57BL/6J Mice

Experiments were conducted to demonstrate the ability of CasX to edit in vivo the endogenous RHO locus in the mouse retina, with a spacer targeting the P23 residue at a therapeutically relevant level, to generate proof-of-concept data that will justify and inform experiments in the P23H mouse disease model. Here, we assessed whether CasX variant 491 and guide variant 174, and a spacer targeting the P23 locus of the mouse RHO gene can generate significant, detectable in the retina when injected subretinally, and evaluate efficacy and safety of two different viral doses (1.0e+9 and 1.0e+10 vg). Rescue of 10% of rod photoreceptors can restore vision in cases of AdRP. Therefore, editing 10% of the RHO loci in rod photoreceptors in the retina may provide a therapeutic benefit in a disease context by reducing the levels of the mutant rhodopsin protein and preventing rod photoreceptor degeneration.

### Materials and Methods:

### Generation of AAV Plasmids and Viral Vectors

The CasX variant 491 under the control of the CMV promoter and RNA guide variant 174/spacer 11.30 (AAGGGGCTCCGCACCACGCC (SEQ ID NO: 40502), targeting mouse RHO exon 1 at P23 residues) under the U6 promoter were cloned into a pAAV plasmid flanked with AAV2 ITR. AAV.491.174.11.30 vectors were produced in HEK293 cells using the triple-transfection method.

### Subretinal Injections

C57BL/6J mice were obtained from the Jackson Laboratories and maintained in a normal 12 hour light/dark cycle. Subretinal injections were performed on 5-6 weeks old mice. Mice were anesthetized with isoflurane inhalation. Proparacaine (0.5%) was applied topically on the cornea and the eyes were dilated with drops of tropicamide (1%) and phenylephrine (2.5%). Eyes were kept lubricated with genteal gel during the surgery. Under a surgical microscope, an ultrafine 30 ½-gauge disposable needle was passed through the sclera, at the equator and next to the limbus, to create a small hole into the vitreous cavity. Using a blunt-end needle, 1-1.5 μL of virus was injected directly into the subretinal space, between the RPE and retinal layer. Each experimental group (n=5) were injected in one eye with 1e+9 vg or 1e+10 viral genome (vg)/eye, and the contralateral eye injected with the AAV formulation buffer.

### NGS Analysis

3 weeks post-injection, animals were sacrificed and the eyes enucleated in fresh PBS. Whole retinae were isolated from the eye cups and processed for gDNA extraction using the DNeasy Blood & Tissue Kit (Qiagen) according to the manufacturer's instructions. Amplicons were amplified from 200 ng of gDNA with a set of primers (Fwd 5′-ACACTCTTTCCCTACACGACGCTCTTCCGATCTNNNNNNNGCAGCCTTGGTCTCTGT CTACG-3′ (SEQ ID NO: 40595); Rev 5′-GTGACTGGAGTTCAGACGTGTGCTCTTCCGATCTGCCCCAGTCTCTCTGCTCATACC-3′ (SEQ ID NO: 40596)) targeting the mouse RHO, exon 1 locus, bead-purified (Beckman coulter, Agencourt Ampure XP) and then re-amplified to incorporate illumina adapter sequence. Specifically, these primers contained an additional sequence at the 5′ ends to introduce Illumina read and 2 sequences as well as a 16 nt random sequence that functions as a unique molecular identifier (UMI). Quality and quantification of the amplicon was assessed using a Fragment Analyzer DNA analyzer kit (Agilent, dsDNA 35-1500 bp). Amplicons were sequenced on the Illumina Miseq according to the manufacturer's instructions. Raw fastq files from sequencing were processed as follows: (1) the sequences were trimmed for quality and for adapter sequences using the program cutadapt (v. 2.1); (2) the sequences from read 1 and read 2 were merged into a single insert sequence using the program flash2 (v2.2.00); and (3) the consensus insert sequences were run through the program CRISPResso2 (v 2.0.29), along with the expected amplicon sequence and the spacer sequence. This program quantifies the percent of reads that were modified in a window around the 3′ end of the spacer (30 bp window centered at −3 bp from 3′ end of spacer). The activity of the CasX molecule was quantified as the total percent of reads that contain insertions, substitutions and/or deletions anywhere within this window.

### Immunohistology:

Mice were euthanized 3-4 weeks post-injection. Enucleated eyes were placed in 10% formalin overnight at 4° C. Retinae were dissected out from the eye cups, rinsed in PBS thoroughly and immersed in 15%-30/o sucrose gradient. Tissues were embedded in optimal cutting temperature (OCT), froze on dry ice before being transferred to ˜80′C storage. 20 μM sections were cut using a cryostat. The sections were blocked for ≥1 hour at room temperature in blocking buffer (2% normal goat serum, 1% BSA, 0.1% Triton-X 100) before antibody labeling. The antibodies used were anti-mouse HA (abcam, 1:500) and Alexa Fluor 488 rabbit anti-mouse (Invitrogen, 1:2000). Sections were counterstained with DAPI to label nuclei, mounted on slides and imaged on a fluorescent microscope.

### Results:

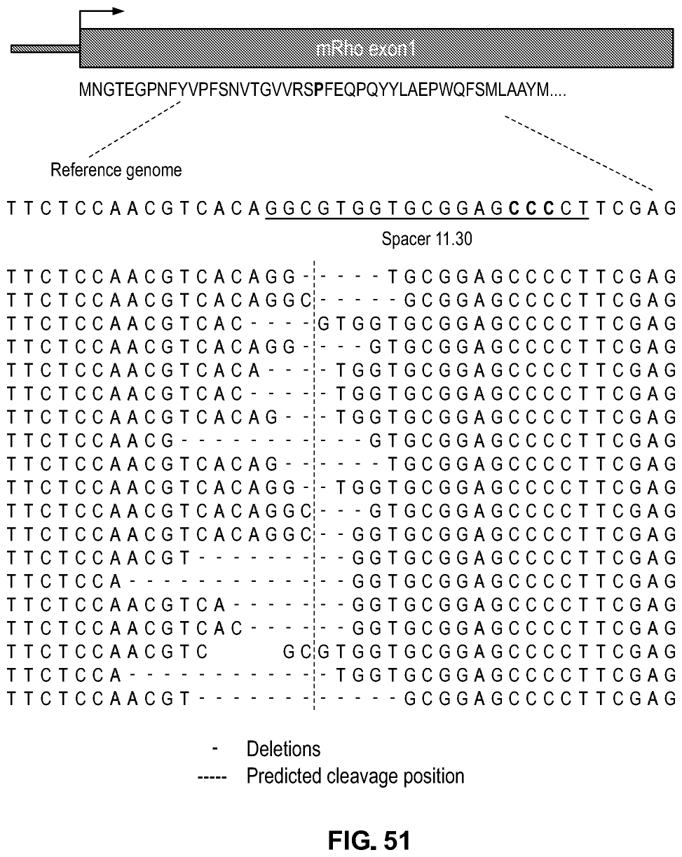

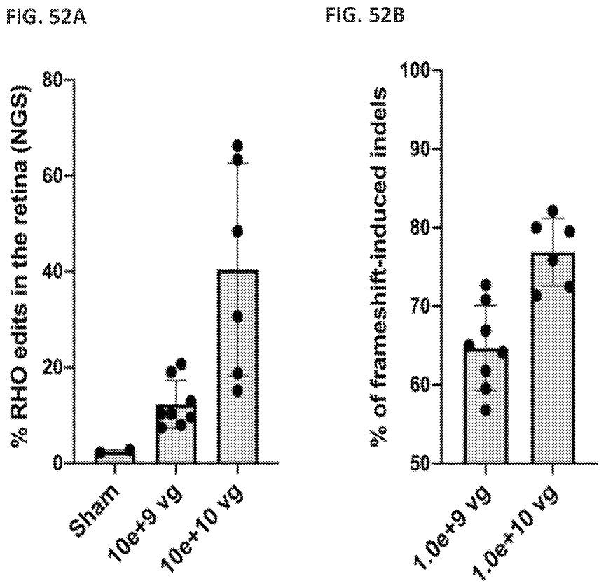

We assessed the ability of CasX to edit the P23 RHO locus in the mouse retina. Two therapeutically relevant doses, 1.0e+9 and 1.0E+10 vg of AAV-CasX.491.174.11.30 were administered in the subretinal space of 5-6 weeks old C57BL/6J mice. Three weeks post-injections, retinae were harvested and editing levels quantified via NGS and the CRISPResso analysis pipeline. The spacer 11.30 targets the WT P23 genomic locus (FIG. 51) located at the beginning of the first exon of RHO. Overexpression of CasX-491.174.11.30 led to significant, dose-dependent, editing of mRHO exon 1 locus in treated-compared to sham-injected retinae (FIGS. 52A-52B). The left panel (FIG. 52A) shows the quantification in % of total indels detected by NGS at the mouse P23 RHO locus in AAV-CasX or sham-injected retinae compared to the mouse reference genome. The right panel (FIG. 52B) shows the fraction (%) of edits predicted to lead to frameshift mutations in RHO protein. Data are presented as average of NGS readouts of editing outcomes from the entire retina, from six to eight animals per experimental cohort. The highest AAV dose, 1e+10 vg/eye, increased indels rate by 4-fold compared to the 1.0e+9 vg dose, with 40.3±22% versus 12.3±5% RHO editing detected respectively. The majority of indels generated by CasX.491 were deletions (left panel), predicted to translate to a high frequency of frameshift-mutations (64.7 versus 76.9% for 1.0e+9 and 1.0e+10 vg/dose respectively), and hypothetically high levels of RHO protein knock down. These results suggest that with a spacer driving allele-specific target of mutant P23H locus in the P23H+/− mouse model, CasX could efficiently editing 10% of rod photoreceptor, with the majority of edits translating to a knocking-down the mutant P23H Rho and significantly delay photoreceptor degeneration.

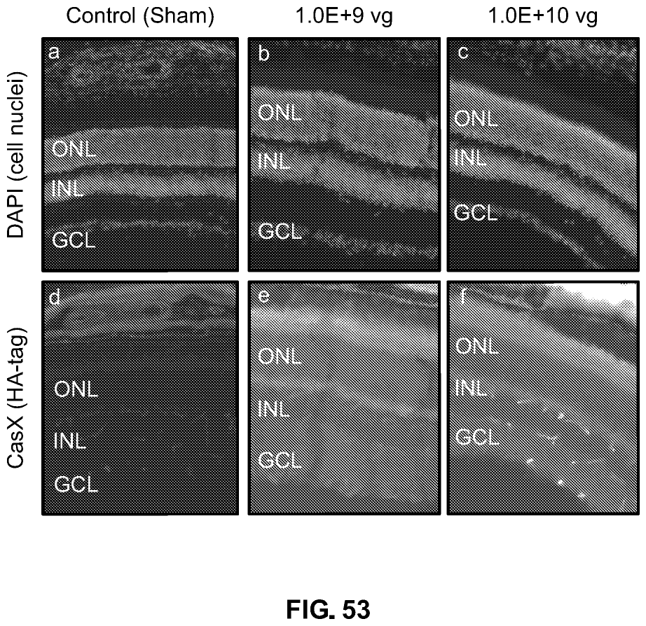

Immunohistochemistry performed on injected retinal cross-sectioned confirmed CasX expression in the photoreceptors layers, but also showed spread of the virus to the inner layers as show in in FIGS. 53A-53F. The treatment groups were 1.0e+9 vg of AAV-CasX (FIGS. 53B and 53E); 1.0e+10 vg AAV-CasX (FIGS. 53C and 53F); or PBS (FIGS. 53A and 53D). Levels of HA-tagged CasX was assessed by Anti-HA antibody staining (lower panels of FIGS. 53E, and 53F) in the photoreceptor cell bodies in the located in the outer nuclear layer (ONL) as well as outer segments, in retinas injected with both the 1e9 vg (FIGS. 53B and 53E) and 1e10 vg (FIGS. 53C and 53F). The control retinas that received a sham (FIGS. 53A and 53C) injection only showed background levels of signal for HA staining (FIG. 53D) in the RPE/sclera and had no detectable level in the ONL/INL layer. Additionally, gross histological analysis showed that the retinal structure was maintained after subretinal administration of AAV packaging CasX constructs.

Under the conditions of the experiments, the results demonstrate proof-of-concept that CasX 491, scaffold 174, and a spacer targeting the mouse P23 RHO locus can achieve therapeutically-relevant levels of edits at the P23 mouse locus when subretinally delivered via AAV in the murine retina.

### Example 16: AAV-Mediated Selective Expression of CasX in Photoreceptors Result in Strong On-Target Activity In Vivo by NGS and Structural Analysis

Experiments were conducted to demonstrate the ability of CasX to edit selectively photoreceptors in the mouse retina by restricting its expression with a selective photoreceptor promoter, with a spacer targeting the P23 residue at a therapeutically relevant level in the wild-type retina. We further show strong correlation between editing and proteomic levels in a transgenic reporter model expressing GFP only in rod photoreceptors. Here, we assessed whether CasX variant 491 and guide variant 174 with a spacer targeting the integrated GFP locus generated significant, detectable editing levels in the retina when injected subretinally, and evaluated the efficacy of two different viral doses (1.0e+9 and 1.0e+10 vg per eye).

### Methods:

Generation of AAV Plasmids and Viral Vectors: The CasX variant 491 under the control of the various photoreceptor-specific promoters (RP1, RP2, RP3 based on endogenous rhodopsin RHO promoter, and RP4, RP5 based on endogenous G-coupled Retinal Kinase GRK1 promoter; sequences in Table 22) as well as the CMV promoter, and the sgRNA guide variant 174/spacer 11.30 (AAGGGGCTCCGCACCACGCC (SEQ ID NO: 40502)), targeting mouse RHO exon 1 at P23 residues) under the U6 promoter were cloned into pAAV plasmid flanked with AAV2 ITR. A WPRE sequence amplified with EcoRI restriction sites on each side was inserted into EcoRI digested p59.RP4.491.174.11.30, and p59.RP5.491.174.11.30 plasmids. For the efficacy study in the Nrl-GFP model, spacer 4.76 (TGTGGTCGGGGTAGCGGCTG (SEQ ID NO: 17)) targeting GFP was cloned into AAV-cis plasmid p59.RP1.491.174 using the Golden Gate cloning with bbsI restriction sites flanking the spacer region.

TABLE 22
Rho promoter sequences

| Promoter | PR construct | SEQ ID NO: | DNA Sequence |
|---|---|---|---|
| RHO | RP1 | 40589 | ND |
| RHO535-CAG | RP2 | 40590 | ND |
| RHO-intron | RP3 | 40591 | ND |
| GRK | RP4 | 40592 | ND |
| GRK-SV40 | RP5 | 40593 | ND |
| GRK-CAG | RP6 | 40594 | ND |

* ND = no description, sequence provided in sequence listing.

AAV vector production: Suspension HEK293T cells were adapted from parental HEK293T and grown in FreeStyle 293 media. 500 mL cultures (1 L Erlenmeyer flasks, agitated at 110 rpm) were diluted to a density of 2e+6 cells/mL on the day of transfection. Endotoxin-free pAAV plasmids with the transgene flanked by ITR repeats were co-transfected with plasmids supplying the adenoviral helper genes for replication and AAV rep/cap genome using PEIMax (Polysciences) in serum-free OPTIMEM media. Cultures were supplemented with 10% CDM4HEK293 (HyClone) 3 hours post-transfection. Three days later, cultures were centrifuged at 1000 rpm for 10 minutes to separate the supernatant from the cell pellet. The supernatant was mixed with 40% PEG 2.5M NaCl (8% final concentration) and incubated on ice for at least 2 hours to precipitate AAV viral particles. The cell pellet, containing the majority of the AAV vectors, was resuspended in lysis media (0.15M NaCl, 50 mM Tris HCl, 0.05% Tween, pH 8.5), sonicated on ice (15 seconds, 30% amplitude) and treated with Benzonase (250 U/μL, Novagen) for 30 minutes at 37° C. Crude lysate and PEG-treated supernatant were then spin at 4000 rpm for 20 minutes at 4° C. to resuspend the PEG precipitated AAV (pellet) with cell debris-free crude lysate (supernatant) clarified further using a 0.45 μM filter. AAV lysates were purified using affinity chromatography (POROS CaptureSelect AAVX, ThermoFisher). Eluate was buffer exchanged and concentrated in PBS+200 mM NaCl+0.001% Pluronic. To determine the viral genome titer, 1 μL from crude lysate viruses was digested with DNase and ProtK, followed by quantitative PCR. 5 μL of digested virus was used in a 25 μL qPCR reaction composed of IDT primetime master mix and a set of primer and 6′FAM/Zen/IBFQ probe (IDT) designed to amplify a 62 bp-fragment located in the AAV2-ITR (Fwd 5′-GGAACCCCTAGTGATGGAGTT-3′ (SEQ ID NO: 40804); Rev 5′-CGGCCTCAGTGAGCGA-3′ (SEQ ID NO: 40805), Probe 5′-CACTCCCTCTCTGCGCGCTCG-3′ (SEQ ID NO: 40806)). An AAV ITR plasmid was used as reference standards to calculate the titer (viral genome (vg)/mL) of viral samples. QPCR program was set up as: initial denaturation step at 95′C for 5 minutes, followed by 40 cycles of denaturation at 95′C for 1 min and annealing/extension at 60° C. for 1 min.

The AAV vector AAV.RP1.491.174.4.76 was produced at the University of North Carolina (UNC) Vector Core using the triple transfection methods in HEK239T.

Subretinal injections: C57BL/6J mice and heterozygous Nrl-GFP/C57BL/5J mice (Jackson Laboratories) were maintained in a normal 12 hour light/dark cycle. Subretinal injections were performed on 4-5 week-old mice. Mice were anesthetized with isoflurane inhalation. Proparacaine (0.5%) was applied topically on the cornea and the eyes were dilated with drops of tropicamide (1%) and phenylephrine (2.5%). Eyes were kept lubricated with genteal gel during the surgery. Under a surgical microscope, an ultrafine 30 ½-gauge disposable needle was passed through the sclera, at the equator and next to the limbus, to create a small hole into the vitreous cavity. Using a blunt-end needle, 1-1.5 μL of virus was injected directly into the subretinal space, between the RPE and retinal layer. Each mouse from the experimental groups was injected in one eye with 1.0e+9, 5.0e+9 or 1.0e+10 genome (vg)/eye, and the contralateral eye injected with the AAV formulation buffer.

Western blot: To generate protein lysates, eyes were freshly enucleated and dissected in ice-cold PBS, snap-frozen in dry ice, and resuspended in RIPA buffer (150 mM NaCl, 1% NP40, 0.5% deoxycholate, 0.1% SDS, 50 mM Tris pH8.0, dH20) freshly supplemented with protease inhibitors (5 mg/mL final concentration), DTT and PMSF (final concentration 1 mM respectively) in individual 1.5 mL Eppendorf tube per retina. Retinal tissue was further homogenized in small pieces using a RNA-free disposable pellet pestles (Fisher scientific, #12-141-364) and incubated on ice for 30 minutes, flipping the tube occasionally to gently mix. Samples were then centrifuged at 4° C. at full speed for 20 minutes to pellet genomic DNA. Protein extracts and gDNA cell pellets were then separated. For protein extracts, supernatants were collected. Protein concentration were determined by BCA assay and read on Tecan plate reader. 15 μg of total protein lysate of mouse retina were separated by SDS-PAGE (Bio-Rad TGX gels) and transferred to polyvinylidene difluoride membranes using the Transblot Turbo. The membranes were blocked with 5% nonfat dry milk for 1 h at room temperature and incubated overnight at 4° C. with the primary antibody. Then, blots were washed with Tris-buffered saline with the Tween-20 (137 mM sodium chloride, 20 mM Tris, 0.1% Tween-20, pH 7.6) for three times and incubated with the horseradish peroxidase-conjugated anti-rabbit or anti-mouse secondary antibody for 1 h at room temperature. After washing three times, the membranes were developed using Chemiluminescent substrate ECL and imaged on the ChemicDoc (X). Blot images were processed with ImageLab.

NGS analysis: Animals were sacrificed and the eyes enucleated in fresh PBS. Whole retinae were isolated from the eye cups and processed for gDNA extraction as described previously in western blot section. Genomic gDNA pellets were processed with the DNeasy Blood & Tissue Kit (Qiagen) according to the manufacturer's instructions. Amplicons were amplified from 200 ng of gDNA with a set of primers (Table 23) targeting the genomic region of interest. Amplicons were bead-purified (Beckman coulter, Agencourt Ampure XP) and then re-amplified to incorporate illumina adapter sequence. Specifically, these primers contained an additional sequence at the 5′ ends to introduce Illumina read and 2 sequences as well as a 16 nt random sequence that functions as a unique molecular identifier (UMI). Quality and quantification of the amplicon was assessed using a Fragment Analyzer DNA analyzer kit (Agilent, dsDNA 35-1500 bp). Amplicons were sequenced on the Illumina Miseq according to the manufacturer's instructions. Raw fastq files from sequencing were processed as follows: (1) the sequences were trimmed for quality and for adapter sequences using the program cutadapt (v. 2.1); (2) the sequences from read 1 and read 2 were merged into a single insert sequence using the program flash2 (v2.2.00); and (3) the consensus insert sequences were run through the program CRISPResso2 (v 2.0.29), along with the expected amplicon sequence and the spacer sequence. This program quantifies the percent of reads that were modified in a window around the 3′ end of the spacer (30 bp window centered at −3 bp from 3′ end of spacer). The activity of the CasX molecule was quantified as the total percent of reads that contain insertions, substitutions and/or deletions anywhere within this window.

TABLE 23
NGS primer sequences

| | | SEQ ID | |
|---|---|---|---|
| Primer | Target | NO: | Sequence (5′-3′) |
| 1F | RHO | 40595 | ACACTCTTTCCCTACACGACGCTCTTCCGATCTNNNNN |
| | exon 1 | | NNGCAGCCTTGGTCTCTGTCTACG |
| 1R | RHO | 40596 | GTGACTGGAGTTCAGACGTGTGCTCTTCCGATCTGCCC |
| | exon 1 | | CAGTCTCTCTGCTCATACC |
| 2F | GFP | 40597 | ACACTCTTTCCCTACACGACGCTCTTCCGATCTNNNNN |
| | | | NNNNNNNNNNNGACGTAAACGGCCACAAGTTCAGC |
| 2R | GFP | 40598 | GTGACTGGAGTTCAGACGTGTGCTCTTCCGATCTCGTTC |
| | | | TTCTGCTTGTCGGCCATGA |

Immunohistology: Enucleated eyes were placed in 10% formalin overnight at 4° C. Retinae were dissected out from the eye cups, rinsed in PBS thoroughly and immersed in 15%-30% sucrose gradient. Tissues were embedded in optimal cutting temperature (OCT), frozen on dry ice before being transferred to ˜80′C storage. 20 μM sections were cut using a cryostat. The sections were blocked for >1 hour at room temperature in the blocking buffer (2% normal goat serum, 1% BSA, 0.1% Triton-X 100) before antibody labeling. The antibodies used were: anti-mouse HA (abcam, 1:500); Alexa Fluor 488 rabbit anti-mouse (Invitrogen, 1:2000). Slides were counterstained with Hoechst 33342 (Thermo Fisher Scientific, Hemel Hempstead, UK) and mounted with Prolong Diamond antifade mounting medium (Thermo Fisher Scientific, Hemel Hempstead, UK). Confocal fluorescence imaging was subsequently performed using the LSM-710 inverted confocal microscope system (Carl Zeiss, Cambridge, UK).

### Results:

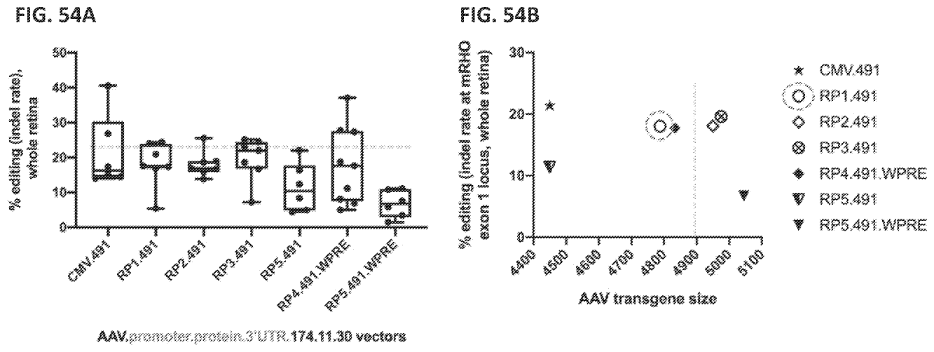

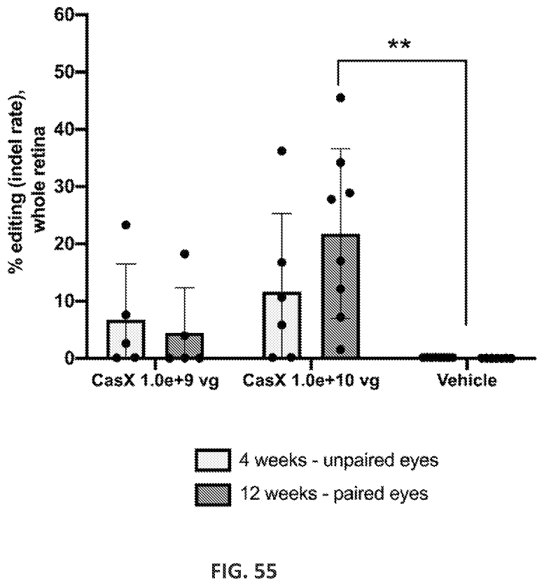

Editing levels were quantified at the mRHO exon locus in 3 week-old C57BL/6J that were injected subretinally with AAV vectors expressing CasX 491 under the control of multiple engineered retinal and ubiquitous promoters to identify promoters driving strong levels of editing in the photoreceptors, with spacer 11.30. Rod-specific RP1, RP2, RP3, RP4 promoters mediated very similar levels of editing (˜20%). Vectors AAV.RP5.491.174.11.30 and AAV RP5.491.WPRE.174.11.30 led to lower expression levels (˜10 and 8% respectively, FIG. 54A). We identified optimized vectors AAV.RP1.491.174.11.30 as most potent vectors for further functional and distribution study, with the goal of achieving high levels of editing in vivo in photoreceptors as well as making the transgene plasmid significantly smaller in size to package within the AAV (100-400 bp shorter than other constructs with similar level of activity (FIG. 54B). This optimized construct was further validated by conducting an efficacy study in a transgenic model expressing GFP in rod photoreceptors, a convenient model used in the field to validate rod-specific or knock down of protein. AAV.RP1.491.174.4.76 vectors were injected at 2 different doses to study efficacy. 4 and 12-weeks post-injections, we quantified levels of editing at the integrated GFP locus by NGS, and observed detectable editing levels. With the 1.0E+9 vg/eye dose arm, we observed ˜8% of editing levels. With the increased dose group injected with 1.0e+10 vg, 10% editing levels were detectable at 4-weeks, which increased by 2-fold in the follow-up time point, 12-weeks post-injections (FIG. 55).

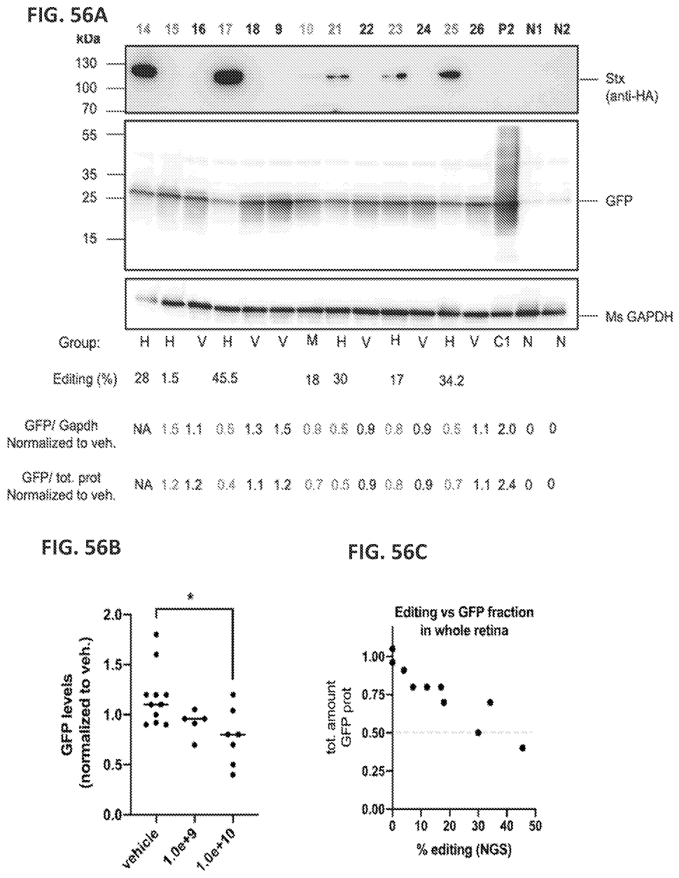

Editing levels were confirmed by structural and proteomic analysis. Western blot analysis of 12-week post-injection retinal lysates showed strong correlation between levels of editing and reduction in GFP protein (FIGS. 56A and 56C), with protein knock-down detected with as low as 5% editing in whole-retina. GFP protein levels were significantly lower than the vehicle group in the AAV-CasX-treated retinas at the 1.0e+10 vg/eye dose (FIG. 56B).

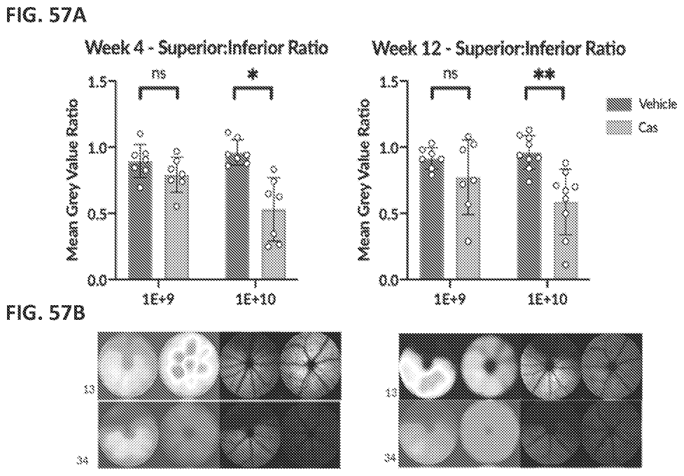

These results were also confirmed by in vivo fundus imaging of GFP fluorescence. The ratio of superior to inferior retina mean grey values showed a reduction in 20% and 50% GFP fluorescence by week 12 (FIG. 57A). A complete decrease in GFP fluorescence over time was visible within the quadrant who received the subretinal injection only in the injected retinas compared to the vehicle group (FIG. 57B).

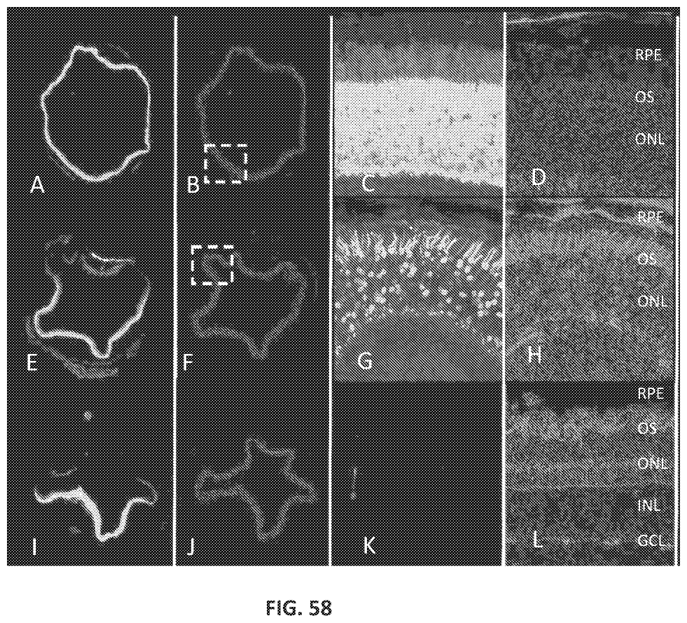

Immunochemistry staining confirmed (FIGS. 58A-58L) the decrease of GFP protein expression in rod photoreceptors. Representative confocal images show strong GFP expression in the retinae injected with only the AAV formulation buffer. Whole retina is expressing GFP, matching with the nuclei staining (FIGS. 58A-C). No HA expression was detectable, as a read-out of AAV-mediated CasX transgene expression (FIG. 58D).

Retinae injected with 1.0e+9 and 1.0e+10 showed strong decrease in GFP expression in whole retina sections, in a dose-dependent manner (FIGS. 58E-58L), which correlated with detectable levels of HA only rod outer segments (OS) and outer nuclear layers (ONL), confirming the promoter RP1 selectivity for rod photoreceptors. High dose treatment resulted in complete knockdown of injected retina (˜50% of GFP knockdown in whole-retina, as injection is limited to the superior gradient) while the 1.0e+9 vg dose decreased ˜50% of GFP expression in localized area (FIGS. 58G and 58K) compared to control (FIG. 58C).

The results demonstrate proof-of-concept that CasX 491, scaffold 174, and a spacer targeting the mouse P23 RHO locus can achieve therapeutic-relevant levels of edits at the P23 mouse locus when only expressed in rod-photoreceptors, the therapeutic cell target, via AAV-mediated subretinal delivery. Further, the specificity and efficacy of the vector was demonstrated by conducting a follow-up study targeting a GFP locus integrated in a reporter model overexpressing GFP in photoreceptors in which the results show a strong correlation between editing levels and protein knock-down assessed by western blot, fundus imaging and histology.

### Example 17: Demonstration that the CasX:gNA System can Edit Human Neural Progenitor Cells and Induced Neurons Efficiently when Packaged and Delivered Via AAVs

Experiments were performed to demonstrate the efficiency of AAV-expressed CasX:gNA system in editing human neural progenitor cells (hNPCs) and induced neurons (iNs) in vitro.

### Materials and Methods:

### AAV Construct Cloning:

CasX variant 491 and guide scaffold variant 235 were used in these experiments.

To evaluate the editing capability of AAV-expressed CasX:gNA system in hNPCs, AAV constructs containing a UbC promoter driving CasX expression and a Pol III promoter scaffold driving the expression of a gRNA with scaffold variant 235 and spacer 7.37 (GGCCGAGAUGUCUCGCUCCG; SEQ ID NO: 379; incorporated in construct ID 183), which targeted the endogenous B2M locus, were generated using standard molecular cloning techniques. Cloned and sequence-validated constructs were maxi-prepped and subjected to quality assessment prior to transfection for AAV production.

For experiments assessing the editing capability of AAV-expressed CasX:gNA system in human iNs, AAV constructs encoding for CasX protein and gRNA with AAVS1-targeting spacer 31.12 (UUCUCGGCGCUGCACCACGU; SEQ ID NO: 41830; incorporated in construct ID 188), 31.63 (CAAGAGGAGAAGCAGUUUGG; SEQ ID NO: 41831; incorporated in construct ID 189), or 31.82 (GGGGCCUGUGCCAUCUCUCG; SEQ ID NO: 41832; construct ID 190), were similarly generated as described. The non-targeting spacer 0.1 (AGGGGUCUUCGAGAAGACCC; SEQ ID NO: 41833) was also used in these experiments. For experiments assessing various protein promoters driving the expression of CasX 491 with gRNA spacer 7.37 to edit the B2M locus in human iNs, AAV constructs containing these protein promoter variants were similarly generated as described (see Table 24 for sequences of protein promoter variants). The sequences of the additional components of the AAV constructs, except for sequences encoding the CasX protein (Table 21), are listed in Table 26.

TABLE 24
Sequences of protein promoter variants, construct IDs of AAV constructs that
comprise each respective protein promoter variant, and SEQ ID NOs for the
sequences of each protein promoter variant.

| Promoter | | Construct | SEQ ID |
|---|---|---|---|
| variant | Sequence | ID | NO: |
| UbC | GGCCTCCGCGCCGGGTTTTGGCGCCTCCCGCGGGCGCCCCC | 183 | 41030 |
| | CTCCTCACGGCGAGCGCTGCCACGTCAGACGAAGGGCGCAG | | |
| | CGAGCGTCCTGATCCTTCCGCCCGGACGCTCAGGACAGCGG | | |
| | CCCGCTGCTCATAAGACTCGGCCTTAGAACCCCAGTATCAG | | |
| | CAGAAGGACATTTTAGGACGGGACTTGGGTGACTCTAGGGC | | |
| | ACTGGTTTTCTTTCCAGAGAGCGGAACAGGCGAGGAAAAGT | | |
| | AGTCCCTTCTCGGCGATTCTGCGGAGGGATCTCCGTGGGGC | | |
| | GGTGAACGCCGATGATTATATAAGGACGCGCCGGGTGTGGC | | |
| | ACAGCTAGTTCCGTCGCAGCCGGGATTTGGGTCGCGGTTCT | | |
| | TGTTTGTGGATCGCTGTGATCGTCACTTGGT | | |
| Jet | GGGCGGAGTTAGGGCGGAGCCAATCAGCGTGCGCCGTTCCG | 191 | 41031 |
| | AAAGTTGCCTTTTATGGCTGGGCGGAGAATGGGCGGTGAAC | | |
| | GCCGATGATTATATAAGGACGCGCCGGGTGTGGCACAGCTA | | |
| | GTTCCGTCGCAGCCGGGATTTGGGTCGCGGTTCTTGTTTGT | | |
| U1a | AATGGAGGCGGTACTATGTAGATGAGAATTCAGGAGCAAAC | 177 | 41032 |
| | TGGGAAAAGCAACTGCTTCCAAATATTTGTGATTTTTACAG | | |
| | TGTAGTTTTGGAAAAACTCTTAGCCTACCAATTCTTCTAAG | | |
| | TGTTTTAAAATGTGGGAGCCAGTACACATGAAGTTATAGAG | | |
| | TGTTTTAATGAGGCTTAAATATTTACCGTAACTATGAAATG | | |
| | CTACGCATATCATGCTGTTCAGGCTCCGTGGCCACGCAACT | | |
| | CATACT | | |
| MeP426 | AGCTGAATGGGGTCCGCCTCTTTTCCCTGCCTAAACAGACA | 192 | 41033 |
| | GGAACTCCTGCCAATTGAGGGCGTCACCGCTAAGGCTCCGC | | |
| | CCCAGCCTGGGCTCCACAACCAATGAAGGGTAATCTCGACA | | |
| | AAGAGCAAGGGGTGGGGCGCGGGCGCGCAGGTGCAGCAGCA | | |
| | CACAGGCTGGTCGGGAGGGCGGGGCGCGACGTCTGCCGTGC | | |
| | GGGGTCCCGGCATCGGTTGCGCGC | | |
| miniCMV | GTAGGCGTGTACGGTGGGAGGTCTATATAAGCAGAGCT | 193 | 41034 |
| SFCp | GGATCCTACTGACGAGGGGCTTGTCCAAACAAGCCCGGGCA | 194 | 41035 |
| | TGCCTAAACATGCCCTGATGCAATCCTGACGTCGTGAAGCA | | |
| | ATGATTATGCAATTTGAGCATGTCCAGACTAGCCCTGACGG | | |
| | ATGACGCTTGACGCAATTCCTGAGGCAAGTCTGAGCTTGTT | | |
| | CAAACTTGTCTTGAAGAAATTATGACGGACTGACGTATGGT | | |
| | GCAATATTGGGGCAATGCTTGACGTTCGCGGTAGGCGTGTA | | |
| | CGGTGGGAGGTCTATATAAGCAGAGCTCGTTTAGTGAACCG | | |
| | TCAGATCGCCTGGAGACGCCATCCACGCTGTTTTGACCTCC | | |
| | ATAGAAGTCACCGGGACCGATCCAGC | | |
| miniSV40 | TGCATCTCAATTAGTCAGCAACCATAGTCCCGCCCCTAACT | 195 | 41036 |
| | CCGCCCATCCCGCCCCTAACTCCGCCCAGTTCCGCCCATTC | | |
| | TCCGCCCCATGGCTGACTAATTTTTTTTATTTATGCAGAGG | | |
| | CCGAGGCCGCCTCGGCCTCTGAGCTATTCCAGAAGTAGTGA | | |
| | GGAGGCTTTTTTGGAGGCCTAGGCTTTTGCAAA | | |
| pJB42CAT5 | CTGACAAATTCAGTATAAAAGCTTGGGGCTGGGGCCGAGCA | 196 | 41037 |
| | CTGGGGACTTTGAGGGTGGCCAGGCCAGCGTAGGAGGCCAG | | |
| | CGTAGGATCCTGCTGGGAGCGGGGAACTGAGGGAAGCGACG | | |
| | CCGAGAAAGCAGGCGTACCACGGAGGGAGAGAAAAGCTCCG | | |
| | GAAGCCCAGCAGCG | | |
| MLP | GGGGGGCTATAAAAGGGGGTGGGGGCGTTCGTCCTCACTCT | 197 | 41038 |
| CMV core | GTGATGCGGTTTTGGCAGTACATCAATGGGCGTGGATAGCG | 198 | 41039 |
| | GTTTGACTCACGGGGATTTCCAAGTCTCCACCCCATTGACG | | |
| | TCAATGGGAGTTTGTTTTGGCACCAAAATCAACGGGACTTT | | |
| | CCAAAATGTCGTAACAACTCCGCCCCATTGACGCAAATGGG | | |
| | CGGTAGGCGTGTACGGTGGGAGGTCTATATAAGCAGAGCT | | |
| EFS | TGGCTCCGGTGCCCGTCAGTGGGCAGAGCGCACATCGCCCA | ND | 41040 |
| | CAGTCCCCGAGAAGTTGGGGGGAGGGGTCGGCAATTGAACC | | |
| | GGTGCCTAGAGAAGGTGGCGCGGGGTAAACTGGGAAAGTGA | | |
| | TGTCGTGTACTGGCTCCGCCTTTTTCCCGAGGGTGGGGGAG | | |
| | AACCGTATATAAGTGCAGTAGTCGCCGTGAACGTTCTTTTT | | |
| | CGCAACGGGTTTGCCGCCAGAACACAGGT | | |
| miniEF1α | GGGCAGAGCGCACATCGCCCACAGTCCCCGAGAAGTTGGGG | ND | 41041 |
| | GGAGGGGTCGGCAATTGATCCGGTGCCTAGAGAAGGTGGCG | | |
| | CGGGGTAAACTGGGAAAGTGATGTCGTGTACTGGCTCCGCC | | |
| | TTTTTCCCGAGGGTGGGGGAGAACCGTATATAAGTGCAGTA | | |
| | GTCGCCGTGAACGTTCTTTTTCGCAACGGGTTTGCCGCCAG | | |
| | AACACAG | | |
| hRPL30 | CCCCGCAGCCATTCTAGCTAGCGGTACCAATAGCAACCGGC | ND | 41042 |
| | AGCTGCCCTCCGCTTTTGCTCCGCCCCTTCTGCTTGCGATC | | |
| | TGTTTCCGCTTCCGGTCCCGCAGTTCCGGCTCTGCCGTGAA | | |
| | GAGCTTTGCATTGTGGGAAGTCTTTCCTTTCTCGTTCCCCG | | |
| | GCCATCTTAGCGGCTGCTGTTGGTGAGTGGGCTCCTACCGA | | |
| | CCGAGGTTTAGGCAGCGCGGGGAGCTTTGCGGGTTGCCATT | | |
| | TGTAACTCCGGATCCTAAAATTCCTGTCCTGTTCTCTGTCT | | |
| | CTTCTAGGTTGGGGGCCGTCCCGCTCCTAAGGCAGGAA | | |
| hRPS18 | AGCCCCGGAACCTTCGCTGTTCTCTTACCTATGAACCTTAC | ND | 41043 |
| | GAACTGTAAAGAAAGGCGCACCGGAAGTTGTGGTACCCAAG | | |
| | CCATACTCTCATAAATCCAGCCAGGTCGCGCTGAAACAGTT | | |
| | TCCGGAAGCACTTCTCCTAGATCGCACCGCCTCTTCCTCCT | | |
| | GGAAGCTATATAATGATATCGCGTCACTTCCGCTCTCTCTT | | |
| | CCACAGGAGGCCTACACGCCGCCGCTTGTGCTGCAGCC | | |
| hRPL13a | ACAGCGCTGACCGCGGAGGTCCAACCGGAAGAATGTCCGGA | ND | 41044 |
| | TTGGACATTCGGAAGAGGGCCCGCCTTCCCTGGGGAATCTC | | |
| | TGCGCACGCGCAGAACGCTTCGACCAATGAAAACACAGGAA | | |
| | GCCGTCCGCGCAACCGCGTTGCGTCACTTCTGCCGCCCCTG | | |
| | TTTCAAGGGATAAGAAACCCTGCGACAAAACCTCCTCCTTT | | |
| | TCCAAGCGGCTGCCGAAG | | |

*ND = no description.

### Aav Production:

Suspension-adapted HEK293T cells, maintained in FreeStyle 293 media, were seeded in 20-30 mL of media at 1.5E6 cells/mL on the day of transfection. Endotoxin-free pAAV plasmids with the transgene flanked by ITR repeats were co-transfected with plasmids supplying the adenoviral helper genes for replication and AAV rep/cap genome using PEI Max (Polysciences) in serum-free Opti-MEM media. Cultures were supplemented with 10% CDM4HEK293 (HyClone) three hours post-transfection. Three days later, cultures were centrifuged to separate the supernatant from the cell pellet. The supernatant was mixed with 40% PEG 2.5M NaCl and incubated on ice to precipitate AAV viral particles. The cell pellet, containing majority of the AAV vectors, was resuspended in lysis media (0.15 M NaCl, 50 mM Tris HCl, 0.05% Tween, pH 8.5), sonicated on ice, and treated with Benzonase (250 U/μL, Novagen) for 30 minutes at 37° C. The PEG-treated supernatant was centrifuged to pellet the precipitated AAV, while the crude lysate was centrifuged to remove cell debris from the virus containing supernatant, before combining the collected virus for further clarification using a 0.45 μm filter. AAV lysates were purified using affinity chromatography (POROS CaptureSelect AAVX, ThermoFisher), and the eluate was buffer exchanged and concentrated in PBS+200 mM NaCl+0.001% Pluronic.

To determine the viral genome (vg) titer, 1 μL from crude lysate viruses was digested with DNase and ProtK, followed by quantitative PCR. 5 μL of digested virus was used in a 25 μL qPCR reaction composed of IDT primetime master mix and a set of primer and 6′FAM/Zen/IBFQ probe (IDT) designed to amplify a 62 bp-fragment located in the AAV2-ITR. An AAV ITR plasmid was used as reference standards to calculate the titer (vg/mL) of viral samples. The qPCR program was set up as: initial denaturation step at 95′C for 5 minutes, followed by 40 cycles of denaturation at 95′C for 1 minute, and annealing/extension at 60° C. for 1 minute.

Culturing hNPCs In Vitro:

Immortalized hNPCs were cultured in hNPC medium (DMEM/F12 with GlutaMax, 10 mM HEPES, 1×NEAA, 1× B-27 without vitamin A, 1× N2 supplemented growth factors hFGF and EGF, Pen/Strep, and 2-mercaptoethanol). Prior to testing, cells were lifted with TrypLE, gently resuspended to dissociate neurospheres, quenched with media, spun down, and resuspended in fresh media. Cells were counted and directly seeded at a density of ˜10,000 cells per well on a 96-well plate coated with PLF (poly-DL-ornithine hydrobromide, laminin, and fibronectin) 24 hours prior to AAV transduction.

AAV Transduction of hNPCs, Followed by HLA Immunostaining and Flow Cytometry:

˜7,000 cells/well of hNPCs were seeded on PLF-coated 96-well plates. 24 hours later, seeded cells were treated with AAVs expressing the CasX:gRNA system. All viral infection conditions were performed at least in duplicate, with normalized number of viral genomes (vg) among experimental vectors, in a series of three-fold serial dilution of MOI ranging from 1E4 to 1E6 vg/cell. Five days post-transduction, AAV-treated hNPCs were lifted with TrypLE. After cell dissociation, staining buffer (3% fetal bovine serum in dPBS) was used for quenching. The dissociated cells were transferred to a round-bottom 96-well plate, followed by centrifugation and resuspension of cell pellets with staining buffer. After another centrifugation, cell pellets were resuspended in staining buffer containing the antibody (BioLegend) that would detect the B2M-dependent HLA protein expressed on the cell surface. After HLA immunostaining, cells were stained with DAPI to label cell nuclei. HLA+ hNPCs were measured using the Attune NxT flow cytometer. Decreased or lack of HLA protein expression would indicate successful editing at the B2M locus in these hNPCs. A subset of transduced hNPCs were also lifted for genomic DNA extraction and editing analysis via next-generation sequencing (NGS).

### NGS Processing and Analysis:

Genomic DNA (gDNA) from harvested cells were extracted using the Zymo Quick-DNA Miniprep Plus kit following the manufacturer's instructions. Target amplicons were formed by amplifying regions of interest from 200 ng of extracted gDNA with a set of primers specific to the target locus, such as the human B2M locus. These gene-specific primers contain an additional sequence at the 5′ end to introduce an Illumina adapter and a 16-nucleotide unique molecule identifier. Amplified DNA products were purified with the Ampure XP DNA cleanup kit. Quality and quantification of the amplicon were assessed using a Fragment Analyzer DNA Analysis kit (Agilent, dsDNA 35-1500 bp). Amplicons were sequenced on the Illumina Miseq according to the manufacturer's instructions. Raw fastq files from sequencing were quality-controlled and processed using cutadapt v2.1, flash2 v2.2.00, and CRISPResso2 v2.0.29. Each sequence was quantified for containing an insertion or deletion (indel) relative to the reference sequence, in a window around the 3′ end of the spacer (30 bp window centered at −3 bp from 3′ end of spacer). CasX activity was quantified as the total percent of reads that contain insertions, substitutions, and/or deletions anywhere within this window for each sample.

Reprogramming of Induced Pluripotent Stem Cells (iPSCs):

Fibroblast cells from a patient were obtained from the Coriell Cell Repository. iPSCs were generated from these lines by episomal reprogramming and genetically engineered to ectopically express Neurogenin 2 (Neurog2) to accelerate neuronal differentiation. Three iPSC clones were selected for downstream experiments.

### Neuronal Cell Culture:

All neuronal cell culture was performed using N2B27-based media. To induce neuronal differentiation, iPSCs were plated in neuronal plating media (N2B27 base media with 1 μg/mL doxycycline, 200 μM L-ascorbic acid, 1 μM dibutyryl cAMP sodium salt, 10 μM CultureOne, 100 ng/ml of BDNF, 100 ng/ml of GDNF). iNs were dissociated, aliquoted, and frozen for long term storage after three days of differentiation (DIV3). DIV3 iNs were thawed and seeded on a 96-well plate at 30,000 cells per well. iNs were cultured for one week in plating media and thereafter, half-media changes were performed once every week using feeding media (N2B27 base media with 200 μM L-ascorbic acid, 1 μM dibutyryl cAMP sodium salt, 200 ng/ml of BDNF, 200 ng/ml of GDNF).

### AAV Transduction of iNs In Vitro:

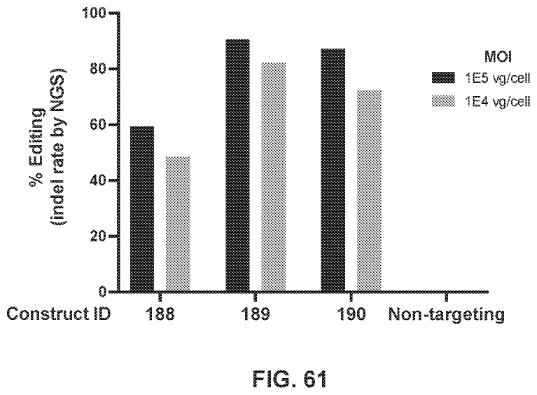

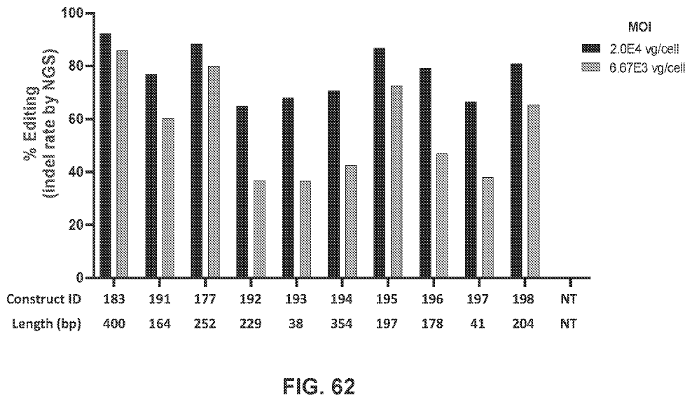

24 hours prior to transduction, ˜30,000-50,000 iNs per well were seeded on Matrigel-coated 96-well plates. AAVs expressing the CasX:gRNA system were then diluted in neuronal plating media and added to cells, with six wells per condition used as replicates. Cells were transduced at various MOIs (1E4 or 1E5 vg/cell for FIG. 61; 2E4 or 6.67E3 for FIG. 62). Seven days post-transduction, iNs were replenished using feeding media. 14 days post-transduction, cells were lifted using lysis buffer, 6-well replicates were pooled, and gDNA was harvested and prepared for editing analysis at either the human AAVS1 or B2M locus using NGS.

### Results:

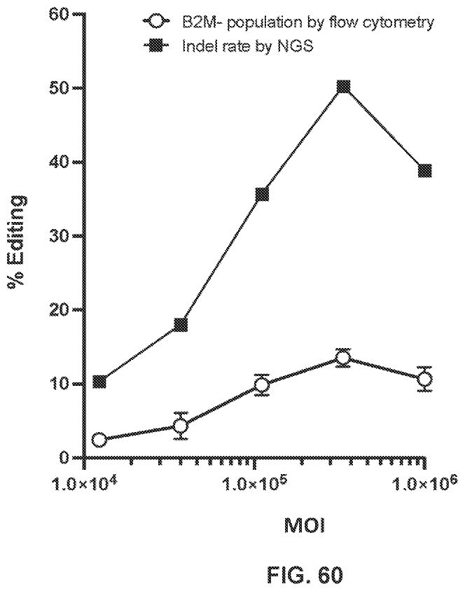

FIG. 60 shows the quantification of percent editing at the B2M locus measured via two different assessments (as indel rate quantified genotypically by NGS and as a phenotypic readout B2M-cell population detected by flow cytometry) in human NPCs five days post-transduction with AAVs at various MOIs. Efficient editing at the human B2M locus was observed, with the highest level of editing achieved at the MOI of ˜3E5: ˜50% indel rate and ˜13% of cells exhibiting the B2M protein knockout phenotype. FIG. 61 also illustrates efficient editing at the AAVS1 locus in human iNs, with construct ID 189 achieving ˜90% editing at the higher MOI of 1E5. As expected, no editing was observed at the AAVS1 locus with the non-targeting spacer.

FIG. 62 shows that robust editing at the B2M locus was achieved for several of the various protein promoters used to drive expression of CasX variant 491. Briefly, AAVs were generated with the indicated transgene constructs and transduced into human iNs at either an MOI of 2E4 or 6.67E3. AAV constructs 177 and 183 contained promoters that demonstrated the highest editing activity, with at least 80% efficiency at either MOI.

The results of these experiments demonstrate that CasX variant 491 and guide scaffold 235 with spacer targeting either the human B2M locus or the human AAVS1 locus can edit on-target efficiently when packaged and delivered in vitro via AAVs into human NPCs or iNs.

### Example 18: CpG-Depleted AAVs Demonstrate Effective CasX-Mediated Editing and Induce Less TLR9-Mediated Immune Response In Vitro

Pathogen-associated molecular patterns (PAMPs) such as unmethylated CpG motifs are small molecular motifs conserved within a class of microbes. They are recognized by toll-like receptors (TLRs) and other pattern recognition receptors in eukaryotes and often induce a non-specific immune activation. In the context of gene therapy, therapeutics containing PAMPs are often not as well-tolerated and are rapidly cleared from the patient given the strong immune response triggered, which ultimately leads to reduced therapeutic efficiency. CpG motifs are short single-stranded DNA sequences containing the dinucleotide CG. When these CpG motifs are unmethylated, they act as PAMPs and therefore potently stimulate the immune response.

Experiments were performed to deplete CpG motifs in the AAV construct encoding CasX variant 491, guide scaffold variant 235, and spacer 7.37 targeting the endogenous B2M locus (construct ID 183), and to demonstrate that CpG-depleted AAV vectors were able to edit effectively in vitro. Furthermore, experiments will be performed to assess the effects of CpG depletion on the activation of TLR9-mediated immune response in vitro. Individual elements of the AAV genome and their respective CpG-reduced versions are initially subjected to in vitro assessments of editing activity and immunogenicity to identify the optimal CpG-depleted sequences that yield potent editing but reduce undesired TLR9 activation, before being combined to generate an AAV genome with drastically reduced CpG presence for further evaluation.

### Materials and Methods:

### Generation of CpG-Depleted AAV Plasmids:

Nucleotide substitutions to replace native CpG motifs were designed in silico based on homologous nucleotide sequences from related species for the following elements: the murine U1a snRNA (small nuclear RNA) gene promoter, the human UbC (polyubiquitin C) gene promoter, the bGHpA (bovine growth hormone polyadenylation) sequence, and the human U6 promoter. The coding sequence for CasX 491 was codon optimized for CpG depletion, and the AAV2 ITRs were CpG-depleted as previously described (Pan X, Yue Y, Bofisi M. et al., 2021, Rational engineering of a functional CpG-free ITR for AAV gene therapy. Gene Ther. https://doi.org/10.1038/s41434-021-00296-0). All resulting sequences (Table 25) were ordered as gene fragments with the appropriate overhangs for cloning and isothermal assembly to replace individually the corresponding elements of the existing base AAV plasmid (construct ID 183). Spacer 7.37 (GGCCGAGAUGUCUCGCUCCG; SEQ ID NO: 379), which targets the endogenous gene beta-2-microglobulin (B2M), was used for the relevant experiments discussed in this example. Following isothermal assembly, AAV constructs were transformed into chemically competent E. coli cells (Stbl3s), which were plated on kanamycin LB-agar plates following recovery at 37° C. for 1 hour. Single colonies were picked for colony PCR and Sanger-sequenced. Sequence-validated constructs were midi-prepped for subsequent nucleofection and AAV vector production. The sequences of the additional components of the AAV constructs not depleted for CpG, except for sequences encoding CasX (Table 21), are listed in Table 26. Based on the demonstration of robust expression of CRISPR components and retention of editing activity, AAV constructs with the remaining unaltered components of Table 26 will be modified to deplete the CpG motifs and evaluated using the methods described in Example 17.

TABLE 25
Sequences of CpG-depleted AAV elements.

| CpG-depleted | | Construct | SEQ ID |
|---|---|---|---|
| element | Sequence | ID | NO: |
| CpG-depleted UbC | — | 184 | 41045 |
| promoter | | | |
| Strongly CpG- | — | 185 | 41046 |
| depleted UbC | | | |
| promoter | | | |
| CpG-free UbC | — | 186 | 41047 |
| promoter | | | |
| CpG-depleted Ula | — | 178 | 41048 |
| promoter | | | |
| CpG-free Ula | — | 179 | 41049 |
| promoter | | | |
| CpG-depleted U6 | — | 180 | 41050 |
| promoter | | | |
| CpG-free U6 | — | 181 | 41051 |
| promoter | | | |
| CpG-free cMycNLS- | | ND | 41052 |
| Stx491-cMycNLS | | | |
| CpG-free bGH-polyA | — | 182 | 41053 |
| sequence | | | |
| CpG-free 5′ITR | — | ND | 41054 |
| CpG-free 3′ITR | — | ND | 41055 |

Production of AAV vectors were performed as described earlier in Example 17.

Viral genome titer was determined as described earlier in Example 17.

Culturing Human Neural Progenitor Cells (hNPCs) In Vitro:

Immortalized hNPCs were cultured in hNPC medium (DMEM/F12 with GlutaMax, 10 mM HEPES, 1×NEAA, 1× B-27 without vitamin A, 1× N2 supplemented growth factors hTFGF and EGF, Pen/Strep, and 2-mercaptoethanol). Prior to testing, cells were lifted with TrypLE, gently resuspended to dissociate neurospheres, quenched with media, spun down, and resuspended in fresh media. Cells were counted and directly used for nucleofection or will be seeded at a density of ˜10,000 cells per well on a 96-well plate coated with PLF (p)oly-DL-ornithine hydrobromide, laminin, and fibronectin) 48 hours prior to AAV transduction.

Plasmid Nucleofection into Human Neural Progenitor Cells (hNPCs):

AAV plasmids encoding the CasX:gRNA system, with or without CpG depletion of the individual elements of the AAV genome, were nucleofected into hNPCs using the Lonza P3 Primary Cell 96-well Nucleofector Kit. Plasmids were diluted into two concentrations: 50 ng/μL and 25 ng/μL. 5 μL of DNA was mixed with 20 μL of 200,000 hNPCs in the Lonza P3 solution supplemented with 18% V/V P3 supplement. The combined solution was nucleofected using the Lonza 4D Nucleofector System following program EH-100. The nucleofected solution was subsequently quenched with the appropriate culture media and then divided into three wells of a 96-well plate coated with PLF. Seven days post-nucleofection, hNPCs were lifted for B2M protein expression analysis via HLA immunostaining followed by flow cytometry. Subsequently, stacking of individual CpG-depleted elements to create a combined AAV genome with substantial CpG depletion will be performed and similarly tested for editing assessment at the B2M locus in vitro.

### Editing Activity Assessment by HLA Immunostaining and Flow Cytometry:

Seven days after nucleofection, AAV-treated hNPCs were lifted with TrypLE. After cell dissociation, staining buffer (3% fetal bovine serum in dPBS) was used for quenching. The dissociated cells were transferred to a round-bottom 96-well plate, followed by centrifugation and resuspension of cell pellets with staining buffer. After another centrifugation, cell pellets were resuspended in staining buffer containing the antibody (BioLegend) that would detect the B2M-dependent HLA protein expressed on the cell surface. After HLA immunostaining, cells were stained with DAPI to label cell nuclei. HLA+ hNPCs were measured using the Attune NxT flow cytometer.

AAV Transduction of hNPCs In Vitro:

˜10,000 cells/well of hNPCs will be seeded on PLF-coated 96-well plates. 48 hours later, seeded cells will be treated with AAVs expressing the CasX:gRNA system, with or without CpG depletion of the individual elements of the AAV genome. All viral infection conditions will be performed at least in duplicate. 5-7 days post-transduction, hNPCs will be lifted for editing activity assessment via HLA immunostaining followed by flow cytometry as described. Subsequently, stacking of individual CpG-depleted elements to create a combined AAV genome with substantial CpG depletion will be performed and similarly tested for editing assessment at the B2M locus in vitro.

Use of human TLR9 reporter HEK293 cells (HEK-Blue™ hTLR9) for the in vitro immunogenicity assessment post-transduction with CpG-containing (CpG⁺) or CpG-depleted (CpG) AAVs:

The HEK-Blue™ hTLR9 line (InvivoGen) is derived from HEK293 cells, specifically designed for the study of TLR9-induced NF-κB signaling. These HEK-Blue™ hTLR9 cells overexpress the human TLR9 gene, as well as a SEAP (secreted embryonic alkaline phosphatase) reporter gene under the control of an NF-κB inducible promoter. SEAP levels in the cell culture medium supernatant, which can be quantified using colorimetric assays, report TLR9 activation.

For this experiment, 5,000 HEK-Blue™ hTLR9 cells will be plated in each well of a 96-well plate in DMEM medium with 10% FBS and Pen/Strep. The next day, seeded cells were transduced with CpG⁺ or CpG⁻ AAVs expressing the CasX:gRNA system. All viral infection conditions will be performed at least in duplicate, with normalized number of viral genomes (vg) among experimental vectors, in a series of three-fold serial dilution of MOI starting with the effective MOI of 1E6 vg/cell. Levels of secreted SEAP in the cell culture medium supernatant will be assessed using the HEK-Blue™ Detection kit at 1, 2, 3, and 4 days post-transduction following the manufacturer's instructions.

### Results:

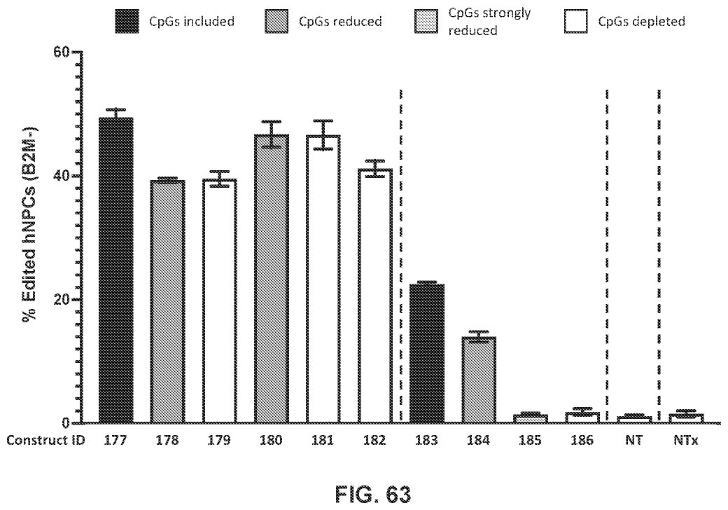

FIG. 63 shows the findings of an assay assessing the editing activity at the B2M locus in hNPCs nucleofected with CpG-containing (CpG⁺) or CpG-depleted (CpG) AAV vectors. Editing activity was measured as the percentage of hNPCs that were edited at the B2M locus, resulting in reduced/lack of B2M expression (B2M⁻) on the cell surface. FIG. 63 illustrates that reducing or depleting CpG motifs within the sequences of the U1a promoter (construct ID 178 and 179), Pol III U6 promoter (construct ID 180 and 181), or bGH poly(A) (construct ID 182) did not substantially decrease editing activity compared to the editing level achieved with the original CpG⁺ AAV construct (construct ID 177). Specifically, CpG⁻ U1a, CpG⁻ U6, or CpG⁻ bGH resulted in ˜80%, ˜94%, or ˜83% editing of the editing level attained with the base CpG⁺ AAV construct. However, reducing or depleting CpG motifs within the UbC promoter sequence (construct ID 184, 185, and 186) substantially diminished editing activity compared to the level seen with the base UbC construct (construct ID 183), highlighting context-dependent effects of CpG depletion on AAV editing activity and underscoring the importance to screen individual CpG-depleted AAV elements that would yield potent editing. These findings will be validated in experiments involving hNPC transduction with CpG⁺ or CpG⁻ AAVs. Individual CpG elements will also be stacked to generate a combined AAV genome with maximal CpG depletion, which will be evaluated for editing activity in vitro.

The experiments using HEK-Blue™ hTLR9 cells to assess TLR9-modulated immune response are expected to show reduced levels of secreted SEAP from cells treated with CpG AAVs in comparison to levels from cells treated with unmodified CpG⁺ AAVs. Reduced SEAP levels would indicate decreased TLR9-mediated immune activation.

### Example 19: In Vivo Administration of AAV Vectors with or without CpG-Depleted Genomes to Assess the Effects on Inflammatory Cytokine Production and CasX-Mediated Editing

Experiments will be performed to assess the effects of administering AAV vectors with or without CpG-depleted genomes in vivo. Briefly, AAV particles expressing the CasX:gRNA system (with or without CpG depletion) will be administered into C57BL/6J mice. In these experiments, the combined AAV genome with substantial CpG depletion will be used for assessment. After AAV administration, mice will be bled at various time points to collect blood samples. Production of inflammatory cytokines such as IL-1s, IL-6, IL-12, and TNF-α will be measured using ELISA.

### Materials and Methods:

### Generation of CpG-Depleted AAV Plasmids:

To assess the generation of transgene-specific T cells, a SIINFEKL peptide will be cloned into an AAV transgene plasmid on the N-terminus of the CasX protein. The SIINFEKL peptide is an ovalbumin-derived peptide that is well-characterized and has widely available reagents to probe for T cells specific for this peptide epitope. The nucleic acid sequence encoding this peptide will be cloned as an N-terminal fusion to CasX in an AAV construct with a ROSA26-targeting spacer.

Production of AAV vectors will be performed as described earlier in Example 17.

Viral genome titer will be determined as described earlier in Example 17.

### Measurement of Inflammatory Cytokines to Assess Humoral Immune Activation:

˜1E12 vg AAVs will be injected intravenously into C57BL/6J mice. Blood will be drawn daily from the tail vein or saphenous vein for seven days after AAV injection. Collected blood serum will be assessed for the levels of inflammatory cytokines, such as IL-1p, IL-6, IL-12, and TNF-α using commercially available ELISA kits according to the manufacturer's recommendations for murine blood samples (Abcam). Briefly, 50 μL of standard, control buffer, and sample will be loaded to the wells of an ELISA plate, pre-coated with a specific antibody to IL-10, IL-6, IL-12, or TNF-α, incubated at room temperature (RT) for two hours, washed, and incubated with horseradish peroxidase enzyme (HRP) for two hours at RT, followed by additional washes. Wells will be treated with TMB ELISA substrate and incubated for 30 minutes at RT in the dark, followed by quenching with H₂SO₄. Absorbance will be measured at 450 nm using a TECAN spectrophotometer with wavelength correction at 570 nm.

### Assessment of Transgene-Specific T Cell Populations:

Ten days after intravenous injection with AAVs, blood will be collected from mice, and T cells will be isolated using the EasySep™ Mouse T Cell Isolation kit. Isolated T cells will be incubated with the following: FITC mouse anti-human CD4 antibody (BD Biosciences), APC mouse anti-human CD8 antibody (BD Biosciences), and BV421 ovalbumin SIINFEKL MHC tetramer (Tetramer Shop). The percentage of CD4+ and CD8+ T cells specific to the SIINFEKL MHC tetramer will be quantified using flow cytometry. FITC, APC, and BV421 will be excited by the 488 nm, 561 nm, and 405 nm lasers and signal will be quantified with bandpass filters 440/50, 530/30 and 780/60 respectively.

### Quantification of CasX-Specific Antibodies:

Recombinantly produced and purified CasX variant 491 (methods to produce and purify are described in WO2020247882A1, incorporated by reference in its entirety) will be directly attached to the wells of a polystyrene 96-well plate by passive adsorption, using a carbonate/bicarbonate buffer at pH >9. Serum samples will then be assessed for the presence of CasX 491-specific antibodies using standard ELISA techniques employing commercially available HRP-conjugated secondary antibody kits according to the manufacturer's recommendations (Bethyl Laboratories). Absorbance will be measured at 450 nm using a TECAN spectrophotometer with wavelength correction at 570 nm.

### Quantification of AAV-Mediated Genome Editing at the ROSA26 Locus:

To demonstrate that CpG AAVs exhibit enhanced CasX editing activity relative to CpG⁺ AAVs in vivo, ˜1E12 AAV particles containing CasX protein 491 with gRNA targeting the ROSA26 locus will be administered intravenously via the facial vein of C57BL/6J neonates. Animals will be subsequently cared for following institutional animal use protocols at Scribe. Four weeks post-injection, mice will be euthanized, and the liver and/or muscle tissue will be harvested for gDNA extraction using the Zymo Quick DNA/RNA miniprep Kit following the manufacturer's instructions. Target amplicons will be amplified from 200 ng of extracted gDNA with a set of primers targeting the mouse ROSA26 locus of interest and processed as described earlier in Example 17.

### Results:

In vivo experiments measuring serum inflammatory cytokine levels are expected to show that CpG AAVs would significantly dampen production of inflammatory cytokines, such as EL-10, EL-6, E1-12, and TNF-α, thereby reducing immunogenicity and toxicity. In addition, CP AAVs are likely to cause less TLR9 activation leading to reduced expansion of T cells against the SHINFEKL peptide fused to CasX. Therefore, injections with CPU AAVs are expected to yield decreased levels of SIINFEKL-specific CD4+ and CD8+ T cells compared to levels from AAV constructs containing CpG elements.

Since CpG AAVs are likely to cause less humoral immune activation and non-specific inflammation, as well as less T-cell mediated immunity, titers of CasX-reactive antibodies are also expected to be reduced (i.e., lower ELISA signal quantifying CasX antibodies are anticipated).

Finally, editing capabilities of CpG AAVs will be assessed by harvesting muscle and/or liver tissue for genomic DNA extraction and subjected to NGS to determine editing levels at the ROSA26 locus. Enhanced CasX editing activity at the ROSA26 locus is anticipated with CpG AAVs, given their expected likelihood to elicit less humoral immune response in vivo.

TABLE 26
AAV constructs and component sequences*

| | | SEQ ID | | |
|---|---|---|---|---|
| Component | Name | NO: | Sequence | Constructs |
| 5′ ITR | AAV2 ITR | 40557 | ND | 1-174, 177-186, 188-198 |
| | CpG-free 5′ | 41777 | ND | ND |
| | ITR | | | |
| Enhancer + | CMV | 40645 | ND | 1-3, 7, 24-33, 44-52, 103-117 |
| core | | | | |
| promoter | | | | |
| | N/A | 40400 | ND | 1-3, 7, 24-33, 44-52, 64-71, 103- |
| | | | | 117, 156 |
| | Syn 1 | 40647 | ND | 65 |
| | NPC5 | 40648 | ND | 66 |
| | NPC7 | 40649 | ND | 67 |
| | NPC127 | 40650 | ND | 68 |
| | NPC190 | 40651 | ND | 69 |
| | NPC249 | 40652 | ND | 70 |
| | NPC286 | 40653 | ND | 71 |
| Protein | CMV | 40654 | ND | 1-3, 7, 24-33, 44-52, 103-117 |
| promoter | | | | |
| | UbC | 40655 | ND | 4, 34-37, 53, 78, 79-102, 119-155, |
| | | | | 157-174, 183, 188-190 |
| | EFS | 40372 | ND | 5, 38-40 |
| | CMV-s | 40657 | ND | 6, 41-43 |
| | CMVd1 | 40374 | ND | 8 |
| | CMVd2 | 40375 | ND | 9 |
| | miniCMV | 40376 | ND | 10 |
| | HSVTK | 40377 | ND | 11 |
| | miniTK | 40378 | ND | 12 |
| | miniIL2 | 40379 | ND | 13 |
| | GRP94 | 40664 | ND | 14 |
| | Supercore 1 | 40381 | ND | 15 |
| | Supercore 2 | 40382 | ND | 16 |
| | Supercore 3 | 40383 | ND | 17 |
| | Mecp2 | 40384 | ND | 18, 192 |
| | CMVmini | 40385 | ND | 19 |
| | CMVmini2 | 40386 | ND | 20 |
| | miniCMVIE | 40387 | ND | 21, 193 |
| | adML | 40388 | ND | 22 |
| | hepB | 40389 | ND | 23 |
| | RSV | 40390 | ND | 54 |
| | hSyn | 40675 | ND | 55 |
| | SV40 | 40676 | ND | 56 |
| | hPGK | 40677 | ND | 57 |
| | Jet | 40394 | ND | 58, 72-74, 191 |
| | Jet+UsP intron | 40679 | ND | 59, 75-77 |
| | hRLP30 | 40680 | ND | 60 |
| | hRPS18 | 40397 | ND | 61 |
| | CBA | 40682 | ND | 62 |
| | CBH | 40683 | ND | 63 |
| | CMV core | 40400 | ND | 64, 198 |
| | Ula | 41778 | ND | 177, 180, 181, 182, |
| | CpG-depleted | 41779 | ND | 178 |
| | Ula | | | |
| | CpG-free Ula | 41780 | ND | 179 |
| | CpG-depleted | 41781 | ND | 180 |
| | U6 | | | |
| | CpG-free U6 | 41782 | ND | 181 |
| | CpG- depleted | 41783 | ND | 184 |
| | UbC | | | |
| | Strongly CpG- | 41784 | ND | 185 |
| | depleted UbC | | | |
| | CpG-free UbC | 41785 | ND | 186 |
| | SFCp | 41786 | ND | 194 |
| | miniSV40 | 41787 | ND | 195 |
| | pJB42CAT5 | 41788 | ND | 196 |
| | MLP | 41789 | ND | 197 |
| | miniEFla | 41790 | ND | ND |
| | hRPL13a | 41791 | ND | ND |
| 5′ NLS aa | 1X SV40 NLS | 40685 | MAPKKKRKVSR | |
| sequence | | | | |
| | 4X SV40 NLS | 40686 | MAPKKKRKVGGSP | 121-123 |
| | | | KKKRKVGGSPKKK | |
| | | | RKVGGSPKKKRKV | |
| | | | SR | |
| | 1X Cmyc NLS | 40446 | PAAKRVKLDSR | 83, 84, 89-102, 124-131, 135-137, |
| | | | | 141-155, 157-174, 177-186, 188-198 |
| | 2X Cmyc NLS | 40447 | PAAKRVKLDGGSP | 127-129 |
| | | | AAKRVKLDSR | |
| | 4X Cmyc NLS | 40448 | PAAKRVKLDGGSP | 130, 131 |
| | | | AAKRVKLDGGSPA | |
| | | | AKRVKLDGGSPAA | |
| | | | KRVKLDSR | |
| | 6X Cmyc NLS | 40449 | PAAKRVKLDGGSP | 135-137, 142 |
| | | | AAKRVKLDGGSPA | |
| | | | AKRVKLDGGSPAA | |
| | | | KRVKLDGGSPAAK | |
| | | | RVKLDGGSPAAKR | |
| | | | VKLDSR | |
| | 1X | 40450 | KRPAATKKAGQAK | 132-134 |
| | Nucleoplasmin | | KKKSR | |
| | NLS | | | |
| | 2X | 40451 | KRPAATKKAGQAK | 138-140 |
| | Nucleoplasmin | | KKKGGSKRPAATK | |
| | NLS | | KAGQAKKKKSR | |
| | 1X Cmyc 1X | 40452 | PAAKRVKLDGGSP | ND |
| | SV40 NLS | | KKKRKVSR | |
| | 1X Cmyc 2′ 1X | 40453 | PAAKKKKLDGGSP | ND |
| | SV40 NLS | | KKKRKVSR | |
| | 1X Cmyc 2′ | 40454 | PAAKKKKLDSR | ND |
| | NLS | | | |
| | 3X Cmyc 2′ | 40455 | PAAKKKKLDGGSP | ND |
| | NLS | | AAKKKKLDGGSPA | |
| | | | AKKKKLDSR | |
| | 4X Cmyc 2′ | 40456 | PAAKKKKLDGGSP | ND |
| | NLS | | AAKKKKLDGGSPA | |
| | | | AKKKKLDGGSPAA | |
| | | | KKKKLDSR | |
| | 1X CPV NLS | 40457 | PAKRARRGYKCSR | ND |
| | IN | | | |
| | 2X CPV NLS | 40458 | PAKRARRGYKCGS | ND |
| | IN | | PAKRARRGYKCSR | |
| | 1X hBOVc | 40459 | PRRKREESR | ND |
| | NLS IN | | | |
| | 1X hBOVc | 40460 | PYRGRKESR | ND |
| | NLS 2N | | | |
| | 1X SIRT NLS | 40702 | PLRKRPRRSR | ND |
| | 2X SIRT NLS | 40703 | PLRKRPRRGSPLR | ND |
| | | | KRPRRSR | |
| | 1X Cmyc NLS | 40704 | PAAKRVKLDGGKR | ND |
| | 1X BPSV40 | | TADGSEFESPKKK | |
| | NLS GGS | | RKVGGS | |
| | 1X Cmyc NLS | 40705 | PAAKRVKLDGGKR | ND |
| | 1X BPSV40 | | TADGSEFESPKKK | |
| | NLS PPPPG | | RKVPPPPG | |
| | 1X Cmyc NLS | 40706 | PAAKRVKLDGGKR | ND |
| | 1X BPSV40 | | TADGSEFESPKKK | |
| | NLS px330 PG | | RKVGIHGVPAAPG | |
| | 1X Cmyc NLS | 40707 | PAAKRVKLDGGKR | ND |
| | 1X BPSV40 | | TADGSEFESPKKK | |
| | NLS (GGGS)2 | | RKVGGGSGGGSPG | |
| | PG | | | |
| | 1X Cmyc NLS | 40708 | PAAKRVKLDGGKR | ND |
| | 1X BPSV40 | | TADGSEFESPKKK | |
| | NLS | | RKVPGGGSGGGSP | |
| | P(GGGS)2 PG | | G | |
| | 1X Cmyc NLS | 40709 | PAAKRVKLDGGKR | ND |
| | 1X BPSV40 | | TADGSEFESPKKK | |
| | NLS alpha PG | | RKVAEAAAKEAAA | |
| | | | KEAAAKAPG | |
| | 1X Cmyc NLS | 40710 | PAAKRVKLDGGKR | ND |
| | 1X BPSV40 | | TADGSEFESPKKK | |
| | NLS PG | | RKVPG | |
| | 1X Cmyc GGS | 40711 | PAAKRVKLDGGSP | ND |
| | 1X SV40 GGS | | KKKRKVGGS | |
| | 1X Cmyc PPP | 40712 | PAAKRVKLDPPPP | ND |
| | 1X SV40 PG | | KKKRKVPG | |
| | 1X Cmyc PG | 40713 | PAAKRVKLDPG | ND |
| | 1X Cmyc | 40714 | PAAKRVKLDGGGS | ND |
| | (GGGS)3 | | GGGSGGGS | |
| | 1X Cmyc PPP | 40715 | PAAKRVKLDPPP | ND |
| | 1X Cmyc | 40716 | PAAKRVKLDGGGS | ND |
| | (GGGS)3 PPP | | GGGSGGGSPPP | |
| | 1X SV40 PPP | 40717 | PKKKRKVPPP | ND |
| | 1X SV40 GGS | 40718 | PKKKRKVGGS | ND |
| CasX | CpG-free | 41792 | ND | ND |
| | cMycNLS- | | | |
| | Stx491- | | | |
| | cMycNLS | | | |
| 3′ NLS aa | 1X SV40 NLS | 40461 | GSPKKKRKV | |
| sequence | | | | |
| | 4X SV40 NLS | 40462 | GSPKKKRKVGGSP | 149 |
| | | | KKKRKVGGSPKKK | |
| | | | RKVGGSPKKKRKV | |
| | 6S SV40 NLS | 40463 | GSPKKKRKVGGSP | ND |
| | | | KKKRKVGGSPKKK | |
| | | | RKVGGSPKKKRKV | |
| | | | GGSPKKKRKVGGS | |
| | | | PKKKRKV | |
| | 1X Cmyc NLS | 40464 | GSPAAKRVKLD | 141, 142, 150, 157-174, 177-186, |
| | | | | 188-198 |
| | 2X Cmyc NLS | 40465 | GSPAAKRVKLDGG | 151 |
| | | | SPAAKRVKLD | |
| | 4x Cmyc NLS | 40466 | GSPAAKRVKLDGG | ND |
| | | | SPAAKRVKLDGGS | |
| | | | PAAKRVKLDGGSP | |
| | | | AAKRVKLD | |
| | 6x Cmyc NLS | 40467 | GSPAAKRVKLDGG | 152 |
| | | | SPAAKRVKLDGGS | |
| | | | PAAKRVKLDGGSP | |
| | | | AAKRVKLDGGSPA | |
| | | | AKRVKLDGGSPAA | |
| | | | KRVKLD | |
| | 1X | 40468 | GSKRPAATKKAGQ | 119, 122, 125, 128, 130, 133, 136, |
| | Nucleoplasmin | | AKKKK | 139, 153 |
| | NLS | | | |
| | 2X | 40469 | KRPAATKKAGQAK | 120, 123, 126, 129, 131, 134, 137, |
| | Nucleoplasmin | | KKKGGSKRPAATK | 140, 154 |
| | NLS | | KAGQAKKKK | |
| | 2x | 40470 | GSPAAKRVKLGGS | 155 |
| | Nucleoplasmin | | PAAKRVKLGGSPK | |
| | 2x SV40 NLS | | KKRKVGGSPKKKR | |
| | | | KV | |
| | B19 NLS 1C | 40471 | GSKLGPRKATGRW | ND |
| | | | GS | |
| | BoV NLS 3C | 40472 | GSKRKGSPERGER | ND |
| | | | KRHWGS | |
| | 1X SV40 GS | 40473 | GSPKKKRKVGSGS | ND |
| | 1X | | KRPAATKKAGQAK | |
| | Nuceloplasmin | | KKKLE | |
| | NLS | | | |
| | GP vBPSV40 | 40474 | GPKRTADSQHSTP | 143 |
| | 12aa SV40 NLS | | PKTKRKVEFEPKK | |
| | | | KRKV | |
| | (GGGs)2vBPS | 40475 | GGGSGGGSKRTAD | ND |
| | V40 12aa SV40 | | SQHSTPPKTKRKV | |
| | | | EFEPKKKRKV | |
| | 3′alphahelix | 40476 | AEAAAKEAAAKEA | 144 |
| | vBPSV40 12aa | | AAKAKRTADSQHS | |
| | SV40 | | TPPKTKRKVEFEP | |
| | | | KKKRKV | |
| | GP SV40 GGS | 40477 | GPPKKKRKVGGSK | ND |
| | vBPSV40 12aa | | RTADSQHSTPPKT | |
| | SV40 | | KRKVEFEPKKKRK | |
| | | | V | |
| | GP alpha helix | 40478 | GPAEAAAKEAAAK | 145 |
| | Cmyc NLS | | EAAAKAPAAKRVK | |
| | | | LD | |
| | GP (GGGS)3 | 40479 | GPGGGSGGGSGGG | 146 |
| | Cmyc NLS | | SPAAKRVKLD | |
| | GP SV40 PPP | 40480 | GPPKKKRKVPPPP | 148 |
| | Cmyc NLS | | AAKRVKLD | |
| | GP Cmyc NLS | 40481 | GPPAAKRVKLD | 147 |
| | TGGGPGGGA | 40485 | TGGGPGGGAAAGS | ND |
| | AAGSGS- | | GSPKKKRKVGSGS | |
| | 1xSV40-GS- | | KRPAATKKAGQAK | |
| | Nuc | | KKKLE | |
| | TGGGPGGGA | 40487 | TGGGPGGGAAAGS | ND |
| | AAGSGS- | | GSPKKKRKVGSGS | |
| | 1xSV40-GS | | | |
| | PPPlinker | 40488 | PPPPKKKRKVPPP | ND |
| | 1xSV40 | | | |
| | PPPlinker | | | |
| | GGSlinker | 40489 | GGSPKKKRKVPPP | ND |
| | 1xSV40 | | | |
| | PPPlinker | | | |
| | PPPlinker | 40490 | PPPPKKKRKV | ND |
| | 1×SV40 | | | |
| | GGSlinker | 40491 | GGSPKKKRKV | ND |
| | 1xSV40 | | | |
| | GGSlinker | 40492 | GGSPKKKRKVGGS | ND |
| | 1xSV40 | | GGSGGS | |
| | (GGS)3linker | | | |
| | GGSlinker | 40493 | GGSPKKKRKVGGS | ND |
| | 2xSV40 | | PKKKRKV | |
| | (GGS)3linker | 40494 | GGSGGSGGSPKKK | ND |
| | 1xSV40 GGS | | RKVGGSPKKKRKV | |
| | 1XSV40 | | | |
| | PPP(GGGS)3li | 40749 | PPPGGGSGGGSGG | ND |
| | nker 1xCmyc | | GSPAAKRVKLD | |
| | PPPlinker | 40750 | PPPPAAKRVKLD | ND |
| | 1xCmyc | | | |
| | PPP(GGGS)3 | 40749 | PPPGGGSGGGSGG | ND |
| | linker 1xCmyc | | GSPAAKRVKLD | |
| PTRE | WPRE1 | 40752 | ND | 35, 38, 41, 72, 75, 78, 81, 83 |
| | WPRE2 | 40753 | ND | 36, 39, 42, 73, 76, 79, 82, 84, |
| | | | | 188-190 |
| | WPRE3 | 40433 | ND | 34, 37, 40, 43, 74, 77, 80 |
| PolyA | bGH | 40421 | ND | 1-23, 32, 33, 35-174, 177-181, 183- |
| signal | | | | 186, 188-198 |
| | hGH | 40756 | ND | 24 |
| | hGHshort | 40757 | ND | 25 |
| | HSVTK | 40424 | ND | 26 |
| | SynPolyA | 40425 | ND | 27 |
| | SV40 | 40426 | ND | 28 |
| | SV40short | 40427 | ND | 29 |
| | bglob | 40762 | ND | 30 |
| | bglobshort | 40429 | ND | 31 |
| | SV40polyA late | 40430 | ND | 34 |
| | CpG-free bGH | 41816 | ND | 182 |
| RNA | human U6 | 40401 | ND | 1-31, 34-84, 103-157, 177-179, |
| promoter | | | | 182-186, 188-198 |
| | H1 | 40402 | ND | 32, 158 |
| | 7SK | 40403 | ND | 33 |
| | hU6 variant 1 | 40404 | ND | 85, 89 |
| | hU6 variant 2 | 40405 | ND | 86 |
| | hU6 variant 3 | 40406 | ND | 87 |
| | hU6 variant 4 | 40407 | ND | 88 |
| | hU6 variant 5 | 40408 | ND | 90 |
| | hU6 variant 6 | 40409 | ND | 91 |
| | hU6 variant 7 | 40410 | ND | 92 |
| | hU6 variant 8 | 40411 | ND | 93 |
| | hU6 variant 9 | 40412 | ND | 94 |
| | hU6 variant 10 | 40413 | ND | 95 |
| | hU6 variant 1 1 | 40414 | ND | 96 |
| | hU6 variant 12 | 40415 | ND | 97 |
| | hU6 variant 13 | 40416 | ND | 98 |
| | hU6 variant 14 | 40417 | ND | 99 |
| | hU6 variant 15 | 40418 | ND | 100 |
| | hU6 variant 16 | 40419 | ND | 101 |
| | hU6 variant 17 | 40420 | ND | 102 |
| | H1 core | 41793 | ND | 159 |
| | H1 core + 7SK | 41794 | ND | 160 |
| | hybrid 1 | | | |
| | H1 core + 7SK | 41795 | ND | 161 |
| | hybrid 2 | | | |
| | H1 core + 7SK | 41796 | ND | 162 |
| | hybrid 3 | | | |
| | H1 core + 7SK | 41797 | ND | 163 |
| | hybrid 4 | | | |
| | H1 core + 7SK | 41798 | ND | 164 |
| | hybrid 5 | | | |
| | H1 core + 7SK | 41799 | ND | 165 |
| | hybrid 6 | | | |
| | H1 core + 7SK | 41800 | ND | 166 |
| | hybrid 7 | | | |
| | H1 core + 7SK | 41801 | ND | 167 |
| | hybrid 8 | | | |
| | H1 core + 7SK | 41802 | ND | 168 |
| | hybrid 9 | | | |
| | H1 core + U6 | 41803 | ND | 169 |
| | hybrid 1 | | | |
| | H1 core + U6 | 41804 | ND | 170 |
| | hybrid 2 | | | |
| | H1 core + 7SK | 41805 | ND | 171 |
| | + U6 hybrid 1 | | | |
| | H1 core + U6 | 41806 | ND | 172 |
| | hybrid 3 | | | |
| | H1 core + 7SK | 41807 | ND | 173 |
| | + U6 hybrid 2 | | | |
| | H1 core + 7SK | 41808 | ND | 174 |
| | + U6 hybrid 3 | | | |
| | hU6 isoform 2 | 41809 | ND | ND |
| | hU6 isoform 3 | 41810 | ND | ND |
| | hU6 isoform 4 | 41811 | ND | ND |
| | hU6 isoform 5 | 41812 | ND | ND |
| | hU6-CpG | 41813 | ND | 180 |
| | reduced | | | |
| | hU6-CpG | 41814 | ND | 181 |
| | depleted | | | |
| 3′ ITR | AAV2 ITR | 40576 | ND | 1-174, 177-186, 188-198 |
| | CpG-free 3′ | 41815 | ND | ND |
| | ITR | | | |

*Table lists component sequences except for sequences encoding nuclease, guide RNA, and linking peptides; ND = no description, sequence provided in sequence listing.

TABLE 27
Encoded targeting sequences incorporated
into AAV constructs

| Target | SEQ ID NOs of Targeting Sequences |
|---|---|
| Huntington (HTT) | 41056-41289 |
| Spacers | |
| PCSK9 Spacers | 41290-41319 |
| B2M Spacers | 41320-41477 |
| SOD1 Spacers | 41478-41571 |
| Rho Spacers | 41572-41611 |
| TRAC Spacers | 41612-41653 |
| DMD Spacers | 41654-41736 |
| BCL11A Spacers | 41737-41738 |
| C9Orf72 Spacers | 411739-41740 |
| PTBP1 Spacers | 41741-41776 |

### Example 20: Guide RNA Guide Scaffold Platform Evolution

Experiments were conducted to identify guide RNA guide scaffold variants that exhibit improved activity for double-stranded DNA (dsDNA) cleavage. In order to accomplish this, a large-scale library of scaffold variants was designed and tested in a pooled manner for functional knockout of a reporter gene in human cells. Scaffold variants leading to improved knockout were determined by sequencing the functional elements within the pool and subsequent computational analysis.

### Materials and Methods

### Library Design

### Assessment of RNA Secondary Structure Stability

RNAfold (v2.4.14) (Lorenz R, et al. ViennaRNA Package 2.0. Algorithms Mol Biol. 6:26 (2011)) was used to predict the secondary structure stability of RNA sequences, similar to what was done in Jarmoskaite I., et al. “A quantitative and predictive model for RNA binding by human pumilio proteins”, Mol Cell. 74(5):966 (2019). To assess the ΔΔG_BC value, the ensemble free energy (AG) of the unconstrained ensemble was calculated, then the ensemble free energy (AG) of the constrained ensemble was calculated. The ΔΔG_BC is the difference between the constrained and unconstrained AG values. A constraint string was used that reflects the base-pairing of the pseudoknot stem, scaffold stem, and extended stem, and requires the bases of the triplex to be unpaired.

### Calculation of Pseudoknot Stem Secondary Structure Stability

Pseudoknot structure stability was calculated for the entire stem-loop spanning positions 3-33, using the triplex loop sequence from guide scaffold 175. Further, a constraint string was generated that enforced pairing of the pseudoknot bases and unpairing of the bases in the triplex loop. Changes in stability could thus only be due to the differences in the sequence of the pseudoknot stem. For example, the pseudoknot sequence AAAACG_CGTTTT was turned into a stem-loop sequence by inserting the triplex loop sequence CUUUAUCUCAUUACUUUGA (SEQ ID NO: 41834), so that the final sequence would be AAAACGCUUUAUCUCAUUACUUUGACGTTTT (SEQ ID NO: 41835), and the constraint string was: ‘((((((xxxxxxxxxxxxxxxxxxx))))))’ (SEQ ID NO: 41836, where x=n).

### Molecular Biology

### Molecular Biology of Library Construction

The designed library of guide RNA scaffold variants was synthesized and obtained from Twist Biosciences, then amplified by PCR with primers specific to the library. These primers amplify additional sequence at the 5′ and 3′ ends of the library to introduce sequence recognition sites for the restriction enzyme SapI. PCR was performed with Q5 DNA Polymerase (New England Biolabs) and performed according to the manufacturer's instructions. Typical PCR conditions were: 10 ng of template library DNA, lx Q5 DNA Polymerase Buffer, 300 nM dNTPs, 300 nM each primer, 0.25 μl of Q5 DNA Polymerase in a 50 μl reaction. On a thermal cycler a typical program would be: cycle for 95° C. for 5 min; then 20 cycles of 98° C. for 15 s, 65° C. for 20 s, 72° C. for 1 min; with a final extension of 2 min at 72° C. Amplified DNA product was purified with DNA Clean and Concentrator kit (Zymo Research). This PCR amplicon, as well as plasmid pKB4, was then digested with the restriction enzyme SapI (New England Biolabs) and both were independently gel purified by agarose gel electrophoresis followed by gel extraction (Zymo) according to the manufacturer's instructions. Libraries were then ligated using T4 DNA Ligase (New England Biolabs), purified with DNA Clean and Concentrator kit (Zymo), and transformed into MegaX DH10B TiR Electrocomp Cells (ThermoFisher Scientific) all according to the manufacturer's instructions. Transformed libraries were recovered for one hour in SOC media, then grown overnight at 37° C. with shaking in 5 mL of 2xyt media. Plasmid DNA was then miniprepped from the cultures (QIAGEN). Plasmid DNA was then further cloned by digestion with restriction enzyme Esp3I (New England Biolabs), followed by ligation with annealed oligonucleotides possessing complementary single stranded DNA overhangs and the desired spacer sequence for targeting GFP. The oligonucleotides possessed 5′ phosphorylation modifications, and were annealed by heating to 95° C. for 1 min, followed by reduction of the temperature by two degrees per minutes until a final temperature of 25° C. was reached. Ligation was performed as a Golden Gate Assembly Reaction, where typical reaction conditions consisted of 1 μg of pre-digested plasmid library, 1 μM annealed oligonucleotides, 2 μL T4 DNA Ligase, 2 μL Esp3I, and 1× T4 DNA Ligase Buffer in a total volume of 40 μL water. The reaction was cycled 25 times between 37° C. for 3 minutes and 16° C. for 5 minutes. As above, the library was purified, transformed, grown overnight, and miniprepped. The resulting library of plasmids was then used for the production of lentivirus.

### Library Screening

### LV Production

Lentiviral particles were generated by transfecting LentiX HEK293T cells, seeded 24h prior, at a confluency of 70-90%. Plasmids containing the pooled library were introduced to a second generation lentiviral system containing the packaging and VSV-G envelope plasmids with polyethylenimine, in serum-free media. For particle production, media is changed 12 hours post-transfection, and viruses harvested at 36-48h post-transfection. Viral supernatant filtered using 0.45 μm PES membrane filters and diluted in cell culture media when appropriate, prior to addition to target cells.

72 hours post-filtration, aliquots of lentiviral supernatant were titered by TaqMan qPCR. Viral genomic RNA was isolated using a phenol-chloroform extraction (TRIzol), followed by alcohol precipitation. Quality and quantity of extraction was evaluated by nano-drop reading. Any residual plasmid DNA was then digested with DNase I just prior to cDNA production by ThermoFischer SuperScript IV Reverse Transcriptase. Viral cDNA was subject to serial dilutions through 1:1000 and combined with WPRE based primers and TaqMan Master Mix prior to qPCR by Bio-Rad CFX96. All sample dilutions are added in duplicate and averaged prior to titer calculations against a known, plasmid-based standard curve. Water is always measured as a negative control.

LV Screening (Transduction, Maintenance, Gating, Sorting, gDNA Isolation)

Target reporter cells are passed 24-48h prior to transduction to ensure cellular division occurs. At the point of transduction, the cells were trypsinized, counted, and diluted to appropriate density. Cells were resuspended with no treatment, library- or control-containing neat lentiviral supernatant at a low MOI (0.1-5, by viral genome) to minimize dual lentiviral integrations. The lentiviral-cellular mixtures were seeded at 40-60% confluency prior to incubation at 37° C., 5% CO₂. Cells were selected for successful transduction 48h post-transduction with puromycin at 1-3 μg/ml for 4-6 days followed by recovery in HEK or Fb medium.

Post-selection, cells were suspended in 4′,6-diamidino-2-phenylindole (DAPI) and phosphate-buffered saline (PBS). Cells were then filtered by Corning strainer-cap FACS tube (Prod. 352235) and sorted on the Sony MA900. Cells were sorted for knockdown of the fluorescent reporter, in addition to gating for single, live cells via standard methods. Sorted cells from the experiment were lysed, and the genome was extracted using a Zymo Quick-DNA Miniprep Plus following the manufacturer's protocol.

### Processing for Next Generation Sequencing (NGS)

Genomic DNA was amplified via PCR with primers specific to the guide RNA-encoding DNA, to form a target amplicon. These primers contain additional sequence at the 5′ ends to introduce Illumina read and 2 sequences. Typical PCR conditions would be: 2 μg of gDNA, lx Kapa Hifi buffer, 300 nM dNTPs, 300 nM each primer, 0.75 μl of Kapa Hifi Hotstart DNA polymerase in a 50 μl reaction. On a thermal cycler, cycle for 95° C. for 5 min; then 15 cycles of 98° C. for 15 s, 62° C. for 20 s, 72° C. for 1 min; with a final extension of 2 min at 72° C. Amplified DNA product is purified with Ampure XP DNA cleanup kit. A second PCR step was done with indexing adapters to allow multiplexing on the Illumina platform. 20 μl of the purified product from the previous step was combined with 1× Kapa GC buffer, 300 nM dNTPs, 200 nM each primer, 0.75 μl of Kapa Hifi Hotstart DNA polymerase in a 50 μl reaction. On a thermal cycler, cycle for 95° C. for 5 min; then 5-16 cycles of 98° C. for 15 s, 65° C. for 15 s, 72° C. for 30 s; with a final extension of 2 min at 72° C. Amplified DNA product is purified with Ampure XP DNA cleanup kit. Quality and quantification of the amplicon was assessed using a Fragment Analyzer DNA analyzer kit (Agilent, dsDNA 35-1500 bp). Amplicons were sequenced on the Illumina Miseq (v3, 150 cycles of single-end sequencing) according to the manufacturer's instructions. NGS analysis (sample processing and data analysis)

Reads were trimmed for adapter sequences with cutadapt (version 2.1), and the guide sequence (comprising the scaffold sequence and spacer sequence) was extracted for each read (also using cutadapt v 2.1 linked adapters to extract the sequence between the upstream and downstream amplicon sequence). Unique guide RNA sequences were counted, and then each scaffold sequence was compared to the list of designed sequences and to the sequence of guide scaffolds 174 (SEQ ID NO: 2238) and 175 (SEQ ID NO: 2239) to determine the identity of each.

Read counts for each unique guide RNA sequence were normalized for sequencing depth using mean normalization. Enrichment was calculated for each sequence by dividing the normalized read count in each GFP-sample by the normalized read count in the associated naive sample. For both selections (R2 and R4), the GFP- and naive populations were processed for NGS on three separate days, forming an enrichment value for each scaffold in triplicate. An overall enrichment score per scaffold was calculated after summing the read counts for the naive and GFP-samples across triplicates.

Two enrichment scores from different selections were combined by a weighted average of the individual log₂ enrichment scores, weighted by their relative representations within the naive population.

Error on the log 2 enrichment scores was estimated calculating a 95% confidence interval on the average enrichment score across triplicate samples. These errors are propagated when combining the enrichment values for the two separate selections.

### Results and Discussion

### Library Design, Ordering, and Cloning

A library of guide RNA variants was designed to both test variation to the RNA scaffold in an unbiased manner and in a targeted manner that focused on key modules within the RNA scaffold.

In the unbiased portion of the library, all single nucleotide substitutions, insertions, and deletions were designed to each residue of guide scaffolds 174 (SEQ ID NO: 2238) and 175 (SEQ ID NO: 2239) (˜2800 individual sequences). Double mutants were designed to specifically focus on areas that could possibly be interacting; thus if in the CryoEM structure (PDBid: 6NY2), two residues were involved in a canonical or non-canonical base pairing interaction, or two residues were predicted to pair in the lowest-energy structure predicted by RNAfold (v2.4.14), then the corresponding residues in guide scaffolds 174 and 174 were mutated (including all possible substitutions, insertions, and deletions of both residues). Adjacent residues to these ‘interacting’ residues were also mutated; however for these only substitutions of each of the two residues were included. In the final library, ˜27K sequences were designed with two mutations relative to guide scaffolds 174 or 175.

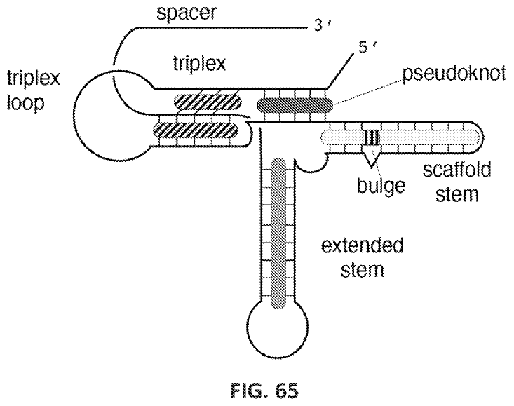

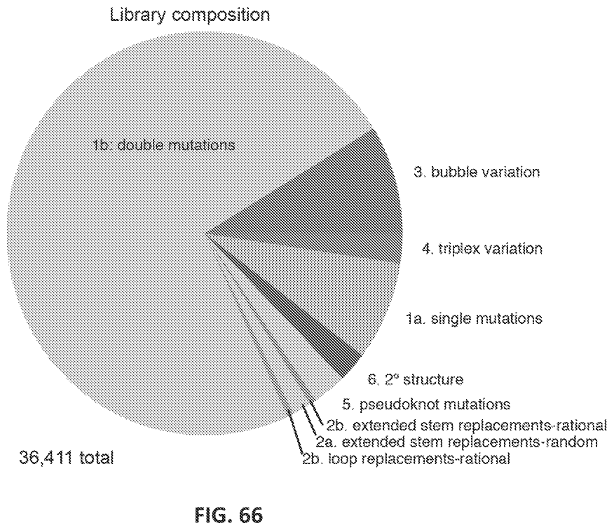

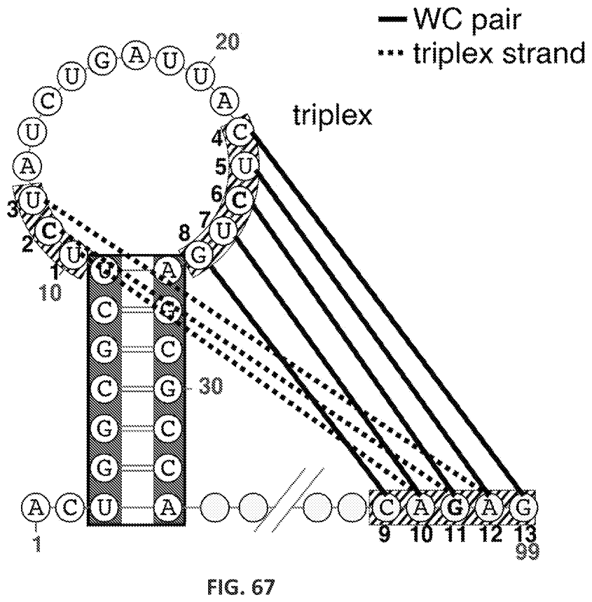

In the portion of the library devoted to specific mutagenesis of key regions of the RNA scaffold, modifications were designed to: the pseudoknot region, the triplex region, the scaffold bubble, and the extended stem (see FIG. 65 for region identification). In each of these targeted sections of the library, the entire domain was mutagenized in a hypothesis-driven manner (FIG. 66). As an example, for the triplex region, each of the base triplets that comprise the triplex was mutagenized to a different triplex-forming motif (see FIG. 67). This type of mutagenesis is distinct from that employed in the scaffold stem bubble, in which all possible substitutions of the bases surrounding the bubble were mutagenized (i.e., with up to 5 mutations relative to guide sequences 174 or 175). In contrast again, the 5 base-pairs comprising the pseudoknot stem were completely replaced with alternate Watson-Crick pairing sequence (up to 10 distinct bases mutagenized).

A final targeted section of the library was meant to optimize for sequences that were more likely to form secondary structures amenable to binding of the protein. In short, the secondary structure stability of a sequence was predicted under two conditions: 1) in the absence of any constraints, 2) constrained such that the key secondary structure elements such as pseudoknot stem, scaffold stem, and extended stem are formed (see Materials and Methods). Our hypothesis was that the difference in stability between these two conditions (called here ΔΔG_BC) would be minimal for sequences that are more amenable to protein binding, and thus we should search for sequences in which this difference is minimal).

The designed library was ordered from Twist (˜40K distinct sequences), and synthesized to include golden gate sites for cloning into a lentiviral plasmid backbone that also expressed the protein STX119 (see Materials and Methods). A spacer sequence targeting the GFP gene was cloned into the library vector, effectively creating single-guide RNAs from each RNA scaffold variant to target the GFP gene. The representation of the designed library variants was assessed with next generation sequencing (see Materials and Methods).

### Library Screening and Assessment

The plasmid library containing the guide RNA variants and a single CasX protein (version 119) was made into lentiviral particles (see Materials and Methods); particles were titered based on copy number of viral genomes using a qPCR assay (see Materials and Methods). A cell line stably expressing GFP was transduced with the lentiviral particle library at a low multiplicity of infection (MOI) to enforce that each cell integrated at most one library member. The cell pool was selected to retain only cells that had a genomic integration. Finally, the cell population was sorted for GFP expression, and a population of GFP negative cells was obtained. These GFP negative cells contained the library members that effectively targeted the CasX RNP to the GFP protein, causing an indel and subsequent loss of function.

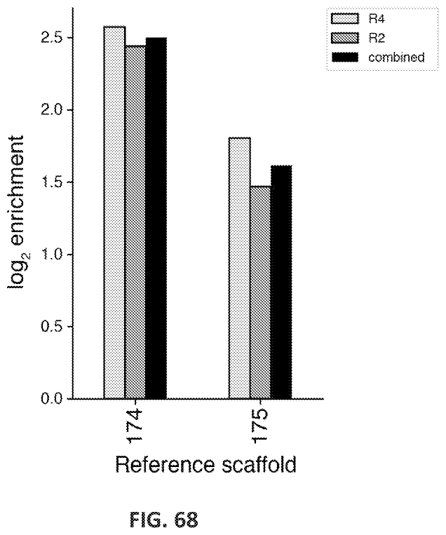

Genomic DNA from the unsorted cell population (“naive”) and the GFP negative population was processed to isolate the sequence of the guide RNA library members in each cell. To determine the representation of guide RNAs in the naive and GFP negative populations, next generation sequencing was performed. Enrichment scores were calculated for each library member by dividing the library member's representation in the GFP-population by its representation in the naive population: A high enrichment score indicates a library member that is much more frequent in the active, GFP negative population than in the starting pool, and thus is an active variant capable of effectively generating an indel within the GFP gene (enrichment value >1, log₂ enrichment >0). A low enrichment score indicates a library member that is depleted in the active GFP-population compared to the naive, and thus ineffective at forming an indel (enrichment value <1, log₂ enrichment <0). As a final statistic for comparison, the relative enrichment value was calculated as the enrichment of a library member (in the GFP negative vs naive population), divided by the enrichment of the reference scaffold sequence (in the GFP negative vs naive population). (In log space, these values are simply subtracted.) The enrichment values of the reference scaffold sequences are shown in FIG. 68).

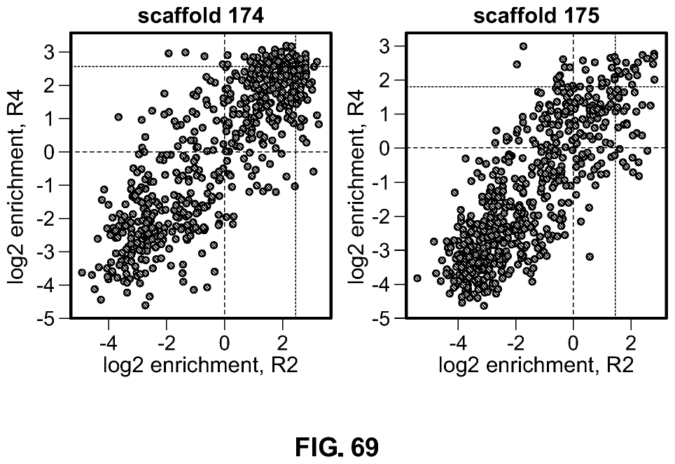

The screen was performed multiple times, with independent production of lentiviral particles, transduction of cells, selection and sorting to obtain naive and GFP negative populations, and sequencing to learn enrichment values of each library member. These screens were called R2 and R4, and largely reproduce the enrichment values obtained for single nucleotide variants on guide scaffolds 174 and 175 (FIG. 69). The screen was able to identify many possible combinations of mutations that were enriched in the functional GFP-population, and thus can lead to functional RNPs. In contrast, no guides that contained non-targeting spacers were enriched, confirming that enrichment is a selective cutoff (data not shown). The full set of mutations on guide scaffolds 174 and 175 that were enriched are given in Tables 28 and 29, respectively. These lists reveal the sequence diversity still capable of achieving targeted, functional RNPs.

### Single Nucleotide Mutations Indicate Mutable Regions of the Scaffold:

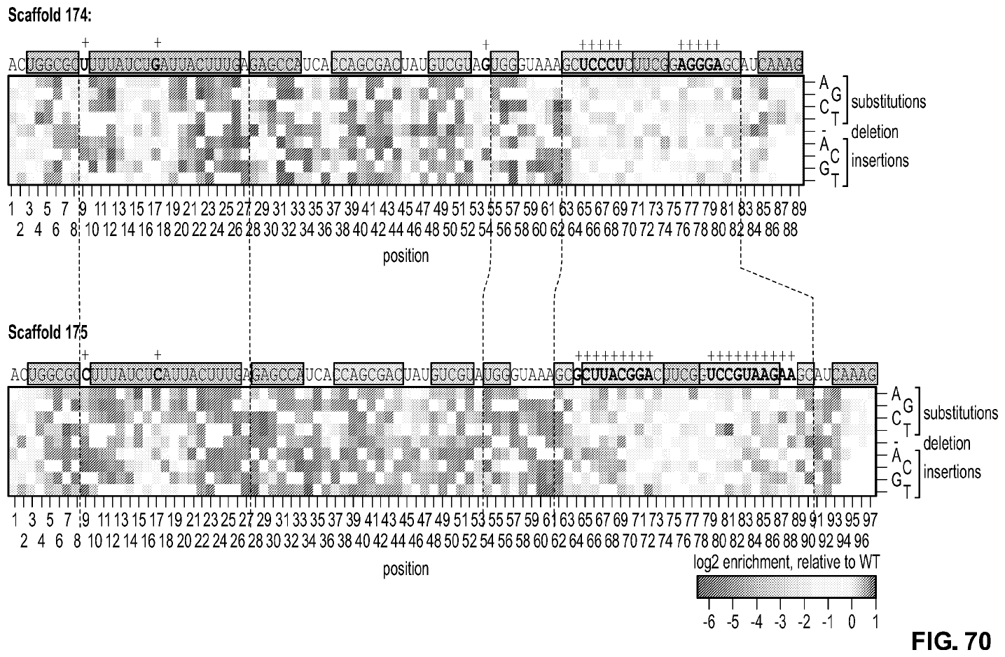

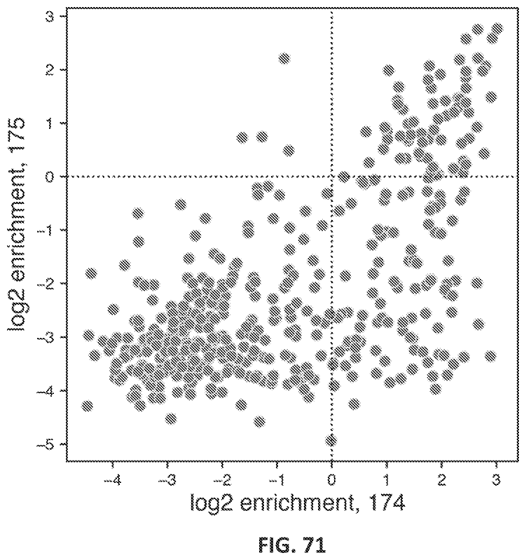

To determine scaffold mutations that lead to similar or improved activity relative to guide scaffolds 174 and 175, enrichment values of single nucleotide substitutions, insertions, or deletions were plotted (FIG. 70). Generally, single nucleotide changes on guide scaffold 174 were more tolerated than guide scaffold 175, perhaps reflecting higher activity of guide scaffold 174 in this context and thus a higher tolerance to mutations that dampen activity (FIG. 68 and FIG. 71). Single nucleotide mutations on 175 that were favorable were also favorable in the context of guide scaffold 174 in the vast majority of cases (FIG. 71), and thus the values for mutations on guide scaffold 175 were taken to be a more stringent readout of mutation effects. Key mutable areas were revealed by this analysis, as described in the following paragraphs:

The most notable feature was the extended stem, which showed similar enrichment values as the reference sequences for scaffolds 174 or 175, suggesting that the scaffold could tolerate changes in this region, similar to what has been seen in the past and would be predicted by structural analysis of the CasX RNP in which the extended stem is seen to have little contact with the protein.

The triplex loop was another area that showed high enrichment relative to the reference scaffold, especially when made in guide scaffold 175 (e.g., especially mutations to C15 or C17). Notably, the C17 position in 175 is already mutated to a G in scaffold 174, which is one of the two highly enriched mutations at this position to scaffold 175.

Changes to either member of the predicted pair in the pseudoknot stem between G7 and A29 were both highly enriched relative to the reference, especially in guide scaffold 175. This pair is a noncanonical G:A pairing in both guide scaffolds 174 and 175. The most strongly enriched mutation at these positions were in guide scaffold 175, converting A29 to a C or a T; the first of which would form a canonical Watson-Crick pairing (G7:C29), and the second of which would form a GU wobble pair (G7:U29), both of which may be expected to increase stability of the helix relative to the G:A pair. Converting the G7 to a T was also highly enriched, which would form a canonical pair (U7:A29) at this position. Clearly, these positions favor being more stably paired. In general, the 5′ end was mutable, with few changes leading to de-enrichment.

Finally, the insertion of a C at position 54 in guide scaffold 175 was highly enriched, whereas deletion of either the A or the inserted G at the analogous position in guide scaffold 174 both had similar enrichment values as the reference. Taken together, the guide scaffold may prefer having two nucleotides in this scaffold stem bubble, but it may not be a strong preference. These results are further examined in the sections below.

### Pseudoknot Stem Stability is Integral to Scaffold Activity

To further explore the effect of the pseudoknot stem on scaffold activity, the pseudoknot stem was modified in the following ways: (1) the base pairs within the stem were shuffled, such that each new pseudoknot has the same composition of base pairs, but in a different order within the stem; (2) the base pairs were completely replaced with random, WC-paired sequence. Two hundred ninety one (291) pseudoknot stems were tested. Analysis of the first set of sequences shows a strong preference for the G-A pair to be in the first position of the pseudoknot stem, relative to the other possible positions (positions 2-6; in the wildtype sequence it is in position 5;

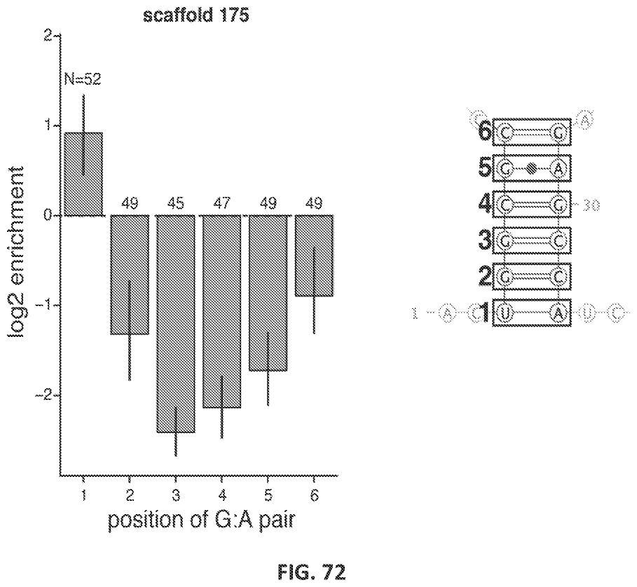

FIG. 72), while the results demonstrate that having a GA pair at each of the positions 2-6 in the pseudoknot stem is generally unfavorable, with low average enrichment. Having the G-A bases at position 1 likely stabilizes the pseudoknot stem by allowing the rest of the helix to form from stacking, Watson-Crick pairs only. This result further supports that the scaffold prefers a fully-paired pseudoknot stem.

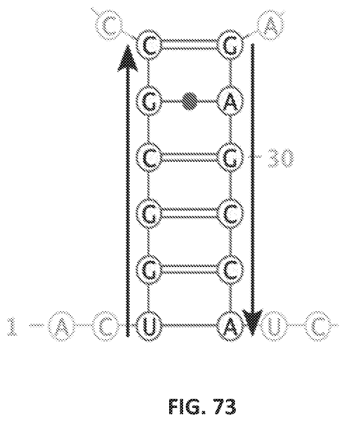

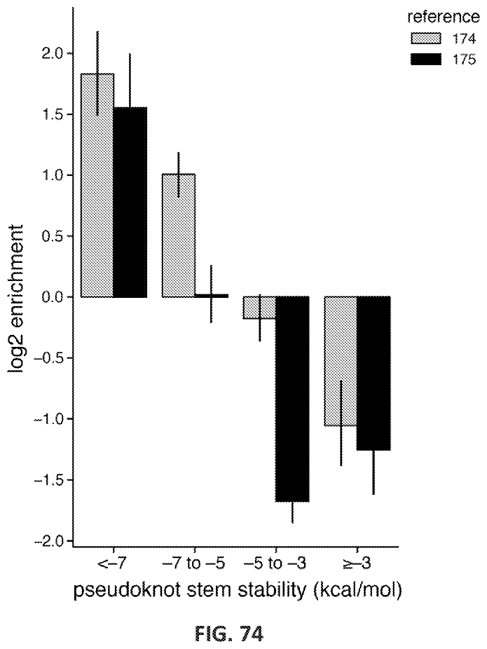

A substantial number of pseudoknot sequences had positive log 2 enrichment, suggesting that replacing this sequence with alternate base pairs was generally tolerated (pseudoknot structure in FIG. 73). To further test the hypothesis that a more stable helix in the pseudoknot stem would result in a more active scaffold, the secondary structure stability of each pseudoknot stem was calculated (Materials and Methods). A strong relationship was observed between pseudoknot stability and enrichment, and thus activity (FIG. 74: more active scaffold have stable pseudoknot stems), with guide scaffolds with stable pseudoknot stems (≤−7 kcal/mol) having high enrichment and guide scaffolds with destabilized pseudoknot stems (≥−3 kcal/mol) having very low enrichment.

### Double Mutations Indicate Mutable Regions of the Guide Scaffold:

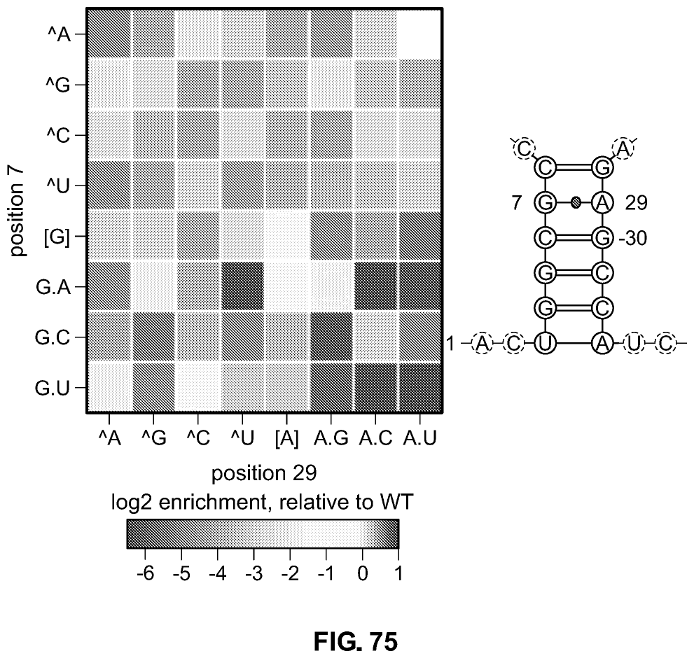

Double mutations to each reference guide scaffold were examined to further identify mutable regions within the scaffold, and potential mutations to improve scaffold activity. Focusing on just a single pair of positions-positions 7 and 29 which are predicted to form a noncanonical G:A pair in the pseudoknot stem and supports mutagenesis (see sections above)-we plot all 64 double mutations for this pair of positions (FIG. 75). Canonical pairs are favored at these two positions (e.g. substitution of a C at position 7 and a G at position 29 creates a G:C pair and is enriched; substitution of a C at position 7 and an insertion of a G at position 29 similarly creates a G:C pair, substitution of an A at position 7 and a U at position 29 creates an A:U pair). No pair of insertions was enriched, perhaps because inserting a canonical pair here is not sufficient to stabilize the helix given that the G:A pair is shifted up a position in the helix and not removed entirely. Surprisingly, several enriched double mutations did not form canonical pairs; e.g. substitutions of U at position 7 and C at position 29 (which forms a noncanonical U:C pair), substitutions of U at position 7 and U at position 29 (forming a U:U pair), as well as a few others (FIG. 75). It is possible that a purine:purine pair is substantially more disruptive to the helix than other noncanonical pairs. Indeed, substitution of an A at position 7 and G at position 29 again forms an A:G pair, which is not enriched at this position.

Enrichment values of double substitutions within each of the key structural elements of guide scaffold 175 were determined from heat maps in which each position could have up to three substitutions. It was determined that the scaffold stem was the least tolerant to mutation, suggesting a tightly constrained sequence in this region.

The results demonstrate substantial changes may be made to the guide scaffold that still result in functional gene knockout when utilized in an editing assay. In particular, the results demonstrate key positions that may be utilized to improve activity through modifications in the guide scaffold, including increased secondary structure stability of the pseudoknot stem within the scaffold.

TABLE 28
Guide 174 mutations and resulting relative enrichment

| Log₂ | |
|---|---|
| enrichment | Mutations on gRNA scaffold 174* (SEQ ID NO: 2238) |
| 3.25 to 3.5 | G79A, A80G; T34A, G78T; G7T, G75A; G78A, A80T; \hat{ }C2, A33T; \hat{ }A1, C68T; |
| | TG3CT, CGC6TAG, GAG28CTA, CA32AG; TG3CA, GC7AA, GA28TT, CA32TG; |
| | \hat{ }C4, C6G, T12_, G17C, GAG28CCC, C32G, A80C; T9C, T14A, T71A, C73A; |
| | C70A, G77T |
| 3.0 to 3.25 | A29T, G78T; T9C, G17C, A27T, G79_; C2G, A21G; \hat{ }A81, C81; T71A, C73A; |
| | T14C, T16G; \hat{ }T64, \hat{ }G81; T9C, G17C, \hat{ }TG65; C2G, T16A; |
| | G7C, TC14AT, G17A, T34A; G75A, G77A; G7C, A21T; T-.3.CA, GC.7.-T, G28_, |
| | -A.33.TG, \hat{ }T84; T65C, C82T; |
| | GCTCCC63_\hat{ }AATGAAAA70, \hat{ }TTTTCATT76, GGGAGC77_; \hat{ }C2, G7A, A27T; |
| | T9C, G17C, C67G; \hat{ }A78, \hat{ }T78; T3C, GCG5AGA, AGC29GCT, A33G; |
| | T9C, G17C, G78C; T3C, GC5TG, AGC29CAA, A33G; T9A, \hat{ }T68, G77A; |
| | G7A, T9G; T65A, \hat{ }G77; \hat{ }G70, \hat{ }C75; C2T, G79C; \hat{ }C66, G78A; A29C, G75A; |
| | C15A, A60G; C67G, \hat{ }A78; T14C, G17T, G40A, A76G; T34A, CT64TC; |
| | \hat{ }A69, T69A; T45G, G79T; T69C, \hat{ }C76; C2A, G54C; A13C, C15A, G74C; |
| | C70G, \hat{ }A75; A76G, G77C; C67T, G78C; TG3CC, A29C, CA32GG; \hat{ }T7, A29C; |
| | C2A, T34A; \hat{ }A66, \hat{ }A66; C66T, A80C; \hat{ }G17; \hat{ }C76, \hat{ }A76; A29C; |
| | C15G, C67G, T72G; \hat{ }T70, \hat{ }A70; C15G, T16G; C64T, C66A; T69G, G74C; |
| | \hat{ }A3, G74C; \hat{ }T65, \hat{ }T80 |
| 2.9 to 3.0 | A29C, A33T; C64T, G78A; \hat{ }C64, A80T; \hat{ }A74; T65A, \hat{ }A80; \hat{ }T69, G75T; |
| | \hat{ }C79, \hat{ }A79; A29G, T59A; T69G, G75C, G78A; \hat{ }G70, \hat{ }A70; |
| | G7A, TC14CG, G17A, C64T; \hat{ }T69, \hat{ }A76; T9C, G17C, C68T, T72C; \hat{ }T69, A76G; |
| | A33T, C66G; C66T, C67G; TTC71ACA, \hat{ }GGATGT75; A13G, T14A; |
| | T69A, G74C; G74T, A76G; G77C, G78A; A27C, T84G; C2_C66G; T71C, G75C; |
| | TC14AG, G78A; T3G, A33T; T9C, G28A; \hat{ }A1, C2T; C68T, T72C; |
| | TGGC3CCAG, C8A, GA26_, --A.33.TGG; C64T, C66G; \hat{ }A67, C67G; |
| | C68T, G74A, G77C; G7T; C2T, G78T; C68_G77T; T25C, A29C; \hat{ }A78, G78A; |
| | \hat{ }C78, G78C; G7C, A60G; T34A, T45A; T3_G7A, \hat{ }A9, \hat{ }T28, A29G, A33_; |
| | \hat{ }CAG70, T72G, G--.74.AGT; A27G, A29C; T9C, G17C, T47C; \hat{ }T19; \hat{ }A65, \hat{ }T65; |
| | C67G, C68T |
| 2.8 to 2.9 | T3C, G5T, C8G, GA28CC, CCA31AAG; T69C, A76G; C66T, A80T; \hat{ }G13; |
| | C2_T65G; G7C, T9G; T9C, G17C, TT71AC; C6G, A29T; \hat{ }C66, \hat{ }C79; |
| | C70A, A76T; T3A, CG6AC, AG29GT, A33G; \hat{ }T7, T12_; \hat{ }T69, \hat{ }A76, A88C; |
| | C35G, G58C; \hat{ }A79, \hat{ }T79; T16_C67T, G79_; G7T, T9A; A29T, C37T, C66G, \hat{ }G77; |
| | C2_, G81A; C15G, T34G; T3_\hat{ }T9, \hat{ }C28, A29C, C32_; \hat{ }T76, \hat{ }A76; G7C, A27T; |
| | C2_G79C; TGGC3ACAG, GA26_, --A.33.TGT; \hat{ }G65, \hat{ }G77; |
| | \hat{ }AC1, GC5_C8T, GA26_G30A, \hat{ }GT34; T9C, G17C, C66T, A80T; T71G, T72G; |
| | G4C, CT8GC, G17C, GA28AC, C32G, T69C, G75C; C41A, G51T; \hat{ }T78; |
| | T9C, G17C, T65A, A80C; AG29CA, C82G; T9G, C82T; T45A, T47C; C2T, T3A; |
| | T65A, A80G; C2G, G4A, C32T; G7C, T59G; T9C, T14G; C2G, A29C, T52A; |
| | T9C, G17C, -A.53.CC; T9C, T69_A76_; C68A, G75C; A1G, A33T; |
| | T3_, \hat{ }T9, G28_\hat{ }G32; \hat{ }G70, G75C; \hat{ }C54, G54C; \hat{ }T79, G79A; G17C, C70T, A76G; |
| | G77A; \hat{ }T69, A76C; T65A, \hat{ }C80; \hat{ }A66, G79_; T9G, \hat{ }G85; \hat{ }TGGAAGAT63, C--- |
| | 66.TCGG, C68A, GGAGGGAG74_\hat{ }A83; \hat{ }T2; G7A, A29C; \hat{ }A69, \hat{ }C76; |
| | C6A, A29C; C2_, T9C, G17C, GA79TG |
| 2.7 to 2.8 | T34A, \hat{ }T37; A36T, T65C; C2_, T69G; C73A, G74C; G17_; \hat{ }G65, \hat{ }A65; |
| | \hat{ }T67, C67T; C2G, A29T; T9C, G17C, \hat{ }C66, \hat{ }G74; C70A, T71C; T14A, C15T; |
| | G4C, C32G, G78C; T9C, G17C, T34A; \hat{ }A66, \hat{ }C79; AGT53GTG; G79_; |
| | T9G, T14A; \hat{ }C64, \hat{ }C80; T65C, \hat{ }G66; \hat{ }GT1, G7A, T9C; A60T, G78T; |
| | T9C, G17C, C67A, G79A; TC65CG, A80G; T14C, T16C; T3_; |
| | \hat{ }CGAAC70, T71A, G74C; G7T, C8G; T3A, GC7CG, GA28CG, A33T; C66T, G78T; |
| | A1G, T9C, G17C; T69C, C70A; C70T, T72G; T69C, T71G, A80T; T16G, A29C; |
| | T11G, A29C; G17A, \hat{ }TA75, A88C; G7T, G40A, A61G; \hat{ }AC81, A88C; \hat{ }A71; |
| | G5C, C8G, GA28AC, C31G, C73T; G74T, A76C; \hat{ }T68, \hat{ }A76; C2_, C70A; |
| | T9C, G28T; G28T, A29C; \hat{ }C29; A29C, GA75AC; \hat{ }T52, G54C; G7A, T9C, G17C; |
| | T9C, G17C, G79A; --A.29.CAC; \hat{ }A68, G77A; \hat{ }T69; G7C, T9C; A80C, C82T; |
| | \hat{ }C75, G75T; T14A, A29C; T72C, C73A; T9C, G17C, C66A, G79_; C2_, A33T; |
| | \hat{ }T2, C64T; \hat{ }AT79, A88C; C66G, A80C; \hat{ }A67, \hat{ }T78; \hat{ }G67, G78A; \hat{ }A76; |
| | A21G, \hat{ }C66, \hat{ }T77; C2A, A36G, T69A; G63T, T71C; T9C, G17C, -G.77.CT; |
| | \hat{ }T2, T34A; C68T, \hat{ }C77; T9C, G17C, T72C; T69A, C70T; CT15TA, A18T; |
| | TGG3ACA, C8G, GA28CC, CCA31TGT; T9C, A29C; C6G, G30C; -T.3.AA, C67G; |
| | C73_; \hat{ }G68, \hat{ }A76; T69C, \hat{ }A76; A80G; T69C, A76_; \hat{ }G68, \hat{ }T77 |
| 2.6 to 2.7 | T9A, A29C; A76G; T9C, G17C, AG76CC; T9C, \hat{ }A13; \hat{ }A67, \hat{ }T78, A88C; |
| | C70A, T72A; C66G, \hat{ }T79; \hat{ }T64, C64T; \hat{ }A70, C70G; \hat{ }G65, A80C; |
| | T9C, G17C, C66T, G78C; C2_, T9G; T69_, A76T; T3A, G7A, A29T, A33G; |
| | T45G, C68A; \hat{ }T65, \hat{ }T80, A88C; C66G; C64T, T71G; C2G, G54T; A1G, T3A; |
| | \hat{ }G70, G75T; T65A, \hat{ }T80; -T.3.AC, GC.7.A-, |
| | GAG28TGC, CA32GG, T72A, \hat{ }G74, A76G; A21C; T69G, \hat{ }A76; C68G, C70A; |
| | C67T, A80T; \hat{ }A70, \hat{ }C75; T9A, T14C; T3A, CGC6TCT, GAG28AGA, A33T; |
| | G54_; C68T; \hat{ }T65, G79C; C2_; C67G, G79T; CT2TG, G7A, G77C; T71G, G74A; |
| | C66T, G81A; A29T; --A.29.CAT, A88C; T69_\hat{ }C76; T9C, G17C, \hat{ }G68; |
| | \hat{ }A69, T69C; A29C, G30T; T69_; G17C; \hat{ }A67, G78C; T65A; \hat{ }G79, \hat{ }T79; |
| | A76G, G77T; \hat{ }GC1, A88C; A27T, A29C; \hat{ }CA79, A88C; T69_, G75T; |
| | C38G, \hat{ }C56, G77A; C68T, G77C; A29C, AG39GT, T52C; G79T, A80T; |
| | G7T, A61T; T16C; \hat{ }A13; G7C, C15G; G5C, C8G, GA28AC, C31G; C2_G77C; |
| | A29C, T52A; G75C, A76G; T9C, G17C, \hat{ }C76; C8G, A29C; |
| | TGG3GTC, C8G, GA28AC, CCA31GAC; |
| | C64_\hat{ }GTG67, C68A, G77T, \hat{ }CAC79, G81_; \hat{ }T68, G79_; \hat{ }A70, C70A; |
| | T65A, AG76GA; \hat{ }C70, \hat{ }C70; C68G, G77T; C6T, A29T; \hat{ }T81; \hat{ }G67, \hat{ }A67; |
| | TGG3GCA, C8A, A29C, CCA31TGC; G7A, A27C, A29G, A80G; G78A; |
| | T52G, G54C; T9C, G17C, T65A, C67T; A1C, \hat{ }C64, \hat{ }T81; \hat{ }T80, A80C; |
| | C67A, C73T; C73T, A80C; C67A, T69C; G7A, A76T, A80G; C2_, C15G; |
| | T69C, G77T; CT2_, G79T; G7C, \hat{ }G28; \hat{ }C79; \hat{ }A80; \hat{ }G1, \hat{ }C1; \hat{ }G65, A80T; |
| | G7T, A29_; -T.3.AC, GC.7.A-, GAG28TGC, CA32GG, T65A; |
| | T9C, \hat{ }T14, G17C, \hat{ }C29; A29T, T69C; T9C, A29G; C64T, T65C; |
| | \hat{ }TG70, T71A, C73_, G75T; T65G, C66T; T59C, \hat{ }C66; T72A, G74A; C2T, T72C; |
| | T71C, A76G; T65G, A80T; TG3_\hat{ }TG7, GA26_, \hat{ }AG33 |
| 2.5 to 2.6 | T9C, G17C, \hat{ }G81; --A.29.CAT; C68T, A76G; A29C, G79A; G17C, C67G, C70T; |
| | \hat{ }G66, C66G; A29T, G63A, C66A; G28C, A29C; T3G, C67A; T69C, T71C; |
| | T3A, GC7CA, GA28TG, A33G; C70G, G74A; \hat{ }C2, G4C, C8_\hat{ }CGC28, CCA31_; |
| | C2_, C68T; C66A, A80T; T3A, G5C, GC7AA, GA28TT, C31G, A33T; |
| | T9C, G17C, T72G; T9C, G17C, A29C; \hat{ }C70, G75T; C66T; C66T, G78A; |
| | A36T, G54C, C68T; \hat{ }G9, A29T; A76C; T69C, G77C; \hat{ }A77, \hat{ }G77; T71G, G74C; |
| | C67T; C73G; T71G, AG76GA; \hat{ }C64, T65C; T3G, C68A; G74C; C67T, T69A; |
| | \hat{ }A69; \hat{ }A66, C66T; T71C; T14G, T16C; T9C, G79T; T65C; \hat{ }C15, G17C; |
| | \hat{ }T65, \hat{ }C79; C70G, T71G; G74C, G75T; C2_, C68G; G7T, A27G; \hat{ }CA76, A88C; |
| | \hat{ }T65, \hat{ }A65; T9C, G17C, T45A; A18C, \hat{ }A66; A80C; G7C, TC14CT, G17A; |
| | TG3GC, G7A, A29T, CA32GC; T16G, A29C, G63T, T71C; C2A, G54T, T71C; |
| | \hat{ }T8, A29C; T9C, TG16GC; C70T, G77T; G75T, A76G; T69A; T16A, A18G; |
| | G77A, G78C; A1_, T59C; T14G, T16G; \hat{ }A60, G81A; A29G, A83G; |
| | T34A, GA79TC; T69C, G75A; G7T, T59A; G7T, C82G; A36T, G81T; C2_, G81T; |
| | T14C, T72_; --A.29.CAC, A88C; TGG3_, \hat{ }AAG9, GA28CT, C31G, A33G; |
| | G17C, A18G; C66G, G77A; \hat{ }C5, C6T, C8_, G28C, GC30CG; C82T; G54A, \hat{ }G56; |
| | C2_, C66T; G17C, A18C; G17C, G54_; G28A, T65C; C6T, A29C; G7A, T9C, \hat{ }T79; |
| | T9C, GA17CT; G74A, G75T; C68A, C70G; G42C, C50G; \hat{ }C70, \hat{ }C75; \hat{ }T66, \hat{ }C66; |
| | T3C, CGC6GCT, G28_, \hat{ }A32, A33G; C73A, G74A; |
| | TG3AC, C6A, AG29CT, CA32GG; C67A, G79A; A76_; C73G, G74T; |
| | TG3CA, GC7AG, GA28CG, CA32TG; T9C, T14C, T71A, C73A; G81C; |
| | A1G, T16A; T69A, \hat{ }G74; C68_; C2A, A60C; T9C, G54T; T14C, C15G; |
| | \hat{ }G66, \hat{ }G66; T16C, A18G; \hat{ }G68, G77C; A29T, -G.78.CC; G7T, \hat{ }T61; CT2_, T72G; |
| | A1G; T65C, C66A; G7C, T34A; \hat{ }C35, T59G; \hat{ }AG77, A88C; \hat{ }TG67, A88C; |
| 2.4 to 2.5 | G54C, T59A; T69G, G75C; C68A, A76G; \hat{ }AT65, A88C; C68T, G77T; G7T, A29C; |
| | T65A, T71A, G74A; T16A; \hat{ }C65, \hat{ }A65; \hat{ }T67, G79_; \hat{ }G71; \hat{ }C18; \hat{ }C29, A29T; |
| | G79A; T69G, T71A; T71C, T72C; C2_, T3_; \hat{ }T67, G78T; |
| | CTCCCTCT64_, C73G, AGG76TTC, \hat{ }TCCCA82; T65A, A83G; C70A, G74A; |
| | G7C, TC14AT, G17A, T34C; G7T, A33C, A36C, A76G; T-.3.CA, GC.7.A-, |
| | AG29GC, CA32TG; C2_A80G; -T.3.AC, C6T, C8_, G28C, G30C, CA32GT; |
| | G7C, A83G; C2_, C67A; T3G, A29C, T34G, G77A; C2G, A21G, T65C; |
| | G40A, T59A; \hat{ }A66, \hat{ }G66; G81A; C2_, A29G; \hat{ }T64, G81A; |
| | \hat{ }CGC2, CGC6_, GAG28_, \hat{ }AGG33; \hat{ }C77; T69A, A76G; \hat{ }T78, \hat{ }T78; C66A, \hat{ }C79; |
| | C2_, G7A, T34A; T3C, C6T, G30A, A33G, \hat{ }C55; GC7CG, GA28AG; |
| | T3C, G5C, GC7TA, G28T, C31G, A33G; \hat{ }T68, \hat{ }C77; \hat{ }T77, G77A; A27G, \hat{ }GT77; |
| | \hat{ }G66, \hat{ }T79, A88C; T9C, G69, \hat{ }A76; C68T, G75C; \hat{ }T81, \hat{ }T81; \hat{ }C66; T9C, G28C; |
| | T14A, A29C, C66T; \hat{ }A65; T3A, G5C, C8A, A29C, C31G, A33T; CT2_, T71A; |
| | G7C, C15G, A33T; G77A, \hat{ }T78; G63T, C82A; G7A, C15G, G54A, A60C, G79T; |
| | \hat{ }A13, \hat{ }G13; T72G, C73T; A36C, G54T; T3G, G7T; \hat{ }G65, T65C; T65G, C66G; |
| | G77C; T45G; C15A; C41T, G51A; T14A; C2T, G54T; A76T; T71A, A76G; |
| | \hat{ }G66, \hat{ }T79; \hat{ }A7, A29C; TGG3AAC, C8G, GA28CC, CCA31GTG; \hat{ }A1; \hat{ }T29; |
| | T71G, G74T; T45A; \hat{ }AT78, A88C; \hat{ }A3, GG4CC, C8_, GA28CG, CCA31GAT; |
| | C66T, C70G; C2_\hat{ }A66, \hat{ }C79; \hat{ }TA76, A88C; |
| | TG3GA, CG6GA, AG29TC, CA32TC; \hat{ }C80, A80G; G79C; C67G, \hat{ }G77; |
| | \hat{ }C66, G79A; G7A, T16C, \hat{ }T68; G7C, T9C, G17C, G75C; C2_\hat{ }T58; \hat{ }A65, \hat{ }C80; |
| | A1G, -C.68.GA; G17C, T65G; TG3CC, C6T, C8G, GAG28ACA, CA32GG; |
| | T72C, G75A; C64G, A80T; G7A, C66T; C66G, \hat{ }C79; C15A, G17A; \hat{ }AG66, A88C; |
| | A36_; G79T; T9C, G17C, \hat{ }T58; T10G, A29T; \hat{ }G69, \hat{ }C76; \hat{ }A69, A76_; |
| | G7A, A29G; A53_; T65G, \hat{ }A80; C70A, C73A; T59C, G74T; C67A; G54T, \hat{ }G56; |
| | \hat{ }G66; C2_, A29C; C38_, G54_; T3_, C6T, \hat{ }C8, \hat{ }G28, G30A, A33_; |
| | -TG.3.ACC, C6A, C8_\hat{ }CTC28, A29G, CCA31_; T9C, T14A; C64_; T14G, G54A; |
| | T71C, C73G, A83C; T9C, G17C; A53G, G54T; C66A, A80G; \hat{ }G63, \hat{ }G81; \hat{ }G1; |
| | \hat{ }C78, \hat{ }C78 |
| 2.3 to 2.4 | G7A, T9C; \hat{ }T67, \hat{ }G67; C2_, C67T; A80_; \hat{ }G1, A13C; \hat{ }G66, G79C; T69A, A76T; |
| | T9C, T14C; A76G, G78C; T16G, G17T; T69C, G77A; T65_, A80G; |
| | G7C, T14C, T34G; C66T, C67T; A53G, -A.80.TC; C67T, G77C; C73A, G74T; |
| | A36G, C68G; T9C, G17C, \hat{ }C78; TGG3GCT, C8G, \hat{ }AC28, CA32_; \hat{ }T18; |
| | \hat{ }C29, \hat{ }T29; \hat{ }GGGCG63, C68T, TTCGGA71_\hat{ }CCGCC82; |
| | T9C, G17C, C66T, A80G; \hat{ }A67, \hat{ }G67; C2_, G79T; |
| | T3A, CGC6GAT, GAG28ATC, A33T; C2_, A21G, G79C; C2_, A21G; C64T, G77A; |
| | C8A, G79C; C67G, \hat{ }A78, A80C; T69C, \hat{ }A70; G74T, G75C; \hat{ }T76, A76G; |
| | A76T, A80G; \hat{ }C64; \hat{ }C29, C50T; |
| | \hat{ }AGCTTA65, \hat{ }ATTG68, T69A, T72C, G77T, GA--.79.AGCT; A29T, G30A; |
| | T65C, A80C; \hat{ }C76, \hat{ }T76; T9C; \hat{ }G67, G79_; C68T, G79T; |
| | \hat{ }CTCA3, GCGC5_\hat{ }CAT28, CCA31_; \hat{ }GA70, A88C; -T.3.AC, GC.7.A-, |
| | GAG28TGC, CA32GG; A21G; \hat{ }G69, T69C; G7A, \hat{ }C66, \hat{ }G74; \hat{ }T65, \hat{ }A79; |
| | T65G; G74A, A76C; G74T, G75A; \hat{ }G68, G77T; T9G, G79T; \hat{ }AG67, A88C; \hat{ }C81; |
| | \hat{ }A67, G78T; C37A, G57T; G54C, G79T; G75T, G77A; G40A, TAG52CCT; \hat{ }G15; |
| | C67A, C68A; A36T, \hat{ }C55; A36T, T59A, T65C; C67T, \hat{ }G68; T71C, A76C; |
| | G7C, A29G, T65A; \hat{ }A78; T69C, G75T; \hat{ }TC66, A88C; CT2_, T59A; |
| | T9C, G17C, T65G; C70G, G75T; C2_, C73T, G75A; |
| | TG3CC, C6G, C8T, G28A, G30C, CA32GG; C64G, C66T; T11C, A29C; |
| | T9C, \hat{ }G15, G17C, T65C; T69G, G74T; \hat{ }GA65, A88C; G7C, A61G; \hat{ }T65, \hat{ }A80; |
| | C68_\hat{ }C79; G7A, \hat{ }T29, G79T; A27T; A1_, T9G, T59C; T14G, -G.79.TT; |
| | T14C, T16A; C70A, G74T; T65A, G78A; \hat{ }T65, \hat{ }G77; T9C, G17C, \hat{ }G68, G77C; |
| | C66A, \hat{ }A79; G7T, T9C, G17C; \hat{ }G69, A76T; C2_, A21C; \hat{ }T29, A29T; \hat{ }G69, \hat{ }T69; |
| | C6T, T10C, T84G; T65C, C67T; C15T; G78C; G7T, A27G, C44T; \hat{ }C68, \hat{ }A68; |
| | A1G, T9C, G17C, A76G; A36T, T59A; T14A, T16A; \hat{ }C66, G79_; |
| | -T.3.AA, G7_, AG29GC, CA32TG; C8G, \hat{ }A70, \hat{ }T75; C66A; \hat{ }C64, A80_; T69C; |
| | T71G, A76T; CT68TC, G74A; G54C, C68T; T9C, G17C, G81T; C2_, A13G; |
| | T65A, \hat{ }C81; \hat{ }C66, \hat{ }A78; \hat{ }C70, \hat{ }A75; \hat{ }T68, G77T; A29T, C50T, A53G, G79T; |
| | C68T, A76T; T16C, A18T, A80C; \hat{ }TGGAAGAT63, |
| | C---.66.TCGG, C68A, GGAGGGAG74_\hat{ }A83, A86C; |
| | -T.3.AA, G7_, AG29GC, CA32TT; T9G, A29G; C68A; A27C, A29T; A36T, G54C; |
| | \hat{ }A4; \hat{ }A73 |
| 2.2 to 2.3 | \hat{ }C66, A76_; \hat{ }G65; \hat{ }T1, T59C; A36T; T3C, GCG5CGA, AGC29TCG, A33G; |
| | T9C, TG16GC, C68T, G79A; G7A, T14A, G17A, T34A; T65G, G79T; |
| | G7C, TC14CT, G17A, T34A; T3C, C67A; G77C, G78T; C2T, \hat{ }G56; C6T, A83G; |
| | G7T, C8A; C66G, G79_; TG3_, C--.8.TCG, \hat{ }C28, \hat{ }C30, CA32_; C67T, T69G; |
| | CT2_, T9C; G78T, G79A; C2_, T9C, C15A, G17C; T9C, G17C, \hat{ }TG67; |
| | G75T, A76C; \hat{ }C76; G79A, A80T; TT71GG, G74C; C70_, G75C; \hat{ }G66, G79T; |
| | T34A, A60G; A29T, C64T, C66A; \hat{ }CT29, A88C; \hat{ }G69; A53C, G79T; \hat{ }T80, A80G; |
| | \hat{ }G67, C67A; C67A, G78C; T9C, G17C, C70T, T72A; -T.3.AC, GC.7.A-, A29_, |
| | A-33.GT; C2G, \hat{ }T58; A27G, \hat{ }A70; A39G, G78C; -G.78.AA, A80C; C66G, C67A; |
| | \hat{ }G68, \hat{ }A68; T69C, T71A; G7T, G40C, AG53GA; T9C, G17C, G79T; C8A, C66T; |
| | G74T, A80C; G7C, T14G; \hat{ }C77, G77T; G58T, G79C; T14C; \hat{ }T65, \hat{ }A80, A88C; |
| | C68A, \hat{ }C77; GC--.63.ATTA, CCC66ATT, G--GG.77.AATAT, GC81AT; |
| | T11G, A29T; T14A, T16G; T71C, G75A; \hat{ }T67, \hat{ }C78; T65C, G81A; G79C, A80T; |
| | \hat{ }C66, \hat{ }G74; A53C, G54A; \hat{ }C66, \hat{ }C79, A88C; G79C, A80G; |
| | T9C, G17C, \hat{ }C66, \hat{ }G74, A88C; C2_, T16C; T69G; \hat{ }G68, \hat{ }A76, A88C; |
| | T71A, G74C; G74T; G7T, C37A; \hat{ }CA68, A88C; \hat{ }T12, \hat{ }G12; A29T, C64T, C70T; |
| | G7C, A29G; G7A, T14A; T69C, C70G; G79T, A80C; C2T, G54C; \hat{ }T58; |
| | G7T, G30A, G81T; A29C, A83G; C2_, T69C; T3C, G5C, G7C, A29G, C31G, A33G; |
| | T72G; C64A; T34G, T59C; A1G, A60C; T65A, G79A; A27T, \hat{ }C29; \hat{ }G67, \hat{ }G77; |
| | \hat{ }G68, C68A; C64G; C66T, G77A; \hat{ }C64, \hat{ }A80; C2_, C73T; A29G; \hat{ }T7; |
| | A1_, A46C, T59C; T9C, G17C, A76T; G78C, A80G; \hat{ }C66, A76C; \hat{ }T29, \hat{ }T29; |
| | A27T, CT68TC, G74A; G75C, A76C; \hat{ }TT81, A88C; \hat{ }G77, A80G; \hat{ }C5, G7T; |
| | \hat{ }C66, T69C; C15A, T16A; C73T; \hat{ }A65, \hat{ }A80; \hat{ }T65, G79_; G40A, T52C; |
| | G7T, A60T; TG3GA, GC7CA, A29G, CA32TC; \hat{ }TA70, A88C; \hat{ }C66, \hat{ }A66; \hat{ }G67; |
| | A36C, \hat{ }T55, C68T; T65_; G63_, C82_; C2A, A29G |
| 2.1 to 2.2 | A83G; G75_; C68_, G79_; C2_, A46C; \hat{ }C4; \hat{ }A69, \hat{ }A69; G42A, C50T; |
| | A53G, \hat{ }T55; A36G, \hat{ }C58; TG3AC, C8A, GA28TC, CA32GG, T59C, C66A; |
| | C2_, A46C, C66T; C64T, G81T; \hat{ }A68, G77T; \hat{ }T80, A80T; T25G, A29T; |
| | G4A, C32T, G54_; \hat{ }T68; A76C, G78A; T9C, T14C, G17C; CT2_A33C; |
| | \hat{ }CA65, A88C; A60C; \hat{ }A69, \hat{ }T69; T9C, G17C, -T.65.GC; A18C, A61G, A80C; |
| | CT15TG, A21C; T72G, A76T; G7C, A29C; \hat{ }G79, \hat{ }C79; T69G, \hat{ }T76; C70A, G74C; |
| | T9G, A29C; C2_, G54A; C15G, T72A, G74A; \hat{ }A75; |
| | T3_, C6T, \hat{ }C8, A29_, C32A, \hat{ }C34; \hat{ }C29, A80C; G74A, A76T; C68T, T69C; |
| | T3_, C64T; A80T; CT2_, T9A; \hat{ }C29, A36C; \hat{ }GA67, A88C; T9C, G17C, T59A; |
| | A60T, C64T; T65A, G79T; A29C, T65C; \hat{ }T7, A13C; C8A, C82T; A76G, \hat{ }C77; |
| | T3G, GC7CT, GA28AG, CA32AC; ---TT.71.AAGAA, G75_; G7T, C15G; |
| | \hat{ }C79, \hat{ }C79; TG3GA, CG6AC, A29G, CA32TC, C68T, T72C; T72C; G63C, C82T; |
| | \hat{ }TG56, G57T; T14C, A29T, A36T; \hat{ }T68, \hat{ }T68; T69G, T71G; \hat{ }G66, C66T; |
| | \hat{ }G68, G77A; G54C, G79A; G7T, C67G; C66G, G78A; A60C, A76G, A80G; G40A, |
| | -A.76.CC; C2T, C67A, \hat{ }T78; T9C, G17C, G77A, G79T; G77T, G78A; \hat{ }T78, \hat{ }C78; |
| | \hat{ }T68, G77C; \hat{ }A67, \hat{ }G77; C73T, G75A; A29T, C66A, G74T; C2G, A36G; |
| | T3G, G5A, GC7CA, A29G, C31T, A33C; T69A, T71C; \hat{ }CG2, G5_, C8_, |
| | -G.28.CGC, CA32_; \hat{ }GT79, A88C; C68A, G77T; C64T; G40A, G77C; |
| | C68G, C70G; C2T, G78A; T9C, G17C, \hat{ }C66, A76C; G7T, A29G, C82T; |
| | C2_, T65G, A80G; TGG3GCT, C8G, \hat{ }CC28, CC31_; A29G, T69C, A80G; |
| | T34A, A36_; T9C, G17C, A27G; C15T, T16C; G7T, T9C, G17C, G40A, TA52AT; |
| | A36G, T71A; C6T; \hat{ }G69, A76_; C66A, G79A; \hat{ }C68, \hat{ }T68; A21T, C67A; |
| | A21C, T72G, G77T; T71G, A76G; C2T, G54A; T71G, G77A; |
| | T9C, G17C, A29G, G81A; G7A, A36T, G54C, C68T; T3A, T59A; \hat{ }G70; \hat{ }T77; |
| | \hat{ }T68, \hat{ }C77, A88C; TC14GT, T72C; T9C, G17C, T72_; \hat{ }C73; G7C, T14C; |
| | A36T, \hat{ }T58; G54T; T59C; A29C, C50T, A60T; G54A, C70G, \hat{ }T75; \hat{ }C66, G77C; |
| | C15G, G17C; C64G, \hat{ }C81; T3A, G5C, GC7AG, GA28CT, C31G, A33G; |
| | A29C, C32A; \hat{ }G28; A21G, A53G; G75A, A76T; G7C, TC14CT, G17C, T34A; |
| | G28A |

*mutated sequences are ‘;’-separated and multiple mutations per sequence are ‘,’-separated

TABLE 29
Guide 175 mutations and resulting relative enrichment

| Log₂ | |
|---|---|
| enrich- | |
| ment | Mutations on scaffold 175* (SEQ ID NO: 2239) |
| 3.2 to | C73A, \hat{ }T78; C6T, A29C, G71C, \hat{ }G80 |
| 3.5 | |
| 3.1 to | C17G, A87C; T3G, CGC6ACT, GAG28AGT, |
| 3.2 | A33C; G7T, C9T, C17G, CG81GA; |
| | T16G, A29C; C9T, C17G, C65A, A87G |
| 3.0 to | A68T, T83G; A27G, T92C; TGG3ATC, |
| 3.1 | GC7AG, GA28CT, CCA31GAT; |
| | \hat{ }C65, A87G; G7T, A29T; T3G, GC7AA, |
| | GA28TT, A33C; C9T, C17G, C65_; |
| | G7T, T14G; \hat{ }G54, G78T; C9T, |
| | C17G, \hat{ }A80; TC16AT, G64C |
| 2.9 to | C15T, T34A; C9T, C17G, A88T; G7A, |
| 3.0 | C15G; \hat{ }C76, \hat{ }G76; CT2_, C15_, T58A; |
| | C2_, C15G; C9T, A29C; C9T, C17G, A85T, |
| | A88T; C9T, C17G, \hat{ }CA63; G7T, C9G; |
| | A87T, A88C; C73G, G78A; A29T, |
| | A91G; TG3GA, G7A, A29G, CA32TC; |
| | \hat{ }G14, A29T, A87G; C9T, C17G, T74C; C2_, \hat{ }A53 |
| 2.8 to | C9T, A33T; G7T, T67G, G82C; \hat{ }T5, C9_, |
| 2.9 | GAGC28CGCA; G7T, \hat{ }A68, \hat{ }A82; |
| | G7T, \hat{ }C60; T14G, A29C; A29T, T66A; |
| | T3A, CG6TC, AG29GA, A33T; |
| | C2T, TC75AT; \hat{ }CG76, A88C; G7T, T14A, T83_; |
| | -T.3.GA, C6T, C9_, G28C, G30C, CA32TC; CT2_, |
| | C15T; TG3_, \hat{ }GT8, G30C, C32G; |
| | T14_, A29C; C9G, C17G, A29C, T79G; |
| | TG3AC, G7C, A29G, CA32GT, G86C, A88C; |
| | T3A, GC7CA, A29G, A33T; G7C, C80A |
| 2.7 to | G7T, A91C; \hat{ }C2, G4C, G7_, A29_, C32G, |
| 2.8 | \hat{ }G34; CT2_A88C; C65G, A88C; G7T, |
| | -T.79.AA; A29C; T3A, GC7CA, A29G; |
| | C8G, A29C, A88_; A29T; C2_, A29C; |
| | A29C, C31T, A33G; T14G, C15T; C9T, |
| | C15A; \hat{ }GA1, G7A, C15A, C17G; |
| | C15A, T16A; CT2_A29C; C9T, C17G, G78_; |
| | C9T, C17G, G-.78.AT; C73T, C76G |
| 2.6 to | C9T, C17G, C65_ \hat{ }A84; C9T, C17G, G70T, C81A; T74A, |
| 2.7 | T79A; T3C, C6T, AG29CA, A33G; G7A, \hat{ }T29; |
| | C76G, G77C; GG77CA, A87G; T16G, A29T; T3A, G5A, |
| | A29C, C31T, A33G; C9T, C17G, \hat{ }AA53; |
| | TG3CA, GC7AA, GA28TT, CA32TG; G7A, |
| | A29C; T3G, G7T; CT2_, A68G; |
| | T14_, A29T; C2_, C9T, C17G; \hat{ }G3, GC.7.-T, |
| | G28_ \hat{ }C34; G7T, \hat{ }T92; G7T, \hat{ }G69, G82T; |
| | \hat{ }GGCAGATCTGA64, T66C, A68C, GA71AG, |
| | \hat{ }C75, G77T, T79C, CGTAAGAA81_; |
| | T3A, C6G, AG29CC, A33T; C80T, \hat{ }A81; |
| | C81T; CT2_, C17A; C15A, T16G; |
| | C2_T16G; G71_, C80T; TG3AC, |
| | GC7AG, GA28CT, CA32GG; T3A, G5C, G7T, C31G, A33T; |
| | T3G, G7T, C9T, C17G; G64T, A85T; G7C, T14_; C9T, |
| | A29T; G7T, \hat{ }G14; A88G, \hat{ }C89; CT2_A33T; C81T, \hat{ }A82; |
| | C9T, C17G, A29C, C32A; C9T, C17G, \hat{ }GA77 |
| 2.5 to | G7C, C15G; C9T, C17G, TC75GT; TG3CA, |
| 2.6 | CG6GA, AG29TC, CA32GG; G7T; |
| | T14A, T16G; G7T, C9T, G71_, \hat{ }T79; C15A; |
| | CT2_, A33T, C73_; C2A, C9T, C17G; |
| | CGC6TCA, GAG28TGA; C15G, A29C; C2_, |
| | T16G, A91C; \hat{ }T81, C81T; |
| | TG3AA, A29C, CA32TG; G4A, G7T, C32T; |
| | T3C, CGC6GCT, GAG28AGC, A33G; |
| | T3A, G7A, A29T, A33G; -G.4.CC, G7_, |
| | AGCC29GCGG; C65T, G86_; C9T, \hat{ }A16; |
| | A36G, \hat{ }C57; A1_, T16G; C6T, G7T; \hat{ }G14, |
| | A29T; \hat{ }AT16, A88C; C8G, A29C; |
| | \hat{ }G64, A87C; \hat{ }G70, \hat{ }T79; T16A, \hat{ }C29; TG3GA, |
| | C6G, C8T, GAG28ACC, CA32TC, G71T; G7T, A29C; |
| | T3G, GCGSAGT, GC30CT, A33C; \hat{ }C2, \hat{ }T14, |
| | A29T; C9T, C17G, A88_; C9T, T16A |
| 2.4 to | TGG3ACA, A29C, CCA31TGT; T3_, G5A, |
| 2.5 | G7C, \hat{ }G9, \hat{ }C28, A29G, C31T, A33_; |
| | C15A, A29T; G64A, \hat{ }T65; CT2_, A27G; |
| | \hat{ }A16, \hat{ }T16; G7T, C15A; G7T, C9T, C17G; C2G, |
| | A29T, T66A; TG3GA, CGC6TTA, G28T, G30A, CA32TC; |
| | A1C, G82C; A27C, A29C; C9T, C17G, \hat{ }GA71; |
| | T3C, \hat{ }T6, CC.8.T-, C17G, GAG28AGA, A33G, \hat{ }G54; |
| | \hat{ }T16, A27T, A29C; G64C, \hat{ }A87; \hat{ }C14, A29C; |
| | \hat{ }A65, \hat{ }T65; C2T, C9T, C17G; C9T, C17A; |
| | G70A, C81A; C2G, A36T; G5C, C8G, GA28CC, |
| | CC31GA; C6T, A29C; C80T, \hat{ }G81; |
| | T-.3.CA, G7_, AG29GC, CA32TG; \hat{ }C78, |
| | G78A; G7A, T14_, CT65TC; -T.3.AA, G7_, |
| | AG29GC, CA32TG; \hat{ }C29, A29T; G7A, A29T; |
| | TG3GA, GC7CA, A29G, CA32TC; \hat{ }T64, G64A; |
| | C15A, A29C; T75A, G77T; \hat{ }A3, \hat{ }T3; A27T, A29C; |
| | T14A, A29C; T74C, G77A; G7C, A29G; C9T, C17_; |
| | G5A, G7A, A29T, C31T; \hat{ }C63, \hat{ }A63; G7T, A91G |
| 2.3 to | CT2_, G64T, T66G; G28T, A29C; T3G, G5T, |
| 2.4 | GC7CG, GA28CG, C31A; TG3AC, G7C, A29G, CA32GT; |
| | C9T, C15A, C17G, A29C, \hat{ }TG55, G57A; |
| | \hat{ }C14, A29T; C9T, C17G, GC64TG; G7A, \hat{ }T29, |
| | A36C; \hat{ }T16, \hat{ }G54; TG3CA, C8A, GA28TC, CA32GG; |
| | G7T, C9T, C69G; C9T, C17G, \hat{ }A70; |
| | A72_, T79G; T3A, G5T, |
| | C8T, GA28AC, C31A, A33T; C9T, C17G, A29C; \hat{ }G54; |
| | G7A, TC14CT, C17A; C9T, C17G; \hat{ }G70, \hat{ }T79, A88C; |
| | \hat{ }A64, \hat{ }G64; T14G, A29T; C9T, T16_; \hat{ }A14, \hat{ }T14; |
| | \hat{ }AC1, GCG.5.--T, GC30_, \hat{ }GT34; A29C, A91G; |
| | C2_, T14A; C9T, \hat{ }A17; C9T, C17G, G78A; |
| | T3G, G5A, A29C, C31T, A33C; C9T, \hat{ }G17; G7T, |
| | A29G; TG3GA, C6G, C8T, GAG28ACC, CA32TC; |
| | \hat{ }T1, CG6TC, C9T, C17G; C17A; \hat{ }T17, \hat{ }A17; |
| | T3A, G5C, GC7AG, GA28CT, C31G, A33G; \hat{ }GC72, |
| | A88C; T3G, G7T, A33C; TG3CA, CG6GA, AG29TC, |
| | CA32TG; T3G, G5C, C8G, GA28CC, C31G, A33C; |
| | \hat{ }T3, C80G; C9T, C17G, T45G, \hat{ }G54; |
| | C9T, C17G, A72C, T74G; |
| | G5C, C8G, GA28AC, C31G; A29T, G56T; G7T, C63A |
| 2.2 to | A36T, A85C, A87T; T14A, C17G; C9T, C17G, \hat{ }G54; |
| 2.3 | G4C, C8G, GA28AC, C32G, A87G; \hat{ }T72; A85C, |
| | A87C; G7T, T92C; C9T, C17G, \hat{ }C63; TG3AA, |
| | C6T, AG29CA, CA32TT; C9T, C17G, A85G, A88G; |
| | G64C, \hat{ }G88; G7A, \hat{ }T29, A68C; \hat{ }A13, T14C; C9T, |
| | C17G, G54, A85C, A88C; -GG.4.CAT, C9_, |
| | GAGCC28CGATG; TG3AC, C6A, AG29CT, CA32GG; |
| | C9T, \hat{ }C63; C9T, A88C; A27T, A29T; C9T, C17G, |
| | \hat{ }G54, A91C; G86A, A88T; TG3CA, GC7AA, GA28TT, |
| | CA32TG, C69T; T74G, G77T; TGG3ACA, C8G, |
| | GA28CC, CCA31TGG; G7A, C17A, \hat{ }G81; G7T, A59G; |
| | \hat{ }A65, \hat{ }G86; C73T, G78T; \hat{ }C72, \hat{ }T79; A1G, C9T, |
| | C17G; \hat{ }G1, C9T, C17G; \hat{ }G72, \hat{ }C72; C2_, A29T; |
| | \hat{ }T14, A29T; \hat{ }G64, \hat{ }T87; \hat{ }A65; \hat{ }C18, |
| | \hat{ }T18; \hat{ }G64, A88C; |
| | C9A, A29C, G57T; G7C, \hat{ }G28; G77A; G7A, TC14CT, |
| | C17G; C2_; G7C, T14A, \hat{ }T86; C9T, C17G, A53G; |
| | T3G, GC7CT, GA28AG, G86T; C9T, C17G, A29C, |
| | A91G; C9T, T16_, A91C; CT2_, \hat{ }G64, C65A; C15_; |
| | T16G, C17T; G7T, G28A |
| 2.1 to | C9T, C17G, A29T; A87C; \hat{ }CT18, A88C; C9T, C17G, |
| 2.2 | \hat{ }G64; C17G; C15T; \hat{ }T16, T79C; \hat{ }A64, G64A; |
| | A1C, T3G, C9T, C17G; GA28CC, \hat{ }T65; C15A, C17A; |
| | G78C, T79G; A29C, T58G; C2_, G7A, -C.65.AA; |
| | CT2_, A29T; T3A, A33T; G4A, CGC6GTA, |
| | G28T, G30C, C32T, T67_; C9T, C17G, C65_, A91C; \hat{ }T65, |
| | A87G; A88_; G7T, C9A; C9T, C17G, C65A; |
| | TG3GC, C6T, AG29CA, C32G; G7T, T16A; |
| | G7T, G70C, C80A; G7T, T14A; |
| | TG3AA, GC7CG, GA28CG, CA32TG; \hat{ }G54, A91C; |
| | C73_, G78_; T3C, GC5TG, C8T, GA26_, G30A, \hat{ }CG34; |
| | \hat{ }CT3, A29C; C2T, T14G; G7C, A29T; C9T, TC16GG; |
| | T3G, C8T, GA28AC, A33C; \hat{ }G16, \hat{ }T16; |
| | C9T, C17G, A36C; TGG3AAC, C8G, GAG28_, A---.33.GGGT; |
| | C9T, C17G, A87G; \hat{ }T72, T79G; \hat{ }G17, C17T; |
| | CT2_, A39C, A88C; T3G, A33C; T3_A33G; |
| | C-.2.TG, TC75CA; G7C, C9T, C17G, \hat{ }G92; C9T, C17G, |
| | G82C; C9A, A29C; C2_, C9T, C17G, A91C; |
| | C2_, A29C, A91C; CT2_, C9T, C17G; G7T, A60G; |
| | \hat{ }C71, \hat{ }T71; C2_, G77T, A91C; C2_, A29G; \hat{ }T71, |
| | C80G; T3A, G7A, A29G, A33T; C9T, A29G |
| 2.0 to | C65T, \hat{ }A66; CT2_, C15_, T58A, A72C; C9T, C17G, |
| 2.1 | C73A, C76A; C2_, A91C; C80T; T3A, |
| | G7C, C9T, C17G; \hat{ }C63, \hat{ }G88; G7T, A61T; |
| | GC62_, C65G, T67G, A72T, T79A, AAGA.84.---C, G89C; |
| | T3G, C9T; T16A, C17A; C6T, A29T; T3C, GC5CG, |
| | C8T, GAGC28ACCG, A33G; G7A, C15T; \hat{ }T2; C15G; |
| | C9G, A29T; C15T, A29T; G7T, \hat{ }C14; \hat{ }A64, A88T; |
| | A29C, G30A; C2_, A29C, A46C; C9T, C17G, A72T, |
| | G78A; \hat{ }A87, \hat{ }T87; C9T, A59C; TG3AC, C8A, |
| | GA28TC, CA32GG; C9T, C17G, \hat{ }G64, \hat{ }G88; |
| | A29C, G71A, C80T; T3C, A29T, AC68TA; \hat{ }A17; |
| | C9T, C17G, G64T, T66C; G7A, T16G; |
| | C17T, C65G, G86C; C69T, G82C; A1T, C2A; T14A, |
| | \hat{ }C29; \hat{ }A15, C15T; G7T, T16G; T3A, GC7CA, |
| | GA28TG, A33G; \hat{ }T81; T16C, A29C; A29C, A91C; |
| | G71A, A88T; \hat{ }C65, A87G, A91C; C9T, |
| | C17G, A29T, \hat{ }A53; G71T; \hat{ }A80, \hat{ }A80; |
| | C9T, C17G, A36G; C9T, C17G, T--.54.CTG; |
| | T16A, A29T; \hat{ }G77, T79C; |
| | C9T, C17G, G64C; TG3AC, CG6GA, |
| | AG29TC, CA32GG; A36T, C37T; |
| | A29C, \hat{ }C65, A85_; C15G, |
| | A29T; \hat{ }A70, C81T; A29T, A33G; C73A, C80T; C9T, |
| | C17G, G82_; C9T; C69T, A84G; C2_, C9T, C17G, A46C |
| 1.9 to | C2_, A29G, A91C; A68G, T83C; C9T, |
| 2.0 | T14A, C17A, \hat{ }AG85; \hat{ }T66, \hat{ }G85; G62T, CT65_, C69A, |
| | G71A, C80T, G82T, A85C, AGC88_; T3_, G5T, \hat{ }A8, |
| | -A.29.TC, C31A, A33_; G7A, T14C, C17A; |
| | T3G, CG6TC, AG29GA; \hat{ }T54; \hat{ }C8, \hat{ }T8; |
| | G7T, AA87TG; A72C, C73A; C2_, C6T; \hat{ }C29; |
| | G71C, C81_; C9T, C17G, G64_, A88_; C2_, A88T; |
| | T3G, G5C, GC7TG, G28C, C31G; |
| | C9T, C15T, C17G, A36C; G7T, T34G; |
| | T14A; \hat{ }T73, \hat{ }C78; \hat{ }G64; \hat{ }G15, C15T; |
| | A36C, \hat{ }A57; A-.72.GC, \hat{ }T79; |
| | T16A, A29C, \hat{ }A58; C9T, C17G, \hat{ }T52; C2_, A85T; |
| | \hat{ }C29, A29G; G7T, T14C; C2A, |
| | \hat{ }T57; G7T, C15G, T34G; T14G, C17T; T14C, C15T; |
| | T3G, G5A, GC7TA, G28T, C31T, |
| | A33C; \hat{ }C71, \hat{ }T79; \hat{ }T14, A29C; \hat{ }A1, A36C; |
| | \hat{ }C63, \hat{ }G89; G7C, A91G; T14C, A29C; |
| | C9T, C17G, G78T, C80T; \hat{ }G69, G82C; |
| | TGG3GCA, G7T, CCA31TGC; |
| | C6T, A29C, G71C, \hat{ }G80, A91C; A13C, A29C; |
| | \hat{ }C63, A88T; G7T, T14_; C2_, GG77AA; |
| | C9T, C17G, T58A; C2_, G77T; C2_, T3_; |
| | C9T, C17G, \hat{ }AA53, A88C; |
| | G7T, C9T; G7A; CG6GC, AG29GC, C32A; |
| | C63T, TTA66GCC, GA71_, TC79_, |
| | TAA83GGC, A87C, G89_; G7C, C17G; |
| | C2_, A46C; C9G, A29T, C37T, \hat{ }A56 |
| 1.8 to | \hat{ }G69, A72C, G82C; \hat{ }G70, T79G; |
| 1.9 | G7A, C15A; \hat{ }T36, \hat{ }A57; \hat{ } G70, \hat{ }C79; |
| | TGGCG3CACAT, GCCA30TGTG; |
| | G71A; TG3AC, C8A, A29C, CA32GT; |
| | T10G, A29C; \hat{ }A65, G77A, \hat{ }G86; |
| | C9T, C17G, A88_, A91C; \hat{ }C78, \hat{ }A78; |
| | G7T, C90T; T3G, G5A, GC7TG, |
| | G28C, C31T, A33C; G7T, C9G, G86T; |
| | A29C, C31T, A33C; A29C, |
| | G70A; A-.88.GC, A91C; \hat{ }A17, A36C; |
| | T3C, GCG5TGA, AGC29TCA, A33G; |
| | T3C, CGC6GCT, GAG28AGA, A33G, A88C; |
| | C35G, \hat{ }C58; T74A, G78C; |
| | C9T, CA17GT; G7A, C17G; C9T, C17G, \hat{ }GT70; |
| | CTG2_, A29C; C2_, A68G; \hat{ }T64, \hat{ }T88; |
| | T3G, A33T; C2_, T16G, A29C; \hat{ }A1; |
| | A36T, \hat{ }G55; C9T, C17G, C63A; |
| | C9T, A18G; C2T, A36T; \hat{ }A81, \hat{ }A81; |
| | C9T, T14G, C17G; -A.72.CC, |
| | A91C; A29T, T79G; G7A, A29T, A59G; G7C, \hat{ }C78; |
| | \hat{ }AG64, A88C; CT2_, C9T, C17G, |
| | C69T; C2_, A46C, A91C; \hat{ }C89, A91C; |
| | \hat{ }C29, A68C; C2_, G64T; -C.15.GT, |
| | A27C; CT2_, T10G, A88C; T14C, A29T; |
| | C9T, C17G, C76T; A84G, A87C; |
| | G7C, C9T, T14A, C17G, T34A; G70T, C81A; |
| | T14G; \hat{ }T3, A29T; G7T, \hat{ }T29; |
| | A29T, C65A, T67G; G64C, A87G; |
| | C9T, T14A, C17G; \hat{ }T57, |
| | A87G; TGG3ATC, A29C, CCA31GAT |
| 1.7 to | C2_, G70A; C9T, C17G, \hat{ }GA77, A88C; |
| 1.8 | C9G, C17G, A29C; \hat{ }T70, \hat{ }T81; |
| | G7C, C9T, C17G; T3G, CGC6TTG, |
| | G28C, G30A, A33C; \hat{ }A16, A68T; |
| | C9T, C17G, T67C; G7T, \hat{ }C14, A33C; |
| | G7A, T14_; \hat{ }C14, \hat{ }T14; |
| | C9T, C17G, GG77TT; C2T, C80T; \hat{ }T64, A88_; \hat{ }G54, |
| | A68C; G7T, CT9AG; |
| | C9T, C17G, T79G; T79G, C80T; \hat{ }AT3, |
| | A88C; \hat{ }AG54, A88C; C2G, A33C; |
| | C2_, A88T, A91C; C9T, C17G, T58C; C2_, C73T; |
| | TGG3CCC, C8G, GA28CC, CCA31GGG; |
| | G7T, T10G; C9T, C17G, \hat{ }A80, A91C; |
| | \hat{ }T64; T14_, A29C, A91C; G7A, G28T, |
| | AAAGCGCTTA59_; G7T, G71_; |
| | \hat{ }A17, \hat{ }A17; T14_, A29T, A91C; C17G, A72G, |
| | T74C; \hat{ }T88; CT2_, A94C; |
| | A27G, A29C; A85T, A87G; C9T, C17G, \hat{ }AA79; |
| | C9T, T14A, C17G, T34A, \hat{ }G64, G86T; C9T, |
| | C17G, T45G; C2_, C9T, C17G, C65T; |
| | \hat{ }G3, G5C, C9_, GA28CG, C32A; |
| | T74G, G78T; TG3_, --C.8.GCT, G28_, \hat{ }G33; |
| | A39T, T54A; C2_A72G; C9T, C15T, C17G; |
| | TG3CA, CG6GA, AG29GC, CA32TG; |
| | G64C, A88G; C15A, C17G; C2_, C65A; |
| | \hat{ } G64, G86A; \hat{ }C29, A36C; G64T, T66A; |
| | TG3GT, A29C, C32A; \hat{ }A64; C81G; |
| | C9T, A72T, T79C; C9T, C17G, G77T |
| 1.6 to | A72G; \hat{ }C14, A29C, A36C; T3C, C9T, |
| 1.7 | C17G; G4C, C8G, GA28AC, C32G; |
| | C2_, G71C, \hat{ }G80; C76T; C9T, T14A; |
| | C2G, C9T, C17G; G70T, C81G; C17G, \hat{ }T54; |
| | A72C; C2_, C9G, C17G; TG3GC, C8T, GA28AC, C32G; |
| | TGG3GCT, C8G, \hat{ }CC28, CC31_; |
| | C9T, C17G, A39T, A-.53.GC; \hat{ }T16; T67C, A87C; |
| | \hat{ }G81, C81T; C76G, G78C; A1C, G56A; |
| | TG3CA, GC7AG, GA28CT, CA32GG; |
| | C9T, C17G, C65G, \hat{ }A87; G86A, A88C; G7T, |
| | C9T, C17G, \hat{ }A72, G78A; \hat{ }G70, C80A; |
| | \hat{ }A17, A68C; C2_, C80G; \hat{ }C71, \hat{ }T79, A88C; |
| | C9T, C17G, \hat{ }T57; \hat{ }T2, C9T, C17G; T45G; |
| | G64C; T14_; C65T, G86A; C69T; \hat{ }C65; G64T, C65A; |
| | T3G, GC7CT, GA28AG; \hat{ }A1, \hat{ }A53; T3A, |
| | G5C, GC7AT, GA28AT, C31G, A33T; |
| | C9T, C17G, \hat{ }CA72; C9T, C17G, C73A, T79A; C2_, |
| | A53G; TGG3GTC, C8G, GA28CC, CC31GA; |
| | \hat{ }C5, G7T, C9T, C17G; G71T, C80T; |
| | C15T, T16G; G7C, C9T, C17G, C76A, |
| | G78T; G64T, T66C; \hat{ }C65, A91C; C73T; |
| | A72C, G78T; \hat{ }C63; A68G, C81T; \hat{ }GT87, |
| | A88C; C9T, C17G, \hat{ }A78; |
| | T3A, GC5AG, C8T, GAGC28ACCT, A33T; |
| | \hat{ }A1, \hat{ }T54; A29C, G56A; C2_, C80T; |
| | \hat{ }TA17, A88C; A72G, C73T; A29C, C31T, T83C; G7T, |
| | A27T; T3C, G7T, G40A, \hat{ }T54; A88C; ; G64T, A87C; |
| | T3_, \hat{ }T9, G28_, \hat{ }G32; \hat{ }GT16, A88C; |
| | -T.3.AC, G7A, C9_, GAG28TGC, |
| | CA32GG, A84C, G86T; \hat{ }T65; C76A, G77T; |
| | \hat{ }G14, A29C; G64C, A88C; A72_, |
| | T79G, A91C; \hat{ }C29, A68C, A72C; |
| | TG3AT, GC7TT, G28A, CA32AT; |
| | C9T, C17G, T--.54.CTG, A88C; G7T, A59C; |
| | CC8GT, C17G; G7C, T14C, \hat{ }T86; \hat{ }CA3, |
| | GC5_, C8G, GA26_, G30C, \hat{ }TG34 |
| 1.5 to | T3A, \hat{ }A5, G7_, AGC29GCT, |
| 1.6 | A33G; C9T, C17G, \hat{ }C73, G78C; G71A, A72G; |
| | AG27TA, A88T; G7T, A91T; \hat{ }T57, |
| | A91C; \hat{ }T2, A68C; \hat{ }T2, A36C; G7T, T10C; |
| | \hat{ }A64, A88G; TG3CA, C6T, C8T, |
| | GAG28ACA, CA32TG; \hat{ }T54, A68C, A72C; |
| | G7T, A61G; GCGC5CAAG, |
| | GAGC28CTTG; C6T, CT9TC, C17G, A29C; |
| | \hat{ }CA63, A88C; C2_, C9T, C17G, A36C; \hat{ }G64, \hat{ }G86; |
| | \hat{ }CGGCAGAT65, T67G, \hat{ }GC69, |
| | G70T, A72G, \hat{ }GCTC75, G77T, T79C, CGTAA81_; |
| | C73T, \hat{ }G74; T14G, T16A; \hat{ }AT14, |
| | A88C; G64C, A88T; C2_, A39T, \hat{ }A55; |
| | C2_, C15T; \hat{ }G70, C81T; \hat{ }A81, |
| | C81T; \hat{ }T72, A72T; C2_, C69T; T75G, T79G; |
| | A88_, A91C; \hat{ }T7, G7T; G7A, |
| | A29T, \hat{ }A77; CC8AT, C17G; C2_, T52C; |
| | G7A, C9T, TC16CG; G70A; |
| | C9T, C17G, AA87TC; \hat{ }A53, A91C; |
| | T3A, G5C, GC7CT, GA28AG, |
| | C31G, A33T; \hat{ }G70, \hat{ }C79, A88C; \hat{ }T72, \hat{ }G77; |
| | C9T, C17G, C69T; T-.3.CA, |
| | G7A, C9_, AG29GC, CA32TG; TGG.3.-AA, |
| | G9, \hat{ }CGC28, A29T, CCA31_; GCGC.62.--AA, |
| | T67C, C69A, GA71AC, TC79GT, |
| | G82T, AAGA.84.---G, GC89TT; |
| | A85G, A87G; TG3_, C--.8.TCG, |
| | GAG28CGA, C32G; T66C, A85G; |
| | \hat{ }A16, G86T, A88T; TT74GG, G--.77.AAC; |
| | C2_, T79C; C9T, \hat{ }A13, C17G, \hat{ }G54; |
| | \hat{ }C63, G64T; C2_, T83C; \hat{ }C73, |
| | \hat{ }C73; -T.3.AA, G7_, A29_, A-.33.GT, G70A; |
| | \hat{ }T16, A91C; \hat{ }T64, \hat{ }G64; T79C; |
| | C9T, C17G, G77A; \hat{ }T64, \hat{ }T64; C2_, G71A; |
| | T14C, C17G; G7C, TC14CA, C17G; A85C, A88C; |
| | \hat{ }A3, GG4TC, C9_, GA28CG, |
| | CCA31AAT; --C.63.TTT, C65_, CGGA.69.T---, |
| | TCCG.79.--- A, G86C, G89A; C9T, |
| | C17G, \hat{ }C57; C15G, T16A; C9T, C17G, \hat{ }CA64; |
| | AG39TA, T52C, T54A; C2A, A87G |
| 1.4 to | -C.15.GT, A36C; A29C, T83C; G7T, |
| 1.5 | A27G; \hat{ }C29, \hat{ }C29; \hat{ }T80, \hat{ }C80; |
| | TGGC3ACAG, GA26_, --A.33.TGG; |
| | A72G, \hat{ }T73; C9T, C17G, T66A, A85G; |
| | \hat{ }C15, \hat{ }G15; TG3_, --C.8.GCT, |
| | GAG28CGC, C32G; \hat{ }T19; G28A, A29C; |
| | \hat{ }G70, \hat{ }G80; CT2_, A36C, |
| | A39C; C9T, C17G, \hat{ }CC79; \hat{ }G54, A68C, A72C; |
| | \hat{ }CT78, A88C; T74G, G78C; |
| | TTC74AGG, \hat{ }AT78; C9T, C17G, C76G; |
| | \hat{ }GGCAGCTCTGA64, T66C, |
| | A68C, GA71AG, \hat{ }C75, G77T, T79C, |
| | CGTAAGAA81_; \hat{ }A1, A68C; \hat{ }A4; A72G, |
| | G78C; T3G, C8T, GA28CC, A33C; G7C, -C.80.AT; |
| | C9T, C17G, A59T; G26C, C93G; |
| | G7C, T14A, \hat{ }T86, A91C; \hat{ } G64, \hat{ }T87, A88C; |
| | A1G, A29C; C9T, C17G, \hat{ }AT78; |
| | G28T, GCCA30TTTG; C2_, T75A, G78A; |
| | TG3GA, CG6AC, AG29GT, |
| | CA32TC; A36G, \hat{ }C57, A91C; \hat{ }C72, A72C; |
| | C9T, C17G, \hat{ }G82; A27T; TG3CC, |
| | CGC6TTG, G28C, G30A, CA32GG, C80G; |
| | \hat{ }A1, \hat{ }A53, A88C; A72C, C80A; |
| | G7T, C73G; \hat{ }A15, A87G; T14_, \hat{ }C29; |
| | G7A, T14_, A91C; C15T, T16A; |
| | C15T, C17G; C65_, A88_A94C; \hat{ }A16; |
| | C9T, C17G, \hat{ }G54, A68C; -T.3.AC, |
| | G5A, C9_, GAG28CGT, CA32GG; \hat{ }T15, \hat{ }C15; |
| | C9T, T14A, C17G, T34C, \hat{ }G64, |
| | G86T; \hat{ }T71, C80G, A91C; -C.15.GT, A68C; |
| | \hat{ }G87, \hat{ }T87; C73_, G78_, A94C; |
| | C2G; G77C, T79A; G70C; A68G; \hat{ }T81, A91C; |
| | C9T, C17G, T79A; \hat{ }T72, \hat{ }T72 |
| 1.3 to | T66A, A88C; C76G, G77T; A53G, |
| 1.4 | A59C; CTG2_, G7T; A72_, \hat{ }T79; |
| | \hat{ }AA80, A88C; TGG3CAA, C8G, |
| | GA28CC, CCA31TGG; \hat{ }C78, \hat{ }T78; --G.28.TGA, |
| | T79C; \hat{ }T72, \hat{ }G77, A88C; A72G, \hat{ }C79; |
| | T3G, G5A, G7A, A29G, C31T, A33C; |
| | T14G, A21G; \hat{ }T2, A72C; G7T, T14G, \hat{ }CG64; |
| | T3G, G71A; G64A, A87G; T3C, C6T, AG29CC, A33G; |
| | T45A; G7A, C9T, T14A, C17G; TG3CT, |
| | CGC6TAT, GAG28ATA, CA32AG; |
| | C9T, C17G, \hat{ }T83; G7T, C9T, |
| | A53T; C9T, C17G, T75G; G7C, T14C, A72_; |
| | \hat{ }A65, A87G, \hat{ } 89; C9T, C17G, |
| | G70C, C81G; G7T, A59T; AG29CA, A72T, \hat{ }G77; |
| | T74C, G78A; C2A; C9T, C17G, C73T, |
| | T75G; \hat{ }G54, A72C; \hat{ }AA81, A88C; |
| | \hat{ }T54, A68C; C65A, G86A; \hat{ }A1, |
| | A72C; T3G, C9T, C17G; C2_, A33T; A87T; |
| | \hat{ }A65, \hat{ }T86; A53G; A85G, A87C; |
| | T3G, G5C, GC7TG, G28C, C31G, TC75_; |
| | -T.3.AC, G7A, C9_, GAG28TGC, |
| | CA32GG, G71T; G7C, C15A; G64A, A85G, A88_; |
| | \hat{ }A74; \hat{ }TG64, A88C; A29C, A60T; |
| | C9T, C17G, C80G; \hat{ }T64, \hat{ }A87; G7T, \hat{ }A59; |
| | G77C, G78C; A72C, \hat{ }T79; \hat{ }T73, \hat{ }C78, |
| | A88C; \hat{ }C29, A91C; \hat{ }A64, A88C; |
| | \hat{ }G54, T58A; TGGCG3CACTT, |
| | GCCA30AGTG; C9T, C17G, A21T; |
| | G4C, C8G, GA28AC, C32G, \hat{ }G82; |
| | A36C, A53G; C9T, C17G, G71T; |
| | C9T, CA17GT, T45A, G70C; \hat{ }A81; |
| | G7A, A72T; CT2_, T10G; G64T, A87G; |
| | \hat{ }G70, T79A; C2_, C9T, C17G, T52C; |
| | C2_, T45C; C9T, C17G, \hat{ }C35, A36G; |
| | G7T, T58A; \hat{ }A73, \hat{ }C73 |
| 1.2 to | C2G, C73G; G7T, \hat{ }T14; T75C, |
| 1.3 | C76T; \hat{ }A80, \hat{ }C80; A1_, A46C; C9T, C17_, A91C; |
| | C35G, \hat{ }C58, A68C; C2T, T3A; \hat{ }C29, |
| | A72C; T79G, C80A; G71A, C81_; G7T, G28T; |
| | CT2_, T45G; A29C, G92; C9T, C17G, T67C, A84G; |
| | T3C, \hat{ }T6, C9_, GAG28AGA, A33G; A36T; A85C, |
| | A88T; TG3GC, C6A, C8T, GAG28ACT, |
| | CA32GC; T10C, A29C; A1_, C2_; \hat{ }C65, A87T; |
| | A72T, C81T; C15A, T79A; \hat{ }GA1, |
| | G7A, C15A, C17G, A88C; \hat{ }A16, T16A; |
| | A29T, A60C; C76A, G78A; A29T, |
| | C31T; A29C, G86C; \hat{ }G70, T79G, A91C; |
| | \hat{ }T54, A72C; \hat{ }GAAC73, T74A, |
| | GG.77.C-; T14_, A29C, A46C; |
| | C9T, C17G, \hat{ }A72, \hat{ }A78; T14C, |
| | C15A; \hat{ }A17, \hat{ }G17; C9T, C17G, CG76AC; |
| | T74C, T79C; G7A, TC14AA, |
| | C17A; \hat{ }T64, \hat{ }A64; \hat{ }T81, \hat{ }A81; C2A, A36T; |
| | C9T, C17G, G82T; T74A, G77A; \hat{ }A1, |
| | A33C, A36C; G7C, TC14CT, T34A; |
| | A36T, A53G; \hat{ }A65, \hat{ }A84; A1_; G7T, \hat{ }T60; |
| | T3A, G5C, G7T, C31G, A33T, |
| | T52G, C54; T75G, G77T; G5C, G7A, A29T, C31T; |
| | TGGC3CCAG, C8T, GA26_, |
| | G30A, --A.33.TGG; C9T, \hat{ }C17; C2_, T14A, A91C; |
| | G77A, G78T; \hat{ }G64, G86A, A91C; |
| | T16A, C17G; C9T, C17G, T34A; A87G; A39G, |
| | -T.54.GC; A39G, -T.54.GC, A91C; \hat{ }A5, |
| | C6T, C9_, G28C, GC30CT; A72C, G77A; |
| | C2_, A91C, A94C; C2_, G7C; |
| | A84G; C73A, G78T; \hat{ }T78, \hat{ }A78; |
| | TGG3GTC, C8G, GA28AC, |
| | CCA31GAC; G7A, G14; C76T, G77A; C2_, G7T; |
| | G7A, T14A; \hat{ }A17, A68C, A72C; |
| | TGG3CCA, GC7CG, GA28CG, CCA31TGG; |
| | T79G; \hat{ }A72, C78; C15G, A29T, |
| | G57C, A59T; T14A, G74; G7T, C65T, A87C; |
| | C9T, C17G, G70T |

*mutated sequences are
‘;’-separated and multiple mutations per sequence are ‘,’-separated

### Example 21: The CcdB Selection Assay Identifies CasX Protein Variants with Improved dsDNA Cleavage or Improved Spacer Specificity at TTC, ATC, and CTC PAM Sequences

Experiments were conduected to identify the set of variants derived from CasX 515 (SEQ ID NO: 145) that are biochemically competent and that exhibit improved activity or improved spacer specificity compared to CasX 515 for double-stranded DNA (dsDNA) cleavage at target DNA sequences associated with a PAM sequence of either TTC or ATC or CTC. In order to accomplish this, first, a set of spacers was identified with survival above background levels in a CcdB selection experiment using CasX 515 and guide scaffold 174. Second, CcdB selections were performed with these spacers to determine the set of variants derived from CasX 515 that are biochemically competent for dsDNA cleavage at the canonical “wild-type” PAM sequence TTC. Third, CcdB selection experiments were performed to determine the set of variants of CasX 515 that enable improved dsDNA cleavage at either PAM sequences of type ATC or of type CTC. Fourth, plasmid counter-selection experiments were performed to determine the set of variants derived from CasX 515 that resulted in improved spacer specificity.

### Materials and Methods:

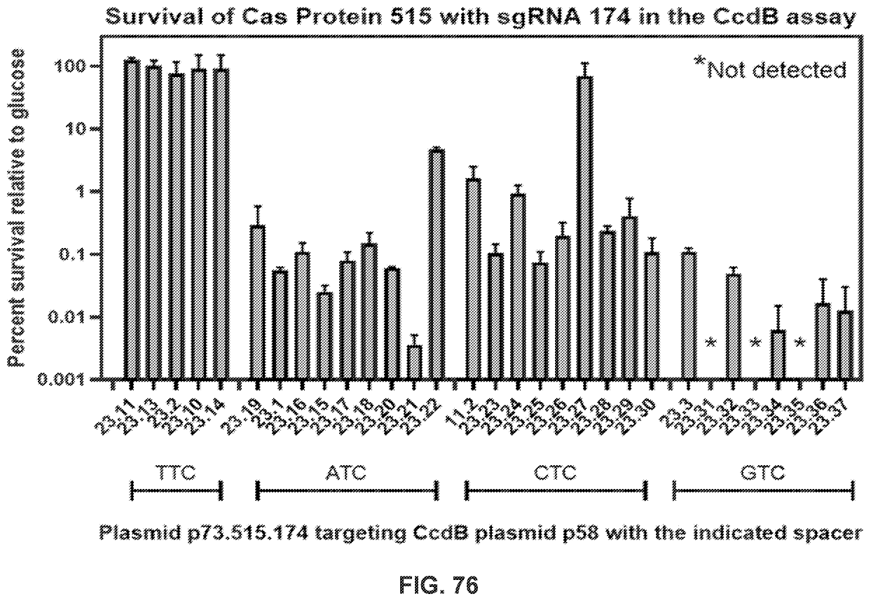

For CcdB selection experiments, 300 ng of plasmid DNA (p73) expressing the indicated CasX protein (or library) and sgRNA was electroporated into E. coli strain BW25113 harboring a plasmid expressing the CcdB toxic protein. After transformation, the culture was allowed to recover in glucose-rich media for 20 minutes at 37° C. with shaking, after which IPTG was added to a final concentration of 1 mM and the culture was further incubated for an additional 40 minutes. A recovered culture was then titered on LB agar plates (Teknova Cat #L9315) containing an antibiotic selective for the plasmid. Cells were titered on plates containing either glucose (CcdB toxin is not expressed) or arabinose (CcdB toxin is expressed), and the relative survival was calculated and plotted, as shown in FIG. 76. Next, a culture was electroporated and recovered as above, and a fraction of the recovery was saved for titering. The remainder of the recovered culture was split after the recovery period, and grown in media containing either glucose or arabinose, in order to collect samples of the pooled library either with no selection, or with strong selection, respectively. These cultures were harvested and the surviving plasmid pool was extracted using a Plasmid Miniprep Kit (QIAGEN) according to the manufacturer's instructions. The entire process was repeated for a total of three rounds of selection.

The final plasmid pool was isolated and a PCR amplification of the p73 plasmid was performed using primers specific for unique molecular identifier (UMI). These UMI sequences had been designed such that each specific UMI is associated with one and only one single mutation of the CasX 515 protein. Typical PCR conditions were used for the amplication The pool of variants of the CasX 515 contained many possible amino acid substitutions, as well as possible insertions, and single amino acid deletions in an approach termed Deep Mutational Evolution (DME). Amplified DNA product was purified with Ampure XP DNA cleanup kit, with elution in 30 μl of water. Amplicons were then prepared for sequencing with a second PCR to add adapter sequences compatible with next-generation sequencing (NGS) on either a MiSeq instrument or a NextSeq instrument (Illumina) according to the manufacturer's instructions. NGS of the prepared samples was performed. Returned raw data files were processed as follows: (1) the sequences were trimmed for quality and for adapter sequences; (2) the sequences from read 1 and read 2 were merged into a single insert sequence; and (3) each sequence was quantified for containing a UMI associated with a mutation relative to the reference sequence for CasX 515. Incidences of individual mutations relative to CasX 515 were counted. Mutation counts post-selection were divided by mutation counts pre-selection, and a pseudocount of ten was used to generate an “enrichment score”. The log base two (log₂) of this score was calculated and plotted as heat maps in which the enrichment score for biological replicates for a single spacer was determined at each amino acid position for insertions, deletions, or substitutions (not shown). The library was passed through the CcdB selection with two TTC PAM spacers performed in triplicate (spacers 23.2 AGAGCGTGATATTACCCTGT, SEQ ID NO: 41837, and 23.13 CCCTTTGACGTTGGAGTCCA, SEQ ID NO: 41838) and one TTC PAM spacer performed in duplicate (spacer 23.11 TCCCCGATATGCACCACCGG, SEQ ID NO: 41839), and the mean of triplicate measurements was plotted on a log₂ enrichment scale as a heatmap for the measured variants of CasX 515. Variants of CasX 515 that retained full cleavage competence compared to CasX 515 exhibited log 2 enrichment values around zero; variants with loss of cleavage function exhibited log 2 values less than zero, while variants with improved cleavage using this selection resulted in log 2 values greater than zero compared to the values of CasX 515. Experiments to generate additional heat maps (not shown) were performed using the following single spacers (11.2 AAGTGGCTGCGTACCACACC, SEQ ID NO: 41840; 23.27 GTACATCCACAAACAGACGA, SEQ ID NO: 41840; and 23.19 CCGATATGCACCACCGGGTA, SEQ ID NO: 41842, respectively) for selectivity.

For plasmid counter-selection experiments, additional rounds of bacterial selection were performed on the final plasmid pool that resulted from CcdB selection with TTC PAM spacers. The overall scheme of the counter-selection is to allow replication of only those cells of E. coli which contain two populations of plasmids simultaneously. The first plasmid (p73) expresses a CasX protein (under inducible expression by ATc) and a sgRNA (constitutively expressed), as well as an antibiotic resistance gene (chloramphenicol). Note that this plasmid can also be used for standard forward selection assays, such as CcdB, and that the spacer sequence is completely free to vary as desired by the experimentalist. The second plasmid (p74) serves only to express an antibiotic resistance gene (kanamycin) but has been modified to contain (or not contain) target sites matching the spacer encoded in p73. Furthermore, these target sites can be designed to incorporate “mismatches” relative to the spacer sequence, consisting of non-canonical Watson-Crick base-pairing between the RNA of the spacer and the DNA of the target site. If the RNP expressed from p73 is able to cleave a target site in p74, the cell will remain only resistant to chloramphenicol. In contrast, if the RNP cannot cleave the target site, the cell will remain resistant to both chloramphenicol and kanamycin. Finally, the dual plasmid replication system described above can be achieved in two ways. In sequential methods, either plasmid can be delivered to a cell first, after which the strain is made electrocompetent and the second plasmid is delivered (both by electroporation). Previous work has shown that either order of plasmid delivery is sufficient for successful counter-selection, and both schemes were performed: in an experiment named “Screen 5”, p73 is electroporated into competent cells harboring p74, while in Screen 6 the inverse is true. Cultures were electroporated, recovered, titered, and grown under selective conditions as above for a single round, and plasmid recovery followed by amplification, NGS, and enrichment calculation were also performed as above.

Finally, additional CcdB selections were performed in a similar manner, but with guide scaffold 235 and with alternative promoters WGAN45, Ran2, and Ran4, all targeting the toxic CcdB plasmid with spacer 23.2. These promoters are expected to more weakly express the guide RNA compared to the above CcdB selections and are thus expected to reduce the total concentration of CasX RNP in a bacterial cell. This physiological effect should reduce the overall survival of bacterial cells in the selective assay, thus increasing the dynamic range of enrichment scores and correlating more precisely with RNP nuclease activity at the TTC PAM spacer 23.2. For each promoter, three rounds of selection were performed in triplicate as above, and each round of experimentation resulted in enrichment data as above. These experiments are hereafter referred to as Screen 7.

### Results:

The results of the library screen heat maps demonstrated that CasX 515 complexed with guide scaffold 174 was capable of cleaving the CcdB expression plasmid when targeted using spacers (listed below) that target DNA sequences associated with TTC PAM sequences. In contrast, spacers utilizing alternative PAM sequences exhibited far more variable survival. ATC PAM spacers (listed below) ranged in survival from a few percent to much less than 0.1%, while CTC PAM spacers (listed below) enabled survival in a range from >50% to less than 1%. Finally, GTC PAM spacers (listed below) only enabled survival at or below 0.1%. These benchmarking data support the experimental design of this selection pipeline and demonstrate the robust selective power of the CcdB bacterial assay. Specifically, CasX proteins unable to cleave double-stranded DNA are de-enriched by at least four orders of magnitude, while CasX proteins biochemically competent for cleavage will survive the assay.

Heatmaps were used to identify the set of variants of CasX 515 that were biochemically competent for dsDNA cleavage at target DNA sequences associated with a TTC PAM sequence, as well as those variants exhibiting improved for dsDNA cleavage at target DNA sequences associated with PAM sequences of CTC (spacers 11.2 and 23.27) and ATC (spacer (23.19).

These three datasets, either individually, or combined, represent underlying biochemical differences between variants and identify regions of interest for future engineering of improved CasX therapeutics for human genome editing. As evidence for this, internal controls were included uniformly as part of the naive library, such as the presence of a stop codon at each position throughout the protein. These stop codons were consistently observed to be lost throughout rounds of selection, consistent with the expectation that partially truncated CasX 515 should not enable dsDNA cleavage. Similarly, variants with a loss of activity reflected in the heatmap data were observed to have become depleted during the selection, and thus have a severe loss of fitness for double-stranded DNA cleavage in this assay. However, variants with an enrichment value of one or greater (and a corresponding log 2 enrichment value of zero or greater) are, at minimum, neutral with respect to biochemical cleavage. Importantly, if one or more of the mutations identified in this specific subset of variants exhibit desirable properties for a therapeutic molecule, these mutations establish a structure-function relationship shown to be compatible with biochemical function. More specifically, these mutations can affect properties such as CasX protein transcription, translation, folding, stability, ribonucleoprotein (RNP) formation, PAM recognition, double-stranded DNA unwinding, non-target strand cleavage, and target strand cleavage.

For those variants competent for cleavage at sequences associated with CTC and ATC PAM sequences, enriched variants in these datasets (enrichment >1, equivalent to log 2 enrichment for values of approximately 0) represent mutations that specifically improve cleavage of CTC or ATC PAM target sites. Mutations meeting these criteria can be further subcategorized in two general ways: either the mutation improves cleavage rates by improving the recognition of the PAM (Type 1) or the mutation improves the overall cleavage rate of the molecule regardless of the PAM sequence (Type 2).

As examples of the first type, substitution mutations at position 223 were found to be enriched by several hundred-fold in all samples tested. This location encodes a glycine in both wild-type reference CasX proteins CasX 1 and 2, which is measured to be 6.34 angstroms from the −4 nucleotide position of the DNA non-target strand in the published CryoEM structure of CasX 1 (PDB ID: 6NY2). These substitution mutations at position 223 are thus physically proximal to the altered nucleotide of the novel PAM, and likely interact directly with the DNA. Further supporting this conclusion, many of the enriched substitutions encoded amino acids which are capable of forming additional hydrogen bonds relative to the replaced amino acid (glycine). These findings demonstrate that improved recognition of novel PAM sequences can be achieved in the CasX protein by introducing mutations that interact with one or both of the DNA strands, especially when physically proximal to the PAM DNA sequence (within ten angstroms). Additional features of the heat maps for ATC and CTC spacers represented mutations enabling increased recognition of non-canonical PAM sequences, but their mechanism of action has not yet been investigated.

As examples for the second type of mutation, the results of the heat maps were used to identify mutations that improve the overall cleavage rate compared to CasX 515, but without necessarily specifically recognizing the PAM sequence of the DNA. For example, a variant of CasX 515 consisting of an insertion of arginine at position 27 was measured to have an enrichment value greater than one in the selection with spacer 11.2 (CTC PAM) and spacer 23.19 (ATC PAM). This variant had previously been identified by a comparable selection on a CTC PAM spacer, where this mutation was enriched by orders of magnitude (data not shown). The position of this amino acid mutation is physically proximal (9.29 angstroms) to the DNA target strand at position −1 in the above structural model. These insights suggest a mechanism where the mature R-loop formed by CasX RNP with double-stranded DNA is stabilized by the side chain of the arginine, perhaps by ionic interactions of the positively charged side chain with the negatively charged backbone of the DNA target strand. Such an interaction is beneficial to overall cleavage kinetics without altering the PAM specificity. These data support the conclusion that some enriched mutations represent variants that improve the overall cleavage activity of CasX 515 by physically interacting with either or both of the DNA strands when physically proximal to them (within ten angstroms).

The data support the conclusion that many of the mutations measured to improve cleavage at sequences associated with the CTC or ATC PAM sequences identified from the heat maps can be classified as either of the two types of mutations specified above. For mutations of type one, variants consisting of mutations to position 223 with a large enrichment score in at least one of the spacers tested at CTC PAMs are listed in Table 30, with the associated maximum enrichment score. For mutations of type two, a smaller list of mutations was chosen systematically from among the thousands of enriched variants. To identify those mutations highly likely to improve the overall cleavage activity compared to CasX 515, the following approach was taken. First, mutations were filtered for those which were most consistently enriched across CTC or ATM PAM spacers. A lower bound (LB) was defined for the enrichment score of each mutation for each spacer. LB was defined as the combined log₂ enrichment score across biological triplicates, minus the standard deviation of the log₂ enrichment scores for the individual replicates. Second, the subset of these mutations was taken in which LB>1 for at least two out of three independent experimental datasets (one ATC PAM selection and two CTC PAM selections). Third, this subset of mutations was further reduced by excluding those for which a negative log₂ enrichment was measured in any of the three TTC PAM selections. Finally, individual mutations were manually selected based on a combination of structural features and strong enrichment score in at least one experiment. The resulting 274 mutations meeting these criteria are listed in Table 31, along with the maximum observed log 2 enrichment score from the two CTC or one ATC PAM experiments represented in the resulting heat maps, as well as the domain in which the mutation is located.

In contrast to Class I mutations, there exists another category of mutations that improve the ability of the CasX RNP to discriminate between on-target and off-target sites in genomic DNA, as determined by the spacer sequence, termed Class II, which improve the spacer specificity of the nuclease activity of the CasX protein. Two additional experiments were performed to specifically identify Class II mutations, where these experiments consisted of plasmid counter-selections and resulted in enrichment scores representing the sensitivity of the generated variant, compared to CasX 515, to a single mismatch between the spacer sequence of the guide RNA and the intended target DNA. The resulting enrichment scores were ranked for all observed mutations across the experimental data, and the following analyses were performed to identify a subset of mutations likely to improve the spacer specificity of the CasX protein without substantially reducing the nuclease activity at the desired on-target site. First, mutations from Screen 5 were ranked by their average enrichment score across three technical replicates using Spacer 23.2. Those mutations which were physically proximal to the nucleotide mismatch, as inferred from published models of the CasX RNP bound to a target site (PDB ID: 6NY2), were removed in order to discard those Class II mutations that might only confer improvements to specificity at Spacer 23.2 only, rather than universally across spacers. Finally, these Class II mutations were discarded if their cleavage activity at on-target TTC PAM sites was negatively impacted by the mutation if their average log 2 enrichment from the three TTC PAM CcdB selections was less than zero. The resulting mutations meeting these criteria are listed in Table 32, along with the maximum observed log₂ enrichment score from Screen 5 and the domain in which the mutation is located. Additionally, Class II mutations were identified from the counter-selection experiment Screen 6. These mutations were similarly ranked by their mean enrichment scores, but different filtering steps were applied. In particular, mutations were identified from each of the following categories: those with the highest mean enrichment scores from either Spacer 23.2, Spacer 23.11, or Spacer 23.13; those with the highest combined mean enrichment scores from Spacer 23.2 and Spacer 23.11; those with the highest combined mean enrichment scores from Spacer 23.11 and Spacer 23.13; or those with the highest combined mean enrichment scores from Spacer 23.2 in Screen 5 and Spacer 23.2 in Screen 6. These resulting mutations are listed in Table 32, along with the maximum observed log 2 enrichment score from Screen 6 and the domain in which the mutation is located.

In addition to the Class I or Class II mutations, there exists another category of mutations that has been directly observed to improve the dsDNA editing activity at TTC PAM sequences. These mutations, termed Class III mutations, demonstrated improved nuclease activity by way of exhibiting enrichment scores above that of CasX 515 when targeting the CcdB plasmid using Spacer 23.2 in Screen 7. A computational filtering step was used to identify a subset of these enriched mutations which are of particular interest. Specifically, mutations were identified that had an average enrichment value across three replicates that was greater than zero for each of the three promoters tested. Finally, features of the enrichment scores across the amino acid sequence were used to identify additional mutations at enriched positions. Example features of interest included the following: insertions or deletions at the junction of protein domains in order to facilitate topological changes; substitutions of an amino acid for proline in order to kink the polypeptide backbone; substitutions of an amino acid for a positively charged amino acid in order to add ionic bonding between the protein and the negatively charged nucleic acid backbone of either the guide RNA or either strand of the target DNA; deletions of an amino acid where consecutive deletions are both highly enriched; substitutions to a position that contains many highly enriched substitutions; substitutions of an amino acid for a highly enriched amino acid at the extreme N-terminus of the protein. These resulting mutations are listed in Table 33, along with the maximum observed log 2 enrichment score from Screen 6 and the domain in which the mutation is located.

TABLE 30
Mutations to CasX 515 (SEQ ID NO: 145)
that improve cleavage activity at CTC
PAM sequences by physically interacting
with the PAM nucleotides of the DNA

| | | | Maximum | |
|---|---|---|---|---|
| | | | observed log₂ | |
| | | | enrichment in | |
| Position | Reference | Alternate | Ccdb selections | Domain |
| 223 | G | Y | 4.6 | helical I-II |
| 223 | G | N | 5.7 | helical I-II |
| 223 | G | H | 4.2 | helical I-II |
| 223 | G | S | 4.6 | helical I-II |
| 223 | G | T | 3.8 | helical I-II |
| 223 | G | A | 6.3 | helical I-II |
| 223 | G | V | 3.6 | helical I-II |

TABLE 31
Mutations to CasX 515 (SEQ ID NO: 145) systematically identified from all datasets
to improve cleavage activity at ATC and CTC PAM sequences

| | | | Maximum observed log₂ | |
|---|---|---|---|---|
| Position | Reference | Alternate | enrichment in CcdB selections | Domain |
| 3 | — | G | 3.0 | OBD-I |
| 3 | I | G | 3.5 | OBD-I |
| 3 | I | E | 4.5 | OBD-I |
| 4 | — | G | 2.5 | OBD-I |
| 4 | K | G | 2.5 | OBD-I |
| 4 | K | P | 3.1 | OBD-I |
| 4 | K | S | 3.3 | OBD-I |
| 4 | K | W | 2.8 | OBD-I |
| 5 | — | P | 3.5 | OBD-I |
| 5 | — | G | 3.1 | OBD-I |
| 5 | R | S | 3.7 | OBD-I |
| 5 | — | S | 2.2 | OBD-I |
| 5 | R | A | 3.2 | OBD-I |
| 5 | R | P | 3.6 | OBD-I |
| 5 | R | G | 3.2 | OBD-I |
| 5 | R | L | 2.7 | OBD-I |
| 6 | I | A | 3.3 | OBD-I |
| 6 | — | G | 3.7 | OBD-I |
| 7 | N | Q | 3.1 | OBD-I |
| 7 | N | L | 2.7 | OBD-I |
| 7 | N | S | 3.7 | OBD-I |
| 8 | K | G | 3.3 | OBD-I |
| 15 | K | F | 3.0 | OBD-I |
| 16 | D | W | 2.8 | OBD-I |
| 16 | — | F | 4.2 | OBD-I |
| 18 | — | F | 3.5 | OBD-I |
| 28 | M | H | 2.5 | OBD-I |
| 33 | V | T | 2.0 | OBD-I |
| 34 | R | P | 3.6 | OBD-I |
| 36 | M | Y | 2.4 | OBD-I |
| 41 | R | P | 2.2 | OBD-I |
| 47 | L | P | 2.2 | OBD-I |
| 52 | E | P | 3.2 | OBD-I |
| 55 | — | P | 2.7 | OBD-I |
| 55 | PQ | — | 3.0 | OBD-I |
| 56 | Q | S | 1.9 | OBD-I |
| 56 | — | D | 2.5 | OBD-I |
| 56 | — | T | 2.8 | OBD-I |
| 56 | Q | P | 3.9 | OBD-I |
| 58 | — | A | 2.2 | helical I-I |
| 63 | R | S | 3.0 | helical I-I |
| 63 | R | Q | 2.7 | helical I-I |
| 72 | D | E | 2.7 | helical I-I |
| 81 | L | V | 2.8 | helical I-I |
| 81 | L | T | 2.7 | helical I-I |
| 85 | W | G | 3.2 | helical I-I |
| 85 | W | F | 2.7 | helical I-I |
| 85 | W | E | 2.9 | helical I-I |
| 85 | W | D | 3.1 | helical I-I |
| 85 | W | A | 2.8 | helical I-I |
| 85 | W | Q | 3.0 | helical I-I |
| 85 | W | R | 3.7 | helical I-I |
| 88 | F | M | 2.4 | helical I-I |
| 89 | Q | D | 2.5 | helical I-I |
| 93 | V | L | 1.9 | helical I-I |
| 109 | Q | P | 1.8 | NTSB |
| 115 | E | S | 1.8 | NTSB |
| 120 | G | D | 2.4 | NTSB |
| 133 | G | T | 2.2 | NTSB |
| 141 | L | A | 2.2 | NTSB |
| 168 | L | K | 3.1 | NTSB |
| 170 | A | Y | 2.7 | NTSB |
| 170 | A | S | 1.7 | NTSB |
| 175 | E | A | 2.0 | NTSB |
| 175 | E | D | 2.8 | NTSB |
| 175 | E | P | 3.8 | NTSB |
| 223 | G | — | 1.4 | helical I-II |
| 223 | G | S | 8.8 | helical I-II |
| 223 | G | T | 3.7 | helical I-II |
| 242 | S | T | 1.9 | helical I-II |
| 247 | I | T | 1.8 | helical I-II |
| 254 | V | T | 2.5 | helical I-II |
| 265 | L | Y | 1.9 | helical I-II |
| 288 | K | G | 4.2 | helical I-II |
| 288 | K | S | 4.0 | helical I-II |
| 291 | V | L | 2.6 | helical I-II |
| 303 | M | T | 2.3 | helical I-II |
| 303 | M | W | 2.7 | helical I-II |
| 328 | G | N | 3.3 | helical I-II |
| 331 | S | Q | 2.7 | helical I-II |
| 334 | — | A | 2.3 | helical II |
| 334 | LV | — | 3.0 | helical II |
| 335 | V | E | 2.8 | helical II |
| 335 | V | Q | 2.7 | helical II |
| 335 | V | F | 2.5 | helical II |
| 335 | V | — | 3.2 | helical II |
| 336 | E | P | 2.9 | helical II |
| 336 | E | — | 3.1 | helical II |
| 336 | E | D | 2.7 | helical II |
| 336 | E | L | 2.4 | helical II |
| 336 | E | R | 2.7 | helical II |
| 337 | R | N | 2.5 | helical II |
| 338 | Q | V | 2.5 | helical II |
| 338 | — | Q | 3.0 | helical II |
| 339 | — | G | 2.6 | helical II |
| 341 | — | H | 2.9 | helical II |
| 341 | — | A | 2.0 | helical II |
| 342 | V | D | 2.7 | helical II |
| 342 | — | T | 2.3 | helical II |
| 342 | V | — | 3.0 | helical II |
| 342 | — | F | 2.5 | helical II |
| 343 | — | D | 3.3 | helical II |
| 343 | D | — | 2.0 | helical II |
| 344 | W | — | 3.1 | helical II |
| 344 | W | T | 2.8 | helical II |
| 344 | W | H | 2.8 | helical II |
| 344 | — | P | 3.0 | helical II |
| 344 | — | G | 2.6 | helical II |
| 345 | — | R | 3.2 | helical II |
| 345 | W | P | 3.1 | helical II |
| 345 | W | D | 2.3 | helical II |
| 345 | — | D | 2.9 | helical II |
| 345 | W | L | 2.3 | helical II |
| 346 | — | P | 2.4 | helical II |
| 346 | — | D | 2.9 | helical II |
| 347 | M | — | 2.6 | helical II |
| 348 | — | T | 3.3 | helical II |
| 350 | N | I | 2.3 | helical II |
| 351 | V | N | 2.8 | helical II |
| 351 | V | H | 3.1 | helical II |
| 352 | K | D | 2.2 | helical II |
| 354 | L | D | 3.1 | helical II |
| 355 | I | S | 2.6 | helical II |
| 357 | E | C | 2.1 | helical II |
| 357 | E | P | 2.8 | helical II |
| 358 | K | T | 2.8 | helical II |
| 359 | K | E | 2.7 | helical II |
| 363 | K | L | 3.3 | helical II |
| 363 | K | Y | 2.2 | helical II |
| 367 | Q | D | 2.8 | helical II |
| 367 | Q | P | 3.0 | helical II |
| 369 | — | S | 2.6 | helical II |
| 369 | LA | — | 2.4 | helical II |
| 373 | K | L | 2.2 | helical II |
| 374 | — | R | 2.0 | helical II |
| 397 | Y | T | 2.5 | helical II |
| 400 | G | M | 2.0 | helical II |
| 402 | L | V | 2.4 | helical II |
| 403 | L | C | 2.3 | helical II |
| 404 | L | D | 2.5 | helical II |
| 404 | L | N | 2.5 | helical II |
| 404 | L | W | 2.3 | helical II |
| 404 | L | Y | 2.1 | helical II |
| 407 | E | F | 2.6 | helical II |
| 407 | E | L | 2.2 | helical II |
| 407 | E | Y | 2.6 | helical II |
| 411 | G | P | 2.6 | helical II |
| 411 | — | E | 3.2 | helical II |
| 413 | — | T | 2.7 | helical II |
| 413 | — | R | 2.4 | helical II |
| 413 | — | W | 3.0 | helical II |
| 413 | — | Y | 3.7 | helical II |
| 414 | — | W | 2.6 | helical II |
| 414 | — | Y | 3.1 | helical II |
| 414 | W | G | 3.0 | helical II |
| 414 | W | R | 2.6 | helical II |
| 416 | K | D | 2.7 | helical II |
| 416 | K | H | 2.0 | helical II |
| 416 | K | P | 2.6 | helical II |
| 416 | K | T | 2.3 | helical II |
| 417 | V | L | 2.6 | helical II |
| 417 | V | A | 2.5 | helical II |
| 418 | Y | C | 2.7 | helical II |
| 419 | D | G | 3.2 | helical II |
| 419 | D | M | 2.4 | helical II |
| 419 | D | P | 2.4 | helical II |
| 425 | I | C | 2.2 | helical II |
| 427 | K | T | 2.4 | helical II |
| 428 | K | R | 2.5 | helical II |
| 430 | E | G | 1.9 | helical II |
| 432 | L | A | 1.9 | helical II |
| 434 | K | H | 2.2 | helical II |
| 436 | I | T | 2.4 | helical II |
| 436 | I | S | 3.0 | helical II |
| 436 | I | Q | 2.7 | helical II |
| 437 | K | D | 3.1 | helical II |
| 442 | R | D | 2.5 | helical II |
| 442 | R | — | 2.7 | helical II |
| 446 | D | E | 2.3 | helical II |
| 446 | — | D | 2.3 | helical II |
| 450 | K | P | 2.3 | helical II |
| 452 | A | R | 2.0 | helical II |
| 453 | L | T | 3.2 | helical II |
| 456 | W | L | 2.2 | helical II |
| 457 | L | C | 2.2 | helical II |
| 459 | A | L | 2.0 | helical II |
| 461 | A | T | 2.7 | helical II |
| 461 | A | K | 2.1 | helical II |
| 465 | I | E | 3.1 | helical II |
| 465 | — | C | 2.9 | helical II |
| 466 | — | S | 3.5 | helical II |
| 466 | — | G | 2.5 | helical II |
| 467 | — | R | 2.4 | helical II |
| 467 | G | P | 2.0 | helical II |
| 468 | L | K | 3.6 | helical II |
| 468 | L | D | 3.2 | helical II |
| 468 | L | S | 3.0 | helical II |
| 468 | L | H | 3.3 | helical II |
| 470 | E | — | 2.4 | helical II |
| 472 | D | R | 2.2 | helical II |
| 472 | — | D | 2.4 | helical II |
| 473 | — | P | 2.6 | helical II |
| 474 | — | D | 2.7 | helical II |
| 475 | EF | — | 2.8 | helical II |
| 475 | — | Q | 2.7 | helical II |
| 476 | F | K | 2.8 | helical II |
| 476 | F | — | 2.2 | helical II |
| 477 | — | G | 2.8 | helical II |
| 479 | C | D | 3.1 | helical II |
| 480 | — | V | 2.2 | helical II |
| 480 | E | D | 2.3 | helical II |
| 481 | — | H | 2.2 | helical II |
| 481 | L | R | 2.9 | helical II |
| 482 | K | R | 2.1 | helical II |
| 483 | L | H | 2.7 | helical II |
| 484 | Q | C | 2.1 | helical II |
| 485 | K | P | 3.0 | helical II |
| 490 | L | S | 2.8 | helical II |
| 498 | E | L | 2.1 | helical II |
| 499 | — | F | 1.6 | helical II |
| 511 | K | T | 6.8 | OBD-II |
| 524 | — | P | 2.4 | OBD-II |
| 553 | — | S | 2.4 | OBD-II |
| 558 | — | R | 1.9 | OBD-II |
| 570 | M | T | 2.7 | OBD-II |
| 582 | I | T | 1.9 | OBD-II |
| 592 | Q | I | 2.1 | OBD-II |
| 592 | Q | F | 2.8 | OBD-II |
| 592 | Q | V | 2.0 | OBD-II |
| 592 | Q | A | 2.9 | OBD-II |
| 641 | — | R | 2.3 | OBD-II |
| 643 | — | D | 2.7 | OBD-II |
| 644 | — | W | 2.5 | OBD-II |
| 645 | — | A | 2.4 | OBD-II |
| 650 | — | I | 2.5 | RuvC-I |
| 651 | — | S | 2.4 | RuvC-I |
| 652 | — | T | 2.4 | RuvC-I |
| 652 | — | N | 2.3 | RuvC-I |
| 653 | — | R | 2.3 | RuvC-I |
| 653 | — | K | 2.2 | RuvC-I |
| 654 | — | H | 2.2 | RuvC-I |
| 654 | — | S | 2.3 | RuvC-I |
| 658 | V | L | 1.9 | RuvC-I |
| 695 | G | W | 1.4 | RuvC-I |
| 695 | G | R | 3.5 | RuvC-I |
| 708 | K | S | 3.0 | RuvC-I |
| 708 | K | T | 2.9 | RuvC-I |
| 708 | K | E | 3.1 | RuvC-I |
| 711 | V | A | 1.6 | RuvC-I |
| 726 | K | E | 2.0 | RuvC-I |
| 729 | N | G | 2.8 | RuvC-I |
| 736 | R | H | 2.7 | RuvC-I |
| 736 | R | G | 2.4 | RuvC-I |
| 771 | M | S | 3.7 | RuvC-I |
| 771 | M | A | 3.3 | RuvC-I |
| 792 | L | F | 2.5 | RuvC-I |
| 868 | V | D | 1.9 | TSL |
| 877 | — | A | 2.0 | TSL |
| 886 | T | E | 1.8 | TSL |
| 886 | T | D | 2.5 | TSL |
| 886 | T | N | 1.6 | TSL |
| 888 | G | D | 2.5 | TSL |
| 890 | S | — | 3.0 | TSL |
| 891 | G | — | 2.7 | TSL |
| 892 | — | E | 2.0 | TSL |
| 892 | — | N | 2.9 | TSL |
| 895 | S | I | 1.7 | TSL |
| 908 | E | D | 1.7 | TSL |
| 932 | S | M | 2.5 | RuvC-II |
| 932 | S | V | 2.6 | RuvC-II |
| 944 | — | L | 1.4 | RuvC-II |
| 947 | — | G | 1.9 | RuvC-II |
| 949 | T | — | 1.9 | RuvC-II |
| 951 | G | I | 3.7 | RuvC-II |

TABLE 32
Mutations to CasX 515 (SEQ ID NO: 145) systematically identified from all datasets
to improve spacer specificity

| | | | Maximum observed log₂ | |
|---|---|---|---|---|
| Position | Reference | Alternate | enrichment in counter-selections | Domain |
| 6 | I | L | 2.25 | OBD-I |
| 48 | — | P | 2 | OBD-I |
| 87 | — | G | 3.96 | helical I-I |
| 90 | K | V | 4.84 | helical I-I |
| 155 | F | V | 2.13 | NTSB |
| 215 | — | T | 2.03 | helical I-II |
| 216 | — | C | 3.03 | helical I-II |
| 220 | Y | F | 2.1 | helical I-II |
| 264 | S | H | 3.16 | helical I-II |
| 329 | — | Q | 2.71 | helical I-II |
| 343 | D | S | 2.69 | helical II |
| 346 | DM | — | 2.96 | helical II |
| 349 | — | P | 2.06 | helical II |
| 357 | — | G | 2.11 | helical II |
| 375 | QE | — | 2.34 | helical II |
| 378 | L | N | 2.38 | helical II |
| 389 | K | Q | 2.29 | helical II |
| 417 | — | L | 2.75 | helical II |
| 441 | E | L | 2.36 | helical II |
| 458 | R | D | 2.2 | helical II |
| 459 | A | E | 2.65 | helical II |
| 476 | FC | — | 2.34 | helical II |
| 503 | IL | — | 2.15 | OBD-II |
| 537 | K | G | 2.85 | OBD-II |
| 621 | L | T | 2.45 | OBD-II |
| 624 | — | A | 3 | OBD-II |
| 783 | L | Y | 2.08 | RuvC-I |
| 783 | — | P | 2.6 | RuvC-I |
| 787 | L | — | 2.49 | RuvC-I |
| 787 | L | R | 3.58 | RuvC-I |
| 787 | L | D | 5.58 | RuvC-I |
| 788 | — | Q | 2.65 | RuvC-I |
| 789 | — | R | 2.5 | RuvC-I |
| 789 | — | N | 2.71 | RuvC-I |
| 790 | E | N | 2.45 | RuvC-I |
| 792 | — | P | 2.85 | RuvC-I |
| 793 | P | A | 2.93 | RuvC-I |
| 795 | K | Q | 2.45 | RuvC-I |
| 796 | T | V | 2.75 | RuvC-I |
| 798 | — | R | 4.07 | RuvC-I |
| 799 | — | H | 2.79 | RuvC-I |
| 801 | T | Q | 3.16 | RuvC-I |
| 801 | — | H | 3.34 | RuvC-I |
| 801 | — | R | 2.86 | RuvC-I |
| 802 | — | L | 2.88 | RuvC-I |
| 802 | L | — | 2.87 | RuvC-I |
| 802 | — | W | 3.08 | RuvC-I |
| 803 | — | A | 3.19 | RuvC-I |
| 803 | — | F | 3.14 | RuvC-I |
| 803 | A | S | 5.79 | RuvC-I |
| 804 | Q | K | 3.05 | RuvC-I |
| 805 | Y | — | 3.29 | RuvC-I |
| 806 | T | Y | 3.07 | RuvC-I |
| 806 | T | F | 2.49 | RuvC-I |
| 807 | — | I | 3.21 | RuvC-I |
| 807 | S | P | 2.61 | RuvC-I |
| 809 | T | P | 3.2 | RuvC-I |
| 809 | — | N | 3.1 | RuvC-I |
| 810 | C | K | 3.19 | RuvC-I |
| 810 | C | M | 3.08 | RuvC-I |
| 811 | — | M | 2.51 | TSL |
| 812 | N | — | 3.07 | TSL |
| 812 | — | V | 2.68 | TSL |
| 813 | C | S | 2.3 | TSL |
| 814 | — | G | 3.15 | TSL |
| 814 | — | W | 3.04 | TSL |
| 815 | F | P | 3.09 | TSL |
| 817 | — | W | 2.87 | TSL |
| 828 | K | G | 1.99 | TSL |
| 906 | V | C | 2.01 | TSL |

TABLE 33
Mutations to CasX 515 (SEQ ID NO: 145) systematically identified from all datasets
to improve cleavage activity at TTC PAM sequences

| | | | Maximum observed log₂ | |
|---|---|---|---|---|
| | | | enrichment in Ccdb | |
| Position | Reference | Alternate | selections | Domain |
| 4 | K | W | 3.51 | OBD-I |
| 5 | R | P | 4.01 | OBD-I |
| 27 | — | P | 4.69 | OBD-I |
| 28 | M | P | 3.69 | OBD-I |
| 56 | Q | P | 3.78 | OBD-I |
| 85 | W | A | 3.96 | helical I-I |
| 102 | — | G | 4.75 | NTSB |
| 104 | — | I | 4.43 | NTSB |
| 104 | — | L | 4.52 | NTSB |
| 130 | S | — | 4.02 | NTSB |
| 151 | Y | T | 3.46 | NTSB |
| 168 | L | D | 3.32 | NTSB |
| 168 | L | E | 4.08 | NTSB |
| 188 | K | Q | 4.96 | NTSB |
| 190 | G | Q | 4.1 | NTSB |
| 223 | G | — | 1.63 | helical I-II |
| 235 | G | L | 4.64 | helical I-II |
| 235 | G | H | 4.97 | helical I-II |
| 239 | S | H | 3.93 | helical I-II |
| 239 | S | T | 4.97 | helical I-II |
| 245 | Q | H | 5 | helical I-II |
| 288 | K | D | 5.08 | helical I-II |
| 288 | K | E | 4.79 | helical I-II |
| 303 | M | R | 3.71 | helical I-II |
| 303 | M | K | 3.29 | helical I-II |
| 307 | L | K | 3.55 | helical I-II |
| 328 | G | R | 3.91 | helical I-II |
| 328 | G | K | 4.58 | helical I-II |
| 334 | — | H | 5.65 | helical II |
| 335 | — | D | 5.5 | helical II |
| 335 | V | P | 5.1 | helical II |
| 345 | — | Q | 5.22 | helical II |
| 441 | — | K | 5.07 | helical II |
| 477 | C | R | 2.94 | helical II |
| 477 | C | K | 3.49 | helical II |
| 502 | S | — | 4.04 | OBD-II |
| 503 | I | R | 3.72 | OBD-II |
| 503 | I | K | Not detected | OBD-II |
| 504 | L | — | 4.24 | OBD-II |
| 542 | R | E | 4.54 | OBD-II |
| 563 | K | — | 3.25 | OBD-II |
| 593 | — | A | 1.83 | OBD-II |
| 610 | K | Q | 3.46 | OBD-II |
| 615 | R | Q | 3.67 | OBD-II |
| 643 | — | A | 2.42 | OBD-II |
| 697 | S | R | 2.67 | RuvC-I |
| 697 | S | K | 2.55 | RuvC-I |
| 906 | V | T | 4.65 | TSL |

1. A polynucleotide comprising the following component sequences:
a. a first AAV inverted terminal repeat (ITR) sequence;
b. a second AAV ITR sequence;
c. a first promoter sequence;
d. a sequence encoding a CRISPR protein;
e. a sequence encoding a first guide RNA (gRNA); and,
f. optionally, at least one accessory element sequence,
wherein the polynucleotide is configured for incorporation into a recombinant adeno-associated virus (AAV).

2. The polynucleotide of claim 1, wherein the sequences encoding the CRISPR protein and the first gRNA are less than about 3100, less than about 3090, less than about 3080, less than about 3070, less than about 3060, less than about 3050, or less than about 3040 nucleotides in combined length.

3. The polynucleotide of claim 1 or 2, wherein the sequences of the first promoter and the at least one accessory element have greater than at least about 1300, at least about 1350, at least about 1360, at least about 1370, at least about 1380, at least about 1390, at least about 1400, at least about 1500, at least about 1600 nucleotides, at least 1650, at least about 1700, at least about 1750, at least about 1800, at least about 1850, or at least about 1900 nucleotides in combined length.

4. The polynucleotide of claim 1 or 2, wherein the sequences of the first promoter and the at least one accessory element have greater than 1314 nucleotides in combined length.

5. The polynucleotide of claim 1 or 2, wherein the sequences of the first promoter and the at least one accessory element have greater than 1381 nucleotides in combined length.

6. The polynucleotide of any one of claims 1-5, wherein the first promoter sequence and the sequence encoding the CRISPR protein are operably linked.

7. The polynucleotide of claim 6, wherein the first promoter is a pol II promoter.

8. The polynucleotide of claim 6 or claim 7, wherein the first promoter is selected from the group consisting of polyubiquitin C (UBC) promoter, cytomegalovirus (CMV) promoter, simian virus 40 (SV40) promoter, chicken beta-Actin promoter and rabbit beta-Globin splice acceptor site fusion (CAG), chicken β-actin promoter with cytomegalovirus enhancer (CB7), PGK promoter, Jens Tornoe (JeT) promoter, GUSB promoter, CBA hybrid (CBh) promoter, elongation factor-1 alpha (EF-1alpha) promoter, beta-actin promoter, Rous sarcoma virus (RSV) promoter, silencing-prone spleen focus forming virus (SFFV) promoter, CMVd1 promoter, truncated human CMV (tCMVd2), minimal CMV promoter, hepB promoter, chicken β-actin promoter, HSV TK promoter, Mini-TK promoter, minimal IL-2 promoter, GRP94 promoter, Super Core Promoter 1, Super Core Promoter 2, Super Core Promoter 3, adenovirus major late (AdML) promoter, MLC promoter, MCK promoter, GRK1 protein promoter, Rho promoter, CAR protein promoter, hSyn Promoter, U1a promoter, Ribosomal Protein Large subunit 30 (Rpl30) promoter, Ribosomal Protein Small subunit 18 (Rps18) promoter, CMV53 promoter, minimal SV40 promoter, CMV53 promoter, SFCp promoter, Mecp2 promoter, pJB42CAT5 promoter, MLP promoter, EFS promoter, MeP426 promoter, MecP2 promoter, MHCK7 promoter, beta-glucuronidase (GUSB) promoter, CK7 promoter, and CK8e promoter.

9. The polynucleotide of claim 8, wherein the first promoter is a truncated variant of the UBC, CMV, SV40, CAG, CB7, PGK, JeT, GUSB, CB, EF-1alpha, beta-actin, RSV, SFFV, CMVd1, tCMVd2, minimal CMV, chicken β-actin, HSV TK, Mini-TK, minimal IL-2, GRP94, Super Core Promoter 1, Super Core Promoter 2, MLC, MCK, GRK1 protein Rho, CAR protein, hSyn, U1a, Ribosomal Protein Large subunit 30 (Rpl30), Ribosomal Protein Small subunit 18 (Rps18), CMV53, minimal SV40, CMV53, SFCp, pJB42CAT5, MLP, EFS, MeP426, MecP2, MHCK7, CK7, or CK8e promoter.

10. The polynucleotide of claim 7 or claim 8, wherein the first promoter sequence has less than about 400 nucleotides, less than about 350 nucleotides, less than about 300 nucleotides, less than about 200 nucleotides, less than about 150 nucleotides, less than about 100 nucleotides, less than about 80 nucleotides, or less than about 40 nucleotides.

11. The polynucleotide of claim 7 or claim 8, wherein the first promoter sequence has between about 40 to about 585 nucleotides, between about 100 to about 400 nucleotides, or between about 150 to about 300 nucleotides.

12. The polynucleotide of any one of claims 1-11, wherein the first promoter is selected from the group consisting of SEQ ID NOS: 40370-40400 as set forth in Table 8, or a sequence having at least 85%, at least 90%, at least 95%, at least 95%, at least 96%, at least 97%, at least 98%, or at least 99% identity thereto.

13. The polynucleotide of any one of claims 1-12, wherein the first promoter is selected from the group consisting of SEQ ID NOS: 41030-41044 as set forth in Table 24, or a sequence having at least 85%, at least 90%, at least 95%, at least 95%, at least 96%, at least 97%, at least 98%, or at least 99% identity thereto.

14. The polynucleotide of any one of claims 1-13, wherein the at least one accessory element is operably linked to the sequence encoding the CRISPR protein.

15. The polynucleotide of any one of claims 1-14, further comprising a second promoter.

16. The polynucleotide of claim 15, wherein the second promoter sequence and the sequence encoding the first gRNA are operably linked.

17. The polynucleotide of claim 15 or claim 16, wherein the second promoter is a pol III promoter.

18. The polynucleotide of any one of claims 15-17, wherein the second promoter is selected from the group consisting of U6, mini U61, mini U62, mini U63, BiH1 (Bidrectional H1 promoter), BiU6 (Bidirectional U6 promoter), gorilla U6, rhesus U6, human 7sk, and human H1 promoters.

19. The polynucleotide of claim 18, wherein the second promoter is a truncated variant of the U6, mini U61, mini U62, mini U63, BiH1, BiU6, gorilla U6, rhesus U6, human 7sk, or human H1 promoters.

20. The polynucleotide of claim 18 or claim 19, wherein the second promoter sequence has less than about 250 nucleotides, less than about 220 nucleotides, less than about 200 nucleotides, less than about 160 nucleotides, less than about 140 nucleotides, less than about 130 nucleotides, less than about 120 nucleotides, less than about 100 nucleotides, less than about 80 nucleotides, or less than about 70 nucleotides.

21. The polynucleotide of claim 18 or claim 19, wherein the second promoter sequence has between about 70 to about 245 nucleotides, between about 100 to about 220 nucleotides, or between about 120 to about 160 nucleotides.

22. The polynucleotide of any one of claims 15-21, wherein the second promoter sequence is selected from the group consisting SEQ ID NOS: 40401-40420 and 41010-41029 as set forth in Table 9, or a sequence having at least 85%, at least 90%, at least 95%, at least 95%, at least 96%, at least 97%, at least 98%, or at least 99% identity thereto.

23. The polynucleotide of any one of claims 15-22, wherein the second promoter enhances transcription of the first gRNA.

24. The polynucleotide of any one of claims 15-23, wherein the sequences of the first promoter and the second promoter are greater than at least about 1300, at least about 1350, at least about 1360, at least about 1370, at least about 1380, at least about 1390, at least about 1400, at least about 1500, at least about 1600 nucleotides, at least 1650, at least about 1700, at least about 1750, at least about 1800, at least about 1850, or at least about 1900 nucleotides in combined length.

25. The polynucleotide of any one of claims 15-24, wherein the sequences of the first promoter, the second promoter and the at least one accessory element are greater than at least about 1300, at least about 1350, at least about 1360, at least about 1370, at least about 1380, at least about 1390, at least about 1400, at least about 1500, at least about 1600 nucleotides, at least 1650, at least about 1700, at least about 1750, at least about 1800, at least about 1850, or at least about 1900 nucleotides in combined length.

26. The polynucleotide of any one of claims 15-25, wherein the sequences of the first promoter, the second promoter, and the at least one accessory element are greater than 1314 nucleotides in combined length.

27. The polynucleotide of any one of claims 15-26, wherein the sequences of the first promoter, the second promoter, and the at least one accessory element are greater than 1381 nucleotides in combined length.

28. The polynucleotide of any one of claims 1-27, comprising two or more accessory element sequences.

29. The polynucleotide of claim 28, wherein the sequences of the first promoter, the second promoter, and the two or more accessory elements are greater than at least about 1300, at least about 1350, at least about 1360, at least about 1370, at least about 1380, at least about 1390, at least about 1400, at least about 1500, at least about 1600, at least 1650, at least about 1700, at least about 1750, at least about 1800, at least about 1850, or greater than at least about 1900 nucleotides in combined length.

30. The polynucleotide of claim 28, wherein the sequences of the first promoter, the second promoter, and the two or more accessory elements are greater than 1314 nucleotides in combined length.

31. The polynucleotide of claim 28, wherein the sequences of the first promoter, the second promoter, and the two or more accessory elements are greater than 1381 nucleotides in combined length.

32. The polynucleotide of any one of claim 15-31, wherein at least 25%, 26%, 27%, 28%, 29%, 30%, 31%, 32%, 33%, 34%, or at least 35% or more of the length of the polynucleotide sequence comprises the sequences of the first and second promoters and the at least one accessory element.

33. The polynucleotide of any one of claims 1-32, wherein the accessory elements are selected from the group consisting of a poly(A) signal, a gene enhancer element, an intron, a posttranscriptional regulatory element (PTRE), a nuclear localization signal (NLS), a deaminase, a DNA glycosylase inhibitor, a stimulator of CRISPR-mediated homology-directed repair, and an activator of transcription, and a repressor of transcription.

34. The polynucleotide of any one of claims 1-32, wherein the accessory elements enhance the transcription, transcription termination, expression, binding of a target nucleic acid, editing of a target nucleic acid, or performance of the CRISPR protein as compared to an otherwise identical polynucleotide lacking said accessory elements.

35. The polynucleotide of claim 34, wherein the enhanced performance is an increase in editing of a target nucleic acid by the expressed CRISPR protein and the first gRNA in an in vitro assay of at least about 10%, at least about 20%, at least about 30%, at least about 40%, at least about 50%, at least about 60%, at least about 70%, at least about 80%, at least about 90%, at least about 100%, at least about 150%, at least about 200%, or at least about 300%.

36. The polynucleotide of any one of claims 1-35, wherein the encoded CRISPR protein is a Class 2 CRISPR protein.

37. The polynucleotide of claim 36, wherein the encoded CRISPR protein is a Class 2, Type V CRISPR protein.

38. The polynucleotide of claim 37, wherein the encoded Class 2, Type V CRISPR protein comprises:
a. a NTSB domain comprising a sequence of QPASKKIDQNKLKPEMDEKGNLTTAGFACSQCGQPLFVYKLEQVSEKGKAYTNYFGRC NVAEHEKLILLAQLKPEKDSDEAVTYSLGKFGQ (SEQ ID NO: 41818), or a sequence having at least 80% at least 90%, at least 95%, at least 96%, at least 97%, at least 98% or at least 99% identity thereto;
b. a helical I-II domain comprising a sequence of RALDFYSIHVTKESTHPVKPLAQIAGNRYASGPVGKALSDACMGTIASFLSKYQDIIIEH QKVVKGNQKRLESLRELAGKENLEYPSVTLPPQPHTKEGVDAYNEVIARVRMWVNLN LWQKLKLSRDDAKPLLRLKGFPSF (SEQ ID NO: 41819), or a sequence having at least 80%, at least 90%, at least 95%, at least 96%, at least 97%, at least 98% or at least 99% identity thereto;
c. a helical II domain comprising a sequence of PLVERQANEVDWWDMVCNVKKLINEKKEDGKVFWQNLAGYKRQEALRPYLSSEEDR KKGKKFARYQLGDLLLHLEKKHGEDWGKVYDEAWERIDKKVEGLSKHIKLEEERRSE DAQSKAALTDWLRAKASFVIEGLKEADKDEFCRCELKLQKWYGDLRGKPFAIEAE (SEQ ID NO: 41820), or a sequence having at least 80%, at least 90%, at least 95%, at least 96%, at least 97%, at least 98% or at least 99% identity thereto; and
d. a RuvC-I domain comprising a sequence of SSNIKPMNLIGVDRGENIPAVIALTDPEGCPLSRFKDSLGNPTHILRIGESYKEKQRTIQAK KEVEQRRAGGYSRKYASKAKNLADDMVRNTARDLLYYAVTQDAMLIFENLSRGFGRQ GKRTFMAERQYTRMEDWLTAKLAYEGLPSKTYLSKTLAQYTSKTC (SEQ ID NO: 41821), or a sequence having at least 80%, at least 90%, at least 95%, at least 96%, at least 97%, at least 98% or at least 99% identity thereto.

39. The polynucleotide of claim 38, wherein the encoded Class 2, Type V CRISPR protein comprises an OBD-I domain comprising a sequence of QEIKRINKIRRRLVKDSNTKKAGKTGPMKTLLVRVMTPDLRERLENLRKKPENIPQ (SEQ ID NO: 41822), or a sequence having at least 80%, at least 90%, at least 95%, at least 96%, at least 97%, at least 98%, or at least 99% identity thereto.

40. The polynucleotide of claim 38 or claim 39, wherein the encoded Class 2, Type V CRISPR protein comprises an OBD-II domain comprising a sequence of NSILDISGFSKQYNCAFIWQKDGVKKLNLYLIINYFKGGKLRFKKIKPEAFEANRFYT VINKKSGEIVPMEVNFNFDDPNLIILPLAFGKRQGREFIWNDLLSLETGSLKLANGRV IEKTLYNRRTRQDEPALFVALTFERREVLD (SEQ ID NO: 41823), or a sequence having at least 80%, at least 90%, at least 95%, at least 96%, at least 97%, at least 98%, or at least 99% identity thereto.

41. The polynucleotide of any one of claims 38-40, wherein the encoded Class 2, Type V CRISPR protein comprises a helical I-I domain comprising a sequence of PISNTSRANLNKLLTDYTEMKKAILHVYWEEFQKDPVGLMSRVA (SEQ ID NO: 41824), or a sequence having at least 80%, at least 90%, at least 95%, at least 96%, at least 97%, at least 98%, or at least 99% identity thereto.

42. The polynucleotide of any one of claims 38-41, wherein the encoded Class 2, Type V CRISPR protein comprises a TSL domain comprising a sequence of SNCGFTITSADYDRVLEKLKKTATGWMTTINGKELKVEGQITYYNRYKRQNVVKD LSVELDRLSEESVNNDISSWTKGRSGEALSLLKKRFSHRPVQEKFVCLNCGFETH (SEQ ID NO: 41825), or a sequence having at least 80%, at least 90%, at least 95%, at least 96%, at least 97%, at least 98%, or at least 99% identity thereto.

43. The polynucleotide of any one of claims 38-42, wherein the encoded Class 2, Type V CRISPR protein comprises a RuvC-II domain comprising a sequence of ADEQAALNIARSWLFLRSQEYKKYQTNKTTGNTDKRAFVETWQSFYRKKLKEVWK PAV (SEQ ID NO: 41826), or a sequence having at least 80%, at least 90%, at least 95%, at least 96%, at least 97%, at least 98%, or at least 99% identity thereto.

44. The polynucleotide of any one of claims 38-43, wherein the encoded Class 2, Type V CRISPR protein comprises the sequence of SEQ ID NO: 145, or a sequence having at least 80%, at least 90%, at least 95%, at least 96%, at least 97%, at least 98%, or at least 99% identity thereto.

45. The polynucleotide of any one of claims 38-44, wherein the encoded Class 2, Type V CRISPR protein comprises at least one modification in one or more domains.

46. The polynucleotide of claim 45, wherein the at least one modification comprises:
a. at least one amino acid substitution in a domain;
b. at least one amino acid deletion in a domain;
c. at least one amino acid insertion in a domain; or
d. any combination of (a)-(c).

47. The polynucleotide of claim 45 or claim 46, comprising a modification at one or more amino acid positions in the NTSB domain relative to SEQ ID NO: 41818 selected from the group consisting of P2, S4, Q9, E15, G20, G33, L41, Y51, F55, L68, A70, E75, K88, and G90.

48. The polynucleotide of claim 47, wherein the one or more modifications at one or more amino acid positions in the NTSB domain are selected from the group consisting of an insertion of G at position 2, an insertion of I at position 4, an insertion of L at position 4, Q9P, E15S, G20D, a deletion of S at position 30, G33T, L41A, Y51T, F55V, L68D, L68E, L68K, A70Y, A70S, E75A, E75D, E75P, K88Q, and G90Q relative to SEQ ID NO: 41818.

49. The polynucleotide of any one of claims 45-48, comprising a modification at one or more amino acid positions in the helical I-II domain relative to SEQ ID NO: 41819 selected from the group consisting of 124, A25, Y29 G32, G44, S48, S51, Q54, 156, V63, S73, L74, K97, V100, M112, L 116, G137, F138, and S140.

50. The polynucleotide of claim 49, wherein the one or more modifications at one or more amino acid positions in the helical I-II domain are selected from the group consisting of an insertion of T at position 24, an insertion of C at position 25, Y29F, G32Y, G32N, G32H, G32S, G32T, G32A, G32V, a deletion of G at position 32, G32S, G32T, G44L, G44H, S48H, S48T, S51T, Q54H, I56T, V63T, S73H, L74Y, K97G, K97S, K97D, K97E, V100L, M112T, M112W, M112R, M112K, L116K, G137R, G137K, G137N, an insertion of Q at position 138, and S140Q relative to SEQ ID NO: 41819.

51. The polynucleotide of any one of claims 45-50, comprising a modification at one or more amino acid positions in the helical II domain relative to SEQ ID NO: 41820 selected from the group consisting of L2, V3, E4, R5, Q6, A7, E9, V10, D11, W12, W13, D14, M15, V16, C17, N18, V19, K20, L22, I23, E25, K26, K31, Q35, L37, A38, K41, R42, Q43, E44, L46, K57, Y65, G68, L70, L71, L72, E75, G79, D81, W82, K84, V85, Y86, D87, 193, K95, K96, E98, L100, K102, 1104, K105, E109, R110, D114, K 118, A120, L121, W124, L125, R126, A127, A129, 1133, E134, G135, L136, E138, D140, K141, D142, E143, F144, C145, C147, E148, L149, K150, L151, Q152, K153, L158, E166, and A167.

52. The polynucleotide of claim 51, wherein the one or more modifications at one or more amino acid positions in the helical II domain are selected from the group consisting of an insertion of A at position 2, an insertion of H at position 2, a deletion of L at position 2 and a deletion of V at position 3, V3E, V3Q, V3F, a deletion of V at position 3, an insertion of D at position 3, V3P, E4P, a deletion of E at position 4, E4D, E4L, E4R, R5N, Q6V, an insertion of Q at position 6, an insertion of G at position 7, an insertion of H at position 9, an insertion of A at position 9, VD10, an insertion of T1 at position 0, a deletion of V at position 10, an insertion of F at position 10, an insertion of D at position 11, a deletion of D at position 11, D11S, a deletion of W at position 12, W12T, W12H, an insertion of P at position 12, an insertion of Q at position 13, an insertion of G at position 12, an insertion of R at position 13, W13P, W13D, an insertion of D at position 13, W13L, an insertion of P at position 14, an insertion of D at position 14, a deletion of D at position 14 and a deletion of M at position 15, a deletion of M at position 15, an insertion of T at position 16, an insertion of P at position 17, N18I, V19N, V19H, K20D, L22D, 123S, E25C, E25P, an insertion of G at position 25, K26T, K27E, K31L, K31Y, Q35D, Q35P, an insertion of S at position 37, a deletion of L at position 37 and a deletion of A at position 38, K41L, an insertion of R at position 42, a deletion of Q at position 43 and a deletion of E at position 44, L46N, K57Q, Y65T, G68M, L70V, L71C, L72D, L72N, L72W, L72Y, E75F, E75L, E75Y, G79P, an insertion of E at position 79, an insertion of T at position 81, an insertion of R at position 81, an insertion of W at position 81, an insertion of Y at position 81, an insertion of W at position 82, an insertion of Y at position 82, W82G, W82R, K84D, K84H, K84P, K84T, V85L, V85A, an insertion of L at position 85, Y86C, D87G, D87M, D87P, I93C, K95T, K96R, E98G, L100A, K102H, I104T, I104S, I104Q, K105D, an insertion of K at position 109, E109L, RI 10D, a deletion of R at position 110, D 14E, an insertion of D at position 114, K 118P, A120R, L121T, W124L, L125C, R126D, A127E, A127L, A129T, A129K, I133E, an insertion of C at position 133, an insertion of S at position 134, an insertion of G at position 134, an insertion of R at position 135, G135P, L136K, L136D, L136S, L136H, a deletion of E at position 138, D140R, an insertion of D at position 140, an insertion of P at position 141, an insertion of D at position 142, a deletion of E at position 143+a deletion of F at position 144, an insertion of Q at position 143, F144K, a deletion of F at position 144, a deletion of F at position 144 and a deletion of C at position 145, C145R, an insertion of G at position 145, C145K, C147D, an insertion of V at position 148, E148D, an insertion of H at position 149, L149R, K150R, L151H, Q152C, K153P, L158S, E166L, and an insertion of F at position 167 relative to SEQ ID NO: 41820.

53. The polynucleotide of any one of claims 45-52, comprising a modification at one or more amino acid positions in the RuvC-I domain relative to SEQ ID NO: 41821 selected from the group consisting of 14, K5, P6, M7, N8, L9, V12, G49, K63, K80, N83, R90, M125, and L146.

54. The polynucleotide of claim 53, wherein the one or more modifications at one or more amino acid positions in the RuvC-I domain are selected from the group consisting of an insertion of I at position 4, an insertion of S at position 5, an insertion of T at position 6, an insertion of N at position 6, an insertion of R at position 7, an insertion of K at position 7, an insertion of H at position 8, an insertion of S at position 8, V12L, G49W, G49R, S51R, S51K, K62S, K62T, K62E, V65A, K80E, N83G, R90H, R90G, M125S, M125A, L137Y, an insertion of P at position 137, a deletion of L at position 141, L141R, L141D, an insertion of Q at position 142, an insertion of R at position 143, an insertion of N at position 143, E144N, an insertion of P at position 146, L146F, P147A, K149Q, T150V, an insertion of R at position 152, an insertion of H153, T155Q, an insertion of H at position 155, an insertion of R at position 155, an insertion of L at position 156, a deletion of L at position 156, an insertion of W at position 156, an insertion of A at position 157, an insertion of F at position 157, A157S, Q158K, a deletion of Y at position 159, T160Y, T160F, an insertion of I at position 161, S161P, T163P, an insertion of N at position 163, C164K, and C164M relative to SEQ ID NO: 41821.

55. The polynucleotide of any one of claims 45-54, comprising a modification at one or more amino acid positions in the OBD-I domain relative to SEQ ID NO: 41822 selected from the group consisting of I3, K4, R5, 16, N7, K8, K15, D16, N18, P27, M28, V33, R34, M36, R41, L47, R48, E52, P55, and Q56.

56. The polynucleotide of claim 55, wherein the one or more modifications at one or more amino acid positions in the OBD-I domain are selected from the group consisting of an insertion of G at position 3, I3G, I3E, an insertion of G at position 4, K4G, K4P, K4S, K4W, K4W, R5P, an insertion of P at position 5, an insertion of G at position 5, R5S, an insertion of S at position 5, R5A, R5P, R5G, R5L, I6A, I6L, an insertion of G at position 6, N7Q, N7L, N7S, K8G, K15F, D16W, an insertion of F at position 16, an insertion of F18, an insertion of P at position 27, M28P, M28H, V33T, R34P, M36Y, R41P, L47P, an insertion of P at position 48, E52P, an insertion of P at position 55, a deletion of P at position 55 and a deletion of Q at position 56, Q56S, Q56P, an insertion of D at position 56, an insertion of T at position 56, and Q56P relative to SEQ ID NO: 41822.

57. The polynucleotide of any one of claims 45-56, comprising a modification at one or more amino acid positions in the OBD-II domain relative to SEQ ID NO: 41823 selected from the group consisting of S2, I3, L4, K11, V24, K37, R42, A53, T58, K63, M70, 182, Q92, G93, K110, L121, R124, R141, E143, V144, and L145.

58. The polynucleotide of claim 57, wherein the one or more modifications at one or more amino acid positions in the OBD-II domain are selected from the group consisting of a deletion of S at position 2, I3R, I3K, a deletion of I at position 3 and a deletion of L4, a deletion of L at position 4, K11T, an insertion of P at position 24, K37G, R42E, an insertion of S at position 53, an insertion of R at position 58, a deletion of K at position 63, M70T, I82T, Q92L, Q92F, Q92V, Q92A, an insertion of A at position 93, K110Q, R115Q, L121T, an insertion of A at position 124, an insertion of R at position 141, an insertion of D at position 143, an insertion of A at position 143, an insertion of W at position 144, and an insertion of A at position 145 relative to SEQ ID NO: 41823.

59. The polynucleotide of any one of claims 45-58, comprising a modification at one or more amino acid positions in the TSL domain relative to SEQ ID NO: 41825 selected from the group consisting of S1, N2, C3, G4, F5, 17, K18, V58, S67, T76, G78, S80, G81, E82, S85, V96, and E98.

60. The polynucleotide of claim 59, wherein the one or more modifications at one or more amino acid positions in the OBD-II domain are selected from the group consisting of an insertion of M at position 1, a deletion of N at position 2, an insertion of V at position 2, C3S, an insertion of G at position 4, an insertion of W at position 4, F5P, an insertion of W at position 7, K18G, V58D, an insertion of A at position 67, T76E, T76D, T76N, G78D, a deletion of S at position 80, a deletion of G at position 81, an insertion of E at position 82, an insertion of N at position 82, S85I, V96C, V96T, and E98D relative to SEQ ID NO: 41825.

61. The polynucleotide of any one of claims 45-60, wherein the expressed Class 2, Type V CRISPR protein exhibits an improved characteristic relative to SEQ ID NO: 2 or SEQ ID NO: 145, wherein the improved characteristic comprises increased binding affinity to a gRNA, increased binding affinity to the target nucleic acid, improved ability to utilize a greater spectrum of PAM sequences in the editing of the target nucleic acid, improved unwinding of the target nucleic acid, increased editing activity, improved editing efficiency, improved editing specificity for cleavage of the target nucleic acid, decreased off-target editing or cleavage of the target nucleic acid, increased percentage of a eukaryotic genome that can be edited, increased activity of the nuclease, increased target strand loading for double strand cleavage, decreased target strand loading for single strand nicking, increased binding of the non-target strand of DNA, improved protein stability, increased protein:gRNA (RNP) complex stability, and improved fusion characteristics.

62. The polynucleotide of claim 61, wherein the improved characteristic comprises increased cleavage activity at a target nucleic sequence comprising an TTC, ATC, GTC, or CTC PAM sequence.

63. The polynucleotide of claim 62, wherein the improved characteristic comprises increased cleavage activity at a target nucleic acid sequence comprising an ATC or CTC PAM sequence relative to cleavage activity of the sequence of SEQ ID NO: 145.

64. The polynucleotide of claim 63, wherein the improved cleavage activity is an enrichment score (log 2) of at least about 1.5, at least about 2.0, at least about 2.5, at least about 3, at least about 3.5, at least about 4, at least about 4.5, at least about 5, at least about 6, at least about 7, at least about 8 or more greater compared to score of the sequence of SEQ ID NO: 145 in an in vitro assay.

65. The polynucleotide of claim 63, wherein the improved characteristic comprises increased cleavage activity at a target nucleic acid sequence comprising an CTC PAM sequence relative to the sequence of SEQ ID NO: 145.

66. The polynucleotide of claim 65, wherein the improved cleavage activity is an enrichment score (log₂) of at least about 2, at least about 2.5, at least about 3, at least about 3.5, at least about 4, at least about 4.5, at least about 5, or at least about 6 or more greater compared to the score of the sequence of SEQ ID NO: 145 in an in vitro assay.

67. The polynucleotide of claim 62, wherein the improved characteristic comprises increased cleavage activity at a target nucleic acid sequence comprising an TTC PAM sequence relative to the sequence of SEQ ID NO: 145.

68. The polynucleotide of claim 67, wherein the improved cleavage activity is an enrichment score of at least about 1.5, at least about 2.0, at least about 2.5, at least about 3, at least about 3.5, at least about 4, at least about 4.5, at least about 5, or at least about 6 log₂ or more greater compared to the sequence of SEQ ID NO: 145 in an in vitro assay.

69. The polynucleotide of claim 61, wherein the improved characteristic comprises increased specificity for cleavage of the target nucleic acid sequence relative to the sequence of SEQ ID NO: 145.

70. The polynucleotide of claim 69, wherein the increased specificity is an enrichment score of at least about 2.0, at least about 2.5, at least about 3, at least about 3.5, at least about 4, at least about 4.5, at least about 5, or at least about 6 log 2 or more greater compared to the sequence of SEQ ID NO: 145 in an in vitro assay.

71. The polynucleotide of claim 61, wherein the improved characteristic comprises decreased off-target cleavage of the target nucleic acid sequence.

72. The polynucleotide of claim 37, wherein the encoded Class 2, Type V CRISPR protein is selected from the group consisting of Cas12f, Cas12j (CasPhi), and CasX.

73. The polynucleotide of claim 72, wherein the encoded CasX comprises a sequence selected from the group consisting of SEQ ID NOS: 1-3, 49-160, and 40208-40369, or a sequence having at least 85%, at least 90%, at least 95%, at least 95%, at least 96%, at least 97%, at least 98%, or at least 99% identity thereto.

74. The polynucleotide of claim 72, wherein the encoded CasX comprises a sequence selected from the group consisting of the sequences of SEQ ID NOS: 1-3, 49-160, 40208-40369 and 40828-40912.

75. The polynucleotide of claim 72, wherein the CasX sequence of the polynucleotide comprises a sequence selected from the group consisting of SEQ ID NOS: 40577-40588, as set forth in Table 21, or a sequence having at least 85%, at least 90%, at least 95%, at least 95%, at least 96%, at least 97%, at least 98%, or at least 99% identity thereto.

76. The polynucleotide of claim 72, wherein the CasX sequence of the polynucleotide comprises a sequence selected from the group consisting of SEQ ID NOS: 40577-40588, as set forth in Table 21.

77. The polynucleotide of any one of claims 1-76, wherein the polynucleotide encodes one or more NLS linked to the sequence encoding the CRISPR protein.

78. The polynucleotide of claim 77, wherein the sequences encoding the one or more NLS are positioned at or near the 5′ end of the sequence encoding the CRISPR protein.

79. The polynucleotide of claim 77, wherein the sequences encoding the one or more NLS are positioned at or near at the 3′ end of the sequence encoding the CRISPR protein.

80. The polynucleotide of claim 78 or claim 79, wherein the polynucleotide encodes at least two NLS, wherein the sequences encoding the at least two NLS are positioned at or near the 5′ and 3′ ends of the sequence encoding the CRISPR protein.

81. The polynucleotide of any one of claims 77-80, wherein the one or more encoded NLS are selected from the group of sequences consisting of PKKKRKV (SEQ ID NO: 196), KRPAATKKAGQAKKKK (SEQ ID NO: 197), PAAKRVKLD (SEQ ID NO: 248), RQRRNELKRSP (SEQ ID NO: 161), NQSSNFGPMKGGNFGGRSSGPYGGGGQYFAKPRNQGGY (SEQ ID NO: 162), RMRIZFKNKGKDTAELRRRRVEVSVELRKAKKDEQILKRRNV (SEQ ID NO: 163), VSRKRPRP (SEQ ID NO: 164), PPKKARED (SEQ ID NO: 165), PQPKKKPL (SEQ ID NO: 166), SALIKKKKKMAP (SEQ ID NO: 167), DRLRR (SEQ ID NO: 168), PKQKKRK (SEQ ID NO: 169), RKLKKKIKKL (SEQ ID NO: 170), REKKKFLKRR (SEQ ID NO: 171), KRKGDEVDGVDEVAKKKSKK (SEQ ID NO: 172), RKCLQAGMNLEARKTKK (SEQ ID NO: 173), PRPRKIPR (SEQ ID NO: 174), PPRKKRTVV (SEQ ID NO: 175), NLSKKKKRKREK (SEQ ID NO: 176), RRPSRPFRKP (SEQ ID NO: 177), KRPRSPSS (SEQ ID NO: 178), KRGINDRNFWRGENERKTR (SEQ ID NO: 179), PRPPKMARYDN (SEQ ID NO: 180), KRSFSKAF (SEQ ID NO: 181), KLKIKRPVK (SEQ ID NO: 182), PKKKRKVPPPPAAKRVKLD (SEQ ID NO: 183), PKTRRRPRRSQRKRPPT (SEQ ID NO: 184), SRRRKANPTKLSENAKKLAKEVEN (SEQ ID NO: 41827), KTRRRPRRSQRKRPPT (SEQ ID NO: 186), RRKKRRPRRKKRR (SEQ ID NO: 187), PKKKSRKPKKKSRK (SEQ ID NO: 188), HKKKHPDASVNFSEFSK (SEQ ID NO: 189), QRPGPYDRPQRPGPYDRP (SEQ ID NO: 190), LSPSLSPLLSPSLSPL (SEQ ID NO: 191), RGKGGKGLGKGGAKRHRK (SEQ ID NO: 192), PKRGRGRPKRGRGR (SEQ ID NO: 193), PKKKRKVPPPPKKKRKV (SEQ ID NO: 195), PAKRARRGYKC (SEQ ID NO: 40188), KLGPRKATGRW (SEQ ID NO: 40189), PRRKREE (SEQ ID NO: 40190), PYRGRKE (SEQ ID NO: 40191), PLRKRPRR (SEQ ID NO: 40192), PLRKRPRRGSPLRKRPRR (SEQ ID NO: 40193), PAAKRVKLDGGKRTADGSEFESPKKKRKV (SEQ ID NO: 40194), PAAKRVKLDGGKRTADGSEFESPKKKRKVGIHGVPAA (SEQ ID NO: 40195), PAAKRVKLDGGKRTADGSEFESPKKKRKVAEAAAKEAAAKEAAAKA (SEQ ID NO: 40196), PAAKRVKLDGGKRTADGSEFESPKKKRKVPG (SEQ ID NO: 40710), KRKGSPERGERKRHW (SEQ ID NO: 40198), KRTADSQHSTPPKTKRKVEFEPKKKRKV (SEQ ID NO: 41828), and PKKKRKVGGSKRTADSQHSTPPKTKRKVEFEPKKKRKV (SEQ ID NO: 40200) wherein the one or more NLS are linked to the CRISPR variant or to adjacent NLS with a linker peptide wherein the linker peptide is selected from the group consisting of RS, (G)n (SEQ ID NO: 40201), (GS)n (SEQ ID NO: 40202), (GSGGS)n (SEQ ID NO: 208), (GGSGGS)n (SEQ ID NO: 209), (GGGS)n (SEQ ID NO: 210), GGSG (SEQ ID NO: 211), GGSGG (SEQ ID NO: 212), GSGSG (SEQ ID NO: 213), GSGGG (SEQ ID NO: 214), GGGSG (SEQ ID NO: 215), GSSSG (SEQ ID NO: 216), GPGP (SEQ ID NO: 217), GGP, PPP, PPAPPA (SEQ ID NO: 218), PPPG (SEQ ID NO: 40207), PPPGPPP (SEQ ID NO: 219), PPP(GGGS)n (SEQ ID NO: 40203), (GGGS)nPPP (SEQ ID NO: 40204), AEAAAKEAAAKEAAAKA (SEQ ID NO: 40205), and TPPKTKRKVEFE (SEQ ID NO: 40206), wherein n is 1 to 5.

82. The polynucleotide of any one of claims 77-80, wherein the one or more encoded NLS are selected from the group consisting of SEQ ID NOS: 40443-40501 as set forth in Table 15 and Table 16, or a sequence having at least 85%, at least 90%, at least 95%, at least 95%, at least 96%, at least 97%, at least 98% identity thereto.

83. The polynucleotide of any one of claims 77-80, wherein the one or more encoded NLS are selected from the group of sequences consisting of SEQ ID NOS: 40443-40501 as set forth in Table 15 and Table 16.

84. The polynucleotide of any one of claims 1-83, wherein the encoded first gRNA comprises a sequence selected from the group consisting of SEQ ID NOS: 2101-2285, 39981-40026, 40913-40958, and 41817 as set forth in Table 2, or a sequence having at least 85%, at least 90%, at least 95%, at least 95%, at least 96%, at least 97%, at least 98% identity thereto.

85. The polynucleotide of any one of claims 1-84, wherein the encoded first gRNA comprises a sequence selected from the group consisting of SEQ ID NOS: 2101-2285, 39981-40026, 40913-40958, and 41817 as set forth in Table 2.

86. The polynucleotide of claim 85, wherein the encoded first gRNA comprises a targeting sequence complementary to a target nucleic acid sequence, wherein the targeting sequence has at least 15 to 30 nucleotides.

87. The polynucleotide of claim 86, wherein the targeting sequence has 18, 19, or 20 nucleotides.

88. The polynucleotide of any one of claims 1-87, comprising a sequence encoding a second gRNA and a third promoter operably linked to the second gRNA.

89. The polynucleotide of claim 88, wherein the third promoter is a pol III promoter.

90. The polynucleotide of claim 88 or claim 89, wherein the third promoter is selected from the group consisting of U6, mini U61, mini U62, mini U63, BiH1 (Bidrectional H1 promoter), BiU6 (Bidirectional U6 promoter), gorilla U6, rhesus U6, human 7sk, and human H1 promoters.

91. The polynucleotide of claim 90, wherein the third promoter is a truncated variant of the U6, mini U61, mini U62, mini U63, BiH1, BiU6, gorilla U6, rhesus U6, human 7sk, or human H1 promoters.

92. The polynucleotide of any one of claims 88-91, wherein the third promoter has less than about 250 nucleotides, less than about 220 nucleotides, less than about 200 nucleotides, less than about 160 nucleotides, less than about 140 nucleotides, less than about 130 nucleotides, less than about 120 nucleotides, less than about 100 nucleotides, less than about 80 nucleotides, or less than about 70 nucleotides.

93. The polynucleotide of any one of claims 88-91, wherein the third promoter has between about 70 to about 245 nucleotides, between about 100 to about 220 nucleotides, or between about 120 to about 160 nucleotides.

94. The polynucleotide of any one of claims 88-93, wherein the third promoter is selected from the group consisting SEQ ID NOS: 40401-40420 and 41010-41029 as set forth in Table 9, or a sequence having at least 85%, at least 90%, at least 95%, at least 95%, at least 96%, at least 97%, at least 98%, or at least 99% identity thereto.

95. The polynucleotide of any one of claims 88-94, wherein the third promoter enhances transcription of the second gRNA.

96. The polynucleotide of any one of claims 88-95, wherein the encoded second gRNA comprises a sequence selected from the group consisting of SEQ ID NOS: 2101-2285, and 39981-40026, 40913-40958, and 41817 as set forth in Table 2, or a sequence having at least 85%, at least 90%, at least 95%, at least 95%, at least 96%, at least 97%, at least 98% identity thereto.

97. The polynucleotide of any one of claims 88-95, wherein the encoded second gRNA comprises a sequence selected from the group consisting of SEQ ID NOS: 2101-2285, 39981-40026, 40913-40958, and 41817 as set forth in Table 2.

98. The polynucleotide of any one of claims 89-97, wherein the encoded second gRNA comprises a targeting sequence complementary to a target nucleic acid sequence different than the target nucleic acid of claim 86 or claim 87, wherein the targeting sequence has at least 15 to 30 nucleotides.

99. The polynucleotide of claim 98, wherein the targeting sequence has 18, 19, or 20 nucleotides.

100. The polynucleotide of any one of claims 86-99, wherein the targeting sequence is selected from the group consisting of SEQ ID NOS: 41056-41776 as set forth in Table 27, or a sequence having at least 80%, at least 90%, or at least 95% sequence identity thereto.

101. The polynucleotide of any one of claims 86-99, wherein the targeting sequence is selected from the group consisting of SEQ ID NOS: 41056-41776 as set forth in Table 27.

102. The polynucleotide of any one of claims 86-101, wherein the encoded first and second gRNA comprise a scaffold sequence having one or more modifications relative to SEQ ID NO: 2238, wherein the one or more modifications result in an improved characteristic in the expressed first and second gRNA.

103. The polynucleotide of claim 102, wherein the one or more modifications comprise one or more nucleotide substitutions, insertions, and/or deletions as set forth in Table 28.

104. The polynucleotide of claim 102 or claim 103, wherein the improved characteristic is one or more functional properties selected from the group consisting of increased editing activity, increased pseudoknot stem stability, increased triplex region stability, increased scaffold stem stability, extended stem stability, reduced off-target folding intermediates, and increased binding affinity to a Class 2, Type V CRISPR protein, optionally in an in vitro assay.

105. The polynucleotide of any one of claims 102-104, wherein the expressed gRNA scaffold exhibits an improved enrichment score (log₂) of at least about 2.0, at least about 2.5, at least about 3, or at least about 3.5 greater compared to the score of the gRNA scaffold of SEQ ID NO: 2238 in an in vitro assay.

106. The polynucleotide of claim 84-101, wherein the encoded first and second gRNA comprise a scaffold sequence having one or more modifications relative to SEQ ID NO: 2239, wherein the one or more modifications result in an improved characteristic in the expressed first and second gRNA.

107. The polynucleotide of claim 106, wherein the one or more modifications comprise one or more nucleotide substitutions, insertions, and/or deletions as set forth in Table 29.

108. The polynucleotide of claim 106 or claim 107, wherein the improved characteristic is one or more functional properties selected from the group consisting of increased editing activity, increased pseudoknot stem stability, increased triplex region stability, increased scaffold stem stability, extended stem stability, reduced off-target folding intermediates, and increased binding affinity to a Class 2, Type V CRISPR protein, optionally in an in vitro assay.

109. The polynucleotide of any one of claims 106-108, wherein the expressed gRNA scaffold exhibits an improved enrichment score (log₂) of at least about 1.2, at least about 1.5, at least about 2.0, at least about 2.5, at least about 3, or at least about 3.5 greater compared to the score of the gRNA scaffold of SEQ ID NO: 2239 in an in vitro assay.

110. The polynucleotide of any one of claims 106-109, comprising one or more modifications at positions relative to the sequence of SEQ ID NO: 2239 selected from the group consisting of C9, U11, C17, U24, A29, U54, G64, A88, and A95.

111. The polynucleotide of claim 110, comprising one or more modifications relative to the sequence of SEQ ID NO: 2239 selected from the group consisting of C9U, U11C, C17G, U24C, A29C, an insertion of G at position 54, an insertion of C at position 64, A88G, and A95G.

112. The polynucleotide of claim 111, comprising modifications relative to the sequence of SEQ ID NO: 2239 consisting of C9U, U11C, C17G, U24C, A29C, an insertion of G at position 54, an insertion of C at position 64, A88G, and A95G.

113. The polynucleotide of any one of claims 106-112, wherein the improved characteristic is selected from the group consisting of pseudoknot stem stability, triplex region stability, scaffold bubble stability, extended stem stability, and binding affinity to a Class 2, Type V CRISPR protein.

114. The polynucleotide of claim 112, wherein the insertion of C at position 64 and the A88G substitution relative to the sequence of SEQ ID NO: 2239 resolves an asymmetrical bulge element of the extended stem, enhancing the stability of the extended stem of the gRNA scaffold.

115. The polynucleotide of claim 112, wherein the substitutions of U11C, U24C, and A95G increase the stability of the triplex region of the gRNA scaffold.

116. The polynucleotide of claim 112, wherein the substitution of A29C increases the stability of the pseudoknot stem.

117. The polynucleotide of any one of claims 1-116, wherein the accessory element is a post-transcriptional regulatory element (PTRE) selected from the group consisting of cytomegalovirus immediate/early intronA, hepatitis B virus PRE (HPRE), Woodchuck Hepatitis virus PRE (WPRE), and 5′ untranslated region (UTR) of human heat shock protein 70 mRNA (Hsp70).

118. The polynucleotide of claim 117, wherein the accessory element is a PTRE selected from the group consisting SEQ ID NOS: 40431-40442 as set forth in Table 12, or a sequence having at least 85%, at least 90%, at least 95%, at least 95%, at least 96%, at least 97%, at least 98% identity thereto.

119. The polynucleotide of any one of claims 1-118, wherein the 5′ and 3′ ITRs are derived from serotype AAV1, AAV2, AAV3, AAV4, AAV5, AAV6, AAV7, AAV8, AAV9, AAV10, AAV11, AAV12, AAV 44.9, AAV-Rh74, or AAVRh10.

120. The polynucleotide of claim 119, wherein the 5′ and 3′ ITRs are derived from serotype AAV2.

121. The polynucleotide of any one of claims 1-120, comprising one or more sequences selected from the group consisting of the sequences of Tables 8-10, 12, 13, 17-22 and 24-27, or a sequence having at least 85%, at least 90%, at least 95%, at least 95%, at least 96%, at least 97%, at least 98%, or at least 99% identity thereto.

122. The polynucleotide of any one of claims 1-121, comprising one or more sequences selected from the group consisting of the sequences of Tables 8-10, 12, 13, 17-22 and 24-27.

123. The polynucleotide of any one of claims 1-122, comprising one or more sequences selected from the group consisting of the sequences of Table 26, or a sequence having at least 85%, at least 90%, at least 95%, at least 95%, at least 96%, at least 97%, at least 98%, or at least 99% identity thereto.

124. The polynucleotide of any one of claims 1-123, comprising one or more sequences selected from the group consisting of the sequences of Table 26.

125. The polynucleotide of claim 124, comprising a sequence of a construct selected from the group of constructs of 1-174, 177-186, and 188-198 as set forth in Table 26.

126. The polynucleotide of any one of claims 123-125, wherein the sequence further comprises a targeting sequence selected from the group of sequences of SEQ ID NOS: 41056-41776 as set forth in Table 27, wherein the targeting sequence is linked to the 3′ end of the polynucleotide sequence encoding the gRNA.

127. The polynucleotide of any one of claims 1-126, wherein one or more AAV component sequences selected from the group consisting of 5′ ITR, 3′ ITR, pol III promoter, pol II promoter, encoding sequence for CRISPR nuclease, encoding sequence for gRNA, accessory element, and poly(A) are modified for depletion of all or a portion of the CpG dinucleotides of the sequences

128. The polynucleotide of claim 127, wherein one or more AAV component sequences selected from the group consisting of 5′ ITR, 3′ ITR, pol III promoter, pol II promoter, encoding sequence for a CRISPR nuclease, encoding sequence for gRNA, and poly(A), and accessory element comprise less than about 10%, less than about 5%, or less than about 1% CpG dinucleotides.

129. The polynucleotide of claim 127, wherein one or more AAV component sequences selected from the group consisting of 5′ ITR, 3′ ITR, pol III promoter, pol II promoter, encoding sequence for a CRISPR nuclease, encoding sequence for gRNA, and poly(A), and accessory element are devoid of CpG dinucleotides.

130. The polynucleotide of any one of claim 127-129, wherein the one or more AAV component sequences codon-optimized for depletion of all or a portion of the CpG dinucleotides are selected from the group consisting of SEQ ID NOS: 41045-41055 as set forth in Table 25.

131. The polynucleotide of any one of claims 1-130, wherein the polynucleotide has the configuration of a construct depicted in any one of FIG. 24, 33-35, or 42.

132. A recombinant adeno-associated virus vector (rAAV) comprising:
a. an AAV capsid protein, and
b. the polynucleotide of any one of claims 1-131.

133. The rAAV of claim 132, wherein the AAV capsid protein is derived from serotype AAV1, AAV2, AAV3, AAV4, AAV5, AAV6, AAV7, AAV8, AAV9, AAV10, AAV11, AAV12, AAV 44.9, AAV-Rh74, or AAVRh10.

134. The rAAV of claim 133, wherein the AAV capsid protein and the 5′ and 3′ ITR are derived from the same serotype of AAV.

135. The rAAV of claim 133, wherein the AAV capsid protein and the 5′ and 3′ ITR are derived from different serotypes of AAV.

136. The rAAV of claim 135, wherein the 5′ and 3′ ITR are derived from AAV serotype 2.

137. The rAAV of any of claims 132-136, wherein upon transduction of a cell with the rAAV, the CRISPR protein and gRNA are capable of being expressed.

138. The rAAV of claim 137, wherein upon expression, the gRNA is capable of forming a ribonucleoprotein (RNP) complex with the CRISPR protein.

139. The rAAV of claim 137 or claim 138, wherein the AAV polynucleotide component sequences modified for depletion of all or a portion of the CpG dinucleotides substantially retain their functional properties upon expression.

140. The rAAV of claim 137 or claim 138, wherein the AAV polynucleotide component sequences modified for depletion of all or a portion of the CpG dinucleotides exhibit a lower potential for inducing an immune response compared to an rAAV wherein the AAV polynucleotide is not modified for depletion of the CpG dinucleotides.

141. The rAAV of claim 140, wherein the lower potential for inducing an immune response is exhibited in an in vitro mammalian cell assay designed to detect production of one or more markers of an inflammatory response selected from the group consisting of TLR9, interleukin-1 (IL-1), IL-6, IL-12, IL-18, tumor necrosis factor alpha (TNF-α), interferon gamma (IFNγ), and granulocyte-macrophage colony stimulating factor (GM-CSF).

142. The rAAV of claim 141, wherein the rAAV comprising the AAV polynucleotide component sequences modified for depletion of all or a portion of the CpG dinucleotides elicits reduced production of the one or more inflammatory markers of at least about 10%, at least about 20%, at least about 30%, at least about 40%, at least about 50%, at least about 60%, at least about 80%, or at least about 90% less compared to the comparable rAAV that is not CpG depleted.

143. The rAAV of claim 140, wherein administration of a dose of the rAAV comprising the AAV polynucleotide component sequences modified for depletion of all or a portion of the CpG dinucleotides to a subject elicits a reduced immune response compared to an administered dose of the comparable rAAV that is not CpG depleted.

144. The rAAV of claim 143, wherein the reduced immune response is a reduction of the production of anti-rAAV antibodies or a delayed-type hypersensitivity reaction to an rAAV component in the subject.

145. The rAAV of claim 143, wherein the reduced immune response is determined by the measurement of one or more inflammatory markers in the blood of the subject selected from the group consisting of TLR9, interleukin-1 (IL-1), IL-6, IL-12, IL-18, tumor necrosis factor alpha (TNF-α), interferon gamma (IFNγ), and granulocyte-macrophage colony stimulating factor (GM-CSF), wherein the one or more markers are reduced by at least about 10%, at least about 20%, at least about 30%, at least about 40%, at least about 50%, at least about 60%, at least about 80%, or at least about 90% compared to the comparable rAAV that is not CpG depleted.

146. The rAAV of any one of claims 143-145, wherein the subject is selected from mouse, rat, pig, dog, and non-human primate.

147. The rAAV of any one of claims 143-145, wherein the subject is human.

148. A pharmaceutical composition, comprising the rAAV of any one of claim 132 and a pharmaceutically acceptable carrier, diluent or excipient.

149. A method for modifying a target nucleic acid in a population of mammalian cells, comprising contacting a plurality of the cells with an effective amount of the rAAV of any one of claims 132-147, wherein the target nucleic acid of a gene of the cells targeted by the expressed gRNA is modified by the expressed CRISPR protein.

150. The method of claim 149, wherein the gene of the cells comprises one or more mutations.

151. The method of claim 149 or claim 150, wherein the modifying comprises introducing an insertion, deletion, substitution, duplication, or inversion of one or more nucleotides in the target nucleic acid of the cells of the population.

152. The method of any one of claims 149-151, wherein the gene is knocked down or knocked out.

153. The method of any one of claims 149-151, wherein the gene is modified such that a functional gene product can be expressed.

154. A method of treating a disease in a subject caused by one or more mutations in a gene of the subject, comprising administering a therapeutically effective dose of the rAAV of any one of claims 132-145 to the subject.

155. The method of claim 149, wherein the rAAV is administered to a subject at a dose of at least about 1×10⁸ vector genomes (vg), at least about 1×10⁵ vector genomes/kg (vg/kg), at least about 1×10⁶ vg/kg, at least about 1×10⁷ vg/kg, at least about 1×10⁸ vg/kg, at least about 1×10⁹ vg/kg, at least about 1×10¹⁰ vg/kg, at least about 1×10¹¹ vg/kg, at least about 1×10¹² vg/kg, at least about 1×10¹³ vg/kg, at least about 1×10¹⁴ vg/kg, at least about 1×10¹⁵ vg/kg, or at least about 1×10¹⁶ vg/kg.

156. The method of claim 154, wherein the rAAV is administered to a subject at a dose of at least about 1×10⁵ vg/kg to about 1×10¹⁶ vg/kg, at least about 1×10⁶ vg/kg to about 1×10¹⁵ vg/kg, or at least about 1×10⁷ vg/kg to about 1×10¹⁴ vg/kg.

157. The method of any one of claims 154-156, wherein the rAAV is administered to the subject by a route of administration selected from subcutaneous, intradermal, intraneural, intranodal, intramedullary, intramuscular, intralumbar, intrathecal, subarachnoid, intraventricular, intracapsular, intravenous, intralymphatical, intraocular or intraperitoneal routes, and wherein the administering method is injection, transfusion, or implantation.

158. The method of any one of claims 149-157, wherein the subject is selected from the group consisting of mouse, rat, pig, and non-human primate.

159. The method of any one of claims 149-157, wherein the subject is a human.

160. A method of making an rAAV vector, comprising:
a. providing a population of packaging cells; and
b. transfecting the population of cells with:
i) a vector comprising the polynucleotide of any one of claims 1-131;
ii) a vector comprising an aap (assembly) gene; and
iii) a vector comprising rep and cap genomes.

161. The method of claim 160, wherein the packaging cell is selected from the group consisting of BHK cells, HEK293 cells, HEK293T cells, NS0 cells, SP2/0 cells, YO myeloma cells, P3X63 mouse myeloma cells, PER cells, PER.C6 cells, hybridoma cells, NIH3T3 cells, COS cells, HeLa cells, and CHO cells.

162. The method of claim 160 or claim 161, the method further comprising recovering the rAAV vector.

163. The method of any one of claims 160-162, wherein the component sequences of the AAV polynucleotide are encompassed in a single rAAV particle.

164. A method of reducing the immunogenicity of an rAAV, comprising deleting all or a portion of the CpG dinucleotides of the sequences of the AAV component sequences selected from the group consisting of 5′ ITR, 3′ ITR, pol III promoter, pol II promoter, encoding sequence for CRISPR nuclease, encoding sequence for gRNA, accessory element, and poly(A).

165. The method of claim 164, wherein the one or more AAV polynucleotide component sequences comprise less than about 10%, less than about 5%, or less than about 1% CpG dinucleotides.

166. The method of claim 165, wherein one or more AAV polynucleotide component sequences are devoid of CpG dinucleotides.

167. The method of any one of claim 164-166, wherein the one or more AAV polynucleotide component sequences are selected from the group consisting of SEQ ID NOS: 41045-41055 as set forth in Table 25.

168. The method of any one of claims 164-167, wherein the rAAV exhibits a lower potential for inducing production of one or more markers of an inflammatory response in an in vitro mammalian cell assay compared to a comparable rAAV wherein the CpG dinucleotides have not been deleted, wherein the one or more inflammatory markers are selected from the group consisting of TLR9, interleukin-1 (IL-1), IL-6, IL-12, IL-18, tumor necrosis factor alpha (TNF-α), interferon gamma (IFNγ), and granulocyte-macrophage colony stimulating factor (GM-CSF).

169. The method of claim 168, wherein the rAAV elicits reduced production of the one or more inflammatory markers of at least about 10%, at least about 20%, at least about 30%, at least about 40%, at least about 50%, at least about 60%, at least about 80%, or at least about 90% less compared to the comparable rAAV that is not CpG depleted.

170. The method of any one of claims 164-167, wherein administration of a dose of the rAAV comprising the AAV polynucleotide component sequences modified for depletion of all or a portion of the CpG dinucleotides to a subject elicits a reduced immune response compared to an administered dose of the comparable rAAV that is not CpG depleted.

171. The method of claim 170, wherein the reduced immune response is a reduction of the production of anti-rAAV antibodies or a delayed-type hypersensitivity reaction to an rAAV component in the subject.

172. The method of claim 170, wherein the reduced immune response is determined by the measurement of one or more inflammatory markers in the blood of the subject selected from the group consisting of TLR9, interleukin-1 (IL-1), IL-6, IL-12, IL-18, tumor necrosis factor alpha (TNF-α), interferon gamma (IFNγ), and granulocyte-macrophage colony stimulating factor (GM-CSF), wherein the one or more markers are reduced by at least about 10%, at least about 20%, at least about 30%, at least about 40%, at least about 50%, at least about 60%, at least about 80%, or at least about 90% compared to the comparable rAAV that is not CpG depleted.

173. The method of any one of claims 164-172, wherein the subject is selected from mouse, rat, pig, dog, and non-human primate.

174. The method of any one of claims 164-172, wherein the subject is human.

175. A composition of an rAAV of any one of claims 132-147, for use as a medicament for the treatment of a human in need thereof.
